(19) United States
(12) Patent Application Publication
(10) Pub. No.: US 2006/0009456 A1
(43) Pub. Date: Jan. 12, 2006

(54) Aryl-substituted piperazine derivatives

(76) Inventors: Alan J. Hutchinson, Madison, CT (US); Bertrand L. Chenard, Waterford, CT (US); Guiying Li, Branford, CT (US); Manuka Ghosh, Madison, CT (US); James G. Tarrant, Hamden, CT (US); Taeyoung Yoon, Newton, MA (US); George P. Luke, Clinton, CT (US); Kyungae Lee, Newton, MA (US); Mary-Margaret E. O'Donnell, Shelton, CT (US); Wallace C. Pringle, Guilford, CT (US); John M. Peterson, Durham, CT (US); Kevin J. Hodgetts, Killingworth, CT (US); Cheryl K. Steenstra, Bath, ME (US); Dario Doller, Wallingford, CT (US)

Correspondence Address: Cantor Colburn LLP, 55 Griffin Road South, Bloomfield, CT, 06002, US

(21) Appl. No.: 11/154,986

(22) Filed: Jun. 16, 2005

Related U.S. Application Data
(60) Provisional application No. 60/580,958, filed on Jun. 17, 2004.

(51) Int. Cl. A61K 31/498; A61K 31/497; A61K 31/496; C07D 403/02; C07D 487/04

(52) U.S. Cl. 514/249; 514/255.05; 514/252.11; 514/253.11; 544/349; 544/405; 544/357; 544/360

(57) ABSTRACT

Aryl-substituted piperazine derivatives are provided. Such compounds may be used to modulate MCH receptor activity in vivo or in vitro, and are particularly useful in the treatment of a variety of metabolic, feeding and sexual disorders in humans, domesticated companion animals and livestock animals. Pharmaceutical compositions and methods for treating such disorders are provided, as are methods for using such ligands for detecting MCH receptors (e.g., receptor localization studies).

### CROSS-REFERENCE TO RELATED APPLICATIONS

This application claims priority from U.S. Provisional Application No. 60/580,958, filed Jun. 17, 2004, which is hereby incorporated by reference in its entirety.

### FIELD OF THE INVENTION

This invention relates generally to aryl-substituted piperazine derivatives. The invention further relates to the use of such compounds for treating a variety of metabolic, eating and sexual disorders, and as probes for the detection and localization of melanin concentrating hormone receptors.

### BACKGROUND OF THE INVENTION

Melanin concentrating hormone, or MCH, is a cyclic 19 amino acid neuropeptide first identified as a regulator of skin coloration in fish and other vertebrates, and subsequently as a regulator of food intake and energy balance in higher vertebrates. In many species, including humans, MCH is produced in the hypothalamus. MCH is also produced at various peripheral sites, including the gastrointestinal tract and testis.

The postulated role of MCH in feeding behavior and body weight regulation is confirmed by the finding that i.c.v. injection of MCH increases caloric consumption in rats over similarly treated control animals. Furthermore, rats having the ob/ob genotype exhibit a 50-80% increase in MCH mRNA expression as compared to leaner ob/+ genotype mice, and prepro-MCH knockout mice, as well as MCH receptor knockout mice, are leaner than normal mice, due to hypophagia and an increased metabolic rate.

MCH activity is mediated via binding to specific receptors. Like other G protein-coupled receptors (e.g., neuropeptide Y and beta-adrenergic receptors), MCH receptors are membrane-spanning proteins that are generally found on cell surfaces, and consist of a single contiguous amino acid chain comprising an extracellular N-terminal domain, seven membrane-spanning alpha helical domains (connected by three intracellular loop domains alternating with three extracellular loop domains), and an intracellular C-terminal domain. Signal transduction is typically initiated by the binding of extracellular MCH to the receptor, which elicits conformational changes in the extracellular domains. When the receptor is functioning properly, these conformational changes propagate through the transmembrane domains and result in a coordinated change in the intracellular portions of the receptor. This precise alteration in the intracellular domains acts to trigger the associated G-protein complex to modulate intracellular signaling.

Human Melanin Concentrating Hormone Receptor-1 (MCH1R) is a 353 amino acid, 7-transmembrane, alpha-helical, G protein-coupled receptor, initially reported as orphan receptor SLC-1. Immunohistochemistry studies of rat brain sections indicate that MCH1R is widely expressed in brain. MCH1R expression is found in olfactory tubercle, cerebral cortex, substantia nigra, basal forebrain CA1, CA2, and CA3 fields of the hippocampus, amygdala, and in nuclei of the hypothalamus, thalamus, midbrain and hindbrain. Strong signals are observed in the ventromedial and dorsomedial nuclei of the hypothalamus, two areas of the brain involved in feeding behavior. Upon binding MCH, MCH1R recombinantly expressed in HEK 293 cells mediates a dose dependent release of intracellular calcium. Cells expressing MCH1R also exhibit a pertussis toxin sensitive dose-dependent inhibition of forskolin-elevated cyclic AMP, indicating that the receptor couples to a G_{i/o} G-protein alpha subunit. Certain monkey and human MCH1R sequences, as well as various chimeric MCH1R proteins, have been disclosed in U.S. patent application Ser. No. 10/309,515 (published as 2003/0114644 on Jun. 19, 2003).

A second MCH receptor (designated MCH2R) has also been identified. MCH2R has an overall amino acid identity of more than 30% with MCH1R, and is detected specifically in the same regions of the brain as MCH1R. Monkey and canine MCH2R sequences, as well as various chimeric MCH2R proteins, have been disclosed in U.S. patent application Ser. No. 10/291,990 (which published as 2003/0148457 on Aug. 7, 2003).

Agents capable of modulating MCH receptor activity are highly desirable for the treatment of a variety of diseases and disorders, including obesity, eating disorders (e.g., bulimia and anorexia), sexual disorders (e.g., anorgasmic or psychogenic impotence) and metabolic disorders, such as diabetes. Small molecule, non-peptide antagonists of MCH receptors would be of particular value for such therapies. The present invention fulfills this need, and provides further related advantages.

### SUMMARY OF THE INVENTION

The present invention provides aryl-substituted piperazine derivatives of Formula I: 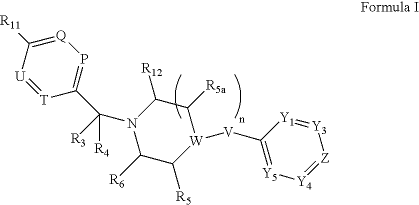
as well as pharmaceutically acceptable salts of such compounds. Within Formula I:
V is absent or —C═O—.
W is nitrogen, CH or C—OH.
Y₁, Y₃, Y₄, and Y₅ are independently optionally substituted carbon (e.g., CR₁) or nitrogen.
Z is nitrogen or optionally substituted carbon (e.g., CR₂).
Each R₁ is independently: (i) hydrogen, halogen, hydroxy, nitro, cyano, amino, aminocarbonyl, C₁-C₆alkyl, C₂-C₆alkenyl, C₂-C₆alkynyl, C₁-C₆alkoxy, haloC₁-C₆alkyl, haloC₁-C₆alkoxy, hydroxyC₁-C₆alkyl, (C₁-C₄alkoxy)C₁-C₄alkyl, C₁-C₆alkylthio, aminoC₁-C₆alkyl, mono- or di-(C₁-C₆alkyl)aminoC₀-C₆alkyl, mono- or di-(C₁-C₆alkyl)aminocarbonyl, (C₃-C₇cycloalkyl)C₀-C₆alkyl or (4- to 7-membered heterocycloalkyl)C₀-C₆alkyl; or (ii) taken together with R₂ to form a fused 5- or 6-membered carbocycle or heterocycle, each of which is optionally substituted, and preferably each of which is substituted with from 0 to 3 substituents independently chosen from halogen, hydroxy, nitro, cyano, amino, C₁-C₄alkyl, C₁-C₄alkoxy, haloC₁-C₄alkyl and haloC₁-C₄alkoxy.
R₂ is halogen, hydroxy, nitro, cyano, amino, acetyl, aminocarbonyl, imino, C₁-C₆alkyl, C₂-C₆alkenyl, C₂-C₆alkynyl, C₂-C₆alkanoyl, C₂-C₆alkyloxime, C₁-C₆alkoxy, (C₁-C₆alkoxy)C₁-C₄alkyl, hydroxyC₁-C₆alkyl, C₁-C₆alkoxycarbonyl, mono- or di-C₁-C₆alkylaminocarbonyl, C₁-C₆alkylthio, C₁-C₆alkylsulfonyl, haloC₁-C₆alkyl, haloC₁-C₆alkoxy, aminoC₁-C₆alkyl, mono- or di-(C₁-C₆alkyl)aminoC₀-C₆alkyl or (C₃-C₇cycloalkyl)C₀-C₆alkyl; or
R₂ is (4- to 7-membered heterocycloalkyl)C₀-C₆alkyl, phenylC₀-C₂alkyl, phenylC₀-C₂alkoxy or (5- or 6-membered heteroaryl)C₀-C₂alkyl, each of which is optionally substituted, and each of which is preferably substituted with from 0 to 3 substituents independently chosen from halogen, C₁-C₂alkoxy and C₁-C₂alkyl; or
R₂ is taken together with a R₁ to form a fused optionally substituted 5- or 6-membered carbocycle or heterocycle.
The variable n is 1 or 2.
R₃ is: (i) hydrogen, C₁-C₆alkyl, C₂-C₆alkenyl or haloC₁-C₆alkyl; or (ii) taken together with one or both of R₆ and R₁₀ to form a fused carbocycle or heterocycle having one or two rings, wherein each ring contains from 5 to 8 ring members and 0, 1 or 2 heteroatoms independently chosen from N, O and S, which fused carbocycle or heterocycle is optionally substituted and is preferably substituted with from 0 to 3 substituents independently chosen from halogen, oxo, C₁-C₂alkoxy and C₁-C₂alkyl.
R₄ is hydrogen, C₁-C₆alkyl or haloC₁-C₆alkyl.
R₅ is: (i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; or (ii) taken together with R₆ to form a fused, optionally substituted C₅-C₈carbocycle or 5- to 8-membered heterocycle.
Each R₅ₐ is independently: (i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; or (ii) taken together with R₆ to form a methylene or ethylene bridge.
R₆ is: (i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; (ii) taken together with R₃ to form a fused, optionally substituted heterocycle; (iii) taken together with R₅ to form a fused, optionally substituted carbocycle or heterocycle; or (iv) taken together with R₅ₐ to form a methylene or ethylene bridge.
P is N or CR₇; Q is N or CR₈; U is N or CR₉; and T is N or CR₁₀.
R₇ is: (i) hydrogen, halogen, nitro, cyano, —COOH or a group of the formula M-L-; (ii) taken together with R₈ to form a fused, optionally substituted C₅-C₆carbocycle or 5- to 6-membered heterocycle; or (iii) taken together with R)₂ to form a fused 5- or 6-membered heterocycle that is optionally substituted, and is preferably substituted with from 0 to 3 substituents independently chosen from halogen, C₁-C₂alkyl, C₁-C₂alkoxy and oxo.
R₈ is: (i) hydrogen, halogen, nitro, cyano, —COOH or a group of the formula M-L-; or (ii) taken together with R₇ to form a fused, optionally substituted C₅-C₈carbocycle or 5- to 6-membered heterocycle.
R₉ is: (i) hydrogen, halogen, nitro, cyano, —COOH or a group of the formula M-L-; or (ii) taken together with R₁₀ or R₁, to form a fused C₅-C₁₀carbocycle or a fused 5- to 10-membered heterocycle, each of which is optionally substituted and each of which is preferably substituted with from 0 to 3 substituents independently chosen from halogen, amino, nitro, cyano, hydroxy, oxo, acetyl, aminocarbonyl, imino, C₁-C₆alkyl, C₂-C₆alkenyl, C₂-C₆alkynyl, (C₃-C₇cycloalkyl)C₀-C₆alkyl, C₁-C₆alkoxy, C₁-C₆alkylthio, C₁-C₆alkylsulfonyl, (C₁-C₆alkoxy)C₁-C₄alkyl, (C₁-C₆alkoxy)C₁-C₆alkoxy, mono- and di-(C₁-C₆alkyl)aminoC₀-C₆alkyl, C₂-C₆alkanoyl, C₁-C₆alkoxycarbonyl, mono- or di-(C₁-C₆alkyl)aminocarbonyl, haloC₁-C₆alkyl, hydroxyC₁-C₆alkyl, aminoC₁-C₆alkyl and haloC₁-C₆alkoxy.
R₁₀ is: (i) hydrogen, halogen, nitro, cyano, —COOH, or a group of the formula M-L-; or (ii) taken together with R₃ or R₉ to form a fused, optionally substituted carbocycle or heterocycle.
R₁₁ is:
   (i) a group of the formula G-L-, wherein G is hydrogen, C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl, each of which is optionally substituted; in certain embodiments, G is not hydrogen, G is substituted with from 0 to 3 substituents independently chosen from halogen, amino and C₁-C₆alkyl, and G is further substituted with from 0 to 5 substituents (preferably from 1 to 5 substituents) independently chosen from Rₐ, R_{b} and R_{c}, wherein:
Rₐ is oxo, oxime, hydroxy, cyano, —COOH, —(C═O)NH₂, —NH(C═O)H, —SO₂NH₂, —(C═N)OH, or imino;
R_{b} is C₁-C₆alkoxy, (C₁-C₆alkoxy)C₁-C₆alkoxy, mono- or di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkylsulfonylamino, C₁-C₆alkoxycarbonyl, C₂-C₆alkanoylamino, arylC₁-C₆alkanoylamino, heteroarylC₁-C₆alkanoylamino, mono- or di-(C₁-C₆alkyl)aminocarbonyl or C₁-C₆alkyloxime, each of which is substituted with from 0 to 5 substituents independently chosen from halogen, amino, cyano, hydroxy, oxo, oxime, C₁-C₄alkyl, (C₁-C₄alkoxy)C₀-C₄alkyl, mono- and di-(C₁-C₄alkyl)amino, C₂-C₄alkanoyl, C₃-C₇cycloalkyl, C₁-C₄alkoxycarbonyl, haloC₁-C₂alkyl and haloC₁-C₂alkoxy; and
R_{c} is carbocycleC₀-C₆alkyl, heterocycleC₀-C₆alkyl, carbocycleC₀-C₆alkoxy, heterocycleC₀-C₆alkoxy, carbocycleC₀-C₆alkylamino or heterocycleC₀-C₆alkylamino, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy;
(ii) C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy; or
(iii) taken together with R₉ to form a fused, optionally substituted carbocycle or heterocycle. In certain embodiments, the fused carbocycle or heterocycle is substituted with at least one substituent independently chosen from halogen, amino, cyano, hydroxy, oxo, C₁-C₆alkyl, (C₁-C₆alkoxy)C₀-C₆alkyl, C₁-C₆alkoxy, (C₁-C₆alkoxy)C₁-C₆alkoxy, mono- and di-(C₁-C₆alkyl)aminoC₀-C₆alkyl, C₂-C₄alkanoyl, (C₃-C₇Cycloalkyl)C₀-C₆alkyl, C₁-C₄alkoxycarbonyl, haloC₁-C₆alkyl and haloC₁-C₆alkoxy.
R₁₂ is: (i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; or (ii) taken together with R₇ to form a fused, optionally substituted heterocycle.
Each L is independently a single covalent bond, 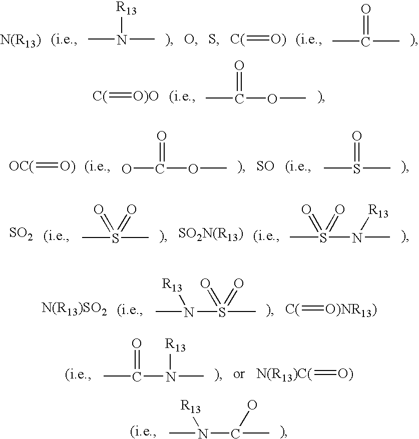
wherein each R₁₃ is independently hydrogen, C₁-C₆alkyl, C₂-C₆alkenyl, C₂-C₆alkynyl or haloC₁-C₆alkyl.
Each M is independently hydrogen, C₁-C₆alkyl, C₂-C₆alkenyl, C₂-C₆alkynyl, haloC₁-C₆alkyl, hydroxyC₁-C₆alkyl, aminoC₁-C₆alkyl, (C₁-C₆alkoxy)C₁-C₆alkyl, C₅-C₁₀cycloalkyl or 5- to 10-membered heterocycloalkyl, each of which is optionally substituted.

In certain aryl-substituted piperazine derivatives of Formula I, W is CH or C—OH. Such compounds are referred to herein as compounds of Formula I-a.

Other aryl-substituted piperazine derivatives of Formula I further satisfy Formula 1-b: 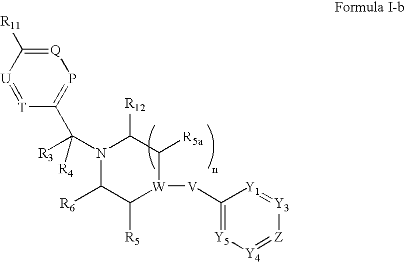
wherein:
R₅ is:
   (i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; or
   (ii) taken together with R₆ to form a fused C₅-C₈carbocycle or 5- to 8-membered heterocycle.
Each R₅ₐ is independently hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl.
R₆ is:
   (iii) taken together with R₃ to form a fused, optionally substituted heterocycle; or
   (iv) taken together with R₅ to form a fused carbocycle or heterocycle;
and the remaining variables are as described for Formula I.

Further aryl-substituted piperazine derivatives of Formula I further satisfy Formula 1-c: 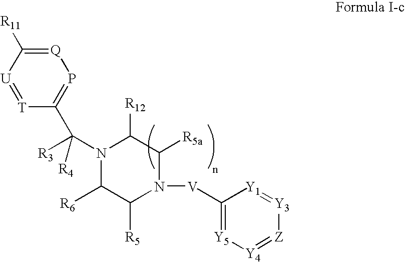
wherein
R₁₁ is:
   (i) a group of the formula G-L₁-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, and wherein G is also substituted with from 1 to 5 substituents independently chosen from Rₐ, R_{b} and R_{c};
   (ii) a group of the formula G₁-O— wherein G₁ is C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G₁ is also substituted with from 1 to 5 substituents independently chosen from Rₐ, R_{b} and R_{c};
   (iii) a group of the formula G₂-O— wherein G₂ is C₁-C₆alkyl that is substituted with from 0 to 3 amino groups, and wherein G₂ is further substituted with from 1 to 5 substituents independently chosen from Rₐ, R_{b} and R_{c}; such that R_{b} is not N-methyl, N-cyclopentylamino, and R_{c} is not (heterocycle)C₀-C₆alkyl;
   (iv) C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy; or
   (v) taken together with R₉ to form a fused optionally substituted carbocycle or heterocycle.
L₁ is independently a single covalent bond, N(R₁₃), C(═O), SO₂, SO₂NH, C(═O)N(R₁₃) or N(R₁₃)C(═O);
and the remaining variables, including Rₐ, R_{b} and R_{c}, are as described for Formula I.

Within certain aspects, aryl-substituted piperazine derivatives provided herein are MCH receptor modulators and exhibit a Kᵢ of no greater than 1 micromolar, 500 nanomolar, 100 nanomolar, or 10 nanomolar in a MCH receptor binding assay and/or have an EC₅₀ or IC₅₀ value of no greater than 1 micromolar, 500 nanomolar, 100 nanomolar, or 10 nanomolar in an assay for determining MCH receptor agonist or antagonist activity.

Within certain aspects, aryl-substituted piperazine derivatives provided herein are labeled with a detectable marker (e.g., radiolabeled or fluorescein conjugated).

The present invention further provides, within other aspects, pharmaceutical compositions comprising at least one aryl-substituted piperazine derivative provided herein in combination with a physiologically acceptable carrier or excipient. Within certain embodiments, a pharmaceutical composition provided herein may further comprise one or more additional active agents (i.e., drugs). Pharmaceutical compositions provided herein may be formulated, for example, as an injectable fluid, an aerosol, a cream, an oral liquid, a tablet, a gel, a pill, a capsule, a syrup or a transdermal patch.

Methods are further provided for modulating binding of ligand (e.g., MCH) to cellular MCH receptor, comprising contacting cells expressing MCH receptor with a MCH receptor modulator as described above, in an amount that would be sufficient to detectably modulate MCH binding to MCH receptor in vitro. The cells may, but need not, be present in a human nor non-human animal.

In other aspects, methods are provided for modulating binding of ligand (e.g., MCH) to MCH receptor in vitro, comprising MCH receptor with a MCH receptor modulator as described above, in an amount sufficient to detectably modulate MCH binding to MCH receptor.

Within further aspects, the present invention provides methods for modulating the signal-transducing activity of MCH receptor in a cell, comprising contacting a cell expressing MCH receptor, either in vivo or in vitro, with a MCH receptor modulator as described above, under conditions and in an amount that is sufficient to detectably alter the electrophysiology of the cell.

Within certain embodiments of the above methods, the MCH receptor is a MCH1R.

The present invention further provides, within other aspects, methods for treating a disease or disorder associated with MCH receptor activation, comprising administering to a patient in need of such treatment a therapeutically effective amount of a MCH receptor modulator as described above. Such diseases and disorders include, for example, obesity, eating disorders (e.g., bulimia nervosa), sexual disorders, diabetes, heart disease and stroke. The MCH receptor modulator may be administered orally, or via another means such as intranasally, intravenously or topically. Within certain embodiments, the patient is a human, companion animal (e.g., dog or cat) or livestock.

Also provided herein are methods for treating a patient, comprising diagnosing the patient as having a disease or disorder associated with MCH receptor activation, correlating the diagnosis of a disease or disorder associated with MCH receptor activation with the need for administration of a MCH receptor modulator, and administering to the patient an effective amount of a MCH receptor modulator as described above.

Methods are provided, within other aspects, for determining the presence or absence of MCH receptor in a sample, comprising: (i) contacting a sample with a compound as described above under conditions that permit binding of the compound to MCH receptor; and (ii) detecting a level of the compound bound to MCH receptor. Within certain embodiments, the compound is radiolabeled, and the step of detection comprises: (i) separating unbound compound from bound compound; and (ii) determining an amount of bound compound in the sample. Detection may be achieved, for example, using autoradiography. Representative samples include, for example, tissue sections.

Packaged pharmaceutical preparations are also provided, comprising: (a) a pharmaceutical composition as described above in a container; and (b) instructions for using the composition to treat a patient suffering from or at risk for developing a disease or disorder associated with MCH receptor activation.

In yet another aspect, methods for preparing the compounds disclosed herein, including the intermediates, are also provided herein.

These and other aspects of the present invention will become apparent upon reference to the following detailed description.

### DETAILED DESCRIPTION OF THE INVENTION

As noted above, the present invention provides aryl-substituted piperazine derivatives of Formula I. Certain preferred compounds are MCH receptor modulators that may be used in vitro or in vivo, to inhibit MCH binding to MCH receptors, activate MCH receptors, or to otherwise modulate MCH receptor activity in a variety of contexts, as discussed in further detail below.

Terminology

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Compound descriptions are intended to encompass compounds with all possible isotopes of atoms occurring in the compounds. Isotopes are those atoms having the same atomic number but different mass numbers. By way of general example, and without limitation, isotopes of hydrogen include tritium and deuterium and isotopes of carbon include ¹¹C, ¹³C and ¹⁴C. Certain compounds are described herein using a general formula that includes variables (e.g., X, V, R₃). Unless otherwise specified, each variable within such a formula is defined independently of any other variable, and any variable that occurs more than one time in a formula is defined independently at each occurrence. In general, the variables may have any definition described herein that results in a stable compound.

The term “aryl-substituted piperazine derivative” refers to any compound that satisfies Formula I, or is a pharmaceutically acceptable salt of such a compound. Certain aryl-substituted piperazine derivatives further satisfy one or more additional formulas provided herein; the phrase “aryl-substituted piperazine derivative of Formula X” is intended to encompass both compounds of Formula X and the pharmaceutically acceptable salts of such compounds.

A “pharmaceutically acceptable salt” of a compound recited herein is an acid or base salt that is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂), —COOH where n is 0-4, and the like. Similarly, pharmaceutically acceptable cations include, but are not limited to sodium, potassium, calcium, aluminum, lithium, and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

It will be apparent that each aryl-substituted piperazine derivative may, but need not, be formulated as a hydrate, solvate or non-covalent complex. In addition, the various crystal forms and polymorphs are within the scope of the present invention. Also provided herein are prodrugs of the aryl-substituted piperazine derivatives provided herein. A “prodrug” is a compound that may not fully satisfy the structural requirements of the compounds provided herein, but is modified in vivo, following administration to a patient, to produce an aryl-substituted piperazine derivative. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxyl, amino or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

“Acetyl” refers to a group of the formula —(C═O)CH₃.

As used herein, the term “alkyl” refers to a straight or branched chain saturated aliphatic hydrocarbon. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl and 3-methylpentyl. “C₀-Cₙalkyl” refers to a single covalent bond (C₀) or an alkyl group having from 1 to n carbon atoms; for example, “C₀-C₆alkyl” refers to a single covalent bond or a C₁-C₆alkyl group. In some instances, a substituent of an alkyl group is specifically indicated. For example, “hydroxyC₁-C₆alkyl” refers to a C₁-C₆alkyl group that has at least one hydroxy substituent; aminoC₁-C₆alkyl refers to a C₁-C₆alkyl group that has at least one amino substituent.

“Alkylene” refers to a divalent alkyl group, as defined above. C₀-C₄alkylene is a single covalent bond or an alkylene group having from 1 to 4 carbon atoms.

“Alkenyl” refers to straight or branched chain alkene groups, which comprise at least one unsaturated carbon-carbon double bond. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

A “cycloalkyl” is a group that comprises one or more saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Certain cycloalkyl groups are C₃-C₇cycloalkyl, in which the ring contains from 3 to 7 ring members. Cycloalkyl groups that comprise at least one carbon-carbon double bond are specifically designated “cycloalkenyl” (e.g., 5- to 10-membered cycloalkenyl). A “cycloalkylC₀-Cₙalkyl” is a cycloalkyl group linked via a single covalent bond or a C₁-Cₙalkylene group (e.g., C₃-C₇cycloalkyl)C₀-C₆alkyl). “C₅-C₁₀cycloalkenyl” indicates a partially saturated cycloalkyl group having from 5 to 10 ring members.

By “alkoxy,” as used herein, is meant an alkyl group as described above attached via an oxygen bridge. Alkoxy groups include C₁-C₆alkoxy and C₁-C₄alkoxy groups, which have from 1 to 6 or from 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups. Similarly, “alkylthio” refers to an alkyl group as described above attached via a sulfur bridge.

“Alkylsulfonyl” refers to groups of the formula —(SO₂)-alkyl, in which the sulfur atom is the point of attachment. Alkylsulfonyl groups include C₁-C₆alkylsulfonyl and C₁-C₄alkylsulfonyl groups, which have from 1 to 6 or from 1 to 4 carbon atoms, respectively. Methylsulfonyl is one representative alkylsulfonyl group.

The term “oxo,” as used herein, refers to a keto group (C═O). An oxo group that is a substituent of a nonaromatic carbon atom results in a conversion of —CH₂— to —C(═O)—. An oxo group that is a substituent of an aromatic carbon atom results in a conversion of —CH— to —C(═O)— and a loss of aromaticity.

Similarly, “oxime” refers to a group of the formula C═NOH. An oxime group that is a substituent of a nonaromatic carbon atom results in a conversion of —CH₂— to —C(═NOH)—. “Alkyloxime” is an alkyl group as described above attached via a —C═NOH)— linker.

The term “alkanoyl” refers to an acyl group (e.g., —C═O)-alkyl). Alkanoyl groups have the indicated number of carbon atoms, with the carbon of the keto group being included in the numbered carbon atoms. For example, a C₂alkanoyl group is an acetyl group having the formula —(C═O)CH₃. Alkanoyl groups include, for example, C₂-C₈alkanoyl, C₂-C₆alkanoyl and C₂-C₄alkanoyl groups, which have from 2 to 8, from 2 to 6 or from 2 to 4 carbon atoms, respectively. “C₁alkanoyl” refers to —C═O)H, which (along with C₂-C₈alkanoyl) is encompassed by the term “C₁-C₈alkanoyl.”

“(Alkoxy)alkyl” refers to a linear or branched ether substituent (i.e., an alkyl group that is substituted with an alkoxy group). Such groups include (C₁-C₄alkoxy)C₁-C₆alkyl and (C₁-C₄alkoxy)C₁-C₄alkyl. A (C₁alkoxy)C₁alkyl group has the structure —CH₂—O—CH₃.

The term “alkoxycarbonyl” refers to an alkoxy group attached through a keto (—(C═O)—) bridge (i.e., a group having the general structure —C(═O)—O-alkyl). Alkoxycarbonyl groups include C₁-C₈, C₁-C₆ and C₁-C₄alkoxycarbonyl groups, which have from 1 to 8, 6 or 4 carbon atoms, respectively, in the alkyl portion of the group (i.e., the carbon of the keto bridge is not included in the indicated number of carbon atoms). “C₁alkoxycarbonyl” refers to —C(═O)—O—CH₃; C₃alkoxycarbonyl indicates —C(═O)—O—(CH₂)₂CH₃ or —C(═O)—O—(CH)(CH₃)₂.

“Alkanoylamino,” as used herein, refers to an alkanoyl group attached through an amino linker (i.e., a group having the general structure —N(R)C(═O)-alkyl), in which R is hydrogen or C₁-C₆alkyl. Alkanoylamino groups include C₂-C₈, C₂-C₆ and C₂-C₄alkanoylamino groups, which have from 2 to 8, 6 or 4 carbon atoms, respectively.

“Alkylamino” refers to a secondary or tertiary amine having the general structure —NH-alkyl or —N(alkyl)(alkyl), wherein each “alkyl” is selected independently from alkyl, cycloalkyl and (cycloalkyl)alkyl groups. Such groups include, for example, mono- and di-(C₁-C₈alkyl)amino groups, as well as mono- and di-(C₁-C₆alkyl)amino groups and mono- and di-(C₁-C₄alkyl)amino groups.

“Alkylaminoalkyl” refers to an alkylamino group linked via an alkylene group (i.e., a group having the general structure -alkylene-NH-alkyl or -alkylene-N(alkyl)(alkyl)) in which each alkyl is selected independently from alkyl, cycloalkyl and (cycloalkyl)alkyl groups. Alkylaminoalkyl groups include, for example, mono- and di-(C₁-C₈alkyl)aminoC₁-C₈alkyl, mono- and di-(C₁-C₆alkyl)aminoC₁-C₆alkyl and mono- and di-(C₁-C₆alkyl)aminoC₁-C₄alkyl. “Mono- or di-(C₁-C₆alkyl)aminoC₀-C₆alkyl” refers to a mono- or di-(C₁-C₆alkyl)amino group linked via a single covalent bond or a C₁-C₆alkylene group. The following are representative alkylaminoalkyl groups:

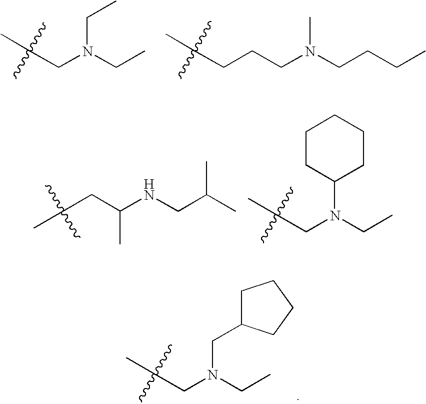

It will be apparent that the definition of “alkyl” as used in the terms “alkylamino” and “alkylaminoalkyl” differs from the definition of “alkyl” used for all other alkyl-containing groups, in the inclusion of cycloalkyl and (cycloalkyl)alkyl groups (e.g., (C₃-C₇cycloalkyl)C₀-C₆alkyl).

The term “aminocarbonyl” refers to an amide group (i.e., —(C═O)NH₂). “Mono- or di-(C₁-C₈alkyl)aminocarbonyl” is an aminocarbonyl group in which one or both of the hydrogen atoms is replaced with C₁-C₈alkyl. If both hydrogen atoms are so replaced, the alkyl groups may be the same or different.

“Aminosulfonyl” refers to groups of the formula —(SO₂)—NH₂, in which the sulfur atom is the point of attachment. The term “mono- or di-(C₁-Cₙalkyl)aminosulfonyl” refers to groups that satisfy the formula —(SO₂)—NR₂, in which the sulfur atom is the point of attachment, and in which one R is C₁-Cₙalkyl and the other R is hydrogen or an independently chosen C₁-Cₙalkyl.

The term “halogen” refers to fluorine, chlorine, bromine or iodine.

A “haloalkyl” is an alkyl group that is substituted with 1 or more independently chosen halogens (e.g., “C₁-C₈haloalkyl” groups have from 1 to 8 carbon atoms; “C₁-C₆haloalkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl. The term “haloalkoxy” refers to a haloalkyl group as defined above attached via an oxygen bridge. “C₁-C₆haloalkoxy” groups have 1 to 6 carbon atoms.

A dash (“-”) that is not between two letters or symbols is used to indicate a point of attachment for a substituent. For example, —CONH₂ is attached through the carbon atom.

A “carbocycle” or “carbocyclic group” comprises at least one ring formed entirely by carbon-carbon bonds (referred to herein as a carbocyclic ring), and does not contain a heterocycle. Unless otherwise specified, each ring within a carbocycle may be independently saturated, partially saturated or aromatic, and is optionally substituted as indicated. A carbocycle generally has from 1 to 3 fused, pendant or spiro rings; carbocycles within certain embodiments have one ring or two fused rings. Typically, each ring contains from 3 to 8 ring members (i.e., C₃-C₈); C₅-C₇ rings are recited in certain embodiments. Carbocycles comprising fused, pendant or spiro rings typically contain from 9 to 14 ring members. Certain carbocycles are C₄-C₁₀ (i.e., contain from 4 to 10 ring members and 1 or two rings). Certain representative carbocycles are cycloalkyl as described above. Other carbocycles are aryl (i.e., contain at least one aromatic carbocyclic ring, with or without one or more additional aromatic and/or cycloalkyl rings). Such aryl carbocycles include, for example, phenyl, naphthyl (e.g., 1-naphthyl and 2-naphthyl), biphenyl, fluorenyl, indanyl and 1,2,3,4-tetrahydro-naphthyl. In certain embodiments, preferred carbocycles are carbocycles having a single ring, such as phenyl and 3- to 7-membered cycloalkyl groups.

Certain carbocycles are attached via an indicated linker group (e.g., (carbocycle)alkyl, (carbocycle)alkoxy and (carbocycle)alkylamino groups). In each case the carbocycle is a substituent of the indicated linker group, each of which carries the definition set forth above. “CarbocycleC₀-C₆alkylamino” refers to a carbocycle linked via an amino (—NH—) linker or via a mono- or di-(C₁-C₆alkyl)amino group in which the point of attachment of the carbocycle may be at any carbon atom in a mono- or di-(C₁-C₆alkyl)amino group or at the nitrogen atom in a mono-(C₁-C₆alkyl)amino group.

As used herein, the term “aryl” indicates aromatic groups containing only carbon in the aromatic ring or rings. Such aromatic groups may be further substituted with carbon and/or non-carbon atoms or groups. Typical aryl groups contain 1 or 2 separate, fused, or pendant rings and from 6 to about 12 ring atoms, without heteroatoms as ring members. Aryl groups include those in which an aromatic ring is fused to a 5 to 7-membered saturated or partially saturated cyclic group that optionally contains 1 or 2 heteroatoms independently chosen from N, O and S (e.g., a 3,4-methylenedioxy-phenyl group.

The term “arylalkyl” refers to an aryl group linked via an alkylene bridge. For example, phenylC₀-C₂alkyl indicates a phenyl group that is attached via a single covalent bond (phenylC₀alkyl) or attached through an alkylene group having 1 or 2 carbon atoms. Similarly, an aryl group may be attached through other linker groups; such groups include, for example, arylC₁-C₆alkanoylamino and arylalkoxy groups, in which the aryl is attached via the indicated linker group.

A “heterocycle” or “heterocyclic group” has from 1 to 3 fused, pendant or spiro rings, at least one of which is a heterocyclic ring (i.e., one or more ring atoms is a heteroatom independently chosen from O, S and N, with the remaining ring atoms being carbon). Additional rings, if present, may be heterocyclic or carbocyclic. Typically, a heterocyclic ring comprises 1, 2, 3 or 4 heteroatoms; within certain embodiments each heterocyclic ring has 1 or 2 heteroatoms per ring. Each heterocyclic ring generally contains from 3 to 8 ring members (rings having from 4 or 5 to 7 ring members are recited in certain embodiments) and heterocycles comprising fused, pendant or spiro rings typically contain from 9 to 14 ring members. Certain heterocycles comprise a sulfur atom as a ring member; in certain embodiments, the sulfur atom, is oxidized to SO or SO₂. Heterocycles may be optionally substituted with a variety of substituents, as indicated. Unless otherwise specified, a heterocycle may be a heterocycloalkyl group (i.e., each ring is saturated or partially saturated) or a heteroaryl group (i.e., at least one heterocyclic ring within the group is aromatic), such as a 5- to 10-membered heteroaryl (which may be monocyclic or bicyclic) or a 6-membered heteroaryl (e.g., pyridyl or pyrimidyl). N-linked heterocyclic groups are linked via a component nitrogen atom. 4- to 7-membered heterocycloalkyl groups include, for example, piperidinyl, piperazinyl, pyrrolidinyl, azepanyl, morpholino, thiomorpholino and 1,1-dioxo-thiomorpholin-4-yl. Representative aromatic heterocycles include azocinyl, pyridyl, pyrimidyl, imidazolyl and tetrazolyl. In certain embodiments, preferred heterocycles are 5- to 7-membered heterocycles having a single saturated, partially unsaturated or aromatic heterocyclic ring with 5 to 7 ring members, 1 or 2 ring members independently chosen from N, O and S, with remaining ring members being carbon.

Certain heterocycles are attached via an indicated linker group (e.g., (heterocycle)alkyl, (heterocycle)alkoxy and (heterocycle)alkylamino groups). In each case the heterocycle is covalently bound to the indicated linker group, each of which carries the definition set forth above.

As used herein, “heteroaryl” indicates a monocyclic, bicyclic or tricyclic ring system that comprises at least one 5- or 6-membered heterocyclic aromatic ring that contains from 1 to 4 (preferably from 1 to 3) heteroatoms independently chosen from N, O and S, with remaining ring atoms being carbon. If the total number of S and O atoms in the heteroaryl group exceeds 1, these heteroatoms are not adjacent to one another. It is generally preferred that the total number of S and O atoms in the heteroaryl group is not more than 2; in certain embodiments, the total number of S and O atoms in the aromatic heterocycle is not more than 1. Examples of heteroaryl groups include, but are not limited to, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

A “heterocyclolalkyl” group is a heterocycle as described above, which is fully or partially saturated. In certain embodiments preferred heterocycloalkyl groups are 5- to 7-membered heterocycloalkyl groups having a single saturated ring with 5 to 7 ring members, 1 or 2 ring members independently chosen from N, O and S, and remaining ring members being carbon. A “heterocycloalkylC₀-Cₙalkyl” is a heterocycloalkyl group linked via a single covalent bond or C₁-Cₙalkylene group, such as a C₁-C₄alkylene group. A “5- to 10-membered heterocycloalkenyl” is a partially saturated heterocycloalkyl group having from 5 to 10 ring members.

A “substituent,” as used herein, refers to a molecular moiety that is covalently bonded to an atom within a molecule of interest. For example, a ring substituent may be a moiety such as a halogen, alkyl group, haloalkyl group or other group discussed herein that is covalently bonded to an atom (preferably a carbon or nitrogen atom) that is a ring member. Substituents of aromatic groups are generally covalently bonded to a ring carbon atom. The term “substitution” refers to replacing a hydrogen atom in a molecular structure with a substituent, such that the valence on the designated atom is not exceeded, and such that a chemically stable compound (i.e., a compound that can be isolated, characterized, and tested for biological activity) results from the substitution.

Groups that are “optionally substituted” are unsubstituted or are substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “MCH receptor” refers to any naturally-occurring mammalian (especially human, monkey, or canine) MCH type 1 or type 2 receptor, as well as chimeric receptors in which one or more domains of a naturally-occurring MCH1R or MCH2R are replaced with a corresponding domain of a different G protein-coupled receptor, such that the ability of the chimeric receptor to bind MCH and mediate a dose-dependent release of intracellular calcium is not diminished. MCH receptors for use within the various assays and other methods described herein include, for example, recombinantly expressed human MCH receptor (e.g., Genbank Accession No. Z86090; SEQ ID NO:29 of U.S. Patent Application Publication No. 2003/0148457), monkey MCH receptor (e.g., SEQ ID NO:2, 34 or 36 of U.S. Patent Application Publication No. 2003/0114644) or canine MCH receptor (e.g., SEQ ID NO:39 of U.S. Patent Application Publication No. 2003/0114644). Chimeric MCH receptors that may be used as described herein include, for example, those disclosed in U.S. Patent Application Publication Nos. 2003/0114644 and 2003/0148457.

A “MCH receptor modulator,” also referred to herein as a “modulator,” is a compound that alters (increases or decreases) MCH receptor activation and/or MCH receptor-mediated signal transduction. MCH receptor modulators specifically provided herein are aryl-substituted piperazine derivatives. A modulator may be a MCH receptor agonist or antagonist. In certain embodiments, a modulator may exhibit an EC₅₀ or IC₅₀ at MCH receptor that is less than 1 micromolar, 500 nM, 200 nM, 100 nM, 50 nM, 25 nM or 10 nM in a standard calcium mobilization assay (as described in Example 37, herein) and/or an agonist-stimulated GTP gamma³⁵S binding assay (as described in Example 35, herein). A modulator may be a MCH receptor agonist or antagonist, although, for certain purposes described herein, a modulator preferably inhibits MCH receptor activation resulting from binding of MCH (i.e., the modulator is an antagonist).

A MCH receptor modulator binds with “high affinity” if the Kᵢ at a MCH receptor is less than 1 micromolar, preferably less than 500 nanomolar, 100 nanomolar or 10 nanomolar. A modulator binds “specifically” to MCH receptor if it binds to a MCH receptor (total binding minus nonspecific binding) with a Kᵢ that is 10-fold, preferably 100-fold, and more preferably 1000-fold, less than the Kᵢ measured for modulator binding to other G protein-coupled receptors. For example, a modulator may have a Kᵢ of 500 nanomolar or less in an MCH receptor ligand binding assay and a Kᵢ of at least 1 micromolar in a dopamine receptor ligand binding assay, such as the assay described in Example 7 (pages 111-112) of PCT International Publication Number WO 02/094799, which is hereby incorporated by reference. Representative assays for determining Kᵢ at MCH receptor are provided in Examples 33 and 36, herein.

A modulator is considered an “antagonist” if it detectably inhibits MCH binding to MCH receptor and/or MCH-mediated signal transduction (using, for example, the representative assay provided in Example 33 or Example 36); in general, such an antagonist has a IC₅₀ value of less than 1 micromolar, preferably less than 100 nanomolar, and more preferably less than 10 nanomolar within the assay provided in Example 33 and/or the assay provided in Example 36. MCH receptor antagonists include neutral antagonists and inverse agonists.

An “inverse agonist” is a compound that reduces the activity of MCH receptor below its basal activity level in the absence of added ligand. Inverse agonists may also inhibit the activity of MCH at MCH receptor, and/or may also inhibit binding of MCH to MCH receptor. The ability of a compound to inhibit the binding of MCH to MCH receptor may be measured by a binding assay, such as the binding assays given in Examples 33 or 36. The basal activity of MCH receptor, as well as the reduction in MCH receptor activity due to the presence of antagonist, may be determined from a calcium mobilization assay, such as the assay of Example 37, or an agonist-stimulated GTP gamma³⁵S binding assay, such as the assay described in Example 35.

A “neutral antagonist” of MCH receptor is a compound that inhibits the activity of MCH at MCH receptor, but does not significantly change the basal activity of the receptor (e.g., within an assay as described in Example 35 or Example 37 performed in the absence of ligand, MCH receptor activity is reduced by no more than 10%, more preferably by no more than 5%, and even more preferably by no more than 2%; most preferably, there is no detectable reduction in activity). Neutral antagonists may also inhibit ligand binding to MCH receptor.

As used herein a “MCH receptor agonist” is a compound that elevates the activity of the receptor above the basal activity level of the receptor (i.e., enhances MCH receptor activation and/or MCH receptor-mediated signal transduction). MCH receptor agonist activity may be identified using the representative assays provided in Examples 35 and 37. In general, such an agonist has an EC₅₀ value of less than 1 micromolar, preferably less than 100 nanomolar, and more preferably less than 10 nanomolar within one or both of the assays provided in Examples 35 and 37.

A “therapeutically effective amount” (or dose) is an amount that, upon administration, is sufficient to provide a discernible patient benefit. For example, a therapeutically effective amount may reduce symptom severity or frequency, and/or may result in detectable weight loss. Alternatively, or in addition, a therapeutically effective amount may improve patient status or outcome and/or prevent or delay disease or symptom onset. A therapeutically effective amount or dose generally results in a concentration of compound in a body fluid (such as blood, plasma, serum, CSF, synovial fluid, lymph, cellular interstitial fluid, tears or urine) that is sufficient to alter the binding of ligand to MCH receptor in vitro (using an assay provided in Example 33 or Example 36) and/or MCH-mediated signal transduction (using an assay provided in Example 35 or Example 37).

A “disease or disorder associated with MCH receptor activation,” as used herein is any condition that is characterized by inappropriate stimulation of MCH receptor, regardless of the amount of MCH present locally, and/or that is responsive to modulation of MCH receptor activity (i.e., the condition or a symptom thereof is alleviated by such modulation). Such conditions include, for example, metabolic disorders (such as diabetes), heart disease, stroke, eating disorders (such as obesity and bulimia nervosa) and sexual disorders such as anorgasmic and psychogenic impotence, as well as other diseases and disorders recited herein.

A “patient” is any individual treated with an aryl-substituted piperazine derivative as provided herein. Patients include humans, as well as other animals such as companion animals (e.g., dogs and cats) and livestock. Patients may be experiencing one or more symptoms of a condition responsive to MCH receptor modulation, or may be free of such symptom(s) (i.e., treatment may be prophylactic).

Aryl-Substituted Piperazine Derivatives

As noted above, the present invention provides aryl-substituted piperazine derivatives of Formula I. Certain such compounds are MCH receptor modulators, which may be specific for a particular MCH receptor (e.g., type 1 or type 2) or may inhibit or enhance ligand binding to multiple MCH receptors. MCH receptor modulators may be used to modulate MCH receptor activity in vivo, especially in the treatment of metabolic, feeding and sexual disorders in humans, domesticated companion animals and livestock animals. Modulators may also be used within a variety of in vitro assays, such as assays for receptor activity, as probes for detection and localization of MCH receptors and as standards in assays of MCH binding and MCH-mediated signal transduction. The MCH receptor modulators provided herein are generally multi-aryl (i.e., have a plurality of unfused or fused aryl groups), non-peptide and amino acid free, and detectably modulate MCH receptor activity at submicromolar concentrations, preferably at subnanomolar concentrations.

Certain aryl-substituted piperazine derivatives further satisfy Formula I-a, I-b or I-c, as described above. Other aryl-substituted piperazine derivatives further satisfy one or more of Formulas II-VII: 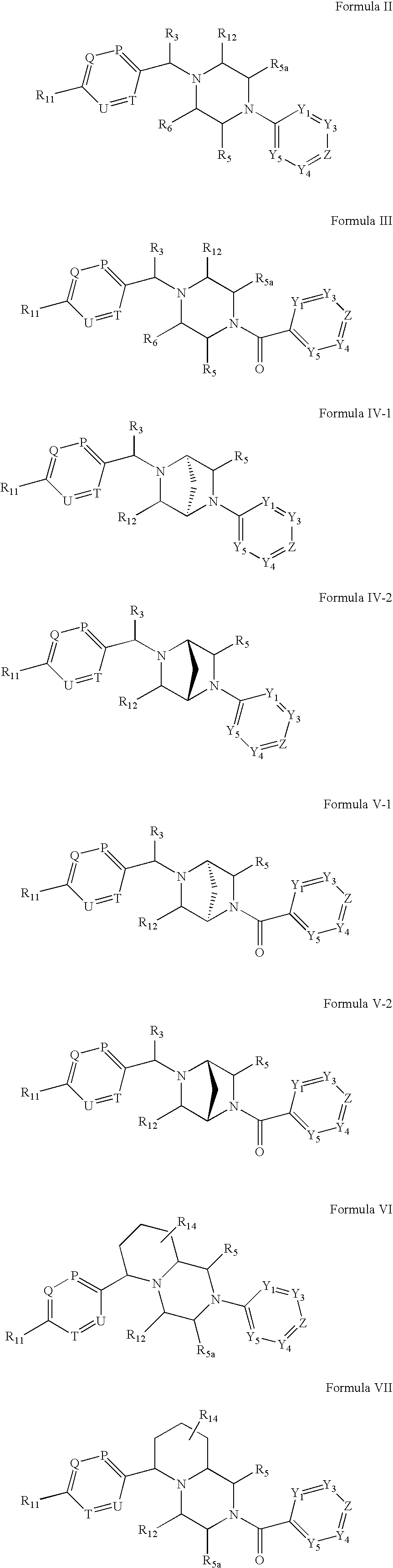
Within Formulas II-VII:
R₃ (of Formulas II-V) is hydrogen, C₁-C₂alkyl or haloC₁-C₂alkyl;
Each R₅, R₅ₐ and R₆ of Formulas II and III is independently hydrogen, C₁-C₂alkyl or C₁-C₂alkoxy;
R₁₂ is hydrogen, C₁-C₂alkyl or C₁-C₂alkoxy;
R₁₄ (in Formulas VI and VII) represents from 0 to 3 substituents independently chosen from halogen, C₁-C₂alkyl, C₁-C₂alkoxy and oxo; in certain embodiments R₁₄ is absent; and the remaining variables are as defined above.

Further provided herein are aryl-substituted piperazine derivatives of Formula I-VII, wherein the variables satisfy one or more of the following conditions:
W is nitrogen.
W is CH.
V is absent.
V is —(C═O)—.
The variable n is 1.
R₅ is: (a) hydrogen, C₁-C₂alkyl or C₁-C₂alkoxy; or (b) taken together with R₆ to form a methylene or ethylene bridge.
R₆ is (a) hydrogen, C₁-C₂alkyl or C₁-C₂alkoxy; or (b) taken together with R₃ to form a fused heterocycloalkyl; or (c) taken together with R₅ to form a methylene or ethylene bridge.
R₁₂ is (a) hydrogen, halogen, C₁-C₂alkyl or C₁-C₂alkoxy; or (b) hydrogen, C₁-C₂alkyl or C₁-C₂alkoxy.
R₁ is hydrogen and R₂ is trifluoromethyl.
Y₃ is carbon substituted with methoxy and R₂ is halogen.
Y₃ is carbon substituted with methoxy; Y₁, Y₄ and Y₅ are each CH; and R₂ is halogen.
Y₃ is CR₁, wherein the R₁ of Y₃ is taken together with R₂ to form a 6-membered aryl ring that is substituted with from 0 to 3 substituents independently chosen from halogen, hydroxy, nitro, cyano, amino, C₁-C₄alkyl, C₁-C₄alkoxy, haloC₁-C₄alkyl and haloC₁-C₄alkoxy.
Y₃ is N, and Y₁, Y₄ and Y₅ are each CH.
Y₃ and Y₄ are N, and Y, and Y₅ are each CH.
R₄ is hydrogen or methyl.
R₃ is methyl and R₄ is hydrogen.
R₅, R₆ (when present) and R₁₂ are independently hydrogen or methyl.
R₅, & and R₁₂ are hydrogen.
Z is CR₂.
Y₁ Y₃, Y₄ and Y₅ are CR₁, and Z is CR₂ (i.e., Formula VIII): 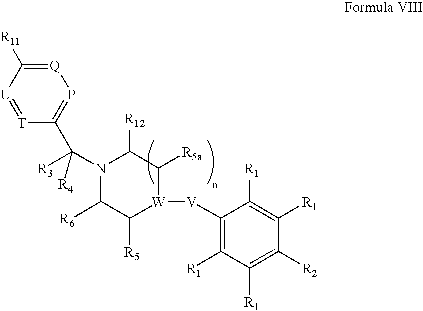
Y₁, Y₄ and Y₅ are CH, Y₃ is CR₁, and Z is CR₂ (i.e., Formula IX): 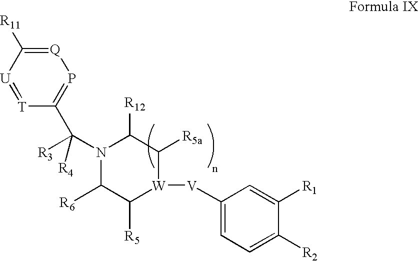
Y₁ is nitrogen, Y₃, Y₄, and Y₅ are CR₁, and Z is CR₂, (i.e., Formula X): 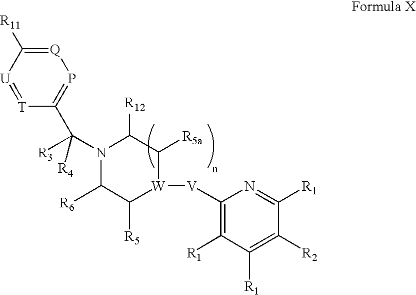
Y₁ and Y₄ are nitrogen, Y₃ and Y₅ are CR₁, and Z is CR₂ (i.e., Formula XI): 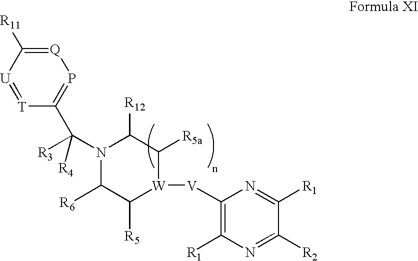
Y₄ is nitrogen, Y₁, Y₃ and Y₅ are CR₁, and Z is CR₂ (e.g., Formula XII): 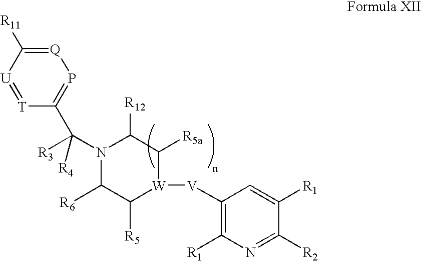
   The R₁ Variable

Within certain aryl-substituted piperazine derivatives of Formula I, and the subformulas thereof, each R₁ is independently hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, hydroxyC₁-C₆alkyl, C₁-C₆alkylthio, C₁-C₈alkylether, aminoC₁-C₆alkyl, mono- or di-(C₁-C₆alkyl)aminoC₀-C₆alkyl, mono- or di-C₁-C₆alkylaminocarbonyl, (C₃-C₇cycloalkyl)C₀-C₆alkyl or (4- to 7-membered heterocycloalkyl)C₀-C₆alkyl. Within further aryl-substituted piperazine derivatives, each R₁ is independently hydrogen, halogen, hydroxy, cyano, C₁-C₄alkyl, C₂-C₄alkenyl, C₁-C₄alkoxy, haloC₁-C₂alkyl, haloC₁-C₂alkoxy, or mono- or di-(C₁-C₂alkyl)amino. Additionally, aryl-substituted piperazine derivatives are provided wherein each R₁ is independently hydrogen, halogen, C₁-C₂alkyl, C₁-C₂alkoxy or trifluoromethyl.

The R₂ Variable

Within certain aryl-substituted piperazine derivatives of Formula I, and the subformulas thereof, R₂ is halogen, nitro, cyano, amino, acetyl, aminocarbonyl, imino, C₁-C₆alkyl, C₂-C₆alkenyl, C₂-C₆alkynyl, C₂-C₆alkanoyl, C₂-C₆alkyloxime, C₁-C₆alkoxy, (C₁-C₆alkoxy)C₁-C₄alkyl, hydroxyC₁-C₆alkyl, C₁-C₆alkoxycarbonyl, mono- or di-C₁-C₆alkylaminocarbonyl, C₁-C₆alkylthio, C₁-C₆alkylsulfonyl, haloC₁-C₆alkyl, haloC₁-C₆alkoxy, aminoC₁-C₆alkyl, mono- or di-(C₁-C₆alkyl)aminoC₀-C₆alkyl or (C₃-C₇cycloalkyl)C₀-C₆alkyl. Within further such aryl-substituted piperazine derivatives, R₂ is hydrogen, halogen, hydroxy, cyano, C₁-C₄alkyl, C₂-C₄alkenyl, C₁-C₄alkoxy, C₁-C₂alkylthio, haloC₁-C₂alkyl, haloC₁-C₂alkoxy, or mono- or di-(C₁-C₂alkyl)amino. Within still further such aryl-substituted piperazine derivatives, R₂ is halogen, C₁-C₄alkyl, C₁-C₄alkoxy or trifluoromethyl. For example, R₂ is trifluoromethyl in certain compounds, including those in which each R₁ is hydrogen. In other compounds, R₂ is a halogen and Y₄ is CR₁; in certain such compounds, the R₁ at the Y₄ position is methoxy.

The Variables P, Q, U and T

Within certain aryl-substituted piperazine derivatives of Formula I (and the subformulas thereof), the variables P, Q, U and T satisfy one of the following conditions:
P is CR₇, Q is CR₈, U is CR₉, and T is nitrogen (i.e., Formula XIII): 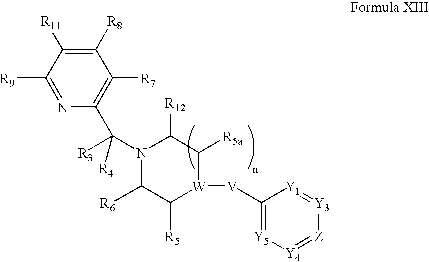
P is CR₇, Q is CR₈, U is nitrogen, and T is CR₁₀ (i.e., Formula XIV): 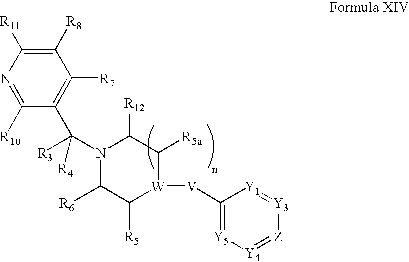
P is CR₇, Q is nitrogen, U is nitrogen, and T is CR₁₀ (i.e., Formula XV): 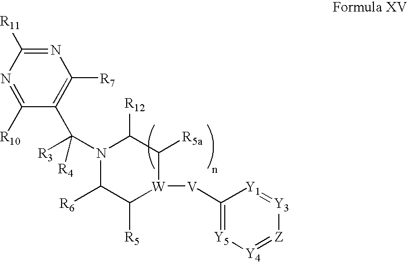
P is nitrogen, Q is CR₈, U is nitrogen, and T is CR₁₀ (i.e., Formula XVI): 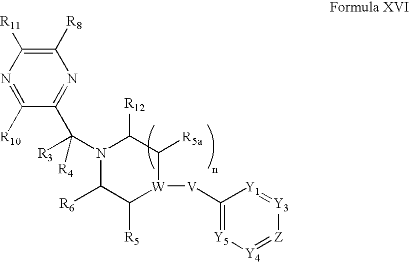
P is CR₇, Q is CR₈, U is CR₉, and T is CR₁₀ (i.e., Formula XVII):

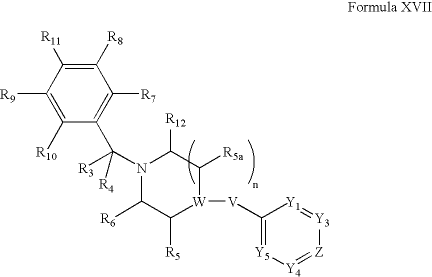

In certain aryl-substituted piperazine derivatives, R₇, R₈, R₉ and R₁₀ are each independently hydrogen, halogen, nitro, cyano, —COOH or a group of the formula M-L-; where L and M are as described above. It will be apparent that groups of the formula M-L- consist of the M component linked via the L component. If L is a single covalent bond, the group of the formula M-L- is M-.

Within further such aryl-substituted piperazine derivatives, R₇, R₈, R₉ and R₁₀ are each independently hydrogen, halogen, cyano or a group of the formula M-L-; wherein each L is independently a single covalent bond, N(R₁₃) or 0; each R₁₃ is independently hydrogen or C₁-C₆alkyl; and each M is independently hydrogen, C₁-C₆alkyl, C₂-C₆alkenyl, haloC₁-C₂alkyl or aminoC₁-C₆alkyl.

Within still further such aryl-substituted piperazine derivatives, R₇, R₈, R₉ and R₁₀ satisfy one or more of the following conditions:
R₇, R₈, R₉ and R₁₀ are each independently hydrogen, hydroxy, halogen, C₁-C₆alkyl, C₂-C₆alkenyl, C₁-C₆alkoxy, mono- or di-C₁-C₆alkylamino, haloC₁-C₂alkyl or haloC₁-C₂alkoxy.
R₇, R₈, R₉ and R₁₀ are each independently hydrogen, halogen, C₁-C₂alkyl, C₁-C₂alkoxy, haloC₁-C₂alkyl or haloC₁-C₂alkoxy.
R₁₀ is hydrogen.
R₇ and R₁₀ are hydrogen, and R₈ and R₉ are each methyl.
R₇, R₉ and R₁₀ are hydrogen, and R₈ is methyl or methoxy.
R₇ and R₈ are methyl, and R₉ and R₁₀ are both hydrogen.
   The R₁₁ Variable

In certain aryl-substituted piperazine derivatives provided herein, R₁, is a group of the formula G-L- or G-L₁-, wherein:
G is C₁-C₆alkyl, C₂-C₆alkenyl, C₂-C₆alkynyl, haloC₁-C₆alkyl, C₅-C₁₀cycloalkyl or 5- to 10-membered heterocycloalkyl, each of which is substituted with from 0 to 3 substituents independently chosen from halogen, amino and C₁-C₆alkyl, wherein G is further substituted with from 1 to 5 substituents independently chosen from Rₐ, R_{b} and R_{c};
Rₐ and R_{b} are as described above;
R_{c} is carbocycleC₀-C₆alkyl, heterocycleC₀-C₆alkyl, carbocycleC₀-C₆alkoxy, heterocycleC₀-C₆alkoxy, carbocycleC₀-C₆alkylamino or heterocycleC₀-C₆alkylamino, wherein the carbocycle is phenyl, naphthyl or C₃-C₇cycloalkyl, and the heterocycle is pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl or benzimidazolyl; each of which is substituted with from 0 to 3 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy;
L is as described above; and
L₁ is a single covalent bond, N(R₁₃), C(═O), C(═O)O, OC(═O), SO₂, SO₂N(R₁₃), N(R₁₃)SO₂, C(═O)N(R₁₃) or N(R₁₃)C(═O), wherein R₁₃ is as described above.

Within certain such aryl-substituted piperazine derivatives, G is C₁-C₆alkyl, C₂-C₆alkenyl or C₂-C₆alkynyl, each of which is substituted with from 0 to 3 substituents independently chosen from halogen and amino, and G is further substituted with from 1 to 5 substituents independently chosen from Rₐ and R_{b}.

Within other such aryl-substituted piperazine derivatives, G is C₁-C₆alkyl, C₂-C₆alkenyl or haloC₁-C₆alkyl, each of which is substituted with from 0 to 3 substituents independently chosen from halogen and amino, and G is further substituted with from 1 to 5 substituents independently chosen from Rₐ, R_{b} and R_{c}. Representative R_{c} groups include, for example, phenyl, naphthyl, C₃-C₇cycloalkyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl or benzimidazolyl, each of which is substituted with from 0 to 3 substituents independently chosen from halogen, amino, cyano, hydroxy, oxo, C₁-C₆alkyl, C₁-C₆alkoxy, (C₁-C₆alkoxy)C₁-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy.

Within certain G groups, as defined above, at least one substituent is chosen from Rₐ and R_{b}; wherein R_{b} is C₁-C₆alkoxy, (C₁-C₆alkoxy)C₁-C₆alkoxy, mono- and di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkysulfonylamino, C₁-C₆alkoxycarbonyl, C₂-C₆alkanoylamino, mono- or di-(C₁-C₆alkyl)aminocarbonyl or C₁-C₆alkyloxime, each of which is substituted with from 0 to 5 substituents independently chosen from halogen, amino, cyano, hydroxy, oxo, oxime, C₁-C₄alkyl, (C₁-C₄alkoxy)C₀-C₄alkyl, mono- and di-(C₁-C₄alkyl)amino, C₂-C₄alkanoyl, C₃-C₇cycloalkyl, C₁-C₄alkoxycarbonyl, haloC₁-C₂alkyl and haloC₁-C₂alkoxy. Within representative such compounds, G is C₁-C₆alkyl, substituted with from 0 to 3 substituents independently chosen from halogen and amino; and G is further substituted with from 1 to 5 substituents independently chosen from:
oxo, oxime, hydroxy, cyano, —(C═O)NH₂, —NH(C═O)H and imino; and
C₁-C₆alkoxy, mono- and di-(C₁-C₈alkyl)amino, C₁-C₆alkoxycarbonyl and C₂-C₆alkanoylamino, each of which is substituted with from 0 to 5 substituents independently chosen from halogen, oxo, C₁-C₄alkoxy, mono- and di-C₁-C₄alkylamino, C₂-C₄alkanoyl, C₃-C₇cycloalkyl, haloC₁-C₂alkyl and haloC₁-C₂alkoxy.

Other G groups include C₁-C₆alkyl substituted with from 0 to 2 substituents independently chosen from oxo, amino and hydroxy; each of which G is further substituted with one substituent chosen from R_{c}. Representative R_{c} groups include, for example:
heterocycloalkylC₀-C₆alkyl, heterocycloalkylC₀-C₆alkoxy or heterocycloalkylC₀-C₆alkylamino, wherein the heterocycloalkyl is pyrrolindinyl, tetrahydrofuranyl, dioxolanyl, isothiazolidinyl, piperidinyl, piperazinyl, morpholinyl or thiomorpholinyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy;
heterocycloalkylC₀-C₆alkyl, heterocycloalkylC₀-C₆alkoxy or heterocycloalkylC₀-C₆alkylamino, wherein the heterocycloalkyl is pyrrolyl, dihydropyrrolyl, pyrazolyl, imidiazolyl, triazolyl or tetrazolyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy; and
phenylC₀-C₆alkyl, phenylC₀-C₆alkoxy, phenylC₀-C₆alkylamino, pyridylC₀-C₆alkyl, pyridylC₀-C₆alkoxy, pyridylC₀-C₆alkylamino, pyrimidinylC₀-C₆alkyl, pyrimidinylC₀-C₆alkoxy or pyrimidinylC₀-C₆alkylamino, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy.

Within certain such aryl-substituted piperazine derivatives, G is C₁-C₆alkyl, C₂-C₆alkenyl, or haloC₁-C₆alkyl, each of which is substituted with from 0 to 3 substituents independently chosen from oxo, oxime, halogen, amino, hydroxy, cyano, —COOH, —(C═O)NH₂, —SO₂NH₂, —(C═N)OH, —NH(C═O)H, and imino; and G is further substituted with one substituent chosen from phenyl, naphthyl, C₃-C₇cycloalkyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl and benzimidazolyl, each of which is substituted with from 0 to 3 substituents independently chosen from halogen, amino, cyano, hydroxy, oxo, C₁-C₆alkyl, C₁-C₆alkoxy, (C₁-C₆alkoxy)C₁-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy.

In certain such compounds, G is C₁-C₆alkyl substituted with from 0 to 2 substituents independently chosen from oxo, amino and hydroxy; and G is further substituted with one substituent chosen from:
pyrrolindinyl, tetrahydrofuranyl, dioxolanyl, isothiazolidinyl, piperidinyl, piperazinyl, morpholinyl and thiomorpholinyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl, and haloC₁-C₂alkoxy;
pyrrolyl, dihydropyrrolyl, pyrazolyl, imidiazolyl, triazolyl and tetrazolyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl and haloC₁-C₂alkoxy; and
phenyl and pyridyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy.

Still further G groups include C₅-C₁₀cycloalkyl and 5- to 10-membered heterocycloalkyl, each of which is substituted with from 0 to 3 substituents independently chosen from halogen, amino and C₁-C₆alkyl, each of which G is further substituted with from 1 to 5 substituents independently chosen from Rₐ and R_{b}. Representative such G groups include, for example, C₃-C₇cycloalkyl, pyrrolindinyl, tetrahydrofuranyl, dioxolanyl, isothiazolidinyl, piperidinyl, piperazinyl, morpholinyl, and thiomorpholinyl, each of which is substituted with from 0 to 3 substituents independently chosen from halogen, amino and C₁-C₆alkyl, each of which G is further substituted with from 1 to 5 substituents independently chosen from Rₐ and R_{b}. In certain embodiments, R_{b} is C₁-C₆alkoxy, mono- and di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkysulfonylamino, C₁-C₆alkoxycarbonyl, C₂-C₆alkanoylamino, mono- or di-(C₁-C₆alkyl)aminocarbonyl or C₁-C₆alkyloxime.

In other aryl-substituted piperazine derivatives provided herein, R₁, is a group of the formula G-L- and L is O (i.e., R₁₁ is G-O—).

In still other aryl-substituted piperazine derivatives provided herein, R₁₁ is a group of the formula G-L-, and L is a single covalent bond (i.e., R₁₁ is G).

In further aryl-substituted piperazine derivatives provided herein, R₁₁ is C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy. In certain embodiments, R₁₁ is C₅-C₁₀cycloalkenyl, phenyl, naphthyl, 5- to 6-membered heterocycloalkenyl having one nitrogen ring atom and 0 or 1 additional ring heteroatoms chosen from nitrogen, oxygen and sulfur, 5- to 6-membered heteroaryl having 1, 2, 3 or 4 ring heteroatoms chosen from nitrogen, oxygen and sulfur, wherein no more than 1 ring atom is sulfur or oxygen, or 9- to 12-membered heteroaryl having 2 fused rings, wherein at least one ring is aromatic, and wherein at least one ring has 1, 2, 3 or 4 ring heteroatoms chosen from nitrogen, oxygen and sulfur, wherein no more than 3 ring atoms are sulfur or oxygen; each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl, and haloC₁-C₂alkoxy. In further embodiments, R₁₁ is C₅-C₁₀cycloalkenyl, phenyl, naphthyl, dihydropyrrolidinyl, dihydropyridinyl, tetrahydropyridinyl, furanyl, thienyl, pyrazolyl, oxazolyl, thiazolyl, thiadiazolyl, isoxazolyl, imidiazolyl, triazolyl, tetrazolyl, pyridinyl, pyrimidinyl, pyridazinyl, pyrazinyl, benzodioxanyl, indolyl, isoindolyl, indazolyl, indanyl, quinolinyl, isoquinolinyl or benzimidazolyl; each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl, and haloC₁-C₂alkoxy. In still further embodiments, R₁₁ is tetrazolyl, triazolyl, imidazolyl, or pyridinyl; each of which is substituted with from 0 to 3 substituents independently chosen from halogen, hydroxy, oxo, C₁-C₂alkyl, and C₁-C₂alkoxy, haloC₁-C₂alkyl, and haloC₁-C₂alkoxy.

In other aryl-substituted piperazine derivatives provided herein, R₁₁ is taken together with R₉ to form a fused carbocycle or heterocycle that is substituted with at least one substituent independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl, and haloC₁-C₂alkoxy. For example, in certain embodiments, R₁ is taken together with R₉ to form: (i) a fused C₅-C₇cycloalkyl or a fused phenyl; or (ii) a fused 5- to 7-membered heterocycloalkyl or 5- to 7-membered heteroaryl, each containing 1 or 2 heteroatoms independently chosen from nitrogen, oxygen, and sulfur; each of which (i) or (ii) is substituted with from 1 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl, and haloC₁-C₂alkoxy. In other embodiments, R₁₁ is taken together with R₉ to form a fused bicyclic heterocycle having one 6 membered aromatic ring and one 5-membered ring containing 1 nitrogen atom, wherein the bicyclic heterocycle is substituted with at least one substituent independently chosen from halogen, amino, cyano, hydroxy, oxo, C₁-C₆alkyl, C₁-C₆alkoxy, (C₁-C₆alkoxy)C₁-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy.

Further provided herein are aryl-substituted piperazine derivatives (e.g., of Formula I-c) in which R₁₁ is a group of the formula G₁-O—, wherein G₁ is C₂-C₆alkenyl, C₂-C₆alkynyl, haloC₁-C₆alkyl, C₃-C₁₀cycloalkyl or 4- to 10-membered heterocycloalkyl, each of which is substituted with from 0 to 3 substituents independently chosen from halogen, amino and C₁-C₆alkyl; and wherein G₁ is further substituted with from 1 to 5 substituents independently chosen from Rₐ, R_{b} and R_{c}, as defined above. In certain embodiments, one or more of the following criteria are met:

G₁ is C₂-C₆alkenyl, haloC₁-C₆alkyl, C₃-C₇cycloalkyl or a 5- to 7-membered heterocycloalkyl; each of which is substituted with from 0 to 3 substituents independently chosen from halogen, amino and haloC₁-C₂alkoxy, wherein G₁ is further substituted with from 1 to 5 substituents independently chosen from Rₐ, R_{b} and R_{c} as defined above, such that R_{c} is phenyl, naphthyl, C₃-C₇cycloalkyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl or benzimidazolyl, each of which is substituted with from 0 to 3 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy.

G₁ is C₂-C₆alkenyl, haloC₁-C₆alkyl, C₃-C₇cycloalkyl or a 5- to 7-membered heterocycloalkyl; each of which is substituted with from 0 to 3 substituents independently chosen from halogen, amino and haloC₁-C₂alkoxy, wherein G₁ is further substituted with from 1 to 5 substituents independently chosen from: (a) oxo, hydroxy, cyano, —(C═O)NH₂, —NH(C═O)H and imino; and (b) C₁-C₆alkoxy, mono- and di-(C₁-C₈alkyl)amino, C₁-C₆alkoxycarbonyl, and C₂-C₆alkanoylamino, each of which is substituted with from 0 to 5 substituents independently chosen from halogen, oxo, C₁-C₄alkoxy, mono- and di-C₁-C₄alkylamino, C₂-C₄alkanoyl, C₃-C₇cycloalkyl, haloC₁-C₂alkyl and haloC₁-C₂alkoxy.

G₁ is C₂-C₆alkenyl, haloC₁-C₆alkyl, a C₃-C₇cycloalkyl or a 5- to 7-membered heterocycloalkyl; each of which is substituted with from 0 to 2 substituents independently chosen from oxo, amino and hydroxy; wherein G₁ is further substituted with one substituent chosen from phenyl, naphthyl, C₃-C₇cycloalkyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl and benzimidazolyl, each of which is substituted with from 0 to 3 substituents independently chosen from halogen, amino, cyano, hydroxy, oxo, C₁-C₆alkyl, C₁-C₆alkoxy, (C₁-C₆alkoxy)C₁-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy.

G₁ is C₂-C₆alkenyl, haloC₁-C₆alkyl, a C₃-C₇cycloalkyl, or a 5- to 7-membered heterocycloalkyl; each of which is substituted with from 0 to 2 substituents independently chosen from oxo, amino and hydroxy; wherein G₁ is further substituted with one substituent chosen from pyrrolindinyl, tetrahydrofuranyl, dioxolanyl, isothiazolidinyl, piperidinyl, piperazinyl, morpholinyl and thiomorpholinyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl and haloC₁-C₂alkoxy.

G₁ is C₂-C₆alkenyl, haloC₁-C₆alkyl, C₃-C₇cycloalkyl or a 5- to 7-membered heterocycloalkyl; each of which is substituted with from 0 to 2 substituents independently chosen from oxo, amino and hydroxy; wherein G₁ is further substituted with one substituent chosen from pyrrolyl, dihydropyrrolyl, pyrazolyl, imidiazolyl, triazolyl and tetrazolyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl and haloC₁-C₂alkoxy.

G₁ is C₂-C₆alkenyl, haloC₁-C₆alkyl, a C₃-C₇cycloalkyl or a 5- to 7-membered heterocycloalkyl; each of which is substituted with from 0 to 2 substituents independently chosen from oxo, amino and hydroxy; wherein G₁ is further substituted with one substituent chosen from phenyl and pyridyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy.

In yet other aryl-substituted piperazine derivatives provided herein (e.g., those of Formula I-c), R₁₁ is a group of the formula G₂-O— in which G₂ is C₁-C₆alkyl that is substituted with from 0 to 3 substituents independently chosen from halogen and amino, wherein G₂ is further substituted with from 1 to 5 substituents independently chosen from Rₐ, R_{b} and R_{c}, as described above, such that R_{b} is not N-methyl,N-cyclopentylamino. In certain embodiments, one or more of the following criteria are met:

R_{c} is not (heterocycle)C₀-C₆alkyl.

R_{c} is phenyl, naphthyl, C₃-C₇cycloalkyl, C₃-C₇cycloalkenyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl or benzimidazolyl, each of which is substituted with from 0 to 3 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl, and haloC₁-C₂alkoxy.

G₂ is substituted with from 1 to 5 substituents independently chosen from (a) oxo, hydroxy, cyano, —(C═O)NH₂, —NH(C═O)H and imino; and (b) C₁-C₆alkoxy, mono- and di-(C₁-C₈alkyl)amino, C₁-C₆alkoxycarbonyl and C₂-C₆alkanoylamino, each of which is substituted with from 0 to 5 substituents independently chosen from halogen, oxo, C₁-C₄alkoxy, mono- and di-C₁-C₄alkylamino, C₂-C₄alkanoyl, C₃-C₇cycloalkyl, haloC₁-C₂alkyl and haloC₁-C₂alkoxy.

G₂ is substituted with at least one substituent chosen from phenyl, naphthyl, C₃-C₇cycloalkyl, pyrrolidinyl, tetrahydrofuranyl, dioxolanyl, tetrahydropyranyl, isothiazolidinyl, piperidinyl, piperazinyl, morpholinyl, thiomorpholinyl, pyrrolyl, dihydropyrrolyl, furanyl, thienyl, pyrazolyl, oxazolyl, thiazolyl, thiadiazolyl, isoxazolyl, imidiazolyl, triazolyl, tetrazolyl, pyridinyl, tetrahydropyridinyl, pyrimidinyl, pyridazinyl, pyrazinyl, benzodioxanyl, indolyl, isoindolyl, indazolyl, indanyl, quinolinyl, isoquinolinyl and benzimidazolyl, each of which is substituted with from 0 to 3 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, C₃-C₇cycloalkyl, C₁-C₄alkoxycarbonyl, haloC₁-C₂alkyl and haloC₁-C₂alkoxy. In certain embodiments, G₂ is substituted with exactly one such substituent.

G₂ is substituted with at least one substituent chosen from pyrrolindinyl, tetrahydrofuranyl, dioxolanyl, isothiazolidinyl, piperidinyl, piperazinyl, morpholinyl and thiomorpholinyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl and haloC₁-C₂alkoxy. In certain embodiments, G₂ is substituted with exactly one such substituent.

G₂ is substituted with at least one substituent chosen from pyrrolyl, dihydropyrrolyl, pyrazolyl, imidiazolyl, triazolyl and tetrazolyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl, haloC₁-C₂alkyl and haloC₁-C₂alkoxy. In certain embodiments, G₂ is substituted with exactly one such substituent.

G₂ is substituted with at least one substituent chosen from phenyl and pyridyl, each of which is substituted with from 0 to 3 substituents independently chosen from halogen, amino, cyano, hydroxy, oxo, C₁-C₄alkyl, C₁-C₄alkoxy, mono- and di-C₁-C₄alkylamino, C₂-C₄alkanoyl and haloC₁-C₂alkoxy. In certain embodiments, G₂ is substituted with exactly one such substituent.

Further provided herein are aryl-substituted piperazine derivatives in which R₁₁ is a group of formula M-L- or M-L₁-. In certain embodiments L is O; in other embodiments L is a single covalent bond. In certain such aryl-substituted piperazine derivatives, M is a 5- to 10-membered cycloalkyl or heterocycloalkyl. For example, in some embodiments M is C₃-C₇cycloalkyl, pyrrolindinyl, tetrahydrofuranyl, dioxolanyl, isothiazolidinyl, piperidinyl, piperazinyl, morpholinyl or thiomorpholinyl. In other embodiments, M is C₁-C₆alkyl, C₂-C₆alkenyl, C₂-C₆alkynyl, haloC₁-C₆alkyl or aminoC₁-C₆alkyl.

Also provided herein are aryl-substituted piperazine derivatives of Formulas XVIII-XXI:

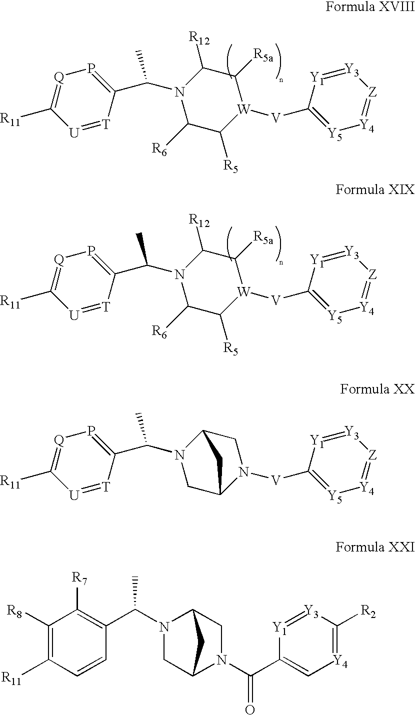

With Formula XVIII-Formula XXI, the variables n, R₅, R₆, R₁₁, R₁₂ Y₁, Y₃, Y₄, Y₅, P, Q, U, T, W and Z carry any of the values set forth above.

Within certain aryl-substituted piperazine derivatives of Formula XXI:
each R₁ is hydrogen or methoxy;
R₂ is chloro, fluoro or trifluoromethyl;
R₇ and R₈ are independently hydrogen, halogen, hydroxy, nitro, cyano, —COOH or a group of the formula M-L-; and
R₁₁ is:
a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ, R_{b} and R_{c}; or
C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇Cycloalkyl)C₀-C₆alkyl, C₁-C₄alkoxycarbonyl, haloC₁-C₆alkyl and haloC₁-C₆alkoxy.

Within further such aryl-substituted piperazine derivatives of Formula XXI:
R₇ and R₈ are independently hydrogen, halogen, C₁-C₂alkyl or haloC₁-C₂alkyl; and
R₁₁ is a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ and R_{b}.

Still further such compounds satisfy one of the following criteria:
Y₁ is N and Y₃ and Y₄ are CR₁.
Y₃ and Y₄ are CR₁ (e.g., CH).
Y₃ is N.
Y₃ and Y₄ are N.
Y₁, Y₃ and Y₄ are N.
Y₁ and Y₃ are N, and Y₄ is CR₁.

In yet another embodiment, aryl-substituted piperazine derivatives of Formula XXII are provided:

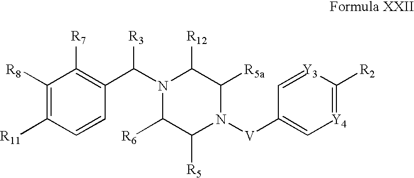

Within Formula XXII:
each R₁ is hydrogen or methoxy;
R₂ is chloro, fluoro or trifluoromethyl;
R₃ is:
hydrogen or methyl; or
taken together with R₆ to form a fused 5- to 7-membered heterocycloalkyl that has 0 or 1 additional heteroatoms chosen from N, S and O, which fused 5- to 7-membered heterocycloalkyl is substituted with from 0 to 2 substituents independently chosen from halogen, oxo, C₁-C₂alkoxy and C₁-C₂alkyl;
R₅ is hydrogen, methyl or methoxy;
R₅ₐ is:
hydrogen, methyl or methoxy;
taken together with R₆ to form a methylene or ethylene bridge;
R₆ is:
hydrogen, methyl, or methoxy;
taken together with R₃ to form a fused, optionally substituted, 5- to 7-membered heterocycloalkyl; or
taken together with R₅ₐ to form a methylene or ethylene bridge;
R₇ and R₈ are independently hydrogen, halogen, hydroxy, nitro, cyano, —COOH or a group of the formula M-L-;
R₁₁ is:
a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ, R_{b} and R_{c}; or
C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy; and
R₁₂ is hydrogen, methyl or methoxy.

Further such aryl-substituted piperazine derivatives of Formula XXIII satisfy Formula XXIII: 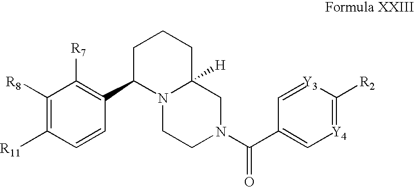
wherein:
R₇ and R₈ are independently hydrogen, halogen, C₁-C₂alkyl or haloC₁-C₂alkyl; and
R₁₁ is a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ and R_{b}.

Still further such compounds satisfy one of the following criteria:
Y₁ is N and Y₃ and Y₄ are CR₁.
Y₃ and Y₄ are CR₁ (e.g., CH).
Y₃ is N and Y₄ is CR₁ (e.g., CH).
Y₃ and Y₄ are N.

Representative aryl-substituted piperazine derivatives of Formulas I-XXIII include, but are not limited to, those specifically described in Examples 1-31. It will be apparent that the compounds recited therein are representative only, and are not intended to limit the scope of the present invention. Further, as noted above, all compounds may be present as a free base, a pharmaceutically acceptable salt (such as an acid addition salt) or other form, such as a hydrate.

In certain embodiments, aryl-substituted piperazine derivatives provided herein detectably alter (modulate) MCH binding to MCH1R and/or MCH2R, as determined using a standard in vitro MCH receptor ligand binding assay and/or functional assay. References herein to a “MCH receptor ligand binding assay” refer to either of the assays provided in Examples 33 and 36. Within such assays, the receptor is incubated with labeled MCH (or other suitable ligand) and a test compound. A test compound that detectably modulates binding of ligand to MCH receptor will result in a decrease or increase in the amount of label bound to the MCH receptor preparation, relative to the amount of label bound in the absence of the compound. Preferably, such a compound will exhibit a Kᵢ at an MCH receptor that is less than 1 micromolar, more preferably less than 500 nM, 100 nM, 20 nM or 10 nM, within an assay performed as described in Example 33 and/or within an assay performed as described in Example 36. Certain preferred compounds are MCH receptor antagonists, and exhibit IC₅₀ values of about 4 micromolar or less, more preferably 1 micromolar or less, still more preferably about 100 nanomolar or less, or 10 nanomolar or less within a standard in vitro MCH receptor mediated calcium mobilization assay, as provided in Example 37 and/or an agonist-stimulated GTP gamma³⁵S binding assay, as described in Example 35.

If desired, aryl-substituted piperazine derivatives provided herein may be evaluated for certain pharmacological properties including, but not limited to, oral bioavailability (preferred compounds are orally bioavailable to an extent allowing for oral doses of less than 140 mg/kg, preferably less than 50 mg/kg, more preferably less than 30 mg/kg, even more preferably less than 10 mg/kg, still more preferably less than 1 mg/kg), toxicity (a preferred compound is nontoxic when a therapeutically effective amount is administered to a subject), side effects (a preferred compound produces side effects comparable to placebo when a therapeutically effective amount of the compound is administered to a subject), serum protein binding and in vitro and in vivo half-life (a preferred compound exhibits an in vitro half-life that is equal to an in vivo half-life allowing for Q.I.D. dosing, preferably T.I.D. dosing, more preferably B.I.D. dosing, and most preferably once-a-day dosing). In addition, differential penetration of the blood brain barrier may be desirable for compounds used to treat CNS disorders, while low brain levels of compounds used to treat peripheral disorders are preferred. Routine assays that are well known in the art may be used to assess these properties and identify superior compounds for a particular use. For example, assays used to predict bioavailability include transport across human intestinal cell monolayers, including Caco-2 cell monolayers. Penetration of the blood brain barrier of a compound in humans may be predicted from the brain levels of the compound in laboratory animals given the compound (e.g., intravenously). Serum protein binding may be predicted from albumin binding assays. Compound half-life is inversely proportional to the frequency of dosage of a compound. In vitro half-lives of compounds may be predicted from assays of microsomal half-life as described in Example 39.

As noted above, preferred aryl-substituted piperazine derivatives provided herein are nontoxic. In general, the term “nontoxic” shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria when administered in minimum therapeutically effective amounts, or when contacted with cells at a concentration that is sufficient to inhibit the binding of ligand to MCH receptor in vitro: (1) does not substantially inhibit cellular ATP production; (2) does not significantly prolong heart QT intervals; (3) does not cause substantial liver enlargement and (4) does not cause substantial release of liver enzymes.

As used herein, a compound that does not substantially inhibit cellular ATP production is a compound that satisfies the criteria set forth in Example 38. In other words, cells treated as described in Example 38 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells. The concentration of compound used in such assays is generally at least 10-fold, 100-fold or 1000-fold greater than the EC₅₀ or IC₅₀ for the modulator in the assay of Example 35 or 37.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05, 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₋₆ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of twice the minimum dose that yields a serum concentration equal to the EC₅₀ or IC₅₀ for the compound does not elevate serum levels of ALT, LDH or AST in laboratory rodents by more than 100% over matched mock-treated controls. In more preferred embodiments, such doses do not elevate such serum levels by more than 75% or 50% over matched controls. Alternatively, a compound does not promote substantial release of liver enzymes if, in an in vitro hepatocyte assay, concentrations (in culture media or other such solutions that are contacted and incubated with hepatocytes in vitro) that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold, the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic (e.g., as determined using a mouse erythrocyte precursor cell micronucleus assay, an Ames micronucleus assay, a spiral micronucleus assay or the like) at a concentration equal the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, aryl-substituted piperazine derivatives provided herein may be isotopically-labeled or radiolabeled. For example, compounds of Formula I may have one or more atoms replaced by an atom of the same element having an atomic mass or mass number different from the atomic mass or mass number usually found in nature. Examples of isotopes that can be present in the compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, such as increased in vivo half-life or reduced dosage requirements and, hence, may be preferred in some circumstances.

Pharmaceutical Compositions

Aryl-substituted piperazine derivatives can be administered as the neat chemical, but are preferably administered as a pharmaceutical composition comprising such a compound, together with at least one physiologically acceptable carrier or excipient. Representative carriers include, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol and proteins. Additional optional components include, adjuvants, diluents, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs).

Pharmaceutical carriers must be of sufficiently high purity and sufficiently low toxicity to render them suitable for administration to the animal being treated. The carrier can be inert or it can possess pharmaceutical benefits. The amount of carrier employed in conjunction with the compound is sufficient to provide a practical quantity of material for administration per unit dose of the compound. Representative pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil and corn oil; polyols such as propylene glycol, glycerine, sorbitol, mannitol and polyethylene glycol; alginic acid; phosphate buffer solutions; emulsifiers, such as the TWEENS; wetting agents, such as sodium lauryl sulfate; coloring agents; flavoring agents; tableting agents; stabilizers; antioxidants; preservatives; pyrogen-free water; isotonic saline; and phosphate buffer solutions.

To prepare a pharmaceutical composition, effective concentrations of one or more aryl-substituted piperazine derivatives provided herein are mixed with one or more a suitable pharmaceutical carriers or excipients. In instances in which the compounds exhibit insufficient solubility, methods for solubilizing compounds may be used. Such methods are known to those of skill in this art and include, but are not limited to, using cosolvents such as dimethylsulfoxide (DMSO), using surfactant, such as TWEEN, or dissolution in aqueous sodium bicarbonate. Upon mixing or addition of the compound(s), the resulting mixture may be a solution, suspension, emulsion or the like. The form of the resulting mixture depends upon a number of factors, including the intended mode of administration and the solubility of the compound in the chosen carrier.

Pharmaceutical compositions may be formulated for administration by any suitable route, including orally, topically, parenterally, by inhalation or spray, sublingually, transdermally, via buccal administration, rectally, as an ophthalmic solution or by other means, and may be prepared in dosage unit formulations. Dosage formulations suitable for oral use include, for example, tablets, troches, lozenges, liquid solutions, aqueous or oily suspensions, dispersible powders or granules, emulsions, hard or soft capsules, tinctures, syrups or elixirs. Compositions intended for oral use may further contain one or more optional agents, such as sweetening agents (e.g., glycerol, propylene glycol, sorbitol or sucrose), flavoring agents, coloring agents and preserving agents, in order to provide pharmaceutically appealing and palatable preparations. Such formulations may also contain a demulcent. Typical components of carriers for syrups, elixirs, emulsions and suspensions include ethanol, glycerol, propylene glycol, polyethylene glycol, liquid sucrose, sorbitol and water.

Orally Administered Liquid Formulations

Compounds provided herein can be incorporated into oral liquid preparations such as, for example, aqueous or oily suspensions, solutions, emulsions, syrups or elixirs. Moreover, formulations containing these compounds can be presented as a dry product for constitution with water or other suitable vehicle before use. Such liquid preparations may further contain one or more conventional additives, such as suspending agents (e.g., sorbitol syrup, methyl cellulose, glucose/sugar, syrup, gelatin, hydroxyethyl cellulose, carboxymethyl cellulose, aluminum stearate gel and hydrogenated edible fats); emulsifying agents (e.g., lecithin, sorbitan monsoleate or acacia); and/or non-aqueous vehicles such as edible oils (e.g., almond oil, fractionated coconut oil, silyl esters, propylene glycol and ethyl alcohol) and preservatives (e.g., methyl or propyl p-hydroxybenzoate and sorbic acid).

Suspensions

Aqueous suspensions contain the active material(s) in admixture with excipients (e.g., suspending agents, wetting agents and/or preservatives) suitable for the manufacture of aqueous suspensions. Suspending agents include, for example, sodium carboxymethylcellulose, methylcellulose, hydropropylmethylcellulose, AVICEL RC-591, sodium alginate, polyvinylpyrrolidone, gum tragacanth and gum acacia. Dispersing or wetting agents include, for example, lecithin, polysorbate 80, naturally-occurring phosphatides such as lecithin, condensation products of an alkylene oxide with fatty acids (e.g., polyoxyethylene stearate), condensation products of ethylene oxide with long chain aliphatic alcohols (e.g., heptadecaethyleneoxycetanol), condensation products of ethylene oxide with partial esters derived from fatty acids and a hexitol (e.g., polyoxyethylene sorbitol substitute), or condensation products of ethylene oxide with partial esters derived from fatty acids and hexitol anhydrides (e.g., polyethylene sorbitan substitute). Representative preservatives include, for example, ethyl- or n-propyl-p-hydroxybenzoate, sodium benzoate and methyl paraben.

Oily suspensions may be formulated by suspending the active ingredients in a vegetable oil (e.g., peanut oil, olive oil, sesame oil or coconut oil), a mineral oil (such as liquid paraffin) or a mixture of such oils. The oily suspensions may further contain a thickening agent, such as beeswax, hard paraffin or cetyl alcohol. Sweetening agents, such as those set forth above, and flavoring agents may be added to improve palatability. If desired, these compositions may be preserved by the addition of an anti-oxidant such as ascorbic acid.

Emulsions

Pharmaceutical compositions provided herein may also be in the form of oil-in-water emulsions. The oily phase may be a vegetable oil, mineral oil, or mixture thereof as described above. Suitable emulsifying agents include naturally-occurring gums (e.g., gum acacia or gum tragacanth), naturally-occurring phosphatides (e.g., soy bean phosphatide, lecithin and esters or partial esters derived from fatty acids and hexitol), and anhydrides (e.g., sorbitan monoleate and condensation products of the above partial esters with ethylene oxide, such as polyoxyethylene sorbitan monoleate).

Dispersible Powders

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets and Capsules

Tablets typically comprise conventional pharmaceutically compatible inert diluents, such as calcium carbonate, sodium carbonate, mannitol, lactose and cellulose; binders such as starch, gelatin and sucrose; disintegrants such as starch, alginic acid and croscarmelose; and/or lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint and fruit flavors, are useful adjuvants for chewable tablets. Capsules (including time release and sustained release formulations) typically comprise one or more solid diluents disclosed above. The selection of carrier components often depends on secondary considerations such as taste, cost and shelf stability.

Such compositions may also be coated by conventional methods, typically with pH-dependent or time-dependent coatings, such that the subject compound is released in the gastrointestinal tract in the vicinity of the desired topical application, or at various times to extend the desired action. Such coatings typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, such as calcium carbonate, calcium phosphate or kaolin, or as soft gelatin capsules wherein the active ingredient is mixed with water or an oil medium, such as peanut oil, liquid paraffin or olive oil.

Injectable and Parenteral Formulations

Pharmaceutical compositions may be in the form of a sterile injectable aqueous or oleaginous suspension. Such a suspension may be formulated according to the known art using dispersing or wetting agents and suspending agents as described above. The sterile injectable preparation may also be a sterile injectable solution or suspension in a non-toxic parentally acceptable diluent or solvent (e.g., as a solution in 1,3-butanediol). Among the acceptable vehicles and solvents that may be employed are water, Ringer's solution and isotonic sodium chloride solution. In addition, sterile, fixed oils are conventionally employed as a solvent or suspending medium. For this purpose any bland fixed oil synthetic (e.g., synthetic mono- or diglycerides) may be employed. In addition, fatty acids such as oleic acid are useful in the preparation of injectable formulations.

Pharmaceutical compositions may be administered parenterally in a sterile medium. Parenteral administration includes subcutaneous injections, intravenous, intramuscular, intrathecal injection or infusion techniques. The active agent(s), depending on the vehicle and concentration used, can either be suspended or dissolved in the vehicle. Adjuvants such as local anesthetics, preservatives and buffering agents can also be dissolved in the vehicle. In many compositions for parenteral administration, at least about 90% by weight of the total composition is carrier. Preferred carriers for parenteral administration include propylene glycol, ethyl oleate, pyrrolidone, ethanol and sesame oil.

Suppositories

Pharmaceutical compositions may also be administered rectally, in the form of suppositories. Such compositions can be prepared by mixing the active ingredient(s) with a suitable non-irritating excipient that is solid at ordinary temperatures but liquid at rectal temperature and will therefore melt in the rectum to release the drug. Such materials include cocoa butter and polyethylene glycols.

Topical Formulations

Pharmaceutical compositions may be formulated for local or topical application, such as for topical application to the skin or mucous membranes. Topical compositions may be in any suitable form including, for example, solutions, creams, ointments, gels, lotions, milks, cleansers, moisturizers, sprays, skin patches and the like. Such solutions may, for example, be formulated as 0.01%-10% isotonic solutions, pH about 5-7, with appropriate salts. Pharmaceutical compositions may also be formulated for transdermal administration as a transdermal patch.

Topical compositions containing the active compound can be admixed with a variety of carrier materials well known in the art, such as, for example, water, alcohols, aloe vera gel, allantoin, glycerine, vitamin A and E oils, mineral oil, propylene glycol, PPG-2 myristyl propionate and the like. Other materials suitable for use in topical carriers include, for example, emollients, solvents, humectants, thickeners and powders. Examples of each of these types of materials, which can be used singly or as mixtures of one or more materials, are as follows: emollients, such as stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate and myristyl myristate; propellants, such as propane, butane, iso-butane, dimethyl ether, carbon dioxide and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

Pharmaceutical compositions may also be topically administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Other Formulations and Additional Components

Other compositions useful for attaining systemic delivery of the subject compounds include sublingual, buccal and nasal dosage forms. Such compositions typically comprise one or more soluble filler substances such as sucrose, sorbitol and mannitol, and/or binders such as acacia, microcrystalline cellulose, carboxymethyl cellulose and hydroxypropyl methylcellulose. Glidants, lubricants, sweeteners, colorants, antioxidants and flavoring agents disclosed above may also be included.

Compositions for inhalation are typically provided in the form of a solution, suspension or emulsion that can be administered as a dry powder or in the form of an aerosol using a conventional propellant (e.g., dichlorodifluoromethane or trichlorofluoromethane).

In addition to or together with the above modes of administration, a pharmaceutical composition may be conveniently added to food or drinking water (e.g., for administration to non-human animals including companion animals, such as dogs and cats and livestock). Animal feed and drinking water compositions may be formulated so that the animal takes in an appropriate quantity of the composition along with its diet. It may also be convenient to present the composition as a premix for addition to feed or drinking water.

Pharmaceutical compositions may also optionally comprise an activity enhancer. The activity enhancer can be chosen from a wide variety of molecules that function in different ways to enhance MCH receptor modulator effect. Particular classes of activity enhancers include skin penetration enhancers and absorption enhancers.

Pharmaceutical Compositions for Combination Therapy

Pharmaceutical compositions provided herein may also contain additional active agents, which can be chosen from a wide variety of molecules and can function in different ways to enhance the therapeutic effects of a MCH receptor modulator, or to provide a separate therapeutic effect that does not substantially interfere with the activity of the MCH receptor modulator. Such optional active agents, when present, are typically employed in the compositions described herein at a level ranging from about 0.01% to about 50% by weight of the composition, preferably 0.1% to 25%, 0.2% to 15, 0.5% to 10% or 0.5% to 5% by weight of the composition. For example, compositions intended for the treatment of obesity and/or eating disorders, such as bulimia nervosa, may further comprise leptin, a leptin receptor agonist, a melanocortin receptor 4 (MC4) agonist, sibutramine, dexfenfluramine, a growth hormone secretagogue, a beta-3 agonist, a 5HT-2 agonist, an orexin antagonist, a neuropeptide Y₁ or Y₅ antagonist, a galanin antagonist, a CCK agonist, a GLP-1 agonist, a cannabinoid receptor antagonist (e.g., a CB1 antagonist) and/or a corticotropin-releasing hormone agonist. Other active ingredients that may be included within the compositions provided herein include antidepressants, inhibitors of dipeptidyl peptidase IV (DPP IV) and/or diuretics.

In certain embodiments, an additional active agent is a CB1 antagonist. Representative CB1 antagonists include, for example, certain pyrimidines (e.g., PCT International Application Publication No. WO 04/029,204), pyrazines (e.g., PCT International Application Publication Nos. WO 01/111,038; WO 04/111,034 and WO 04/111,033), azetidine derivatives (e.g., U.S. Pat. Nos. 6,518,264; 6,479,479 and 6,355,631; and PCT International Application Publication No. WO 03/053431), pyrazole derivatives (e.g., U.S. Pat. Nos. 6,509,367 and 6,476,060; and PCT International Application Publication Nos. WO 03/020217 and WO 01/029007), pyrazolecarboxylic acid and pyrazole carboxamide derivatives (e.g., U.S. Pat. Nos. 6,645,985; 6,432,984; 6,344,474; 6,028,084; 5,925,768; 5,624,941 and 5,462,960; published U.S. applications U.S. 2004/0039024; U.S. 2003/0199536 and U.S. 2003/0003145; and PCT International Application Publication Nos. WO 03/078413; WO 03/027076; WO 03/026648 and WO 03/026647); aroyl substituted benzofurans (e.g., LY-320135, U.S. Pat. No. 5,747,524); substituted imidazoles (e.g., published U.S. application U.S. 2003/0114495 and PCT International Application Publication Nos. WO 03/063781 and WO 03/040107); substituted furo[2,3-b]pyridine derivatives (e.g., PCT International Application Publication No. WO 04/012671); substituted aryl amides (e.g., PCT International Application Publication Nos. WO 03/087037 and WO 03/077847); substituted bicyclic or spirocyclic amides (e.g., PCT International Application Publication Nos. WO 03/086288 and WO 03/082190); and substituted 2,3-diphenyl pyridines (e.g., PCT International Application Publication No. WO 03/082191). Other CB1 antagonists are cannabidiol and its derivatives. Preferred CB1 antagonists include, for example, aryl substituted pyrazole carboxamides such as SR-141716A (N-piperidin-1-yl)-5-(4-chlorophenyl)-1-(2,4-dichlorophenyl)-4-methyl-1-H-pyrazole-3-carboxamide, also known as RIMONABAN™ or ACOMPLIA™) as well analogues thereof such as AM251 (N-piperidin-1-yl)-5-(4-iodophenyl)-1-(2,4-dichlorophenyl)-4-methyl-1-H-pyrazole-3-carboxamide) and AM281 (N-(morpholin-4-yl)-1-(2,4-dichlorophenyl)-5-(4-iodophenyl)-4-methyl-1-H-pyrazole-3-carboxamide); various azetidine compounds (e.g., U.S. Pat. Nos. 6,518,264; 6,479,479 and 6,355,631) and the imidazoles 1-(4-chlorophenyl)-2-(2-chlorophenyl)-N-[(1S,2S)-2-hydroxycyclohexyl]-1H-imidazole-4-carboxamide and 2-(2-chlorophenyl)-1-(4-chlorophenyl)-N′-[4-(trifluoromethyl)phenyl]-1H-imidazole-4-carbohydrazide.

Packaged Pharmaceutical Preparations

Pharmaceutical compositions may be packaged for treating or preventing a disease or disorder that is associated with MCH receptor activation (e.g., treatment of metabolic disorders such as diabetes, heart disease, stroke, obesity and eating disorders such as bulimia, skin disorders such as vitiligo, or sexual disorders such as anorgasmic or psychogenic impotence), or for promoting weight loss. Packaged pharmaceutical preparations comprise a container holding a therapeutically effective amount of MCH receptor modulator as described herein and instructions (e.g., labeling) indicating that the contained composition is to be used for promoting weight loss or for treating or preventing a disease or disorder that is associated with MCH receptor activation in the patient. Prescribing information may be provided separately to a patient or health care provider, or may be provided as a label or package insert. Prescribing information may include, for example, efficacy, dosage and administration, contraindication and adverse reaction information pertaining to the pharmaceutical formulation. Certain packaged pharmaceutical preparations further include a second therapeutic agent as discussed above.

Dosages

Aryl-substituted piperazine derivatives are generally present within a pharmaceutical composition in a therapeutically effective amount. Compositions providing dosage levels ranging from about 0.1 mg to about 140 mg per kilogram of body weight per day are preferred (about 0.5 mg to about 7 g per human patient per day), with dosages ranging from 0.1 mg to 50 mg, 30 mg or 10 mg particularly preferred. The amount of active ingredient that may be combined with the carrier to produce a single dosage form will vary depending upon the patient to be treated and the particular mode of administration. Dosage unit forms generally contain from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Dosage units generally contain from about 10 μg to about 500 mg of each active ingredient. Optimal dosages may be established using routine testing and procedures that are well known in the art.

Methods of Use

Within certain aspects, the present invention provides methods for inhibiting the development or progression of a disease or disorder responsive to MCH receptor modulation. In other words, therapeutic methods provided herein may be used to treat a patient already afflicted with such a disease or disorder, or may be used to prevent or delay the onset of such a disease or disorder in a patient who is free of detectable disease or disorder that is associated with MCH receptor activation. As noted above, a disease or disorder is “associated with MCH receptor activation” if it is characterized by inappropriate stimulation of MCH receptor, regardless of the amount of MCH present locally, and/or is responsive to modulation of MCH receptor activity. Such conditions include, for example, metabolic disorders (such as diabetes), heart disease, stroke, eating disorders (such as obesity and bulimia nervosa), disorders of the skin such as vitiligo, and sexual disorders such as anorgasmic or psychogenic impotence. These conditions may be diagnosed and monitored using criteria that have been established in the art. In addition, MCH antagonists provided herein may be used to promote weight loss in patients, and MCH agonists provided herein may be used to promote weight gain in patients. Patients may include humans, domesticated companion animals (pets, such as dogs and cats) and livestock animals, with dosages and treatment regimes as described above.

Additional conditions that are associated with MCH receptor activation include:

Cognitive impairment and memory disorders, such as Alzheimer's disease, Parkinson's disease, mild cognitive impairment (MCI), age-related cognitive decline (ARCD), stroke, traumatic brain injury, AIDS associated dementia, and dementia associated with depression, anxiety and psychosis (including schizophrenia and hallucinatory disorders);

Anxiety, depression and other mood disorders, including general anxiety disorder (GAD), agoraphobia, panic disorder with and without agoraphobia, social phobia, specific phobia, post traumatic stress disorder, obsessive compulsive disorder (OCD), dysthymia, adjustment disorders with disturbance of mood and anxiety, separation anxiety disorder, anticipatory anxiety acute stress disorder, adjustment disorders and cyclothymia;

Reward system disorders such as addiction (e.g., opioid, nicotine or alcohol);

Pain such as migraine, peripheral inflammatory pain, neuropathic pain and sympathetic nervous system associated pain; and

Peripheral indications such as respiratory disorders (e.g., asthma), urinary disorders (e.g., urinary incontinence), gastrointestinal disorders, reproductive function disorders and cardiovascular disorders (e.g., arteriosclerosis and hypertension).

Frequency of dosage may vary depending on the compound used and the particular disease to be treated or prevented. In general, for treatment of most disorders, a dosage regimen of 4 times daily or less is preferred. For the treatment of eating disorders and obesity, a dosage regimen of 1 or 2 times daily is particularly preferred. For the treatment of impotence a single dose that rapidly reaches effective concentrations is desirable. It will be understood, however, that the specific dose level for any particular patient will depend upon a variety of factors including the activity of the specific compound employed, the patient's age, body weight, general health, sex and diet, the time and route of administration, the rate of excretion, any coadministered drugs and the severity of the particular disease. In certain embodiments, administration at meal times is preferred. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

In other aspects, methods for treating a patient are provided, comprising diagnosing the patient as having a disease or disorder associated with MCH receptor activation, correlating the diagnosis of the disease or disorder with the need for MCH modulator administration, and administering an a effective amount of an aryl-substituted piperazine derivative provided herein. A method for treating a patient comprising administering an effective amount of an aryl-substituted piperazine derivative of Formula I to a patient having a disease or disorder associated with MCH receptor activation is also provided herein.

Within certain embodiments the disease or disorder associated with MCH receptor activation is obesity, an eating disorder, a sexual disorder, diabetes, heart disease or stroke.

Within certain embodiments provided herein the aryl-substituted piperazine derivative of Formula I is administered orally, intranasally, intravenously or topically.

Within certain aspects, MCH receptor modulators provided herein may be used within combination therapy for the treatment of conditions associated with MCH receptor modulation. Within combination therapy, a MCH receptor modulator is administered to a patient along with a second therapeutic agent that is not primarily a MCH receptor modulator, but that is appropriate for treatment of the condition(s) of interest. The MCH receptor modulator and second therapeutic agent(s) may be present in the same pharmaceutical composition, or may be administered separately in either order. Suitable second therapeutic agents include those listed above.

Suitable dosages for MCH receptor modulator(s) within such combination therapy are generally as described herein. Dosages and methods of administration of other therapeutic agents can be found, for example, in the manufacturer's instructions in the Physician's Desk Reference. In certain embodiments, the combination administration results in a reduction of the dosage of the second therapeutic agent required to produce a therapeutic effect (i.e., a decrease in the minimum therapeutically effective amount). Thus, preferably, the dosage of second therapeutic agent in a combination or combination treatment method of the invention is less than the maximum dose advised by the manufacturer for administration of the second therapeutic agent without combination administration of a MCH receptor modulator. More preferably this dosage is less than 3, even more preferably less than ½, and highly preferably, less than ¼ of the maximum dose, while most preferably the dose is less than 10% of the maximum dose advised by the manufacturer for administration of the second therapeutic agent(s) when administered without combination administration of a MCH receptor modulator. It will be apparent that the dosage amount of MCH receptor modulator component of the combination needed to achieve the desired effect may similarly be affected by the dosage amount and potency of the second therapeutic agent component of the combination.

In certain preferred embodiments, the combination administration of a MCH receptor modulator with a second therapeutic agent is accomplished by packaging one or more MCH receptor modulators and one or more second therapeutic agents in the same package, either in separate containers within the package or in the same container as a mixture of one or more MCH receptor modulators and one or more second therapeutic agents. Preferred mixtures are formulated for oral administration (e.g., as pills, capsules, tablets or the like). In certain embodiments, the package comprises a label or package insert indicating that the one or more MCH receptor modulators and one or more second therapeutic agents are to be taken together for the treatment of a condition that is associated with MCH receptor activation, such as obesity.

In certain embodiments, one or more MCH receptor modulators provided herein are used along with one or more CB1 antagonists within a combination therapy. Such combinations are of particular use for weight management, to reduce appetite and/or food intake or to prevent or treat obesity (e.g., promote weight loss). Patients may include humans, domesticated companion animals and livestock animals, with dosages and treatment regimes as described above. The MCH receptor modulator(s) may be administered to the patient at the same time as the CB1 antagonist(s) (e.g., as a single dosage unit), or may be administered separately (before or after CB1 antagonist). Within preferred embodiments, the MCH receptor modulator(s) and CB1 antagonist(s) are ultimately simultaneously present at effective concentrations in a body fluid (e.g., blood) of the patient. An effective concentration of MCH receptor modulator or CB1 antagonist is a concentration that is sufficient to reduce one or more of food consumption, appetite and/or body mass index in the patient when repeatedly coadministered as described herein.

Within separate aspects, the present invention provides a variety of in vitro uses for the compounds provided herein. For example, such compounds may be used as probes for the detection and localization of MCH receptors, in samples such as tissue sections, as positive controls in assays for receptor activity, as standards and reagents for determining the ability of a candidate agent to bind to MCH receptor, or as radiotracers for positron emission tomography (PET) imaging or for single photon emission computerized tomography (SPECT). Such assays can be used to characterize MCH receptors in living subjects. Compounds provided herein are also useful as standards and reagents in determining the ability of a test compound to bind to MCH receptor.

Within methods for determining the presence or absence of MCH receptor in a sample, a sample may be incubated with a compound as provided herein under conditions that permit binding of the compound to MCH receptor. The amount of compound bound to MCH receptor in the sample is then detected. For example, a compound may be labeled using any of a variety of well-known techniques (e.g., radiolabeled with a radionucleide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of MCH receptor in the sample. Detection assays, including receptor autoradiography (receptor mapping) of MCH receptors in cultured cells or tissue samples may be performed as described by Kuhar in sections 8.1.1 to 8.1.9 of Current Protocols in Pharmacology (1998) John Wiley & Sons, New York.

Compounds provided herein may also be used within a variety of well-known cell culture and cell separation methods. For example, compounds may be linked to the interior surface of a tissue culture plate or other cell culture support, for use in immobilizing MCH receptor-expressing cells for screens, assays and growth in culture. Compounds may also be used to facilitate cell identification and sorting in vitro, permitting the selection of cells expressing a MCH receptor. Preferably, the compound(s) for use in such methods are labeled as described herein. Within one preferred embodiment, a compound linked to a fluorescent marker, such as fluorescein, is contacted with the cells, which are then analyzed by fluorescence activated cell sorting (FACS).

Within other aspects, methods are provided for modulating binding of MCH to an MCH receptor in vitro or in vivo, comprising contacting a MCH receptor with a sufficient amount of a modulator provided herein, under conditions suitable for binding of MCH to the receptor. Preferably, within such methods, MCH binding to receptor is inhibited by the modulator. The MCH receptor may be present in solution, in a cultured or isolated cell preparation or within a patient. Preferably, the MCH receptor is a MCH1R receptor present in the hypothalamus. In general, the amount of compound contacted with the receptor should be sufficient to modulate MCH binding to MCH receptor in vitro within, for example, a binding assay as described in Example 33 and/or Example 36. MCH receptor preparations used to determine in vitro binding may be obtained from a variety of sources, such as from HEK 293 cells or Chinese Hamster Ovary (CHO) cells transfected with a MCH receptor expression vector, as described herein.

Also provided herein are methods for modulating the signal-transducing activity of cellular MCH receptors, by contacting MCH receptor, either in vitro or in vivo, with a sufficient amount of a modulator as described above, under conditions suitable for binding of MCH to the receptor. Preferably, within such methods, signal-transducing activity is inhibited by the modulator. The MCH receptor may be present in solution, in a cultured or isolated cell preparation or within a patient. In general, the amount of modulator contacted with the receptor should be sufficient to modulate MCH receptor signal transducing activity in vitro within, for example, a calcium mobilization assay as described in Example 37 and/or an agonist-stimulated GTP gamma³⁵S binding assay as described in Example 35. An effect on signal-transducing activity may be assessed as an alteration in the electrophysiology of the cells, using standard techniques, such as intracellular patch clamp recording or patch clamp recording. If the receptor is present in an animal, an alteration in the electrophysiology of the cell may be detected as a change in the animal's feeding behavior.

Preparation of MCH Receptor Modulators

Compounds provided herein may generally be prepared using standard synthetic methods. Starting materials are generally readily available from commercial sources, such as Sigma-Aldrich Corp. (St. Louis, Mo.). For example, a synthetic route similar to that shown in any one of the following Schemes may be used. It will be apparent that the final product and any intermediate(s) shown in the following schemes may be extracted, dried, filtered and/or concentrated, and may be further purified (e.g., by chromatography). Each variable (e.g., “R”) in the following Schemes, refers to any group consistent with the description of the compounds provided herein. An individual skilled in the art may find modifications of one or several of the synthetic steps described herein without diverting significantly from the overall synthetic scheme. Further experimental details for synthesis of representative compounds via these schemes are provided in Examples 1-30, herein.

In the following Schemes and elsewhere herein, the following abbreviations are used:
Ac acetyl
9-BBN 9-borabicyclo[3.3.1]nonane
BINAP [2,2′-bis(diphenylphosphino)-1,1′-binaphthyl]
BOP benzotriazol-1-yl-oxy-tris(dimethylamino)phosphonium hexafluorophosphate
DBU 1,8-diazabicyclo[5.4.0]undec-7-ene
DCC dicyclohexylcarbodiimide
DCM dichloromethane
DIPE diisopropyl ether
DMA dimethylamine
DMAP N,N-Dimethyl-4-aminopyridine
DMSO dimethyl sulfoxide
DMF dimethylformamide
DPPA diphenylphosphoryl azide
Et ethyl
EtOAc ethyl acetate
Et₂O diethyl ether
EtOH ethanol
Fe(acac)₃ Iron tris(acetylacetonate)
HOAc acetic acid
HMPA hexamethylphosphorotriamide
LDA lithium diisopropylamide
Me methyl
MeOH methanol
MTBE methyl t-butyl ether
NEt₃ triethylamine
NMO N-methylmorpholine N-oxide
OiPr isopropoxy
OTf trifluoromethanesulfonate
Pd₂(dba)₃ tris(dibenzylideneacetone)dipalladium (0)
PPh₃ triphenyl phosphine
pyBrop bromo-tris-pyrrolidine-phosphonium-hexafluorophosphate
PTLC preparative thin layer chromatography
TBDMS tert-butyl-dimethyl-silanyl
TFA trifluoroacetic acid
THF tetrahydrofuran
TLC thin layer chromatography
TPAP tetra-n-propylammonium perruthenate
h hour(s)
min minute(s)

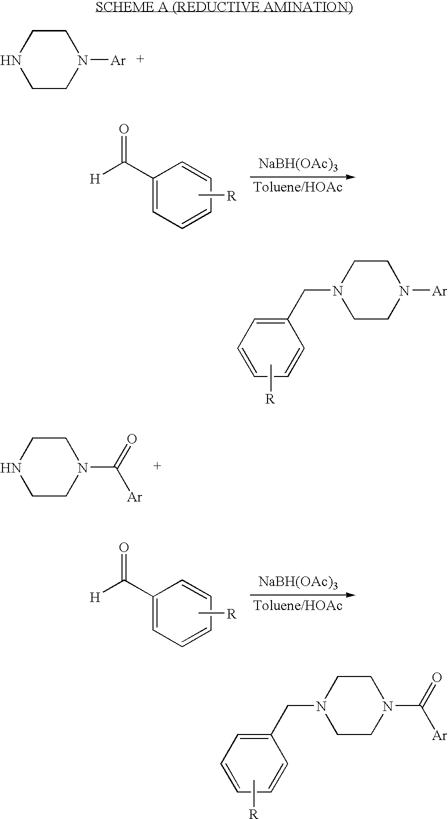

Briefly, one equivalent each of an appropriately substituted piperazine and an appropriately substituted benzaldehyde are reacted under acidic catalysis with an excess of NaBH(OAc)₃ under a nitrogen atmosphere until no starting material is detectable by TLC. At that time, the reaction is quenched with saturated aqueous NaHCO₃ and extracted with EtOAc to yield the appropriate 1-benzyl-4-substituted piperazine analogue. Extracts may be dried over anhydrous MgSO₄, concentrated in vacuo and chromatographed.

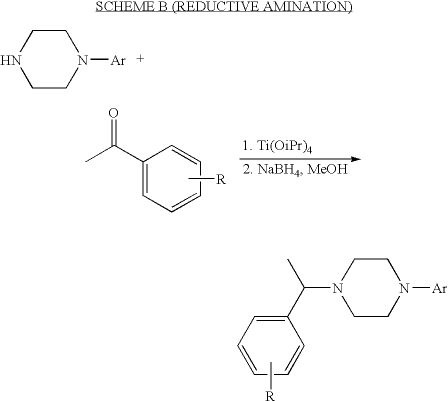

Briefly, one equivalent each of an appropriately substituted piperazine and an appropriately substituted acetophenone are heated with Ti(OiPr)₄ (e.g., 70° C. for 2 hours). The reaction solution is cooled and after dilution with MeOH, reacted with NaBH₄ to yield the 1-benzyl-4-aryl piperazine analogue. The reaction is quenched by the addition of 1 N NaOH and may be extracted with DCM. DCM extracts may be dried over anhydrous MgSO₄, concentrated in vacuo, and subjected to chromatography.

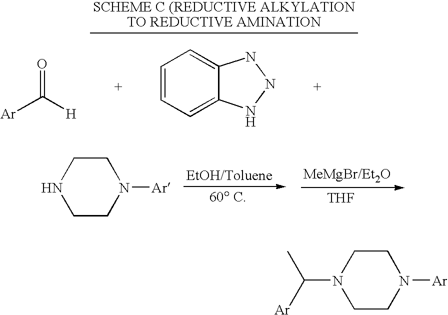

Briefly, a solution containing an appropriately substituted aromatic aldehyde, benzotriazole and an appropriately substituted aromatic piperazine in a mixture of EtOH and toluene is heated and the solution is concentrated. The residue is evaporated with toluene, then dissolved in THF and treated with an excess of methyl magnesium bromide in diethyl ether to yield the 1-benzyl-4-aryl piperazine analogue.

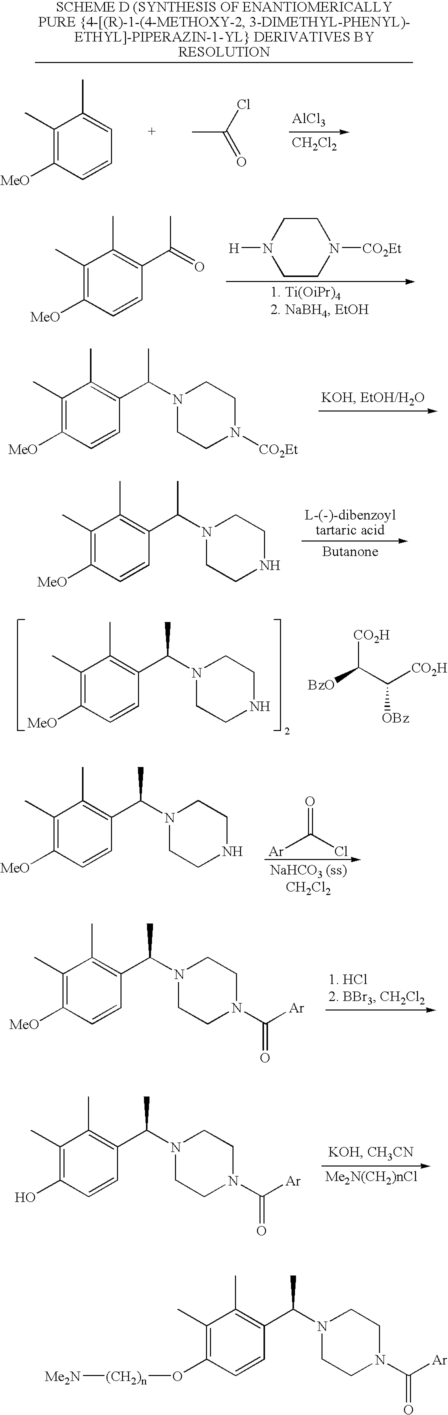

Briefly, 2,3-dimethylanisole is acylated by reaction with acetyl chloride and AlCl₃ under Friedel-Crafts reaction conditions to yield 1-(4-methoxy-2,3-dimethyl-phenyl)-ethanone. This is submitted to reductive amination reaction conditions (Scheme B) to produce racemic 4-[1-(4-methoxy-2,3-dimethylphenyl)-ethyl]-piperazine-1-carboxylic acid ethyl ester, which is converted to racemic 1-[1-(4-methoxy-2,3-dimethylphenyl)-ethyl]-piperazine by saponification with a strong base such as LiOH, NaOH, KOH and the like in the presence of a solvent mixture containing water and an alcohol such as MeOH, EtOH, isopropanol or n-butanol at temperatures between room temperature and the boiling point of the reaction mixture at atmospheric pressure. The racemic amine is resolved by salt formation (e.g., with L-(−)-dibenzoyltartaric acid in a solvent such as acetone, butanone, MeOH, EtOH, tetrahydrofuran, etc.). After converting the enantiomerically pure salt to its free base, acylation reaction with an appropriate acid chloride under Schotten-Baumann reaction conditions yields the corresponding 1-benzyl-4-aroyl piperazine analogue. Demethylation with a strong Lewis acid such as but not limited to BBr₃ yields the corresponding phenol, which is then alkylated with an appropriate electrophile to produce the final target compound

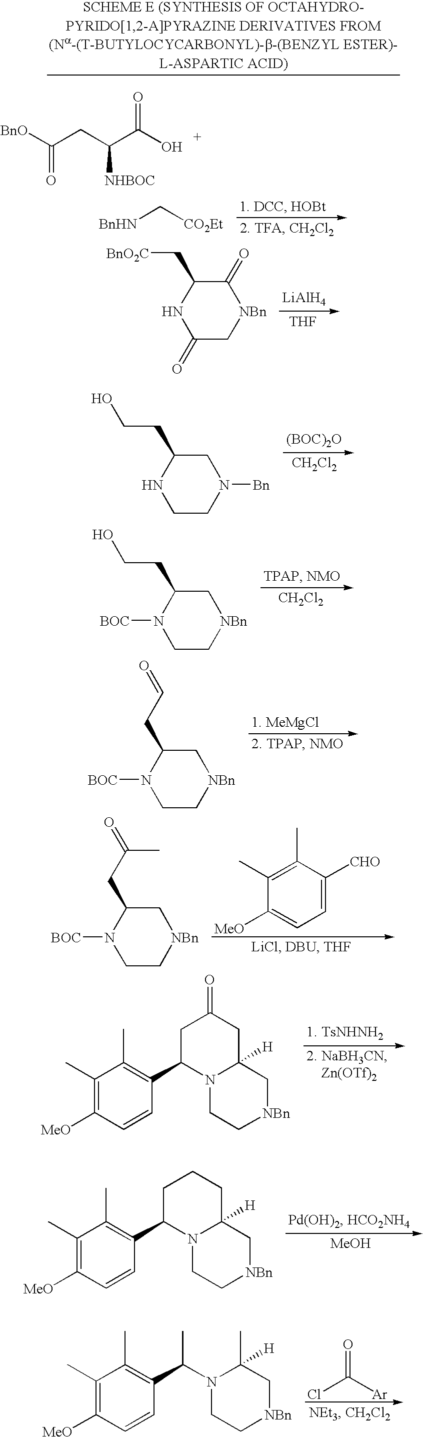

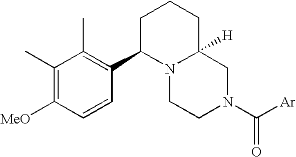

Briefly (essentially as described by WO 98/20001 and WO 99/65922), (N^{α}-(t-butyloxycarbonyl)-β-(benzyl ester)-L-aspartic acid) is reacted with N-benzylglycine in the presence of DCC and butanol to produce the corresponding N-benzylglycine amide, which is further reacted with TFA to remove the BOC protecting group, yielding ((S)-4-benzyl-3,6-dioxo-piperazin-2-yl)-acetic acid ethyl ester. This is reduced to 2-((S)-4-benzyl-piperazin-2-yl)-ethanol by reaction with LiAlH₄ in THF. As described by WO 02/094799, the free amine is reacted with (BOC)₂O to produce the corresponding carbamate, and the primary alcohol is oxidized with catalytic TPAP in the presence of NMO to the corresponding aldehyde, ((S)-4-benzyl-piperazin-2-yl)-acetaldehyde. This is reacted with MeMgCl under Grignard reaction conditions to produce the secondary alcohol, 1-((S)-4-benzyl-piperazin-2-yl)-propan-2-ol, as a mixture of diastereoisomers, which is oxidized to the corresponding methylketone, 1-((S)-4-benzyl-piperazin-2-yl)-propan-2-one, by reaction with catalytic TPAP and NMO. The methylketone undergoes a tandem aldol condensation/Michael conjugated addition by reaction with 1-(4-methoxy-2,3-dimethylphenyl)-ethanone in the presence of LiCl and DBU as a base in THF as the solvent, yielding bicyclic (6R,9aS)-2-benzyl-6-(4-methoxy-2,3-dimethyl-phenyl)-octahydro-pyrido[1,2-a]pyrazin-8-one. This is deoxygenated to (6R,9aS)-2-benzyl-6-(4-methoxy-2,3-dimethylphenyl)-octahydro-pyrido[1,2-a]pyrazine by conversion to the corresponding tosylhydrazone and subsequent reduction with NaBH₃CN in the presence of zinc triflate. The benzyl group is removed by catalytic transfer hydrogenation reaction promoted by Pd(OH)₂ in the presence of excess ammonium formate in MeOH. Finally, (6R,9aS)-6-(4-methoxy-2,3-dimethylphenyl)-octahydro-pyrido[1,2-a]pyrazine is converted to the desired heteroaroyl analog by reaction with the corresponding acid chloride under Schotten-Bauman reaction conditions.

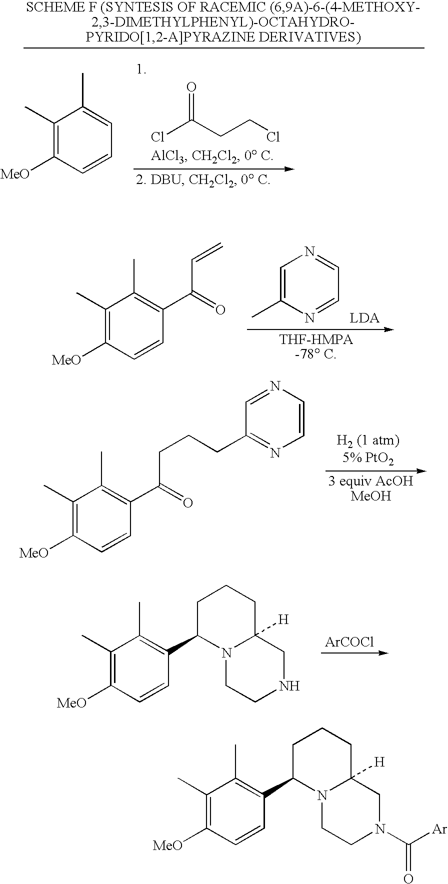

Briefly, 2,3-dimethylanisole is acylated with 3-choropropionyl chloride under Friedel-Crafts reaction conditions in the presence of AlCl₃ and the resulting 3-chloro-1-(4-methoxy-2,3-dimethylphenyl)-propan-1-one dehydrochlorinated by treatment with a base such as DBU in a solvent such as but not limited to DCM to produce the vinylic ketone 1-(4-methoxy-2,3-dimethyl-phenyl)-propenone. Michael addition of pyrazinylmethyllithium (obtained by reacting methylpyrazine with LDA in THF) yields 1-(4-methoxy-2,3-dimethyl-phenyl)-4-pyrazin-2-yl-butan-1-one. Transformation to (6,9a)-6-(4-methoxy-2,3-dimethylphenyl)-octahydro-pyrido[1,2-a]pyrazine is accomplished by a one-pot sequence involving catalytic hydrogenation with H₂ at atmospheric pressure in the presence of catalytic amounts of Adams catalyst and acetic acid in MeOH as the solvent. Finally, the desired heteroaryl analogue, [(6,9a)-6-(4-methoxy-2,3-dimethylphenyl)-octahydro-pyrido[1,2-a]pyrazin-2-yl]-heteroaryl-methanone, is obtained by reaction with the corresponding acid chloride under Schotten-Baumann reaction conditions.

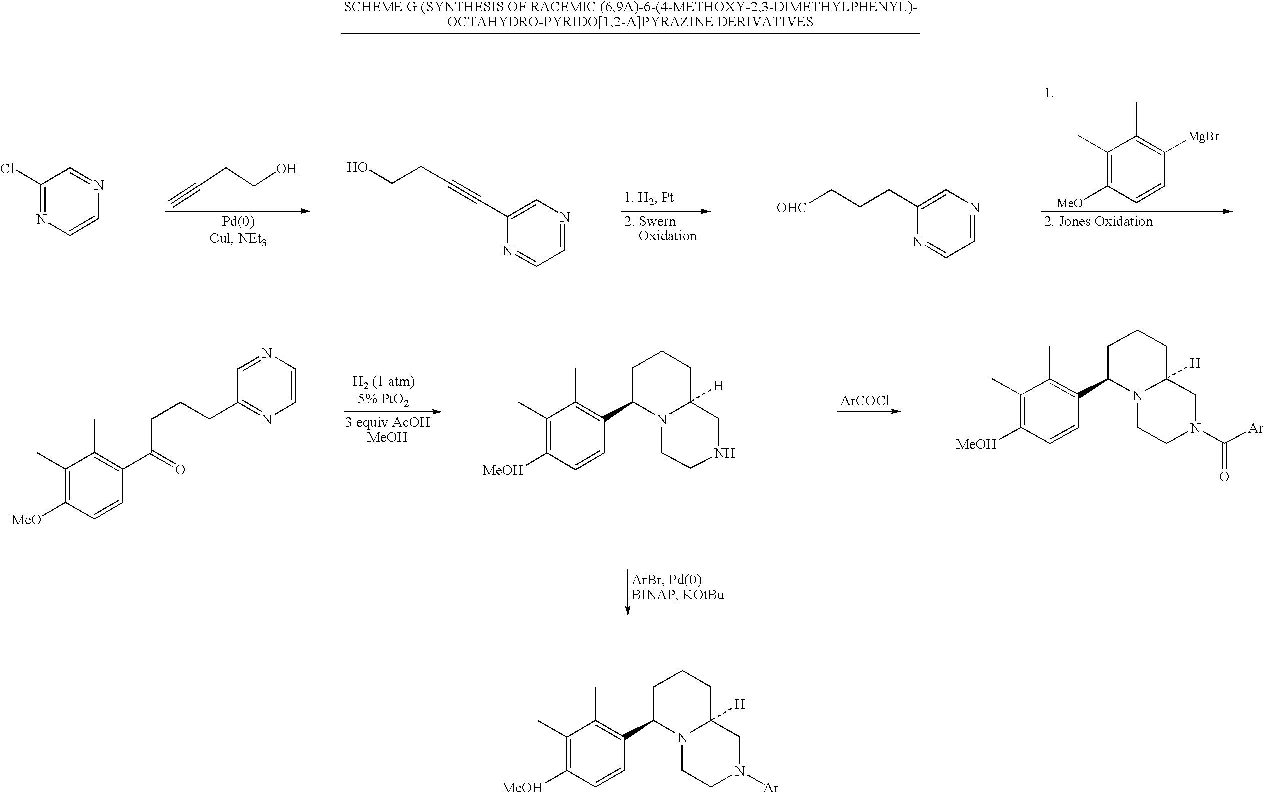

Briefly, 2-chloropyrazine is transformed into 4-pyrazin-2-yl-but-3-yn-1-ol by Pd-catalyzed reaction with 3-butyn-1-ol in the presence of CuI as cocatalyst and a base such as but not limited to NEt₃, piperidine, N-methylmorpholine and the like. The alkyne is reduced by catalytic hydrogenation in the presence of Pd/C to 4-pyrazin-2-yl-butan-1-ol. The alcohol is oxidized to the corresponding aldehyde, 4-pyrazin-2-yl-butyraldehyde. Grignard reaction with 2,3-dimethyl-4-methoxyphenylmagnesium bromide under anhydrous conditions in a solvent such as but not limited to Et₂O, THF, DIPE, MTBE or dibutyl ether affords 1-(4-methoxy-2,3-dimethyl-phenyl)-4-pyrazin-2-yl-butan-1-ol. Jones oxidation yields 1-(4-methoxy-2,3-dimethyl-phenyl)-4-pyrazin-2-yl-butan-1-one. Transformation to (6,9a)-6-(4-methoxy-2,3-dimethylphenyl)-octahydro-pyrido[1,2-a]pyrazine is accomplished by a one-pot sequence involving catalytic hydrogenation with H₂ at atmospheric pressure in the presence of catalytic amounts of Adams catalyst and acetic acid in MeOH as the solvent. Finally, the desired heteroaryl analogue, [(6,9a)-6-(4-methoxy-2,3-dimethylphenyl)-octahydro-pyrido[1,2-a]pyrazin-2-yl]-heteroaryl-methanone, is obtained by reaction with the corresponding acid chloride under Schotten-Baumann reaction conditions. Alternatively, (6,9a)-6-(4-methoxy-2,3-dimethylphenyl)-octahydro-pyrido[1,2-a]pyrazine can be reacted with an aryl halide, triflate or tosylate under Pd(0) catalysis to produce the corresponding (6,9a)-6-(4-methoxy-2,3-dimethyl-phenyl)-2-aryl-octahydro-pyrido[1,2-a]pyrazine.

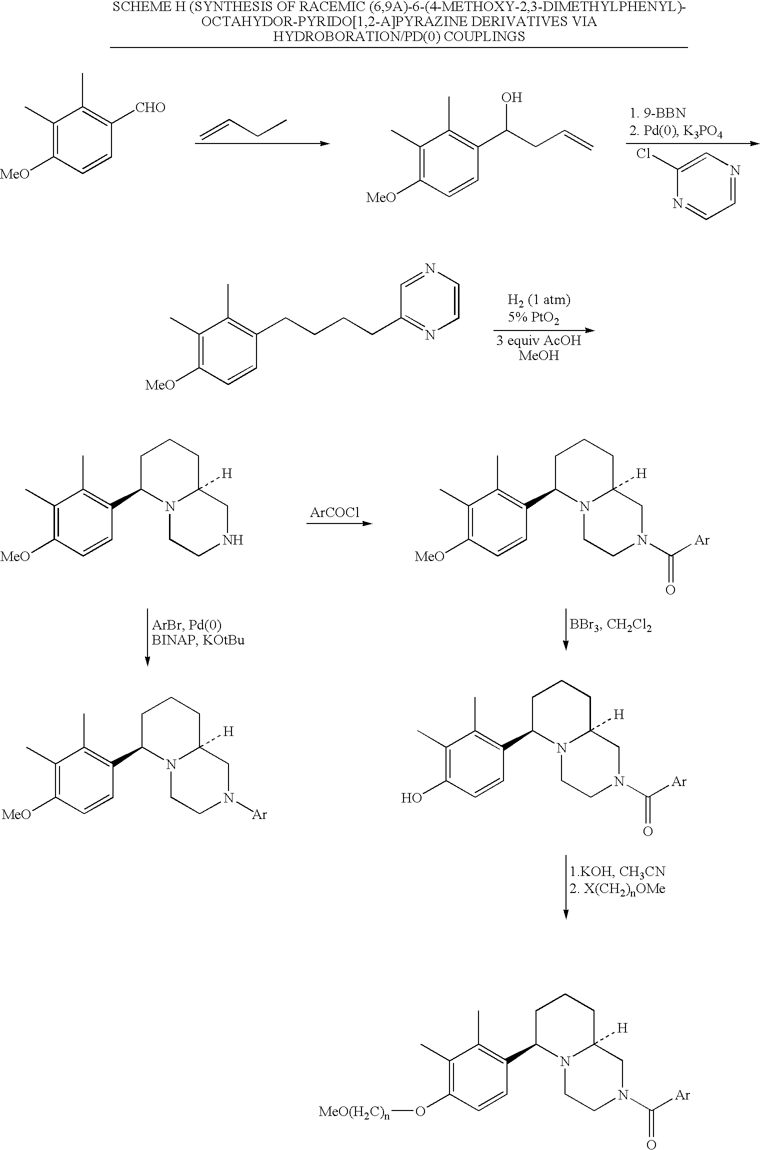

Briefly, 2,3-dimethyl-4-methoxybenzaldehyde is reacted under Grignard reaction conditions with allylmagnesium bromide in a solvent such as but not limited to THF, Et₂O or MTBE, at temperatures between −78° C. and 20° C. to produce the corresponding alcohol, 1-(4-methoxy-2,3-dimethylphenyl)but-3-en-1-ol. This is submitted to a hydroboration reaction with 9-BBN (or similar hydroborating reagent), followed by Pd(0)-catalyzed coupling reaction with 2-chloropyrazine in a solvent like THF and similar to yield 1-(4-methoxy-2,3-dimethylphenyl)-4-pyrazin-2-yl-butan-1-ol. This alcohol is oxidized to the corresponding ketone with, for example, CrO₃ in H₂SO₄/acetone (Jones reagent), N-methylmorpholine N-oxide in the presence of catalytic amounts of TPAP and 4 Angstrom molecular sieves in a solvent such as dry DCM, or Dess-Martin reagent. 1-(4-Methoxy-2,3-dimethyl-phenyl)-4-pyrazin-2-yl-butan-1-one is transformed to (6,9a)-6-(4-methoxy-2,3-dimethylphenyl)-octahydro-pyrido[1,2-a]pyrazine by a one-pot sequence involving catalytic hydrogenation with H₂ at atmospheric pressure in the presence of catalytic amounts of Adams catalyst and acetic acid in MeOH as the solvent. [(6,9a)-6-(4-Methoxy-2,3-dimethylphenyl)-octahydro-pyrido[1,2-a]pyrazin-2-yl]-heteroaryl-methanone is obtained by reaction with the corresponding acid chloride under Schotten-Baumann reaction conditions. Demethylation with a strong Lewis acid such as, but not limited to, BBr₃ yields the corresponding phenol, which is then alkylated with an appropriate electrophile to produce the final target compound. Alternatively, (6,9a)-6-(4-methoxy-2,3-dimethylphenyl)-octahydro-pyrido[1,2-a]pyrazine can be reacted with an aryl halide, triflate or tosylate under Pd(0) catalysis to produce the corresponding (6,9a)-6-(4-methoxy-2,3-dimethyl-phenyl)-2-aryl-octahydro-pyrido[1,2-a]pyrazine.

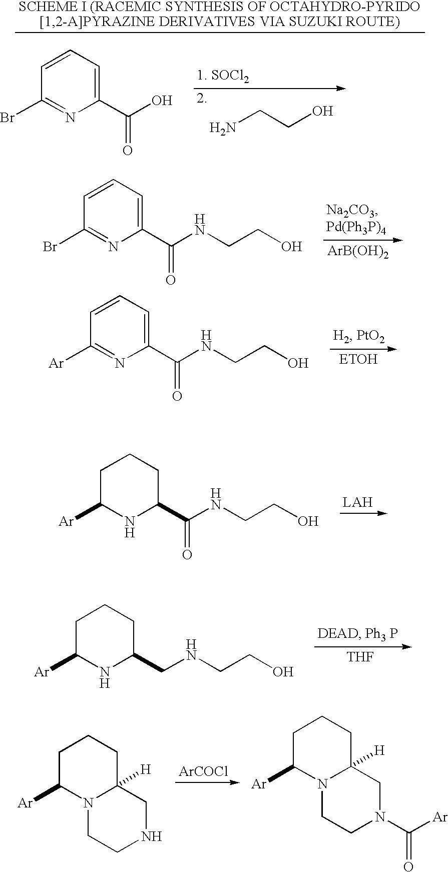

Briefly, 5-bromopicolinic acid is reacted with thionyl chloride, followed by ethanolamine to yield the corresponding amide, 6-bromopyridine-2-carboxylic acid (2-hydroxyethyl)-amide. The amide is then reacted under Suzuki reaction conditions with an aryl boronic acid, KOtBu and catalytic Pd₂(dba)₃ until TLC shows no detectable starting material to produce the 6-aryl-pyridine-2-carboxylic acid (2-hydroxy-ethyl)-amide. Reduction of the pyridine ring to the 2,6-cis disubstituted piperidine compound, followed by LiAlH₄ reduction of the amide group yields the aminoalcohol 2-[(6-aryl-pyridin-2-ylmethyl)-amino]-ethanol. Intramolecular Mitsunobu reaction is achieved using PPh₃ and diethyl azodicarboxylate, to yield (6,9a)-6-(4-methoxy-2,3-dimethylphenyl)-octahydro-pyrido[1,2-a]pyrazine. Finally, (6,9a)-6-(4-methoxy-2,3-dimethylphenyl)-octahydro-pyrido[1,2-a]pyrazine is converted to the desired heteroaroyl analog by reaction with the corresponding acid chloride under Schotten-Baumann reaction conditions.

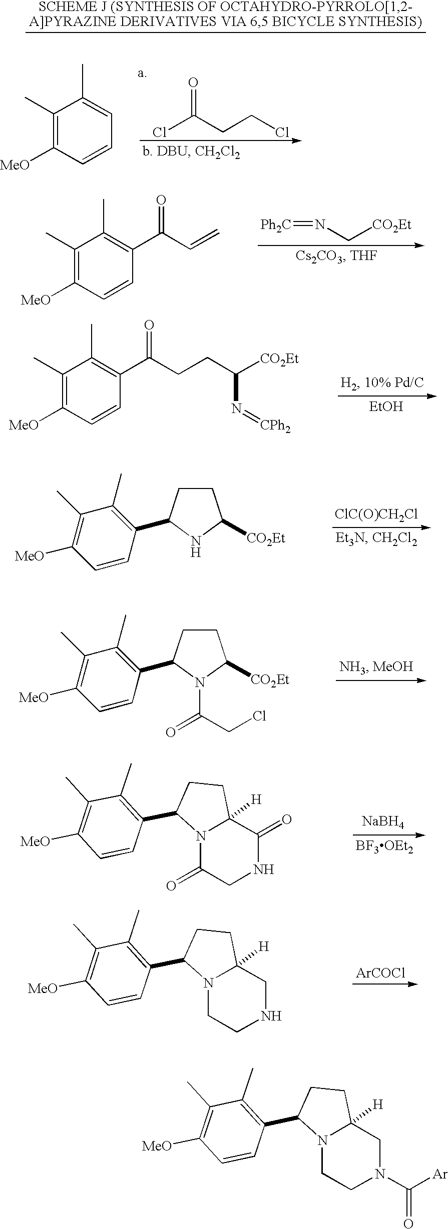

Briefly, 2,3-dimethylanisole is acylated with 3-choropropionyl chloride under Friedel-Crafts reaction conditions in the presence of AlCl₃ and the resulting 3-chloro-1-(4-methoxy-2,3-dimethylphenyl)-propan-1-one dehydrochlorinated by treatment with a base such as DBU in a solvent such as but not limited to DCM to produce the vinylic ketone 1-(4-methoxy-2,3-dimethylphenyl)-propenone. Michael addition of (benzhydrylidene-amino)-acetic acid ethyl ester in the presence of Cs₂CO₃ as a base yields 2-(benzhydrylidene-amino)-5-(4-methoxy-2,3-dimethyl-phenyl)-5-oxo-pentanoic acid ethyl ester. Upon hydrogenolysis with H₂ in the presence of catalytic Pd10%/C in EtOH as the solvent, this cyclizes to 2,5-cis-5-(4-methoxy-2,3-dimethyl-phenyl)-pyrrolidine-2-carboxylic acid ethyl ester, which reacts with chloroacetyl chloride in the presence of NEt₃ in a solvent such as but not limited to DCM to furnish 2,5-cis-1-(2-chloro-acetyl)-5-(4-methoxy-2,3-dimethyl-phenyl)-pyrrolidine-2-carboxylic acid ethyl ester. Upon treatment with ammonia in alcohol the chloroamide cyclizes to the corresponding cis-(6,8a)-6-(4-methoxy-2,3-dimethylphenyl)-hexahydro-pyrrolo[1,2-a]pyrazine-1,4-dione, which is reduced to cis-(6,8a)-6-(4-methoxy-2,3-dimethylphenyl)-octahydro-pyrrolo[1,2-a]pyrazine by treatment with NaBH₄ in the presence of BF₃.OEt₂. Finally, the desired heteroaryl analogue, cis-[(6,8a)-6-(4-methoxy-2,3-dimethylphenyl)-hexahydro-pyrrolo[1,2-a]pyrazin-2-yl]-aryl-methanone is obtained by reaction with the corresponding acid chloride under Schotten-Baumann reaction conditions. Demethylation with a strong Lewis acid such as, but not limited to, BBr₃ yields the corresponding phenol, which is then alkylated with an appropriate electrophile to produce the final target compound.

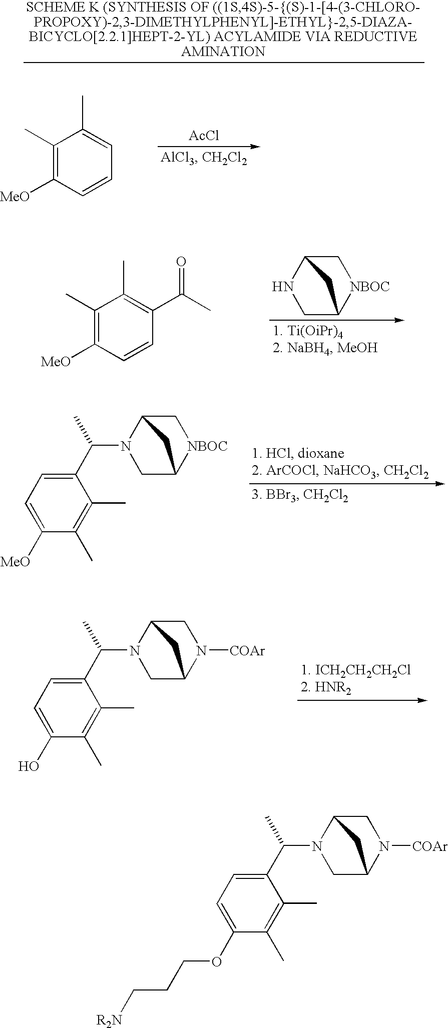

Briefly, 2,3-dimethylanisole is acylated with acetyl chloride under Friedel-Crafts reaction conditions in the presence of AlCl₃ and the resulting acetophenone, 1-(4-methoxy-2,3-dimethyl-phenyl)-ethanone. Reductive amination with (1S,4S)-2,5-diaza-bicyclo[2.2.1]heptane-2-carboxylic acid tert-butyl ester under the reaction conditions of Scheme B [Ti(OiPr)₄, NaBH₄, MeOH) yields (1S,4S)-5-[1-(4-methoxy-2,3-dimethylphenyl)-ethyl]-2,5-diaza-bicyclo[2.2.1]-heptane-2-carboxylic acid tert-butyl ester as a mixture of C-5 diastereoisomers (Ratio 5-S/5-R=2:1). The desired diastereoisomer, (1S,4S)-5-[(S)-1-(4-methoxy-2,3-dimethylphenyl)-ethyl]-2,5-diaza-bicyclo[2.2.1]heptane-2-carboxylic acid tert-butyl ester is separated by flash chromatography. The BOC protecting group is removed, for example, by treatment with HCl in dioxane or similar reagent(s) and (1S,4S)-2-[(S)-1-(4-methoxy-2,3-dimethylphenyl)-ethyl]-2,5-diaza-bicyclo[2.2.1]-heptane is acylated with an acid chloride ArCOCl under-reaction conditions to furnish the corresponding {(1S,4S)-5-[(S)-1-(4-methoxy-2,3-dimethylphenyl)-ethyl]-2,5-diaza-bicyclo[2.2.1]-hept-2-yl}-acylamide. Treatment of the HCl salt of this amide with BBr₃ in solvents such as dichloromethane yields the phenol, {(1S,4S)-5-[(S)-1-(4-hydroxy-2,3-dimethylphenyl)-ethyl]-2,5-diaza-bicyclo[2.2.1]hept-2-yl}-acylamide. This is alkylated, for example, with 1-chloro-3-iodopropane in a solvent such as acetonitrile, acetone or the like in the presence of a promoter such as KOH, Cs₂CO₃, K₃PO₄ or similar base(s) to produce ((1S,4S)-5-{(S)-1-[4-(3-chloro-propoxy)-2,3-dimethylphenyl]-ethyl}-2,5-diaza-bicyclo-[2.2.1]hept-2-yl) acylamide. Reaction with a nucleophile such as an amine, alcohol, thiol or heterocycle in the presence of a base such as K₂CO₃ and in a solvent such as acetonitrile, propionitrile, acetone, DMF or DMSO yields the final target compound.

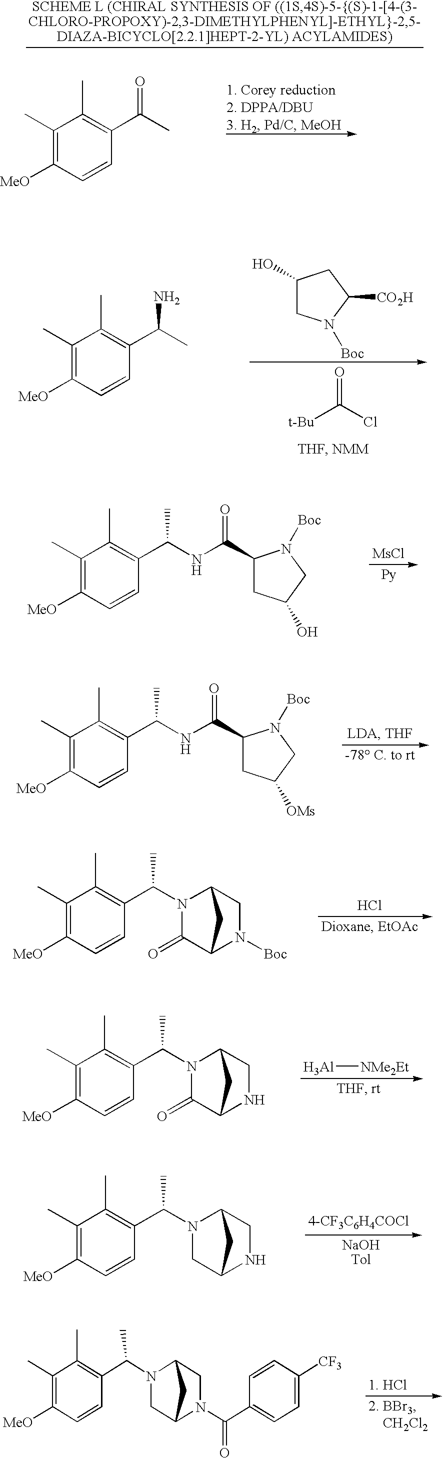

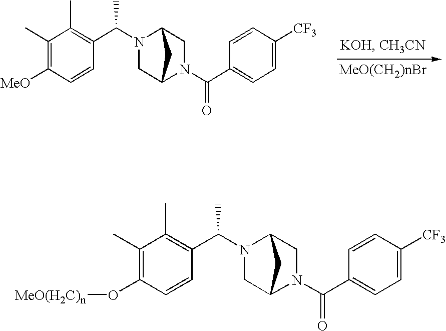

Briefly, 1-(4-methoxy-2,3-dimethylphenyl)-ethanone is converted to the corresponding chiral alcohol (S)-1-(4-methoxy-2,3-dimethyl-phenyl)-ethanol by reaction with catalytic amounts of (S)-2-methyl-CBS-oxazaborolidine (Aldrich Chemical Co.) in the presence of BH₃.SMe₂ as the reducing agent. The chiral alcohol is converted to 1-((S)-1-azido-ethyl)-4-methoxy-2,3-dimethylbenzene by reaction with DPPA and DBU. The azide is reduced to the chiral amine (S)-1-(4-methoxy-2,3-dimethyl-phenyl)-ethylamine by catalytic hydrogenation in the presence of Pd/C and MeOH as the reaction solvent. This amine is converted to the corresponding amide by reaction with (2S,4R)-4-hydroxy-pyrrolidine-1,2-dicarboxylic acid 1-tert-butyl ester in the presence of pivaloyl chloride and N-methylmorpholine as a proton scavenger. Reaction with mesyl chloride affords (2S,4R)-4-methanesulfonyloxy-2-[(S)-1-(4-methoxy-2,3-dimethyl-phenyl)-ethylcarbamoyl]-pyrrolidine-1-carboxylic acid tert-butyl ester, which is submitted to intramolecular alkylation by treatment with LDA in THF at −78° C. to afford the lactam (1S,5S)-5-[(S)-1-(4-methoxy-2,3-dimethylphenyl)-ethyl]-6-oxo-2,5-diaza-bicyclo[2.2.1]heptane-2-carboxylic acid tert-butyl ester. The N-BOC group is removed by treatment with HCl, and the resulting aminolactam is reduced with alane-dimethylethylamine complex to the corresponding piperazine, (2S,4S)-2-[(S)-1-(4-methoxy-2,3-dimethylphenyl)-ethyl]-2,5-diaza-bicyclo[2.2.1]-heptane. {(1S,5S)-5-[(S)-1-(4-methoxy-2,3-dimethyl-phenyl)-ethyl]-2,5-diaza-bicyclo[2.2.1]-hept-2-yl}-(4-trifluoromethyl)-benzamide is obtained by reaction with the corresponding acid chloride under Schotten-Baumann reaction conditions. Demethylation with a strong Lewis acid such as, but not limited to, BBr₃ yields the corresponding phenol, which is then alkylated with an appropriate electrophile to produce the final target compound.

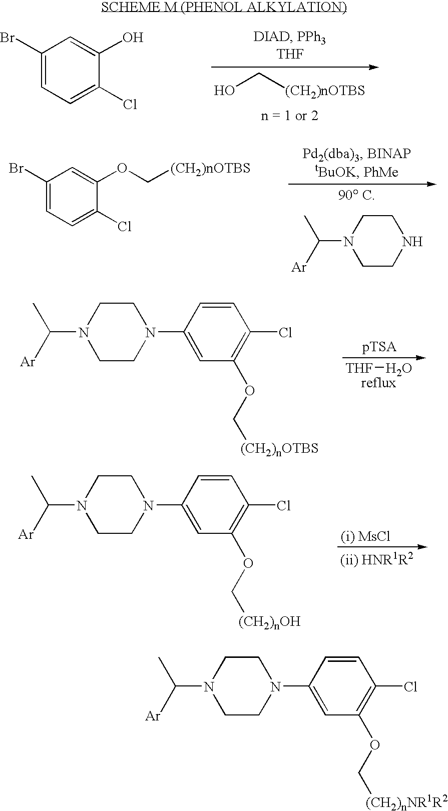

Briefly, 5-bromo-2-chlorophenol is alkylated following the Mitsunobu protocol by reaction with a monoprotected diol (for example, the mono-TBS ether of propylenglycol) in the presence of PPh₃ and diisopropyl azodicarboxylate and in THF as the reaction solvent. The resulting bromide is submitted to a Pd-catalyzed amine arylation reaction by reaction with 1-[1-(3,4-dimethoxyphenyl)-ethyl]-piperazine in the presence of potassium tert-butoxide as the base and catalytic amounts of BINAP and Pd₂(dba)₃ at temperatures around 90° C. The corresponding arylpiperazine is converted to the free alcohol by deprotecting the TBS group by treatment with an acidic catalyst such as p-toluenesulfonic acid at reflux temperature in a solvent mixture composed of water and THF. The primary alcohol is converted to the desired amine by first transforming it into the mesylate (MsCl, NEt₃) followed by reaction with excess amine.

In certain situations, compounds of the present invention may contain one or more asymmetric carbon atoms, so that the compounds can exist in different stereoisomeric forms. These compounds can be, for example, racemates or optically active forms. As noted above, all stereoisomers are encompassed by the present invention. Nonetheless, it may be desirable to obtain single enantiomers (i.e., optically active forms). Standard methods for preparing single enantiomers include asymmetric synthesis and resolution of the racemates. Resolution of the racemates can be accomplished, for example, by conventional methods such as crystallization in the presence of a resolving agent, or chromatography using, for example, a chiral HPLC column. As noted above, for compounds having an alpha-methyl benzyl group (R₃ is methyl, R₄ is hydrogen) the R enantiomer is generally preferred. Asymmetric synthesis of such compounds may be performed using the methods illustrated in Scheme D.

Compounds may be labeled by carrying out their synthesis using precursors comprising at least one atom that is an isotope. Each isotope is preferably carbon (e.g., ¹⁴C), hydrogen (e.g., ³H or 2H), fluorine (e.g., ¹⁸F), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or exchange with tritium gas under heterogeneous catalysis using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

The following Examples are offered by way of illustration and not by way of limitation. Unless otherwise specified all reagents and solvent are of standard commercial grade and are used without further purification.

### EXAMPLES

Mass spectra (MS) reported in the following Examples are collected using electrospray MS, obtained in positive ion mode using a Waters ZMD II Mass Spectrometer. MS conditions are as follows:
Capillary voltage: 3.5 kV
Cone voltage: 30 V
Desolvation and source temperature: 250° C. and 120° C. respectively
Mass range: 100-750
Scan time: 0.5 second
Inter scan delay: 0.1 minute

### Example 1

### {(6R,9AS)-6-[4-(2-METHOXY-ETHOXY)-2,3-DIMETHYLPHENYL]-OCTAHYDRO-PYRIDO[1,2-A]PYRAZIN-2-YL}-(6-TRIFLUOROMETHYL-PYRIDIN-3-YL)-METHANONE

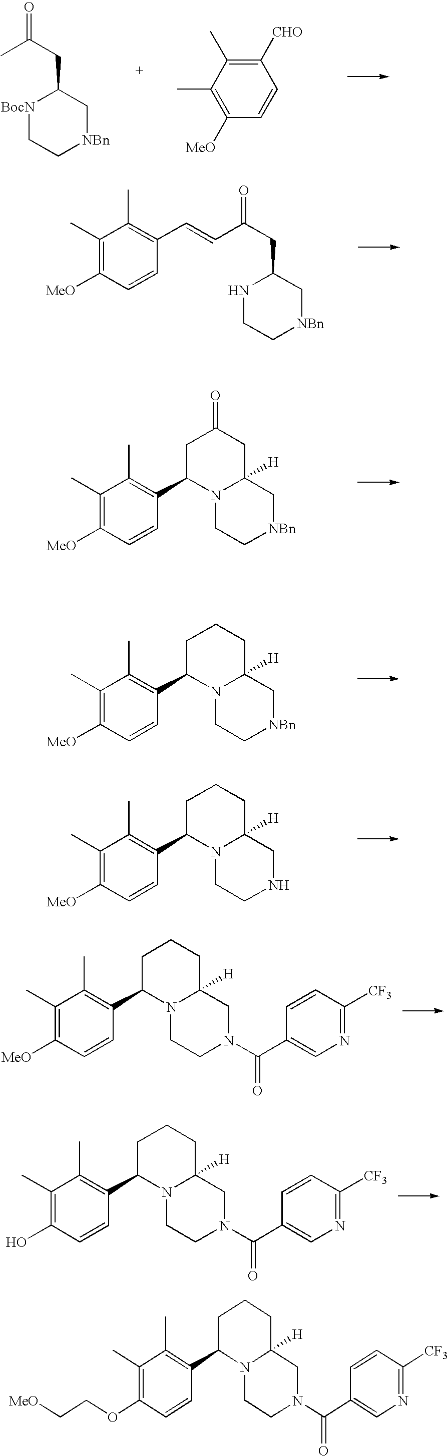

### Step 1. (E)-1-((S)-4-Benzyl-piperazin-2-yl)-4-(4-methoxy-2,3-dimethylphenyl)-but-3-en-2-one

(S)-4-Benzyl-2-(2-oxopropyl)-piperazine-1-carboxylic acid tert-butyl ester (15.0 g, 45.0 mmol, obtained as in WO 02/094799), 2,3-dimethylanisaldehyde (8.9 g, 54.0 mmol, 1.2 eq), and lithium chloride (9.6 g, 226.0 mmol, 5.0 eq) are stirred together in 225 mL of anhydrous THF under a nitrogen atmosphere for 40 min at ambient temperature to effect dissolution of the lithium chloride. This solution is cooled to 0° C. and treated with DBU (7.45 mL, 49.8 mmol, 1.1 eq.), which is added slowly, dropwise via syringe. The mixture is stirred and allowed to slowly warm to ambient temperature. After 22 h, the mixture is diluted with H₂O (200 mL) and extracted with EtOAc (3×200 mL). The combined organic extracts are dried over Na₂SO₄, filtered and concentrated in vacuo to provide 23.0 g of the BOC-protected enone as a mixture of cis- and trans-isomers, which is used without additional purification. LC/MS: 479 (M+1). This material is dissolved in MeOH (225 mL) and diluted HCl (6N, 52.5 mL) and heated in a 60° C. oil bath for 3 h. After cooling, the solution is concentrated in vacuo. The residue is suspended in MeOH (150 mL) and reconcentrated in vacuo. This step is repeated four times to complete water removal, leaving the desired crude enone as a red solid, which is used without purification. LC/MS: 379 (M+1).

### Step 2. (6R,9aS)-2-Benzyl-6-(4-methoxy-2,3-dimethylphenyl)-octahydro-pyrido[1,2-a]pyrazin-8-one

The crude enone from step 1 is dissolved in 300 mL MeOH and treated with 160 mL of 2M ammonium acetate. The mixture is stirred at ambient temperature for 14.5 h, then at 60° C. for 2 h. The MeOH is removed in vacuo and the aqueous residue extracted with DCM (3×250 mL). The combined extracts are dried over Na₂SO₄, filtered and concentrated. The residue is purified by flash chromatography on silica gel using 80-60% hexanes/EtOAc as eluent to afford (6R,9aS)-2-benzyl-6-(4-methoxy-2,3-dimethylphenyl)-octahydro-pyrido[1,2-a]pyrazin-8-one as a white foam. LC/MS: 379 (M+1). ¹H NMR (400 MHz, CDCl₃): 7.79 (6H, m), 6.73 (1H, m), 3.80 (3H, s), 3.70 (1H, bs), 3.50 (2H, dd), 3.19 (1H, m), 2.79-2.28 (7H, bm), 2.25-1.94 (9H, bm).

### Step 3. (6R,9aS)-2-Benzyl-6-(4-methoxy-2,3-dimethylphenyl)-octahydro-pyrido[1,2-a]pyrazine

(6R,9aS)-2-Benzyl-6-(4-methoxy-2,3-dimethylphenyl)-octahydro-pyrido[1,2-a]pyrazin-8-one (9.48 g, 25.0 mmol) is stirred with p-toluenesulfonyl hydrazide (5.60 g, 30.0 mmol, 1.2 eq) in 40 mL anhydrous THF and 200 mL anhydrous MeOH for 20 h at ambient temperature under a nitrogen atmosphere. LC/MS analysis indicates complete conversion to the p-toluenesulfonyl hydrazone. The solution is sparged with argon for 30 min and then treated with 50 mL of a 1.5 M solution of NaCNBH₃ in MeOH. Zinc trifluoromethanesulfonate (140 mg, 0.376 mmoles, 1.5%) is added and the solution is heated in a 65° C. oil bath for 5.5 h under an argon balloon. LC/MS-analysis indicates consumption of the hydrazone. The mixture is allowed to cool and is quenched with 500 mL of saturated NaHCO₃. After stirring vigorously for 30 min, the mixture is extracted with DCM (4×200 mL). The combined extracts are dried over Na₂SO₄, filtered and concentrated in vacuo. The residue is purified by column chromatography on silica gel eluting with 90-80% hexanes/EtOAc to yield (6R,9aS)-2-benzyl-6-(4-methoxy-2,3-dimethylphenyl)-octahydro-pyrido[1,2-a]pyrazine as an oil. LC/MS: 365 (M+1). ¹H NMR (400 MHz, CDCl₃): 7.34 (1H, d), 7.27 (5H, m), 6.72 (1H, d), 3.79 (3H, s), 3.47 (2H, dd), 3.28 (1H, d), 2.68 (3H, m), 2.28-2.04 (8H, bm), 1.98-1.88 (2H, bm), 1.75 (1H, m), 1.59 (1H, d), 1.50-1.30 (4H, bm).

### Step 4. (6R,9aS)-6-(4-Methoxy-2,3-dimethylphenyl)-octahydro-pyrido[1,2-a]pyrazine

A solution containing the compound obtained in step 3 (2.66 g, 7.30 mmol) and ammonium formate (6.90 g, 109.50 mmol, 15 eq) is treated with 665 mg of 20% palladium hydroxide on carbon, and heated at reflux under a nitrogen balloon for 2 h. The mixture is filtered through a celite pad. The pad is washed with 200 mL of chloroform and the solution is concentrated in vacuo. The residue is taken up in 200 mL dichloromethane and washed with 1N NaOH, water, and brine (75 mL each) to remove any residual ammonium formate. The organic solution is concentrated in vacuo to afford (6R,9aS)-6-(4-methoxy-2,3-dimethylphenyl)-octahydro-pyrido[1,2-a]pyrazine as an amber colored oil which is used in the next step with no further purification. LC/MS: 275 (M+1). ¹H NMR (400 MHz, CDCl₃): 7.36 (1H, dd), 6.74 (1H, dd), 4.50 (1H, dd), 3.80 (3H, s), 3.28 (1H, d), 2.86 (1H, dd), 2.78 (2H, m), 2.67-2.55 (3H, m), 2.22 (3H, s), 2.17 (3H, s), 1.78-1.67 (4H, bm), 1.56-1.31 (4H, bm).

### Step 5. [(6R,9aS)-6-(4-Methoxy-2,3-dimethylphenyl)-octahydro-pyrido[1,2-a]pyrazin-2-yl]-(6-trifluoromethyl-pyridin-3-yl)-methanone

A magnetically stirred suspension of 6-trifluoromethylnicotinic acid (1.54 g, 8.07 mmol) in 50 mL of anhydrous DCM (0.16M), under nitrogen, is treated with oxalyl chloride (2M in DCM, 10.0 mL, 20.0 mmol, 2.5 eq) followed by the careful dropwise addition of 250 μL of DMF. Vigorous gas evolution ensues and the mixture becomes homogeneous. The solution is stirred at ambient temperature for 1.5 h, and then concentrated in vacuo to produce the acid chloride as a white solid. This solid is suspended in toluene and concentrated again and used with no further purification.

A solution of (6R,9aS)-6-(4-methoxy-2,3-dimethylphenyl)-octahydro-pyrido[1,2-a]pyrazine (1.77 g, 6.45 mmol) in anhydrous DCM (50 mL) is treated with NEt₃ (1.4 mL, 10.08 mmol) and DMAP (78.8 mg, 0.65 mmol). This mixture is stirred under nitrogen and treated with a solution of the previously prepared acid chloride in 10 mL DCM (an additional 5 mL is used as a rinse). The mixture is stirred at ambient temperature for 18 h and quenched by the addition of 80 mL 50% saturated NaHCO₃. The phases are separated and the aqueous phase is extracted twice with DCM. The combined extracts are dried over Na₂SO₄, filtered, and concentrated in vacuo. The residue is purified using flash chromatography on silica gel eluting with 70%-60% hexanes/EtOAc to give [(6R,9aS)-6-(4-methoxy-2,3-dimethylphenyl)-octahydro-pyrido[1,2-a]pyrazin-2-yl]-(6-trifluoromethyl-pyridin-3-yl)-methanone as a white foam. LC/MS: 448 (M+1). ¹H NMR (mixture of rotamers, 400 MHz, CDCl₃): 8.74 (1H, d), 7.90 (1H, dd), 7.76 (1H, dd), 7.34 (1H, dd), 6.74 (1H, dd), 4.50 (1H, dd), 3.79 (3H, d), 3.42-3.32 (2H, bm), 3.23-3.00 (1H, m), 2.91-2.53 (3H, bm), 2.21-2.14 (6H, m), 1.90-1.74 (4H, bm), 1.52-1.30 (3H, bm).

### Step 6. [(6R,9aS)-6-(4-Hydroxy-2,3-dimethylphenyl)-octahydro-pyrido[1,2-a]pyrazin-2-yl]-(6-trifluoromethyl-pyridin-3-yl)-methanone

A DCM solution of [(6R,9aS)-6-(4-methoxy-2,3-dimethylphenyl)-octahydro-pyrido[1,2-a]pyrazin-2-yl]-(6-trifluoromethyl-pyridin-3-yl)-methanone obtained in step 5 (2.30 g, 5.14 mmol) is treated with 15.4 mL of HCl (1 M in diethyl ether) and allowed to stand for 10 min. This solution is concentrated in vacuo and then dissolved in 70 mL anhydrous DCM. The resulting solution is cooled to −70° C. (dry ice/isopropanol bath) under nitrogen and treated with BBr₃ (1 M in DCM, 20.6 mL) dropwise via syringe over 20 min. The mixture is stirred for 18 h while warming to ambient temperature. After this time, the mixture is cooled to 0° C., treated with 150 mL saturated NaHCO₃ and stirred vigorously for 30 min. The phases are separated and the aqueous phase is extracted three times with DCM. The combined extracts are dried over Na₂SO₄, filtered and concentrated in vacuo to afford [(6R,9aS)-6-(4-hydroxy-2,3-dimethylphenyl)-octahydro-pyrido[1,2-a]pyrazin-2-yl]-(6-trifluoromethyl-pyridin-3-yl)-methanone as a light brown solid which is used without additional purification. LC/MS: 434 (M+1). ¹H NMR (mixture of rotamers, 400 MHz, CDCl₃): 8.74 (1H, d), 7.94 (1H, dd), 7.88 (1H, dd), 7.22 (1H, dd), 6.64 (1H, dd), 4.92 (1H, bs), 4.50 (1H, dd), 3.41-3.30 (2H, bm), 3.21 (1H, m), 3.03 (1H, m), 2.91-2.53 (3H, bm), 2.25-2.14 (6H, m), 1.92-1.58 (5H, bm), 1.20-1.32 (3H, bm).

### Step 7. {(6R,9aS)-6-[4-(2-Methoxy-ethoxy)-2,3-dimethyl-phenyl]-octahydro-pyrido[1,2-a]-pyrazin-2-yl}-(6-trifluoromethyl-pyridin-3-yl)-methanone

A solution of [(6R,9aS)-6-(4-hydroxy-2,3-dimethylphenyl)-octahydro-pyrido[1,2-a]pyrazin-2-yl]-(6-trifluoromethyl-pyridin-3-yl)-methanone (1.54 g, 3.55 mmol) in CH₃CN is treated with powdered KOH (400 mg, 7.10 mmol, 1.5 eq) and 2-bromoethyl methyl ether (500 μL, 5.33 mmol, 2.0 eq) and heated in a sealed tube reactor with stirring in a 60° C. oil bath for 20.5 h. After cooling, the mixture is filtered through a celite pad. The pad is washed with DCM and the solution is concentrated in vacuo. The residue is purified by flash chromatography on silica gel eluting with 50-40% hexanes/EtOAc to yield {(6R,9aS)-6-[4-(2-methoxy-ethoxy)-2,3-dimethyl-phenyl]-octahydro-pyrido[1,2-a]pyrazin-2-yl}-(6-trifluoro-methyl-pyridin-3-yl)-methanone as a white foam with the following physical properties: LC/MS: 492 (M+1). ¹H NMR (mixture of rotamers, 400 MHz, CDCl₃): 8.73 (1H, d), 7.90 (1H, dd), 7.88 (1H, dd), 7.30 (1H, dd), 6.73 (1H, dd), 4.50 (1H, dd), 4.10 (2H, dd), 3.76 (2H, m), 3.46-3.30 (5H, bm), 3.20-3.02 (1H, bm), 2.91-2.51 (3H, bm), 2.25 (6H, m), 1.87-1.72 (4H, bm), 1.40-1.32 (3H, bm). The material is dissolved in EtOAc, treated with one equivalent of HCl (1M in diethyl ether) and allowed to stand for 10 min. The mixture is concentrated in vacuo to afford the title product (monohydrochloride salt) as a white solid.

### Example 2

### {(6R,9AS)-6-[4-(2-HYDROXY-ETHOXY)-2,3-DIMETHYL-PHENYL]-OCTA-HYDRO-PYRIDO[1,2-A]PYRAZIN-2-YL}-(6-TRIFLUOROMETHYL-PYRIDIN-3-YL)-METHANONE

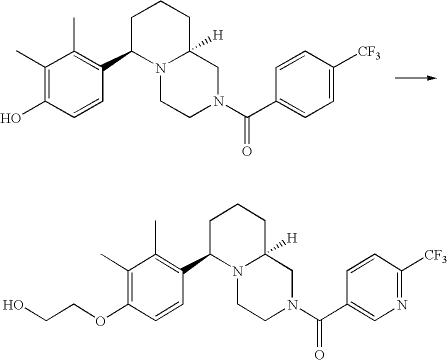

### Step 1. ((6R,9aS)-6-{4-[2-(tert-butyl-dimethyl-silanyloxy)-ethoxy]-2,3-dimethyl-phenyl}-octahydro-pyrido[1,2-a]pyrazin-2-yl)-(6-trifluoromethyl-pyridin-3-yl)-methanone

A solution of [(6R,9aS)-6-(4-hydroxy-2,3-dimethylphenyl)-octahydro-pyrido[1,2-a]pyrazin-2-yl]-(6-trifluoromethyl-pyridin-3-yl)-methanone (100 mg, 0.23 mmoles, Example 1) in acetonitrile is treated with powdered KOH (26 mg, 0.461 mmoles, 2.0 equiv.) and (2-bromoethoxy)-tert-butyldimethylsilane (50 μL, 0.35 mmoles, 1.5 equiv.) and heated in a sealed tube with stirring in a 60° C. oil bath for 7 h and then allowed to stand at ambient temperature for 19 h. The mixture is filtered through a celite pad, the pad is washed with dichloromethane and the solution is concentrated in vacuo. The residue is purified by PTLC on a 2 mm silicagel plate eluting with 60% hexanes/EtOAc to yield ((6R,9aS)-6-{4-[2-(tert-butyl-dimethyl-silanyloxy)-ethoxy]-2,3-dimethylphenyl}-octahydro-pyrido[1,2-a]pyrazin-2-yl)-(6-trifluoromethyl-pyridin-3-yl)-methanone as a white foam. LC/MS: 592 (M+1).

### Step 2. ((6R,9aS)-6-[4-(2-hydroxy-ethoxy)-2,3-dimethyl-phenyl]-octahydro-pyrido[1,2-a]pyrazin-2-yl)-(6-trifluoromethyl-pyridin-3-yl)-methanone

A solution of the TBDMS-ether from step 1 (117 mg) is dissolved in 3.0 mL of anhydrous THF, cooled to 0° C. under N₂, treated with tetra-n-butyl ammonium fluoride (1M in THF, 250 μL) and stirred at that temperature for 15 min. Analysis by TLC and LC/MS indicates consumption of starting material. The reaction is quenched by the addition of brine and extracted with EtOAc. The combined extracts are dried over Na₂SO₄, filtered and concentrated in vacuo. Purification by preparative TLC on a 2 mm silicagel plate eluting with 60% hexanes/EtOAc yields the desired product as a white foam. ¹H NMR (mixture of rotamers, 400 MHz, CDCl₃): 8.74 (1H, d), 7.91 (1H, dd), 7.73 (1H, dd), 7.34 (1H, dd), 6.73 (1H, dd), 4.52 (1H, dd), 4.06-3.90 (4H, m), 3.42-2.52 (6H, bm), 2.52-1.18 (15H, bm). LC/MS: 478 (M+1). The material is dissolved in DCM, treated with one equivalent of HCl (1M in Et₂O) and allowed to stand for 10 min at room temperature. Concentration in vacuo yields the title product, monohydrochloride salt as a white amorphous solid.

### Example 3

### {(6R,9AS)-6-[4-((S)-2-HYDROXY-PROPOXY)-2,3-DIMETHYL-PHENYL]-OCTAHYDRO-PYRIDO[1,2-A]PYRAZIN-2-YL}-(6-TRIFLUOROMETHYL-PYRIDIN-3-YL)-METHANONE

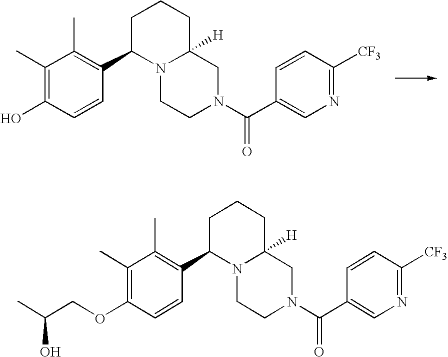

Using the protocols described in Example 2, steps 1 and 2, replacing (2-bromoethoxy)-tert-butyldimethylsilane with an equivalent amount of toluene-4-sulfonic acid (S)-2-(tert-butyl-dimethyl-silanyloxy)-propyl ester (obtained as described in J. Nat. Prod. 64:472-479 (2001)), the title product is obtained as an oil. ¹H NMR (CDCl₃): 8.74 (d, 1H), 7.92 (dd, 1H), 7.74 (dd, 1H), 7.33 (dt, 1H), 6.72 (dd, 1H), 4.52 (dd, 1H), 4.20 (br s, 1H), 3.94-3.88 (m, 1H), 3.82-3.74 (m, 1H), 3.42-3.32 (m, 2H), 3.23-3.04 (m, 1H), 2.92-2.53 (m, 4H), 2.20 (s, 6H), 1.89-1.70 (m, 4H), 1.49-1.42 (m, 2H), 1.29 (s, 3H). LC/MS: 492 (M+1).

### Example 4

### {(6R,9AS)-6-[4-((R)-2-HYDROXY-PROPOXY)-2,3-DIMETHYL-PHENYL]-OCTAHYDRO-PYRIDO[1,2-A]PYRAZIN-2-YL}-(6-TRIFLUOROMETHYL-PYRIDIN-3-YL)-METHANONE

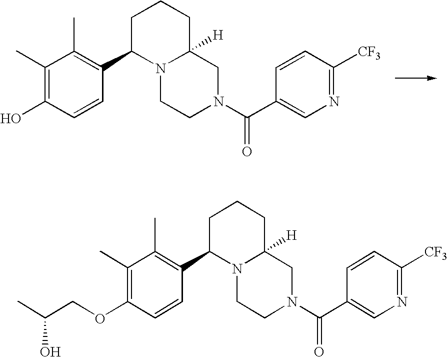

Using the protocol illustrated in Example 3, replacing toluene-4-sulfonic acid (S)-2-(tert-butyl-dimethyl-silanyloxy)-propyl ester with an equivalent amount of toluene-4-sulfonic acid (R)-2-(tert-butyl-dimethyl-silanyloxy)-propyl ester (obtained as described in J. Nat. Prod. 64:472-479 (2001)), the title product is obtained as an oil. ¹H NMR (CDCl₃): 8.75 (d, 1H), 7.92 (dd, 1H), 7.74 (dd, 1H), 7.33 (dt, 1H), 6.72 (dd, 1H), 4.53 (dd, 1H), 4.19 (br s, 1H), 3.94-3.88 (m, 1H), 3.81-3.74 (m, 1H), 3.40-3.32 (m, 2H), 3.23-3.04 (m, 1H), 2.91-2.53 (m, 4H), 2.22 (s, 6H), 1.89-1.70 (m, 4H), 1.47-1.33 (m, 2H), 1.29 (s, 3H). LC/MS: 492 (M+1).

### Example 5

### 1-{2,3-DIMETHYL-4-[(6R,9AS)-2-(6-TRIFLUOROMETHYL-PYRIDINE-3-CARBONYL)-OCTAHYDRO-PYRIDO[1,2-A]PYRAZIN-6-YL]-PHENOXY}-PROPAN-2-ONE

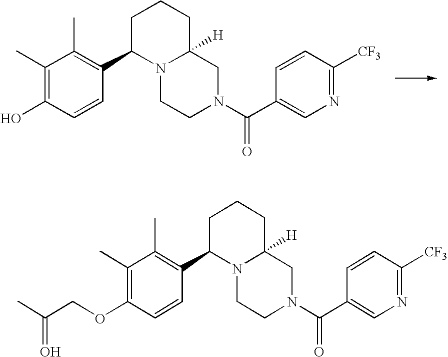

Using the protocol illustrated in Example 4, replacing ((S)-2-bromo-1-methyl-ethoxy)-tert-butyl-dimethyl-silane with excess of chloroacetone, the title product is obtained as an oil. LC/MS: 490 (M+1).

### Example 6

### 1-{2,3-DIMETHYL-4-[(6R,9AS)-2-(6-TRIFLUOROMETHYL-PYRIDINE-3-CARBONYL)-OCTAHYDRO-PYRIDO[1,2-A]PYRAZIN-6-YL]-PHENOXY}-PROPAN-2-ONE OXIME

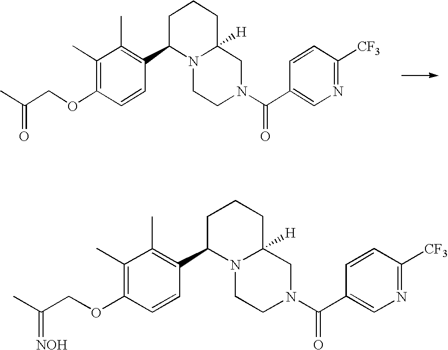

1-{2,3-Dimethyl-4-[(6R,9aS)-2-(6-trifluoromethyl-pyridine-3-carbonyl)-octahydro-pyrido[1,2-a]pyrazin-6-yl]-phenoxy}-propan-2-one is treated with an excess of NH₂OH.HCl in MeOH in the presence of 3 equivalents of NaOAc at room temperature for 16 h. After concentrating the reaction mixture to dryness under reduced pressure, a white solid is obtained. This is partitioned between EtOAc and brine, and the organic layer is dried over Na₂SO₄ and evaporated under reduced pressure to produce a quantitative yield of the title compound as a white solid. LC/MS: 505 (M+1).

### Example 7

### (6-CHLOROPYRIDIN-3-YL)-((1S,4S)-5-{(S)-1-[4-(2-METHOXY-ETHOXY)-2,3-DIMETHYLPHENYL]-ETHYL}-2,5-DIAZABICYCLO[2.2.1]HEPT-2-YL)-METHANONE

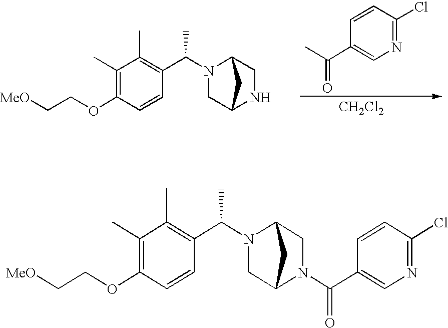

Aqueous NaHCO₃ (saturated solution, 3 mL) is slowly added to a mixture of (1S,4S)-2-{(S)-1-[4-(2-methoxy-ethoxy)-2,3-dimethylphenyl]-ethyl}-2,5-diazabicyclo[2.2.1]heptane in 5 mL of DCM. The mixture is stirred vigorously at room temperature for 1 h, and is then diluted with 1N NaOH (5 mL) and extracted with DCM (2×25 mL). The combined extracts are dried over Na₂SO₄, filtered and concentrated under reduced pressure. The crude material is purified by flash chromatography on silicagel, eluting with CHCl₃—MeOH (40:1 to 20:1) to afford the title compound as a clear oil. LC/MS: 444 (M+1).

### Example 8

### (6-ETHYLPYRIDIN-3-YL)-((1S,4S)-5-{(S)-1-[4-(2-METHOXY-ETHOXY)-2,3-DIMETHYLPHENYL]-ETHYL}-2,5-DIAZABICYCLO[2.2.1]HEPT-2-YL)-METHANONE

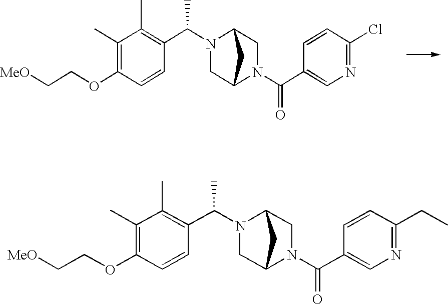

5 mg of Fe(acac)₃ followed by EtMgBr (0.73 mL, 1N in THF) is added to a solution of 6-chloropyridin-3-yl)-((1S,4S)-5-{(S)-1-[4-(2-methoxy-ethoxy)-2,3-dimethylphenyl]-ethyl}-2,5-diazabicyclo[2.2.1]hept-2-yl)-methanone (Example 3, 129 mg) dissolved in 3 mL of THF and 0.3 mL of N-methylpyrrolidinone at room temperature under N₂. The dark purple reaction mixture is stirred at room temperature for 50 min and then diluted with brine and extracted 3 times with EtOAc (10 mL). The combined extracts are dried over Na₂SO₄, filtered, and concentrated under reduced pressure. The crude product is purified by silicagel PTLC, developing 2 times with CHCl₃—MeOH (25:1) to afford the title product as a clear oil. LC/MS: 438 (M+1).

### Example 9

### [(6R,8AS)-6-(4-METHOXY-2,3-DIMETHYLPHENYL)-HEXAHYDRO-PYRROLO[1,2-A]PYRAZIN-2-YL]-(6-TRIFLUOROMETHYL-PYRIDIN-3-YL)-METHANONE

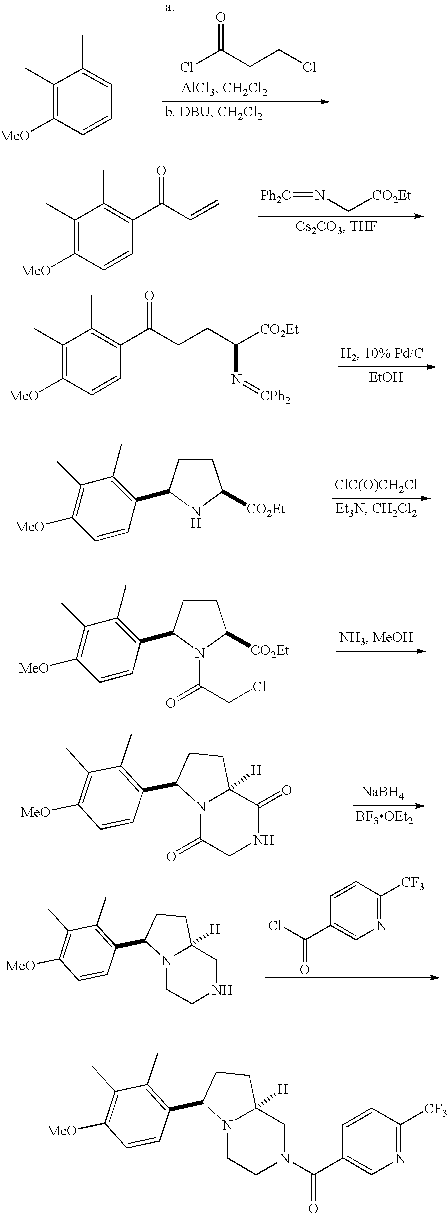

### Step 1. 3-Chloro-1-(4-methoxy-2,3-dimethylphenyl)propan-1-one

3-Chloropropionyl chloride (12.70 g, 100 mmol) is slowly added to a suspension of AlCl₃ (16.0 g, 120 mmol) in DCM (200 mL) at 0° C. under N₂. Next, 2,3-dimethylanisole (13.62 g, 100 mmol) is slowly added at 0° C. The resulting yellow solution is stirred at 0° C. for 30 min., and then quenched by the addition of ice-cold 1.0 N HCl (200 mL) (the first several mL are added very slowly). The resulting mixture is stirred at room temperature for 20 min and then extracted with DCM. The extract is washed again with water (100 mL) and brine (100 mL), dried over Na₂SO₄ and concentrated in vacuo to a yield white solid. ¹H NMR (CDCl₃, 400 MHz): 7.50 (d, J=8.6 Hz, 1H), 6.74 (d, J=8.6 Hz, 1H), 3.90 (t, J=6.8 Hz, 2H), 3.87 (s, 3H), 3.34 (t, J=6.8 Hz, 2H), 2.41 (s, 3H), 2.18 (s, 3H).

### Step 2. 1-(4-Methoxy-2,3-dimethylphenyl)propenone

The crude 3-chloro-1-(4-methoxy-2,3-dimethylphenyl)propan-1-one is redissolved in DCM (200 mL). The resulting solution is cooled to 0° C. and treated with DBU (15.0 mL, 100 mmol). After 30 min, additional DBU (0.75 mL, 5 mmol) is added. After an additional 15 min, the reaction mixture is concentrated in vacuo. The residue is partitioned between Et₂O and water (150 mL). The layers are separated, and the Et₂O extract is washed with additional water (100 mL) and brine (100 mL). The aqueous washes are reextracted once with Et₂O and the combined extracts are dried over Na₂SO₄ and concentrated to a light yellow oil. ¹H NMR (CDCl₃, 400 MHz): 7.33 (d, J=8.4 Hz, 1H), 6.78 (dd, J=17.4, 10.6 Hz, 1H), 6.73 (d, J=8.4 Hz, 1H), 6.14 (dd, J=17.4, 1.4 Hz, 1H), 5.94 (dd, J=10.4, 1.6 Hz, 1H), 3.86 (s, 3H), 2.33 (s, 3H, 2.18 (s, 3H).

### Step 3. 2-(benzhydrylideneamino)-5-(4-methoxy-2,3-dimethylphenyl)-5-oxopentanoic acid ethyl ester

Cs₂CO₃ (0.51 g, 1.58 mmol) is added to a solution of 1-(4-methoxy-2,3-dimethylphenyl)-propenone (3.15 g, 16.56 mmol) and N-(diphenylmethylene)glycine ethyl ester (4.22 g, 15.77 mmol) in THF (40 mL) at 0° C. After 5 min, the ice bath is removed and the reaction mixture is stirred at room temperature overnight. The reaction mixture is then diluted with Et₂O and washed with water (1×50 mL) and brine (1×50 mL). The aqueous washes are reextracted once with Et₂O, and the combined extracts are dried over Na₂SO₄ and concentrated. The crude oil is purified by flash column chromatography on silica gel. Elution with 4:1 hexanes-EtOAc affords 2-(benzhydrylideneamino)-5-(4-methoxy-2,3-dimethylphenyl)-5-oxopentanoic acid ethyl ester as a colorless syrup. ¹H NMR (CDCl₃, 400 MHz): 7.64 (m, 2H), 7.48 (d, J=8.8 Hz, 1H), 7.43-7.37 (m, 4H), 7.32 (m, 2H), 7.15 (m, 2H), 6.69 (d, J=8.8 Hz, 1H), 4.20-4.13 (m, 3H), 3.85 (s, 3H), 2.93 (t, J=7.6 Hz, 2H), 2.31 (m, 2H), 2.31 (s, 3H), 2.16 (s, 3H), 1.25 (t, J=7.0 Hz, 3H). LC/MS: 458 (M+1).

### Step 4. cis-5-(4-Methoxy-2,3-dimethylphenyl)pyrrolidine-2-carboxylic acid ethyl ester

A solution of 2-(benzhydrylideneamino)-5-(4-methoxy-2,3-dimethylphenyl)-5-oxopentanoic acid ethyl ester (16.56 mmol) in EtOH (80 mL) containing 10% Pd/C (760 mg) is stirred under 1 atm of H₂ (double-stuffed balloon) for 18 h. The reaction mixture is then filtered through of pad of celite using MeOH for the rinse. The filtrated is concentrated in vacuo to a nearly colorless syrup, which is used in the next reaction without further purification. ¹H NMR (CDCl₃, 400 MHz): 7.46 (d, J=8.6 Hz, 1H), 6.75 (d, J=8.6 Hz, 1H), 4.40 (dd, J=8.8, 6.6 Hz, 1H), 4.23 (q, J=8.8 Hz, 2H), 3.90 (dd, J=8.6, 5.4 Hz, 1H), 3.82 (s, 3H), 2.29 (s, 3H), 2.19 (s, 3H), 2.25-2.05 (m, 4H), 1.72-1.65 (m, 1H), 1.31 (t, J=8.8 Hz, 3H). LC/MS: 278 (M+1).

### Step 5. cis-1-(2-Chloroacetyl)-5-(4-methoxy-2,3-dimethylphenyl)pyrrolidine-2-carboxylic acid ethyl ester

Chloroacetyl chloride (1.7 mL, 21.5 mmol) is added to a solution of cis-5-(4-methoxy-2,3-dimethylphenyl)pyrrolidine-2-carboxylic acid ethyl ester (16.56 mmol) and Et₃N (3.5 mL, 24.8 mmol) in DCM (80 mL) at 0° C. The reaction mixture is stirred at 0° C. for 15 min and then at room temperature for 45 min. The mixture is then poured into half-saturated aq. NaHCO₃ (100 mL) and extracted with EtOAc. The extract is further washed with water (1×50 mL) and brine (1×50 mL). The aqueous washes are reextracted once with EtOAc and the combined extracts are dried over Na₂SO₄ and concentrated. The crude material is used in the next step without further purification. ¹H NMR (CDCl₃, 400 MHz): 7.90 (d, J=8.6 Hz, 1H), 6.78 (d, J=8.6 Hz, 1H), 5.30 (dd, J=7.6, 3.6 Hz, 1H), 4.53 (t, J=8.0 Hz, 1H), 4.37-4.21 (m, 2H), 3.81 (s, 3H), 3.77, 3.65 (ABq, J=13.2 Hz, 2H), 2.50-2.41 (m, 1H), 2.26 (s, 3H), 2.24-2.14 (m, 1H), 2.19 (s, 3H), 2.09-2.00 (m, 1H), 1.96-1.89 (m, 1H), 1.35 (t, J=7.2 Hz, 3H). LC/MS: 354 (M+1).

### Step 6. cis-6-(4-Methoxy-2,3-dimethylphenyl)hexahydropyrrolo[1,2-a]pyrazine-1,4-dione

A mixture of the crude cis-1-(2-chloroacetyl)-5-(4-methoxy-2,3-dimethylphenyl)pyrrolidine-2-carboxylic acid ethyl ester (approximately 16.6 mmol) and ca. 7M NH₃ in MeOH (50 mL) is stirred in a sealed flask at room temperature for 2.5 days. The mixture is then diluted with water (ca. 200-300 mL). The resulting suspension is cooled to 0° C. and stirred well. The mixture is then filtered and the solid thoroughly washed with water, followed by Et₂O. Drying affords cis-6-(4-methoxy-2,3-dimethylphenyl)-hexahydropyrrolo[1,2-a]pyrazine-1,4-dione as a slightly off-white powder. ¹H NMR (CDCl₃, 400 MHz): 6.70 (br, 1H), 6.68 (d, J=8.6 Hz, 1H), 6.64 (d, J=8.6 Hz, 1H), 5.38 (d, J=8.8 Hz, 1H), 4.29 (dd, J=10.8, 6.4 Hz, 1H), 4.10, 3.93 (ABXq, J_{AB}=16.8 Hz, J_{AX}=1.0 Hz, J_{BX}=4.8 Hz, 2H), 3.77 (s, 3H), 2.43-2.31 (m, 1H), 2.26 (s, 3H), 2.24-2.11 (m, 2H), 2.16 (s, 3H), 1.85 (dd, J=12.2, 5.8 Hz, 1H). LC/MS: 289 (M+1).

### Step 7. cis-6-(4-Methoxy-2,3-dimethylphenyl)octahydropyrrolo[1,2-a]pyrazine

The diketopiperazine from step 6 is dissolved in 1,2-dimethoxyethane (30 mL) at room temperature. NaBH₄ (0.158 g, 4.18 mmol) is added in one portion, followed by BF₃.OEt₂ (350 μL, 2.51 mmol). The mixture is heated at reflux temperature (ca. 90° C.) for 3 h and then cooled to 0° C. The reaction is quenched by addition of MeOH (50 mL) and then HCl (conc., 35 mL). The resulting solution is stirred at room temperature for 20 min and then at reflux temperature for 45 min. The organic solvents are evaporated under reduced pressure and the residue is taken with NaOH 1N. Extractive work-up with EtOAc washing with brine, drying with MgSO₄, filtration and concentration under reduced pressure affords the desired amine as an oil. Purification is carried out by flash chromatography on silicagel eluting with EtOAc to produce the title compound as a while solid. ¹H NMR (400 MHz, CDCl₃): 7.3 (br, 1H), 6.7 (br, 1H), 4.8 (br, 1H), 3.8 (s, 3H), 3.6 (br, 1H), 3.4 (d, 1H), 3.2 (d, 1H), 2.9 (m, 2H), 2.8 (t, 1H), 2.4 (br, 1H), 2.1-2.3 (m, 8H), 1.9 (m, 1H), 1.5 (m, 1H). LC/MS: 261 (M+1).

### Step 8. [(6,8a)-6-(4-Methoxy-2,3-dimethylphenyl)-hexahydro-pyrrolo[1,2-a]pyrazin-2-yl]-(6-trifluoromethyl-pyridin-3-yl)-methanone

6-Trifluoromethyl nicotinic acid (18.1 mg, 0.12 mmol), BOP (66.3 mg, 0.15 mmol), and NEt₃ (34.8 μL, 0.25 mmol) are added to a solution of (6R,8aS)-6-(4-methoxy-2,3-dimethylphenyl)-octahydro-pyrrolo[1,2-a]pyrazine (52.2 mg, 0.2 mmol) in anhydrous DMA (0.1 mL). The reaction mixture is stirred at 50° C. for 16 h, diluted with toluene, evaporated to dryness and the residue purified by filtration through an SCX cartridge, eluting with EtOAc—MeOH—NEt₃ (10-1-1) to produce an oil (LC/MS: 434).

### Example 10

### [2-(2-CHLORO-5-{4-[1-(3,4-DIMETHOXYPHENYL)-ETHYL]-PIPERAZIN-1-YL}-PHENOXY)-ETHYL]-DIMETHYL-AMINE

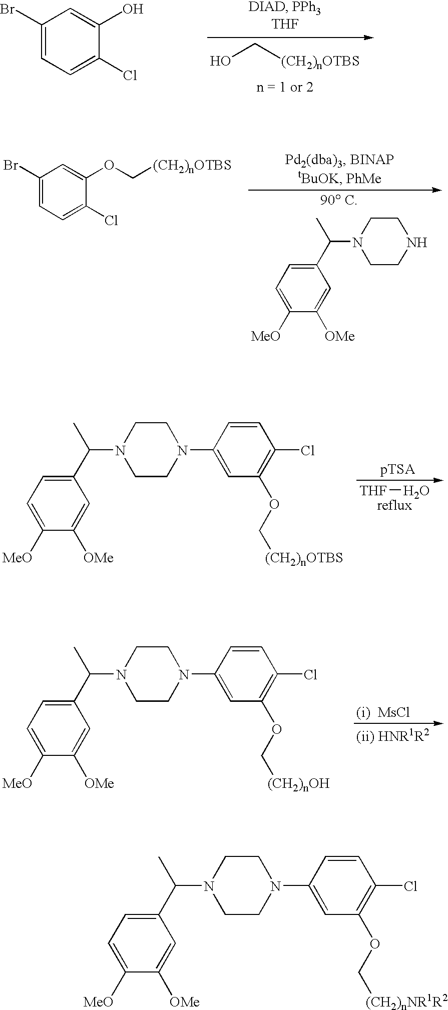

### Step 1. [2-(5-Bromo-2-chloro-phenoxy)-ethoxy]-tert-butyldimethylsilane

5-Bromo-2-chlorophenol (4.14 g, 20 mmol) and then tert-(butyldimethylsilyloxy)ethanol (3.8 g, 20 mmol) are added to a solution of diisopropyl azodicarboxylate (4.04 g, 20 mmol) and PPh₃ (5.26 g, 20 mmol) in THF (200 ml) at 0° C. The reaction mixture is allowed to return to room temperature and stirred overnight. The residue is partitioned between EtOAc and 1M NaOH and further extracted with EtOAc. The combined extracts are dried (MgSO₄) and concentrated under reduced pressure. The residue is purified by flash chromatography on silica gel (90% hexane/10% ether) to give the title compound. LC/MS: 367 (M+1), 389 (M+23).

### Step 2. 1-{3-[2-(tert-Butyldimethylsilanyloxy)-ethoxy]-4-chloro-phenyl}-4-[1-(3,4-dimethoxy-phenyl)-ethyl]-piperazine

(3,4-Dimethoxyphenyl)-ethyl-piperazine (1.62 g, 6.5 mmol) followed by potassium tert-butoxide (3.7 g, 33 mmol) are added to a solution of [2-(5-bromo-2-chloro-phenoxy)-ethoxy]-tert-butyldimethylsilane (2.0 g, 5.5 mmol), Pd₂(dba)₃ (594 mg, 0.66 mmol), and BINAP (550 mg, 0.88 mmol) in toluene (75 mL) under nitrogen. The mixture is heated at 90° C. for 2 h, diluted with aqueous ammonium chloride, and extracted with EtOAc. The combined extracts are dried (MgSO₄) and concentrated under reduced pressure. The residue is purified by flash chromatography on silica gel (EtOAc) to give the title compound. LC/MS: 535 (M+1).

### Step 3. 2-(2-Chloro-5-{4-[ ]-(3,4-dimethoxy-phenyl)-ethyl]-piperazin-1-yl}-phenoxy)-ethanol

1-{3-[2-(Tert-butyldimethylsilanyloxy)-ethoxy]-4-chloro-phenyl}-4-[1-(3,4-dimethoxyphenyl)-ethyl]-piperazine (2.0 g, 3.7 mmol) and p-toluenesulfonic acid (200 mg) are mixed in THF:water (100 ml, 4:1) and heated at reflux for 48 h. The residue is partititioned between EtOAc and NaHCO₃ solution and extracted with further EtOAc. The combined extracts are dried (MgSO₄) and concentrated under reduced pressure. The residue is purified by flash chromatography on silica gel (10% MeOH/90% dichloromethane) to give the title compound. LC/MS: 421 (M+1).

### Step 4. 2-(2-Chloro-5-{4-[1-(3,4-dimethoxy-phenyl)-ethyl]-piperazin-1-yl}-phenoxy)-ethyl]-dimethyl amine

2-(2-Chloro-5-{4-[1-(3,4-dimethoxy-phenyl)-ethyl]-piperazin-1-yl}-phenoxy)-ethanol (84 mg, 0.2 mmol) and dry NEt₃ (22 mg, 0.2 mmol) are mixed in DCM (4 mL) and methanesulfonyl chloride (24 mg, 0.2 mmol) is added. The solution is stirred at room temperature for 1 h and evaporated to dryness. The residue is re-dissolved in acetonitrile (3 mL), transferred to a sealed tube, potassium carbonate (55 mg, 0.4 mmol) and DMA (1 mmol) are added and the mixture is heated at 80° C. for 8 h. The residue is partitioned between EtOAc and NaHCO₃ solution and extract with further EtOAc. The combined extracts are dried (MgSO₄) and concentrated under reduced pressure. The residue is purified by flash chromatography on silica gel (5% MeOH/95% DCM) to yield the title compound. LC/MS: 449 (M+1).

### Example 11

### (6R,9AS)-2-(4-CHLORO-3-METHOXYPHENYL)-6-[2,3-DIMETHYL-4-(3-MORPHOLIN-4-YL-PROPOXY)-PHENYL]-OCTAHYDRO-PYRIDO[1,2-A]PYRAZINE

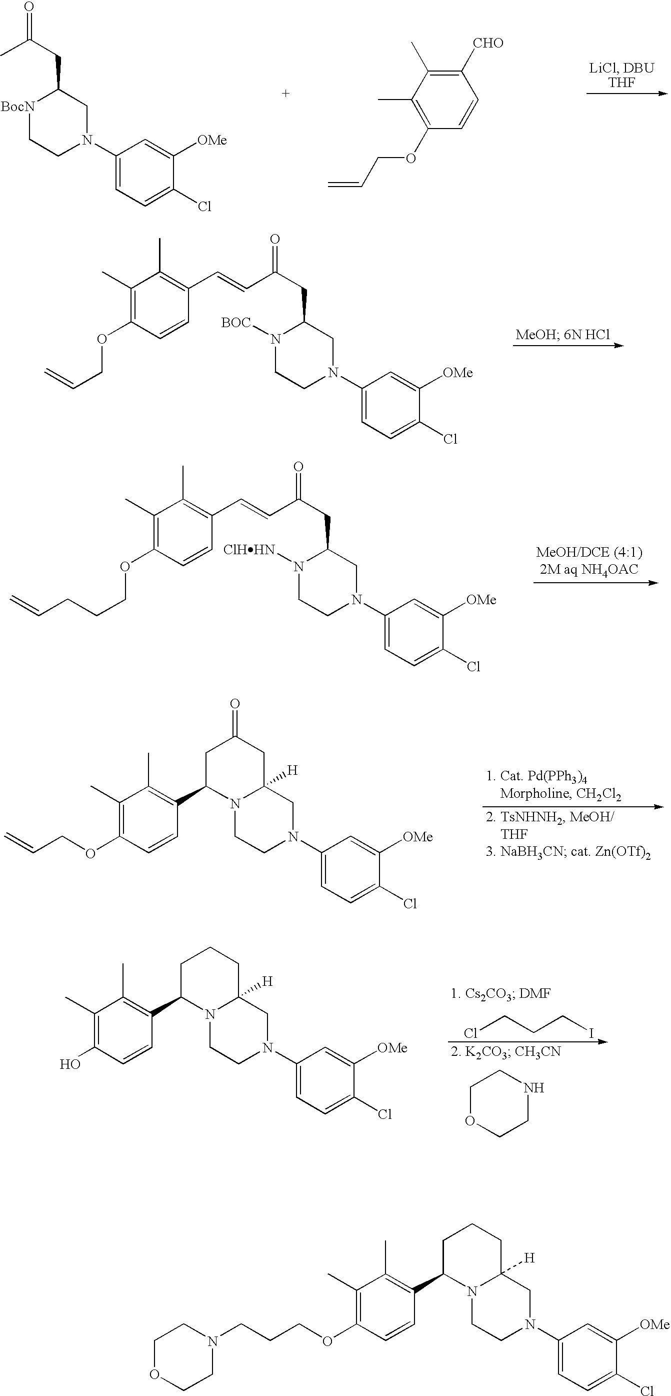

### Step 1. 2-[4-(4-Allyloxy-2,3-dimethyl-phenyl)-2-oxo-but-3-enyl]-4-(4-chloro-3-methoxy-phenyl)-piperazine-1-carboxylic acid tert-butyl ester

To a cooled (0° C.) solution of 4-(4-chloro-3-methoxy-phenyl)-2-(2-oxo-propyl)-piperazine-1-carboxylic acid tert-butyl ester (obtained as described in PCT International Publication No. WO 02/094799, page 57; 17 g, 0.044 mol) and 4-allyloxy-2,3-dimethyl-benzaldehyde (9.3 g, 0.048 mol) in anhydrous THF (200 mL), is added anhydrous LiCl (9.4 g, 0.22) with stirring. The reaction mixture is stirred for 45 min to dissolve most of the LiCl. DBU (6.65 mL, 0.048 mol) is added dropwise to the above mixture and stirring is continued overnight at room temperature. The reaction is quenched by pouring onto ice-cold water (300 mL), and is then partitioned with EtOAc. The organic layer is washed with water, followed by brine, dried over Na₂SO₄, and concentrated under vacuum to obtain the title product. LC-MS: 556 (M+1).

### Step 2. 4-(4-Allyloxy-2,3-dimethylphenyl)-1-[4-(4-chloro-3-methoxy-phenyl)-piperazin-2-yl]-but-3-en-2-one, hydrochloride salt

The crude product from step 1 (24 g, 0.043 mol) is dissolved in 200 mL of MeOH, and 30 mL of 6 N HCl is added. The reaction mixture is heated at 60° C. for 3 h, cooled to room temperature and concentrated under reduced pressure. Water is removed from this crude product by taking it to dryness under reduced pressure twice in the presence of added toluene. Then it is triturated with Et₂O dried under high vacuum to remove traces of solvents from the title product. LC/MS: 458 (M+1).

### Step 3. 6-(4-Allyloxy-2,3-dimethylphenyl)-2-(4-chloro-3-methoxyphenyl)-octahydro-pyrido[1,2-a]pyrazin-8-one

To a solution of the HCl salt from step 2 (19.5 g) in a mixture of MeOH (400 mL) and dichloroethane (100 mL) is added an aqueous solution of ammonium acetate (210 mL, 2M). The resulting suspension is stirred overnight at 60° C. The reaction mixture is cooled to 0° C. and quenched by addition of NaOH (1N, 100 mL), stirring for 15 min. It is then concentrated under reduced pressure and the residue partitioned with EtOAc. After washing the organic layer with brine and drying over Na₂SO₄, the organic residue is submitted to flash chromatography over silicagel eluting with 25% EtOAc-hexanes to afford the title product as an oil. ¹H NMR (300 MHz, CDCl₃): 7.18 (d, J=8.7 Hz, 2H), 6.71-6.82 (m, 1H), 6.39-6.47 (m, 2H), 6.02-6.16 (m, 1H), 5.35 (dd, J=33, 15 Hz, 2H), 4.53 (d, J=6.3 Hz, 2H), 3.85 (s, 3H), 3.81-3.88 (m, 1H), 3.45-3.50 (m, 2H), 2.65-2.93 (m, 5H), 2.50-2.59 (m, 2H), 2.35-2.48 (m, 2H), 2.21 (s, 6H). LC/MS: 456 (M+1).

### Step 4. 2-(4-chloro-3-methoxyphenyl)-6-(4-hydroxy-2,3-dimethylphenyl)-octahydro-pyrido[1,2-a]pyrazin-8-one

To a solution of 6-(4-allyloxy-2,3-dimethylphenyl)-2-(4-chloro-3-methoxyphenyl)-octahydro-pyrido[1,2-a]pyrazin-8-one (9.5 g, 0.02 mol) in anhydrous DCM (100 mL) are added morpholine (2 mL, 0.022 mol) and tetrakis(triphenylphosphine) palladium (0) (0.7 g, 0.6 mmol) under an argon atmosphere. The reaction mixture is stirred for 1 h at room temperature, concentrated under reduced pressure and submitted to flash chromatography over silica gel eluting with 40% EtOAc-hexanes to afford the title product. ¹H NMR (300 MHz, CDCl₃): 6.99 (d, J=8.7 Hz, 2H), 6.51-6.62 (m, 1H), 6.22-6.29 (m, 2H), 3.69 (s, 3H), 3.42-3.60 (m, 1H), 3.20-3.41 (m, 2H), 2.51-2.85 (m, 5H), 2.30-2.41 (m, 2H), 1.96-2.24 (m, 2H), 1.99 (s, 6H); LC/MS: 416 (M+1)

### Step 5. 4-[2-(4-Chloro-3-methoxyphenyl)-octahydro-pyrido[1,2-a]pyrazin-6-yl]-2,3-dimethyl-phenol

To a solution of 2-(4-chloro-3-methoxyphenyl)-6-(4-hydroxy-2,3-dimethylphenyl)-octahydro-pyrido[1,2-a]pyrazin-8-one (8.1 g, 0.019 mol) in a mixture of anhydrous THF (100 mL) and MeOH (50 mL) is added TsNHNH₂ (4.2 g, 0.02 mol) under an argon atmosphere. The reaction mixture is stirred overnight at room temperature. Argon is bubbled through the reaction mixture for 15 min and NaCNBH₃ (3.85 g, 0.06 mol) is added followed by addition of Zn(OTf)₂ (0.15 g, 0.4 mmol). The resulting reaction mixture is stirred at 65° C. for 5h, cooled to room temperature and quenched by addition of a saturated solution of NaHCO₃ (200 ml) and stirring for 15 min. The volatiles are evaporated under reduced pressure and the organic residue is partitioned with EtOAc and brine, dried over Na₂SO₄ and concentrated under reduced pressure. The crude product is submitted to flash chromatography over silica gel eluting with 60% EtOAc-hexanes to afford the title product. ¹H NMR (300 MHz, CDCl₃): 7.29 (d, J=6.3 Hz, 1H), 7.17 (d, J=6.3 Hz, 1H), 6.64 (d, J=6.0 Hz, 1H), 6.46 (d, J=2.1 Hz, 1H), 6.42 (dd, J=6.6, 1.8 Hz, 1H), 4.8 (br, 1H), 3.86 (s, 3H), 3.44 (d, J=8.4 Hz, 1H), 3.32 (t, J=7.8 Hz, 2H), 2.72-2.81 (m, 2H), 2.62 (t, J=8.1 Hz, 2H), 2.31-2.35 (m, 1H), 2.23 (s, 3H), 2.19 (s, 3H), 1.81-2.02 (m, 2H), 1.66-1.72 (m, 2H), 1.41-1.50 (m, 2H); LC/MS: 401 (M+1).

### Step 6. Preparation of (6R,9aS)-2-(4-chloro-3-methoxy-phenyl)-6-[4-(3-chloro-propoxy)-2,3-dimethyl-phenyl]-octahydro-pyrido[1,2-a]pyrazine

To a solution of 4-[(6R,9aS)-2-(4-chloro-3-methoxyphenyl)-octahydro-pyrido[1,2-a]pyrazin-6-yl]-2,3-dimethylphenol (1.54 g, 3.84 mmol) in DMF (19 mL) at room temperature is added Cs₂CO₃ (1.50 g, 4.61 mmol). The mixture is stirred at room temperature for 15 min before 1-chloro-3-iodopropane (0.61 mL, 5.76 mmol) is added. The mixture is stirred at room temperature overnight and then diluted with water (30 mL) and extracted with EtOAc. The organic extract is washed with additional water (30 mL) and then with brine (30 mL). The aqueous washes are reextracted once with EtOAc, and the combined extracts are dried over Na₂SO₄ and concentrated. The residue is purified by flash chromatography on silica gel. Elution with 4:1 hexanes-EtOAc followed by 2:1 hexanes-EtOAc and finally 1:1 hexanes-EtOAc affords the title product as a colorless foam. ¹H NMR (CDCl₃, 400 MHz): 7.38 (d, J=8.4 Hz, ˜0.8H), 7.18 (d, J=8.8 Hz, 1H), 6.90 (br, ˜0.2H), 6.75 (d, 8.4 Hz, ˜0.8H), 6.63 (br, ˜0.2H), 6.46 (d, J=2.8 Hz, 1H), 6.42 (dd, J=8.8, 2.4 Hz, 1H), 4.09 (t, J=5.2 Hz, 2H), 3.86 (s, 3H), 3.78 (t, J=6.6 Hz, 2H), 3.45 (br d, J=11.6 Hz, 1H), 3.34 (m, ˜2H), 3.08 (br, ˜0.2H), 2.82-2.55 (m), 2.35 (br t), 2.28-2.22 (m), 2.24 (s, 3H), 2.18 (s, 3H), 1.98 (m), 1.82 (m), 1.68 (m), 1.48 (m) ppm. LC/MS: 477 (M+1).

### Step 7. Preparation of (6R,9aS)-2-(4-chloro-3-methoxyphenyl)-6-[2,3-dimethyl-4-(3-morpholin-4-yl-propoxy)-phenyl]-octahydro-pyrido[1,2-a]pyrazine

A solution of (6R,9aS)-2-(4-chloro-3-methoxyphenyl)-6-[4-(3-chloropropoxy)-2,3-dimethyl-phenyl]-octahydro-pyrido[1,2-a]pyrazine (1.40 g, 2.93 mmol) in CH₃CN (19 mL) at room temperature is treated with morpholine (1.28 mL, 14.7 mmol) followed by K₂CO₃ (0.61 g, 4.40 mmol) and a catalytic amount of KI (0.1 g). The reaction mixture is stirred at 80° C. overnight. After cooling, the reaction mixture is diluted with water (30 mL) and extracted three times with DCM. The combined extracts are dried over Na₂SO₄ and concentrated. The residue is purified by flash chromatography on silica gel. Elution with 1:1 hexanes-EtOAc followed by 100% EtOAc and finally 20:1 CHCl₃—MeOH affords the title product, which is dissolved in EtOAc (˜10 mL) and treated with 1.0 M HCl in Et₂O (2.0 eq). The resulting slurry is stirred at room temperature for 30 min, filtered, and the solid washed with Et₂O and dried to yield the bis-HCl salt. Free base ¹H NMR (CDCl₃, 400 MHz): 7.36 (d, J=8.4 Hz, ˜0.8H), 7.18 (d, J=8.8 Hz, 1H), 6.89 (br, ˜0.2H), 6.74 (d, J=8.8 Hz, ˜0.8H), 6.61 (br, ˜0.2H), 6.46 (d, J=2.8 Hz, 1H), 6.41 (dd, J=8.8, 2.8 Hz, 1H), 4.00 (t, J=6.2 Hz, 2H), 3.87 (s, 3H), 3.73 (t, J=4.6 Hz, 4H), 3.45 (d, J=11.2 Hz, 1H), 3.34 (m, ˜2H), 3.08 (br, ˜0.2H), 2.82-2.72 (m), 2.63 (m), 2.55 (m), 2.48 (br), 2.36 (s, 3H), 2.18 (s, 3H), 1.99 (m), 1.82 (m), 1.70 (m), 1.48 (m) ppm. LC/MS: 528 (M+1).

### Example 12

### N-(3-{4-[(6R,9AS)-2-(4-CHLORO-3-METHOXY-PHENYL)-OCTAHYDRO-PYRIDO[1,2-A]PYRAZIN-6-YL]-2,3-DIMETHYL-PHENOXY}-PROPYL)-ACETAMIDE

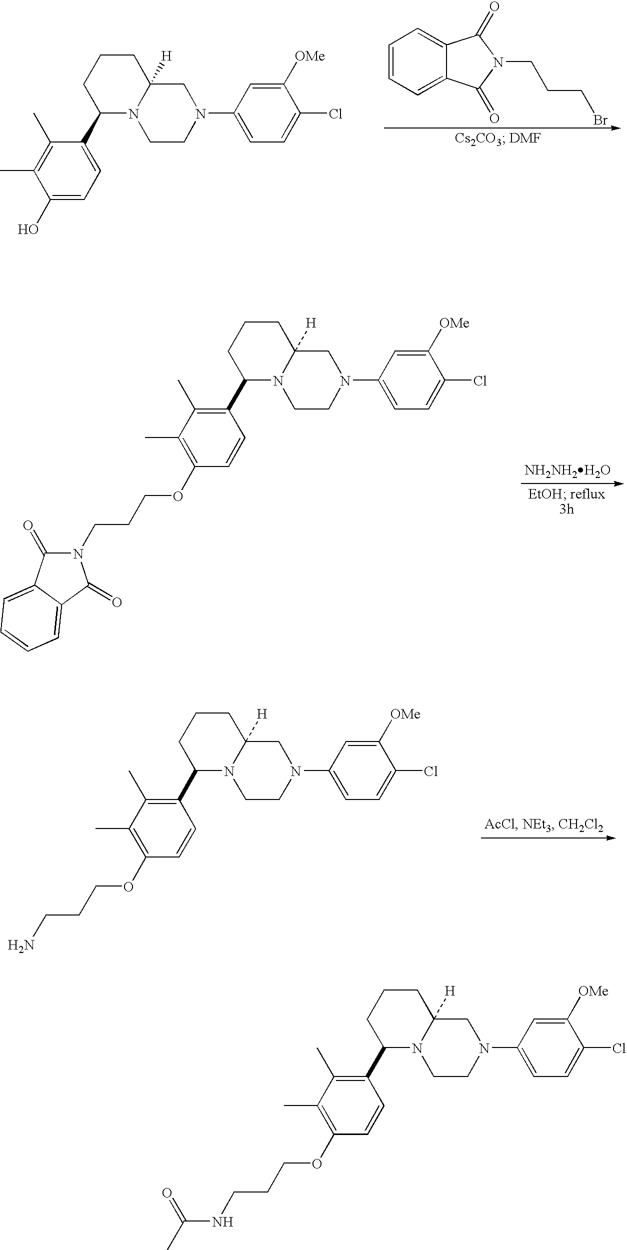

### Step 1. 2-(3-{4-[2-(4-Chloro-3-methoxyphenyl)-octahydro-pyrido[1,2-a]pyrazin-6-yl-2,3-dimethylphenoxy}-propyl)-isoindole-1,3-dione

To a solution of 4-[2-(4-chloro-3-methoxyphenyl)-octahydro-pyrido[1,2-a]pyrazin-6-yl]-2,3-dimethylphenol (obtained as in Example 11, 8 g, 0.026 mol) in anhydrous DMF (100 mL) is added Cs₂CO₃ (7.8 g, 0.024) with stirring. After stirring at room temperature for 30 min, N-(3-bromopropyl)-phthalimide (7 g, 0.026 mol) is added and the stirring is continued for 18 h at room temperature. The reaction mixture is poured into ice-cold water (300 mL) with stirring. The precipitated solid is filtered, washed with water and dried under reduced pressure to afford the title product. ¹H NMR (300 MHz, CDCl₃): 7.81-7.86 (m, 2H), 7.70-7.73 (m, 2H), 7.34 (d, J=6.3 Hz, 1H), 7.17 (d, J=6.3 Hz, 1H), 6.70 (d, J=6.6 Hz, 1H), 6.43 (brs, 1H), 6.41 (dd, J=6.6, 4.8 Hz, 1H), 4.0 (t, J=4.5 Hz, 2H), 3.94 (t, J=5.1 Hz, 2H), 3.90 (s, 3H), 3.40-3.46 (m, 1H), 3.32 (t, J=7.5 Hz, 2H), 2.52-2.81 (m, 4H), 2.32 (t, 7.5 Hz, 1H), 2.14-2.21 (m, 8H), 1.97-2.0 (m, 2H), 1.64-1.72 (m, 2H), 1.41-1.49 (m, 2H); LC/MS: 588 (M+1).

### Step 2. 3-{4-[2-(4-Chloro-3-methoxyphenyl)-octahydro-pyrido[1,2-a]pyrazin-6-yl]-2,3-dimethylphenoxy}-propylamine

2-(3-{4-[2-(4-Chloro-3-methoxyphenyl)-octahydro-pyrido[1,2-a]pyrazin-6-yl]-2,3-dimethyl-phenoxy}-propyl)-isoindole-1,3-dione obtained in step 6 (10 g, 0.017 mol) and N₂H₄.H₂O (150 mL) are dissolved in 300 mL of EtOH and refluxed for 3 h. The reaction mixture is cooled down to room temperature, diluted with 200 mL of DCM, washed with 100 mL of 1N NaOH solution, water, brine, dried over Na₂SO₄, and concentrated under reduced pressure. The residue is purified by silicagel chromatography eluting with 10% MeOH-DCM containing 1% of NH₄OH to afford the title product as a dry foam. ¹H NMR (300 MHz, CDCl₃): 7.37 (d, J=6.3 Hz, 1H), 7.17 (d, J=6.3 Hz, 1H), 6.74 (d, J=6.6 Hz, 1H), 6.46 (brs, 1H), 6.41 (dd, J=1.8, 4.8 Hz, 1H), 4.02 (t, J=4.2 Hz, 2H), 3.86 (s, 3H), 3.45 (d, J=8.7 Hz, 1H), 3.32-3.35 (m, 2H), 2.91-3.01 (m, 2H), 2.60-2.82 (m, 5H), 2.23 (s, 3H), 2.18 (s, 3H), 1.82-2.01 (m, 4H), 1.66-1.72 (m, 2H), 1.42-1.50 (m, 2H); LC-MS found 458 (MH+).

### Step 3. N-(3-{4-[(6R,9aS)-2-(4-chloro-3-methoxy-phenyl)-octahydro-pyrido[1,2-a]pyrazin-6-yl]-2,3-dimethyl-phenoxy}-propyl)-acetamide

To a cooled solution of 3-{4-[2-(4-chloro-3-methoxyphenyl)-octahydro-pyrido[1,2-a]pyrazin-6-yl]-2,3-dimethylphenoxy}-propylamine (6.5 g, 0.014 mol) in dry DCM (80 mL) and NEt₃ (3.9 mL, 0.028 mol) is added acetyl chloride (1 mL, 0.014 mol) dropwise, and the reaction mixture is stirred overnight at room temperature. The volatiles are evaporated under reduced pressure and the organic residue is submitted to flash chromatography over silicagel eluting with 5% MeOH-DCM containing few drops of NH₄OH to afford the title product in 92:8 enantiomeric ratio. This is recrystallized from i-PrOH to obtain the title product in 99% enantiomeric purity; [α]_{D}=+20.9 (c=0.34 g/100 mL, CHCl₃); ¹H NMR (300 MHz, CDCl₃): 7.39 (d, J=6.6 Hz, 1H), 7.18 (d, J=6.6 Hz, 1H), 6.74 (d, J=6.3 Hz, 1H), 6.46 (d, J=1.8 Hz, 1H), 6.40 (dd, J=1.8, 4.5 Hz, 1H), 5.91 (m, 1H), 4.03 (t, J=3.9 Hz, 2H), 3.86 (s, 3H), 3.44-3.51 (m, 3H), 3.32 (d, J=8.4 Hz, 1H), 2.72-2.81 (m, 2H), 2.60-2.65 (m, 2H), 2.35-2.42 (m, 2H), 2.25 (s, 3H), 2.20 (s, 3H), 1.99-2.0 (m, 2H), 1.97 (s, 3H), 1.61-1.82 (m, 4H), 1.45-1.50 (m, 2H). LC/MS: 500 (M+1).

### Example 13

### N-(3-{4-[(6R,9AS)-2-(4-FLUORO-3-METHOXY-PHENYL)-OCTAHYDRO-PYRIDO[1,2-A]PYRAZIN-6-YL]-2,3-DIMETHYL-PHENOXY}-PROPYL)-ACETAMIDE

This compound is prepared using the same protocols outlined in the previous two Examples, starting with 4-(4-fluoro-3-methoxyphenyl)-2-(2-oxo-propyl)-piperazine-1-carboxylic acid tert-butyl ester.

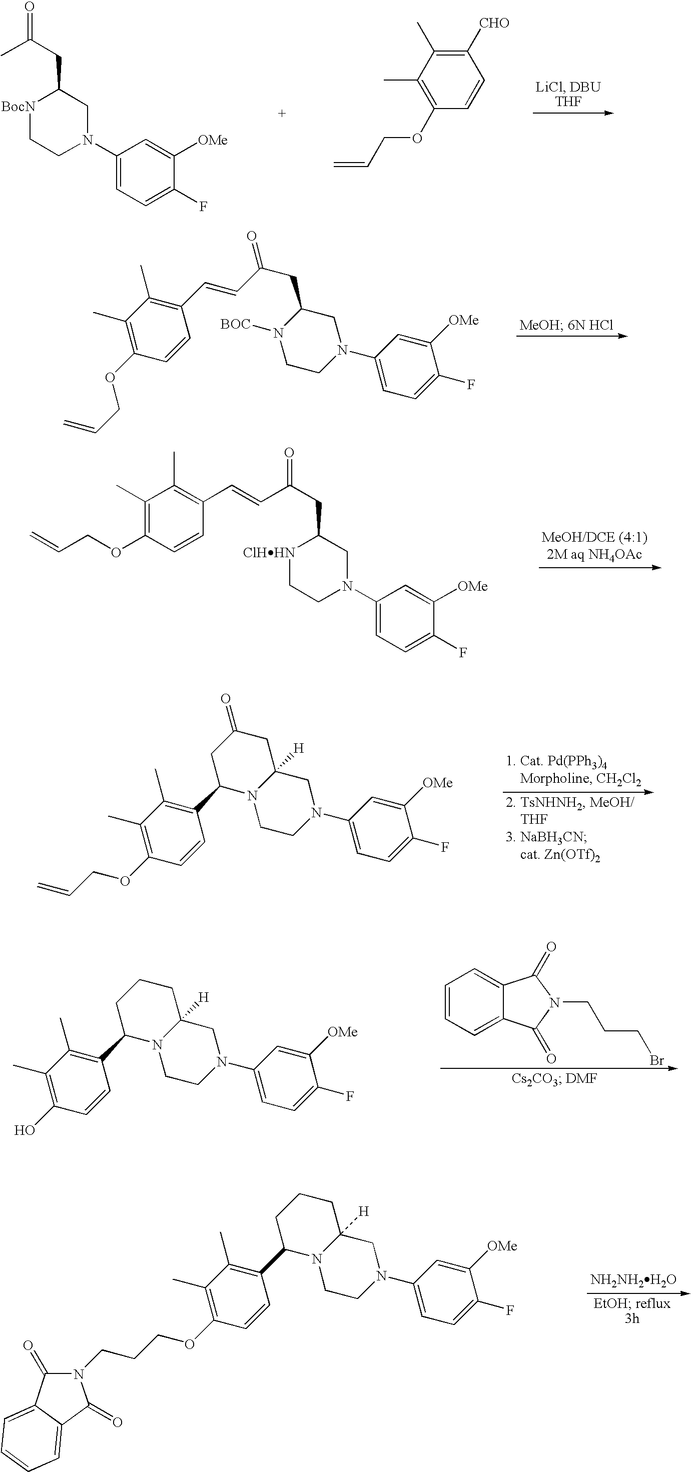

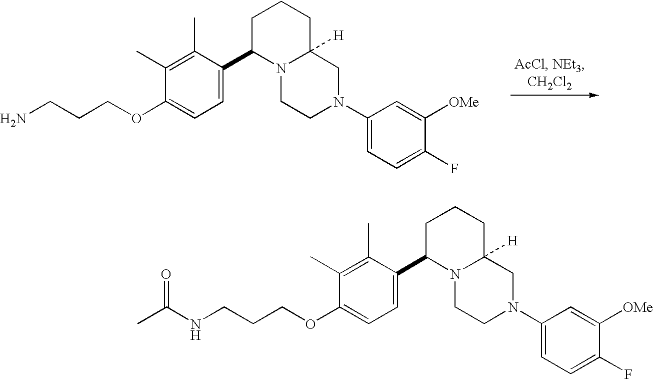

### Step 1. 2-[4-(4-Allyloxy-2,3-dimethylphenyl)-2-oxo-but-3-enyl]-4-(4-fluoro-3-methoxyphenyl)-piperazine-1-carboxylic acid tert-butyl ester

¹H NMR (300 MHz, CDCl₃): 8.02 (br, 1H), 7.42 (d, J=6.6 Hz, 1H), 6.93 (t, J=6.6 Hz, 1H), 6.72 (d, J=6.6 Hz, 1H), 6.63 (d, J=11 Hz, 1H), 6.36-6.39 (m, 1H), 5.99-6.12 (m, 1H), 5.43 (d, J=14 Hz, 1H), 5.28 (d, J=9 Hz, 1H), 4.53 (d, J=3.9 Hz, 1H), 4.01-4.19 (m, 1H), 3.83 (s, 3H), 3.23-3.62 (m, 4H), 2.74-2.89 (m, 2H), 2.40-2.54 (m, 3H), 2.35 (s, 3H), 2.21 (s, 3H), 1.48 (s, 9H); LC/MS: 539 (M+1).

### Step 2. 6-(4-Allyloxy-2,3-dimethyl-phenyl)-2-(4-fluoro-3-methoxy-phenyl)-octahydro-pyrido[1,2-a]pyrazin-8-one

¹H NMR (300 MHz, CDCl₃): 7.41 (br, 1H), 6.93-6.98 (m, 1H), 6.54-6.71 (m, 1H), 6.53 (dd, J=2.1; 3.6 Hz, 1H), 6.38-6.41 (m, 1H), 6.08-6.18 (m, 1H), 5.43 (d, J=14 Hz, 1H), 5.28 (d, J=9 Hz, 1H), 4.53 (d, J=3.9 Hz, 1H), 3.87 (s, 3H), 3.64-3.81 (m, 1H), 3.42 (d, J=6.3 Hz, 1H), 3.33 (d, J=8.7 Hz, 1H), 2.71-2.88 (m, 4H), 2.40-2.54 (m, 3H), 2.24 (s, 6H), 2.05-2.07 (m, 2H). LC/MS: 439 (M+1).

### Step 3. 4-[2-(4-Fluoro-3-methoxyphenyl)-octahydro-pyrido[1,2-a]pyrazin-6-yl]-2,3-dimethyl-phenol.

¹H NMR (300 MHz, CDCl₃): 7.30 (d, J=6.3 Hz, 1H), 6.93 (t, J=6.6 Hz, 1H), 6.64 (d, J=6.3 Hz, 1H), 6.53 (dd, J=2.1; 3.6 Hz, 1H), 6.36-6.40 (m, 1H), 4.01 (br, 2H), 4.74 (br, 1H), 3.86 (s, 3H), 3.25-3.39 (m, 3H), 2.68-2.81 (m, 2H), 2.57-2.62 (m, 4H), 2.34-2.38 (m, 1H), 2.42 (s, 3H), 2.22 (s, 3H), 1.81-2.01 (m, 2H), 1.65-1.74 (m, 2H), 1.45-1.50 (m, 2H). LC/MS: 385 (M+1).

### Step 4. 2-(3-{4-[2-(4-Fluoro-3-methoxyphenyl)-octahydro-pyrido[1,2-a]pyrazin-6-yl]-2,3-dimethylphenoxy}-propyl)-isoindole-1,3-dione

¹H NMR (300 MHz, CDCl₃): 7.81-7.84 (m, 2H), 7.69-7.73 (m, 2H), 7.33 (d, J=8.4 Hz, 1H),), 6.93 (t, J=8.7 Hz, 1H), 6.69 (d, J=8.7 Hz, 1H), 6.52 (d, J=8.7 Hz, 1H), 6.34-6.39 (m, 1H), 4.0 (t, J=6.0 Hz, 2H), 3.91 (t, J=6.9 Hz, 2H), 3.84 (s, 3H), 3.25-3.41 (m, 3H), 2.57-2.81 (m, 4H), 2.36-2.38 (m, 1H), 2.94 (s, 3H), 2.87 (s, 3H), 2.56-2.80 (m, 2H), 1.80-2.01 (m, 2H), 1.64-1.72 (m, 2H), 1.44-1.52 (m, 2H). LC/MS: 572 (M+1).

### Step 5. 3-{4-[2-(4-Fluoro-3-methoxyphenyl)-octahydro-pyrido[1,2-a]pyrazin-6-yl]-2,3-dimethylphenoxy}-propylamine

¹H NMR (300 MHz, CDCl₃): 7.35 (d, J=6.3 Hz, 1H), 6.92 (t, J=6.6 Hz, 1H), 6.73 (d, J=6.6 Hz, 1H), 6.52 (d, J=6.6 Hz, 114), 6.36 (d, J=6.6 Hz, 1H), 4.01 (brs, 2H), 3.84 (s, 3H), 3.23-3.37 (m, 3H), 2.91-3.02 (m, 2H), 2.56-2.80 (m, 4H), 2.35 (t, J=7.5 Hz, 1H), 2.22 (s, 3H), 2.17 (s, 3H), 1.80-2.01 (m, 4H), 1.64-1.72 (m, 2H), 1.44-1.52 (m, 2H). LC/MS: 442 (M+1).

### Step 6 N-(3-(4-[(6R,9aS)-2-(4-fluoro-3-methoxy-phenyl)-octahydro-pyrido[1,2-a]pyrazin-6-yl]-2,3-dimethyl-phenoxy)-propyl)-acetamide

¹H NMR (300 MHz, CDCl₃): 7.38 (d, J=8.7 Hz, 1H), 6.93 (t, J=9.0 Hz, 1H), 6.73 (d, J=8.4 Hz, 1H), 6.53 (d, J=8.4 Hz, 1H), 6.37 (d, J=8.4 Hz, 1H), 5.88 (m, 1H), 4.03 (t, J=5.4 Hz, 2H), 3.86 (s, 3H), 3.48 (q, J=6 Hz, 2H), 3.24-3.39 (m, 3H), 2.56 2.76 (m, 4H), 2.31-2.41 (m, 1H), 2.24 (s, 3H), 2.20 (s, 3H), 2.02 (t, J=6.0 Hz, 2H), 1.97 (s, 3H), 1.64-1.81 (m, 4H), 1.45-1.50 (m, 2H); LC/MS: 484 (M+1).

### Example 14

### 4-(4-CHLORO-3-TRIFLUOROMETHYL-PHENYL)-1-[4-(2-METHOXY-ETHOXY)-2,3-DIMETHYL-BENZYL]-PIPERIDIN-4-OL

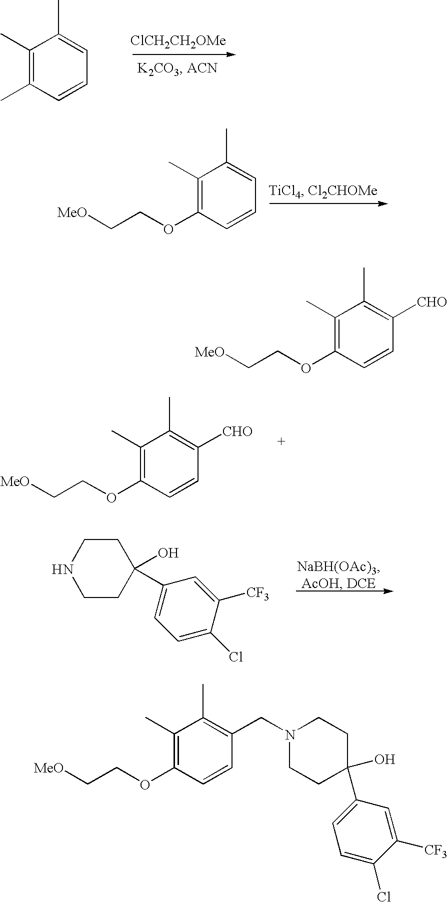

### Step 1. 1-(2-methoxyethoxy)-2,3-dimethylbenzene

To a solution of 2,3-dimethylphenol (57 g, 0.47 mol) and allyl bromide (68 g, 49 mL, 0.56 mol) in acetonitrile (700 mL) is added KOH (37 g, 0.65 mol). The reaction mixture is stirred vigorously at room temperature for 18 h. The solvent is removed under reduced pressure, and the solid residue is partitioned between water and Et₂O. The aqueous layer is washed with Et₂O, the organic layers are combined and washed with brine until neutral pH of the aqueous phase, dried with MgSO₄ and filtered. Removal of solvent under reduced pressure yields the title compound as a dark-colored liquid. ¹H NMR (CDCl₃, 300 MHz): 7.03 (t, 1H); 6.79 (d, 1H); 6.72 (d, 1H); 6.09 (m, 1H), 5.44 (d, 1H); 5.27 (d, 1H); 4.52 (d, 2H); 2.3 (s, 3H); 2.2 (s, 3H).

### Step 2. 4-(2-methoxyethoxy)-2,3-dimethylbenzaldehyde

A solution of TiCl₄ (106.4 g, 62 mL, 0.56 mol) in anhydrous DCM (250 mL) is cooled down to −78° C. (acetone-dry ice bath) under a nitrogen atmosphere (balloon). α,α-Dichloro-methyl methyl ether (Cl₂CHOMe, Aldrich Chemical Co., 35.5 g, 27 mL, 0.31 mol) is added dropwise via syringe maintaining the reaction temperature below −60° C. 1-(2-Methoxy-ethoxy)-2,3-dimethylbenzene (45.5 g, 0.28 mol) is dissolved in anhydrous DCM (250 mL) and added slowly over 1 hour, keeping the reaction mixture at a temperature below −60° C. by continuous addition of dry ice; the reaction mixture turns dark red. Stirring is continued overnight, allowing the reaction mixture to slowly reach room temperature. The reaction is quenched by pouring it into a large flask containing crushed ice (500 g) and concentrated HCl (50 mL) with vigorous stirring. After 30 min, the 2 phases are separated, the organic phase washed with NaHCO₃ (5% in water) several times (until neutral pH of the aqueous phase is obtained) and then once with brine. The organic phase is dried and flashed through a 10-cm plug of silicagel, eluting with EtOAc, to remove inorganic impurities and part of the dark color. Upon evaporation of the solvent, the title compound is obtained as an off-white solid. ¹H NMR (CDCl₃, 300 MHz): 10.3 (s, 1H); 7.62 (d, 1H); 6.81 (d, 1H); 6.07 (m, 1H), 5.44 (d, 1H); 5.32 (d, 1H); 4.62 (d, 2H); 2.6 (s, 3H); 2.2 (s, 3H).

### Step 3. 4-(4-Chloro-3-trifluoromethyl-phenyl)-1-[4-(2-methoxy-ethoxy)-2,3-dimethyl-benzyl]-piperidin-4-ol

To a solution of 4-(2-methoxyethoxy)-2,3-dimethylbenzaldehyde (0.5 g, 2 mmol) and 4-[4-chloro-3(trifluoromethyl)phenyl]-4-piperidinol (0.57 g, 2 mmol) in anhydrous CH₂ClCH₂Cl (10 mL) is added NaBH(OAc)₃ (1.5 g, 3 mmol) and catalytic amounts (0.1 mL) of AcOH. The mixture is stirred overnight at room temperature. The reaction mixture is taken to dryness under reduced pressure (rotary evaporator) and the organic residue is diluted with 100 mL of EtOAc. The organic layer is washed with saturated aqueous NaHCO₃, brine, dried over Na₂SO₄ and concentrated to an oil under reduced pressure. The residue is purified by silica gel chromatography eluting with 10% MeOH-DCM containing 1% of NH₄OH to afford the title compound as a dry foam. ¹H NMR (300 MHz, CDCl₃): 7.84 (s, 1H), 7.59 (d, J=6.6 Hz, 1H), 7.45 (d, J=6.6 Hz, 1H), 7.07 (d, J=6.6 Hz, 1H), 6.67 (d, J=6.0 Hz, 1H), 4.09 (t, J=3.6 Hz, 2H), 3.76 (t, J=3.6 Hz, 2H), 3.58 (s, 2H), 3.46 (s, 3H), 2.87 (d, J=8.4 Hz, 2H), 2.51 (t, J=8.4 Hz, 2H), 2.30 (s, 3H), 2.20 (s, 3H), 2.04-2.14 (m, 2H), 1.69 (d, J=9 Hz, 2H). LC/MS: 472 (M+1).

### Example 15

### SYNTHESIS OF RACEMIC [(6R,9AS)-6-(4-METHOXY-2,3-DIMETHYLPHENYL)-OCTAHYDRO-PYRIDO[1,2-A]PYRAZIN-2-YL]-(6-TRIFLUOROMETHYL-PYRIDIN-3-YL)-METHANONE

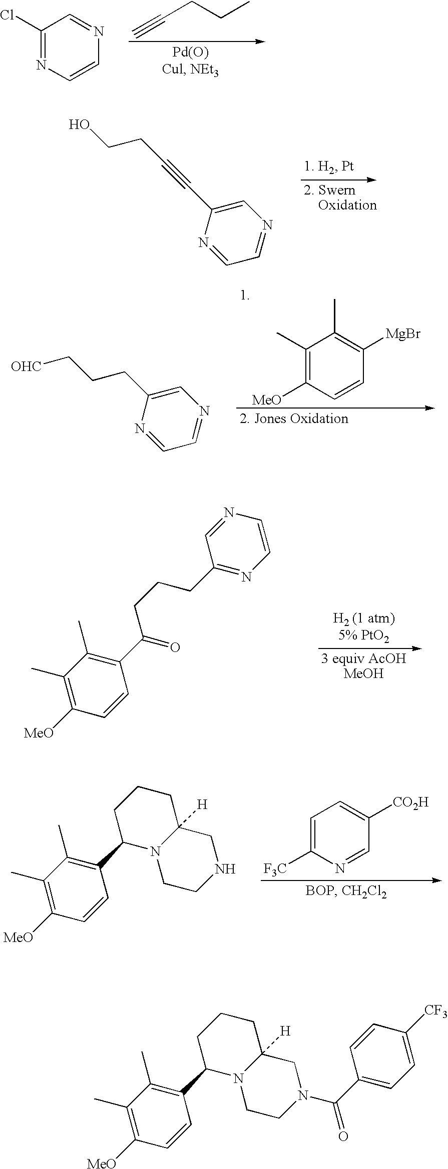

### Step 1. 4-Pyrazin-2-yl-but-3-yn-]-ol

A mixture of 2-chloropyrazine (65 g, 0.57 mol), 3-butyn-1-ol (51.8 g, 0.74 mol), PdCl₂(PPh₃)₂ (7 g, 10 mmol), CuI (1.9 g, 10 mmol) and NEt₃ (500 mL) is stirred in a pressure tube at 50° C. (oil bath temperature) for 4 h and then at room temperature for 16 h. The reaction mixture is filtered through a thick celite plug (5 cm), and washed with NEt₃ (100 mL) and EtOAc (500 mL). The solvents are eliminated under reduced pressure (rotavapor). The resulting black residue is taken in DCM and filtered through a silicagel column (10 cm), eluting with EtOAc, which removes black impurities and yields the title product as a cream-colored solid. ¹H NMR (CDCl₃, 400 MHz): 8.62 (s, 1H); 8.50 (s, 1H); 8.44 (s, 1H); 3.88 (t, 2H); 2.89 (t, 2H); 2.4 (br, 1H).

### Step 2. 4-Pyrazin-2-yl-butan-1-ol

4-Pyrazin-2-yl-but-3-yn-1-ol (5.4 g, 36.5 mmol) is dissolved in EtOAc (300 mL) and EtOH (300 mL). Pd catalyst is added (0.7 g, 10%/C). The reaction mixture is degassed for 5 min under vacuum and then H₂ (balloon) is added. After 2 h an additional 0.15 g of catalyst is added and the hydrogenation reaction mixture is stirred overnight at room temperature. The flask is evacuated and purged with nitrogen. The reaction mixture is filtered through a celite plug to remove the heterogeneous catalyst. The solvent is evaporated under reduced pressure (rotavapor) and the oily residue is purified by flash chromatography, eluting with EtOAc. 4-Pyrazin-2-yl-butan-1-ol is obtained as a yellow oil upon evaporation of the solvent. ¹H NMR (CDCl₃, 300 MHz): 8.43 (s, 2H); 3.67 (t, 2H); 2.84 (m, 2H); 1.97 (br, 1H); 1.82 (m, 2H); 1.62 (m, 2H). LC/MS: 135 (M+1).

### Step 3. 4-Pyrazin-2-yl-butyraldehyde

A solution of oxalyl chloride (6.6 mL, 2M in DCM, 13.2 mmol) is cooled to −42° C. (acetonitrile/dry ice bath). To this solution is added anhydrous DMSO (1.87 mL, 26.4 mmol) and the mixture is stirred for 20 min at the same temperature. A solution of 4-pyrazin-2-yl-butan-1-ol (1.0 g, 6.6 mmol) in anhydrous DCM (40 mL) is added and the reaction mixture is stirred at 42° C. for 1 h. NEt₃ (7.4 mL, 52.8 mmol) is added. Stirring is continued at that temperature for 30 min and then at room temperature for 2 h. The reaction is quenched by diluting with DCM. The resulting solution is washed with brine and dried over Na₂SO₄. Upon concentration in vacuo a dark-colored oil is obtained, which is filtered through a silicagel plug eluting with EtOAc/hexanes 1:1 to furnish the title product as a light brown oil. ¹H NMR (CDCl₃, 400 MHz): 9.78 (s, 114); 8.50 (s, 1H); 8.46 (s, 1H); 8.42 (s, 1H); 2.86 (t, 2H); 2.54 (t, 2H); 2.10 (m, 2H).

### Step 4. 1-(4-Methoxy-2,3-dimethyl-phenyl)-4-pyrazin-2-yl-butan-1-ol

All glassware used in this reaction is oven-dried and cooled under a nitrogen stream. 4-Bromo-2,3-dimethylanisole (1.0 g, 4.65 mmol) is dissolved in anhydrous THF (10 mL). An aliquot of this solution (2 mL) is added to Mg turnings (226 mg, 9.3 mmol) in anhydrous THF (10 mL). (The Mg turnings were previously placed in the flask and heated with heat-gun for 5 min.) A small crystal of I₂ is added to start the formation of the organomagnesium reagent, and the mixture is heated with heat-gun to reflux temperature. The purple color of the solution disappears in 5 min. The rest of the solution of the aryl bromide in THF is then added in one portion and the reaction mixture is heated at reflux temperature for 4 h to complete the generation of the Grignard reagent. Upon cooling to −78° C., a solution of 4-pyrazin-2-yl-butyraldehyde (1.9 mmol, 285 mg) in THF (10 mL) is added dropwise. The resulting mixture is stirred at −78° C. for 1 h and then at room temperature for 1 h. The reaction is quenched by addition of brine. The mixture is partitioned with EtOAc (2×), the organic layers combined and washed with brine, dried with Na₂SO₄ and concentrated under reduced pressure. The residue is purified by filtration through a silicagel plug to yield the title product as a yellow-brown oil. ¹H NMR (CDCl₃, 400 MHz): 8.46 (s, 1H); 8.44 (s, 1H); 8.38 (s, 1H); 7.27 (d, 1H); 6.74 (d, 1H); 4.97 (t, 1H); 3.81 (s, 3H); 2.86 (t, 2H); 2.16 (s, 3H); 2.04 (s, 3H); 1.7-2.1 (m, 4H). LC/MS: 269 (M−H₂O+1).

### Step 5. 1-(4-Methoxy-2,3-dimethyl-phenyl)-4-pyrazin-2-yl-butan-1-one

A solution of 1-(4-methoxy-2,3-dimethyl-phenyl)-4-pyrazin-2-yl-butan-1-ol (50 mg, 0.17 mmol) in anhydrous DCM (2 mL) is added to a solution of Dess-Martin periodinane (168 mg, 0.26 mmol) in anhydrous DCM (3 mL). The resulting mixture is stirred for 45 min at room temperature. The reaction is quenched by addition of EtOAc (10 mL) and NaOH (1N, 5 mL) and stirred at room temperature for 10 min. The organic layer is partitioned with 1N NaOH and brine, dried over Na₂SO₄. Evaporation of the solvent under reduced pressure yields the title product as a brown oil. ¹H NMR (CDCl₃, 400 MHz): 8.49 (s, 2H); 8.41 (s, 1H); 7.44 (d, If); 6.70 (d, 1H); 3.85 (s, 3H); 2.92 (m, 4H); 2.37 (s, 3H); 2.19 (m, 2H); 2.17 (s, 3H). LC/MS: 285 (M+1).

### Step 6. Racemic (6,9a)-6-(4-methoxy-2,3-dimethylphenyl)-octahydro-pyrido[1,2-a]pyrazine

A solution of 1-(4-methoxy-2,3-dimethyl-phenyl)-4-pyrazin-2-yl-butan-1-one (44.8 mg, 0.16 mmol) in MeOH (4 mL) containing acetic acid (0.47 mmol) and PtO₂ (10 mg) is shaken under an atmosphere of H₂ (balloon) for 24 h. The reaction mixture is then filtered through a pad of celite using MeOH. The filtrate is concentrated in vacuo. The residue is triturated with acetone and filtered. The solid is washed with acetone and dried yielding the title product dihydrochloride as a tan solid. A small sample is free-based (1N NaOH/DCM) and the resulting oil used to record the ¹H NMR spectrum. ¹H NMR (400 MHz, CDCl₃): 7.36 (d, J=8.8 Hz, 0.9H), 6.88 (br, 0.1H), 6.73 (d, J=8.8 Hz, 0.9H), 6.61 (br, 0.1H), 3.79 (s, 3H), 3.28 (d, J=7.6 Hz, 0.9H), 3.02 (br, 0.1H), 2.88-2.55 (m, 6H), 2.22 (s, 3H), 2.16 (s, 3H), 2.18-1.31 (m, 8H). LC/MS: 274 (M+1).

### Step 7. [6-(4-Methoxy-2,3-dimethyl-phenyl)-octahydro-pyrido[1,2-a]pyrazin-2-yl]-(6-trifluoro-methyl-pyridin-3-yl)-methanone

A solution of cis-6-(4-methoxy-2,3-dimethylphenyl)-octahydropyrido[1,2-a]pyrazine (5.5 mg, 0.02 mmol), 6-trifluoromethylnicotinic acid (4.2 mg, 0.022 mmol) and BOP (13.3 mg, 0.03 mmol) in 5% NEt₃ in DMA (0.5 mL) is stirred at room temperature for 16 h. The reaction mixture is diluted with EtOAc and washed with NaOH 1N (2×10 mL) and brine (2×10 mL), dried (MgSO₄), filtered and concentrated under reduced pressure. The residue is purified by PTLC on silicagel, eluting with 5% MeOH and 1% NH₃ in DCM to yield the title compound as an oil. LC/MS: 448 (M+1).

### Example 16

### SYNTHESIS OF RACEMIC (6,9A)-6-(4-METHOXY-2,3-DIMETHYLPHENYL)-OCTAHYDRO-PYRIDO[1,2-A]PYRAZINE THROUGH HYDROBORATION/PD(0)—COUPLING REACTIONS

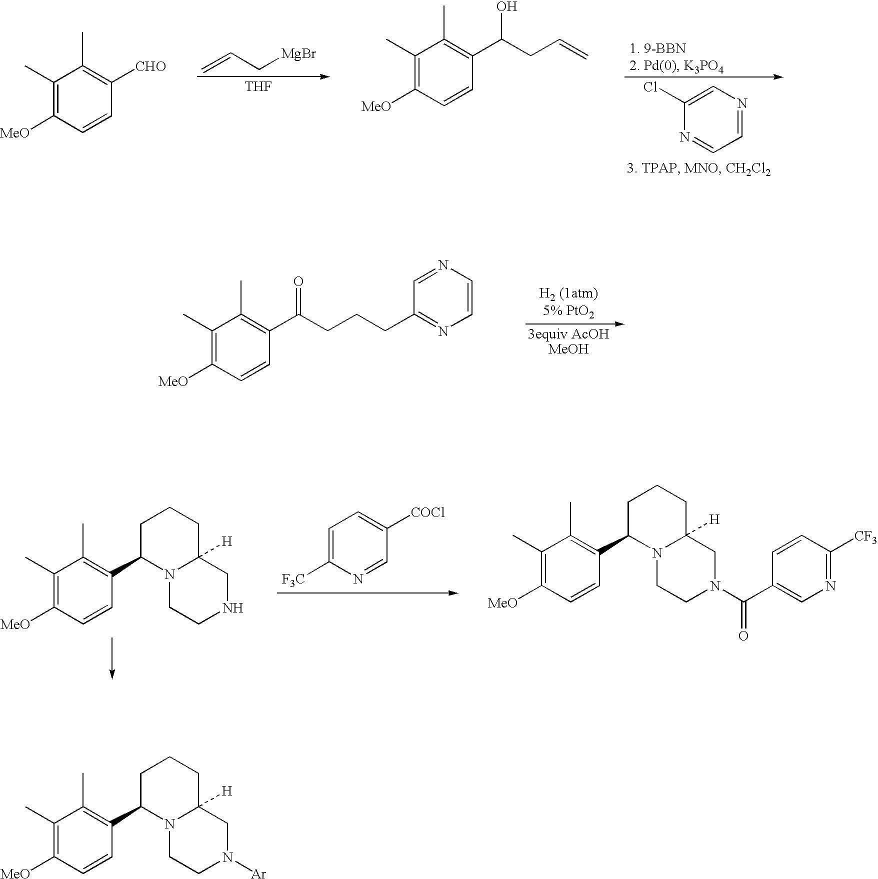

### Step 1. 1-(4-Methoxy-2,3-dimethylphenyl)-but-3-en-1-ol

2,3-Dimethyl-4-methoxybenzaldehyde (328 mg, 2.0 mmol) is dissolved in anhydrous THF (16 mL) at −78° C. under a nitrogen atmosphere. Allylmagnesium chloride (2.0M in THF, 1.3 mL, 2.6 mmol) is added dropwise over 2 min. The reaction mixture is kept at −78° C. for 1 h and then allowed to reach room temperature. An additional amount of the Grignard reagent is added (0.3 mL) and the reaction is stirred for an additional hour at room temperature. The reaction is quenched by addition of H₂O (1 mL) at 0° C. and then NH₄Cl (saturated solution). The crude product is isolated by partition between Et₂O and brine. PTLC on silicagel eluting with 25% EtOAc in hexanes yields the title product as a clear oil. ¹H NMR (CDCl₃, 400 MHz): 7.30 (d, 1H); 6.76 (d, 1H); 5.87 (m, 1H); 5.28 (m, 1H); 5.16 (m, 1H); 4.98 (m, 1H); 3.80 (s, 3H); 2.40-2.55 (m, 2H); 2.25 (s, 3H); 2.18 (s, 3H). LC/MS: 189 (M−H₂O+1).

### Step 2. 1-(4-Methoxy-2,3-dimethylphenyl)-4-pyrazin-2-yl-butan-1-ol

9-BBN (solid dimmer, 146 mg, 1.2 mmol) is weighed out into a flame-dried flask. Anhydrous THF (10 mL) is added under a nitrogen atmosphere. A solution of 1-(4-methoxy-2,3-dimethylphenyl)-but-3-en-1-ol (103 mg, 0.5 mmol) in anhydrous THF (1 mL) is added via syringe. The reaction mixture is heated at for 1 h at room temperature and then at 50° C. for 3 h. The reaction mixture is taken to room temperature and treated with K₃PO₄ (1M in H₂O, 1.5 mL), chloropyrazine (0.054 mL, 0.6 mmol) and Pd(PPh₃)₄ (17.3 mg, 3 mol %) and heated for 16 h at 80° C. The reaction mixture is cooled to 0° C. (ice-water bath) and treated with NaOH (0.5 mL, 2.5M) and H₂O₂ (30% in H₂O, 0.2 mL), stirring for 30 min at room temperature. The mixture is partitioned between Et₂O and H₂O, the organic layer is dried over Na₂SO₄ and concentrated under reduced pressure. Chromatography on silicagel eluting with 75% EtOAc in hexanes yields the title product as a clear oil. ¹H NMR (CDCl₃, 400 MHz): 8.46 (s, 1H); 8.44 (s, 1H); 8.38 (s, 1H); 7.27 (d, 1H); 6.74 (d, 1H); 4.97 (t, 1H); 3.81 (s, 3H); 2.86 (t, 2H); 2.16 (s, 3H); 2.04 (s, 3H); 1.7-2.1 (m, 4H). LC/MS: 269 (M−H₂O+1).

### Step 3. 1-(4-Methoxy-2,3-dimethylphenyl)-4-pyrazin-2-yl-butan-1-one

A solution of 1-(4-methoxy-2,3-dimethyl-phenyl)-4-pyrazin-2-yl-butan-1-ol (50 mg, 0.17 mmol) in anhydrous DCM (2 mL) is added to a solution of Dess-Martin periodinane (168 mg, 0.26 mmol) in anhydrous DCM (3 mL). The resulting mixture is stirred for 45 min at room temperature. The reaction is quenched by addition of EtOAc (10 mL) and NaOH (1N, 5 mL) and stirred at room temperature for 10 min. The organic layer is partitioned with 1N NaOH and brine, dried over Na₂SO₄. Evaporation of the solvent under reduced pressure yields the title product as a brown oil. ¹H NMR (CDCl₃, 400 MHz): 8.49 (s, 2H); 8.41 (s, 1H); 7.44 (d, 1H); 6.70 (d, 1H); 3.85 (s, 3H); 2.92 (m, 4H); 2.37 (s, 3H); 2.19 (m, 2H); 2.17 (s, 3H). LC/MS: 285 (M+1).

Racemic 1-(4-methoxy-2,3-dimethyl-phenyl)-4-pyrazin-2-yl-butan-1-one is transformed into [6-(4-methoxy-2,3-dimethyl-phenyl)-octahydro-pyrido[1,2-a]pyrazin-2-yl]-(6-trifluoro-methyl-pyridin-3-yl)-methanone as described in the previous Example.

### Example 17

### SYNTHESIS OF ((1S,4S)-5-((S)-1-(4-((S)-3-HYDROXYBUTOXY)-2,3-DIMETHYLPHENYL)ETHYL)-2,5-DIAZA-BICYCLO[2.2.1]HEPTAN-2-YL)(5-(TRIFLUOROMETHYL)PYRIDIN-2-YL)METHANONE

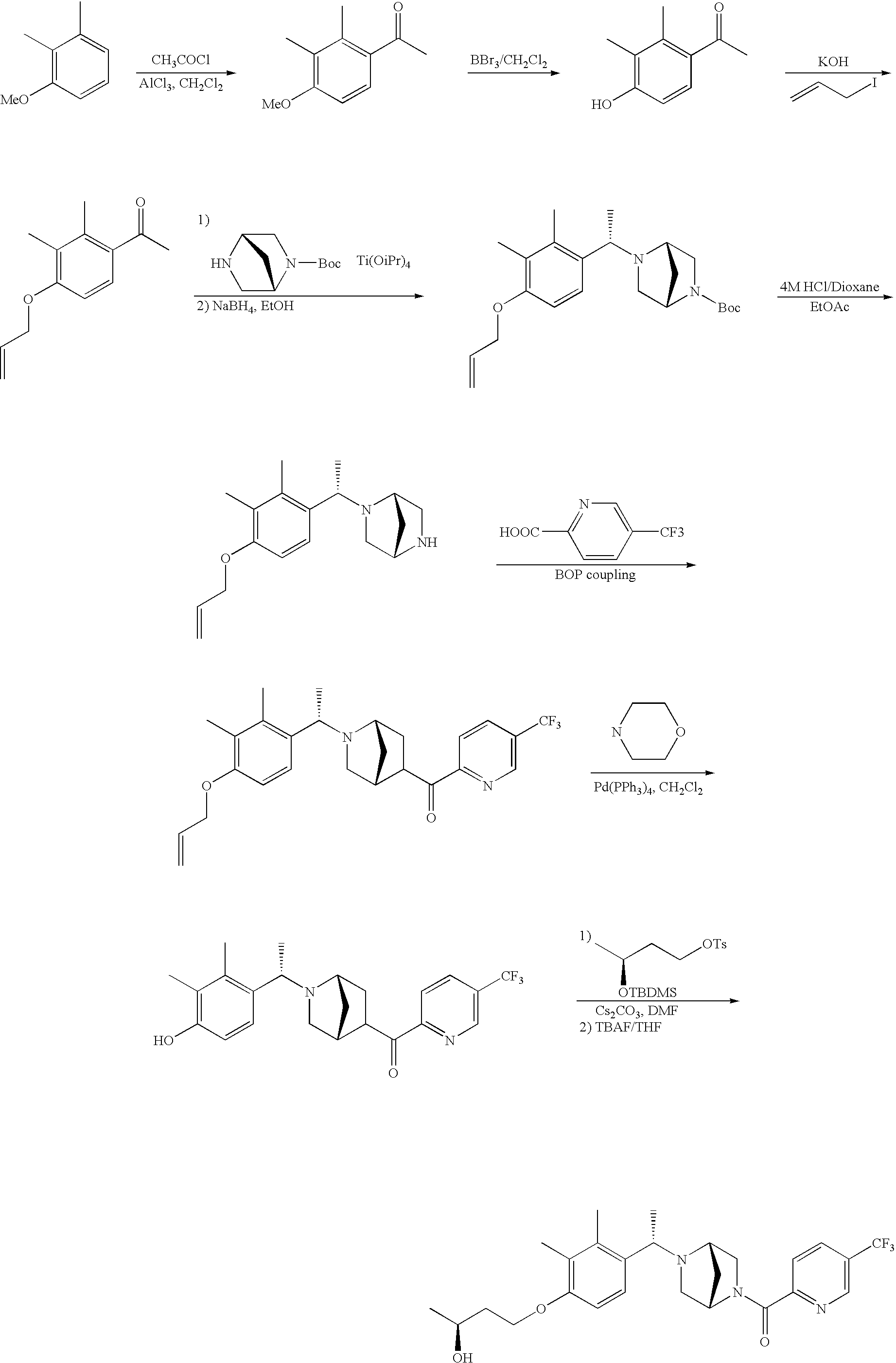

### Step 1. 1-(4-Methoxy-2,3-dimethylphenyl)ethanone

To a solution of aluminum chloride (70.4 g, 0.528 mol) in anhydrous CH₂Cl₂ (400 mL) under N₂ at 0° C. is added acetyl chloride (31.3 mL, 0.44 mol) slowly via an addition funnel, followed by 2,3-dimethylanisole (60 g, 0.44 mol). After stirring for 30 min at 0° C., the reaction mixture is poured onto 600 g of ice cubes and vigorously stirred as conc. HCl (300 mL) is added slowly. After 1 h stirring, the organic layer is isolated, washed with brine, and dried over Na₂SO₄. Removal of the solvent under reduced pressure affords the title compound as an off white oil which becomes white crystalline after stored in refrigerator overnight. ¹H-NMR (300 MHz, CDCl₃) δ: 7.55 (d, 1H), 6.72 (d, 1H), 3.88 (s, 3H), 2.56 (s, 3H), 2.42 (s, 3H), 2.19 (s, 3H). LC-MS m/z (M+H): 179.

### Step 2. 1-(4-Hydroxy-2,3-dimethylphenyl)ethanone

To a solution of 1-(4-methoxy-2,3-dimethylphenyl)ethanone (37 g, 0.21 mol) in anhydrous CH₂Cl₂ (400 mL) under N₂ at −78° C. is added BBr₃ (49.2 mL, 0.52 mol) dropwise via an addition funnel over a period of 45 min while maintaining the internal temperature below −70° C. The reaction mixture is gradually warmed to room temperature and stirred overnight. The reaction mixture is poured carefully into saturated NaHCO₃ solution (1500 mL) containing ice over 30 min with vigorous stirring, and warmed to room temperature gradually. The pH of the aqueous layer is about 6-7. The light pink solid is collected via filtration and washed with water. The solid is redissolved in EtOAc (500 mL), washed with water and brine, and dried over Na₂SO₄. Removal of the solvent under reduced pressure affords the title compound as a light pink solid. The organic layer from the filtration of the solid is separated, and the aqueous phase is extracted with CH₂Cl₂ (2×100 mL). The organic layers are combined, washed with water (2×250 mL), brine (250 mL), dried over Na₂SO₄, and concentrated under reduced pressure. The residue is triturated with CH₂Cl₂/Et₂O (1:1, 50 mL) to afford additional title compound as a light pink solid. ¹H-NMR (400 MHz, DMSO-d₆) δ: 9.96 (s, 1H), 7.49 (d, 1H), 6.70 (d, 1H), 2.43 (s, 3H), 2.29 (s, 3H), 2.05 (s, 3H). LC-MS m/z (M+H): 164.

### Step 3. 1-[4-(Allyloxy)-2,3-dimethylphenyl]ethanone

To a solution of 1-(4-hydroxy-2,3-dimethylphenyl)ethanone (26.26 g, 0.161 mol) in anhydrous acetonitrile (300 mL) under N₂ at room temperature is added powdered KOH (9.92 g, 0.177 mol). After stirring for 10 min, allyl iodide (19.1 mL, 0.209 mol) is added, and the reaction mixture is stirred at room temperature overnight. Acetonitrile is removed under reduced pressure. The residue is diluted with EtOAc, washed with water and brine, dried over Na₂SO₄, and concentrated under reduced pressure. The residue is purified by silica gel chromatography (hexane/EtOAc 95:5) to afford the title compound as a yellow oil. ¹H-NMR (300 MHz, CDCl₃) δ: 7.51 (d, 1H), 6.70 (d, 1H), 6.07 (m, 1H), 5.44 (m, 1H), 5.30 (m, 1H), 4.57-4.59 (m, 2H), 2.54 (s, 3H), 2.43 (s, 3H), 2.21 (s, 3H). LC-MS m/z (M+H): 205.

### Step 4. tert-Butyl (1S, 4S)-5-{(1S)-1-[4-(allyloxy)-2,3-dimethylphenyl]ethyl}-2,5-diazabicyclo[2.2.1]heptane-2-carboxylate

A mixture of 1-[4-(allyloxy)-2,3-dimethylphenyl]ethanone (24.2 g, 0.119 mol) and (1S,4S)-tert-butyl 2,5-diaza-bicyclo[2.2.1]heptane-2-carboxylate (22.8 g, 0.115 mol) in Ti(OiPr)₄ (65.4 g, 0.23 mol) under N₂ is heated at 70° C. for 3 h. The reaction mixture is cooled to 0° C., and anhydrous EtOH (500 mL) is added, followed by NaBH₄ (6.53 g, 0.173 mol) in small portions. The mixture is stirred at 0° C. for 0.5 h. The reaction is quenched by addition of aqueous NaOH (1N, 500 mL), and stirred at room temperature for 0.5 h. Insoluble materials are removed by filtration through celite, and the filter cake is washed with EtOAc. The filtrate and wash are combined and concentrated under reduced pressure. The residue is partitioned between water and EtOAc, organic layer is washed with water and brine, dried over Na₂SO₄, and concentrated under reduced pressure to afford the mixture of two diastereoisomers (ratio 2/1). The residue is purified by silica gel chromatography (hexane/EtOAc: 95/5) to afford the undesired “R” diastereoisomer, mixture of “R” and “S” (S/R=3/1), and the desired “S” diastereoisomer as a brown oil. ¹H-NMR (400 MHz, CDCl₃ δ: 7.25 (d, 1H), 6.70 (d, 1H), 6.07 (m, 1H), 5.43 (m, 1H), 5.26 (m, 1H), 4.51 (d, 2H), 4.34 (bs, 0.5H), 4.25 (bs, 0.5H), 3.80 (m, 1H), 3.32-3.44 (m, 2H), 3.10 (m, 1H), 2.95 (m, 1H), 2.55 (m, 1H), 2.31 (s, 3H), 2.20 (s, 3H), 1.83 (m, 1H), 1.61 (m, 1H), 1.47 (s, 9H), 1.24-1.28 (m, 3H). LC-MS m/z (M+Na): 409.

### Step 5. (1s, 4s)-2-{(1s)-1-[4-(allyloxy)-2,3-dimethylphenyl]ethyl}-2,5-diazabicyclo[2.2.1]heptane

tert-Butyl (1S, 4S)-5-{(1s)-1-[4-(allyloxy)-2,3-dimethylphenyl]ethyl}-2,5-diazabicyclo[2.2.1]-heptane-2-carboxylate (10.0 g, 30 mmol) is dissolved in EtOAc (60 mL) and treated with 4 M HCl in dioxane (60 mL) at room temperature for 4 h. The reaction mixture is then triturated with hexane, and resulting yellow solid is collected via filtration and washed with hexane. The solid is then partitioned between 1N NaOH and EtOAc, the organic layer is washed with brine, dried over Na₂SO₄. Removal of the solvent under reduced pressure affords the title compound as a brown oil. ¹H-NMR (400 MHz, CDCl₃ δ: 7.27 (d, 1H), 6.70 (d, 1H), 6.07 (m, 1H), 5.43 (m, 1H), 5.25 (m, 1H), 4.50 (d, 2H), 3.81 (q, 1H), 3.52 (s, 1H), 3.30 (s, 1H), 3.07-3.13 (m, 2H), 2.63 (dd, 1H), 2.35 (d, 1H), 2.29 (s, 3H), 2.19 (s, 3H), 2.04 (bs, 1H), 1.82 (d, 1H), 1.47 (d, 1H), 1.26 (d, 3H). LC-MS m/z (M+H): 287.

### Step 6. 5-(Trifluoromethyl)pyridine-2-carboxylic acid

To a solution of 2-chloro-5-(trifluoromethyl)pyridine (31.2 g, 0.172 mol) in anhydrous DMF (200 mL) is added zinc cyanide (80.65 g, 0.686 mol). The suspension is stirred at room temperature for 10 min while N₂ is bubbled through. Pd(PPh₃)₄ (9.92 g, 8.6 mmol) is then added, and the reaction mixture is heated at 90° C. under N₂ overnight. The reaction is cooled to room temperature, diluted with 1 N NaOH (2 L), and extracted with EtOAc (2×500 mL). The organic layer is washed with water (3×500 mL), brine (500 mL), dried over Na₂SO₄, and concentrated to about 100 mL under reduced pressure. The concentrated EtOAc solution is filtered through a silica gel plug (250 g), and eluted with EtOAc/hexane (4:1, 1 L) to remove baseline impurities. The filtrate is concentrated under reduced pressure. The residue is then treated with 6 N HCl (50 mL) at 100° C. overnight. The reaction is cooled to 0° C. and the pH is adjusted to 5-6 with 10 N NaOH. The yellow solid is collected via filtration, washed with Et₂O (2×100 mL) and CH₂Cl₂ (2×50 mL) to remove impurities carrying over from the first step. The resulting off-white solid is dried by co-evaporation with toluene to afford the title compound. ¹HNMR (400 MHz, CDCl₃) δ: 8.93 (s, 1H), 8.39 (d, 1H), 8.23 (d, 1H).

### Step 7. (1S, 4S)-2-{(1S)-1-[4-(allyloxy)-2,3-dimethylphenyl]ethyl}-5-[[5-(trifluromethyl)pyridin-2-yl]carbonyl-2,5-diazabicyclo[2.2.1]heptane

A mixture of (1S, 4S)-2-{(15)-1-[4-(allyloxy)-2,3-dimethylphenyl]ethyl}-2,5-diazabicyclo-[2.2.1]heptane (7.3 g, 25.5 mmol), 5-(trifluoromethyl)pyridine-2-carboxylic acid (4.97 g, 26.0 mmol), BOP (16.9 g, 38.3 mmol) and NEt₃ (8.89 mL, 63.8 mmol) in N,N-dimethylacetamide (50 mL) is heated at 40° C. under N₂ overnight. The reaction mixture is cooled to room temperature, diluted with 1N NaOH (100 mL), and extracted with EtOAc. The organic layer is isolated, washed with 1N NaOH, water and brine, dried over Na₂SO₄, and concentrated under reduced pressure. The residue is purified by silica gel chromatography with EtOAc as the eluent to afford the title compound as a brown oil. ¹HNMR (400 MHz, CDCl₃) δ: 8.86 (s, 0.67H), 8.83 (s, 0.33H), 8.02-8.13 (m, 2H), 7.28 (m, 1H), 6.72 (m, 1H), 6.07 (m, 1H), 5.43 (m, 1H), 5.26 (m, 1H), 4.97 (s, 1H), 4.49-4.53 (m, 2H), 3.72-4.00 (m, 2H), 3.52 (m, 1H), 3.35 (m, 1H), 3.22 (m, 1H), 2.83 (d, 0.67H), 2.78 (d, 0.33H), 2.33 (s, 2H), 2.25 (s, 1H), 2.21 (s, 2H), 2.19 (s, 1H), 1.95 (m, 1H), 1.72 (m, 1H), 1.25-1.31 (m, 31). LC-MS m/z (M+H): 460.

### Step 8. 2,3-Dimethylphenyl-4-[(1S)-1-((1S, 4S)-5-{[5-(trifluromethyl)pyridin-2-yl]carbonyl}-2,5-diazabicyclo[2.2.1]hept-2-yl)ethyl]phenol

To a solution of (1S,4S)-2-{(1S)-1-[4-(allyloxy)-2,3-dimethylphenyl]ethyl}-5-{[5-(trifluromethyl)-pyridin-2-yl]carbonyl}-2,5-diazabicyclo[2.2.1]heptane (from step 7, 9.2 g, 20.0 mmol) in anhydrous CH₂Cl₂ (200 mL) is added morpholine (1.92 mL, 22.0 mmol). The solution is purged with nitrogen for 10 min, Pd(PPh₃)₄ (1.16 g, 1.0 mmol) is then added, and the reaction mixture is stirred under nitrogen for 2 h. The solvent is removed under reduced pressure. The residue is diluted with EtOAc (30 mL), insoluble bright yellow catalyst is removed by filtration, and washed with EtOAc (2×30 mL). The filtrate and washings are combined, washed with 1:1 water-saturated NaHCO₃ and brine, dried over Na₂SO₄, and concentrated under reduced pressure. The residue is purified by silica gel chromatography (eluted first with 800 mL of EtOAc, then EtOAc/MeOH (95/5)) to afford the title compound as a yellow solid. ¹HNMR (400 MHz, CDCl₃) δ: 8.87 (s, 0.67H), 8.83 (s, 0.33H), 8.04-8.13 (m, 2H), 7.19 (m 1H), 6.65 (m, 1H), 5.22 (bs, 0.67H), 5.10 (bs, 0.33H), 4.97 (s, 1H), 3.73-3.98 (m, 2H), 3.53 (m, 1H), 3.36 (m, 1H), 3.22 (dd, 0.67H), 3.17 (dd, 0.33H), 2.81 (d, 0.67H), 2.76 (d, 0.33H), 2.32 (s, 2H), 2.24 (s, 1H), 2.18 (s, 2H), 2.17 (s, 1H), 1.96 (m, 1H), 1.71 (m, 1H), 1.24-1.30 (m, 3H). LC-MS m/z (M+H): 420.

### Step 9. (3S)-3-{[(tert-butyl(dimethyl)silyl]oxy}butyl-4-methylbenzenesulfonate

To a solution of methyl-(S)-3-hydroxybutyrate (15 g, 127 mmol) in anhydrous DMF (100 mL) under N₂ is added tert-butyldimethylsilyl chloride (21.1 g, 140 mmol), followed by imidazole (9.52 g, 140 mmol). The reaction mixture is stirred at room temperature overnight. The reaction is quenched with water (100 mL), and extracted with hexane. The organic phase is washed with water and brine, dried over Na₂SO₄. Removal of the solvent under reduced pressure affords a colorless oil which is dissolved in anhydrous THF (100 mL) and cooled to −78° C. DIBAL (381 mL, 1M in THF) is added slowly, and the reaction mixture is allowed to warm to room temperature overnight. The reaction mixture is cooled to 0° C., quenched with saturated sodium tartrate carefully, and then extracted with EtOAc (3×). The organic layers are combined, washed with brine, and dried over Na₂SO₄. Removal of the solvent under reduced pressure affords a colorless oil, which is dissolved in anhydrous CH₂Cl₂ (80 mL). TsCl (18.2, 95.6 mmol) is added in one portion, the mixture is cooled to 0° C., and pyridine (15.5 mL, 191.2 mmol) is added dropwise. The reaction mixture is stirred at room temperature overnight. The reaction is quenched with aqueous HCl (1N, 150 mL), and extracted with CH₂Cl₂. The organic phase is washed with saturated NaHCO₃ and brine, dried over Na₂SO₄, and concentrated under reduced pressure. The residue is treated with 30 g of ammonium carbonate resin in CH₂Cl₂ (200 mL) and MeOH (70 mL) at room temperature. After stirring for 3 h, the resin is removed via filtration through celite, the filtrate is concentrated under reduced pressure, and the residue is purified by flash chromatography on silica gel (hexane/EtOAc: 8/1) to afford the title compound as a colorless oil. ¹H-NMR (400 MHz, CDCl₃) δ: 7.78 (d, 2H), 7.33 (d, 2H), 4.10 (m, 2H), 3.89 (m, 1H), 2.44 (s, 3H), 1.68-1.77 (m, 2H), 1.09 (d, 3H), 0.80 (s, 9H), 0.01 (s, 3H), −0.03 (s, 3H). LC-MS m/z (M+Na): 381.

### Step 10. (2S)-4-{2,3-dimethyl-4-[(1S)-1-((1S,4S)-5-{[5-(trifluoromethyl)-2-pyridinyl]carbonyl}-2,5-diazabicyclo[2.2.1]hept-2-yl)ethyl]phenoxy}-2-butanol

To a suspension of 2,3-dimethylphenyl-4-[(1S)-[((1S,4S)-5-{[5-(trifluromethyl)pyridin-2-yl]carbonyl}-2,5-diazabicyclo[2.2.1]hept-2-yl)ethyl]phenol (3.0 g, 7.16 mmol) and Cs₂CO₃ (7.0 g, 21.4 mmol) in anhydrous DMF (30 mL) under N₂ is added (3S)-3-{[(tert-butyl(dimethyl)silyl]oxy}butyl-4-methylbenzenesulfonate compound (5.13 g, 14.32 mmol). The reaction mixture is stirred at 60° C. under N₂ overnight. The reaction is cooled to room temperature, diluted with 100 mL of water, and extracted with EtOAc (3×). The organic layer is washed with water (3×), brine, dried over Na₂SO₄, and concentrated under reduced pressure. The residue is purified by silica gel chromatography (hexane/EtOAc: 1/1) to afford ((1S,4S)-5-((S)-1-(4-((S)-3-(tert-butyldimethylsilyloxy)butoxy)-2,3-dimethylphenyl)ethyl)-2,5-diaza-bicyclo[2.2.1]-heptan-2-yl)(5-(trifluoromethyl)pyridin-2-yl)methanone as a yellow oil. The oil is dissolved in THF (30 mL), tetra n-butylammonium fluoride (9.7 mL, 1M in THF) is added at 0° C., and the mixture is stirred at room temperature overnight. The reaction mixture is diluted with 1:1 water-saturated brine and extracted with EtOAc. The organic layer is washed with water and brine, dried over Na₂SO₄, and concentrated under reduced pressure. The residue is purified by flash chromatography on silica gel (CH₂Cl₂/MeOH: 90/10) to afford the title compound as a yellow oil. ¹HNMR (400 MHz, CDCl₃) δ: 8.86 (s, 0.67H), 8.83 (s, 0.33H), 8.04-8.13 (m, 2H), 7.31 (m, 1H), 6.74 (m, 1H), 4.96 (s, 1H), 4.07-4.18 (m, 3H), 3.72-3.99 (m, 2H), 3.51 (m 1H), 3.35 (m, 1H), 3.20 (m, 1H), 2.82 (d, 0.67H), 2.77 (d, 0.33H), 2.33 (s, 2H), 2.25 (s, 1H), 2.22 (m, 1H), 2.18 (s, 2H), 2.16 (s, 1H), 1.92-1.98 (m, 3H), 1.71 (m, 1H), 1.25-1.30 (m, 6H). LC-MS m/z (M+H): 492.

### Example 18

### SYNTHESIS OF (2R)-1-{2,3-DIMETHYL-4-[(1S)-1-((1S,4S)-5-{[5-(TRIFLUOROMETHYL)-2-PYRIDINYL]CARBONYL}-2,5-DIAZABICYCLO[2.2.1]HEPT-2-YL)ETHYL]PHENOXY}-2-PROPANOL

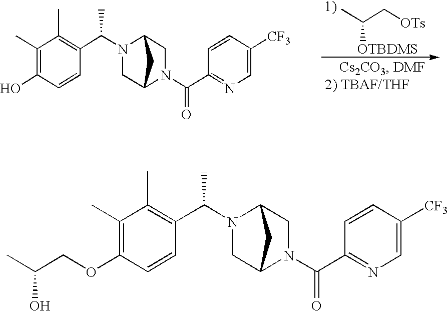

In an analogous manner to Example 17, the title compound is made from (2R)-2-{[(tert-butyl(dimethyl)silyl]oxy}-propyl-4-methylbenzenesulfonate (obtained from (R)-methyl 2-hydroxypropanoate via protection as the TBDMS ether and reduction with BH₃.THF) and 2,3-dimethylphenyl-4-[(1S)-1 ((1S,4S)-5-{[5-(trifluromethyl)pyridin-2-yl]carbonyl}-2,5-diazabicyclo-[2.2.1]hept-2-yl)ethyl]phenol, and is obtained as an off-white solid. ¹HNMR (400 MHz, CDCl₃) δ: 8.86 (s, 0.67H), 8.83 (s, 0.33H), 8.04-8.13 (m, 2H), 7.31 (m, 1H), 6.72 (m, 1H), 4.97 (s, 1H), 4.22 (m, 1H), 3.71-4.00 (m, 4H), 3.51 (m 1H), 3.35 (m, 1H), 3.23 (bs, 1H), 2.84 (d, 0.67H), 2.78 (d, 0.33H), 2.35 (m, 1H), 2.32 (s, 2H), 2.25 (s, 1H), 2.20 (s, 2H), 2.18 (s, 1H), 1.98 (m, 1H), 1.71 (m, 1H), 1.25-1.30 (m, 6H). LC-MS m/z (M+H): 478.

### Example 19

### SYNTHESIS OF (2R)-1-{2,3-DIMETHYL-4-[(1S)-1-((1S,4S)-5-{[6-(TRIFLUOROMETHYL)-3-PYRIDINYL]CARBONYL}-2,5-DIAZABICYCLO[2.2.1]HEPT-2-YL)ETHYL]PHENOXY}-2-PROPANOL

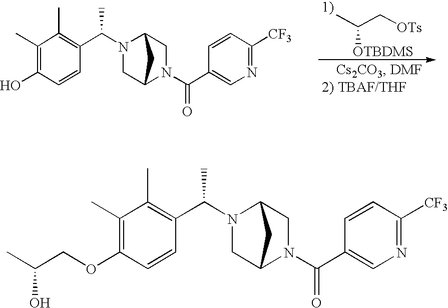

In an analogous manner to Example 17, the title compound is made from ((1S,4S)-5-((S)-1-(4-hydroxy-2,3-dimethylphenyl)ethyl)-2,5-diaza-bicyclo[2.2.1]heptan-2-yl)(6-(trifluoromethyl)pyridin-3-yl)methanone and (2R)-2-{[(tert-butyl(dimethyl)silyl]oxy}-propyl-4-methylbenzenesulfonate. The title compound is obtained as a yellow oil. ¹HNMR (400 MHz, CDCl₃) δ: 8.85 (m, 1H), 8.04 (m, 1H), 7.75 (m, 1H), 7.27 (m, 1H), 6.70 (m, 1H), 4.88 (s, 0.33H), 4.22 (m, 1H), 4.16 (s, 0.67H), 3.88-3.95 (m, 2H), 3.67-3.82 (m, 2H), 3.54 (m, 1H), 3.39 (m, 1H), 3.17 (m, 1H), 2.75 (m, 1H), 2.15-2.33 (m, 7H), 1.98 (m, 1H), 1.64-1.77 (m, 2H), 1.25-1.30 (m, 6H). LC-MS m/z (M+H): 478.

### Example 20

### SYNTHESIS OF (2S)-4-{2,3-DIMETHYL-4-[(1S)-1-((1S,4S)-5-{[6-(TRIFLUOROMETHYL)-3-PYRIDINYL]CARBONYL}-2,5-DIAZABICYCLO[2.2.1]HEPT-2-YL)ETHYL]PHENOXY}-2-BUTANOL

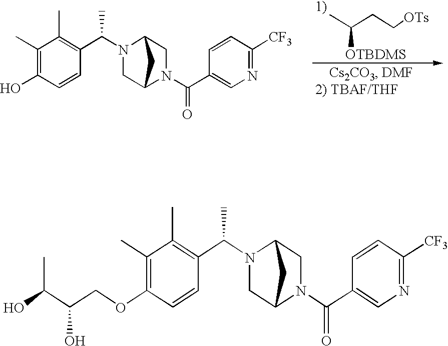

The title compound is made from 6-(trifluoromethyl)pyridine-3-carboxylic acid via a synthetic procedure similar to that described in Example 17. The title compound is obtained as a yellow oil. ¹HNMR (400 MHz, CDCl₃) δ: 8.84(m, 1H), 8.04 (m, 1H), 7.75 (m, 1H), 7.28 (m, 1H), 6.72 (m, 1H), 4.88 (s, 0.33H), 4.05-4.17 (m, 3.67H), 3.91 (m, 1H), 3.68 (m, 1H), 3.54 (m, 1H), 3.38 (m, 1H), 3.16 (m, 1H), 2.74 (m, 1H), 2.12-2.33 (m, 7H), 1.91-1.90 (m, 2H), 1.68-1.80 (m, 2H), 1.25-1.30 (m, 6H). LC-MS m/z (M+H): 492.

### Example 21

### SYNTHESIS OF (2R)-1-[4-((1S)-1-{(1S,4S)-5-[(6-ETHYL-3-PYRIDINYL)CARBONYL]-2,5-DIAZABICYCLO[2.2.1]HEPT-2-YL)ETHYL)-2,3-DIMETHYLPHENOXY]-2-PROPANOL

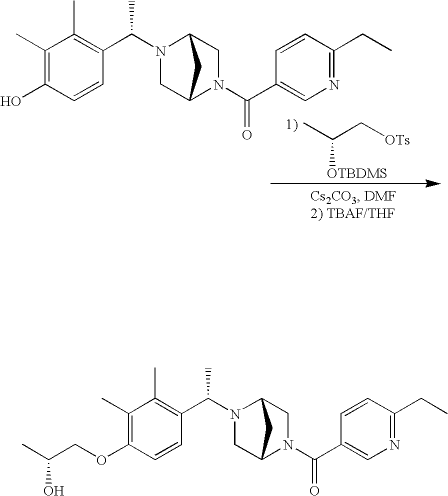

### Step 1. 6-Ethylpyridine-3-carboxylic acid

A flame-dried flask is charged under N₂ with methyl-6-chloronicotinate (11 g, 64.1 mmol), Fe(acac)₃ (1.13 g, 3.19 mmol), anhydrous THF (200 mL) and N-methylpyrrolidinone (20 mL). A solution of ethylmagnesium bromide (1M in THF, 76.9 mL, 76.9 mmol) is added. The resulting mixture is stirred for 10 min. The reaction is quenched with 1:1 water-saturated brine and extracted with EtOAc. The organic layer is washed with brine, dried over Na₂SO₄, and concentrated under reduced pressure. The residue is treated with aqueous NaOH (5N, 64 mL) and EtOH (64 mL) at room temperature overnight. EtOH is then removed under reduced pressure. The pH of the aqueous solution is adjusted to 4-5 with 6N HCl, and extracted with EtOAc. The organic layer is dried over Na₂SO₄ and concentrated under reduced pressure. The residue is purified by silica gel chromatography with EtOAc as the eluent to afford the title compound as a white solid. ¹HNMR (400 MHz, CDCl₃) δ: 9.29 (d, 1H), 8.37 (dd, 1H), 8.10 (bs, 1H), 7.34 (d, 1H), 2.99 (q, 2H), 1.37 (t, 3H). LC-MS m/z (M+H): 152.

### Step 2. (2R)-]-[4-((1S)-1-((1S,4S)-5-[(6-ethyl-3-pyridinyl)carbonyl]-2,5-diazabicyclo[2.2.1]hept-2-yl)ethyl)-2,3-dimethylphenoxy]-2-propanol

The title compound is made from (6-ethylpyridin-3-yl)((1S,4S)-5-((S)-1-(4-hydroxy-2,3-dimethylphenyl)ethyl)-2,5-diaza-bicyclo[2.2.1]heptan-2-yl)methanone and (2R)-2-{[(tert-butyl(dimethyl)silyl]oxy}-propyl-4-methylbenzenesulfonate via a synthetic procedure similar to that described in Example 17. The title compound is obtained as a yellow oil. ¹HNMR (400 MHz, CDCl₃) δ: 8.70 (s, 0.33H), 8.66 (s, 0.67H), 7.78 (m, 1H), 7.20-7.29 (m, 2H), 6.69 (m, 1H), 4.84 (s, 0.33H), 4.25(s, 0.67H), 4.22 (m, 1H), 3.87-3.91 (m, 2H), 3.34-3.82 (m, 4H), 3.18 (m, 1H), 2.86 (q, 2H), 2.79(d, 0.33H), 2.70(d, 0.67H), 2.42(d, 0.67H), 2.39(d, 0.33H), 2.33 (s, 2H), 2.19 (s, 2H), 2.15(s, 1H), 2.13(s, 1H), 1.95(m, 1H), 1.70(m, 1H), 1.23-1.34(m, 9H). LC-MS m/z (M+H): 438.

### Example 22

### SYNTHESIS OF 3-{2,3-DIMETHYL-4-[(1S)-1-((1S,4S)-5-{[5-(TRIFLUOROMETHYL)-2-PYRIDINYL]CARBONYL}-2,5-DIAZABICYCLO[2.2.1]HEPT-2-YL)ETHYL]PHENOXY}-N,N-DIMETHYL-1-PROPANAMINE

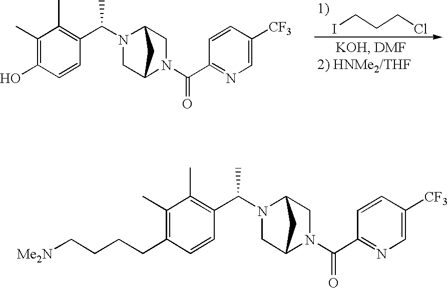

A mixture of 2,3-dimethylphenyl-4-[(1S)-1((1S,4S)-5-{[5-(trifluromethyl)pyridin-2-yl]carbonyl}-2,5-diazabicyclo-[2.2.1]hept-2-yl)ethyl]phenol (from Example 17, 350 mg, 0.83 mmol), Cs₂CO₃ (680 mg, 2.1 mmol) and 1-chloro-3-iodo-propane (135 μL, 1.25 mmol) in anhydrous DMF (5 mL) is stirred at room temperature under N₂ overnight. The reaction mixture is then diluted with EtOAc, washed with water (3×) and brine, dried over Na₂SO₄, and concentrated under reduced pressure. The residue is purified by passing through a silica gel plug (EtOAc/hexane: 1/1) to afford a yellow oil. To a solution of the oil (183 mg, 0.37 mmol) in DMA (3.7 mL) in a sealed tube is added DMA (3.7 mL, 2M in THF), Cs₂CO₃ (181 mg, 0.55 mmol) and catalytic amounts of NaI. The mixture is heated at 80° C. overnight. The reaction is cooled to room temperature, diluted with EtOAc, washed with water and brine, dried over Na₂SO₄, and concentrated under reduced pressure. The residue is purified by PTLC (CH₂Cl₂/MeOH/NH₄OH: 90/9/1) to yield the title compound as a yellow oil. ¹H NMR (CDCl₃, 300 MHz) δ: 8.86 (s, 0.67H), 8.82 (s, 0.33H), 8.0-8.13 (m, 2H), 7.30 (m, 1H), 6.71 (m, 1H), 4.96 (s, 1H), 3.71-4.02 (m, 4H), 3.52 (m, 1H), 3.34 (m, 1H), 3.21 (m, 1H), 2.79 (m, 1H), 2.54 (m, 2H), 2.33 (s), 2.32 (s), 2.24 (s), 2.17 (s), 2.15 (s) (total 12H), 1.94-2.08 (m, 3H), 1.69 (m, 1H), 1.28 (m, 3H). LC-MS (M+H): 505.

### Example 23

### SYNTHESIS OF 4-[2,3-DIMETHYL-4-((6R,9AS)-2-{[6-(TRIFLUOROMETHYL)-3-PYRIDINYL]CARBONYL}OCTAHYDRO-2H-PYRIDO[1,2-A]PYRAZIN-6-YL)PHENOXY]-N,N-DIMETHYL-1-BUTANAMINE

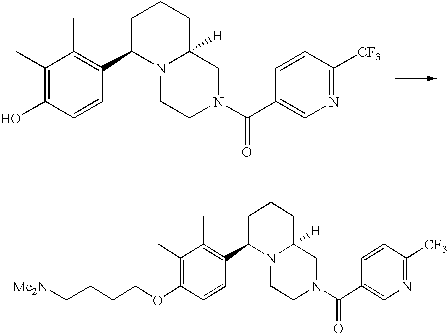

### Step 1. (6R,10S)-{6-[4-(4-bromobutoxy)-2,3-dimethyl-phenyl]octahydropyrido[1,2-a]pyrazine-2-yl}-(4-trifluoromethyl-3-pyridyl) methanone

A solution of (6R, 10S)-[6-(2,3-dimethyl-4-hydroxyphenyl)octahydropyrido[1,2-a]pyrazine-2-yl]-(4-trifluoromethyl-3-pyridyl) methanone (from Example 1, 455 mg, 1.05 mmoles) in DMF (4 mL) is treated with powdered Cs₂CO₃ (311 mg, 1.57 mmoles, 1.5 equiv.) and 1,4-dibromobutane (630 μL, 5.25 mmoles, 5.0 equiv.) and heated in a sealed tube reactor with stirring in a 70° C. oil bath for 20 h. The mixture is diluted with 25 mL water and extracted with CH₂Cl₂. Then combined extracts are dried over Na₂SO₄, filtered, and concentrated in vacuo. The residue is purified by silica gel column chromatography eluting with 60% hexanes/EtOAc to give the title compound as a brown oil. LC/MS: 568 (M+1)⁺.

### Step 2. 4-[2,3-Dimethyl-4-((6R,9aS)-2-([6-(trifluoromethyl)-3-pyridinyl]carbonyl}octahydro-2H-pyrido[1,2-a]pyrazin-6-yl)phenoxy]-N,N-dimethyl-1-butanamine

A solution of (6R, 10S)-{6-[4-(4-bromobutoxy)-2,3-dimethyl-phenyl]octahydro-pyrido[1,2-a]pyrazine-2-yl}-(4-trifluoromethyl-3-pyridyl) methanone (410 mg, 0.722 mmol) is dissolved in 3.0 mL of isopropyl alcohol, treated with 1.80 mL DMA (1 M in MeOH) and stirred in a 60° C. oil bath for 18 h. The reaction mixture is concentrated under reduced pressure and the residue purified by PTLC on a 2 mm silica plate eluting with 14% MeOH (2N NH₃)/CH₂Cl₂ to provide (6R, 10S)-{6-[4-(4-dimethylaminobutoxy)-2,3-dimethyl-phenyl]octahydropyrido[1,2-a]pyrazine-2-yl}-(4-trifluoromethyl-3-pyridyl) methanone as a brown foam. This is converted to the dihydrochloride salt by treating a CH₂Cl₂ solution of the free base with 2 equivalents 1M HCl in ether and concentrating. The free base is characterized as follows. LC/MS: 533 (M+1)⁺; ¹H NMR (mixture of rotamers, 400 MHz, CDCl₃) δ: 8.74 (1H, d), 7.93 (1H, dd), 7.73 (1H, dd), 7.30 (1H, dd), 6.70 (1H, dd), 4.52 (1H, dd), 4.06-3.90 (2H, m), 3.45-2.72 (5H, bm), 2.55 (3H, bm), 2.35, (7H, bm), 2.10-2.02 (8H, bm), 1.80 (8H, bm), 1.26 (3H, bm).

### Example 24

### SYNTHESIS OF 3-[2,3-DIMETHYL-4-((6R,9AS)-2-{[6-(TRIFLUOROMETHYL)PYRIDIN-3-YL]CARBONYL}OCTAHYDRO-2H-PYRIDO[1,2-A]PYRAZIN-6-YL)PHENOXY]-N,N-DIMETHYLPROPAN-1-AMINE

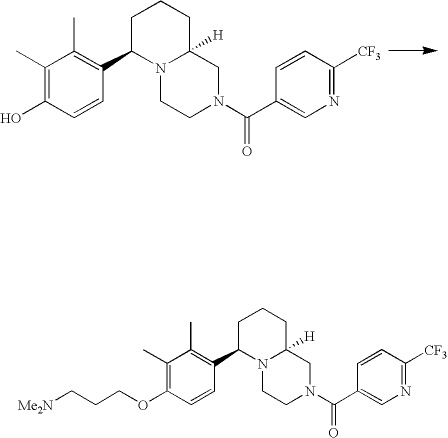

This compound is made by a procedure analogous to Example 23, replacing 1,4-dibromobutane with 1-chloro-3-iodo-propane in step 1 of the synthesis. LC/MS: 519 (M+1)⁺.

### Example 25

### SYNTHESIS OF 3-[2,3-DIMETHYL-4-((6R,9AS)-2-{[2-(TRIFLUOROMETHYL)-5-PYRIMIDINYL]CARBONYL}OCTAHYDRO-2H-PYRIDO[1,2-A]PYRAZIN-6-YL)PHENOXY]-N-(2-METHOXYETHYL)-1-PROPANAMINE

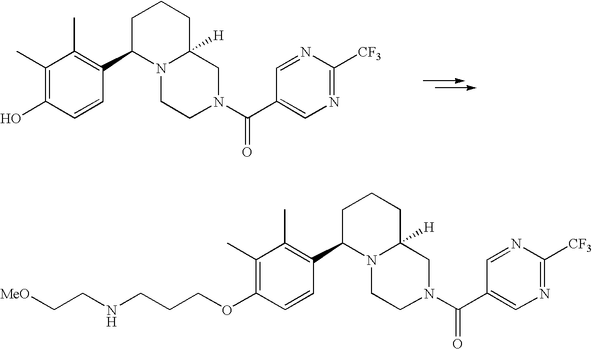

### Step 1. 2,3-dimethyl-4-((6R,9aS)-2-{[2-(trifluoromethyl)-5-pyrimidinyl]carbonyl}octahydro-2H-pyrido[1,2-a]pyrazin-6-yl)phenol

The title compound is made by a procedure analogous to that for 2,3-dimethyl-4-((6R,9aS)2-{[6-(trifluoromethyl)-3-pyridinyl]carbonyl}octahydro-2H-pyrido[1,2-a]pyrazin-6-yl)phenol, replacing 6-trifluoromethylnicotinic acid with 2-(trifluoromethyl)pyrimidine-5-carboxylic acid.

### Step 2. 3-[2,3-dimethyl-4-((6R,9aS)-2-{[2-(trifluoromethyl)-5-pyrimidinyl]carbonyl}octahydro-2H-pyrido[1,2-a]pyrazin-6-yl)phenoxy]-N-(2-methoxyethyl)-1-chloro-propane

To a solution of 2,3-dimethyl-4-((6R,9aS)-2-{[2-(trifluoromethyl)-5-pyrimidinyl]carbonyl}octahydro-2H-pyrido[1,2-a]pyrazin-6-yl)phenol (2.3 g, 5.29 mmol) in 2-butanone (50 mL) is added Cs₂CO₃ (1.89 g, 5.82 mmol) under N₂ atmosphere, and the resulting mixture is stirred for 25 min at room temperature. 1-Chloro-3-iodo-propane (3.2 g, 15.6 mmol) and catalytic amounts of KI are added, and the resulting mixture is stirred at 80° C. for 16 h. The reaction mixture is taken to room temperature, diluted with 100 mL of EtOAc and washed with water, brine, dried over Na₂SO₄, and concentrated under reduced pressure. The residue is purified by flash chromatography eluting with 30% EtOAc-hexanes to afford the title compound as a foamy solid. ¹H NMR (300 MHz, CDCl₃): δ 8.95 (s, 1H), 8.90 (s, 1H), 7.33-7.35 (m, 1H), 6.70-6.78 (m, 1H), 4.51 (q, J=13.8 Hz, 1H), 4.04-4.10 (m, 2H), 3.76 (q, J=6 Hz, 2H), 3.28-3.40 (m, 4H), 2.53-2.90 (m, 3H), 2.14-2.29 (m containing two s at 2.20, 2.17, 8H), 1.73-1.90 (m, 3H), 1.32-1.58 (m, 3H). LC-MS found 511 (M+1)⁺.

### Step 3. 3-[2,3-dimethyl-4-((6R,9aS)-2-{[2-(trifluoromethyl)-5-pyrimidinyl]carbonyl}octahydro-2H-pyrido[1,2-a]pyrazin-6-yl)phenoxy]-N-(2-methoxyethyl)-1-propanamine

To a solution of 3-[2,3-dimethyl-4-((6R,9aS)-2-{[2-(trifluoromethyl)-5-pyrimidinyl]-carbonyl}octahydro-2H-pyrido[1,2-a]pyrazin-6-yl)phenoxy]-N-(2-methoxyethyl)-1-chloro-propane (2 g, 3.92 mmol) in anhydrous acetonitrile (30 mL) are added Cs₂CO₃ (1.27 g, 3.92 mmol), 2-methoxyethylamine (3.37 mL, 39.12 mmol) and catalytic KI under N₂ atmosphere. The resulting mixture is stirred at 80° C. for 16 h. The reaction mixture is cooled to room temperature, filtered, washed with EtOAc and concentrated under reduced pressure. The residue is purified by flash chromatography eluting with 2-5% MeOH—CH₂Cl₂ containing 0.5% NH₄OH to afford the title compound as a foamy solid. ¹H NMR (300 MHz, CDCl₃): δ 8.96 (s, 1H), 8.90 (s, 1H), 7.30-7.36 (m, 1H), 6.69-6.77 (m, 1H), 4.51 (q, J=13.8 Hz, 1H), 4.03-4.11 (m, 2H), 3.44-3.68 (m, 3H), 3.31-3.37 (m containing s at 3.35, 5H), 2.95-3.16 (m, 9H), 2.14-2.24 (m containing two s at 2.21, 2.14, 8H), 1.66-1.92 (m, 3H), 1.42-1.54 (m, 3H); LC/MS found 550 (M+1)⁺.

### Example 26

### SYNTHESIS OF 3-[2,3-DIMETHYL-4-((6R,9AS)-2-{[2-(TRIFLUOROMETHYL)-5-PYRIMIDINYL]-CARBONYL}OCTAHYDRO-2H-PYRIDO[1,2-A]PYRAZIN-6-YL)PHENOXY]-N,N-DIMETHYL-1-PROPANAMINE

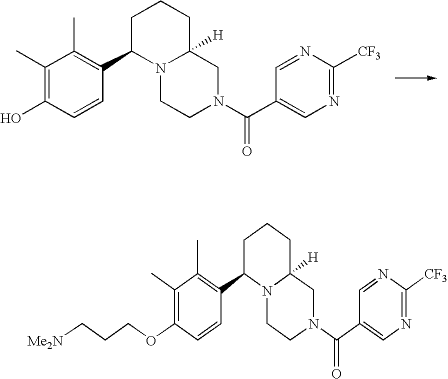

This compound is made by a procedure analogous to Example 25, replacing 2-methoxyethylamine with equivalent amounts of dimethylamine in step 2 of the synthesis. LC/MS: 520 (M+1)⁺.

### Example 27

### SYNTHESIS OF 3-[2,3-DIMETHYL-4-((6R,9AS)-2-{[2-(TRIFLUOROMETHYL)-5-PYRIMIDINYL]CARBONYL}OCTAHYDRO-2H-PYRIDO[1,2-A]PYRAZIN-6-YL)PHENOXY]-N-(2-METHOXYETHYL)-1-PROPANAMINE

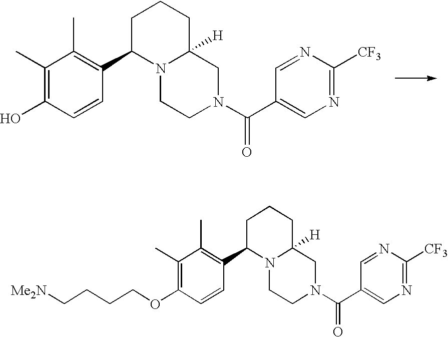

### Step 1. 4-[2,3-dimethyl-4-((6R,9aS)-2-{[2-(trifluoromethyl)-5-pyrimidinyl]carbonyl}octa-hydro-2H-pyrido[1,2-a]pyrazin-6-yl)phenoxy]-]-chloro-butane

The title compound is prepared following the same protocol used in Example 25, step 1, using 1-chloro-4-iodo-butane as the alkylating agent. ¹H NMR (300 MHz, CDCl₃): [symbol] 8.95 (s, 1H), 8.90 (s, 1H), 7.29-7.34 (m, 1H), 6.70-6.75 (m, 1H), 4.50 (q, J=14.7 Hz, 1H), LC-MS found 525 (M+1)⁺.

### Step 2. 3-[2,3-dimethyl-4-((6R,9aS)-2-{[2-(trifluoromethyl)-5-pyrimidinyl]carbonyl}octa-hydro-2H-pyrido[1,2-a]pyrazin-6-yl)phenoxy]-N-(2-methoxyethyl)-1-propanamine

The title compound is prepared by reacting 4-[2,3-dimethyl-4-((6R,9aS)-2-{[2-(trifluoromethyl)-5-pyrimidinyl]carbonyl}octa-hydro-2H-pyrido[1,2-a]pyrazin-6-yl)phenoxy]-1-chloro-butane with dimethylamine following the method described in Example 26. ¹H NMR (300 MHz, CDCl₃): δ 8.95 (s, 1H), 8.90 (s, 1H), 7.29-7.34 (m, 1H), 6.66-6.73 (m, 1H), 4.50 (q, J=13.5 Hz, 1H), 3.85-4.11 (m, 2H), 3.13-3.39 (m, 3H), 2.68-2.90 (m, 4H), 2.45-2.65 (m containing two s at 2.56, 7H), 2.13-2.20 (m, 7H), 1.70-1.97 (m, 7H), 1.35-1.54 (m, 3H); LC-MS found 534 (MH+).

### Example 28

### SYNTHESIS OF 4-[2,3-DIMETHYL-4-((6R,9AS)-2-{[2-(TRIFLUOROMETHYL)-5-PYRIMIDINYL]CARBONYL}OCTAHYDRO-2H-PYRIDO[1,2-A]PYRAZIN-6-YL)PHENOXY]-N-(2-METHOXYETHYL)-N-METHYL-1-BUTANAMINE

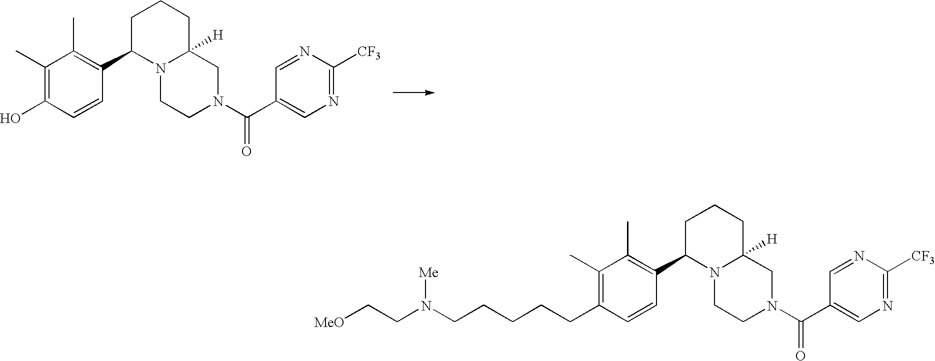

The title compound is prepared by reacting 4-[2,3-dimethyl-4-((6R,9aS)-2-{[2-(trifluoromethyl)-5-pyrimidinyl]carbonyl}octahydro-2H-pyrido[1,2-a]pyrazin-6-yl)phenoxy]-1-chloro-butane (obtained in Example 27) with equivalent amounts of (2-methoxy-ethyl)-methyl-amine following the method described in Example 26. ¹H NMR (300 MHz, CDCl₃): δ 8.95 (s, 1H), 8.90 (s, 1H), 7.27-7.33 (m, 1H), 6.66-6.74 (m, 1H), 4.50 (q, J=12.6 Hz, 1H), 3.92-4.05 (m, 2H), 3.46-3.48 (m, 2H), 3.24-3.42 (m containing s at 3.34, 5H), 3.08-3.18 (m, 1H), 2.66-2.96 (m, 2H), 2.45-2.58 (m, 4H), 2.14-2.28 (m, contains s at 2.28, 11H), 1.69-1.92 (m, 7H), 1.38-1.6 (m, 3H); LC-MS found 578 (M+1)⁺.

### Example 29

### SYNTHESIS OF 2-[2,3-DIMETHYL-4-((6R,9AS)-2-{[2-(TRIFLUOROMETHYL)-5-PYRIMIDINYL]CARBONYL}OCTAHYDRO-2H-PYRIDO[1,2-A]PYRAZIN-6-YL)PHENOXY]ETHANOL

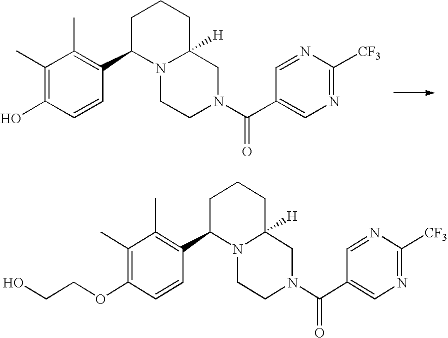

To a solution of 2,3-dimethyl-4-((6R,9aS)-2-{[2-(trifluoromethyl)-5-pyrimidinyl]-carbonyl}octahydro-2H-pyrido[1,2-a]pyrazin-6-yl)phenol (2 g, 4.6 mmol) in anhydrous DMF (20 mL) is added Cs₂CO₃ (3.7 g, 11.3 mmol) under N₂ atmosphere, and the resulting mixture is stirred for 25 min. (2-Bromo ethoxy)-tert-butyl dimethyl silane (2.19 g, 9.2 mmol) and catalytic amounts of KI are added and the resulting mixture is stirred at 60° C. for 16 h. The reaction mixture is cooled to room temperature and diluted with 100 mL of water, extracted with EtOAc, and the organic layers are washed with water, brine, dried over Na₂SO₄, and concentrated. The crude product is dissolved in 20 mL of anhydrous THF, 9 mL of 1.0 N TBAF (9.2 mmol) is added dropwise and the reaction mixture is stirred for 4 h at room temperature. The reaction mixture is concentrated under reduced pressure diluted with Et₂O (100 mL), washed with brine (2×), dried over Na₂SO₄, and concentrated under reduced pressure. The residue obtained is purified by flash chromatography on silicagel eluting with 60% EtOAc-hexane to afford the desired product as a foamy solid. ¹H NMR (300 MHz, CDCl₃): δ 8.94 (s, 1H), 8.89 (s, 1H), 7.25-7.34 (m, 1H), 6.70-6.78 (m, 1H), 4.49 (q, J=14.4 Hz, 1H), 4.04-4.13 (m, 2H), 3.78-3.92 (m, 2H), 3.27-3.38 (m, 2H), 3.13 (t, J=14.4 Hz, 1H), 2.67-2.93 (m, 2H), 2.51 (br t, 1H), 2.01-2.15 (m, 6H), 1.61-1.93 (m, 2H), 1.34-1.57 (m, 21); LC-MS found 479 (M+H)⁺.

### Example 30

### SYNTHESIS OF (2R)-1-[2,3-DIMETHYL-4-((6R,9AS)-2-{[2-(TRIFLUOROMETHYL)-5-PYRIMIDINYL]CARBONYL}OCTAHYDRO-2H-PYRIDO[1,2-A]PYRAZIN-6-YL)PHENOXY]-2-PROPANOL

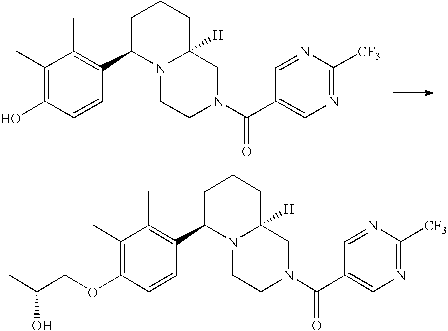

The title compound is prepared following the protocol used to prepare [2,3-dimethyl-4-((6R,9aS)-2-{[2-(trifluoromethyl)-5-pyrimidinyl]carbonyl}octahydro-2H-pyrido[1,2-a]pyrazin-6-yl)phenoxy]ethanol, using (2R)-2-{[(tert-butyl(dimethyl)silyl]oxy}-propyl-4-methylbenzene-sulfonate (as in Example 21). ¹H NMR (300 MHz, CDCl₃): δ 8.95 (s, 1H), 8.90 (s, 1H), 7.33-7.36 (m, 1H), 6.68-6.76 (m, 1H), 4.50 (q, J=15.6 Hz, 1H), 4.12-4.28 (m, 1H), 3.73-3.95 (m, 2H), 3.09-3.39 (m, 3H), 2.67-2.90 (m, 2H), 2.21-2.58 (m, 2H) 2.19 (s,3H), 2.16 (s, 3H), 1.65-1.92 (m, 2H), 1.38-1.54 (m, 2H), 1.25-1.30 (m, 2H); LC-MS found 493 (M+H)⁺.

### Example 31

### Additional Aryl-Substituted Piperazine Derivatives

Additional representative aryl-substituted piperazine derivatives are shown in the following table, and are prepared according to the methods presented in the foregoing Schemes and Examples. The compounds in Tables I-DX satisfy at least one of the following criteria:
(i) exhibit an EC₅₀ of less than 1 micromolar in the calcium mobilization assay of Example 37; and/or
(ii) exhibit an EC₅₀ of less than 1 micromolar in the GTP binding assay of Example 35.

Mass spectroscopy data in the “MS” column is obtained as described above and presented as (M+1).

TABLE I

| | COMPOUND | NAME | MS |
|---|---|---|---|
| 1 | 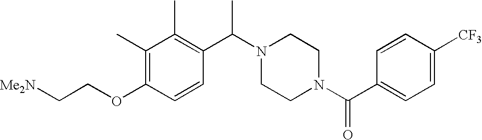 | 2-[2,3-dimethyl-4-(1-{4-[4-(tri- fluoromethyl)benzoyl]piperazin-1-yl}eth- yl)phenoxy]-N,N-di- methylethanamine | 478 |
| 2 | 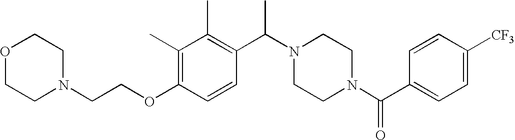 | 4-{2-[2,3-dimethyl-4-(1-{4-[4-(tri- fluoromethyl)benzoyl]piperazin-1-yl}eth- yl)phenoxy]ethyl}-morpholine | 520 |
| 3 | 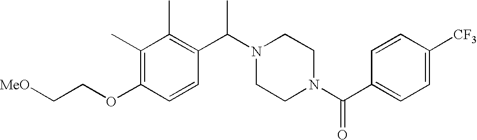 | 1-{1-[4-(2-methoxyethoxy)-2,3-di- methylphenyl]ethyl}-4-[4-(tri- fluoromethyl)benzoyl]piperazine | 465 |
| 4 | 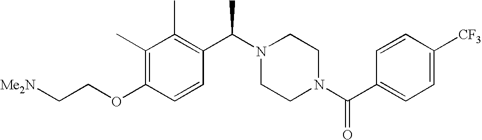 | 2-[2,3-dimethyl-4-((1R)-1-{4-[4-(tri- fluoromethyl)benzoyl]piperazin-1-yl}eth- yl)phenoxy]-N,N-di- methylethanamine | 478 |
| 5 | 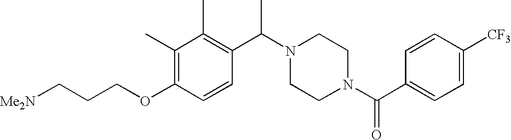 | 3-[2,3-dimethyl-4-(1-{4-[4-(tri- fluoromethyl)benzoyl]piperazin-1-yl}eth- yl)phenoxy]-N,N-di- methylpropan-1-amine | 492 |
| 6 | 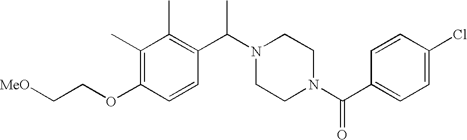 | 1-(4-chlorobenzoyl)-4-{1-[4-(2-meth- oxyethoxy)-2,3-di- methylphenyl]ethyl}piperazine | 431 |
| 7 | 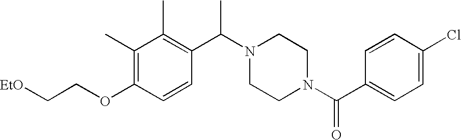 | 1-(4-chlorobenzoyl)-4-{1-[4-(2-eth- oxyethoxy)-2,3-di- methylphenyl]ethyl}piperazine | 445 |
| 8 | 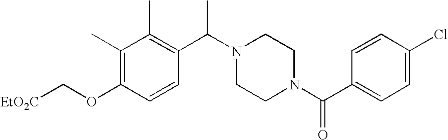 | ethyl(4-{1-[4-(4-chloro- benzoyl)piperazin-1-yl]ethyl}-2,3-di- methylphenoxy)acetate | 459 |
| 9 | 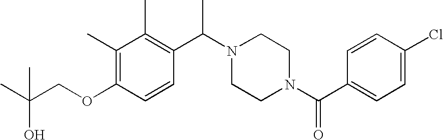 | 1-(4-{1-[4-(4-chloro- benzoyl)piperazin-1-yl]ethyl}-2,3-di- methylphenoxy)-2-meth- ylpropan-2-ol | 445 |
| 10 | 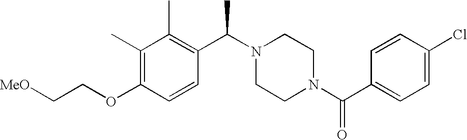 | 1-(4-chlorobenzoyl)-4-{(1R)-1-[4-(2-meth- oxyethoxy)-2,3-di- methylphenyl]ethyl}piperazine | 431 |
| 11 | 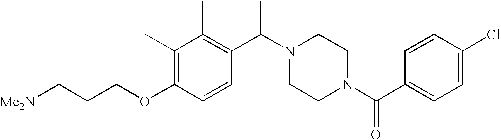 | 3-(4-{1-[4-(4-chloro- benzoyl)piperazin-1-yl]ethyl}-2,3-di- methylphenoxy)-N,N-di- methylpropan-1-amine | 458 |
| 12 | 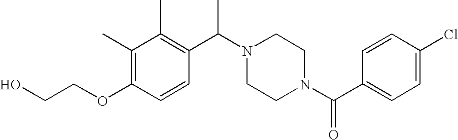 | 2-(4-{1-[4-(4-chloro- benzoyl)piperazin-1-yl]ethyl}-2,3-di- methylphenoxy)ethanol | 417 |
| 13 | 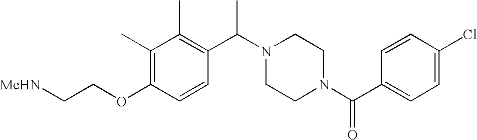 | 2-(4-{1-[4-(4-chloro- benzoyl)piperazin-1-yl]ethyl}-2,3-di- methylphenoxy)-N-meth- ylethanamine | 430 |
| 14 | 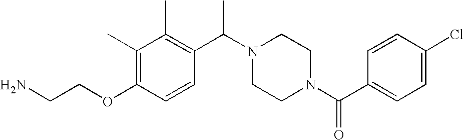 | 2-(4-{1-[4-(4-chloro- benzoyl)piperazin-1-yl]ethyl}-2,3-di- methylphenoxy)ethanamine | 416 |
| 15 | 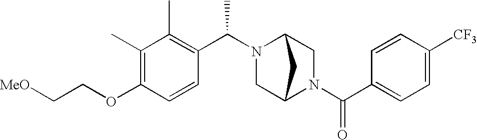 | (1S,4S)-2-{(1R)-1-[4-(2-meth- oxyethoxy)-2,3-di- methylphenyl]ethyl}-5-[4-(tri- fluoromethyl)benzoyl]-2,5-diaza- bicyclo[2.2.1]heptane | 477 |
| 16 | 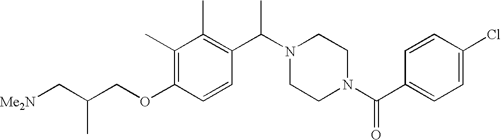 | 1-(4-chlorobenzoyl)-4-((1R)-1-{2,3-di- methyl-4-[2-(1-methylpyrrolidin-2-yl)eth- oxy]phenyl}ethyl)piperazine | 472 |
| 17 | 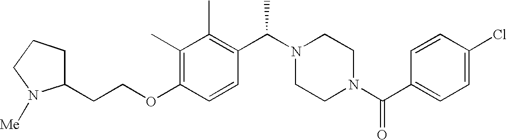 | 1-(4-chlorobenzoyl)-4-((1S)-1-{2,3-di- methyl-4-[2-(1-meth- ylpyrrolidin-2-yl)eth- oxy]phenyl}ethyl)piperazine | 484 |
| 18 | 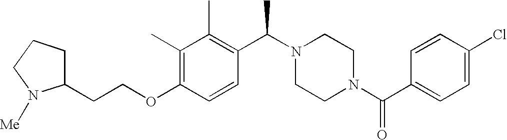 | 3-[2,3-dimethyl-4-(1-{(1S,4S)-5-[4-(tri- fluoromethyl)benzoyl]-2,5-di- azabicyclo[2.2.1]hept-2-yl}eth- yl)phenoxy]-N,N-di- methylpropan-1-amine | 484 |
| 19 | 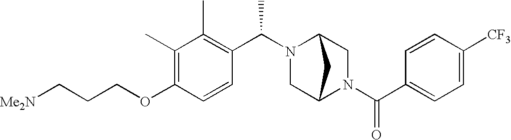 | 2-[2,3-dimethyl-4-(1-{4-[4-(tri- fluoromethyl)benzoyl]piperazin-1-yl}eth- yl)phenoxy]-N,N-di- methylethanamine | 504 |
| 20 | 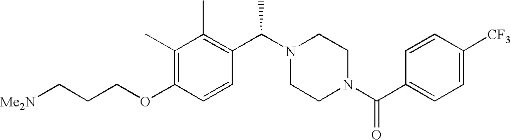 | 3-[2,3-dimethyl-4-((1S)-1-{4-[4-(tri- fluoromethyl)benzoyl]-piperazin-1-yl}eth- yl)phenoxy]-N,N-di- methylpropan-1-amine | 492 |
| 21 | 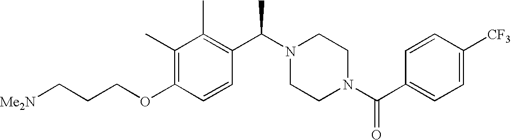 | 3-[2,3-dimethyl-4-((1R)-1-{4-[4-(tri- fluoromethyl)benzoyl]-piperazin-1-yl}eth- yl)phenoxy]-N,N-di- methylpropan-1-amine | 492 |
| 22 | 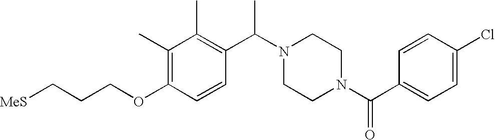 | 1-(4-chlorobenzoyl)-4-(1-{2,3-di- methyl-4-[3-(methylthio)-pro- poxy]phenyl}ethyl)piperazine | 461 |
| 23 | 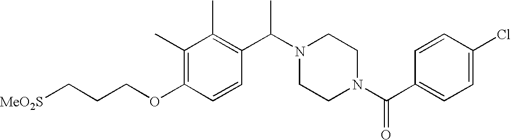 | 1-(4-chlorobenzoyl)-4-(1-{2,3-di- methyl-4-[3-(methylsulfonyl)-pro- poxy]phenyl}ethyl)piperazine | 493 |
| 24 | 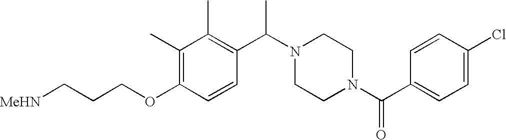 | 3-(4-{1-[4-(4-chloro- benzoyl)piperazin-1-yl]ethyl}-2,3-di- methylphenoxy)-N-meth- ylpropan-1-amine | 444 |
| 25 | 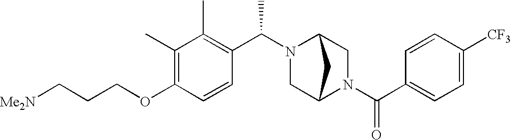 | 3-[2,3-dimethyl-4-((1S)-1-{(1S,4S)-5-[4-(tri- fluoromethyl)benzoyl]-2,5-diaza- bicyclo[2.2.1]hept-2-yl}eth- yl)phenoxy]-N,N-di- methylpropan-1-amine | 504 |
| 26 | 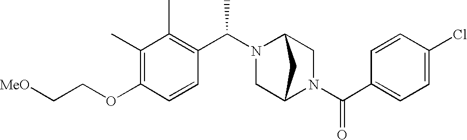 | (1R,4R)-2-(4-chlorobenzoyl)-5-{(1R)-1-[4-(2-meth- oxyethoxy)-2,3-di- methylphenyl]ethyl}-2,5-diaza- bicyclo[2.2.1]heptane | 443 |
| 27 | 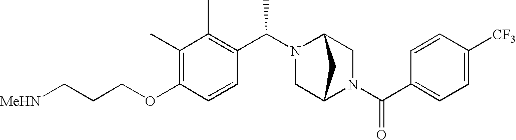 | 3-[2,3-dimethyl-4-((1S)-1-{(1S,4S)-5-[4-(tri- fluoromethyl)benzoyl]-2,5-diaza- bicyclo[2.2.1]hept-2-yl}eth- yl)phenoxy]-N-methylpropan-1-amine | 490 |
| 28 | 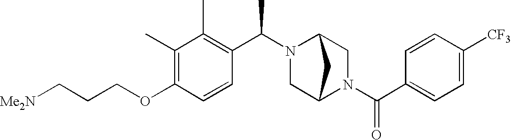 | 3-[2,3-dimethyl-4-((1R)-1-{(1S,4S)-5-[4-(tri- fluoromethyl)benzoyl]-2,5-diaza- bicyclo[2.2.1]hept-2-yl}eth- yl)phenoxy]-N,N-di- methylpropan-1-amine | 504 |
| 29 | 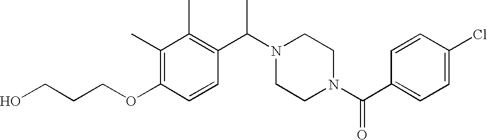 | 3-(4-{1-[4-(4-chloro-ben- zoyl)piperazin-1-yl]ethyl}-2,3-di- methylphenoxy)propan-1-ol | 431 |
| 30 | 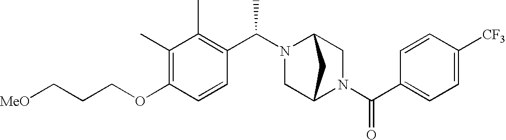 | (1R,4R)-2-{(1R)-1-[4-(3-meth- oxypropoxy)-2,3-di- methylphenyl]ethyl}-5-[4-(tri- fluoromethyl)benzoyl]-2,5-diaza- bicyclo[2.2.1]heptane | 491 |
| 31 | 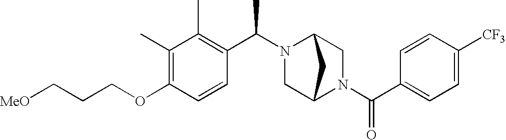 | (1R,4R)-2-{(1S)-1-[4-(3-meth- oxypropoxy)-2,3-di- methylphenyl]ethyl}-5-[4-(tri- fluoromethyl)benzoyl]-2,5-diaza- bicyclo[2.2.1]heptane | 491 |
| 32 | 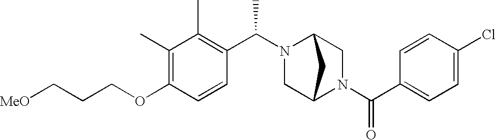 | (1R,4R)-2-(4-chlorobenzoyl)-5-{(1R)-1-[4-(3-meth- oxypropoxy)-2,3-di- methylphenyl]ethyl}-2,5-diaza- bicyclo[2.2.1]heptane | 457 |
| 33 | 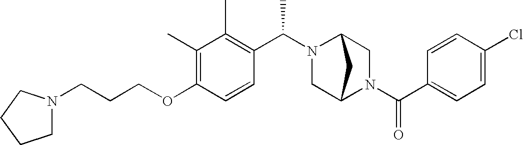 | (1R,4R)-2-(4-chlorobenzoyl)-5-{(1R)-[2,3-di- methyl-4-(3-pyrrolidin-1-yl- propoxy)-phenyl]ethyl}-2,5-diaza- bicyclo-[2.2.1]heptane | 496 |
| 34 | 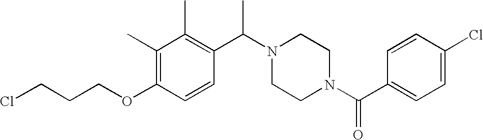 | 1-(4-chlorobenzoyl)-4-{1-[4-(3-chloro- propoxy)-2,3-di- methylphenyl]ethyl}piperazine | 449 |
| 35 | 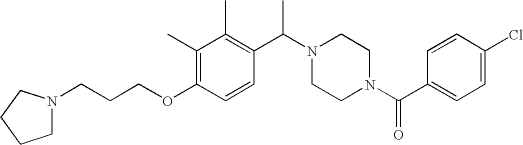 | 1-(4-chlorobenzoyl)-4-{1-[2,3-di- methyl-4-(3-pyrrolidin-1-yl- propoxy)phenyl]ethyl}piperazine | 484 |
| 36 | 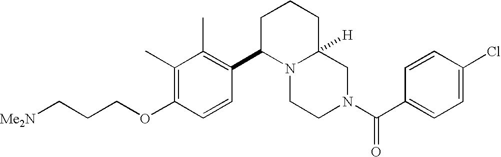 | 3-{4-[(6R,9aS)-2-(4-chloro- benzoyl)octahydro-2H-py- rido[1,2-α]pyrazin-6-yl]-2,3-di- methylphenoxy}-N,N-di- methylpropan-1-amine | 518 |
| 37 | 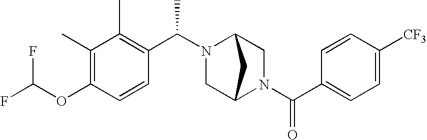 | (1S,4S)-2-{(1S)-1-[4-(di- fluoromethoxy)-2,3-di- methylphenyl]ethyl}-5-[4-(tri- fluoromethyl)benzoyl]-2,5-diaza- bicyclo-[2.2.1]heptane | 469 |
| 38 | 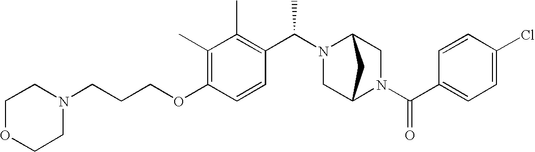 | (1R,4R)-2-(4-chlorobenzoyl)-5-{(1S)-[2,3-di- methyl-4-(3-morpholin-4-yl- propoxy)-phenyl]ethyl}-2,5-diaza- bicyclo-[2.2.1]heptane | 512 |
| 39 | 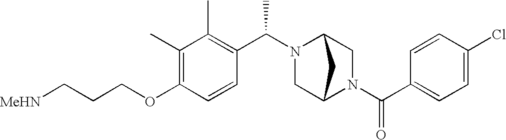 | 3-(4-{(1S)-1-[(1S,4S)-5-(4-chloro- benzoyl)-2,5-diaza- bicyclo[2.2.1]hept-2-yl]ethyl}-2,3-di- methylphenoxy)-N-methyl- propan-1-amine | 456 |
| 40 | 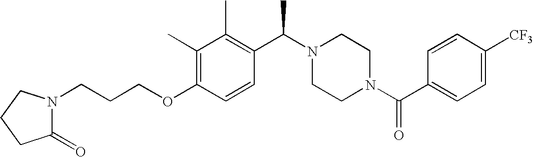 | 1-{3-[2,3-dimethyl-4-((1R)-1-{4-[4-(tri- fluoromethyl)benzoyl]-piperazin-1-yl}eth- yl)-phenoxy]propyl}pyrrolidin-2-one | 532 |
| 41 | 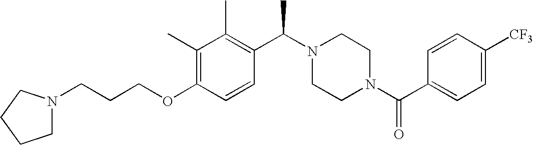 | 1-{(1R)-1-[2,3-dimethyl-4-(3-pyr- rolidin-1-ylpropoxy)phenyl]ethyl}-4-[4-(tri- fluoromethyl)benzoyl]piperazine | 518 |
| 42 | 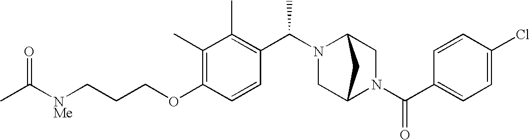 | N-[3-(4-{(1S)-1-[(1S,4S)-5-(4-chloro- benzoyl)-2,5-diaza- bicyclo[2.2.1]hept-2-yl]ethyl}-2,3-di- methylphenoxy)propyl]-N-methyl- acetamide | 498 |
| 43 | 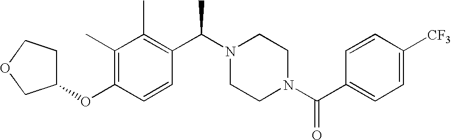 | 1-((1R)-1-{2,3-dimethyl-4-[(3S)-tetra- hydrofuran-3-yloxy]-phe- nyl}ethyl)-4-[4-(tri- fluoromethyl)benzoyl]piperazine | 477 |
| 44 | 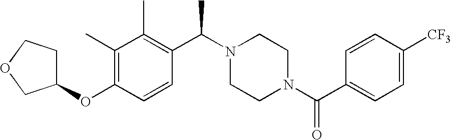 | 1-((1R)-1-{2,3-dimethyl-4-[(3R)-tetra- hydrofuran-3-yl- oxy]phenyl}ethyl)-4-[4-(tri- fluoromethyl)benzoyl]piperazine | 477 |
| 45 | 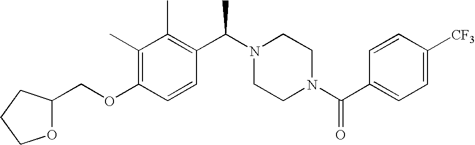 | 1-{(1R)-1-[2,3-dimethyl-4-(tetra- hydrofuran-2-yl- methoxy)phenyl]ethyl}-4-[4-(tri- fluoromethyl)benzoyl]piperazine | 477 |
| 46 | 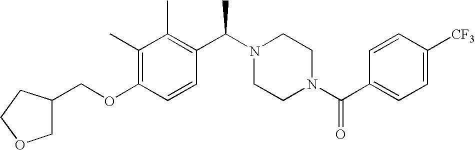 | 1-{(1R)-1-[2,3-dimethyl-4-(tetra- hydrofuran-3-ylmethoxy)-phe- nyl]ethyl}-4-[4-(trifluoro-meth- yl)benzoyl]piperazine | 491 |
| 47 | 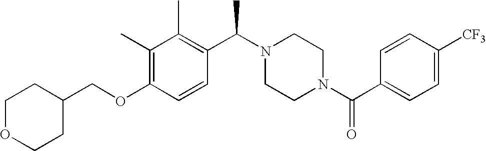 | 1-{(1R)-1-[2,3-dimethyl-4-(tetrahydro-2H-py- ran-4-yloxy)-phenyl]ethyl}-4-[4-(tri- fluoromethyl)benzoyl]piperazine | 491 |
| 48 | 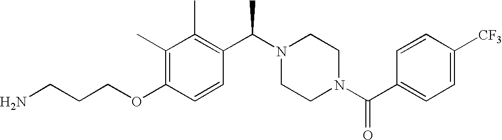 | 3-[2,3-dimethyl-4-((1R)-1-{4-[4-(tri- fluoromethyl)benzoyl]piperazin-1-yl}eth- yl)phenoxy]propan-1-amine | 464 |
| 49 | 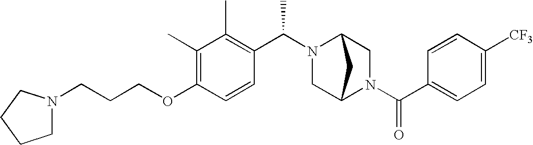 | (1S,4S)-2-{(1S)-1-[2,3-dimethyl-4-(3-pyr- rolidin-1-ylpropoxy)-phe- nyl]ethyl}-5-[4-(trifluoro- methyl)benzoyl]-2,5-diaza- bicyclo[2.2.1]heptane | 530 |
| 50 | 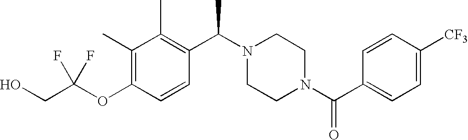 | 2-[2,3-dimethyl-4-((1R)-1-{4-[4-(tri- fluoromethyl)benzoyl]piperazin-1-yl}eth- yl)phenoxy]-2,2-difluoroethanol | 487 |
| 51 | 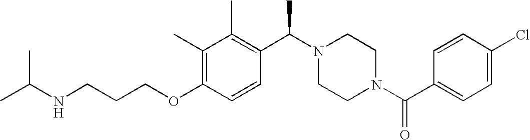 | 3-(4-{(1R)-1-[4-(4-chloro- benzoyl)piperazin-1-yl]ethyl}-2,3-di- methylphenoxy)-N-iso- propylpropan-1-amine | 472 |
| 52 | 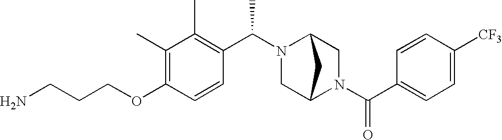 | 3-[2,3-dimethyl-4-((1S)-1-{(1S,4S)-5-[4-(tri- fluoromethyl)benzoyl]-2,5-diaza- bicyclo[2.2.1]hept-2-yl}eth- yl)phenoxy]propan-1-amine | 476 |
| 53 | 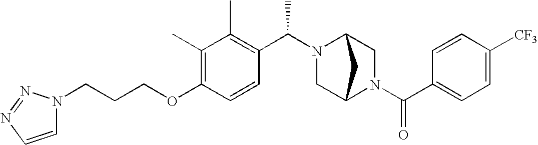 | (1S,4S)-2-((1S)-1-{2,3-dimethyl-4-[3-(1H-1,2,3-tri- azol-1-yl)pro- poxy]phenyl}ethyl)-5-[4-(tri- fluoromethyl)benzoyl]-2,5-diaza- bicyclo[2.2.1]heptane | 528 |
| 54 | 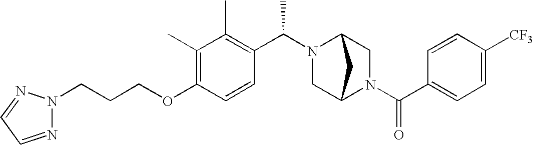 | (1S,4S)-2-((1R)-1-{2,3-dimethyl-4-[3-(2H-1,2,3-tri- azol-2-yl)pro- poxy]phenyl}ethyl)-5-[4-(tri- fluoromethyl)benzoyl]-2,5-diaza- bicyclo[2.2.1]heptane | 520 |
| 55 | 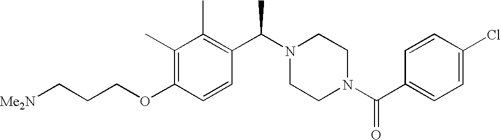 | 3-(4-{(1R)-1-[4-(4-chloro- benzoyl)piperazin-1-yl]ethyl}-2,3-di- methylphenoxy)-N,N-di- methylpropan-1-amine | 458 |
| 56 | 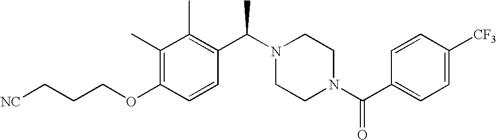 | 4-[2,3-dimethyl-4-((1R)-1-{4-[4-(tri- fluoromethyl)benzoyl]piperazin-1-yl}eth- yl)phenoxy]butanenitrile | 474 |
| 57 | 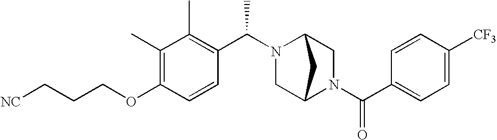 | 4-[2,3-dimethyl-4-((1S)-1-{(1S,4S)-5-[4-(tri- fluoromethyl)benzoyl]-2,5-diaza- bicyclo[2.2.1]hept-2-yl}eth- yl)phenoxy]butanenitrile | 486 |
| 58 | 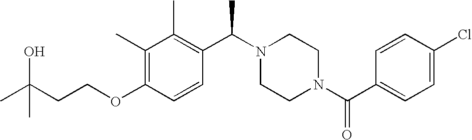 | 4-(4-{(1R)-1-[4-(4-chloro- benzoyl)piperazin-1-yl]ethyl}-2,3-di- methylphenoxy)-2-methylbutan-2-ol | 459 |
| 59 | 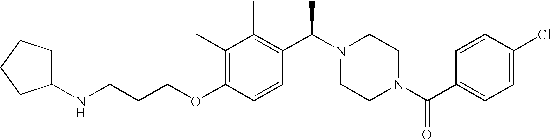 | N-[3-(4-{(1R)-1-[4-(4-chloro- benzoyl)piperazin-1-yl]ethyl}-2,3-di- methylphenoxy)-pro- pyl]cyclopentanamine | 498 |
| 60 | 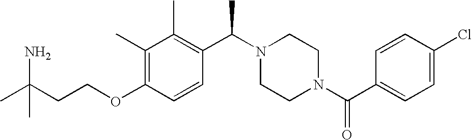 | 4-(4-{(1R)-1-[4-(4-chloro- benzoyl)piperazin-1-yl]ethyl}-2,3-di- methylphenoxy)-2-methylbutan-2-amine | 458 |
| 61 | 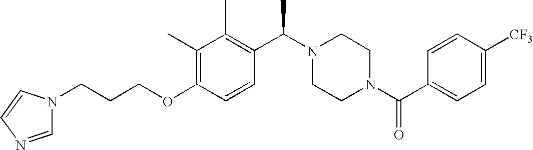 | 1-((1R)-1-{4-[3-(1H-imidazol-1-yl)pro- poxy]-2,3-di- methylphenyl}ethyl)-4-[4-(tri- fluoromethyl)benzoyl]piperazine | 515 |
| 62 | 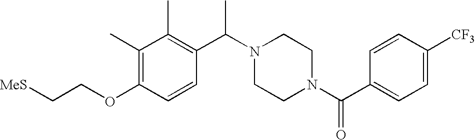 | 1-(1-{2,3-dimethyl-4-[2-(meth- ylthio)ethoxy]phenyl}ethyl)-4-[4-(tri- fluoromethyl)-ben- zoyl]piperazine | 481 |
| 63 | 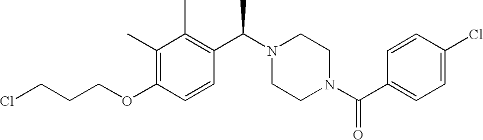 | 1-(4-chlorobenzoyl)-4-{(1R)-1-[4-(3-chloro- propoxy)-2,3-di- methylphenyl]ethyl}piperazine | 449 |
| 64 | 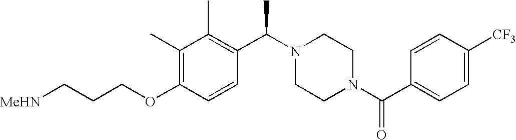 | 3-[2,3-dimethyl-4-((1R)-1-{4-[4-(tri- fluoromethyl)benzoyl]piperazin-1-yl}eth- yl)phenoxy]-N-methylpropan-1-amine | 478 |
| 65 | 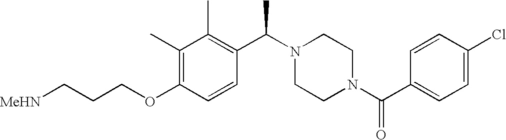 | 3-(4-{(1R)-1-[4-(4-chlorobenzoyl)-pipe- razin-1-yl]ethyl}-2,3-dimethyl-phe- noxy)-N-methylpropan-1-amine | 444 |
| 66 | 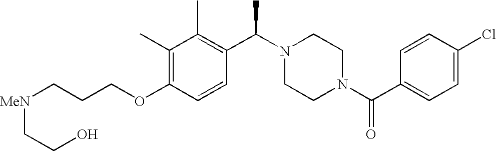 | (4-Chloro-phenyl)-{4-[(R)-1-(4-{3-[(2-hy- droxy-ethyl)-methyl-amino]-pro- poxy}-2,3-dimethyl-phenyl)-ethyl]-pipe- razin-1-yl}-methanone | 488 |
| 67 | 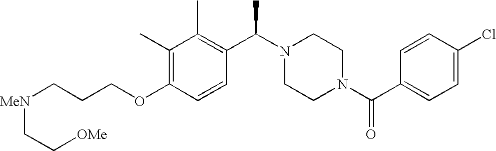 | (4-Chloro-phenyl)-{4-[(R)-1-(4-{3-[(2-meth- oxy-ethyl)-methyl-amino]-pro- poxy}-2,3-dimethyl-phenyl)-ethyl]-pipe- razin-1-yl}-methanone | 502 |
| 68 | 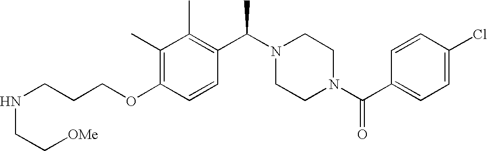 | (4-Chloro-phenyl)-[4-((R)-1-{4-[3-(2-meth- oxy-ethylamino)-propoxy]-2,3-di- methyl-phenyl}-ethyl)-piperazin-1-yl]-meth- anone | 488 |
| 69 | 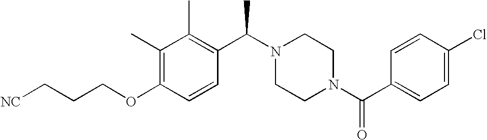 | 4-(4-{(1R)-1-[4-(4-chloro- benzoyl)piperazin-1-yl]ethyl}-2,3-di- methyl-phenoxy)butanenitrile | 440 |
| 70 | 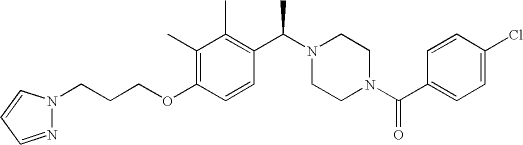 | 1-(4-chlorobenzoyl)-4-((1R)-1-{2,3-di- methyl-4-[3-(1H-pyrazol-1-yl)pro- poxy]phenyl}ethyl)piperazine | 481 |
| 71 | 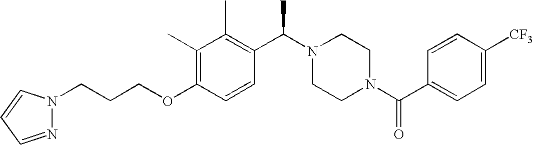 | 1-((1R)-1-{2,3-dimethyl-4-[3-(1H-py- razol-1-yl)propoxy]phenyl}-ethyl)-4-[4-(tri- fluoromethyl)-ben- zoyl]piperazine | 515 |
| 72 | 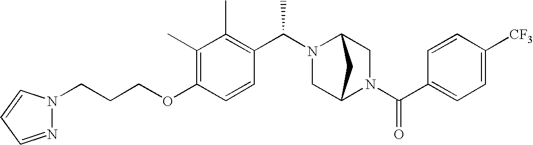 | (1S,4S)-2-((1S)-1-{2,3-dimethyl-4-[3-(1H-py- razol-1-yl)propoxy]-phe- nyl}ethyl)-5-[4-(trifluoro-meth- yl)benzoyl]-2,5-diaza- bicyclo[2.2.1]heptane | 527 |
| 73 | 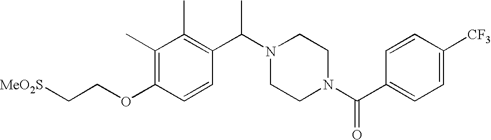 | 1-(1-{2,3-dimethyl-4-[2-(meth- ylsulfonyl)ethoxy]phenyl}ethyl)-4-[4-(tri- fluoromethyl)-benzoyl]piperazine | 513 |
| 74 | 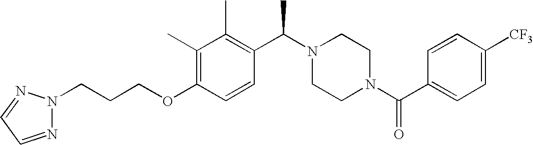 | 1-((1R)-1-{2,3-dimethyl-4-[3-(2H-1,2,3-tri- azol-2-yl)propoxy]-phe- nyl}ethyl)-4-[4-(trifluoro- methyl)benzoyl]piperazine | 516 |
| 75 | 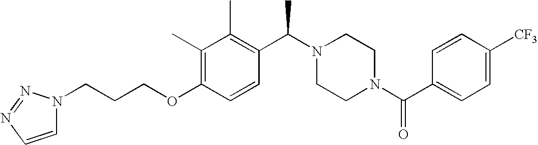 | 1-((1R)-1-{2,3-dimethyl-4-[3-(1H-1,2,3-tri- azol-1-yl)propoxy]-phe- nyl}ethyl)-4-[4-(tri- fluoromethyl)benzoyl]piperazine | 516 |
| 76 | 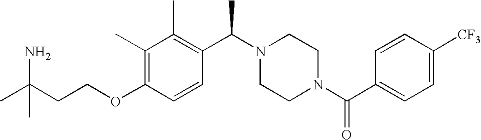 | 4-[2,3-dimethyl-4-((1R)-1-{4-[4-(tri- fluoromethyl)benzoyl]piperazin-1-yl}eth- yl)phenoxy]-2-methylbutan-2-amine | 492 |
| 77 | 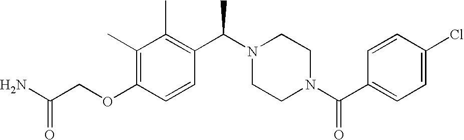 | 2-(4-{(1R)-1-[4-(4-chloro- benzoyl)piperazin-1-yl]ethyl}-2,3-di- methyl-phenoxy)acetamide | 423 |
| 78 | 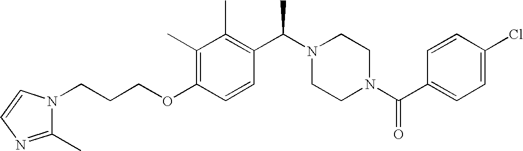 | 1-(4-chlorobenzoyl)-4-((1R)-1-{2,3-di- methyl-4-[3-(2-methyl-1H-imidazol-1-yl)pro- poxy]phenyl}-ethyl)piperazine | 495 |
| 79 | 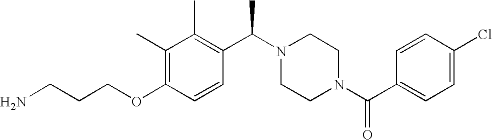 | 3-(4-{(1R)-1-[4-(4-chloro- benzoyl)piperazin-1-yl]ethyl}-2,3-di- methylphenoxy)-propan-1-amine | 430 |
| 80 | 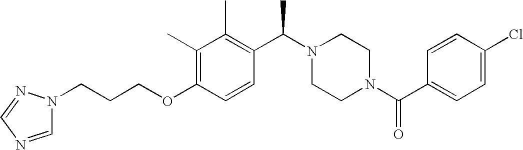 | 1-(4-chlorobenzoyl)-4-((1R)-1-{2,3-di- methyl-4-[3-(1H-1,2,4-triazol-1-yl)pro- poxy]phenyl}-ethyl)piperazine | 482 |
| 81 | 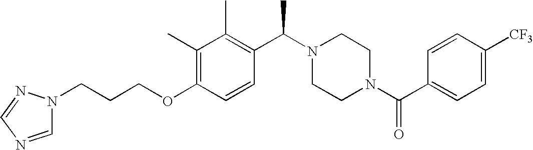 | 1-((1R)-1-{2,3-dimethyl-4-[3-(1H-1,2,4-tri- azol-1-yl)pro- poxy]phenyl}ethyl)-4-[4-(tri- fluoromethyl)benzoyl]piperazine | 516 |
| 82 | 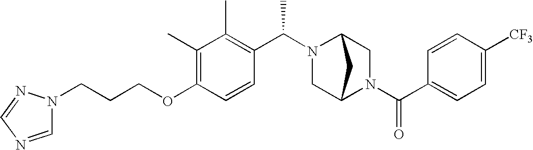 | (1S,4S)-2-((1R)-1-{2,3-dimethyl-4-[3-(1H-1,2,4-tri- azol-1-yl)pro- poxy]phenyl}ethyl)-5-[4-(tri- fluoromethyl)benzoyl]-2,5-di- azabicyclo[2.2.1]heptane | 528 |
| 83 | 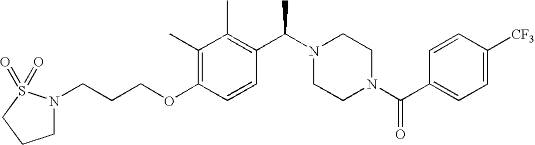 | 1-((1R)-1-{4-[3-(1,1-di- oxidoisothiazolidin-2-yl)propoxy]-2,3-di- methylphenyl}-ethyl)-4-[4-(tri- fluoromethyl)-benzoyl]piperazine | 568 |
| 84 | 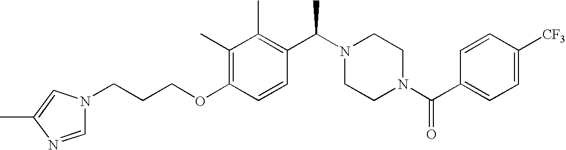 | 1-((1R)-1-{2,3-dimethyl-4-[3-(4-meth- yl-1H-imidazol-1-yl)pro- poxy]phenyl}ethyl)-4-[4-(tri- fluoromethyl)benzoyl]piperazine | 529 |
| 85 | 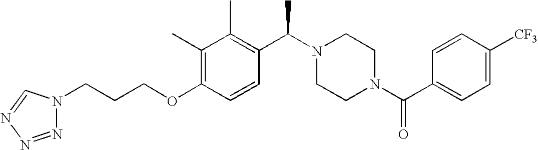 | 1-((R)-1-{2,3-dimethyl-4-[3-(1H-tetra- zol-1-yl)propoxy]phenyl}ethyl)-4-[4-(tri- fluoromethyl)benzoyl]piperazine | 517 |
| 86 | 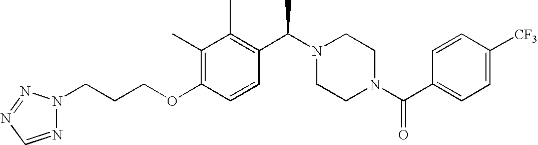 | 1-((1R)-1-{2,3-dimethyl-4-[3-(2H-tetra- zol-2-yl)propoxy]phenyl}ethyl)-4-[4-(tri- fluoromethyl)benzoyl]piperazine | 517 |
| 87 | 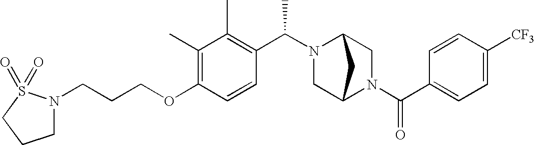 | (1S,4S)-2-((1R)-1-{4-[3-(1,1-di- oxidoisothiazolidin-2-yl)propoxy]-2,3-di- methyl-phenyl}ethyl)-5-[4-(tri- fluoro-methyl)benzoyl]-2,5-di- azabicyclo[2.2.1]heptane | 580 |
| 88 | 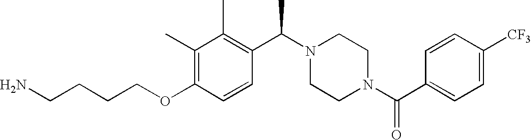 | 4-[2,3-dimethyl-4-((1R)-1-{4-[4-(tri- fluoromethyl)benzoyl]piperazin-1-yl}eth- yl)phenoxy]butan-1-amine | 478 |
| 89 | 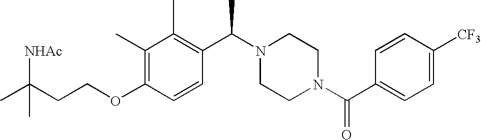 | N-{3-[2,3-dimethyl-4-((1R)-1-{4-[4-(tri- fluoromethyl)benzoyl]-piperazin-1-yl}eth- yl)phenoxy]-1,1-dimethylpropyl}acetamide | 534 |
| 90 | 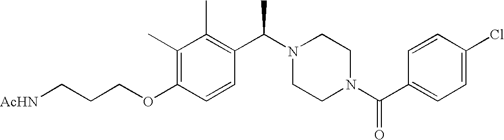 | N-[3-(4-{(1R)-1-[4-(4-chloro- benzoyl)piperazin-1-yl]ethyl}-2,3-di- methyl-phenoxy)pro- pyl]acetamide | 472 |
| 91 | 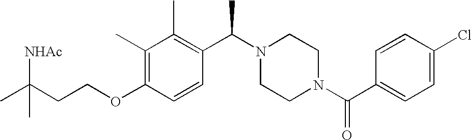 | N-[3-(4-{(1R)-1-[4-(4-chloro- benzoyl)piperazin-1-yl]ethyl}-2,3-di- methylphenoxy)-1,1-di- methylpropyl]acetamide | 500 |
| 92 | 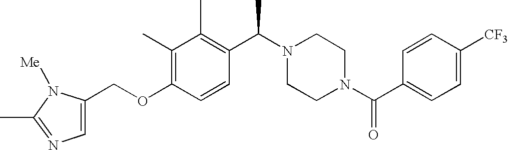 | 1-((1R)-1-{2,3-dimethyl-4-[(1-methyl-1H-imi- dazol-5-yl)meth- oxy]phenyl}ethyl)-4-[4-(tri- fluoromethyl)benzoyl]piperazine | 501 |
| 93 | 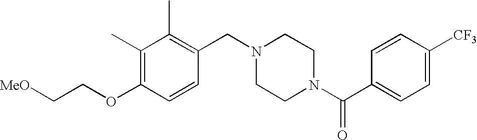 | 1-[4-(2-methoxyethoxy)-2,3-di- methylbenzyl]-4-[4-(tri- fluoromethyl)benzoyl]piperazine | 451 |
| 94 | 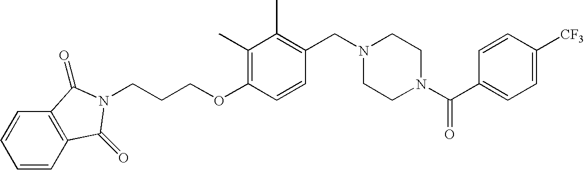 | 2-{3-[2,3-dimethyl-4-({4-[4-(tri- fluoromethyl)benzoyl]piperazin-1-yl}meth- yl)phenoxy]propyl}-1H-iso- indole-1,3(2H)-dione | 580 |
| 95 | 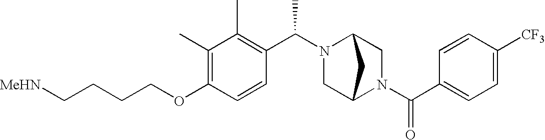 | 4-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]-N-methylbutan-1-amine | 504 |
| 96 | 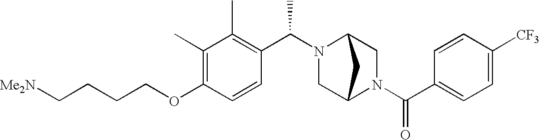 | 4-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]-N,N-di- methylbutan-1-amine | 518 |
| 97 | 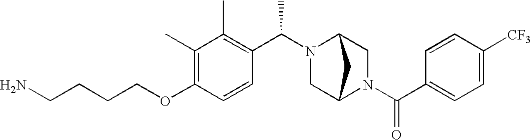 | 4-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]butan-1-amine | 490 |
| 98 | 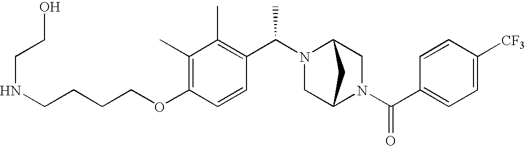 | [(1S,4S)-5-((S)-1-{4-[4-(2-Hydroxy-eth- ylamino)-butoxy]-2,3-dimethyl-phe- nyl}-ethyl)-2,5-diaza-bi- cyclo[2.2.1]hept-2-yl]-(4-tri- fluoromethyl-phenyl)-methanone | 534 |
| 99 | 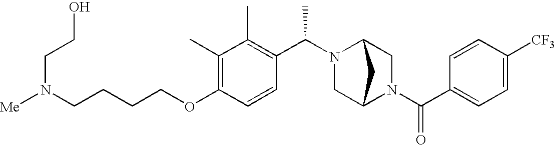 | {(1S,4S)-5-[(S)-1-(4-{4-[(2-Hydroxy-eth- yl)-methyl-amino]-butoxy}-2,3-di- methyl-phenyl)-ethyl]-2,5-diaza-bi- cyclo[2.2.1]hept-2-yl}-(4-tri- fluoromethyl-phenyl)-methanone | 548 |
| 100 | 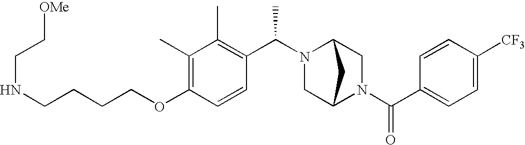 | [(2S,4S)-5-((S)-1-{4-[4-(2-Methoxy-eth- ylamino)-butoxy]-2,3-dimethyl-phe- nyl}-ethyl)-2,5-diaza-bi- cyclo[2.2.1]hept-2-yl]-(4-tri- fluoromethyl-phenyl)-methanone | 548 |
| 101 | 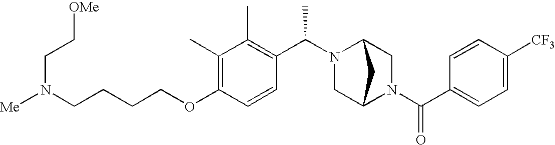 | {(1S,4S)-5-[(S)-1-(4-{4-[(2-Methoxy-eth- yl)-methyl-amino]-butoxy}-2,3-di- methyl-phenyl)-ethyl]-2,5-diaza-bi- cyclo[2.2.1]hept-2-yl}-(4-tri- fluoromethyl-phenyl)-methanone | 562 |
| 102 | 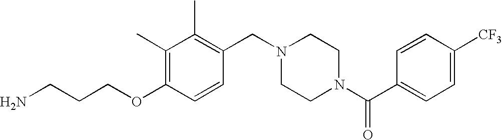 | 3-[2,3-dimethyl-4-({4-[4-(tri- fluoromethyl)benzoyl]piperazin-1-yl}meth- yl)phenoxy]propan-1-amine | 450 |
| 103 | 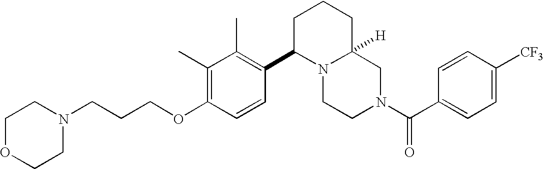 | 6-[2,3-dimethyl-4-(3-morpholin-4-yl- propoxy)phenyl]-2-[4-(tri- fluoromethyl)benzoyl]octahydro-2H-py- rido[1,2-a]pyrazine | 560 |
| 104 | 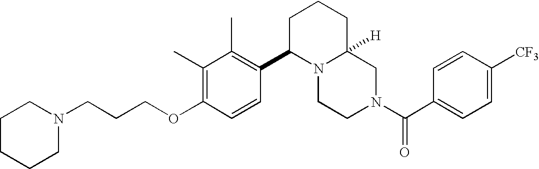 | 6-[2,3-dimethyl-4-(3-piperidin-1-yl- propoxy)phenyl]-2-[4-(tri- fluoromethyl)benzoyl]octahydro-2H-py- rido[1,2-a]pyrazine | 558 |
| 105 | 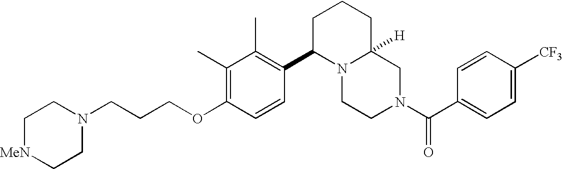 | 6-{2,3-dimethyl-4-[3-(4-meth- ylpiperazin-1-yl)pro- poxy]phenyl}-2-[4-(tri- fluoromethyl)benzoyl]octahydro-2H-py- rido[1,2-a]pyrazine | 573 |
| 106 | 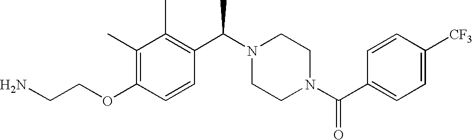 | 2-[2,3-dimethyl-4-((1R)-1-{4-[4-(tri- fluoromethyl)benzoyl]piperazin-1-yl}eth- yl)phenoxy]ethanamine | 450 |
| 107 | 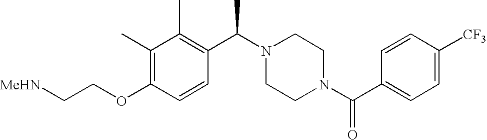 | 2-[2,3-dimethyl-4-((1R)-1-{4-[4-(tri- fluoromethyl)benzoyl]piperazin-1-yl}eth- yl)phenoxy]-N-meth- ylethanamine | 464 |
| 108 | 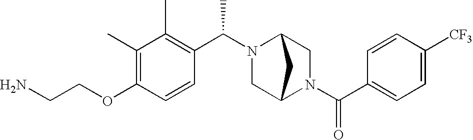 | 2-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]ethanamine | 462 |
| 109 | 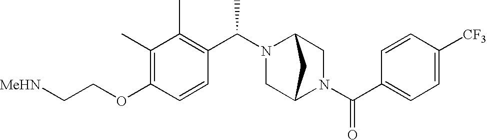 | 2-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]-N-methyl- ethanamine | 476 |
| 110 | 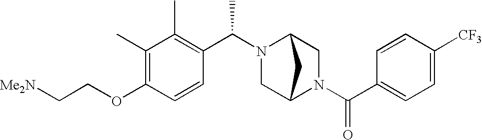 | 2-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]-N,N-di- methylethanamine | 490 |
| 111 | 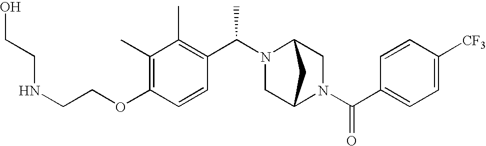 | [(1S,4S)-5-((S)-1-{4-[2-(2-Hydroxy-eth- ylamino)-ethoxy]-2,3-dimethyl-phe- nyl}-ethyl)-2,5-diaza-bicyclo-[2.2.1]hept-2-yl]-(4-tri- fluoromethyl-phe- nyl)-methanone | 506 |
| 112 | 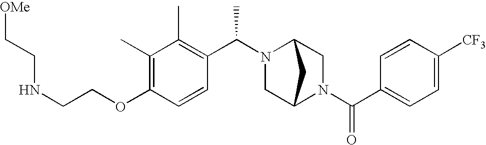 | [(1S,4S)-5-((S)-1-{4-[2-(2-Methoxy-eth- ylamino)-ethoxy]-2,3-dimethyl-phe- nyl}-ethyl)-2,5-diaza-bi- cyclo[2.2.1]hept-2-yl]-(4-tri- fluoromethyl-phenyl)-methanone | 520 |
| 113 | 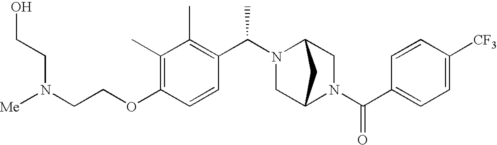 | {(1S,4S)-5-[(S)-1-(4-{2-[(2-Hydroxy-eth- yl)-methyl-amino]-ethoxy}-2,3-di- methyl-phenyl)-ethyl]-2,5-diaza-bi- cyclo[2.2.1]hept-2-yl}-(4-tri- fluoromethyl-phenyl)-methanone | 520 |
| 114 | 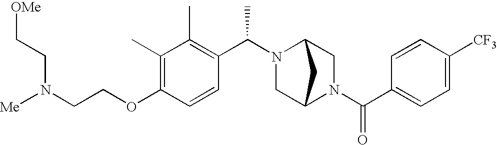 | {(1S,4S)-5-[(S)-1-(4-{2-[(2-Methoxy-eth- yl)-methyl-amino]-ethoxy}-2,3-di- methyl-phenyl)-ethyl]-2,5-diaza-bi- cyclo[2.2.1]hept-2-yl}-(4-trifluoro methyl-phenyl)-methanone | 534 |
| 115 | 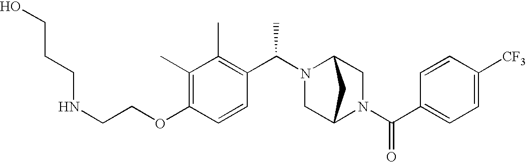 | [(1S,4S)-5-((S)-1-{4-[2-(3-Hydroxy-pro- pylamino)-ethoxy]-2,3-dimethyl-phe- nyl}-ethyl)-2,5-diaza-bi- cyclo[2.2.1]hept-2-yl]-(4-tri- fluoromethyl-phenyl)-methanone | 520 |
| 116 | 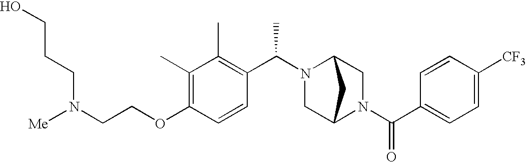 | {(1S,4S)-5-[(S)-1-(4-{2-[(3-Hydroxy-pro- pyl)-methyl-amino]-ethoxy}-2,3-di- methyl-phenyl)-ethyl]-2,5-diaza-bi- cyclo[2.2.1]hept-2-yl}-(4-tri- fluoromethyl-phenyl)-methanone | 534 |
| 117 | 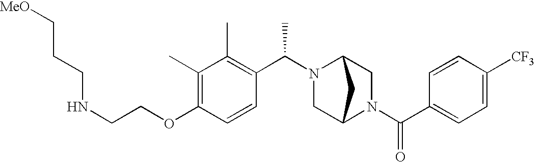 | [(1S,4S)-5-((S)-1-{4-[2-(3-Methoxy-pro- pylamino)-ethoxy]-2,3-dimethyl-phe- nyl}-ethyl)-2,5-diaza-bi- cyclo[2.2.1]hept-2-yl]-(4-tri- fluoromethyl-phenyl)-methanone | 534 |
| 118 | 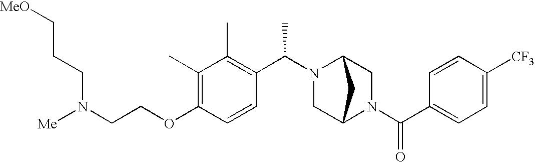 | {(1S,4S)-5-[(S)-1-(4-{2-[(3-Methoxy-pro- pyl)-methyl-amino]-ethoxy}-2,3-di- methyl-phenyl)-ethyl]-2,5-diaza-bi- cyclo[2.2.1]hept-2-yl}-(4-tri- fluoromethyl-phenyl)-methanone | 548 |
| 119 | 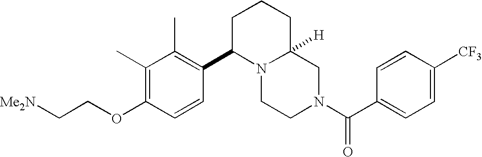 | 2-(2,3-dimethyl-4-{2-[4-(tri- fluoromethyl)benzoyl]octahydro-2H-py- rido[1,2-a]pyrazin-6-yl}phe- noxy)-N,N-dimethylethanamine | 504 |
| 120 | 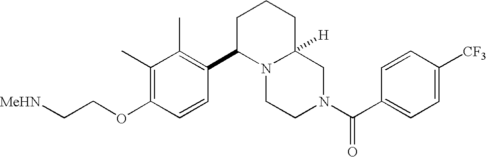 | {(6R,9aS)-6-[2,3-Dimethyl-4-(2-meth- ylamino-ethoxy)-phenyl]-octa- hydro-pyrido[1,2-a]pyrazin-2-yl}-(4-tri- fluoromethyl-phenyl)-methanone | 490 |
| 121 | 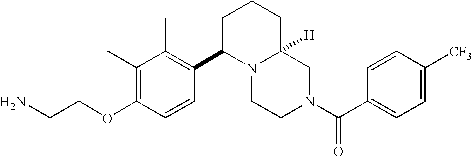 | {(6R,9aS)-6-[4-(2-Amino-ethoxy)-2,3-di- methyl-phenyl]-octahydro-py- rido[1,2-a]pyrazin-2-yl}-(4-tri- fluoromethyl-phenyl)-methanone | 476 |
| 122 | 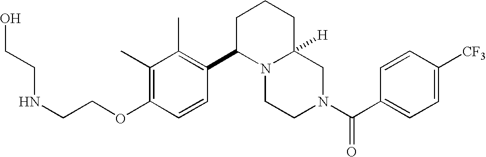 | ((6R,9aS)-6-{4-[2-(2-Hydroxy-eth- ylamino)-ethoxy]-2,3-dimethyl-phe- nyl}-octahydro-pyrido[1,2-a]py- razin-2-yl)-(4-trifluoromethyl-phe- nyl)-methanone | 520 |
| 123 | 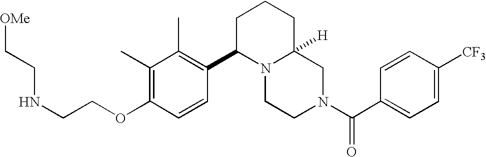 | ((6R,9aS)-6-{4-[2-(2-Methoxy-eth- ylamino)-ethoxy]-2,3-dimethyl-phe- nyl}-octahydro-pyrido[1,2-a]py- razin-2-yl)-(4-trifluoromethyl-phe- nyl)-methanone | 534 |
| 124 | 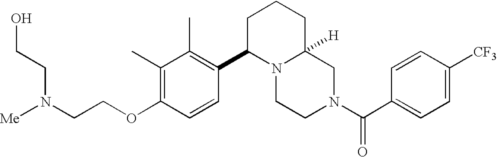 | [(6R,9aS)-6-(4-{2-[(2-Hydroxy-ethyl)-meth- yl-amino]-ethoxy}-2,3-dimethyl-phe- nyl)-octahydro-pyrido[1,2-a]py- razin-2-yl]-(4-trifluoromethyl-phe- nyl)-methanone | 534 |
| 125 | 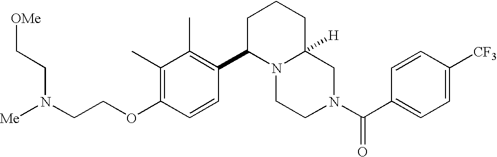 | [(6R,9aS)-6-(4-{2-[(2-Methoxy-ethyl)-meth- yl-amino]-ethoxy}-2,3-dimethyl-phe- nyl)-octahydro-pyrido[1,2-a]py- razin-2-yl]-(4-trifluoromethyl-phe- nyl)-methanone | 548 |
| 126 | 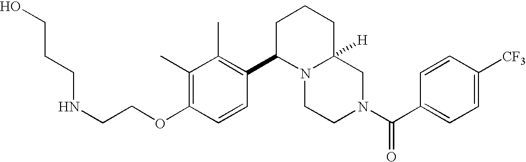 | ((6R,9aS)-6-{4-[2-(3-Hydroxy-pro- pylamino)-ethoxy]-2,3-dimethyl-phe- nyl}-octahydro-pyrido[1,2-a]py- razin-2-yl)-(4-trifluoromethyl-phe- nyl)-methanone | 534 |
| 127 | 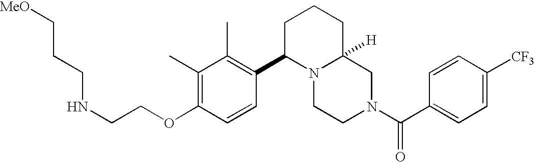 | ((6R,9aS)-6-{4-[2-(3-Methoxy-pro- pylamino)-ethoxy]-2,3-dimethyl-phe- nyl}-octahydro-pyrido[1,2-a]py- razin-2-yl)-(4-trifluoromethyl-phe- nyl)-methanone | 548 |
| 128 | 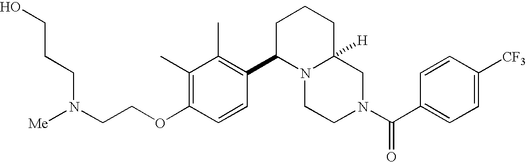 | [(6R,9aS)-6-(4-{2-[(3-Hydroxy-pro- pyl)-methyl-amino]-ethoxy}-2,3-di- methyl-phenyl)-octahydro-py- rido[1,2-a]pyrazin-2-yl]-(4-tri- fluoromethyl-phenyl)-methanone | 548 |
| 129 | 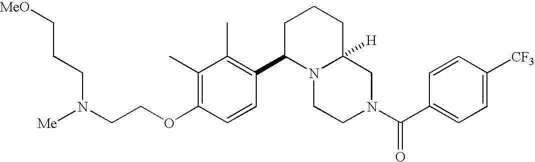 | [(6R,9aS)-6-(4-{2-[(3-Methoxy-pro- pyl)-methyl-amino]-ethoxy}-2,3-di- methyl-phenyl)-octahydro-py- rido[1,2-a]pyrazin-2-yl]-(4-tri- fluoromethyl-phenyl)-methanone | 562 |
| 130 | 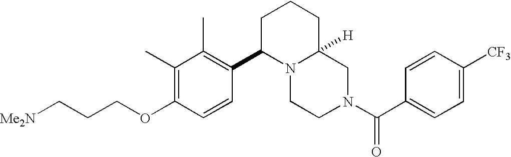 | {(6R,9aS)-6-[4-(3-Dimethylamino-pro- poxy)-2,3-dimethyl-phenyl]-octa- hydro-pyrido[1,2-a]pyrazin-2-yl}-(4-tri- fluoromethyl-phenyl)-methanone | 518 |
| 131 | 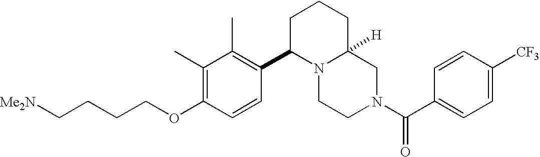 | 4-(2,3-dimethyl-4-{2-[4-(tri- fluoromethyl)benzoyl]octahydro-2H-py- rido[1,2-a]pyrazin-6-yl}phe- noxy)-N,N-dimethylbutan-1-amine | 532 |
| 132 | 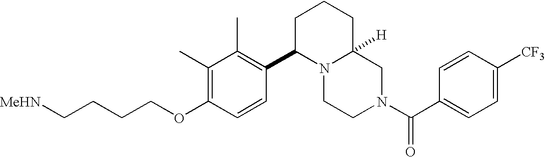 | {(6R,9aS)-6-[2,3-Dimethyl-4-(4-meth- ylamino-butoxy)-phenyl]-octa- hydro-pyrido[1,2-a]pyrazin-2-yl}-(4-tri- fluoromethyl-phenyl)-methanone | 518 |
| 133 | 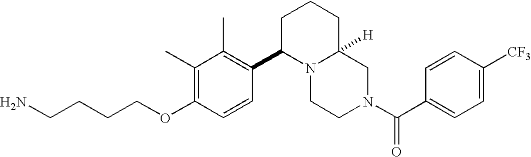 | {(6R,9aS)-6-[4-(4-Amino-butoxy)-2,3-di- methyl-phenyl]-octahydro-py- rido[1,2-a]pyrazin-2-yl}-(4-tri- fluoromethyl-phenyl)-methanone | 504 |
| 134 | 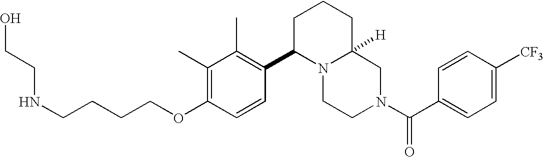 | ((6R,9aS)-6-{4-[4-(2-Hydroxy-eth- ylamino)-butoxy]-2,3-dimethyl-phe- nyl}-octahydro-pyrido[1,2-a]py- razin-2-yl)-(4-trifluoromethyl-phe- nyl)-methanone | 548 |
| 135 | 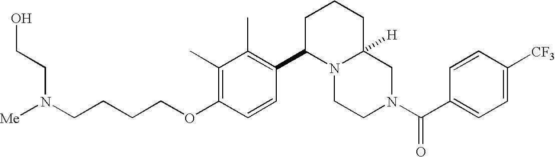 | [(6R,9aS)-6-(4-{4-[(2-Hydroxy-ethyl)-meth- yl-amino]-butoxy}-2,3-dimethyl-phe- nyl)-octahydro-pyrido[1,2-a]py- razin-2-yl]-(4-trifluoromethyl-phe- nyl)-methanone | 562 |
| 136 | 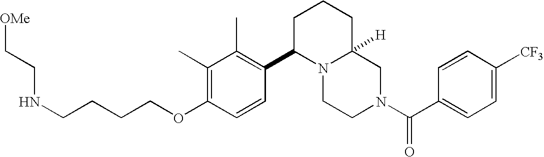 | ((6R,9aS)-6-{4-[4-(2-Methoxy-eth- ylamino)-butoxy]-2,3-dimethyl-phe- nyl}-octahydro-pyrido[1,2-a]py- razin-2-yl)-(4-trifluoromethyl-phe- nyl)-methanone | 562 |
| 137 | 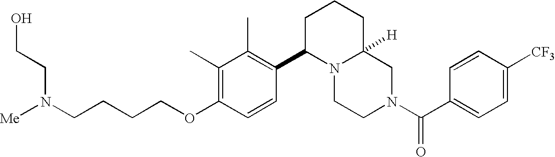 | [(6R,9aS)-6-(4-{4-[(2-Methoxy-ethyl)-meth- yl-amino]-butoxy}-2,3-dimethyl-phe- nyl)-octahydro-pyrido[1,2-a]py- razin-2-yl]-(4-trifluoromethyl-phe- nyl)-methanone | 576 |
| 138 | 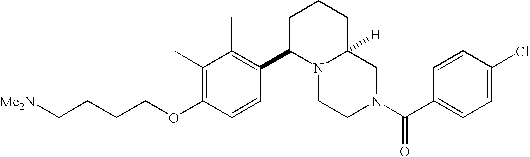 | (4-Chloro-phenyl)-{(6R,9aS)-6-[4-(4-di- methylamino-butoxy)-2,3-dimethyl-phe- nyl]-octahydro-pyrido[1,2-a]py- razin-2-yl}-methanone | 498 |
| 139 | 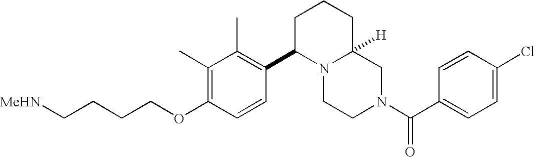 | (4-Chloro-phenyl)-{(6R,9aS)-6-[2,3-di- methyl-4-(4-methylamino-butoxy)-phe- nyl]-octahydro-pyrido[1,2-a]pyrazin-2-yl}-methanone | 484 |
| 140 | 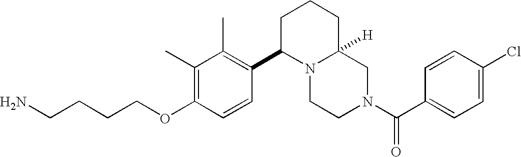 | {(6R,9aS)-6-[4-(4-Amino-butoxy)-2,3-di- methyl-phenyl]-octahydro-py- rido[1,2-a]pyrazin-2-yl}-(4-chloro-phe- nyl)-methanone | 470 |
| 141 | 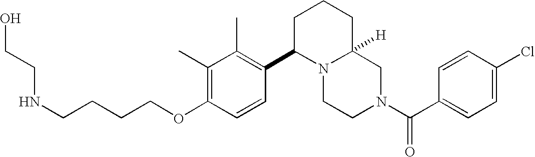 | (4-Chloro-phenyl)-((6R,9aS)-6-{4-[4-(2-hy- droxy-ethylamino)-butoxy]-2,3-di- methyl-phenyl}-octahydro-py- rido[1,2-a]pyrazin-2-yl)-methanone | 514 |
| 142 | 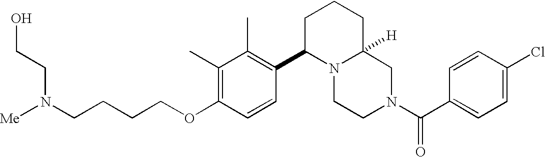 | (4-Chloro-phenyl)-[(6R,9aS)-6-(4-{4-[(2-hy- droxy-ethyl)-methyl-amino]-bu- toxy}-2,3-dimethyl-phenyl)-octa- hydro-pyrido[1,2-a]pyrazin-2-yl]-meth- anone | 528 |
| 143 | 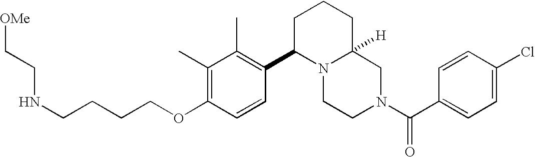 | (4-Chloro-phenyl)-((6R,9aS)-6-{4-[4-(2-meth- oxy-ethylamino)-butoxy]-2,3-di- methyl-phenyl}-octahydro-py- rido[1,2-a]pyrazin-2-yl)-methanone | 528 |
| 144 | 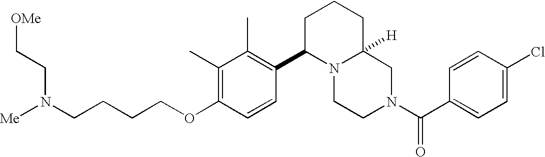 | (4-Chloro-phenyl)-[(6R,9aS)-6-(4-{4-[(2-meth- oxy-ethyl)-methyl-amino]-bu- toxy}-2,3-dimethyl-phenyl)-octa- hydro-pyrido[1,2-a]pyrazin-2-yl]-meth- anone | 542 |
| 145 | 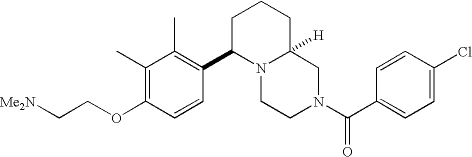 | (4-Chloro-phenyl)-{(6R,9aS)-6-[4-(2-di- methylamino-ethoxy)-2,3-dimethyl-phe- nyl]-octahydro-pyrido[1,2-a]py- razin-2-yl}-methanone | 470 |
| 146 | 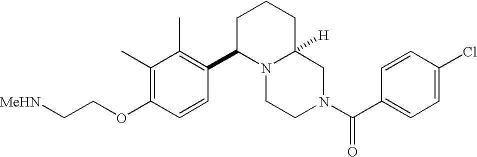 | (4-Chloro-phenyl)-{(6R,9aS)-6-[2,3-di- methyl-4-(2-methylamino-ethoxy)-phe- nyl]-octahydro-pyrido[1,2-a]py- razin-2-yl}-methanone | 456 |
| 147 | 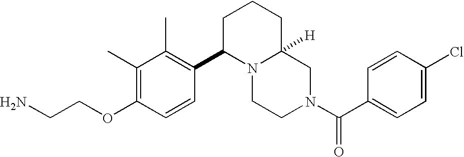 | {(6R,9aS)-6-[4-(2-Amino-ethoxy)-2,3-di- methyl-phenyl]-octahydro-py- rido[1,2-a]pyrazin-2-yl}-(4-chloro-phe- nyl)-methanone | 442 |
| 148 | 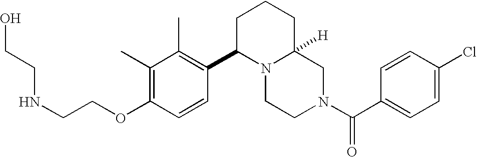 | (4-Chloro-phenyl)-((6R,9aS)-6-{4-[2-(2-hy- droxy-ethylamino)-ethoxy]-2,3-di- methyl-phenyl}-octahydro-py- rido[1,2-a]pyrazin-2-yl)-methanone | 486 |
| 149 | 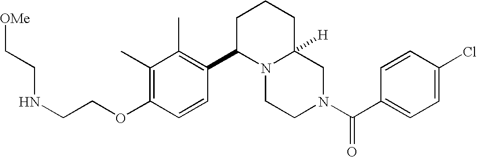 | (4-Chloro-phenyl)-((6R,9aS)-6-{4-[2-(2-meth- oxy-ethylamino)-ethoxy]-2,3-di- methyl-phenyl}-octahydro-py- rido[1,2-a]pyrazin-2-yl)-methanone | 500 |
| 150 | 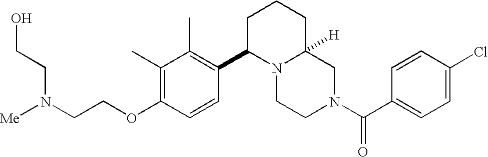 | (4-Chloro-phenyl)-[(6R,9aS)-6-(4-{2-[(2-hy- droxy-ethyl)-methyl-amino]-eth- oxy}-2,3-dimethyl-phenyl)-octa- hydro-pyrido[1,2-a]pyrazin-2-yl]-meth- anone | 500 |
| 151 | 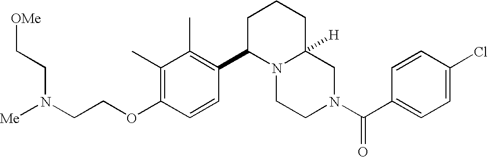 | (4-Chloro-phenyl)-[(6R,9aS)-6-(4-{2-[(2-meth- oxy-ethyl)-methyl-amino]-eth- oxy}-2,3-dimethyl-phenyl)-octa- hydro-pyrido[1,2-a]pyrazin-2-yl]-meth- anone | 514 |
| 152 | 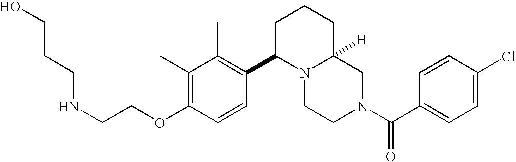 | (4-Chloro-phenyl)-((6R,9aS)-6-{4-[2-(3-hy- droxy-propylamino)-ethoxy]-2,3-di- methyl-phenyl}-octahydro-py- rido[1,2-a]pyrazin-2-yl)-methanone | 500 |
| 153 | 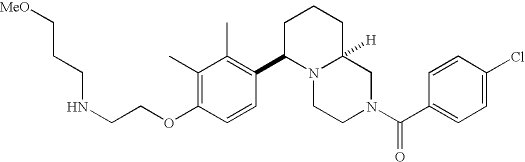 | (4-Chloro-phenyl)-((6R,9aS)-6-{4-[2-(3-meth- oxy-propylamino)-ethoxy]-2,3-di- methyl-phenyl}-octahydro-py- rido[1,2-a]pyrazin-2-yl)-methanone | 514 |
| 154 | 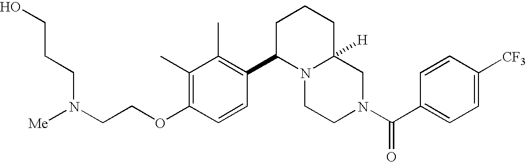 | [(6R,9aS)-6-(4-{2-[(3-Hydroxy-pro- pyl)-methyl-amino]-ethoxy}-2,3-di- methyl-phenyl)-octahydro-py- rido[1,2-a]pyrazin-2-yl]-(4-tri- fluoromethyl-phenyl)-methanone | 514 |
| 155 | 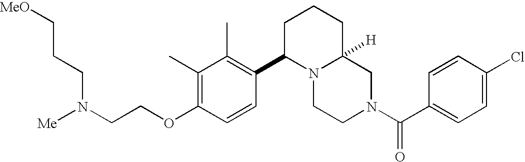 | (4-Chloro-phenyl)-[(6R,9aS)-6-(4-{2-[(3-meth- oxy-propyl)-methyl-amino]-eth- oxy}-2,3-dimethyl-phenyl)-octa- hydro-pyrido[1,2-a]pyrazin-2-yl]-meth- anone | 528 |
| 156 | 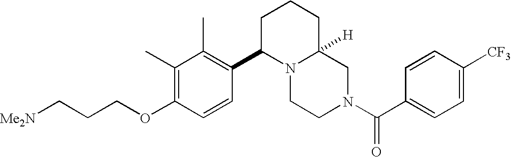 | {(6R,9aS)-6-[4-(3-Dimethylamino-pro- poxy)-2,3-dimethyl-phenyl]-octa- hydro-pyrido[1,2-a]pyrazin-2-yl}-(4-tri- fluoromethyl-phenyl)-methanone | 518 |
| 157 | 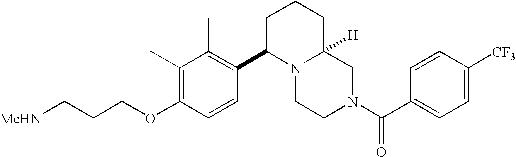 | {(6R,9aS)-6-[2,3-Dimethyl-4-(3-meth- ylamino-propoxy)-phenyl]-octa- hydro-pyrido[1,2-a]pyrazin-2-yl}-(4-tri- fluoromethyl-phenyl)-methanone | 504 |
| 158 | 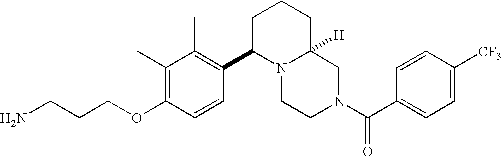 | {(6R,9aS)-6-[4-(3-Amino-propoxy)-2,3-di- methyl-phenyl]-octahydro-py- rido[1,2-a]pyrazin-2-yl}-(4-tri- fluoromethyl-phenyl)-methanone | 490 |
| 159 | 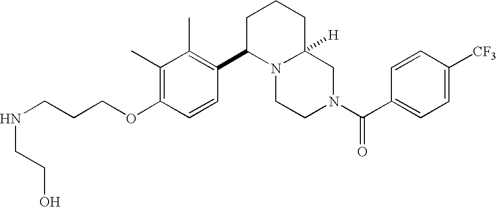 | ((6R,9aS)-6-{4-[3-(2-Hydroxy-eth- ylamino)-propoxy]-2,3-dimethyl-phe- nyl}-octahydro-pyrido[1,2-a]py- razin-2-yl)-(4-trifluoromethyl-phe- nyl)-methanone | 534 |
| 160 | 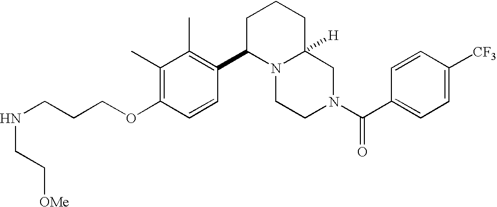 | ((6R,9aS)-6-{4-[3-(2-Methoxy-eth- ylamino)-propoxy]-2,3-dimethyl-phe- nyl}-octahydro-pyrido[1,2-a]py- razin-2-yl)-(4-trifluoromethyl-phe- nyl)-methanone | 548 |
| 161 | 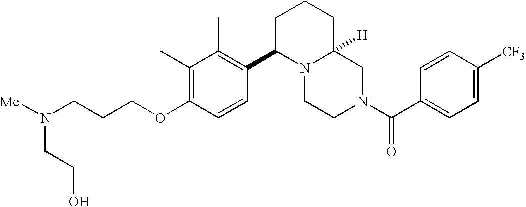 | [(6R,9aS)-6-(4-{3-[(2-Hydroxy-ethyl)-meth- yl-amino]-propoxy}-2,3-di- methyl-phenyl)-octahydro-py- rido[1,2-a]pyrazin-2-yl]-(4-tri- fluoromethyl-phenyl)-methanone | 548 |
| 162 | 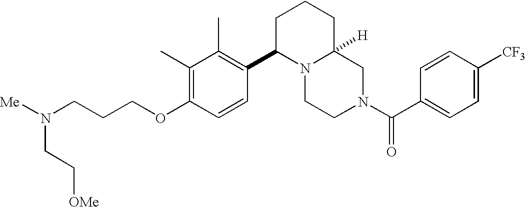 | [(6R,9aS)-6-(4-{3-[(2-Methoxy-ethyl)-meth- yl-amino]-propoxy}-2,3-di- methyl-phenyl)-octahydro-py- rido[1,2-a]pyrazin-2-yl]-(4-tri- fluoromethyl-phenyl)-methanone | 562 |
| 163 | 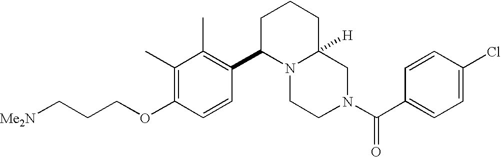 | (4-Chl,oro-phenyl)-{(6R,9aS)-6-[4-(3-di- methylamino-propoxy)-2,3-di- methyl-phenyl]-octahydro-py- rido[1,2-a]pyrazin-2-yl}-methanone | 484 |
| 164 | 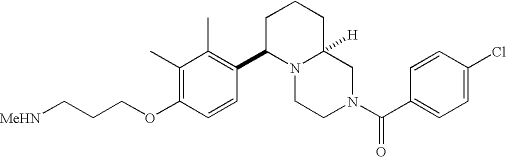 | (4-Chloro-phenyl)-{(6R,9aS)-6-[2,3-di- methyl-4-(3-methylamino-propoxy)-phe- nyl]-octahydro-pyrido[1,2-a]py- razin-2-yl}-methanone | 470 |
| 165 | 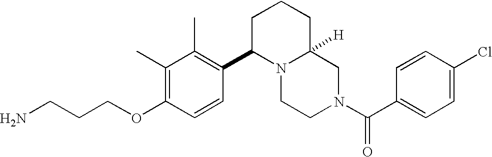 | {(6R,9aS)-6-[4-(3-Amino-propoxy)-2,3-di- methyl-phenyl]-octahydro-py- rido[1,2-a]pyrazin-2-yl}-(4-chloro-phe- nyl)-methanone | 456 |
| 166 | 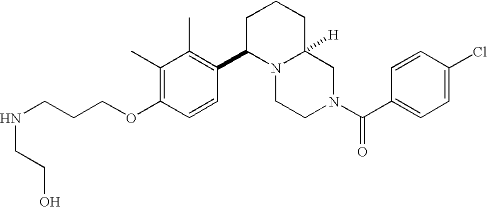 | (4-Chloro-phenyl)-((6R,9aS)-6-{4-[3-(2-hy- droxy-ethylamino)-propoxy]-2,3-di- methyl-phenyl}-octahydro-py- rido[1,2-a]pyrazin-2-yl)-methanone | 500 |
| 167 | 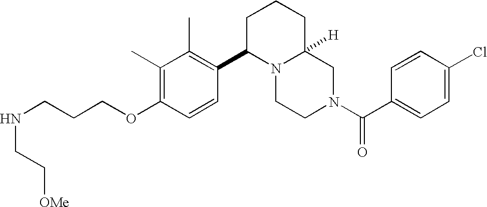 | (4-Chloro-phenyl)-((6R,9aS)-6-{4-[3-(2-meth- oxy-ethylamino)-propoxy]-2,3-di- methyl-phenyl}-octahydro-py- rido[1,2-a]pyrazin-2-yl)-methanone | 514 |
| 168 | 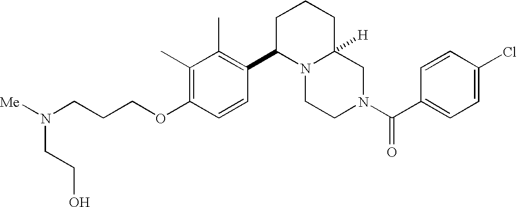 | (4-Chloro-phenyl)-[(6R,9aS)-6-(4-{3-[(2-hy- droxy-ethyl)-methyl-amino]-pro- poxy}-2,3-dimethyl-phenyl)-octa- hydro-pyrido[1,2-a]pyrazin-2-yl]-meth- anone | 514 |
| 169 | 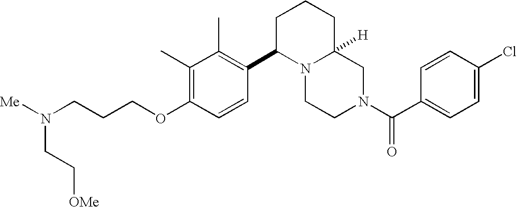 | (4-Chloro-phenyl)-[(6R,9aS)-6-(4-{3-[(2-meth- oxy-ethyl)-methyl-amino]-pro- poxy}-2,3-dimethyl-phenyl)-octa- hydro-pyrido[1,2-a]pyrazin-2-yl]-meth- anone | 528 |
| 170 | 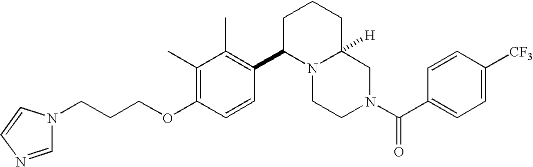 | 6-{4-[3-(1H-imidazol-1-yl)-propoxy]-2,3-di- methylphenyl}-2-[4-(tri- fluoromethyl)benzyl]octa-hydro-2H-py- rido[1,2-a]pyrazine | 541 |
| 171 | 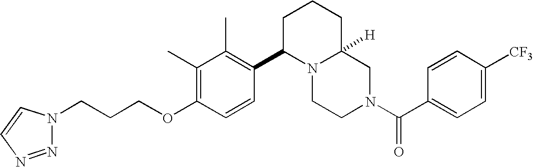 | 6-{2,3-dimethyl-4-[3-(1H-1,2,3-tri- azol-1-yl)propoxy]phenyl}-2-[4-(tri- fluoromethyl)benzoyl]octahydro-2H-py- rido[1,2-a]pyrazine | 542 |
| 172 | 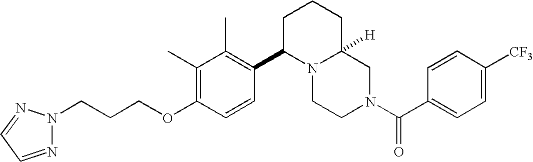 | 6-{2,3-dimethyl-4-[3-(2H-1,2,3-tri- azol-2-yl)propoxy]phenyl}-2-[4-(tri- fluoromethyl)benzoyl]octahydro-2H-py- rido[1,2-a]pyrazine | 542 |
| 173 | 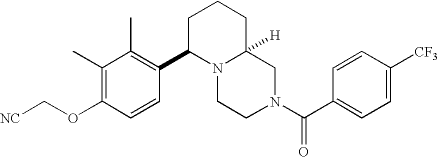 | (2,3-dimethyl-4-{2-[4-(tri- fluoromethyl)benzoyl]octahydro-2H-py- rido[1,2-a]pyrazin-6-yl}phe- noxy)acetonitrile | 472 |
| 174 | 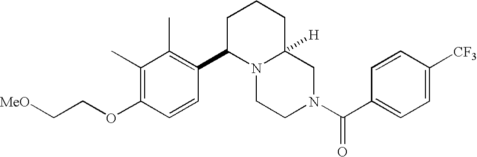 | 6-[4-(2-methoxyethoxy)-2,3-di- methylphenyl]-2-[4-(tri- fluoromethyl)benzoyl]octahydro-2H-py- rido[1,2-a]pyrazine | 491 |
| 175 | 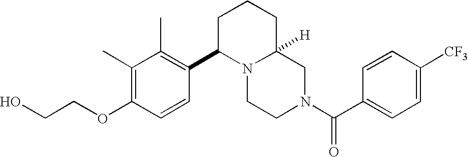 | {(6R,9aS)-6-[4-(2-Hydroxy-ethoxy)-2,3-di- methyl-phenyl]-octahydro-py- rido[1,2-a]pyrazin-2-yl}-(4-tri- fluoromethyl-phenyl)-methanone | 477 |
| 176 | 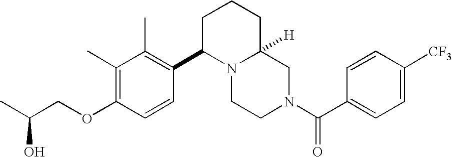 | {(6R,9aS)-6-[4-((S)-2-Hydroxy-pro- poxy)-2,3-dimethyl-phenyl]-octa- hydro-pyrido[1,2-a]pyrazin-2-yl}-(4-tri- fluoromethyl-phenyl)-methanone | 491 |
| 177 | 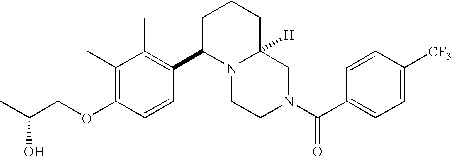 | {(6R,9aS)-6-[4-((R)-2-Hydroxy-pro- poxy)-2,3-dimethyl-phenyl]-octa- hydro-pyrido[1,2-a]pyrazin-2-yl}-(4-tri- fluoromethyl-phenyl)-methanone | 491 |
| 178 | 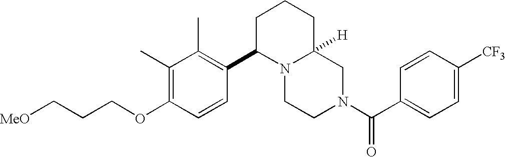 | 6-[4-(3-methoxypropoxy)-2,3-di- methylphenyl]-2-[4-(tri- fluoromethyl)benzoyl]octahydro-2H-py- rido[1,2-a]pyrazine | 505 |
| 179 | 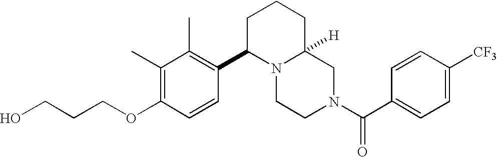 | {(6R,9aS)-6-[4-(3-Hydroxy-propoxy)-2,3-di- methyl-phenyl]-octahydro-py- rido[1,2-a]pyrazin-2-yl}-(4-tri- fluoromethyl-phenyl)-methanone | 491 |
| 180 | 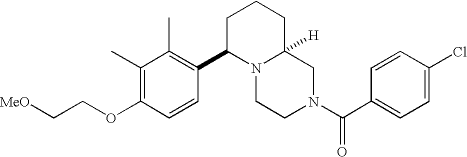 | 6-[4-(2-methoxyethoxy)-2,3-di- methylphenyl]-2-[4-chloro- benzoyl]octahydro-2H-py- rido[1,2-a]pyrazine | 457 |
| 181 | 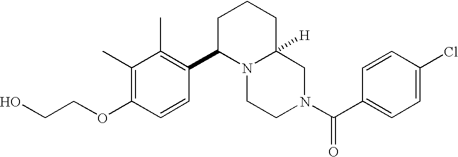 | (4-Chloro-phenyl)-{(6R,9aS)-6-[4-(2-hy- droxy-ethoxy)-2,3-dimethyl-phe- nyl]-octahydro-pyrido[1,2-a]py- razin-2-yl}-methanone | 443 |
| 182 | 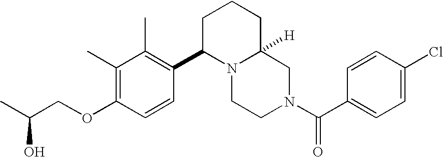 | (4-Chloro-phenyl)-{(6R,9aS)-6-[4-((S)-2-hy- droxy-propoxy)-2,3-di- methyl-phenyl]-octahydro-py- rido[1,2-a]pyrazin-2-yl}-methanone | 457 |
| 183 | 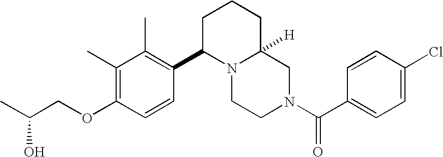 | (4-Chloro-phenyl)-{(6R,9aS)-6-[4-((R)-2-hy- droxy-propoxy)-2,3-di- methyl-phenyl]-octahydro-py- rido[1,2-a]pyrazin-2-yl}-methanone | 457 |
| 184 | 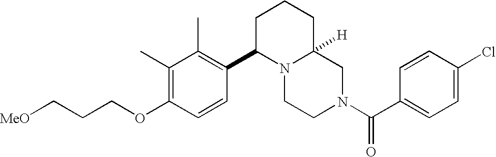 | 6-[4-(3-methoxypropoxy)-2,3-di- methylphenyl]-2-[4-chloro- benzoyl]octahydro-2H-py- rido[1,2-a]pyrazine | 471 |
| 185 | 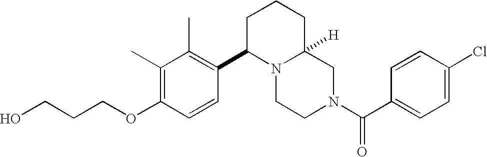 | (4-Chloro-phenyl)-{(6R,9aS)-6-[4-(3-hy- droxy-propoxy)-2,3-dimethyl-phe- nyl]-octahydro-pyrido[1,2-a]py- razin-2-yl}-methanone | 457 |
| 186 | 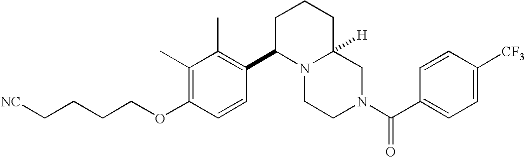 | 5-(2,3-dimethyl-4-{(6R,9aS)-2-[4-(tri- fluoromethyl)benzoyl]octa-hydro-2H-py- rido[1,2-a]pyrazin-6-yl}phe- noxy)pentanenitrile | 514 |
| 187 | 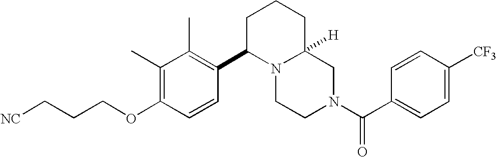 | 4-(2,3-dimethyl-4-{(6R,9aS)-2-[4-(tri- fluoromethyl)benzoyl]octahydro-2H-py- rido[1,2-a]pyrazin-6-yl}phe- noxy)butanenitrile | 500 |
| 188 | 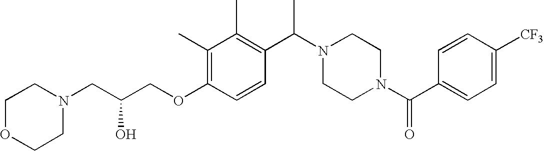 | (2R)-1-[2,3-dimethyl-4-(1-{4-[4-(tri- fluoromethyl)benzoyl]piperazin-1-yl}eth- yl)phenoxy]-3-morpholin-4-ylpro- pan-2-ol | 550 |
| 189 | 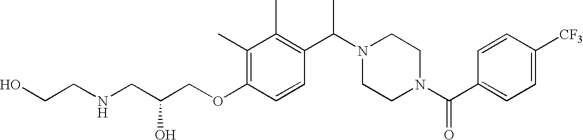 | (2R)-1-[2,3-dimethyl-4-(1-{4-[4-(tri- fluoromethyl)benzoyl]piperazin-1-yl}eth- yl)phenoxy]-3-[(2-hy- droxyethyl)amino]propan-2-ol | 538 |
| 190 | 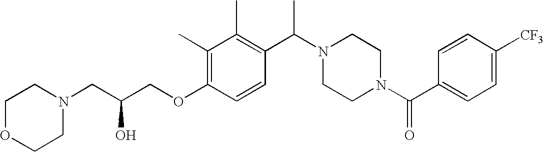 | (2S)-1-[2,3-dimethyl-4-(1-{4-[4-(tri- fluoromethyl)benzoyl]piperazin-1-yl}eth- yl)phenoxy]-3-morpholin-4-yl- propan-2-ol | 550 |
| 191 | 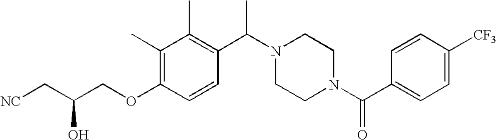 | (3S)-4-[2,3-dimethyl-4-(1-{4-[4-(tri- fluoromethyl)benzoyl]piperazin-1-yl}eth- yl)phenoxy]-3-hy- droxybutanenitrile | 490 |
| 192 | 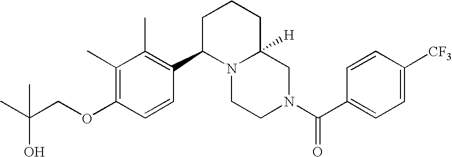 | 1-(2,3-dimethyl-4-{(6R,9aS)-2-[4-(tri- fluoromethyl)benzoyl]octa-hydro-2H-py- rido[1,2-a]pyrazin-6-yl}phe- noxy)-2-methylpropan-2-ol | 505 |
| 193 | 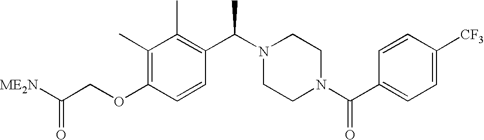 | 2-[2,3-dimethyl-4-(1-{4-[4-(tri- fluoromethyl)benzoyl]piperazin-1-yl}eth- yl)phenoxy]-N,N-di- methylacetamide | 492 |
| 194 | 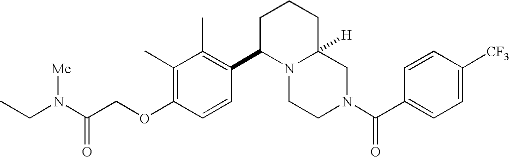 | 2-(2,3-dimethyl-4-{(6R,9aS)-2-[4-(tri- fluoromethyl)benzoyl]octahydro-2H-py- rido[1,2-a]pyrazin-6-yl}phe- noxy)-N-ethyl-N-methyl- acetamide | 532 |
| 195 | 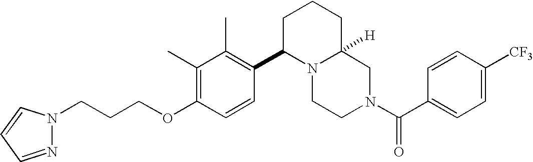 | (6R,9aS)-6-{2,3-dimethyl-4-[3-(1H-py- razol-1-yl)propoxy]phenyl}-2-[4-(tri- fluoromethyl)benzoyl]octa-hydro-2H-py- rido[1,2-a]pyrazine | 541 |
| 196 | 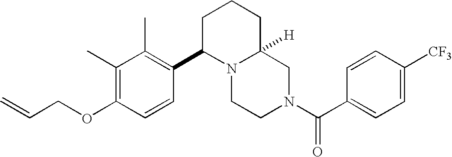 | (6R,9aS)-6-[4-(allyloxy)-2,3-di- methylphenyl]-2-[4-(trifluoro- methyl)benzoyl]octahydro-2H-py- rido[1,2-a]pyrazine | 473 |
| 197 | 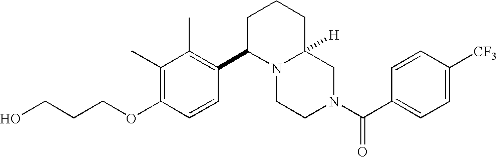 | 3-(2,3-dimethyl-4-{(6R,9aS)-2-[4-(tri- fluoromethyl)benzoyl]octa-hydro-2H-py- rido[1,2-a]pyrazin-6-yl}phe- noxy)propan-1-ol | 491 |
| 198 | 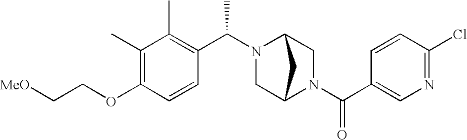 | (1S,4S)-2-[(6-chloropyridin-3-yl)car- bonyl]-5-{(1S)-1-[4-(2-meth- oxyethoxy)-2,3-di- methylphenyl]ethyl}-2,5-di- azabicyclo[2.2.1]heptane | 444 |
| 199 | 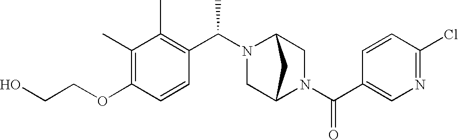 | (6-Chloro-pyridin-3-yl)-((1S,4S)-5-{(S)-1-[4-(2-hy- droxy-ethoxy)-2,3-di- methyl-phenyl]-ethyl}-2,5-diaza-bi- cyclo[2.2.1]hept-2-yl)-methanone | 430 |
| 200 | 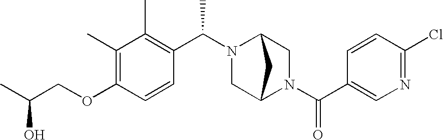 | (6-Chloro-pyridin-3-yl)-((1S,4S)-5-{(S)-1-[4-((S)-2-hy- droxy-propoxy)-2,3-di- methyl-phenyl]-ethyl}-2,5-diaza-bi- cyclo[2.2.1]hept-2-yl)-methanone | 444 |
| 201 | 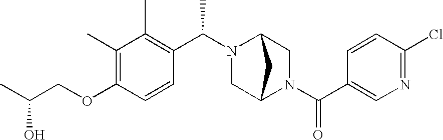 | (6-Chloro-pyridin-3-yl)-((1S,4S)-5-{(S)-1-[4-((R)-2-hy- droxy-propoxy)-2,3-di- methyl-phenyl]-ethyl}-2,5-diaza-bi- cyclo[2.2.1]hept-2-yl)-methanone | 444 |
| 202 | 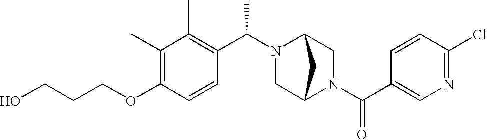 | (6-Chloro-pyridin-3-yl)-((1S,4S)-5-{(S)-1-[4-(3-hy- droxy-propoxy)-2,3-di- methyl-phenyl]-ethyl}-2,5-diaza-bi- cyclo[2.2.1]hept-2-yl)-methanone | 444 |
| 203 | 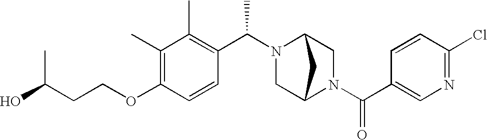 | (6-Chloro-pyridin-3-yl)-((1S,4S)-5-{(S)-1-[4-((S)-3-hy- droxy-butoxy)-2,3-di- methyl-phenyl]-ethyl}-2,5-diaza-bi- cyclo[2.2.1]hept-2-yl)-methanone | 458 |
| 204 | 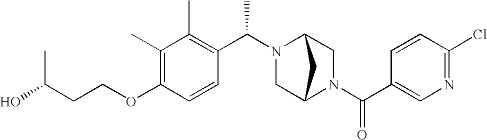 | (6-Chloro-pyridin-3-yl)-((1S,4S)-5-{(S)-1-[4-((R)-3-hy- droxy-butoxy)-2,3-di- methyl-phenyl]-ethyl}-2,5-diaza-bi- cyclo[2.2.1]hept-2-yl)-methanone | 458 |
| 205 | 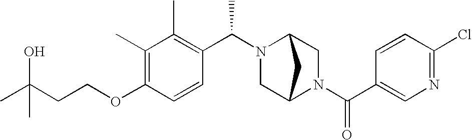 | (6-Chloro-pyridin-3-yl)-((1S,4S)-5-{(S)-1-[4-(3-hy- droxy-3-methyl-bu- boxy)-2,3-dimethyl-phenyl]-ethyl}-2,5-di- aza-bicyclo[2.2.1]hept-2-yl)-meth- anone | 472 |
| 206 | 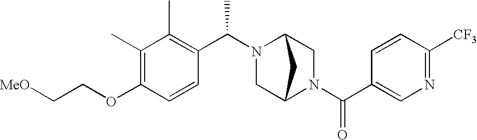 | (1S,4S)-2-{(1S)-1-[4-(2-meth- oxyethoxy)-2,3-dimethyl-phe- nyl]ethyl}-5-{[6-(trifluoro-meth- yl)pyridin-3-yl]carbonyl}-2,5-di- azabicyclo[2.2.1]heptane | 478 |
| 207 | 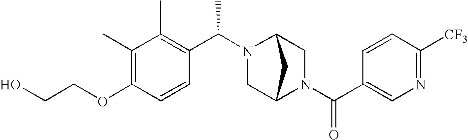 | ((1S,4S)-5-{(S)-1-[4-(2-Hydroxy-eth- oxy)-2,3-dimethyl-phenyl]-ethyl}-2,5-di- aza-bicyclo[2.2.1]hept-2-yl)-(6-tri- fluoromethyl-pyridin-3-yl)-meth- anone | 464 |
| 208 | 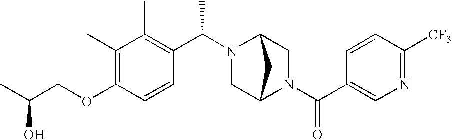 | ((1S,4S)-5-{(S)-1-[4-((S)-2-Hydroxy-pro- poxy)-2,3-dimethyl-phenyl]-ethyl}-2,5-di- aza-bicyclo[2.2.1]hept-2-yl)-(6-tri- fluoromethyl-pyridin-3-yl)-meth- anone | 478 |
| 209 | 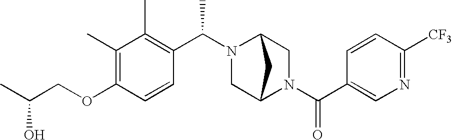 | ((1S,4S)-5-{(S)-1-[4-((R)-2-Hydroxy-pro- poxy)-2,3-dimethyl-phenyl]-ethyl}-2,5-di- aza-bicyclo[2.2.1]hept-2-yl)-(6-tri- fluoromethyl-pyridin-3-yl)-meth- anone | 478 |
| 210 | 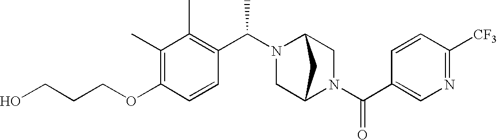 | ((1S,4S)-5-{(S)-1-[4-(3-Hydroxy-pro- poxy)-2,3-dimethyl-phenyl]-ethyl}-2,5-di- aza-bicyclo[2.2.1]hept-2-yl)-(6-tri- fluoromethyl-pyridin-3-yl)-meth- anone | 478 |
| 211 | 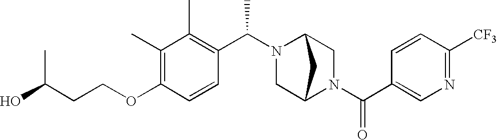 | ((1S,4S)-5-{(S)-1-[4-((S)-3-Hydroxy-bu- toxy)-2,3-dimethyl-phenyl]-ethyl}-2,5-di- aza-bicyclo[2.2.1]hept-2-yl)-(6-tri- fluoromethyl-pyridin-3-yl)-meth- anone | 492 |
| 212 | 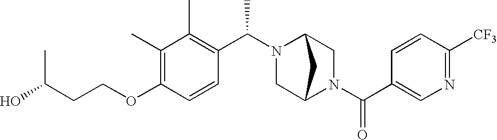 | ((1S,4S)-5-{(S)-1-[4-((R)-3-Hydroxy-bu- toxy)-2,3-dimethyl-phenyl]-ethyl}-2,5-di- aza-bicyclo[2.2.1]hept-2-yl)-(6-tri- fluoromethyl-pyridin-3-yl)-meth- anone | 492 |
| 213 | 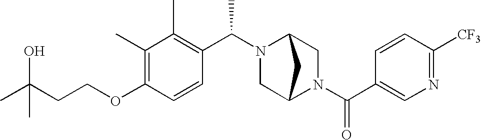 | ((1S,4S)-5-{(S)-1-[4-(3-Hydroxy-3-meth- yl-butoxy)-2,3-dimethyl-phenyl]-eth- yl}-2,5-diaza-bicyclo[2.2.1]hept-2-yl)-(6-tri- fluoromethyl-pyridin-3-yl)-meth- anone | 506 |
| 214 | 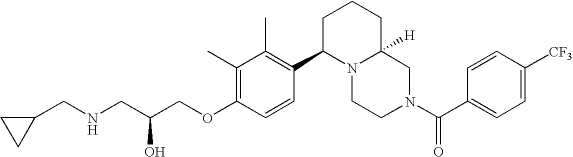 | (2S)-1-[(cyclopropylmethyl)-amino]-3-(2,3-di- methyl-4-{(6$,9aS)-2-[4-(tri- fluoromethyl)-benzoyl]octahydro-2H-py- rido[1,2-a]pyrazin-6-yl}phe- noxy)propan-2-ol | 560 |
| 215 | 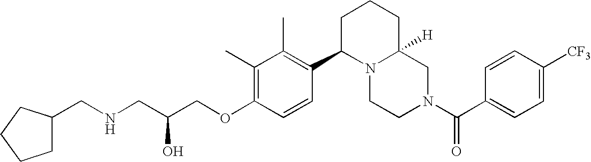 | (2S)-1-(cyclopentylamino)-3-(2,3-di- methyl-4-{(6R,9aS)-2-[4-(tri- fluoromethyl)benzoyl]octahydro-2H-py- rido[1,2-a]pyrazin-6-yl}phe- noxy)propan-2-ol | 574 |
| 216 | 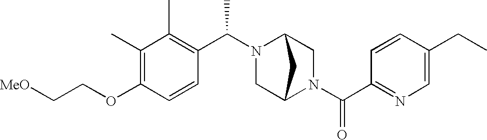 | (1S,4S)-2-[(5-ethylpyridin-2-yl)car- bonyl]-5-{(1S)-1-[4-(2-meth- oxyethoxy)-2,3-di- methylphenyl]ethyl}-2,5-di- azabicyclo[2.2.1]heptane | 438 |
| 217 | 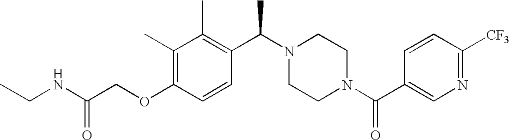 | 2-[2,3-dimethyl-4-((1R)-1-{4-[4-(tri- fluoromethyl)benzoyl]piperazin-1-yl}eth- yl)phenoxy]-N-ethylacetamide | 492 |
| 218 | 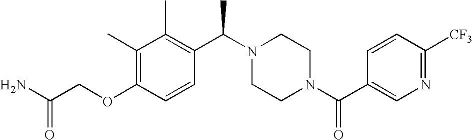 | 2-[2,3-dimethyl-4-((1R)-1-{4-[4-(tri- fluoromethyl)benzoyl]piperazin-1-yl}eth- yl)phenoxy]acetamide | 464 |
| 219 | 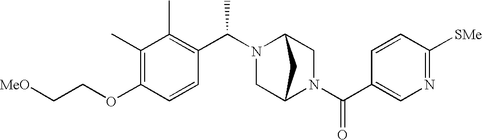 | (1S,4S)-2-{(1S)-1-[4-(2-meth- oxyethoxy)-2,3-di- methylphenyl]ethyl}-5-{[6-(meth- ylthio)pyridin-3-yl]carbonyl}-2,5-di- azabicyclo[2.2.1]heptane | 456 |
| 220 | 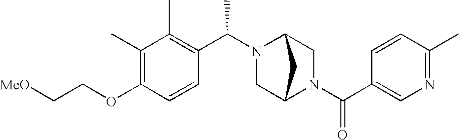 | (1S,4S)-2-{(1S)-1-[4-(2-meth- oxyethoxy)-2,3-dimethyl- phenyl]ethyl}-5-[(6-meth- ylpyridin-3-yl)carbonyl]-2,5-di- azabicyclo[2.2.1]heptane | 424 |
| 221 | 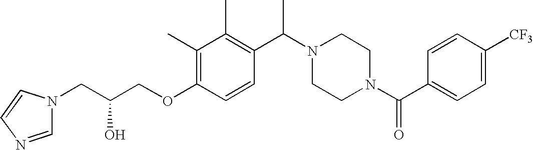 | (2R)-1-[2,3-dimethyl-4-(1-{4-[4-(tri- fluoromethyl)benzoyl]piperazin-1-yl}eth- yl)phenoxy]-3-(1H-imidazol-1-yl)pro- pan-2-ol | 531 |
| 222 | 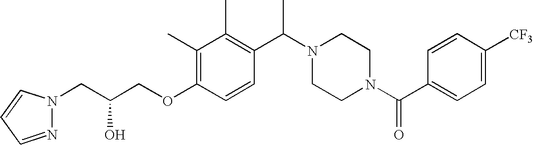 | (2R)-1-[2,3-dimethyl-4-(1-{4-[4-(tri- fluoromethyl)benzoyl]piperazin-1-yl}eth- yl)phenoxy]-3-(1H-pyrazol-1-yl)pro- pan-2-ol | 531 |
| 223 | 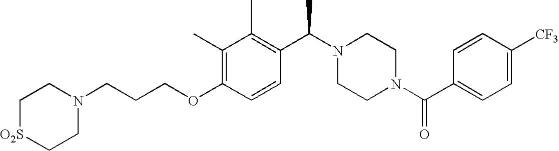 | 4-{3-[2,3-dimethyl-4-((1R)-1-{4-[4-(tri- fluoromethyl)benzoyl]piperazin-1-yl}eth- yl)phenoxy]propyl}-thio- morpholin 1,1-dioxide | 582 |
| 224 | 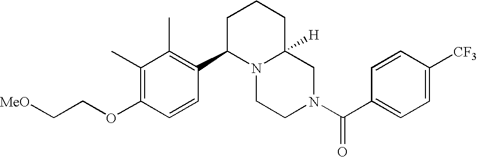 | (6R,9aS)-6-[4-(2-methoxyethoxy)-2,3-di- methylphenyl]-2-[4-(tri- fluoromethyl)benzoyl]octahydro-2H-py- rido[1,2-a]pyrazine | 491 |
| 225 | 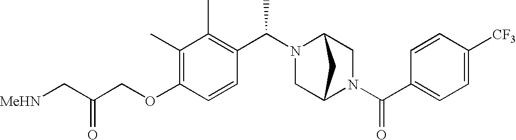 | 2-[2,3-dimethyl-4-((1R)-1-{(1R,4R)-5-[4-(tri- fluoromethyl)benzoyl]-2,5-di- azabicyclo[2.2.1]hept-2-yl}eth- yl)phenoxy]-N-methylacetamide | 490 |
| 226 | 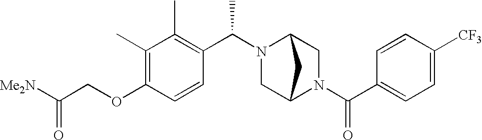 | 2-[2,3-dimethyl-4-((1R)-1-{(1R,4R)-5-[4-(tri- fluoromethyl)benzoyl]-2,5-di- azabicyclo[2.2.1]hept-2-yl}eth- yl)phenoxy]-N,N-di- methylacetamide | 504 |
| 227 | 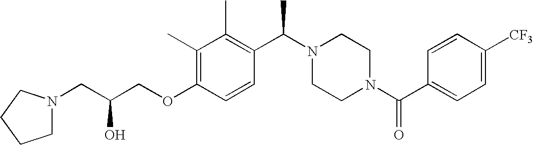 | (2S)-1-[2,3-dimethyl-4-((1R)-1-{4-[4-(tri- fluoromethyl)benzoyl]-piperazin-1-yl}eth- yl)phenoxy]-3-pyrrolidin-1-yl- propan-2-ol | 534 |
| 228 | 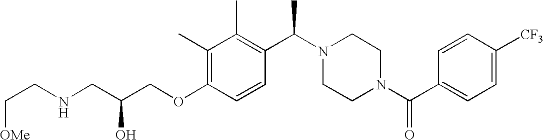 | (2S)-1-[2,3-dimethyl-4-((1R)-1-{4-[4-(tri- fluoromethyl)benzoyl]piperazin-1-yl}eth- yl)phenoxy]-3-[(2-meth- oxyethyl)amino]propan-2-ol | 538 |
| 229 | 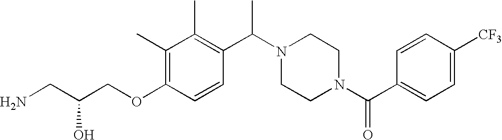 | (2R)-1-amino-3-[2,3-dimethyl-4-(1-{4-[4-(tri- fluoromethyl)benzoyl]-pipe- razin-1-yl}ethyl)-phenoxy]propan-2-ol | 480 |
| 230 | 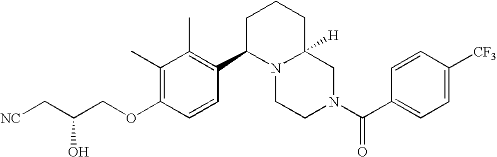 | (3R)-4-(2,3-dimethyl-4-{(6R,9aS)-2-[4-(tri- fluoromethyl)benzoyl]octa- hydro-2H-pyrido[1,2-a]pyrazin-6-yl}phe- noxy)-3-hydroxybutanenitrile | 516 |
| 231 | 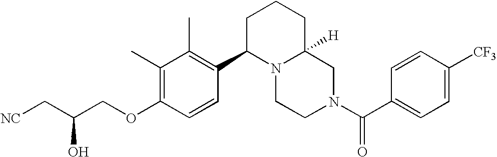 | (3S)-4-(2,3-dimethyl-4-{(6R,9aS)-2-[4-(tri- fluoromethyl)benzoyl]octa- hydro-2H-pyrido[1,2-a]pyrazin-6-yl}phe- noxy)-3-hydroxybutanenitrile | 516 |
| 232 | 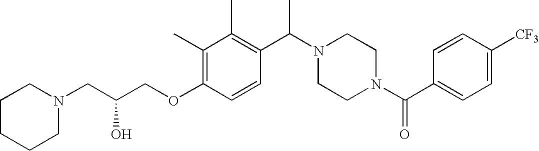 | (2R)-1-[2,3-dimethyl-4-(1-{4-[4-(tri- fluoromethyl)benzoyl]piperazin-1-yl}ethyl)phe- noxy]-3-piperidin-1-yl- propan-2-ol | 548 |
| 233 | 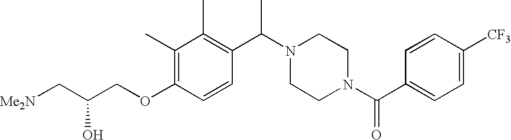 | (2R)-1-(dimethylamino)-3-[2,3-di- methyl-4-(1-{4-[4-(tri- fluoromethyl)benzoyl]piperazin-1-yl}eth- yl)phenoxy]propan-2-ol | 508 |
| 234 | 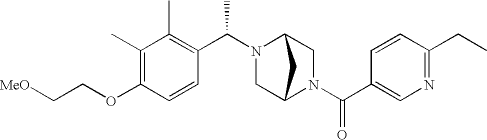 | (1S,4S)-2-[(6-ethylpyridin-3-yl)car- bonyl]-5-{(1S)-1-[4-(2-meth- oxyethoxy)-2,3-di- methylphenyl]ethyl}-2,5-di- azabicyclo[2.2.1]heptane | 438 |
| 235 | 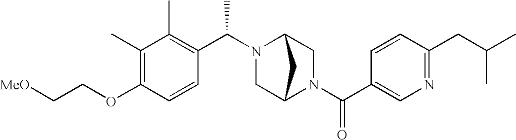 | (1S,4S)-2-[(6-isobutylpyriidn-3-yl)car- bonyl]-5-{(1S)-1-[4-(2-meth- oxyethoxy)-2,3-di- methylphenyl]ethyl}-2,5-di- azabicyclo[2.2.1]heptane | 466 |
| 236 | 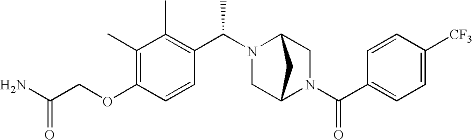 | 2-[2,3-dimethyl-4-((1R)-1-{(1R,4R)-5-[4-(tri- fluoromethyl)benzoyl]-2,5-di- azabicyclo[2.2.1]hept-2-yl}eth- yl)phenoxy]acetamide | 476 |
| 237 | 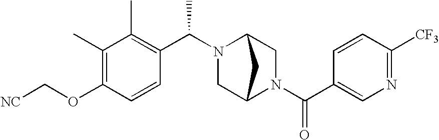 | {2,3-dimethyl-4-[(1S)-1-((1S,4S)-5-{[6-(tri- fluoromethyl)pyridin-3-yl]car- bonyl}-2,5-di- azabicyclo[2.2.1]hept-2-yl)eth- yl]phenoxy}acetonitrile | 459 |
| 238 | 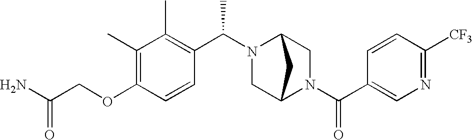 | 2-{2,3-dimethyl-4-[(1S)-1-((1S,4S)-5-{[6-tri- fluoromethyl)pyridin-3-yl]car- bonyl}-2,5-di- azabicyclo[2.2.1]hept-2-yl)eth- yl]phenoxy}acetamide | 477 |
| 239 | 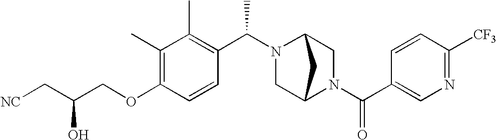 | (3S)-4-[2,3-dimethyl-4-((1S)-1-{(1S,4S)-5-[4-(tri- fluoromethyl)-ben- zoyl]-2,5-diazabicyclo-[2.2.1]hept-2-yl}eth- yl)phenoxy]-3-hy- droxybutanenitrile | 502 |
| 240 | 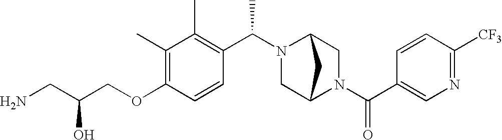 | (2S)-1-amino-3-[2,3-dimethyl-4-((1S)-1-{(1S,4S)-5-[4-(tri- fluoromethyl)benzoyl]-2,5-di- azabicyclo[2.2.1]hept-2-yl}eth- yl}phenoxy]propan-2-ol | 492 |
| 241 | 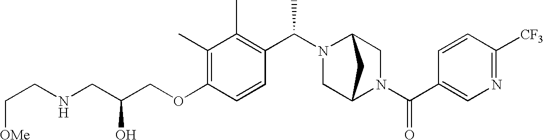 | (2S)-1-[2,3-dimethyl-4-((1S)-1-{(1S,4S)-5-[4-(tri- fluoromethyl)-ben- zoyl]-2,5-diazabicyclo-[2.2.1]hept-2-yl}eth- yl)phenoxy]-3-[(2-meth- oxyethyl)amino]propan-2-ol | 550 |
| 242 | 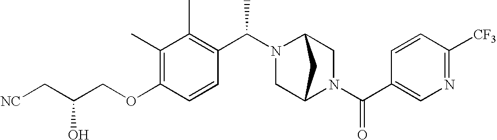 | (3R)-4-[2,3-dimethyl-4-((1S)-1-{(1S,4S)-5-[4-(tri- fluoromethyl)-ben- zoyl]-2,5-diazabicyclo[2.2.1]-hept-2-yl}eth- yl)phenoxy]-3-hydroxy- butanenitrile | 502 |
| 243 | 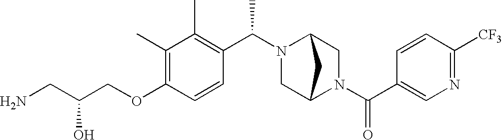 | (2R)-1-amino-3-[2,3-dimethyl-4-((1S)-1-{(1S,4S)-5-[4-(tri- fluoromethyl)benzoyl]-2,5-di- azabicyclo[2.2.1]hept-2-yl}eth- yl)phenoxy]propan-2-ol | 492 |
| 244 | 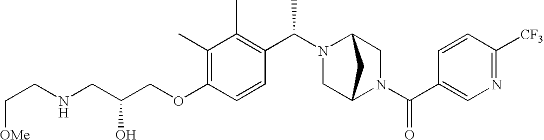 | (2R)-1-[2,3-dimethyl-4-((1S)-1-{(1S,4S)-5-[4-(tri- fluoromethyl)-ben- zoyl]-2,5-diazabicyclo[2.2.1]-hept-2-yl}eth- yl)phenoxy]-3-[(2-meth- oxyethyl)amino]propan-2-ol | 550 |
| 245 | 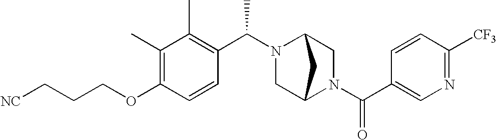 | 4-{2,3-dimethyl-4-[(1S)-1-((1S,4S)-5-{[6-(tri- fluoromethyl)pyridin-3-yl]car- bonyl}-2,5-diazabicyclo-[2.2.1]hept-2-yl)eth- yl]phenoxy}-butane- nitrile | 487 |
| 246 | 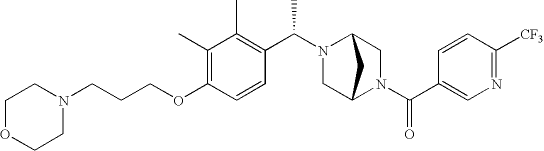 | (1S,4S)-2-{(1S)-1-[2,3-dimethyl-4-(3-mor- pholin-4-ylpropoxy)-phe- nyl]ethyl}-5-{[6-(trifluoro- methyl)pyridin-3-yl]carbonyl}-2,5-di- azabicyclo[2.2.1]heptane | 547 |
| 247 | 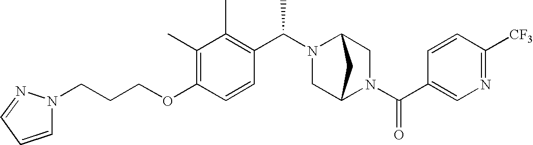 | (1S,4S)-2-((1S)-1-{2,3-dimethyl-4-[3-(1H-py- razol-1-yl)propoxy]-phe- nyl}ethyl)-5-{[6-(trifluoro- methyl)pyridin-3-yl]carbonyl}-2,5-di- azabicyclo[2.2.1]heptane | 528 |
| 248 | 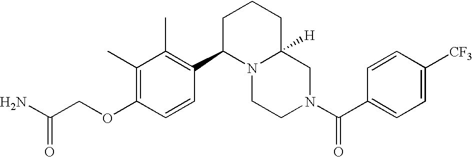 | 2-(2,3-dimethyl-4-{(6R,9aS)-2-[4-(tri- fluoromethyl)benzoyl]octahydro-2H-py- rido[1,2-a]pyrazin-6-yl}phe- noxy)acetamide | 490 |
| 249 | 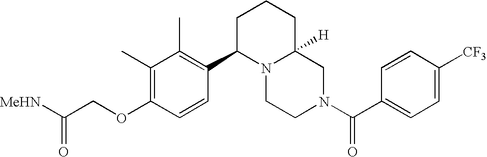 | 2-(2,3-dimethyl-4-{(6R,9aS)-2-[4-(tri- fluoromethyl)benzoyl]octahydro-2H-py- rido[1,2-a]pyrazin-6-yl}phe- noxy)-N-methylacetamide | 504 |
| 250 | 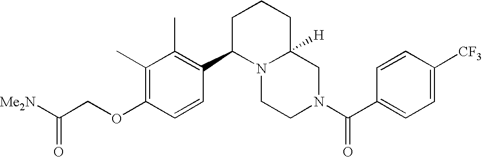 | 2-(2,3-dimethyl-4-{(6R,9aS)-2-[4-(tri- fluoromethyl)benzoyl]octahydro-2H-py- rido[1,2-a]pyrazin-6-yl}phe- noxy)-N,N-dimethylacetamide | 518 |
| 251 | 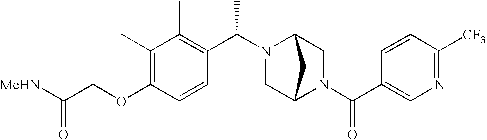 | 2-{2,3-dimethyl-4-[(1S)-1-((1S,4S)-5-{[6-(tri- fluoromethyl)pyridin-3-yl]car- bonyl}-2,5-diazabicyclo-[2.2.1]hept-2-yl)eth- yl]phenoxy}-N-meth- ylacetamide | 491 |
| 252 | 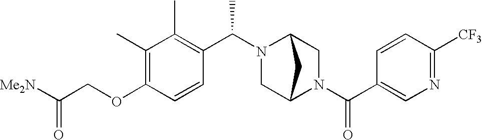 | 2-{2,3-dimethyl-4-[(1S)-1-((1S,4S)-5-{[6-(tri- fluoromethyl)pyridin-3-yl]car- bonyl}-2,5-diazabicyclo-[2.2.1]hept-2-yl)eth- yl]phenoxy}-N,N-dimethyl- acetamide | 505 |
| 253 | 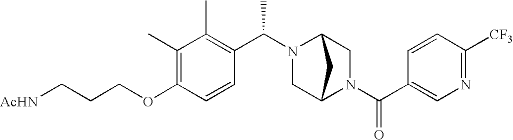 | N-(3-{2,3-dimethyl-4-[(1S)-1-((1S,4S)-5-{[6-(tri- fluoromethyl)-py- ridin-3-yl]carbonyl}-2,5-di- azabicyclo[2.2.1]hept-2-yl)eth- yl]phenoxy}propyl)acetamide | 519 |
| 254 | 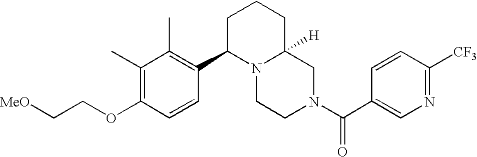 | (6R,9aS)-6-[4-(2-methoxyethoxy)-2,3-di- methylphenyl]-2-{[6-(tri- fluoromethyl)pyridin-3-yl]car- bonyl}octahydro-2H-pyrido[1,2-a]pyrazine | 492 |
| 255 | 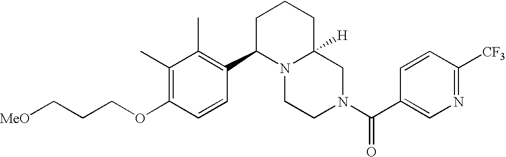 | (6R,9aS)-6-[4-(3-methoxypropoxy)-2,3-di- methylphenyl]-2-{[6-(tri- fluoromethyl)pyridin-3-yl]car- bonyl}octahydro-2H-pyrido[1,2-a]py- razine | 506 |
| 256 | 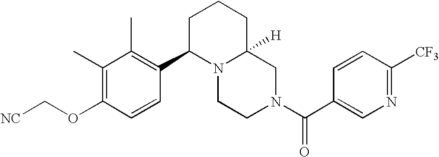 | [2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]acetonitrile | 473 |
| 257 | 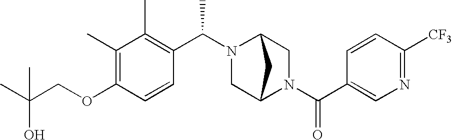 | 1-{2,3-dimethyl-4-[(1S)-1-((1S,4S)-5-{[6-(tri- fluoromethyl)pyridin-3-yl]car- bonyl}-2,5-diazabicyclo-[2.2.1]hept-2-yl)eth- yl]phenoxy}-2-methyl- propan-2-ol | 492 |
| 258 | 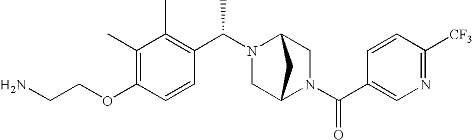 | 2-(2,3-dimethyl-4-[(1S)-1-((1S,4S)-5-{[6-(tri- fluoromethyl)pyridin-3-yl]car- bonyl}-2,5-di- azabicyclo[2.2.1]hept-2-yl)eth- yl]phenoxy}ethanamine | 463 |
| 259 | 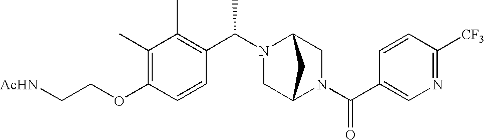 | N-(2-{2,3-dimethyl-4-[(1S)-1-((1S,4S)-5-{[6-(tri- fluoromethyl)-py- ridin-3-yl]carbonyl}-2,5-di- azabicyclo[2.2.1]hept-2-yl)ethyl]-phe- noxy}ethyl)acetamide | 505 |
| 260 | 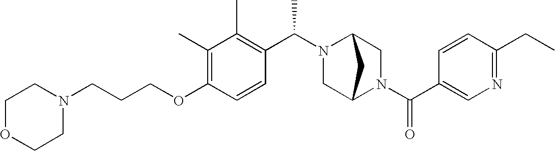 | (1S,4S)-2-{(1S)-1-[2,3-dimethyl-4-(3-mor- pholin-4-ylpropoxy)-phenyl]-eth- yl}-5-[(6-ethylpyridin-3-yl)car- bonyl]-2,5-di- azabicyclo[2.2.1]heptane | 507 |
| 261 | 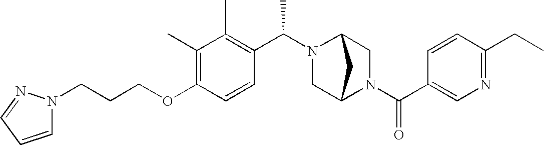 | (1S,4S)-2-((1S)-1-{2,3-dimethyl-4-[3-(1H-py- razol-1-yl)-pro- poxy]phenyl}ethyl)-5-[(6-ethyl-pyri- din-3-yl)carbonyl]-2,5-di- azabicyclo[2.2.1]heptane | 488 |
| 262 | 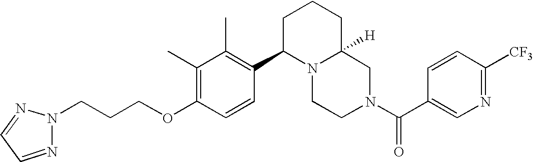 | (6R,9aS)-6-{2,3-dimethyl-4-[3-(2H-1,2,3-tri- azol-2-yl)propoxy]phenyl}-2-{[6-(tri- fluoromethyl)pyridin-3-yl]car- bonyl}octahydro-2H-pyrido[1,2-a]py- razine | 543 |
| 263 | 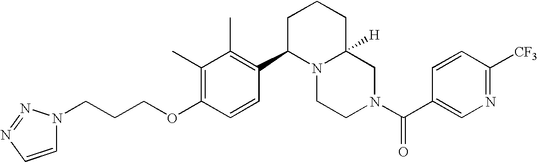 | (6R,9aS)-6-{2,3-dimethyl-4-[3-(1H-1,2,3-tri- azol-1-yl)propoxy]phenyl}-2-{[6-(tri- fluoromethyl)pyridin-3-yl]car- bonyl}octahydro-2H-pyrido[1,2-a]pyra- zine | 543 |
| 264 | 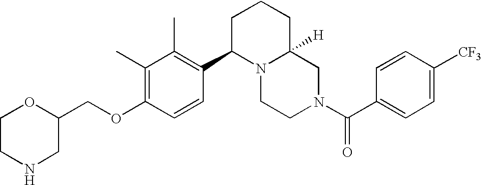 | (6R,9aS)-6-[2,3-dimethyl-4-(mor- pholin-2-ylmethoxy)phenyl]-2-[4-(tri- fluoromethyl)benzoyl]octa-hydro-2H-pyri- do[1,2-a]pyrazine | 532 |
| 265 | 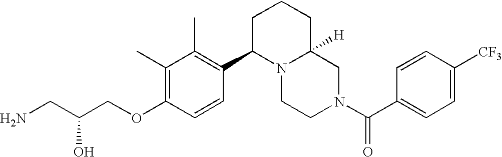 | (2R)-1-amino-3-(2,3-dimethyl-4-{(6R,9aS)-2-[4-(tri- fluoromethyl)-ben- zoyl]octahydro-2H-pyrido[1,2-a]pyra- zin-6-yl}phenoxy)propan-2-ol | 506 |
| 266 | 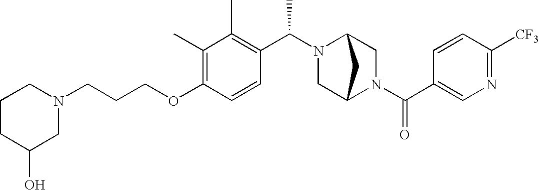 | 1-(3-{2,3-dimethyl-4-[(1S)-1-((1S,4S)-5-{[6-(tri- fluoromethyl)-pyridin-3-yl]car- bonyl}-2,5-di- azabicyclo[2.2.1]hept-2-yl)-eth- yl]phenoxy}propyl)piperidin-3-ol | 561 |
| 267 | 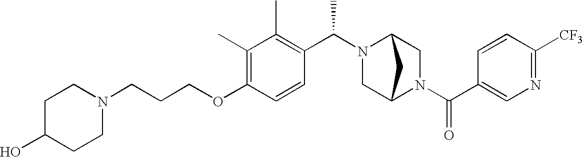 | 1-(3-{2,3-dimethyl-4-[(1S)-1-((1S,4S)-5-{[6-(tri- fluoromethyl)-pyridin-3-yl]car- bonyl}-2,5-di- azabicyclo[2.2.1]hept-2-yl)ethyl]-phe- noxy}propyl)piperidin-4-ol | 561 |
| 268 | 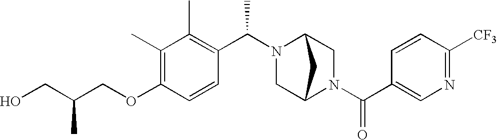 | (2S)-3-{2,3-dimethyl-4-[(1S)-1-((1S,4S)-5-{[6-(tri- fluoromethyl)-pyri- din-3-yl]carbonyl}-2,5-di- azabicyclo[2.2.1]hept-2-yl)ethyl]-phe- noxy}-2-methylpropan-1-ol | 492 |
| 269 | 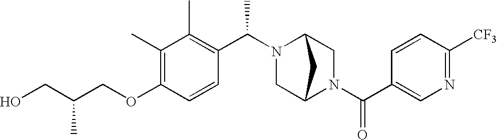 | (2R)-3-{2,3-dimethyl-4-[(1S)-1-((1S,4S)-5-{[6-(tri- fluoromethyl)-pyri- din-3-yl]carbonyl}-2,5-di- azabicyclo[2.2.1]hept-2-yl)ethyl]-phe- noxy}-2-methylpropan-1-ol | 492 |
| 270 | 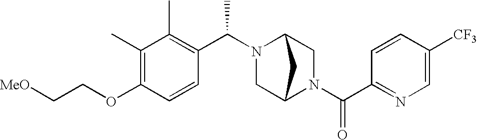 | (1S,4S)-2-{(1S)-1-[4-(2-meth- oxyethoxy)-2,3-dimethyl-phe- nyl]ethyl}-5-{[5-(trifluoro- methyl)pyridin-2-yl]carbonyl}-2,5-di- azabicyclo[2.2.1]heptane | 478 |
| 271 | 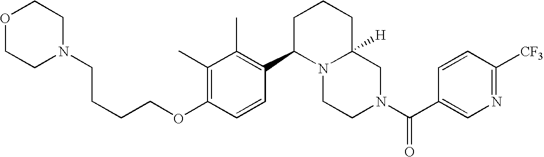 | (6R,9aS)-5-[2,3-dimethyl-4-(4-mor- pholin-4-ylbutoxy)phenyl]-2-{[6-(tri- fluoromethyl)pyridin-3-yl]car- bonyl}octahydro-2H-pyrido[1,2-a]pyra- zine | 575 |
| 272 | 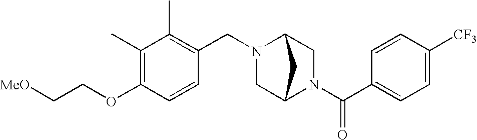 | (1S,4S)-2-[4-(2-methoxyethoxy)-2,3-di- methylbenzyl]-5-[4-(tri- fluoromethyl)benzoyl]-2,5-di- azabicyclo[2.2.1]heptane | 463 |
| 273 | 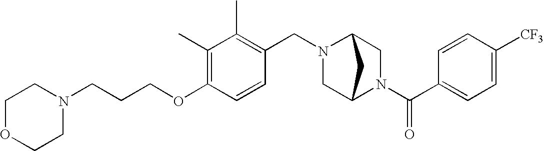 | (1S,4S)-2-[2,3-dimethyl-4-(3-mor- pholin-4-ylpropoxy)benzyl]-5-[4-(tri- fluoromethyl)benzoyl]-2,5-di- azabicyclo[2.2.1]heptane | 532 |
| 274 | 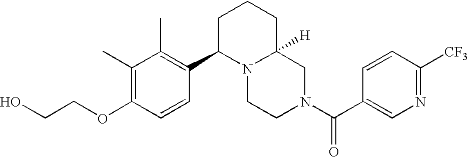 | 2-[2,3-dimethyl-4-((6R,9aS)-2-{[6-(tri- fluoromethyl)pyridin-3-yl]-car- bonyl}octahydro-2H-pyrido[1,2-a]pyra- zin-6-yl)phenoxy]ethanol | 478 |
| 275 | 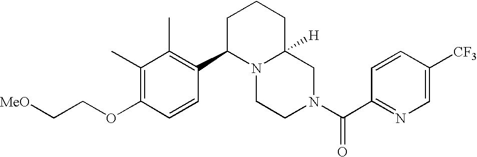 | (6R,9aS)-6-[4-(2-methoxyethoxy)-2,3-di- methylphenyl]-2-{[5-(tri- fluoromethyl)pyridin-2-yl]car- bonyl}octahydro-2H-pyrido[1,2-a]py- razine | 492 |
| 276 | 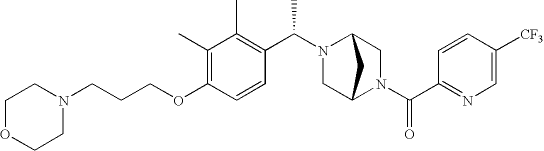 | (1S,4S)-2-{(1S)-1-[2,3-dimethyl-4-(3-mor- pholin-4-ylpropoxy)-phe- nyl]ethyl}-5-{[5-(trifluoro- methyl)pyridin-2-yl]carbonyl}-2,5-di- azabicyclo[2.2.]heptane | 547 |
| 277 | 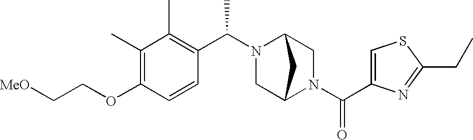 | (1S,4S)-2-[(2-ethyl-1,3-thiazol-4-yl)car- bonyl]-5-{(1S)-1-[4-(2-meth- oxyethoxy)-2,3-dimethyl-phe- nyl]ethyl}-2,5-di- azabicyclo[2.2.1]heptane | 444 |
| 278 | 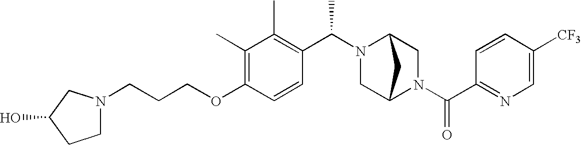 | (3S)-1-(3-{2,3-dimethyl-4-[(1S)-1-((1S,4S)-5-{[5-(tri- fluoromethyl)pyri- din-2-yl]carbonyl}-2,5-di- azabicyclo[2.2.1]hept-2-yl)eth- yl]-phenoxy}pro- pyl)pyrrolidin-3-ol | 547 |
| 279 | 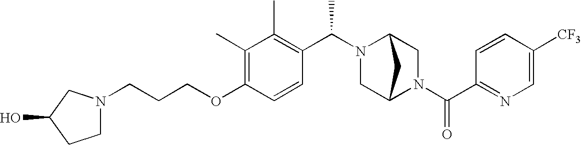 | 1-(3-{2,3-dimethyl-4-[(1S)-1-((1S,4S)-5-{[6-(tri- fluoro-methyl)pyridin-3-yl]car- bonyl}-2,5-di- azabicyclo[2.2.1]hept-2-yl)ethyl]-phe- noxy}propyl)pyrrolidin-3-ol | 547 |
| 280 | 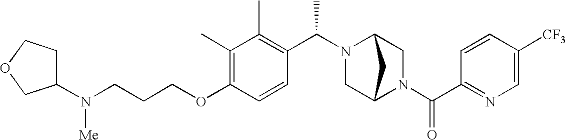 | N-(3-{2,3-dimethyl-4-[(1S)-1-((1S,4S)-5-{[5-(tri- fluoromethyl)-2-pyri- dinyl]carbonyl}-2,5-diaza- bicyclo[2.2.1]hept-2-yl)ethyl]-phe- noxy}propyl)-N-methyltetrahydro-3-fura- namine | 561 |
| 281 | 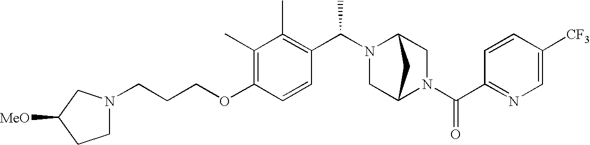 | (1R,4R)-2-[(1R)-1-(4-{3-[(3R)-3-meth- oxy-1-pyrrolidinyl]propoxy}-2,3-di- methylphenyl)ethyl]-5-{[5-(tri- fluoromethyl)-2-pyri- dinyl]carbonyl}-2,5-di- azabicyclo[2.2.1]heptane | 561 |
| 282 | 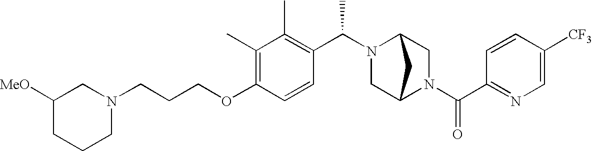 | (1S,4S)-2-((1S)-1-{4-[3-(3-methoxy-1-pipe- ridinyl)propoxy]-2,3-di- methylphenyl}ethyl)-5-{[5-(tri- fluoromethyl)-2-pyri- dinyl]carbonyl}-2,5-di- azabicyclo[2.2.1]heptane | 575 |
| 283 | 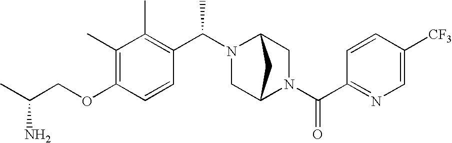 | (2R)-1-{2,3-dimethyl-4-[(1S)-1-((1S,4S)-5-{[6-(tri- fluoromethyl)-3-pyri- dinyl]carbonyl}-2,5-di- azabicyclo[2.2.1]hept-2-yl)eth- yl]phenoxy}-2-propanamine | 477 |
| 284 | 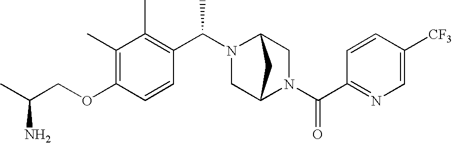 | (2S)-1-{2,3-dimethyl-4-[(1S)-1-((1S,4S)-5-{[6-(tri- fluoromethyl)-3-pyri- dinyl]carbonyl}-2,5-di- azabicyclo[2.2.1]hept-2-yl)eth- yl]phenoxy}-2-propanamine | 477 |
| 285 | 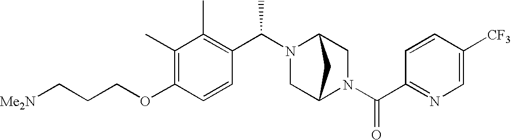 | 3-{2,3-dimethyl-4-[(1S)-1-((1S,4S)-5-{[6-(tri- fluoromethyl)-3-pyri- dinyl]carbonyl}-2,5-di- azabicylco[2.2.1]hept-2-yl)eth- yl]phenoxy}-N,N-dimethyl-1-pro- panamine | 505 |
| 286 | 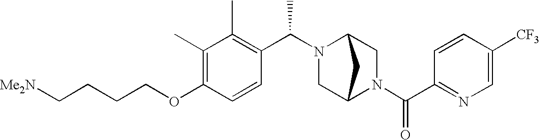 | 4-{2,3-dimethyl-4-[(1S)-1-((1S,4S)-5-{[6-(tri- fluoromethyl)-3-pyri- dinyl]carbonyl}-2,5-di- azabicyclo[2.2.1]hept-2-yl)eth- yl]phenoxy}-N,N-dimethyl-1-butana- mine | 519 |
| 287 | 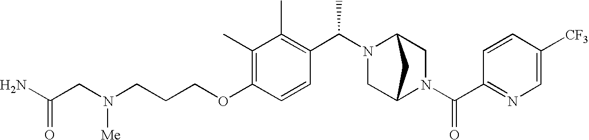 | N˜2˜-(3-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}propyl)-N˜2˜-methyl- glycinamide | 548 |
| 288 | 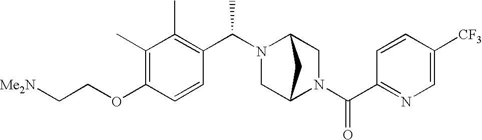 | 2-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}-N,N-di- methylethanamine | 491 |
| 289 | 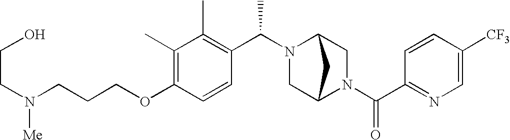 | 2-[(3-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)ethyl]-phe- noxy}propyl)(methyl)amino]-eth- anol | 535 |
| 290 | 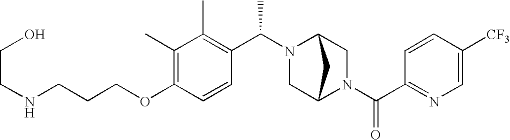 | 2-[(3-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)ethyl]-phe- noxy}propyl)amino]ethanol | 521 |
| 291 | 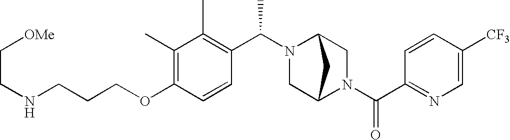 | 3-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}-N-(2-meth- oxyethyl)-1-propanamine | 535 |
| 292 | 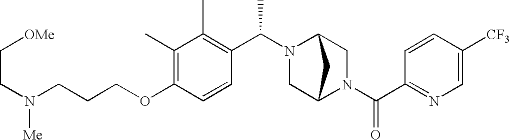 | 3-{2,3-dimethyl-4-[(1S)-1-((1S,4S)-5-{[5-(tri- fluoromethyl)-2-pyridinyl]-car- bonyl}-2,5-diazabicyclo[2.2.1]hept-2-yl)eth- yl]phenoxy}-N-(2-methoxy-eth- yl)-N-methyl-1-propanamine | 549 |
| 293 | 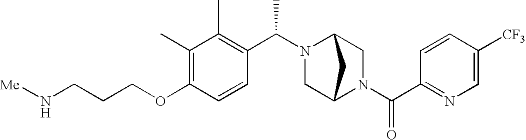 | 3-{2,3-dimethyl-4-[(1S)-1-((1S,4S)-5-{[5-(tri- fluoromethyl)-2-pyridinyl]-car- bonyl}-2,5-diazabicyclo[2.2.1]hept-2-yl)eth- yl]phenoxy}-N-methyl-1-pro- panamine | |
| 294 | 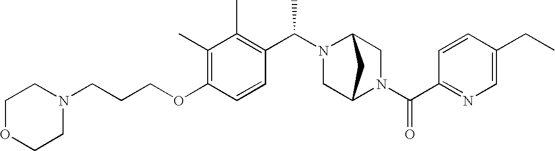 | (1S,4S)-2-((1S)-1-{2,3-dimethyl-4-[3-(4-mor- pholinyl)propoxy]phenyl}-eth- yl)-5-[(5-ethyl-2-pyridinyl)-car- bonyl]-2,5-diazabicyclo[2.2.1]-heptane | 507 |
| 295 | 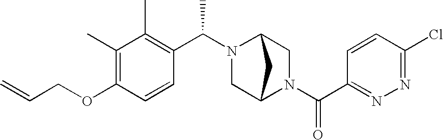 | (1S,4S)-2-{(1S)-1-[4-(allyloxy)-2,3-di- methylphenyl]ethyl}-5-[(6-chloro-3-pyri- dazinyl)carbonyl]-2,5-di- azabicyclo[2.2.1]heptane | 427 |
| 296 | 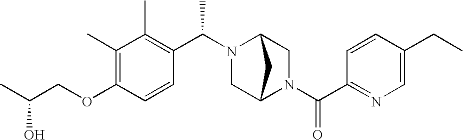 | (2R)-1-[4-((1S)-1-{(1S,4S)-5-[(5-eth- yl-2-pyridinyl)carbonyl]-2,5-di- azabicyclo[2.2.1]hept-2-yl}ethyl)-2,3-di- methylphenoxy]-2-propanol | 438 |
| 297 | 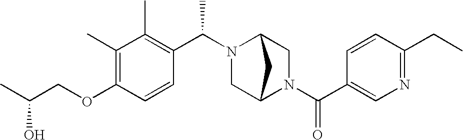 | (2R)-1-[4-((1S)-1-{(1S,4S)-5-[(6-eth- yl-3-pyridinyl)carbonyl]-2,5-di- azabicyclo[2.2.1]hept-2-yl}ethyl)-2,3-di- methylphenoxy]-2-propanol | 438 |
| 298 | 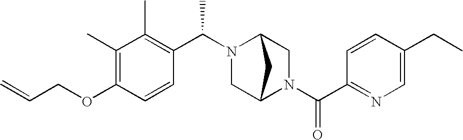 | (1S,4S)-2-{(1S)-1-[4-(allyloxy)-2,3-di- methylphenyl]ethyl}-5-[(5-ethyl-2-pyri- dinyl)carbonyl]-2,5-di- azabicyclo[2.2.1]heptane | 420 |
| 299 | 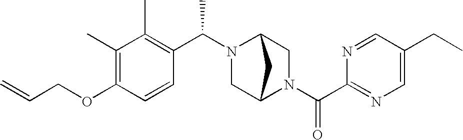 | (1S,4S)-2-{(1S)-1-[4-(allyloxy)-2,3-di- methylphenyl]ethyl}-5-[(5-ethyl-2-pyrimi- dinyl)carbonyl]-2,5-di- azabicyclo[2.2.1]heptane | 421 |
| 300 | 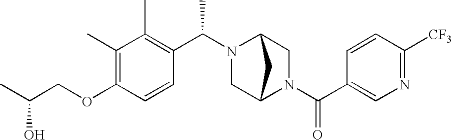 | (2R)-1-{2,3-dimethyl-4-[(1S)-1-((1S,4S)-5-{[6-(tri- fluoromethyl)-3-py- ridinyl]carbonyl}-2,5-di- azabicyclo[2.2.1]hept-2-yl)eth- yl]phenoxy}-2-propanol | 478 |
| 301 | 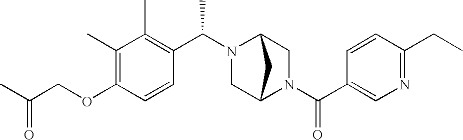 | 1-[4-((1S)-1-{(1S,4S)-5-[(6-ethyl-3-pyri- dinyl)carbonyl]-2,5-di- azabicyclo[2.2.1]hept-2-yl}ethyl)-2,3-di- methylphenoxy]acetone | 436 |
| 302 | 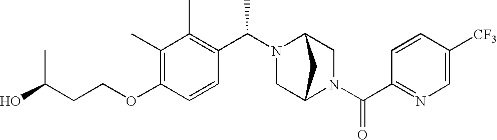 | (2S)-4-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}-2-butanol | 492 |
| 303 | 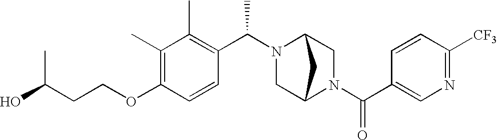 | (2R)-4-{2,3-dimethyl-4-[(1S)-1-((1S,4S)-5-{[6-(tri- fluoromethyl)-3-pyri- dinyl]carbonyl}-2,5-di- azabicyclo[2.2.1]hept-2-yl)eth- yl]phenoxy}-2-butanol | 492 |
| 304 | 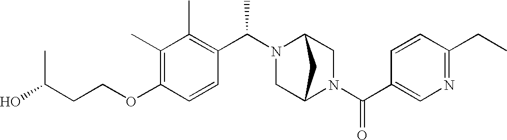 | (2R)-4-[4-((1S)-1-{(1S,4S)-5-[(6-eth- yl-3-pyridinyl)carbonyl]-2,5-di- azabicyclo[2.2.1]hept-2-yl}ethyl)-2,3-di- methylphenoxy]-2-butanol | 452 |
| 305 | 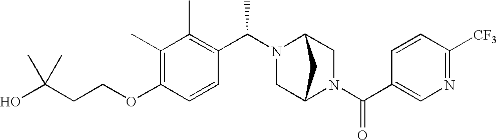 | (2R)-4-[4-((1S)-1-{(1S,4S)-5-[(6-eth- yl-3-pyridinyl)carbonyl]-2,5-di- azabicyclo[2.2.1]hept-2-yl}ethyl)-2,3-di- methylphenoxy]-2-butanol | 506 |
| 306 | 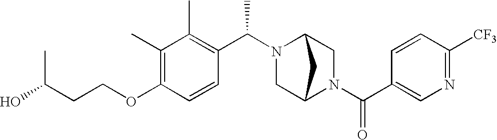 | (2S)-4-{2,3-dimethyl-4-[(1S)-1-((1S,4S)-5-{[6-(tri- fluoromethyl)-3-pyri- dinyl]carbonyl}-2,5-di- azabicyclo[2.2.1]hept-2-yl)eth- yl]phenoxy}-2-butanol | 492 |
| 307 | 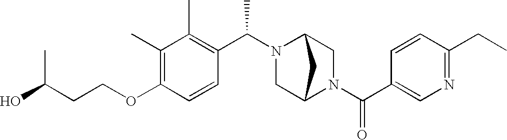 | (2S)-4-[4-((1S)-1-{(1S,4S)-5-[(6-eth- yl-3-pyridinyl)carbonyl]-2,5-di- azabicyclo[2.2.1]hept-2-yl}ethyl)-2,3-di- methylphenoxy]-2-butanol | 452 |
| 308 | 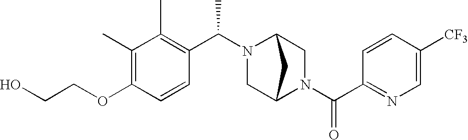 | 2-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}ethanol | 464 |
| 309 | 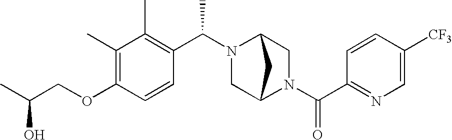 | (2S)-1-{2,3-dimethyl-4-[(1S)-1-((1S,4S)-5-{[5-(tri- fluoromethyl)-2-pyri- dinyl]carbonyl}-2,5-di- aabicyclo[2.2.1]hept-2-yl)eth- yl]phenoxy}-2-propanol | 478 |
| 310 | 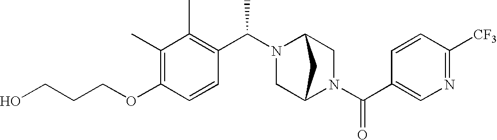 | 3-{2,3-dimethyl-4-[(1S)-1-((1S,4S)-5-{[6-(tri- fluoromethyl)-3-py- ridinyl]carbonyl}-2,5-di- azabicyclo[2.2.1]hept-2-yl)eth- yl]phenoxy}-1-propanol | 478 |
| 311 | 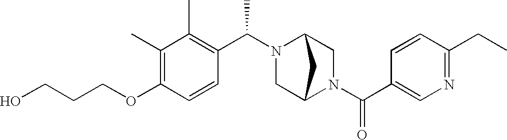 | 3-[4-((1S)-1-{(1S,4S)-5-[(6-ethyl-3-py- ridinyl)carbonyl]-2,5-di- azabicyclo[2.2.1]hept-2-yl}ethyl)-2,3-di- methylphenoxy]-1-propanol | 438 |
| 312 | 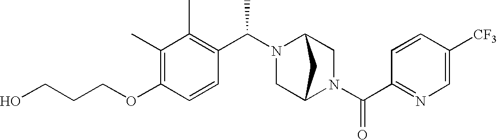 | 3-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}-1-propanol | 478 |
| 313 | 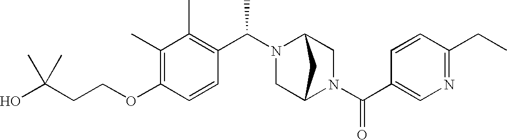 | 4-[4-((1S)-1-{(1S,4S)-5-[(6-ethyl-3-pyri- dinyl)carbonyl]-2,5-di- azabicyclo[2.2.1]hept-2-yl}ethyl)-2,3-di- methylphenoxy]-2-methyl-2-bu- tanol | 466 |
| 314 | 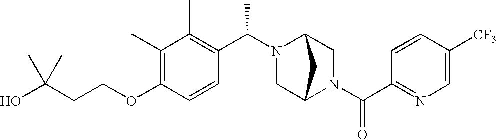 | 4-{2,3-dimethyl-4-[(1S)-1-((1S,4S)-5-{[5-(tri- fluoromethyl)-2-pyri- dinyl]carbonyl}-2,5-di- azabicyclo[2.2.1]hept-2-yl)eth- yl]phenoxy}-2-methyl-2-butanol | 506 |
| 315 | 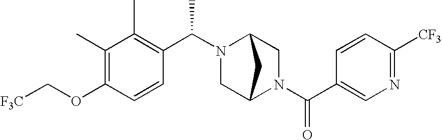 | (1S,4S)-2-{(1S)-1-[2,3-dimethyl-4-(2,2,2-tri- fluoroethoxy)phenyl]ethyl}-5-{[6-(tri- fluoromethyl)-3-pyri- dinyl]carbonyl}-2,5-di- azabicyclo[2.2.1]heptane | 502 |
| 316 | 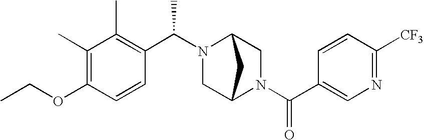 | (1S,4S)-2-[(1S)-1-(4-ethoxy-2,3-di- methylphenyl)ethyl]-5-{[6-(tri- fluoromethyl)-3-pyri- dinyl]carbonyl}-2,5-di- azabicyclo[2.2.1]heptane | 448 |
| 317 | 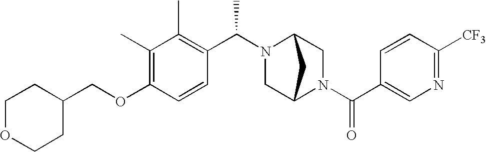 | (1S,4S)-2-{(1S)-1-[2,3-dimethyl-4-(tetra- hydro-2H-pyran-4-yl- methoxy)phenyl]ethyl}-5-{[6-(tri- fluoromethyl)-3-pyri- dinyl]carbonyl}-2,5-di- azabicyclo[2.2.1]heptane | 518 |
| 318 | 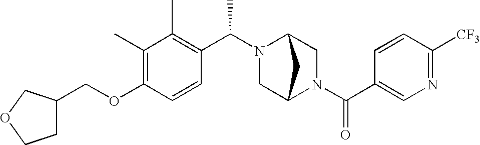 | (1S,4S)-2-{(1S)-1-[2,3-dimethyl-4-(tetra- hydro-2-furanyl- methoxy)phenyl]ethyl}-5-{[6-(tri- fluoromethyl)-3-pyri- dinyl]carbonyl}-2,5-di- azabicyclo[2.2.1]heptane | 504 |
| 319 | 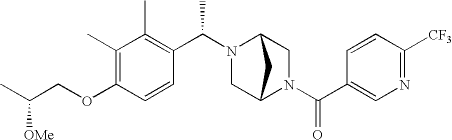 | (1S,4S)-2-[(1S)-1-(4-{[2S)-2-meth- oxypropyl]oxy}-2,3-di- methylphenyl)ethyl]-5-{[6-(tri- fluoromethyl)-3-pyri- dinyl]carbonyl}-2,5-di- azabicyclo[2.2.1]heptane | 492 |
| 320 | 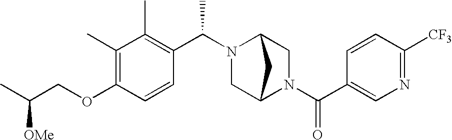 | (1S,4S)-2-[(1S)-1-(4-{[(2)-2-meth- oxypropyl]oxy}-2,3-di- methylphenyl)ethyl]-5-{[6-(tri- fluoromethyl)-3-pyri- dinyl]carbonyl}-2,5-di- azabicyclo[2.2.1]heptane | 492 |
| 321 | 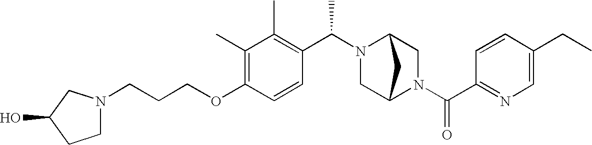 | (3S)-1-{3-[4-((1S)-1-{(1S,4S)-5-[(5-eth- yl-2-pyridinyl)carbonyl]-2,5-di- azabicyclo[2.2.1]hept-2-yl}ethyl)-2,3-di- methylphenoxy]propyl}-3-pyr- rolidinol | 507 |
| 322 | 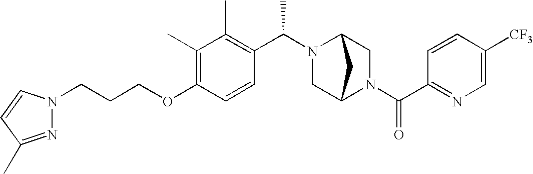 | (1S,4S)-2-((1S)-1-{2,3-dimethyl-4-[3-(3-meth- yl-1H-pyrazol-1-yl)pro- poxy]phenyl}ethyl)-5-{[5-(tri- fluoromethyl)pyridin-2-yl]-car- bonyl}-2,5-di- azabicyclo[2.2.1]heptane | 542 |
| 323 | 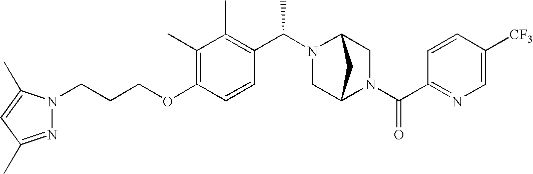 | (1S,4S)-2-((1S)-1-{4-[3-(3,5-dimethyl-1H-py- razol-1-yl)propoxy]-2,3-di- methylphenyl}ethyl)-5-{[5-(tri- fluoromethyl)pyridin-2-yl]car- bonyl}-2,5-di- azabicyclo[2.2.1]heptane | 556 |
| 324 | 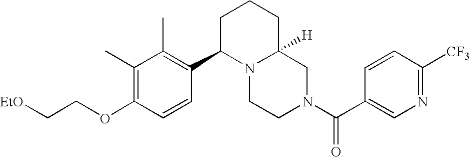 | (6R,9aS)-5-[4-(2-ethoxyethoxy)-2,3-di- methylphenyl]-2-{[6-(trifluoro- methyl)pyridin-3-yl]carbonyl}-octa- hydro-2H-pyrido[1,2-a]pyrazine | 506 |
| 325 | 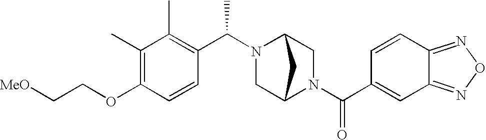 | 5-[((1S,4S)-5-{(1S)-1-[4-(2-meth- oxyethoxy)-2,3-di- methylphenyl]ethyl}-2,5-di- azabicyclo[2.2.1]hept-2-yl)car- bonyl]-2,1,3-benzoxadiazole | 451 |
| 326 | 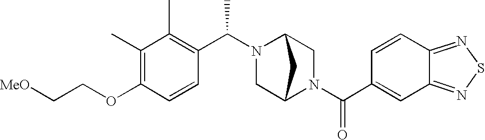 | 5-[((1S,4S)-5-{(1S)-1-[4-(2-meth- oxyethoxy)-2,3-di- methylphenyl]ethyl}-2,5-di- azabicyclo[2.2.1]hept-2-yl)car- bonyl]-2,1,3-benzothiadiazole | 467 |
| 327 | 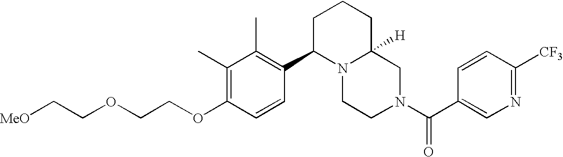 | (6R,9aS)-6-{4-[2-(2-methoxy- ethoxy)ethoxy]-2,3-dimethyl-phenyl}-2-{[6-(tri- fluoromethyl)-pyridin-3-yl]car- bonyl}octahydro-2H-pyrido[1,2-a]py- razine | 536 |
| 328 | 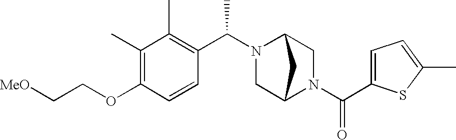 | (1S,4S)-2-{(1S)-1-[4-(2-meth- oxyethoxy)-2,3-di- methylphenyl]ethyl}-5-[(5-methyl-2-thie- nyl)carbonyl]-2,5-di- azabicyclo[2.2.1]heptane | 429 |
| 329 | 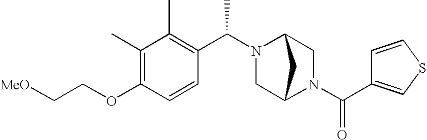 | (1S,4S)-2-{(1S)-1-[4-(2-meth- oxyethoxy)-2,3-di- methylphenyl]ethyl}-5-(3-thienyl- carbonyl)-2,5-di- azabicyclo[2.2.1]heptane | 415 |
| 330 | 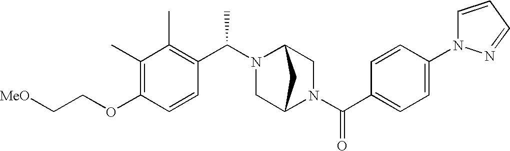 | (1S,4S)-2-{(1S)-1-[4-(2-meth- oxyethoxy)-2,3-di- methylphenyl]ethyl}-5-[4-(1H-pyra- zol-1-yl)benzoyl]-2,5-di- azabicyclo[2.2.1]heptane | 475 |
| 331 | 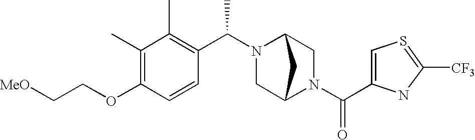 | (1S,4S)-2-{(1S)-1-[4-(2-methoxy- ethoxy)-2,3-dimethylphenyl]ethyl}-5-{[2-(tri- fluoromethyl)-1,3-thiazol-4-yl]car- bonyl}-2,5-di- azabicyclo[2.2.1]heptane | 484 |
| 332 | 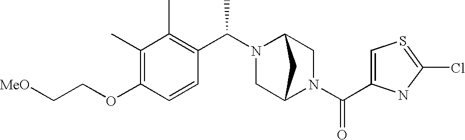 | (1S,4S)-2-[(2-chloro-1,3-thiazol-4-yl)car- bonyl]-5-{(1S)-1-[4-(2-meth- oxyethoxy)-2,3-di- methylphenyl]ethyl}-2,5-di- azabicyclo[2.2.1]heptane | 450 |
| 333 | 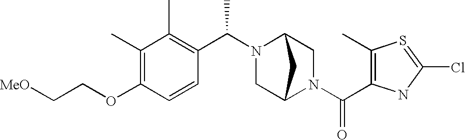 | (1S,4S)-2-[(2-chloro-5-methyl-1,3-thi- azol-4-yl)carbonyl]-5-{(1S)-1-[4-(2-meth- oxyethoxy)-2,3-di- methylphenyl]ethyl}-2,5-di- azabicyclo[2.2.1]heptane | 464 |
| 334 | 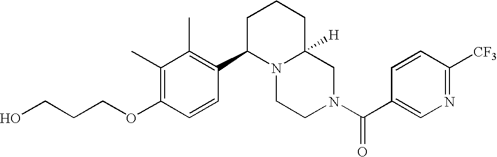 | 3-[2,3-dimethyl-4-((6R,9aS)-2-{[6-(tri- fluoromethyl)pyridin-3-yl]-car- bonyl}octahydro-2H-pyrido[1,2-a]py- razin-6-yl)phenoxy]propan-1-ol | 492 |
| 335 | 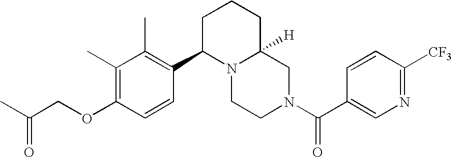 | 1-[2,3-dimethyl-4-((6R,9aS)-2-{[6-(tri- fluoromethyl)pyridin-3-yl]-car- bonyl}octahydro-2H-pyrido[1,2-a]pyra- zin-6-yl)phenoxy]acetone | 490 |
| 336 | 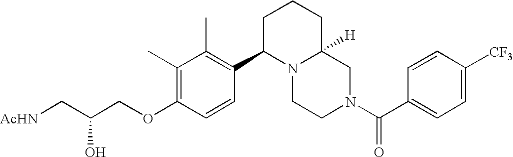 | N-[(2R)-3-(2,3-dimethyl-4-{(6R,9aS)-2-[4-(tri- fluoromethyl)-ben- zoyl]octahydro-2H-pyrido-[1,2-a]py- razin-6-yl}phenoxy)-2-hy- droxypropyl]acetamide | 548 |
| 337 | 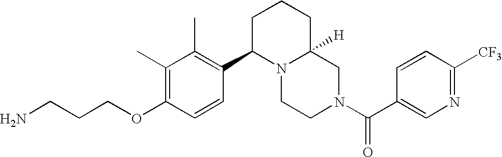 | 3-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]propan-1-amine | 491 |
| 338 | 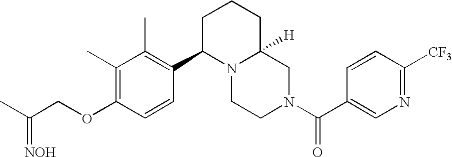 | (2E)-1-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]acetone oxime | 505 |
| 339 | 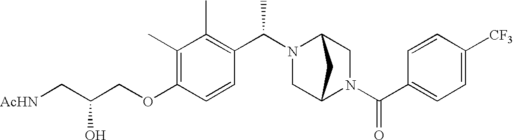 | N-{(2R)-3-[2,3-dimethyl-4-((1S)-1-{(1S,4S)-5-[4-(tri- fluoromethyl)-ben- zoyl]-2,5-diazabicyclo[2.2.1]-hept-2-yl}eth- yl)phenoxy]-2-hydroxy- propyl}acetamide | 534 |
| 340 | 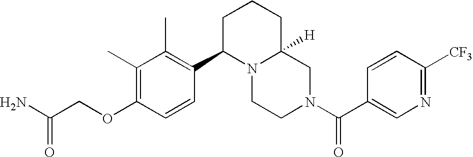 | 2-[2,3-dimethyl-4-((6R,9aS)-2-{[6-(tri- fluoromethyl)pyridin-3-yl]-car- bonyl}octahydro-2H-pyrido[1,2-a]py- razin-6-yl)phenoxy]acetamide | 491 |
| 341 | 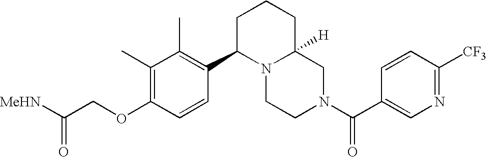 | 2-[2,3-dimethyl-4-((6R,9aS)-2-{[6-(tri- fluoromethyl)pyridin-3-yl]-car- bonyl}octahydro-2H-pyrido[1,2-a]py- razin-6-yl)phenoxy]-N-methyl- acetamide | 505 |
| 342 | 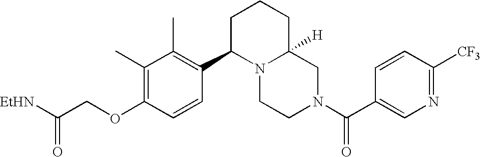 | 2-[2,3-dimethyl-4-((6R,9aS)-2-{[6-(tri- fluoromethyl)pyridin-3-yl]-car- bonyl}octahydro-2H-pyrido[1,2-a]py- razin-6-yl)phenoxy]-N-eth- ylacetamide | 519 |
| 343 | 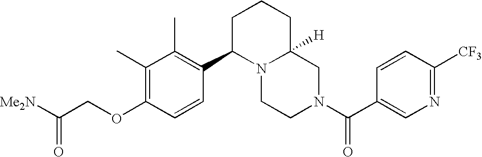 | 2-[2,3-dimethyl-4-((6R,9aS)-2-{[6-(tri- fluoromethyl)pyridin-3-yl]-car- bonyl}octahydro-2H-pyrido[1,2-a]py- razin-6-yl)phenoxy]-N,N-di- methylacetamide | 519 |
| 344 | 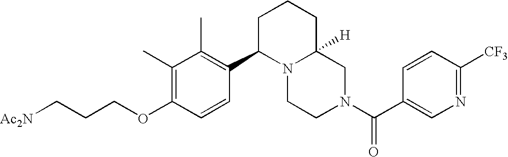 | N-Acetyl-N-(3-{2,3-dimethyl-4-[2-(6-tri- fluoromethyl-pyridin-3-carbonyl)-octa- hydro-pyrido-[1,2-a]py- razin-6-yl]-phenoxy}-pro- pyl)-acetamide | 575 |
| 345 | 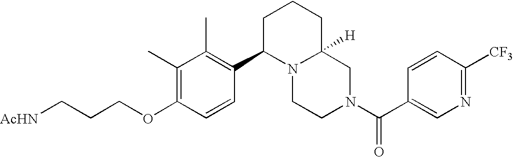 | N-{3-[2,3-dimethyl-4-((6R,9aS)-2-{[6-(tri- fluoromethyl)pyridin-3-yl]-car- bonyl}octahydro-2H-pyrido-[1,2-a]py- razin-6-yl)-phe- noxy]propyl}acetamide | 533 |
| 346 | 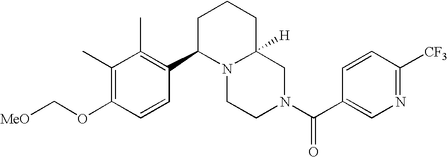 | (6R,9aS)-6-[4-(methoxymethoxy)-2,3-di- methylphenyl]-2-{[6-(tri- fluoromethyl)pyridin-3-yl]-car- bonyl}octahydro-2H-pyrido-[1,2-a]py- razine | 478 |
| 347 | 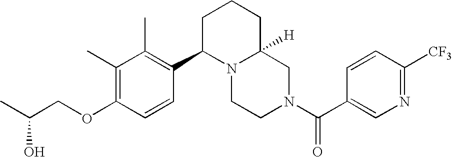 | (2R)-1-[2,3-dimethyl-4-((6R,9aS)-2-{[6-(tri- fluoromethyl)pyridin-3-yl]car- bonyl}octahydro-2H-pyrido[1,2-a]py- razin-6-yl)-phenoxy]propan-2-ol | 492 |
| 348 | 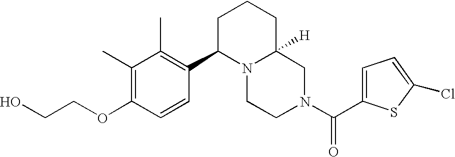 | 2-(4-{6R,9aS)-2-[(5-chloro-2-thi- enyl)carbonyl]octahydro-2H-py- rido[1,2-a]pyrazin-6-yl}-2,3-di- methylphenoxy)ethanol | 449 |
| 349 | 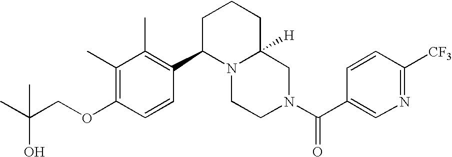 | 1-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]-2-methyl- propan-2-ol | 506 |
| 350 | 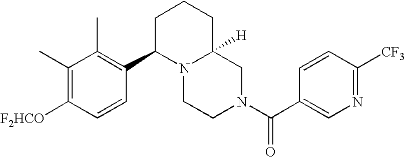 | (6R,9aS)-6-[4-(difluoromethoxy)-2,3-di- methylphenyl]-2-{[6-(tri- fluoromethyl)pyridin-3-yl]-car- bonyl}octahydro-2H-pyrido[1,2-a]py- razine | 484 |
| 351 | 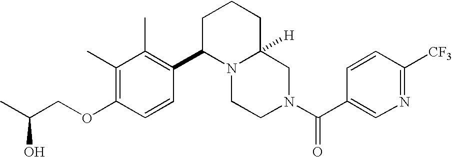 | (2S)-1-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]propan-2-ol | 492 |
| 352 | 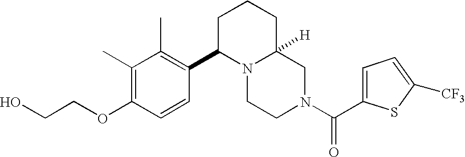 | 2-[2,3-dimethyl-4-((6R,9aS)-2-{[5-(tri- fluoromethyl)-2-thi- enyl]carbonyl}octahydro-2H-pyri- do[1,2-a]pyrazin-6-yl)phe- noxy]ethanol | |
| 353 | 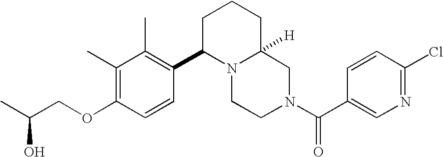 | (2S)-1-[2,3-dimethyl-4-((6R,9aS)-2-{[6-(chloro)pyri- din-3-yl]carbonyl}-octa- hydro-2H-pyrido[1,2-a]pyrazin-6-yl)phe- noxy]propan-2-ol | 458 |
| 354 | 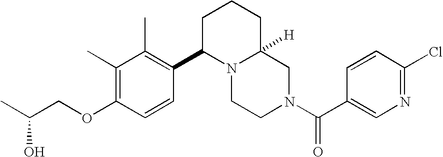 | (2R)-1-[2,3-dimethyl-4-((6R,9aS)-2-{[6-(chloro)py- ridin-3-yl]carbonyl}-octa- hydro-2H-pyrido[1,2-a]pyrazin-6-yl)phe- noxy]propan-2-ol | 458 |
| 355 | 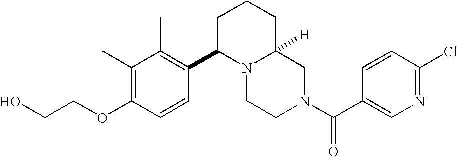 | (6-chloropyridin-3-yl)((6R,9aS)-5-(4-(2-hy- droxyethoxy)-2,3-di- methylphenyl)-hexahydro-1H-pyri- do[1,2-a]pyrazin-2(6H)-yl)meth- anone | 444 |
| 356 | 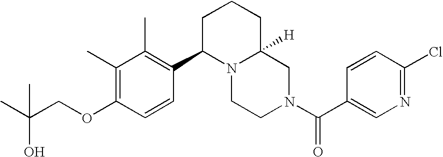 | (6-Chloro-pyridin-3-yl)-{(6R,9aS)-6-[4-(2-hy- droxy-2-methyl-propoxy)-2,3-di- methyl-phenyl]-octahydro-pyri- do[1,2-a]pyrazin-2-yl}-methanone | 472 |
| 357 | 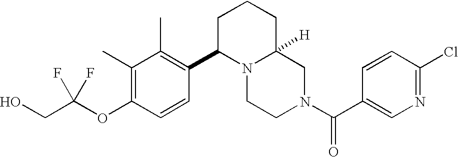 | (6-chloropyridin-3-yl)((6R,9aS)-6-(4-(1,1-di- fluoro-2-hydroxyethoxy)-2,3-di- methylphenyl)-hexahydro-1H-py- rido[1,2-a]pyrazin-2(6H)-yl)meth- anone | 480 |
| 358 | 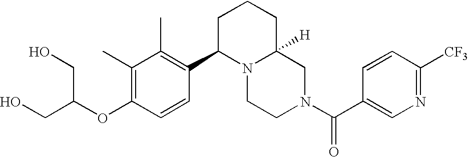 | {(6R,9aS)-6-[4-(2-Hydroxy-1-hy- droxymethyl-ethoxy)-2,3-di- methylphenyl]-octahydro-pyrido-[1,2-a]py- razin-2-yl}-(6-trifluoro- methyl-pyridin-3-yl)-methanone | 508 |
| 359 | 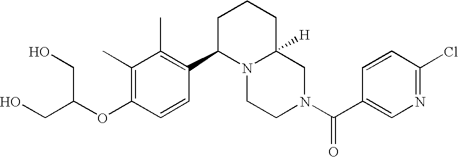 | {(6R,9aS)-6-[4-(2-Hydroxy-1-hy- droxymethyl-ethoxy)-2,3-di- methylphenyl]-octahydro-pyrido-[1,2-a]py- razin-2-yl}-(6-chloro-pyridin-3-yl)-meth- anone | 474 |
| 360 | 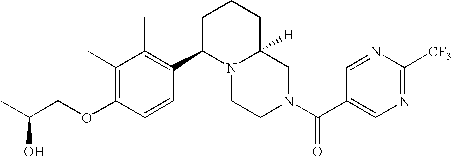 | (2S)-1-[2,3-dimethyl-4-((6R,9aS)-2-{[2-(tri- fluoromethyl)-5-pyri- midinyl]carbonyl}octahydro-2H-pyri- do[1,2-a]pyrazin-6-yl)phenoxy]-2-pro- panol | 493 |
| 361 | 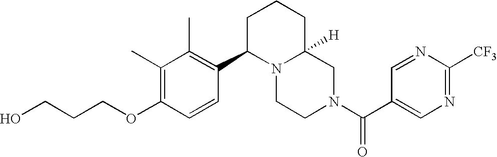 | 3-[2,3-dimethyl-4-((6R,9aS)-2-{[2-(tri- fluoromethyl)-5-pyri- midinyl]carbonyl}octahydro-2H-pyri- do[1,2-a]pyrazin-6-yl)phenoxy]-1-pro- panol | 493 |
| 363 | 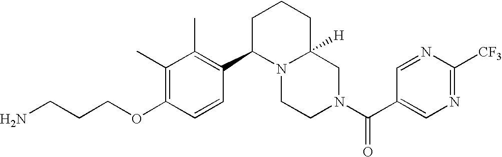 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-1-pro- panamine | 492 |
| 364 | 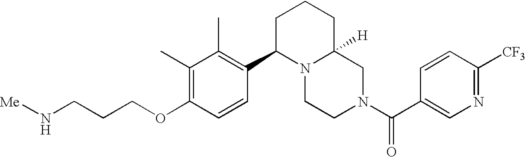 | 3-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]-N-meth- ylpropan-1-amine | 505 |
| 365 | 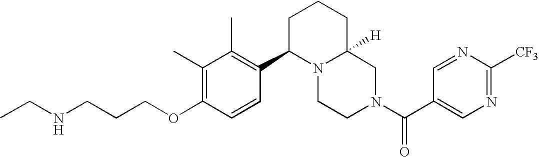 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenyl]-N-ethyl-1-propanamine | 520 |
| 366 | 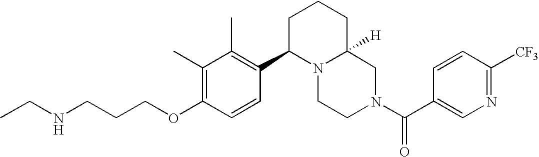 | 3-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]-N-eth- ylpropan-1-amine | 519 |
| 367 | 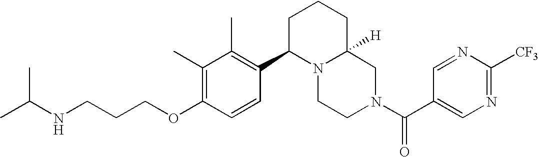 | 3-[2,3-dimethyl-4-((6R,9aS)-2-{[6-(tri- fluoromethyl)-5-pyrimidinyl]-car- bonyl}octahydro-2H-pyrido[1,2-a]py- razin-6-yl)phenoxy]-N-iso- propylpropan-1-amine | 534 |
| 368 | 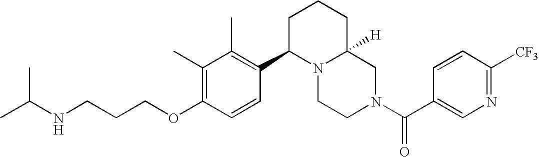 | 3-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]-N-iso- propylpropan-1-amine | 533 |
| 369 | 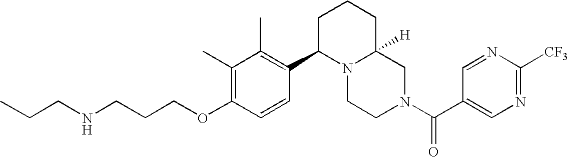 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoy]-N-propyl-1-pro- panamine | 534 |
| 370 | 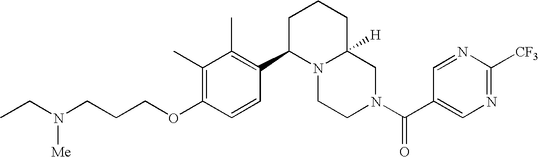 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N-ethyl-N-meth- yl-1-propanamine | 534 |
| 371 | 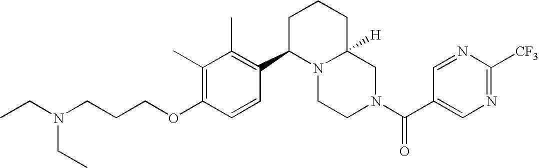 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N,N-diethyl-1-pro- panamine | 547 |
| 372 | 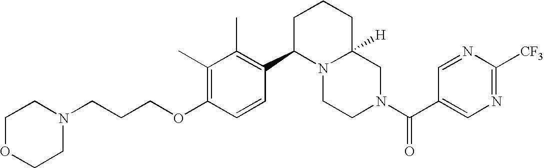 | (6R,9aS)-6-{2,3-dimethyl-4-[3-(4-mor- pholinyl)propoxy]phenyl}-2-{[2-(tri- fluoromethyl)-5-py- rimidinyl]carbonyl}octahydro-2H-py- rido[1,2-a]pyrazine | 562 |
| 373 | 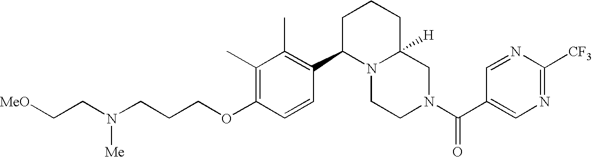 | 3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N-(2-meth- oxyethyl)-N-methyl-1-pro- panamine | 564 |
| 374 | 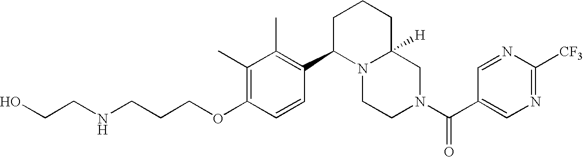 | 2-({3-[2,3-dimethyl-4-((6R,9aS)-2-{[6-(tri- fluoromethyl)pyridin-3-yl]car- bonyl}octahydro-2H-pyrido[1,2-a]py- razin-6-yl)phenoxy]propyl}-a- mino)ethanol | 535 |
| 375 | 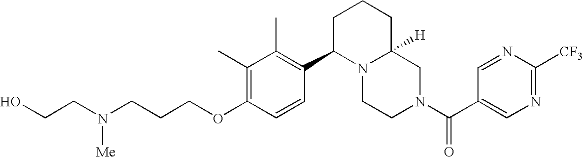 | 2-[{3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]peopyl}-(meth- yl)amino]ethanol | 550 |
| 376 | 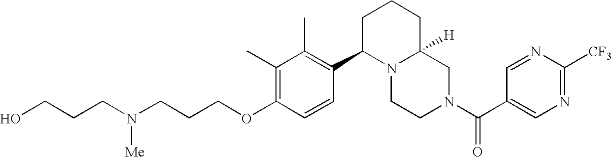 | [(6R,9aS)-6-(4-{3-[(3-Hydroxy-pro- pyl)-methyl-amino]-propoxy}-2,3-di- methylphenyl)-octahydro-py- rido[1,2-a]pyrazin-2-yl]-(2-trifluoro-meth- yl-pyrimidin-5-yl)-methanone | 564 |
| 377 | 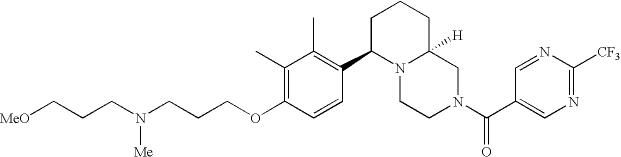 | [(6R,9aS)-6-(4-{3-[(3-Methoxy-pro- pyl)-methyl-amino]-propoxy}-2,3-di- methyl-phenyl)-octahydro-pyrido-[1,2-a]py- razin-2-yl]-(2-trifluoro- methyl-pyrimidin-5-yl)-methanone | 578 |
| 378 | 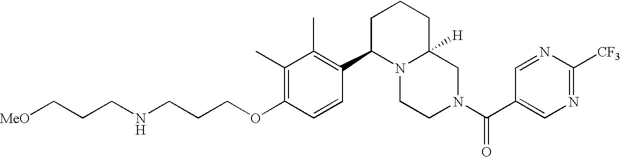 | 3-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phenoxy]-N-(3-meth- oxypropyl)-1-propanamine | 564 |
| 379 | 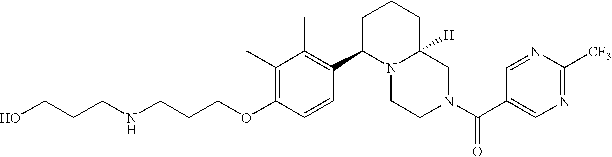 | 3-({3-[2,3-dimethyl-4-((6R,9aS)-2-{[2-(tri- fluoromethyl)-5-py- rimidinyl]carbnonyl}octahydro-2H-py- rido[1,2-a]pyrazin-6-yl)phe- noxy]propyl}amino)-1-propanol | 550 |
| 380 | 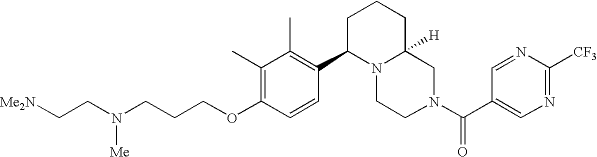 | N-{3-[2,3-dimethyl-4-((6R,9aS)-2-{2-(tri- fluoromethyl)-5-pyrimidinyl]-car- bonyl}octahydro-2H-pyrido[1,2-a]py- razin-6-yl)phenoxy]propyl}-N,N′,N′-tri- methyl-1,2-ethanediamine | 577 |
| 381 | 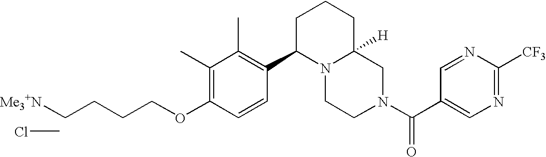 | 4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N,N,N-tri- methyl-1-butanaminium chloride | 549 |
| 382 | 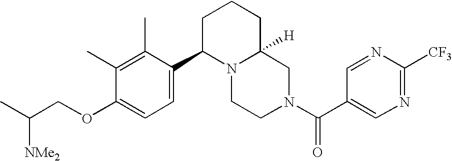 | 1-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N,N-di- methyl-2-propanamine | 520 |
| 383 | 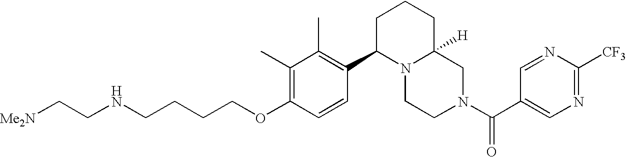 | N′-{4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]butyl}-N,N-di- methyl-1,2-ethanediamine | 577 |
| 384 | 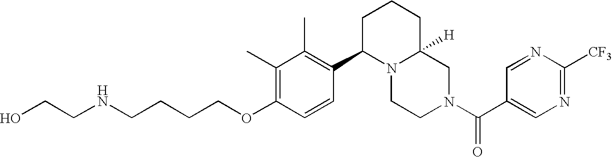 | 2-({4-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phe- noxy]butyl}amino)ethanol | 550 |
| 385 | 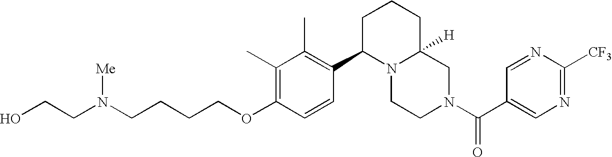 | 2-[{4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]butyl}-(meth- yl)amino]ethanol | 564 |
| 386 | 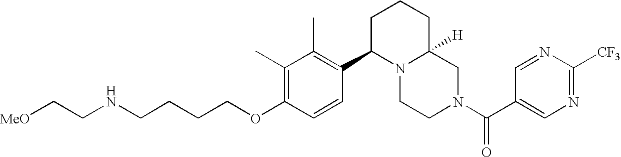 | 4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N-(2-meth- oxyethyl)-1-butanamine | 564 |
| 387 | 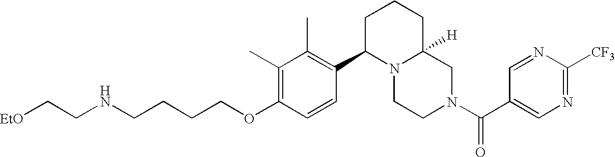 | 4-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phenoxy]-N-(2-eth- oxyethyl)-1-butanamine | 578 |
| 388 | 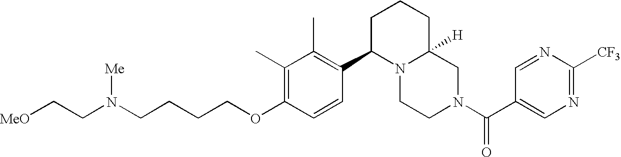 | 4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-N-(2-meth- oxyethyl)-N-methyl-1-butana- mine | 578 |
| 389 | 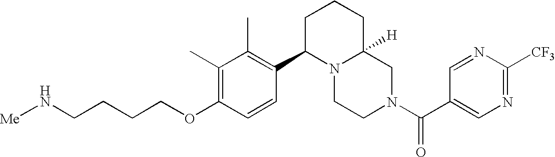 | 4-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phenoxy]-N-meth- yl-1-butanamine | 520 |
| 390 | 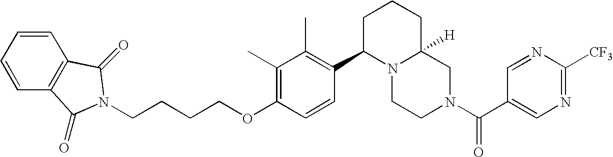 | 2-{3-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]propyl}-1H-iso- indole-1,3(2H)-dione | 622 |
| 391 | 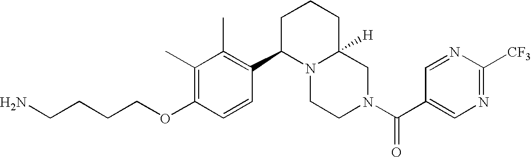 | 4-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phenoxy]-1-butana- mine | 506 |
| 392 | 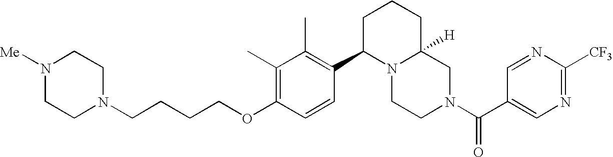 | (6R,9aS)-6-{2,3-dimethyl-4-[4-(4-meth- yl-1-piperazinyl)butoxy]phenyl}-2-{[2-(tri- fluoromethyl)-5-py- rimidinyl]carbonyl}octahydro-2H-py- rido[1,2-a]pyrazine | 589 |
| 393 | 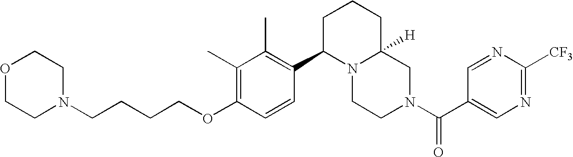 | (6R,9aS)-6-{2,3-dimethyl-4-[4-(4-mor- pholinyl)butoxy]phenyl}-2-{[2-(tri- fluoromethyl)-5-py- rimidinyl]carbonyl}octahydro-2H-py- rido[1,2-a]pyrazine | 576 |
| 394 | 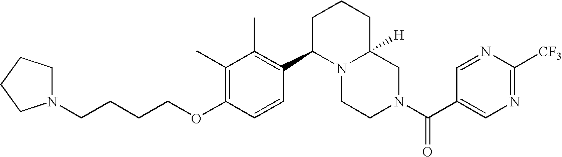 | (6R,9aS)-6-{2,3-dimethyl-4-[4-(1-pyr- rolidinyl)butoxy]phenyl}-2-{[2-(tri- fluoromethyl)-5-py- rimidinyl]carbonyl}octahydro-2H-py- rido[1,2-a]pyrazine | 560 |
| 395 | 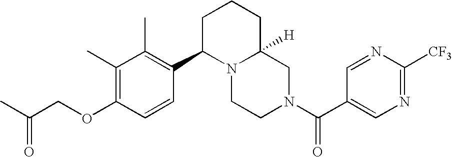 | 1-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phe- noxy]acetone | 491 |
| 396 | 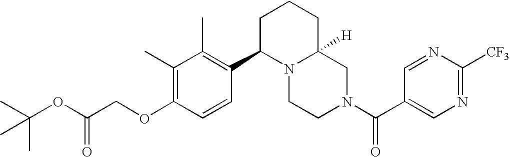 | tert-butyl[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]acetate | 549 |
| 397 | 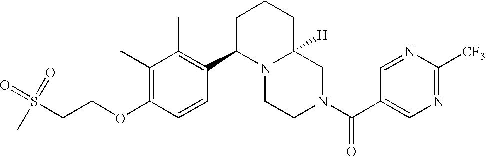 | (6R,9aS)-6-{2,3-dimethyl-4-[2-(meth- ylsulfonyl)ethoxy]phenyl}-2-{[2-(tri- fluoromethyl)-5-py- rimidinyl]carbonyl}octahydro-2H-py- rido[1,2-a]pyrazine | 541 |
| 398 | 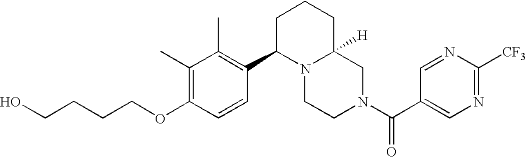 | 4-[2,3-dimethyl-4-((6R,9aS)-2-{[2-(tri- fluoromethyl)-5-py- rimidinyl]carbonyl}octahydro-2H-py- rido[1,2-a]pyrazin-6-yl)phenoxy]-1-bu- tanol | 507 |
| 399 | 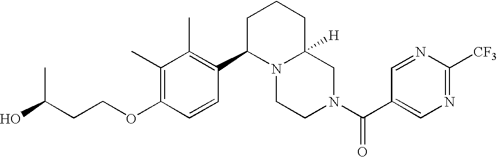 | (2S)-4-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-2-butanol | 507 |
| 400 | 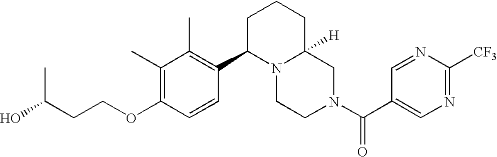 | (2R)-2-[2,3-dimethyl-4-((6R,9aS)-2-{[2-(tri- fluoromethyl)-5-pyrimidinyl]-car- bonyl}octahydro-2H-pyrido[1,2-a]py- razin-6-yl)phenoxy]-2-butanol | 507 |
| 401 | 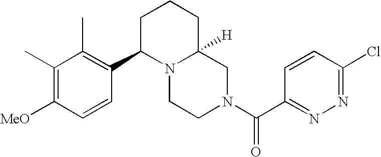 | (6R,9aS)-2-[(6-chloro-3-py- ridazinyl)carbonyl]-6-(4-methoxy-2,3-di- methylphenyl)octahydro-2H-py- rido[1,2-a]pyrazine | 415 |
| 402 | 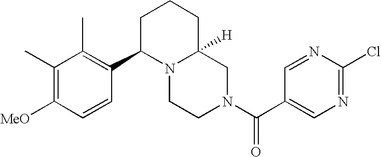 | (6R,9aS)-2-[(2-chloro-5-py- rimidinyl)carbonyl]-6-(4-methoxy-2,3-di- methylphenyl)octahydro-2H-py- rido[1,2-a]pyrazine | 415 |
| 403 | 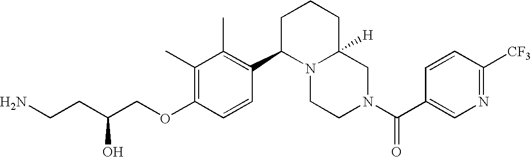 | (2S)-4-amino-1-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-2-bu- tanol | 521 |
| 404 | 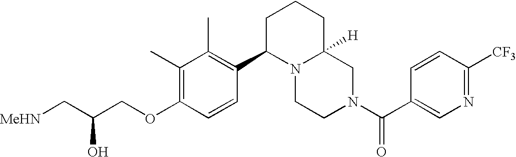 | (2S)-1-[2,3-dimethyl-4-((6R,9aS)-2-{[6-(tri- fluoromethyl)-3-pyridinyl]-car- bonyl}octahydro-2H-pyrido[1,2-a]py- razin-6-yl)phenoxy]-3-(meth- ylamino)-2-propanol | 521 |
| 405 | 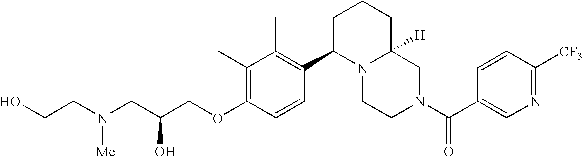 | (2S)-1-[2,3-dimethyl-4-((6R,9aS)-2-{[6-(tri- fluoromethyl)-3-pyridinyl]-car- bonyl}octahydro-2H-pyrido[1,2-a]py- razin-6-yl)phenoxy]-3-[(2-hy- droxyethyl)(methyl)amino]-2-pro- panol | 565 |
| 406 | 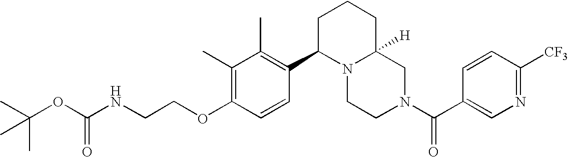 | tert-butyl{2-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-eth- yl}carbamate | 577 |
| 407 | 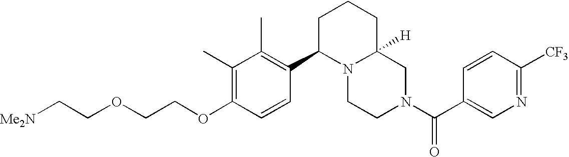 | 2-{2-[2,3-dimethyl-4-((6R,9aS)-2-{[6-(tri- fluoromethyl)-3-pyridinyl]-car- bonyl}octahydro-2H-pyrido[1,2-a]py- razin-6-yl)phenoxy]ethoxy}-N,N-di- methylethanamine | 549 |
| 408 | 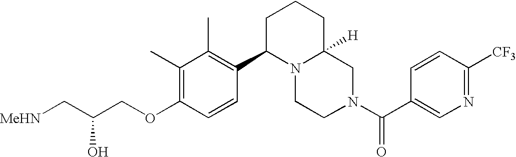 | (2R)-1-[2,3-dimethyl-4-((6R,9aS)-2-{[6-(tri- lfuoromethyl)-3-pyridinyl]-car- bonyl}octahydro-2H-pyrido[1,2-a]py- razin-6-yl)phenoxy]-3-(meth- ylamino)-2-propanol | 521 |
| 409 | 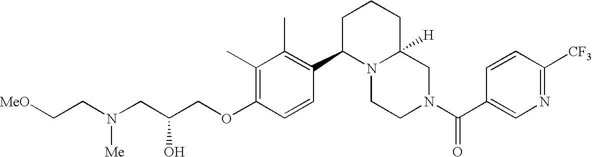 | (2R)-1-[2,3-dimethyl-4-((6R,9aS)-2-{[6-(tri- fluoromethyl)-3-pyridinyl]-car- bonyl}octahydro-2H-pyrido[1,2-a]py- razin-6-yl)phenoxy]-3-[(2-meth- oxyethyl)(methyl)amino]-2-propanol | 579 |
| 410 | 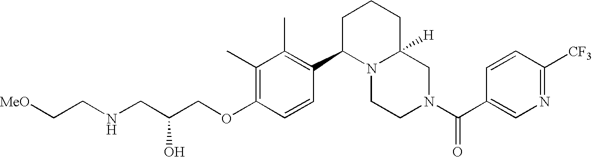 | (2R)-1-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-3-[(2-meth- oxyethyl)amino]-2-propanol | 565 |
| 411 | 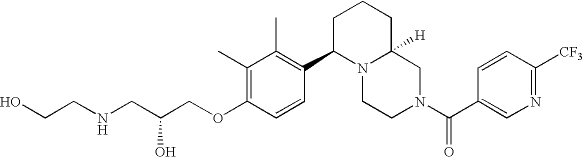 | (2R)-1-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-3-[(2-hy- droxyethyl)amino]-2-propanol | 551 |
| 412 | 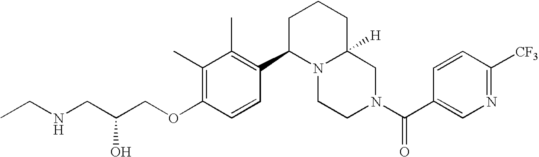 | (2R)-1-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-3-(eth- ylamino)-2-propanol | 535 |
| 413 | 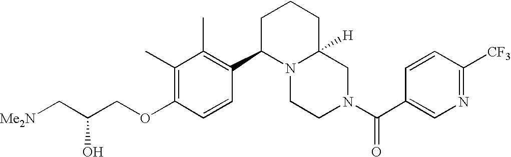 | (2R)-1-(dimethylamino)-3-[2,3-di- methyl-4-((6R,9aS)-2-{[6-(tri- fluoromethyl)-3-py- ridinyl]carbonyl}octahydro-2H-py- rido[1,2-a]pyrazin-6-yl)phenoxy]-2-pro- panol | 535 |
| 414 | 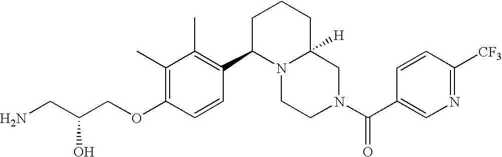 | (2R)-1-amino-3-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-2-pro- panol | 507 |
| 415 | 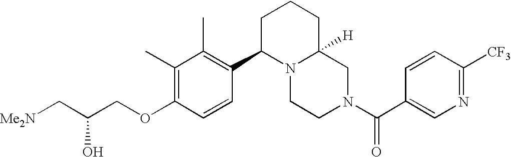 | (6R,9aS)-6-(4-methoxy-2,3-di- methylphenyl)-2-{[1-oxido-6-(tri- fluoromethyl)-3-py- ridinyl]carbonyl}octahydro-2H-py- rido[1,2-a]pyrazine | 534 |
| 416 | 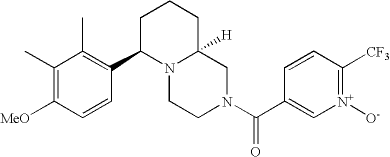 | (6R,9aS)-6-(4-methoxy-2,3-di- methylphenyl)-2-{[1-oxido-6-(tri- fluoromethyl)-3-pyridinyl]-car- bonyl}octahydro-2H-pyrido[1,2-a]py- razine | 463 |
| 417 | 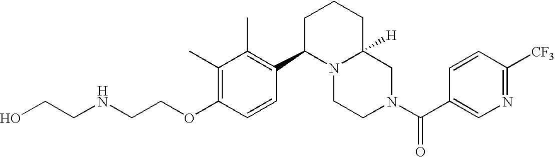 | 2-({2-[2,3-dimethyl-4-((6R,9aS)-2-{[6-(tri- fluoromethyl)-3-py- ridinyl]carbonyl}octahydro-2H-py- rido[1,2-a]pyrazin-6-yl)phe- noxy]ethyl}amino)ethanol | 521 |
| 418 | 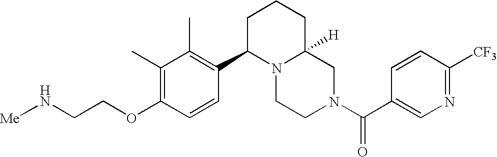 | 2-[2,3-dimethyl-4-((6R,9aS)-2-{[6-(tri- fluoromethyl)-3-py- ridinyl]carbnyl}octahydro-2H-py- rido[1,2-a]pyrazin-6-yl)phenoxy]-N-meth- ylethanamine | 491 |
| 419 | 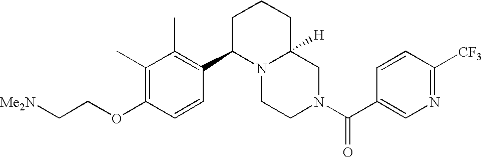 | 2-[2,3-dimethyl-4-((6R,9aS)-2-{[6-(tri- fluoromethyl)-3-py- ridinyl]carbonyl}octahydro-2H-py- rido[1,2-a]pyrazin-6-yl)phenoxy]-N,N-di- methylethanamine | 505 |
| 420 | 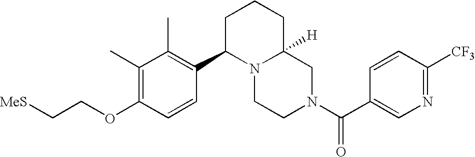 | (6R,9aS)-6-{2,3-dimethyl-4-[2-(meth- ylsulfanyl)ethoxy]phenyl}-2-{[6-(tri- lfuoromethyl)-3-pyridinyl]-car- bonyl}octahydro-2H-pyrido[1,2-a]pyrazine | 508 |

TABLE II

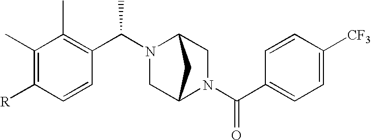

| Cpd. | R (linked via O) | NAME | MS |
|---|---|---|---|
| 421 | 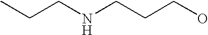 | 3-[2,3-dimethyl-4-(1-{4-[4- (trifluoromethyl)benzoyl]piperazin-1- yl}ethyl)phenoxy]-N-propylpropan-1-amine | 506 |
| 422 | 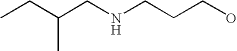 | N-{3-[2,3-dimethyl-4-(1-{4-[4- (trifluoromethyl)benzoyl]piperazin-1- yl}ethyl)phenoxy]propyl}-2-methylbutan-1- amine | 534 |
| 423 | 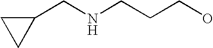 | N-(cyclopropylmethyl)-3-[2,3-dimethyl-4-(1- {4-[4-(trifluoromethyl)benzoyl]piperazin-1- yl}ethyl)phenoxy]propan-1-amine | 518 |
| 424 | 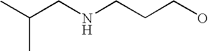 | N-{3-[2,3-dimethyl-4-(1-{4-[4- (trifluoromethyl)benzoyl]piperazin-1- yl}ethyl)phenoxy]propyl}-2-methylpropan-1- amine | 520 |
| 425 | 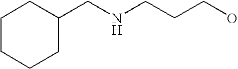 | N-(cyclohexylmethyl)-3-[2,3-dimetbyl-4-(1-{4- [4-(trifluoromethyl)benzoyl]piperazin-1- yl}ethyl)phenoxy]propan-1-amine | 560 |
| 426 | 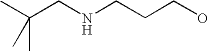 | N-{3-[2,3-dimethyl-4-(1-{4-[4- (trifluoromethyl)benzoyl]piperazin-1- yl}ethyl)phenoxy]propyl}-2,2-dimethylpropan- 1-amine | 534 |
| 427 | 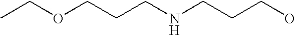 | 3-[2,3-dimethyl-4-(1-{4-[4- (trifluoromethyl)benzoyl]piperazin-1- yl}ethyl)phenoxy]-N-(2-ethoxyethyl)propan-1- amine | 536 |
| 428 | 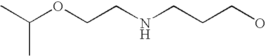 | 3-[2,3-dimethyl-4-(1-{4-[4- (trifluoromethyl)benzoyl]piperazin-1- yl}ethyl)phenoxy]-N-(2- isopropoxyethyl)propan-1-amine | 550 |
| 429 | 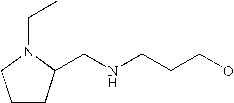 | 3-[2,3-dimethyl-4-(1-{4-[4- (trifluoromethyl)benzoyl]piperazin-1- yl}ethyl)phenoxy]-N-[(1-ethylpyrrolidin-2- yl)methyl]propan-1-amine | 575 |
| 430 | 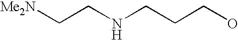 | N′-{3-[2,3-dimethyl-4-(1-{4-[4- (trifluoromethyl)benzoyl]piperazin-1- yl}ethyl)phenoxy]propyl}-N,N- dimethylethane-1,2-diamine | 535 |
| 431 | 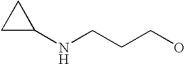 | N-{3-[2,3-dimethyl-4-(1-{4-[4- (trifluoromethyl)benzoyl]piperazin-1- yl}ethyl)phenoxy]propyl}-cyclopropanamine | 504 |
| 432 | 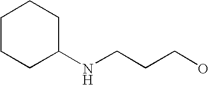 | N-{3-[2,3-dimethyl-4-(1-{4-[4- (trifluoromethyl)benzoyl]piperazin-1- yl}ethyl)phenoxy]propyl}-cyclohexanamine | 546 |
| 433 | 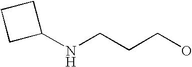 | N-{3-[2,3-dimethyl-4-(1-{4-[4- (trifluoromethyl)benzoyl]piperazin-1- yl}ethyl)phenoxy]propyl}cyclobutanamine | 518 |
| 434 | 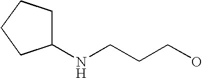 | N-{3-[2,3-dimethyl-4-(1-{4-[4- (trifluoromethyl)benzoyl]piperazin-1- yl}ethyl)phenoxy]propyl}cyclopentanamine | 532 |
| 435 | 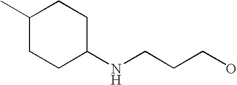 | N-{3-[2,3-dimethyl-4-(1-{4-[4- (trifluoromethyl)benzoyl]piperazin-1- yl}ethyl)phenoxy]propyl}-4- methylcyclohexanamine | 560 |
| 436 | 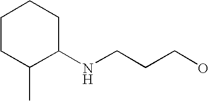 | N-(3-[2,3-dimethyl-4-(1-{4-[4- (trifluoromethyl)benzoyl]piperazin-1- yl}ethyl)phenoxy]propyl)-2- methylcyclohexanamine | 560 |
| 437 | 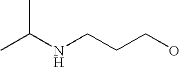 | 3-[2,3-dimethyl-4-(1-{4-[4- (trifluoromethyl)benzoyl]piperazin-1- yl}ethyl)phenoxy]-N-isopropylpropan-1-amine | 506 |
| 438 | 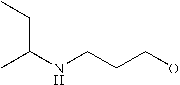 | N-(3-[2,3-dimethyl-4-(1-{4-[4- (trifluoromethyl)benzoyl]piperazin-1- yl}ethyl)phenoxy]propyl}butan-2-amine | 520 |
| 439 | 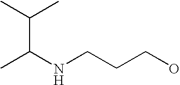 | N-{3-[2,3-dimethyl-4-(1-{4-[4- (trifluoromethyl)benzoyl]piperazin-1- yl}ethyl)phenoxy]propyl}-3-methylbutan-2- amine | 534 |
| 440 | 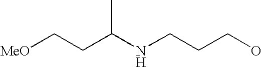 | 3-[2,3-dimethyl-4-(1-{4-[4- (trifluoromethyl)benzoyl]piperazin-1- yl}ethyl)phenoxy]-N-(2-methoxy-1- methylethyl)propan-1-amine | 536 |
| 441 | 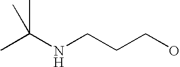 | N-(tert-butyl)-3-[2,3-dimethyl-4-(1-{4-[4- (trifluoromethyl)benzoyl]-piperazin-1- yl}ethyl)phenoxy]-propan-1-amine | 520 |
| 442 | 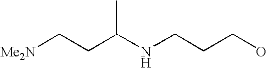 | N²-{3-[2,3-dimethyl-4-(1-{4-[4- (trifluoromethyl)benzoyl]piperazin-1- yl}ethyl)phenoxy]propyl}-N¹,N¹- dimethylpropane-1,2-diamine | 549 |
| 443 | 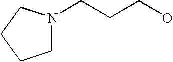 | 1-{1-[2,3-dimethyl-4-(3-pyrrolidin-1- ylpropoxy)phenyl]ethyl}-4-[4- (trifluoromethyl)benzoyl]piperazine | 518 |
| 444 | 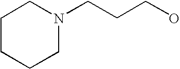 | 1-(1-[2,3-dimethyl-4-(3-piperidin-1- ylpropoxy)phenyl]ethyl}-4-[4- (trifluoromethyl)benzoyl]piperazine | 532 |
| 445 | 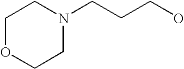 | 4-{3-[2,3-dimethyl-4-(1-{4-[4- (trifluoromethyl)benzoyl]piperazin-1- yl}ethyl)phenoxy]propyl}-morpholine | 534 |
| 446 | 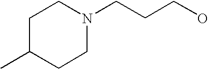 | 1-(1-{2,3-dimethyl-4-[3-(4-methylpiperidin-1- yl)propoxy]phenyl}ethyl)-4-[4- (trifluoromethyl)benzoyl]piperazine | 546 |
| 447 | 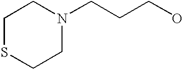 | 4-{3-[2,3-dimethyl-4-(1-{4-[4- (trifluoromethyl)benzoyl]piperazin-1- yl}ethyl)phenoxy]propyl}-thiomorpholine | 550 |
| 448 | 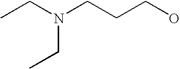 | 3-[2,3-dimethyl-4-(1-{4-[4- (trifluoromethyl)benzoyl]piperazin-1- yl}ethyl)phenoxy]-N,N-diethylpropan-1-amine | 520 |
| 449 | 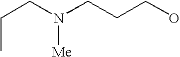 | 3-[2,3-dimethyl-4-(1-{4-[4- (trifluoromethyl)benzoyl]piperazin-1- yl}ethyl)phenoxy]-N-methyl-N-propylpropan- 1-amine | 520 |
| 450 | 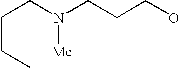 | N-{3-[2,3-dimethyl-4-(1-{4-[4- (trifluoromethyl)benzoyl]piperazin-1- yl}ethyl)phenoxy]propyl}-N-methylbutan-1- amine | 534 |
| 451 | 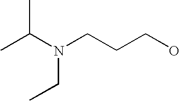 | 3-[2,3-dimethyl-4-(1-{4-[4- (trifluoromethyl)benzoyl]piperazin-1- yl}ethyl)phenoxy]-N-ethyl-N-isopropylpropan- 1-amine | 534 |
| 452 | 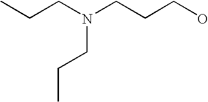 | 3-[2,3-dimethyl-4-(1-{4-[4- (trifluoromethyl)benzoyl]piperazin-1- yl}ethyl)phenoxy]-N,N-dipropylpropan-1- amine | 548 |
| 453 | 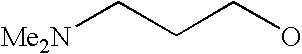 | 3-[2,3-dimethyl-4-(1-{4-[4- (trifluoromethyl)benzoyl]piperazin-1- yl}ethyl)phenoxy]-N,N-dimethylpropan-1- amine | 492 |
| 454 | 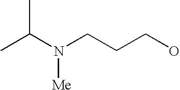 | 3-[2,3-dimethyl-4-(1-{4-[4- (trifluoromethyl)benzoyl]piperazin-1- yl}ethyl)phenoxy]-N-isopropyl-N- methylpropan-1-amine | 520 |
| 455 | 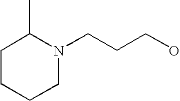 | 1-(1-{2,3-dimethyl-4-[3-(2-methylpiperidin-1- yl)propoxy]phenyl}ethyl)-4-[4- (trifluoromethyl)benzoyl]piperazine | 546 |
| 456 | 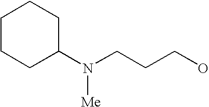 | N-{3-[2,3-dimethyl-4-(1-{4-[4- (trifluoromethyl)benzoyl]piperazin-1- yl}ethyl)phenoxy]propyl}-N- methylcyclohexanamine | 560 |
| 457 | 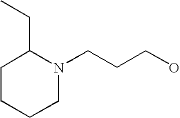 | 1-(1-{4-[3-(2-ethylpiperidin-1-yl)propoxy]-2,3- dimethylphenyl}ethyl)-4-[4- (trifluoromethyl)benzoyl]piperazine | 560 |
| 458 | 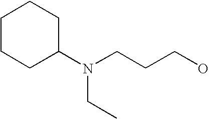 | N-{3-[2,3-dimethyl-4-(1-{4-[4- (trifluoromethyl)benzoyl]piperazin-1- yl}ethyl)phenoxy]propyl}-N- ethylcyclohexanamine | 574 |
| 459 | 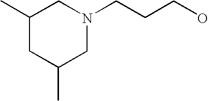 | 1-(1-{4-[3-(3,5-dimethylpiperidin-1- yl)propoxy]-2,3-dimethylphenyl}ethyl)-4-[4- (trifluoromethyl)benzoyl]piperazine | 560 |
| 460 | 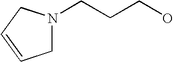 | 1-(1-{4-[3-(2,5-dihydro-1H-pyrrol-1- yl)propoxy]-2,3-dimethylphenyl}ethyl)-4-[4- (trifluoromethyl)benzoyl]piperazine | 516 |
| 461 | 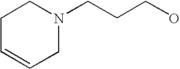 | 1-(1-{4-[3-(3,6-dihydropyridin-1(2H)- yl)propoxy]-2,3-dimethylphenyl}ethyl)-4-[4- (trifluoromethyl)benzoyl]piperazine | 530 |
| 462 | 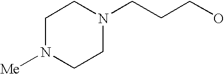 | 1-(1-{2,3-dimethyl-4-[3-(4-methylpiperazin-1- yl)propoxy]phenyl}ethyl)-4-[4- (trifluoromethyl)benzoyl]piperazine | 547 |
| 463 | 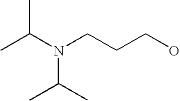 | 3-[2,3-dimethyl-4-(1-{4-[4- (trifluoromethyl)benzoyl]piperazin-1- yl}ethyl)phenoxy]-N,N-diisopropylpropan-1- amine | 548 |
| 464 | 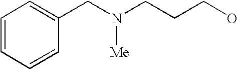 | N-benzyl-3-[2,3-dimethyl-4-(1-{4-[4- (trifluoromethyl)benzoyl]-piperazin-1- yl}ethyl)phenoxy]-N-methylpropan-1-amine | 568 |
| 465 | 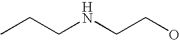 | N-{2-[2,3-dimethyl-4-(1-{4-[4- (trifluoromethyl)benzoyl]piperazin-1- yl)ethyl)phenoxy]ethyl}propan-1-amine | 492 |
| 466 | 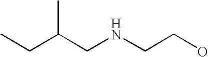 | N-{2-[2,3-dimethyl-4-(1-{4-[4- (trifluoromethyl)benzoyl]piperazin-1- yl}ethyl)phenoxy]ethyl}-2-methylbutan-1- amine | 520 |
| 467 | 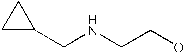 | N-(cyclopropylmethyl)-2-[2,3-dimethyl-4-(1- N{4-[4-(trifluoromethyl)benzoyl]piperazin-1- yl}ethyl)phenoxy]ethanamine | 504 |
| 468 | 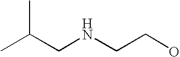 | N-{2-[2,3-dimethyl-4-(1-{4-[4- (trifluoromethyl)benzoyl]piperazin-1- yl}ethyl)phenoxylethyl}-2-methylpropan-1- amine | 506 |
| 469 | 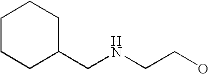 | N-(cyclohexylmethyl)-2-[2,3-dimethyl-4-(1-{4- [4-(trifluoromethyl)benzoyl]piperazin-1- yl}ethyl)phenoxy]ethanamine | 546 |
| 470 | 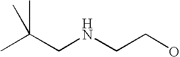 | N-{2-[2,3-dimethyl-4-(1-{4-[4- (trifluoromethyl)benzoyl]piperazin-1- yl}ethyl)phenoxy]ethyl}-2,2-dimethylpropan-1- amine | 520 |
| 471 | 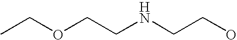 | 2-[2,3-dimethyl-4-(1-{4-[4- (trifluoromethyl)benzoyl]piperazin-1- yl}ethyl)phenoxy]-N-(2- ethoxyethyl)ethanamine | 522 |
| 472 | 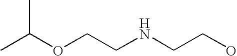 | 2-[2,3-dimethyl-4-(1-{4-[4- (trifluoromethyl)benzoyl]piperazin-1- yl}ethyl)phenoxy]-N-(2- isopropoxyethyl)ethanamine | 536 |
| 473 | 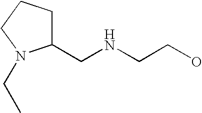 | 2-[2,3-dimethyl-4-(1-{4-[4- (trifluoromethyl)benzoyl]piperazin-1- yl}ethyl)phenoxy]-N-[(1-ethylpyrrolidin-2- yl)methyl]ethanamine | 561 |
| 474 | 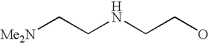 | N′-{2-[2,3-dimethyl-4-(1-{4-[4- (trifluoromethyl)benzoyl]piperazin-1- yl}ethyl)phenoxy]ethyl}-N,N-dimethylethane- 1,2-diamine | 521 |
| 475 | 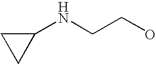 | N-{2-[2,3-dimethyl-4-(1-{4-[4- (trifluoromethyl)benzoyl]piperazin-1- yl}ethyl)phenoxy]ethyl}-cyclopropanamine | 490 |
| 476 | 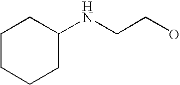 | N-{2-[2,3-dimethyl-4-(1-{4-[4- (trifluoromethyl)benzoyl]piperazin-1- yl}ethyl)phenoxy]ethyl}cyclohexanamine | 532 |
| 477 | 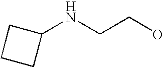 | N-{2-[2,3-dimethyl-4-(1-{4-[4- (trifluoromethyl)benzoyl]piperazin-1- yl}ethyl)phenoxy]ethyl}cyclobutanamine | 504 |
| 478 | 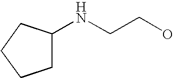 | N-{2-[2,3-dimethyl-4-(1-{4-[4- (trifluoromethyl)benzoyl]piperazin-1- yl}ethyl)phenoxy]ethyl}cyclopentanamine | 518 |
| 479 | 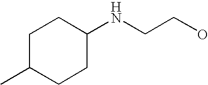 | N-{2-[2,3-dimethyl-4-(1-{4-[4- (trifluoromethyl)benzoyl]piperazin-1- yl}ethyl)phenoxy]ethyl}-4- methylcyclohexanamine | 546 |
| 480 | 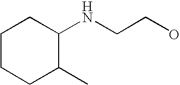 | N-{2-[2,3-dimethyl-4-(1-{4-[4- (trifluoromethyl)benzoyl]piperazin-1- yl}ethyl)phenoxy]ethyl}-2- methylcyclohexanamine | 546 |
| 481 | 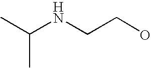 | N-{2-[2,3-dimethyl-4-(1-{4-[4- (trifluoromethyl)benzoyl]piperazin-1- yl}ethyl)phenoxy]ethyl}propan-2-amine | 492 |
| 482 | 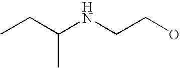 | N-{2-[2,3-dimethyl-4-(1-{4-[4- (trifluoromethyl)benzoyl]piperazin-1- yl}ethyl)phenoxy]ethyl}butan-2-amine | 506 |
| 483 | 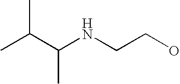 | N-{2-[2,3-dimethyl-4-(1-{4-[4- (trifluoromethyl)benzoyl]piperazin-1- yl}ethyl)phenoxy]ethyl}-3-methylbutan-2- amine | 520 |
| 484 | 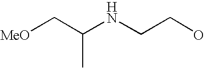 | N-{2-[2,3-dimethyl-4-(1-{4-[4- (trifluoromethyl)benzoyl]piperazin-1- yl}ethyl)phenoxy]ethyl}-1-methoxypropan-2- amine | 522 |
| 485 | 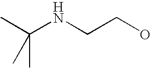 | N-{2-[2,3-dimethyl-4-(1-{4-[4- (trifluoromethyl)benzoyl]piperazin-1- yl}ethyl)phenoxy]ethyl}-2-methylpropan-2- amine | 506 |
| 486 | 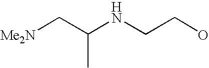 | N²-{2-[2,3-dimethyl-4-(1-{4-[4- (trifluoromethyl)benzoyl]piperazin-1- yl}ethyl)phenoxy]ethyl}-N¹,N¹- dimethylpropane-1,2-diamine | 535 |
| 487 | 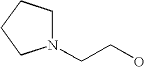 | 1-{1-[2,3-dimethyl-4-(2-pyrrolidin-1- ylethoxy)phenyl]ethyl}-4-[4- (trifluoromethyl)benzoyl]piperazine | 504 |
| 488 | 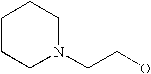 | 1-{1-[2,3-dimethyl-4-(2-piperidin-1- ylethoxy)phenyl]ethyl}-4-[4- (trifluoromethyl)benzoyl]piperazine | 518 |
| 489 | 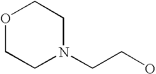 | 4-{2-[2,3-dimethyl-4-(1-{4-[4- (trifluoromethyl)benzoyl]piperazin-1- yl}ethyl)phenoxy]ethyl}morpholine | 520 |
| 490 | 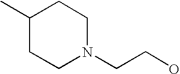 | 1-(1-{2,3-dimethyl-4-[2-(4-methylpiperidin-1- yl)ethoxy]phenyl}ethyl)-4-[4- (trifluoromethyl)benzoyl]piperazine | 532 |
| 491 | 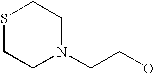 | 4-{2-[2,3-dimethyl-4-(1-{4-[4- (trifluoromethyl)benzoyl]piperazin-1- yl}ethyl)phenoxy]ethyl}thiomorpholine | 536 |
| 492 | 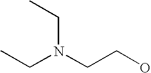 | 2-[2,3-dimethyl-4-(1-{4-[4- (trifluoromethyl)benzoyl]piperazin-1- yl}ethyl)phenoxy]-N,N-diethylethanamine | 506 |
| 493 | 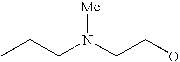 | N-{2-[2,3-dimethyl-4-(1-{4-[4- (trifluoromethyl)benzoyl]piperazin-1- yl}ethyl)phenoxy]ethyl}-N-methylpropan-1- amine | 506 |
| 494 | 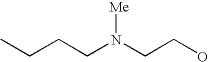 | N-{2-[2,3-dimethyl-4-(1-{4-[4- (trifluoromethyl)benzoyl]piperazin-1- yl}ethyl)phenoxy]ethyl}-N-methylbutan-1- amine | 520 |
| 495 | 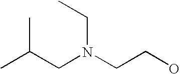 | N-{2-[2,3-dimethyl-4-(1-{4-[4- (trifluoromethyl)benzoyl]piperazin-1- yl}ethyl)phenoxy]ethyl}-N-ethylpropan-2- amine | 520 |
| 496 | 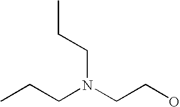 | N-{2-[2,3-dimethyl-4-(1-{4-[4- (trifluoromethyl)benzoyl]piperazin-1- yl}ethyl)pbenoxy]ethyl}-N-propylpropan-1- amine | 534 |
| 497 | 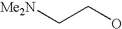 | 2-[2,3-dimethyl-4-(1-{4-[4- (trifluoromethyl)benzoyl]piperazin-1- yl}ethyl)phenoxy]-N,N-dimethylethanamine | 478 |
| 498 | 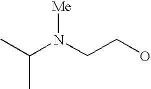 | N-{2-[2,3-dimethyl-4-(1-{4-[4- (trifluoromethyl)benzoyl]piperazin-1- yl}ethyl)phenoxy]ethyl}-N-methylpropan-2- amine | 506 |
| 499 | 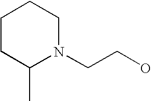 | 1-(1-{2,3-dimethyl-4-[2-(2-methylpiperidin-1- yl)ethoxy]phenyl}ethyl)-4-[4- (trifluoromethyl)benzoyl]piperazine | 532 |
| 500 | 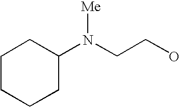 | N-{2-[2,3-dimethyl-4-(1-{4-[4- (trifluoromethyl)benzoyl]piperazin-1- yl}ethyl)phenoxy]ethyl}-N- methylcyclohexanamine | 546 |
| 501 | 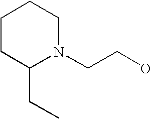 | 1-(1-{4-[2-(2-ethylpiperidin-1-yl)ethoxy]-2,3- dimethylphenyl}ethyl)-4-[4- (trifluoromethyl)benzoyl]piperazine | 546 |
| 502 | 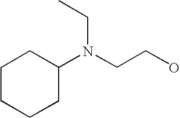 | N-{2-[2,3-dimethyl-4-(1-{4-[4- (trifluoromethyl)benzoyl]piperazin-1- yl}ethyl)phenoxy]ethyl}-N- ethylcyclohexanamine | 560 |
| 503 | 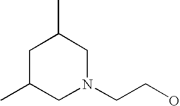 | 1-(1-{4-[2-(3,5-dimethylpiperidin-1-yl)ethoxy]- 2,3-dimethylphenyl}ethyl)-4-[4- (trifluoromethyl)benzoyl]piperazine | 546 |
| 504 | 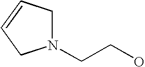 | 1-(1-{4-[2-(2,5-dihydro-1H-pyrrol-1- yl)ethoxy]-2,3-dimethylphenyl}ethyl)-4-[4- (trifluoromethyl)benzoyl]piperazine | 502 |
| 505 | 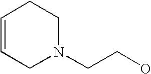 | 1-(1-{4-[2-(3,6-dihydropyridin-1(2H)- yl)ethoxy]-2,3-dimethylphenyl}ethyl)-4-[4- (trifluoromethyl)benzoyl]piperazine | 516 |
| 506 | 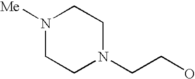 | 1-(1-{2,3-dimethyl-4-[2-(4-methylpiperazin-1- yl)ethoxy]phenyl}ethyl)-4-[4- (trifluoromethyl)benzoyl]piperazine | 533 |
| 507 | 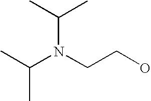 | N-{2-[2,3-dimethyl-4-(1-{4-[4- (trifluoromethyl)benzoyl]piperazin-1- yl}ethyl)phenoxy]ethyl}-N-isopropylpropan-2- amine | 534 |
| 508 | 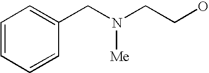 | N-benzyl-2-[2,3-dimethyl-4-(1-{4-[4- (trifluoromethyl)benzoyl]piperazin-1- yl}ethyl)phenoxy]-N-methylethanamine | 554 |
| 509 | 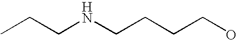 | 4-[2,3-dimethyl-4-(1-{4-[4- (trifluoromethyl)benzoyl]piperazin-1- yl}ethyl)phenoxy]-N-propylbutan-1-amine | 520 |
| 510 | 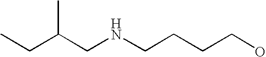 | N-{4-[2,3-dimethyl-4-(1-{4-[4- (trifluoromethyl)benzoyl]piperazin-1- yl}ethyl)phenoxy]butyl}-2-methylbutan-1- amine | 548 |
| 511 | 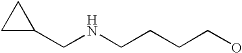 | N-(cyclopropylmethyl)-4-[2,3-dimethyl-4-(1- {4-[4-(trifluoromethyl)benzoyl]piperazin-1- yl}ethyl)phenoxy]butan-1-amine | 532 |
| 512 | 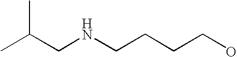 | 4-[2,3-dimethyl-4-(1-{4-[4- (trifluoromethyl)benzoyl]piperazin-1- yl}ethyl)phenoxy]-N-isobutylbutan-1-amine | 534 |
| 513 | 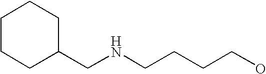 | N-(cyclohexylmethyl)-4-[2,3-dimethyl-4-(1-{4- [4-(trifluoromethyl)benzoyl]piperazin-1- yl}ethyl)phenoxy]butan-1-amine | 574 |
| 514 | 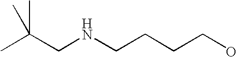 | N-(2,2-dimethylpropyl)-4-[2,3-dimethyl-4-(1- {4-[4-(trifluoromethyl)benzoyl]piperazin-1- yl}ethyl)phenoxy]butan-1-amine | 548 |
| 515 | 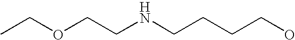 | 4-[2,3-dimethyl-4-(1-{4-[4- (trifluoromethyl)benzoyl]piperazin-1- yl}ethyl)phenoxy]-N-(2-ethoxyethyl)butan-1- amine | 550 |
| 516 | 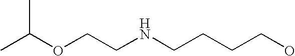 | 4-[2,3-dimethyl-4-(1-{4-[4- (trifluoromethyl)benzoyl]piperazin-1- yl}ethyl)phenoxy]-N-(2-isopropoxyethyl)butan- 1-amine | 564 |
| 517 | 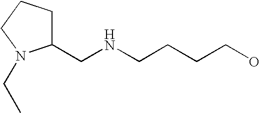 | 4-[2,3-dimethyl-4-(1-{4-[4- (trifluoromethyl)benzoyl]piperazin-1- yl}ethyl)phenoxy]-N-[(1-ethylpyrrolidin-2- yl)methyl]butan-1-amine | 589 |
| 518 | 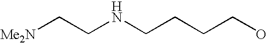 | N′-{4-[2,3-dimethyl-4-(1-{4-[4- (trifluoromethyl)benzoyl]piperazin-1- yl}ethyl)phenoxy]butyl}-N,N-dimethylethane- 1,2-diamine | 549 |
| 519 | 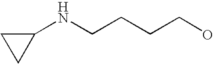 | N-{4-[2,3-dimethyl-4-(1-{4-[4- (trifluoromethyl)benzoyl]piperazin-1- yl}ethyl)phenoxy]butyl}cyclopropanamine | 518 |
| 520 | 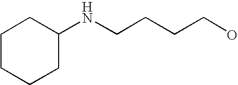 | N-{4-[2,3-dimethyl-4-(1-{4-[4- (trifluoromethyl)benzoyl]piperazin-1- yl}ethyl)phenoxy]butyl}cyclohexanamine | 560 |
| 521 | 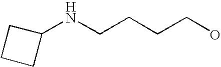 | N-{4-[2,3-dimethyl-4-(1-{4-[4- (trifluoromethyl)benzoyl]piperazin-1- yl}ethyl)phenoxy]butyl}cyclobutanamine | 532 |
| 522 | 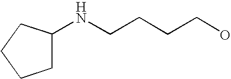 | N-{4-[2,3-dimethyl-4-(1-{4-[4- (trifluoromethyl)benzoyl]piperazin-1- yl}ethyl)phenoxy]butyl}cyclopentanamine | 546 |
| 523 | 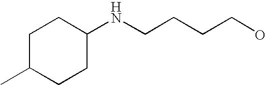 | N-{4-[2,3-dimethyl-4-(1-{4-[4- (trifluoromethyl)benzoyl]piperazin-1- yl}ethyl)phenoxy]butyl}-4- methylcyclohexanamine | 574 |
| 524 | 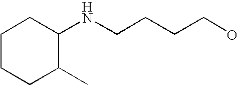 | N-{4-[2,3-dimethyl-4-(1-{4-[4- (trifluoromethyl)benzoyl]piperazin-1- yl}ethyl)phenoxy]butyl}-2- methylcyclohexanamine | 574 |
| 525 | 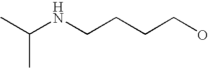 | 4-[2,3-dimethyl-4-(1-{4-[4- (trifluoromethyl)benzoyl]piperazin-1- yl}ethyl)phenoxy]-N-isopropylbutan-1-amine | 520 |
| 526 | 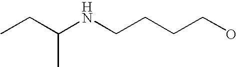 | N-(sec-butyl)-4-[2,3-dimethyl-4-(1-{4-[4- (trifluoromethyl)benzoyl]piperazin-1- yl}ethyl)phenoxy]butan-1-amine | 534 |
| 527 | 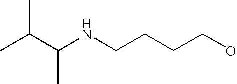 | N-(1,2-dimethylpropyl)-4-[2,3-dimethyl-4-(1- {4-[4-(trifluoromethyl)benzoyl]piperazin-1- yl}ethyl)phenoxy]butan-1-amine | 548 |
| 528 | 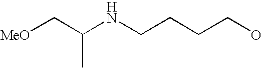 | 4-[2,3-dimethyl-4-(1-{4-[4- (trifluoromethyl)benzoyl]piperazin-1- yl}ethyl)phenoxy]-N-(2-methoxy-1- methylethyl)butan-1-amine | 550 |
| 529 | 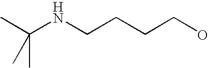 | N-(tert-butyl)-4-[2,3-dimethyl-4-(1-{4-[4- (trifluoromethyl)benzoyl]piperazin-1- yl}ethyl)phenoxy]butan-1-amine | 534 |
| 530 | 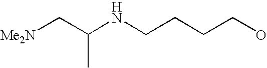 | N²-{4-[2,3-dimethyl-4-(1-{4-[4- (trifluoromethyl)benzoyl]piperazin-1- yl}ethyl)phenoxy]butyl}-N¹,N¹- dimethylpropane-1,2-diamine | 563 |
| 531 | 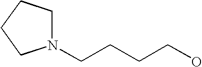 | 1-{1-[2,3-dimethyl-4-(4-pyrrolidin-1- ylbutoxy)phenyl]ethyl}-4-[4- (trifluoromethyl)benzoyl]piperazine | 532 |
| 532 | 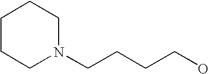 | 1-{1-[2,3-dimethyl-4-(4-piperidin-1- ylbutoxy)phenyl]ethyl}-4-[4- (trifluoromethyl)benzoyl]piperazine | 546 |
| 533 | 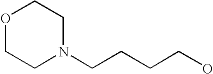 | 4-{4-[2,3-dimethyl-4-(1-{4-[4- (trifluoromethyl)benzoyl]piperazin-1- morpholine | 548 |
| 534 | 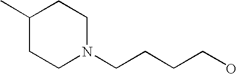 | 1-(1-{2,3-dimethyl-4-[4-(4-methylpiperidin-1- yl)butoxy]phenyl}ethyl)-4-[4- (trifluoromethyl)benzoyl]piperazine | 560 |
| 535 | 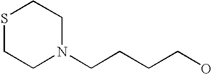 | 4-{4-[2,3-dimethyl-4-(1-{4-[4- (trifluoromethyl)benzoyl]piperazin-1- yl}ethyl)phenoxy]butyl}thiomorpholine | 564 |
| 536 | 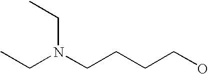 | 4-[2,3-dimethyl-4-(1-{4-[4- (trifluoromethyl)benzoyl]piperazin-1- yl}ethyl)phenoxy]-N,N-diethylbutan-1-amine | 534 |
| 537 | 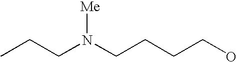 | 4-[2,3-dimethyl-4-(1-{4-[4- (trifluoromethyl)benzoyl]piperazin-1- yl}ethyl)phenoxy]-N-methyl-N-propylbutan-1- amine | 534 |
| 538 | 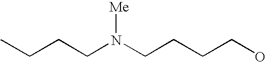 | N-butyl-4-[2,3-dimethyl-4-(1-{4-[4- (trifluoromethyl)benzoyl]piperazin-1- yl}ethyl)phenoxy]-N-methylbutan-1-amine | 548 |
| 539 | 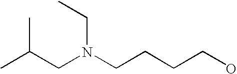 | 4-[2,3-dimethyl-4-(1-{4-[4- (trifluoromethyl)benzoyl]piperazin-1- yl}ethyl)phenoxy]-N-ethyl-N-isopropylbutan-1- amine | 548 |
| 540 | 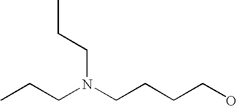 | 4-[2,3-dimethyl-4-(1-{4-[4- (trifluoromethyl)benzoyl]piperazin-1- yl}ethyl)phenoxy]-N,N-dipropylbutan-1-amine | 562 |
| 541 | 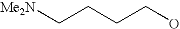 | N-{2-p8 2,3-dimethyl-4-{4-[4- (trifluoromethyl)benzoyl]piperazin-1- yl}ethyl)phenoxy]-N,N-dimethylbutan-1-amine | 506 |
| 542 | 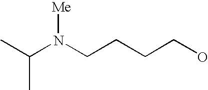 | 4-[2,3-dimethyl-4-(1-{4-[4- (trifluoromethyl)benzoyl]piperazin-1- yl}ethyl)phenoxy]-N-isopropyl-N-methylbutan- 1-amine | 534 |
| 543 | 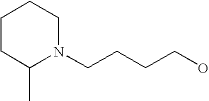 | 1-(1-{2,3-dimethyl-4-[4-(2-methylpiperidin-1- yl)butoxy]phenyl}ethyl)-4-[4- (trifluoromethyl)benzoyl]piperazine | 560 |
| 544 | 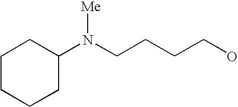 | N-{4-[2,3-dimethyl-4-(1-{4-[4- (trifluoromethyl)benzoyl]piperazin-1- yl}ethyl)phenoxy]butyl}-N- methylcyclohexanamine | 574 |
| 545 | 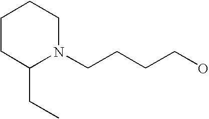 | 1-(1-{4-[4-(2-ethylpiperidin-1-yl)butoxy]-2,3- dimethylphenyl}ethyl)-4-[4- (trifluoromethyl)benzoyl]piperazine | 574 |
| 546 | 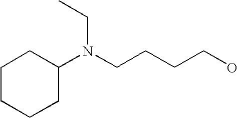 | N-{4-[2,3-dimethyl-4-(1-{4-[4- (trifluoromethyl)benzoyl]piperazin-1- yl}ethyl)phenoxy]butyl}-N- ethylcyclohexanamine | 588 |
| 547 | 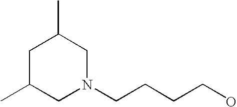 | 1-(1-{4-[4-(3,5-dimethylpiperidin-1-yl)butoxy]- 2,3-dimethylphenyl}ethyl)-4-[4- (trifluoromethyl)benzoyl]piperazine | 574 |
| 548 | 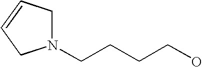 | 1-(1-{4-[4-(2,5-dihydro-1H-pyrrol-1- yl)butoxy]-2,3-dimethylphenyl}ethyl)-4-[4- (trifluoromethyl)benzoyl]piperazine | 530 |
| 549 | 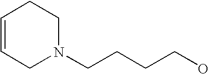 | 1-(1-{4-[4-(3,6-dihydropyridin-1(2H)- yl)butoxy]-2,3-dimethylphenyl}ethyl)-4-[4- (trifluoromethyl)benzoyl]piperazine | 544 |
| 550 | 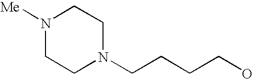 | 1-(1-{2,3-dimethyl-4-[4-(4-methylpiperazin-1- yl)butoxy]phenyl}ethyl)-4-[4- (trifluoromethyl)benzoyl]piperazine | 561 |
| 551 | 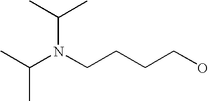 | 4-[2,3-dimethyl-4-(1-{4-[4- (trifluoromethyl)benzoyl]piperazin-1- yl}ethyl)phenoxy]-N,N-diisopropylbutan-1- amine | 562 |
| 552 | 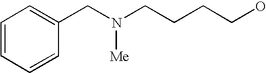 | N-benzyl-4-[2,3-dimethyl-4-(1-{4-[4- (trifluoromethyl)benzoyl]-piperazin-1- yl}ethyl)phenoxy]-N-methylbutan-1-amine | 582 |

TABLE III

| | COMPOUND | NAME | MS |
|---|---|---|---|
| 553 | 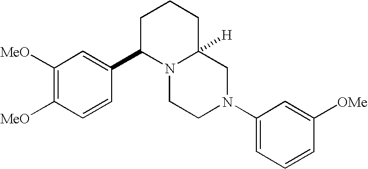 | (6R,9aS)-6-(3,4-dimethoxy-phenyl)- 2-(3-methoxyphenyl)octa-hydro-2H- pyrido[1,2-a]pyrazine | 383 |
| 554 | 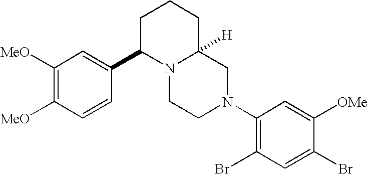 | (6R,9aS)-2-(2,4-dibromo-5- dimethoxyphenyDoctahydro-2H- pyrido[1,2-a]pyrazine | 539 |
| 555 | 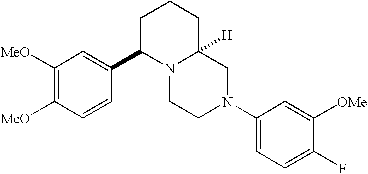 | 6-(3,4-dimethoxyphenyl)-2-(4-fluoro- 3-methoxyphenyl)octahydro-2H- pyrido[1,2-a]pyrazine | 401 |
| 556 | 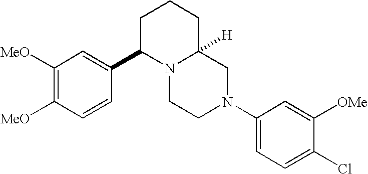 | 2-(4-chloro-3-methoxyphenyl)-6- (3,4-dimethoxyphenyl)octahydro-2H- pyrido[1,2-a]pyrazine | 417 |
| 557 | 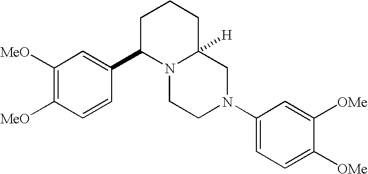 | 2,6-bis(3,4-dimethoxy- phenyl)octahydro-2H-pyrido- [1,2-a]pyrazine | 413 |
| 558 | 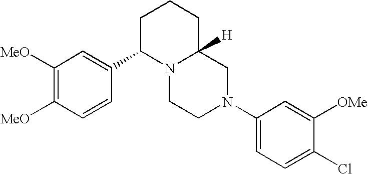 | (6S,9aR)-2-(4-chloro-3- methoxyphenyl)-6-(3,4- dimethoxyphenyl)octahydro-2H- pyrido[1,2-a]pyrazine | 417 |
| 559 | 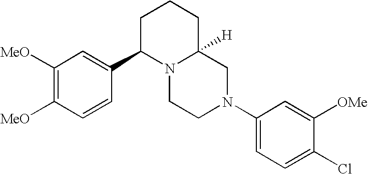 | (6R,9aS)-2-(4-chloro-3- methoxyphenyl)-6-(3,4- dimethoxyphenyl)octahydro-2H- pyrido[1,2-a]pyrazine | 417 |
| 560 | 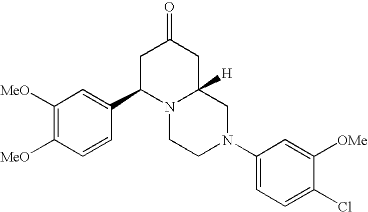 | (6R,9aR)-2-(4-chloro-3- methoxyphenyl)-6-(3,4- dimethoxyphenyl)octahydro-8H- pyrido[1,2-a]pyrazin-8-one | 431 |
| 561 | 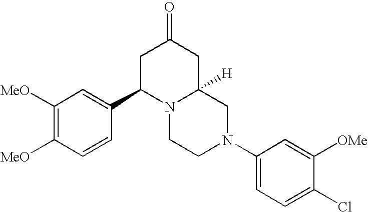 | (6R,9aS)-2-(4-chloro-3- methoxyphenyl)-6-(3,4- pyrido[1,2-a]pyrazin-8-one | 431 |
| 562 | 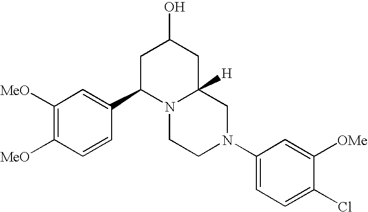 | (6R,9aR)-2-(4-chloro-3- methoxyphenyl)-6-(3,4- dimethoxyphenyl)octahydro-2H- pyrido[1,2-a]pyrazin-8-ol | 433 |
| 563 | 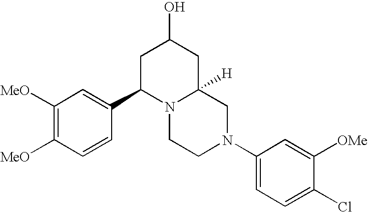 | (6R,9aS)-2-(4-chloro-3- methoxyphenyl)-6-(3,4- dimethoxyphenyl)octahydro-2H- pyrido[1,2-a]pyrazin-8-ol | 433 |
| 564 | 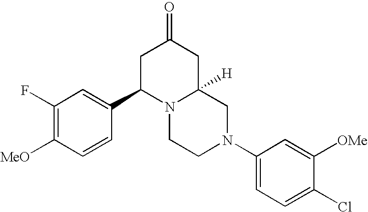 | (6R,9aS)-2-(4-chloro-3- methoxyphenyl)-6-(3-fluoro-4- methoxyphenyl)octahydro-8H- pyrido[1,2-a]pyrazin-8-one | 419 |
| 565 | 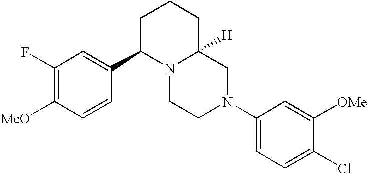 | (6R,9aS)-2-(4-chloro-3- methoxyphenyl)-6-(3-fluoro-4- methoxyphenyl)octahydro-2H- pyrido[1,2-a]pyrazine | 405 |
| 566 | 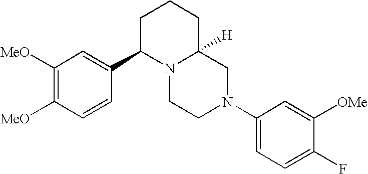 | (6R,9aS)-6-(3,4-dimethoxyphenyl)-2- (4-fluoro-3- methoxyphenyl)octahydro-2H- pyrido[1,2-a]pyrazine | 401 |
| 567 | 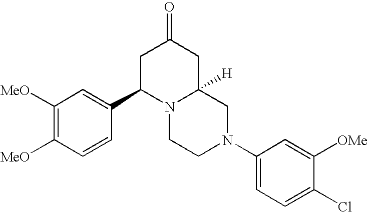 | (6R,9aS)-2-(4-chloro-3- methoxyphenyl)-6-(3,4- dimethoxyphenyl)octahydro-8H- pyrido[1,2-a]pyrazin-8-one | 431 |
| 568 | 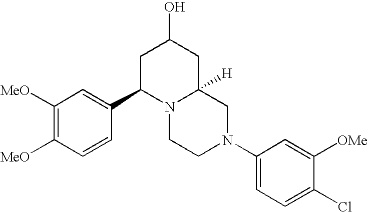 | (6R,9aS)-2-(4-chloro-3- methoxyphenyl)-6-(3,4- dimethoxyphenyl)octahydro-2H- pyrido[1,2-a]pyrazin-8-ol | 433 |
| 569 | 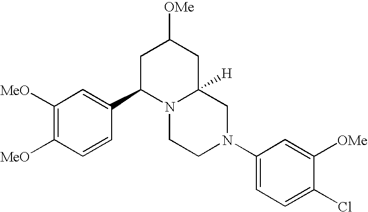 | (6R,9aS)-2-(4-chloro-3- methoxyphenyl)-6-(3,4-dimethoxy- phenyl)-8-methoxyoctahydro-2H- pyrido[1,2-a]pyrazine | 447 |
| 570 | 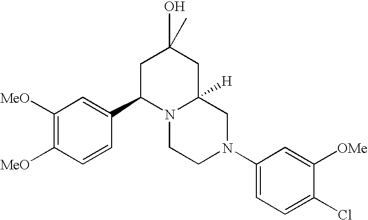 | (6R,8S,9aS)-2-(4-chloro-3- methoxyphenyl)-6-{3,4-dimethoxy- phenyl)-8-methyloctahydro-2H- pyrido[1,2-a]pyrazin-8-ol | 447 |
| 571 | 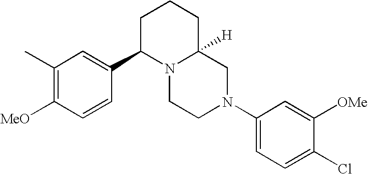 | (6R,9aS)-2-(4-chloro-3- methoxyphenyl)-6-(4-methoxy-3- methylphenyl)octahydro-2H- pyrido[1,2-a]pyrazine | 401 |
| 572 | 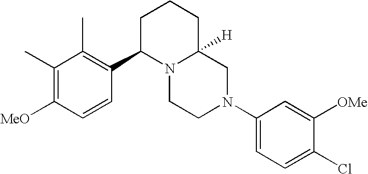 | (6R,9aS)-2-(4-chloro-3- methoxyphenyl)-6-(4-methoxy-2,3- dimethylphenyl)octahydro-2H- pyrido[1,2-a]pyrazine | 415 |
| 573 | 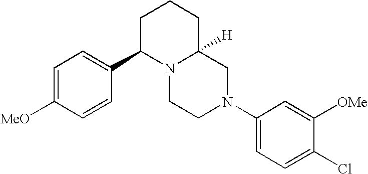 | (6R,9aS)-2-(4-chloro-3- methoxyphenyl)-6-(4- methoxyphenyl)octahydro-2H- pyrido[1,2-a]pyrazine | 387 |
| 574 | 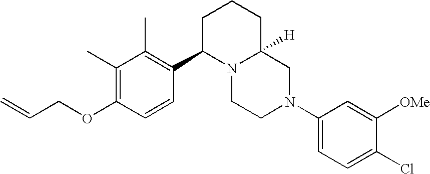 | (6R,9aS)-6-[4-(allyloxy)-2,3- dimethylphenyl]-2-(4-chloro-3- methoxyphenyl)octahydro-2H- pyrido[1,2-a]pyrazine | 441 |
| 575 | 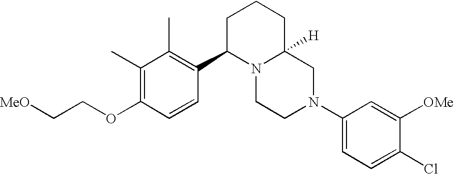 | (6R,9aS)-2-(4-chloro-3- methoxyphenyl)-6-[4-(2- methoxyethoxy)-2,3- pyrido[1,2-a]pyrazine | 459 |
| 576 | 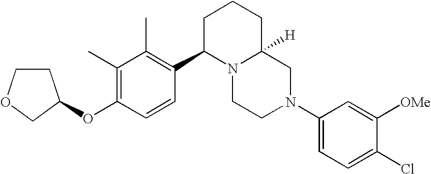 | (6R,9aS)-2-(4-chloro-3- methoxyphenyl)-6-{2,3-dimethyl-4- [(3R)-tetrahydrofuran-3- yloxy]phenyl}octahydro-2H- pyrido[1,2-a]pyrazine | 471 |
| 577 | 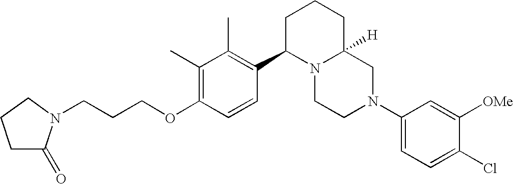 | 1-(3-{4-[(6R,9aS)-2-(4-chloro-3- methoxyphenyl)octahydro-2H- pyrido[1,2-a]pyrazin-6-yl]-2,3- dimethylphenoxy}propyl)-pyrrolidin- 2-one | 526 |
| 578 | 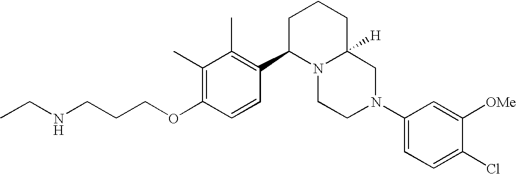 | 3-{4-[(6R,9aS)-2-(4-chloro-3- methoxyphenyl)octahydro-2H- pyrido[1,2-a]pyrazin-6-yl]-2,3- dimethylphenoxy}-N-ethylpropan-1- amine | 486 |
| 579 | 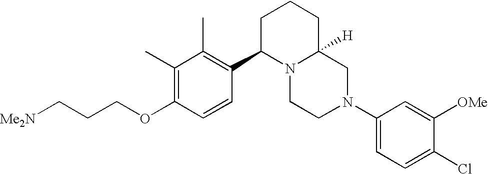 | 3-{4-[(6R,9aS)-2-(4-chloro-3- methoxyphenyl)octahydro-2H- pyrido[1,2-a]pyrazin-6-yl]-2,3- dimethylphenoxy}-N,N- dimethylpropan-1-amine | 486 |
| 580 | 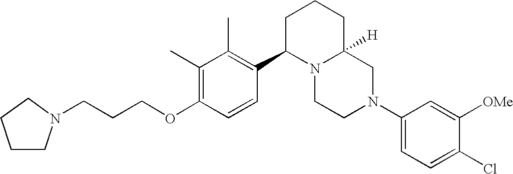 | (6R,9aS)-2-(4-chloro-3- methoxyphenyl)-6-[2,3-dimethyl-4- (3-pyrrolidin-1- ylpropoxy)phenyl]octahydro-2H- pyrido[1,2-a]pyrazine | 512 |
| 581 | 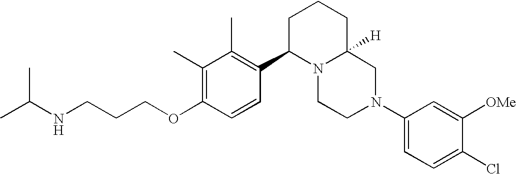 | 3-{4-[(6R,9aS)-2-(4-chloro-3- methoxyphenyl)octahydro-2H- pyrido[1,2-a]pyrazin-6-yl]-2,3- dimethylphenoxy}-N- isopropylpropan-1-amine | 500 |
| 582 | 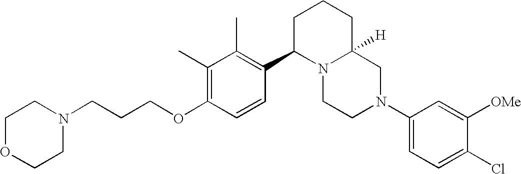 | (6R,9aS)-2-(4-chloro-3- methoxyphenyl)-6-[2,3-dimethyl-4- (3-morpholin-4- ylpropoxy)phenyl]octahydro-2H- pyrido[1,2-a]pyrazine | 528 |
| 583 | 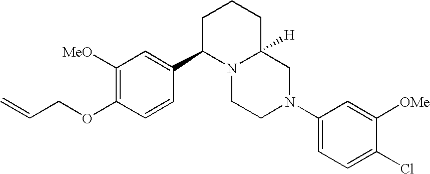 | (6R,9aS)-6-[4-(allyloxy)-3- methoxyphenyl]-2-(4-chloro-3- methoxyphenyl)octahydro-2H- pyrido[1,2-a]pyrazine | 443 |
| 584 | 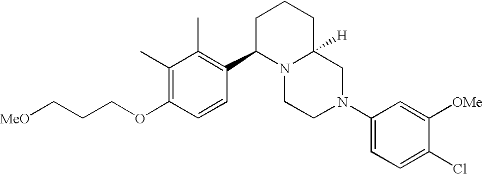 | (6R,9aS)-2-(4-chloro-3- methoxyphenyl)-6-[4-(3- methoxypropoxy)-2,3- dimethylphenyl]octahydro-2H- pyrido[1,2-a]pyrazine | 473 |
| 585 | 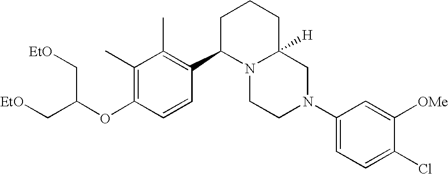 | (6R,9aS)-2-(4-chloro-3- methoxyphenyl)-6-{4-[2-ethoxy-1- (ethoxymethyl)ethoxy]-2,3- dimethylphenyl}octahydro-2H- pyrido[1,2-a]pyrazine | 531 |
| 586 | 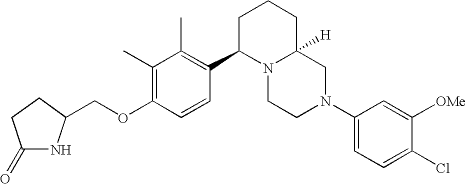 | 5-({4-[(6R,9aS)-2-(4-chloro-3- methoxyphenyl)octahydro-2H- pyrido[1,2-a]pyrazin-6-yl]-2,3- dimethylphenoxy}methyl)-pyrrolidin- 2-one | 498 |
| 587 | 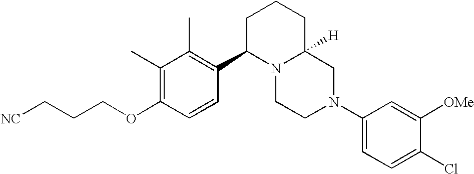 | 4-{4-[(6R,9aS)-2-(4-chloro-3- methoxyphenyl)octahydro-2H- pyrido[1,2a]pyridin-6-yl]-2,3- dimethylphenoxy}butanenitrile | 468 |
| 588 | 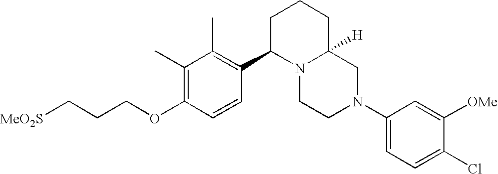 | (6R,9aS)-2-(4-chloro-3- methoxyphenyl)-6-{2,3-dimethyl-4- [3-(methylsulfonyl)propoxy]- phenyl}octahydro-2H-pyrido- [1,2-a]pyrazine | 521 |
| 589 | 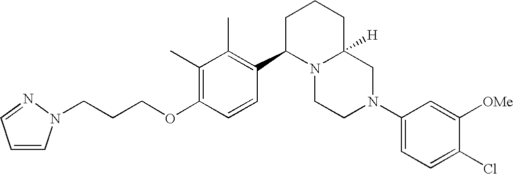 | (6R,9aS)-2-(4-chloro-3- methoxyphenyl)-6-{2,3-dimethyl-4- [3-(1H-pyrazol-1- yl)propoxy]phenyl}octahydro-2H- pyrido[1,2-a]pyrazine | 509 |
| 590 | 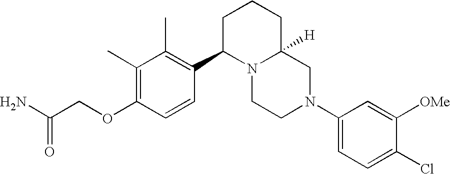 | 2-{4-[(6R,9aS)-2-(4-chloro-3- methoxyphenyl)octahydro-2H- pyrido[1,2-a]pyrazin-6-yl]-2,3- dimethylphenoxy}acetamide | 458 |
| 591 | 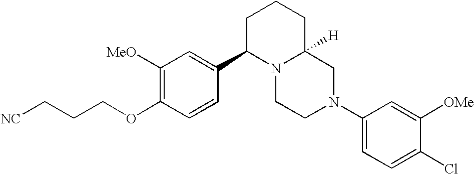 | 4-{4-[(6R,9aS)-2-(4-chloro-3- methoxyphenyl)octahydro-2H- pyrido[1,2-a]pyrazin-6-yl]-2- methoxyphenoxy}butanenitrile | 470 |
| 592 | 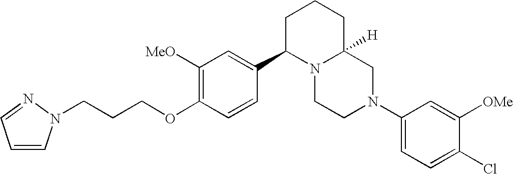 | (6R,9aS)-2-(4-chloro-3- methoxyphenyl)-6-{3-methoxy-4-[3- (1H-pyrazol-1- yl)propoxy]phenyl}octahydro-2H- pyrido[1,2-a]pyrazine | 511 |
| 593 | 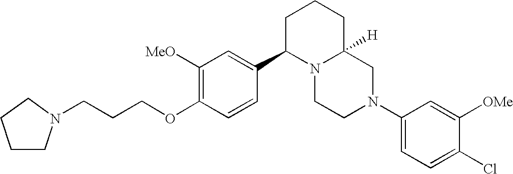 | (6R,9aS)-2-(4-chloro-3- methoxyphenyl)-6-[3-methoxy-4-(3- pyrrolidin-1- ylpropoxy)phenyl]octahydro-2H- pyrido[1,2-a]pyrazine | 514 |
| 594 | 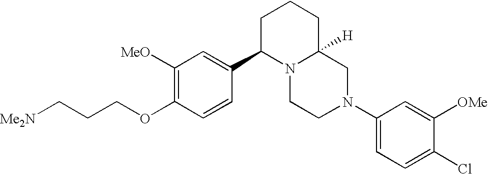 | 3-{4-[(6R,9aS)-2-(4-chloro-3- methoxyphenyl)octahydro-2H- pyrido[1,2-a]pyrazin-6-yl]-2- methoxyphenoxy}-N,N- dimethylpropan-1-amine | 488 |
| 595 | 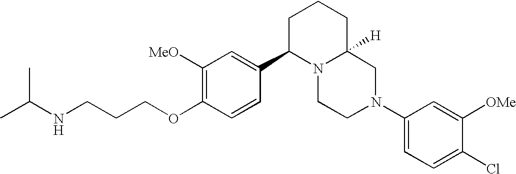 | 3-{4-[(6R,9aS)-2-(4-chloro-3- methoxyphenyl)octahydro-2H- pyrido[1,2-a]pyrazin-6-yl]-2- methoxyphenoxy}-N- isopropylpropan-1-amine | 502 |
| 596 | 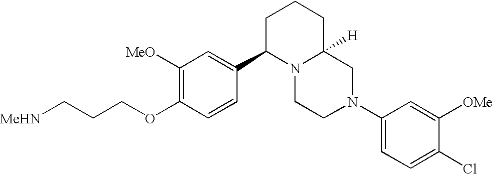 | {4-[(6R,9aS)-2-(4-chloro-3- methoxyphenyl)octahydro-2H- pyrido[1,2-a]pyrazin-6-yl]-2- methoxyphenoxy}-N-methylpropan- 1-amine | 474 |
| 597 | 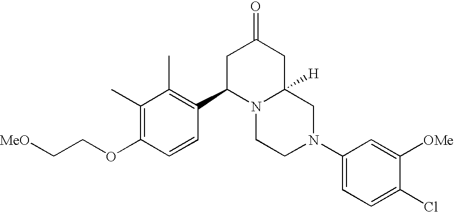 | (6R,9aS)-2-(4-chloro-3- methoxyphenyl)-6-[4-(2- methoxyethoxy)-2,3- dimethylphenyl]octahydro-8H- pyrido[1,2-a]pyrazin-8-one | 473 |
| 598 | 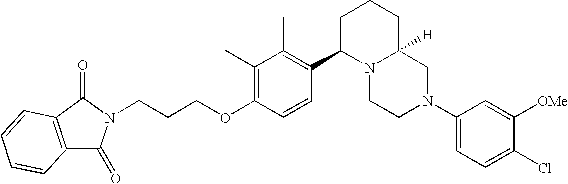 | 2-(3-{4-[(6R,9aS)-2-(4-chloro-3- methoxyphenyl)octahydro-2H- pyrido[1,2-a]pyrazin-6-yl]-2,3- dimethylphenoxy}propyl)-1H- isoindole-1,3(2H)-dione | 588 |
| 599 | 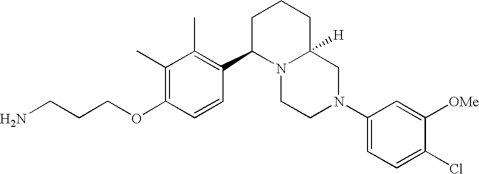 | 3-{4-[(6R,9aS)-2-(4-chloro-3- methoxyphenyl)octahydro-2H- pyrido[1,2-a]pyrazin-6-yl]-2,3- dimethylphenoxy}propan-1-amine | 458 |
| 600 | 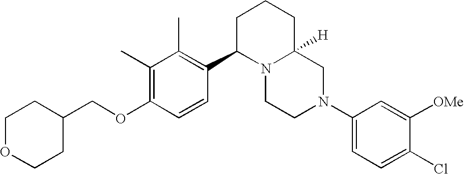 | (6R,9aS)-2-(4-chloro-3- methoxyphenyl)-6-[2,3-dimethyl-4- (tetrahydro-2H-pyran-4- ylmethoxy)phenyl]octahydro-2H- pyrido[1,2-a]pyrazine | 499 |
| 601 | 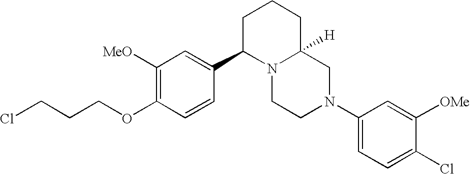 | (6R,9aS)-2-(4-chloro-3- methoxyphenyl)-6-[4-(3- chloropropoxy)-3- methoxyphenyl]octahydro-2H- pyrido[1,2-a]pyrazine | 479 |
| 602 | 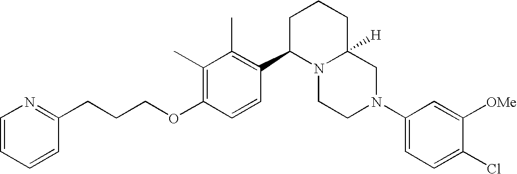 | (6R,9aS)-2-(4-chloro-3- methoxyphenyl)-6-[2,3-dimethyl-4- (3-pyridin-2-ylpropoxy)phenyl]- octahydro-2H-pyrido- [1,2-a]pyrazine | 520 |
| 603 | 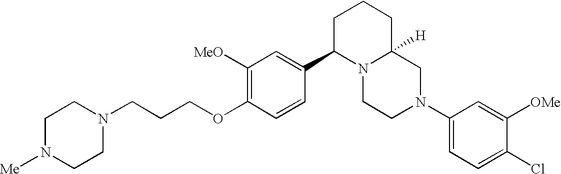 | (6R,9aS)-2-(4-chloro-3- methoxyphenyl)-6-(3-methoxy-4-[3- (4-methylpiperazin-1- yl)propoxy]phenyl}octahydro-2H- pyrido[1,2-a]pyrazine | 543 |
| 604 | 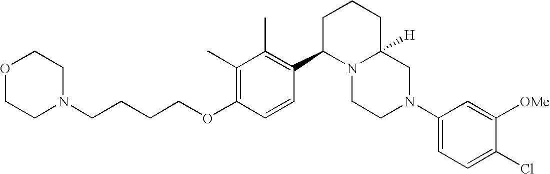 | (6R,9aS)-2-(4-chloro-3- methoxyphenyl)-6-[2,3-dimethyl-4- (4-morpholin-4- ylbutoxy)phenyl]octahydro-2H- pyrido[1,2-a]pyrazine | 542 |
| 605 | 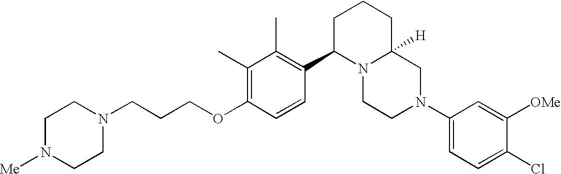 | (6R,9aS)-2-(4-chloro-3- methoxyphenyl)-6-{2,3-dimethyl-4- [3-(4-methylpiperazin-1- yl)propoxy]phenyl}octahydro-2H- pyrido[1,2-a]pyrazine | 541 |
| 606 | 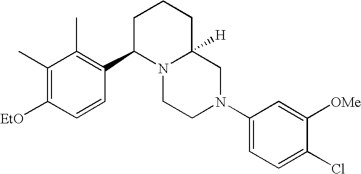 | (6R,9aS)-2-(4-chloro-3- methoxyphenyl)-6-(4-ethoxy-2,3- dimethylphenyl)octahydro-2H- pyrido[1,2-a]pyrazine | 429 |
| 607 | 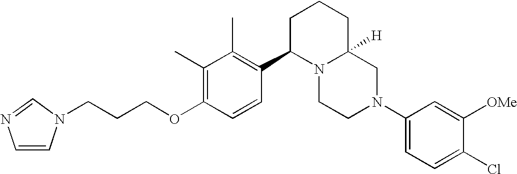 | (6R,9aS)-2-(4-chloro-3- methoxyphenyl)-6-{4-[3-(1H- imidazol-1-yl)propoxy]-2,3- dimethylphenyl}octahydro-2H- pyrido[1,2-a]pyrazine | 509 |
| 608 | 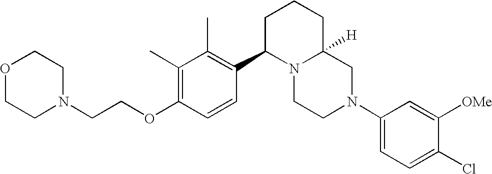 | (6R,9aS)-2-(4-chloro-3- methoxyphenyl)-6-[2,3-dimethyl-4- (2-morpholin-4- ylethoxy)phenyl]octahydro-2H- pyrido[1,2-a]pyrazine | 514 |
| 609 | 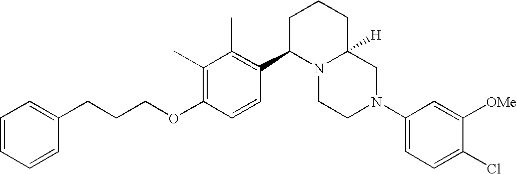 | (6R,9aS)-2-(4-chloro-3- methoxyphenyl)-6-[2,3-dimethyl-4- (3-phenylpropoxy)phenyl]octa-hydro- 2H-pyrido[1,2-a]pyrazine | 519 |
| 610 | 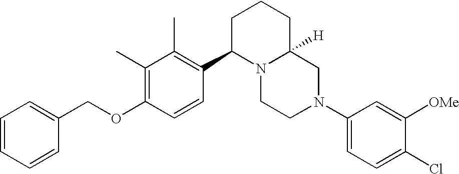 | (6R,9aS)-6-[4-(benzyloxy)-2,3- dimethylphenyl]-2-(4-chloro-3- methoxyphenyl)octahydro-2H- pyrido[1,2-a]pyrazine | 491 |
| 611 | 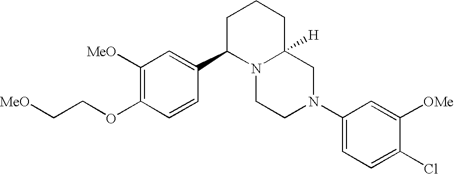 | (6R,9aS)-2-(4-chloro-3- methoxyphenyl)-6-[3-methoxy-4-(2- methoxyethoxy)phenyl]octa-hydro- 2H-pyrido[1,2-a]pyrazine | 461 |
| 612 | 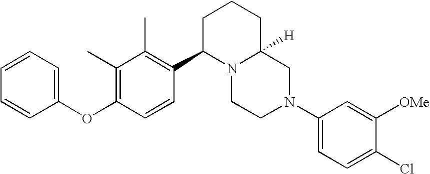 | (6R,9aS)-2-(4-chloro-3- methoxyphenyl)-6-(2,3-dimethyl-4- phenoxyphenyl)octahydro-2H- pyrido[1,2-a]pyrazine | 477 |
| 613 | 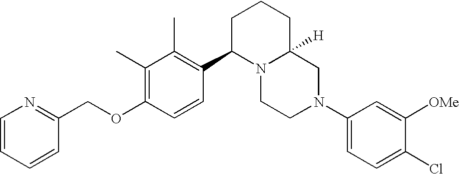 | (6R,9aS)-2-(4-chloro-3- methoxyphenyl)-6-[2,3-dimethyl-4- (pyridin-2- ylmethoxy)phenyl]octahydro-2H- pyrido[1,2-a]pyrazine | 492 |
| 614 | 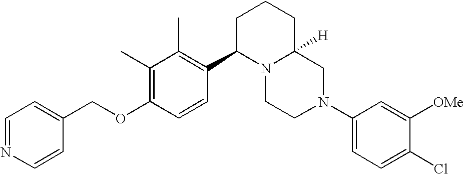 | (6R,9aS)-2-(4-chloro-3- methoxyphenyl)-6-[2,3-dimethyl-4- (pyridin-4- ylmethoxy)phenyl]octahydro-2H- pyrido[1,2-a]pyrazine | 492 |
| 615 | 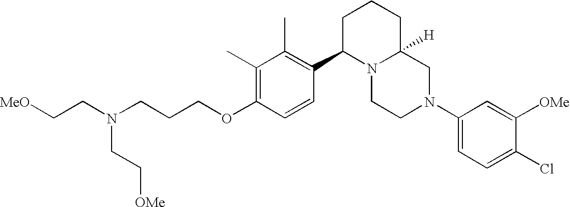 | 3-{4-[(6R,9aS)-2-(4-chloro-3- methoxyphenyl)octahydro-2H- dimethylphenoxy}-N,N-bis(2- methoxyethyl)propan-1-amine | 574 |
| 616 | 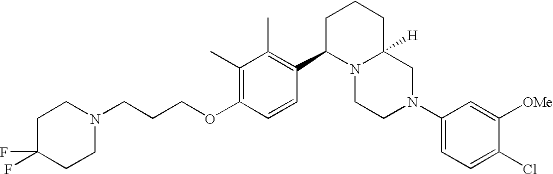 | (6R,9aS)-2-(4-chloro-3- methoxyphenyl)-6-{4-[3-(4,4- difluoropiperidin-1-yl)propoxy]-2,3- dimethylphenyl}octahydro-2H- pyrido[1,2-a]pyrazine | 562 |
| 617 | 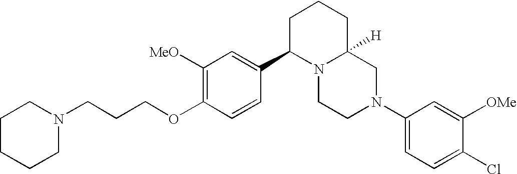 | (6R,9aS)-2-(4-chloro-3- methoxyphenyl)-6-[3-methoxy-4-(3- piperidin-1- ylpropoxy)phenyl]octahydro-2H- pyrido[1,2-a]pyrazine | 528 |
| 618 | 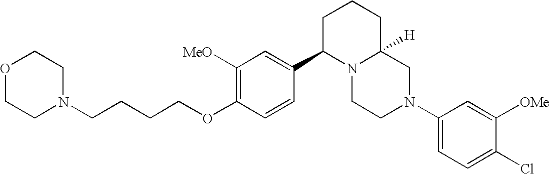 | (6R,9aS)-2-(4-chloro-3- morpholin-4- methoxyphenyl)-6-[3-methoxy-4-(4- ylbutoxy)phenyl]octahydro-2H- pyrido[1,2-a]pyrazine | 544 |
| 619 | 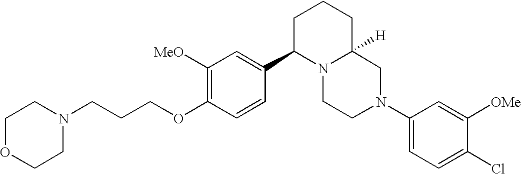 | (6R,9aS)-2-(4-chloro-3- methoxyphenyl)-6-[3-methoxy-4-(3- morpholin-4- ylpropoxy)phenyl]octahydro-2H- pyrido[1,2-a]pyrazine | 530 |
| 620 | 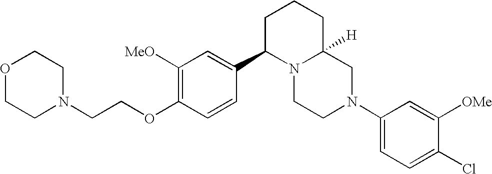 | (6R,9aS)-2-(4-chloro-3- methoxyphenyl)-6-[3-methoxy-4-(2- morpholin-4- ylethoxy)phenyl]octahydro-2H- pyrido[1,2-a]pyrazine | 516 |
| 621 | 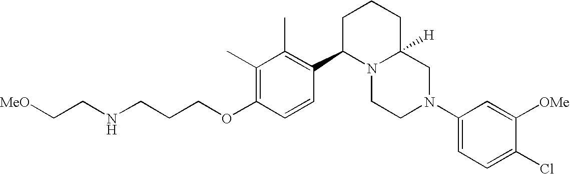 | 3-{4-[(6R,9aS)-2-(4-chloro-3- methoxyphenyl)octahydro-2H- pyrido[1,2-a]pyrazin-6-yl]-2,3- dimethylphenoxy}-N-(2- methoxyethyl)propan-1-amine | 516 |
| 622 | 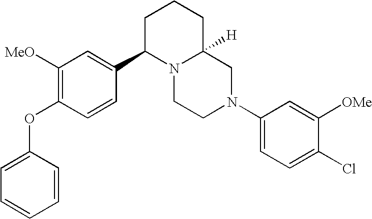 | (6R,9aS)-2-(4-chloro-3- methoxyphenyl)-6-(3-methoxy-4- phenoxyphenyl)octahydro-2H- pyrido[1,2-a]pyrazine | 479 |
| 623 | 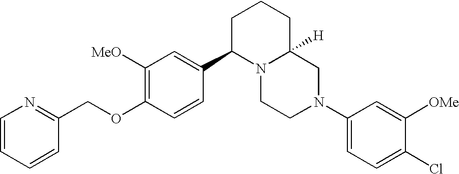 | (6R,9aS)-2-(4-chloro-3- methoxyphenyl)-6-[3-methoxy-4- (pyridin-2- ylmethoxy)phenyl]octahydro-2H- pyrido[1,2-a]pyrazine | 494 |
| 624 | 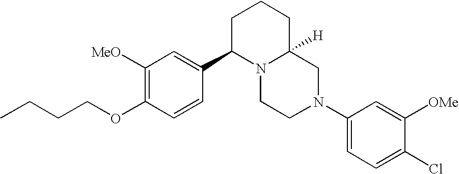 | (6R,9aS)-6-(4-butoxy-3- methoxyphenyl)-2-(4-chloro-3- methoxyphenyl)octahydro-2H- pyrido[1,2-a]pyrazine | 459 |
| 625 | 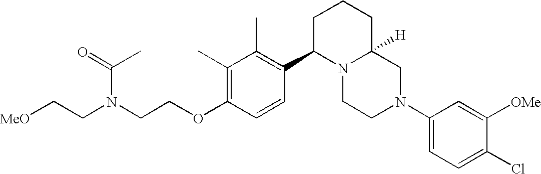 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-N-(2- methoxyethyl)acetamide | 558 |
| 626 | 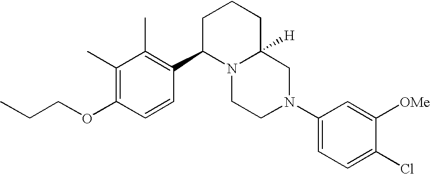 | (6R,9aS)-2-(4-chloro-3- methoxyphenyl)-6-(2,3-dimethyl-4- propoxyphenyl)octahydro-2H- pyrido[1,2-a]pyrazine | 443 |
| 627 | 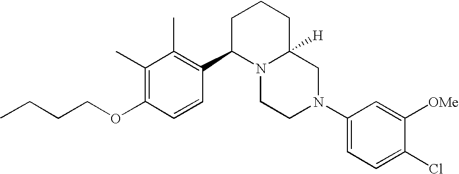 | (6R,9aS)-6-(4-butoxy-2,3- dimethylphenyl)-2-(4-chloro-3- methoxyphenyl)octahydro-2H- pyrido[1,2-a]pyrazine | 457 |
| 628 | 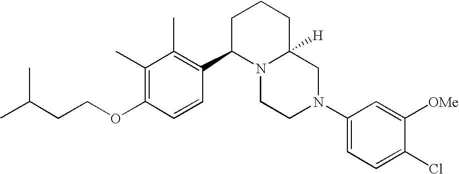 | (6R,9aS)-2-(4-chloro-3- methoxyphenyl)-6-[2,3-dimethyl-4- (3-methylbutoxy)phenyl]octahydro- 2H-pyrido[1,2-a]pyrazine | 471 |
| 629 | 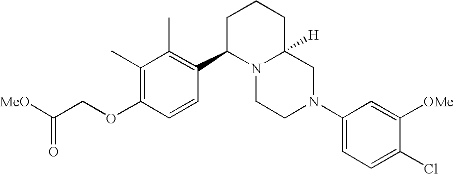 | methyl {4-[(6R,9aS)-2-(4-chloro-3- methoxyphenyl)octahydro-2H- pyrido[1,2-a]pyrazin-6-yl]-2,3- dimethylphenoxy}acetate | 473 |
| 630 | 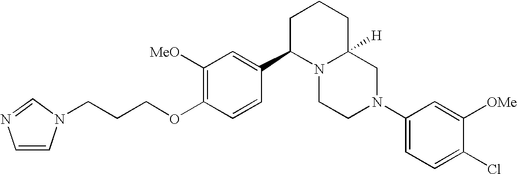 | (6R,9aS)-2-(4-chloro-3- methoxyphenyl)-6-{4-[3-(1H- imidazol-1-yl)propoxy]-3- methoxyphenyl}octahydro-2H- pyrido[1,2-a]pyrazine | 511 |
| 631 | 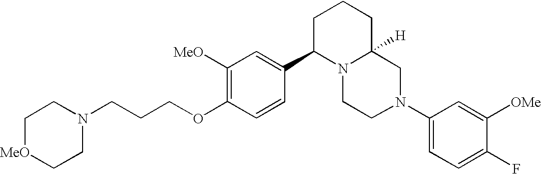 | (6R,9aS)-2-(4-fluoro-3- methoxyphenyl)-6-{3-methoxy-4-[3- (4-methylpiperazin-1- yl)propoxy]phenyl}octahydro-2H- pyrido[1,2-a]pyrazine | 527 |
| 632 | 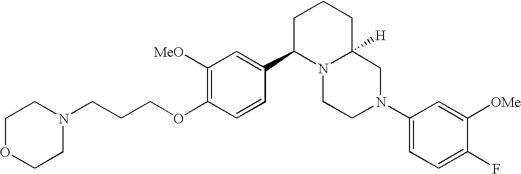 | (6R,9aS)-2-(4-fluoro-3- methoxyphenyl)-6-[3-methoxy-4-(3- morpholin-4- ylpropoxy)phenyl]octahydro-2H- pyrido[1,2-a]pyrazine | 514 |
| 633 | 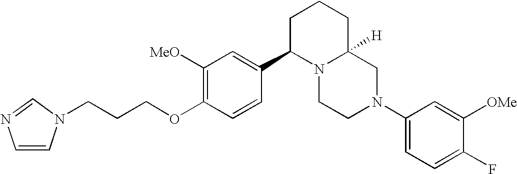 | (6R,9aS)-2-(4-fluoro-3- methoxyphenyl)-6-{4-[3-(1H- imidazol-1-yl)propoxy]-3- methoxyphenyl}octahydro-2H- pyrido[1,2-a]pyrazine | 495 |
| 634 | 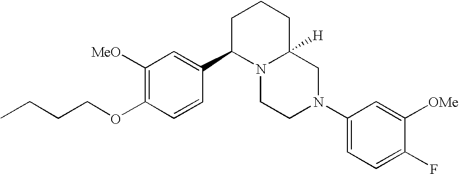 | (6R,9aS)-6-(4-butoxy-3- metboxyphenyl)-2-(4-fluoro-3- methoxyphenyl)octahydro-2H- pyrido[1,2-a]pyrazine | 443 |
| 635 | 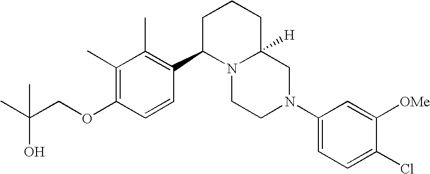 | 1-{4-[(6R,9aS)-2-(4-chloro-3- methoxyphenyl)octahydro-2H- pyrido[1,2-a]pyrazin-6-yl]-2,3- dimethylphenoxy}-2-methylpropan- 2-ol | 473 |
| 636 | 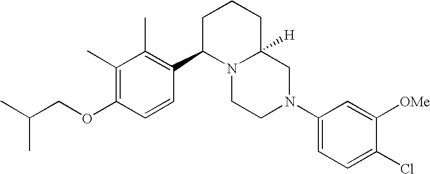 | (6R,9aS)-2-(4-chloro-3- methoxyphenyl)-6-(4-isobutoxy-2,3- dimethylphenyl)octahydro-2H- pyrido[1,2-a]pyrazine | 457 |
| 637 | 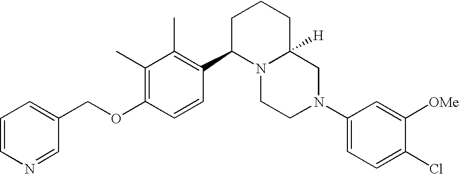 | (6R,9aS)-2-(4-chloro-3- methoxyphenyl)-6-[2,3-dimethyl-4- (pyridin-3-ylmethoxy)phenyl]octa- hydro-2H-pyrido[1,2-a]pyrazine | 492 |
| 638 | 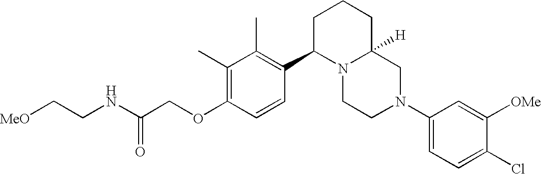 | 2-{4-[(6R,9aS)-2-(4-chloro-3- methoxyphenyl)octahydro-2H- pyrido[1,2-a]pyrazin-6-yl]-2,3- dimethylphenoxy}-N-(2- methoxyethyl)acetamide | 516 |
| 639 | 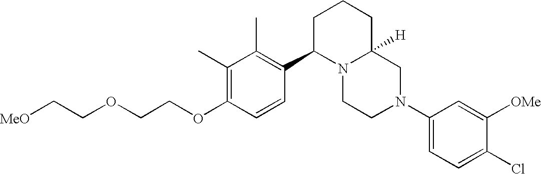 | (6R,9aS)-2-(4-chloro-3- methoxyphenyl)-6-{4-[2-(2- methoxyethoxy)ethoxy]-2,3- dimethylphenyl}octahydro-2H- pyrido[1,2-a]pyrazine | 503 |
| 640 | 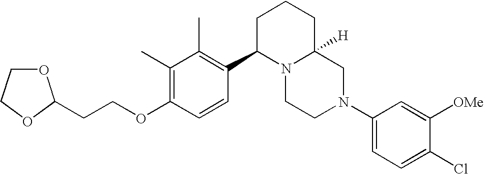 | (6R,9aS)-2-(4-chloro-3- methoxyphenyl)-6-{4-[2-(1,3- dioxolan-2-yl)ethoxy]-2,3- dimethylphenyl}octahydro-2H- pyrido[1,2-a]pyrazine | 501 |
| 641 | 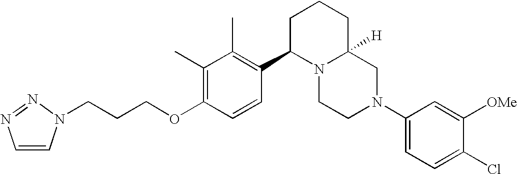 | (6R,9aS)-2-(4-chloro-3- methoxyphenyl)-6-{2,3-dimethyl-4- [3-(2H-1,2,3-triazol-2- yl)propoxy]phenyl}octahydro-2H- pyrido[1,2-a]pyrazine | 510 |
| 642 | 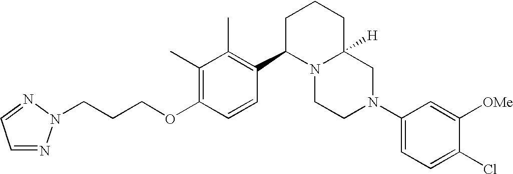 | (6R,9aS)-2-(4-chloro-3- methoxyphenyl)-6-{2,3-dimethyl-4- [3-(2H-1,2,3-triazol-2- yl)propoxy]phenyl}octahydro-2H- pyrido[1,2-a]pyrazine | 510 |
| 643 | 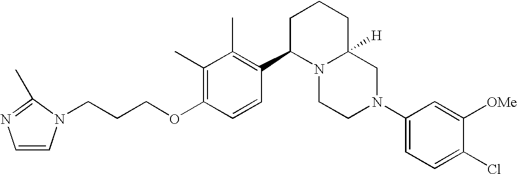 | (6R,9aS)-2-(4-chloro-3- methoxyphenyl)-6-{2,3-dimethyl-4- [3-(2-methyl-1H-imidazol-1- yl)propoxy]phenyl}octahydro-2H- pyrido[1,2-a]pyrazine | 523 |
| 644 | 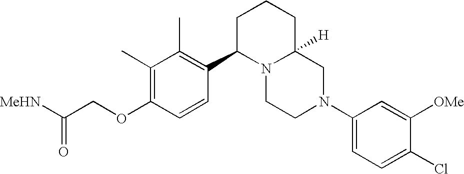 | 2-{4-[(6R,9aS)-2-(4-chloro-3- methoxyphenyl)octahydro-2H- pyrido[1,2-a]pyrazin-6-yl]-2,3- dimethylphenoxy}-N- methylacetamide | 472 |
| 645 | 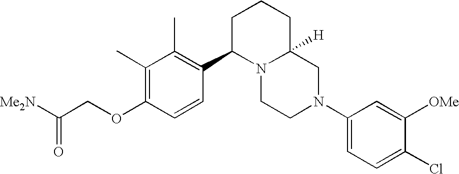 | 2-{4-[(6R,9aS)-2-(4-chloro-3- methoxyphenyl)octahydro-2H- pyrido[1,2-a]pyrazin-6-yl]-2,3- dimethylphenoxy}-N,N- dimethylacetamide | 486 |
| 646 | 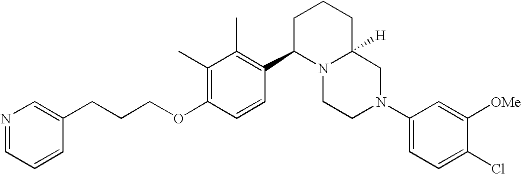 | (6R,9aS)-2-(4-chloro-3- methoxyphenyl)-6-[2,3-dimethyl-4- (3-pyridin-3-ylpropoxy)phenyl]- octahydro-2H-pyrido- [1,2-a]pyrazine | 520 |
| 647 | 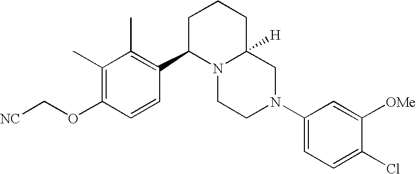 | {4-[(6R,9aS)-2-(4-chloro-3- pyrido[1,2-a]pyrazin-6-yl]-2,3- methoxyphenyl)octahydro-2H- dimethylphenoxy}acetonitrile | 440 |
| 648 | 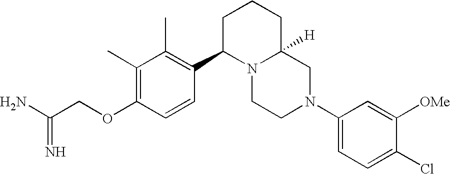 | 2-{4-[(6R,9aS)-2-(4-chloro-3- methoxyphenyl)octahydro-2H- pyrido[1,2-a]pyrazin-6-yl]-2,3- dimethylphenoxy}ethanimidamide | 457 |
| 649 | 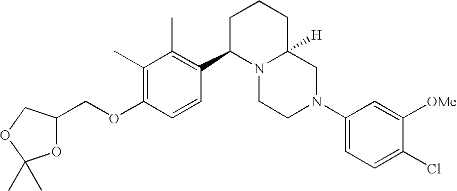 | (6R,9aS)-2-(4-chloro-3-methoxy- phenyl)-6-{4-[(2,2-dimethyl-1,3- dioxolan-4-yl)methoxy]-2,3- dimethylphenyl}-octahydro-2H- pyrido[1,2-a]pyrazine | 515 |
| 650 | 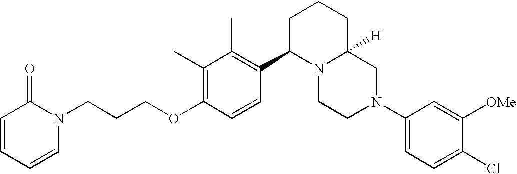 | 1-(3-{4-[(6R,9aS)-2-(4-chloro-3- methoxyphenyl)octahydro-2H- pyrido[1,2-a]pyrazin-6-yl]-2,3- dimethylphenoxy}propyl)pyridin- 2(1H)-one | 536 |
| 651 | 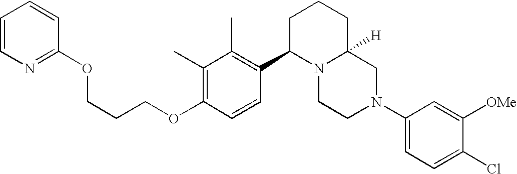 | (6R,9a5)-2-(4-chloro-3- methoxyphenyl)-6-{2,3-dimethyl-4- [3-(pyridin-2-yloxy)propoxy]- phenyl}octahydro-2H- pyrido[1,2-a]pyrazine | 536 |
| 652 | 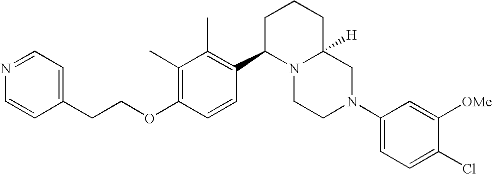 | (6R,9aS)-2-(4-chloro-3- methoxyphenyl)-6-[2,3-dimethyl-4- (2-pyridin-4-ylethoxy)phenyl]octa- hydro-2H-pyrido[1,2-a]pyrazine | 506 |
| 653 | 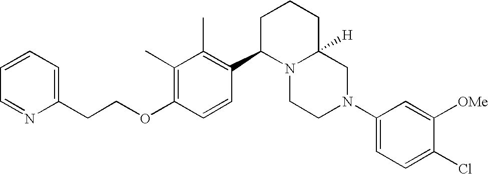 | (6R,9aS)-2-(4-chloro-3- methoxyphenyl)-6-[2,3-dimethyl-4- (2-pyridin-2-ylethoxy)phenyl]octa- hydro-2H-pyrido[1,2-a]pyrazine | 506 |
| 654 | 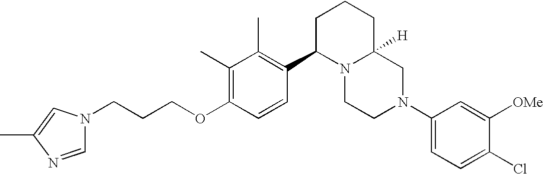 | (6R,9aS)-2-(4-chloro-3- methoxyphenyl)-6-{2,3-dimethyl-4- [3-(4-methyl-1H-imidazol-1- yl)propoxy]phenyl}octahydro-2H- pyrido[1,2-a]pyrazine | 523 |
| 655 | 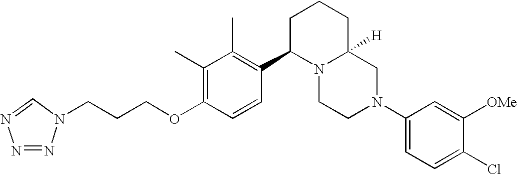 | (6R,9aS)-2-(4-chloro-3- methoxyphenyl)-6-{2,3-dimethyl-4- [3-(1H-tetrazol-1- yl)propoxy]phenyl}octahydro-2H- pyrido[1,2-a]pyrazine | 511 |
| 656 | 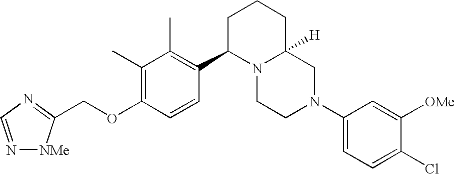 | (6R,9aS)-2-(4-chloro-3- methoxyphenyl)-6-{2,3-dimethyl-4- [(1-methyl-1H-1,2,4-triazol-5- yl)methoxy]phenyl}octahydro-2H- pyrido[1,2-a]pyrazine | 496 |
| 657 | 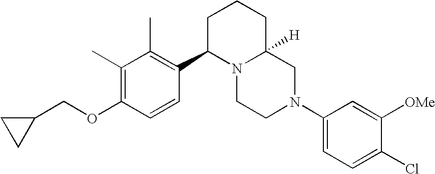 | (6R,9aS)-2-(4-chloro-3- methoxyphenyl)-6-[4- (cyclopropylmethoxy)-2,3- dimethylphenyl]octahydro-2H- pyrido[1,2-a]pyrazine | 455 |
| 658 | 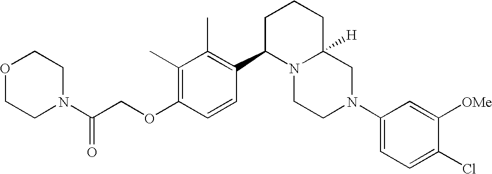 | (6R,9aS)-2-(4-chloro-3- methoxyphenyl)-6-[2,3-dimethyl-4- (2-morpholin-4-yl-2- oxoethoxy)phenyl]octahydro-2H- pyrido[1,2-a]pyrazine | 528 |
| 659 | 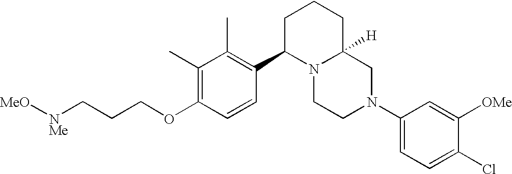 | 3-{4-[(6R,9aS)-2-(4-chloro-3- methoxyphenyl)octahydro-2H- pyrido[1,2-a]pyrazin-6-yl]-2,3- dimethylphenoxy}-N-methoxy-N- methylpropan-1-amine | 502 |
| 660 | 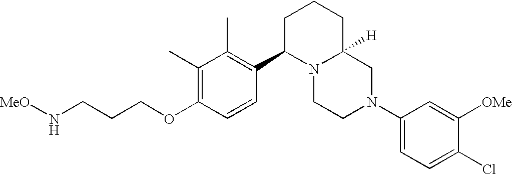 | 3-{4-[(6R,9aS)-2-(4-chloro-3- methoxyphenyl)octahydro-2H- pyrido[1,2-a]pyrazin-6-yl]-2,3- dimethylphenoxy}-N- methoxypropan-1-amine | 488 |
| 661 | 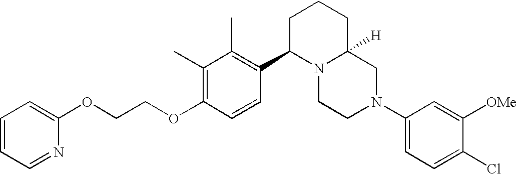 | (6R,9aS)-2-(4-chloro-3- methoxyphenyl)-6-{2,3-dimethyl-4- [2-(pyridin-2-yloxy)- ethoxy]phenyl}octahydro-2H- pyrido[1,2-a]pyrazine | 522 |
| 662 | 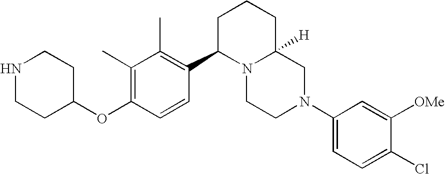 | (6R,9aS)-2-(4-chloro-3- methoxyphenyl)-6-[2,3-dimethyl-4- (piperidin-4-yloxy)phenyl]octa- hydro-2H-pyrido[1,2-a]pyrazine | 484 |
| 663 | 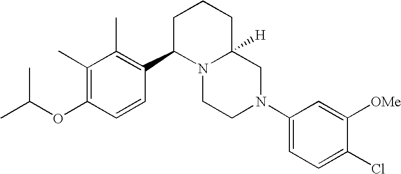 | (6R,9aS)-2-(4-chloro-3- methoxyphenyl)-6-(4-isopropoxy-2,3- dimethylphenyl)octahydro-2H- pyrido[1,2-a]pyrazine | 443 |
| 664 | 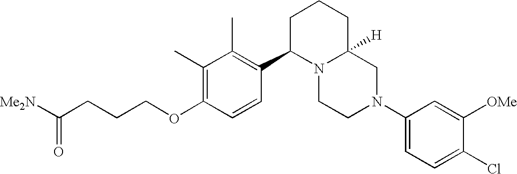 | 4-{4-[(6R,9aS)-2-(4-chloro-3- methoxyphenyl)octahydro-2H- pyrido[1,2-a]pyrazin-6-yl]-2,3- dimethylphenoxy}-N,N- dimethylbutanamide | 514 |
| 665 | 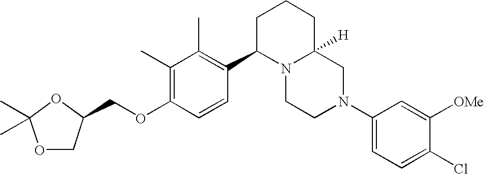 | (6R,9aS)-2-(4-chloro-3- methoxyphenyl)-6-(4-{[(4S)-2,2- dimethyl-1,3-dioxolan-4- dimethylphenyl)octahydro-2H- yl]methoxy}-2,3- pyrido[1,2-a]pyrazine | 515 |
| 666 | 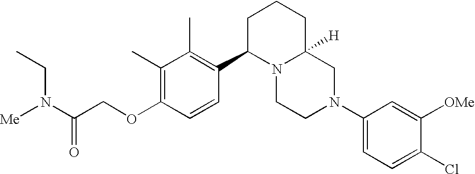 | 2-{4-[(6R,9aS)-2-(4-chloro-3- pyrido[1,2-a]pyrazin-6-yl]-2,3- methoxyphenyl)octahydro-2H- dimethylphenoxy}-N-ethyl-N- methylacetamide | 500 |
| 667 | 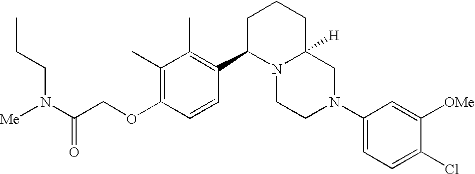 | 2-{4-[(6R,9aS)-2-(4-chloro-3- methoxyphenyl)octahydro-2H- pyrido[1,2-a]pyrazin-6-yl]-2,3- dimethylphenoxy}-N-methyl-N- propylacetamide | 514 |
| 668 | 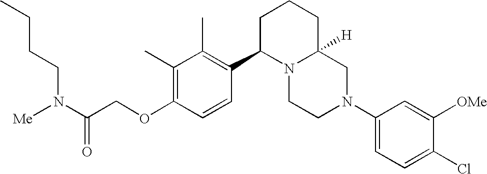 | N-butyl-2-{4-[(6R,9aS)-2-(4-chloro- 3-methoxyphenyl)octahydro-2H- pyrido[1,2-a]pyrazin-6-yl]-2,3- dimethylphenoxy}-N- methylacetamide | 528 |
| 669 | 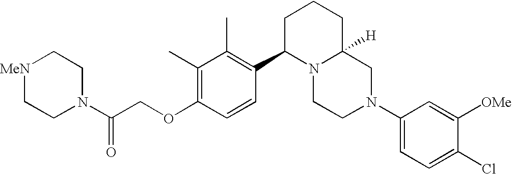 | (6R,9aS)-2-(4-chloro-3- methoxyphenyl)-6-{2,3-dimethyl-4- [2-(4-methylpiperazin-1-yl)-2- oxoethoxy]phenyl}octahydro-2H- pyrido[1,2-a]pyrazine | 541 |
| 670 | 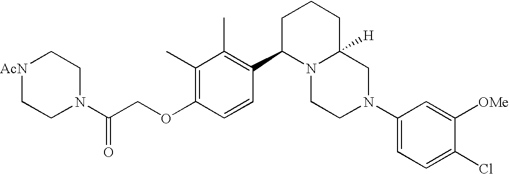 | (6R,9aS)-6-{4-[2-(4-acetylpiperazin- 1-yl)-2-oxoethoxy]-2,3- dimethylphenyl}-2-(4-chloro-3- methoxyphenyl)octahydro-2H- pyrido[1,2-a]pyrazine | 569 |
| 671 | 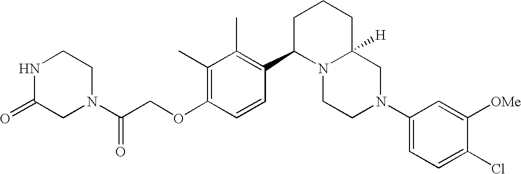 | 4-({4-[(6R,9aS)-2-(4-chloro-3- methoxyphenyl)octahydro-2H- pyrido[1,2-a]pyrazin-6-yl]-2,3- dimethylphenoxy}acetyl)piperazin-2- one | 541 |
| 672 | 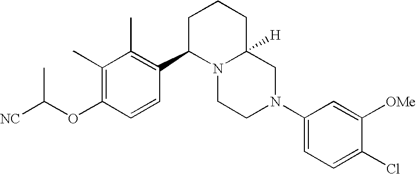 | 2-{4-[(6R,9aS)-2-(4-chloro-3- methoxyphenyl)octahydro-2H- pyrido[1,2-a]pyrazin-6-yl]-2,3- dimethylphenoxy}propanenitrile | 454 |
| 673 | 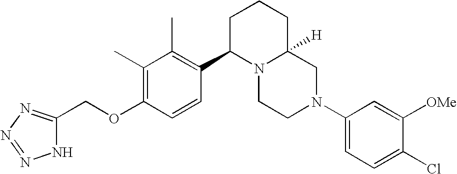 | (6R,9aS)-2-(4-chloro-3- methoxyphenyl)-6-[2,3-dimethyl-4- (1H-tetrazol-5- ylmethoxy)phenyl]octahydro-2H- pyrido[1,2-a]pyrazine | 483 |
| 674 | 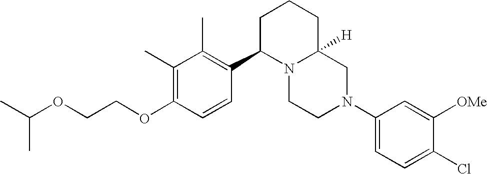 | (6R,9aS)-2-(4-chloro-3- methoxyphenyl)-6-[4-(2- isopropoxyethoxy)-2,3- dimethylphenyl]octahydro-2H- pyrido[1,2-a]pyrazine | 487 |
| 675 | 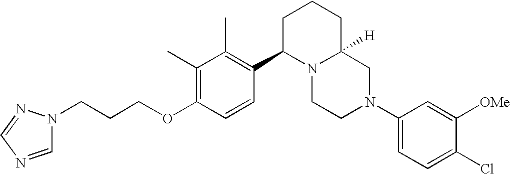 | (6R,9aS)-2-(4-chloro-3- methoxyphenyl)-6-{2,3-dimethyl-4- [3-(1H-1,2,4-triazol-1- yl)propoxy]phenyl}octahydro-2H- pyrido[1,2-a]pyrazine | 510 |
| 676 | 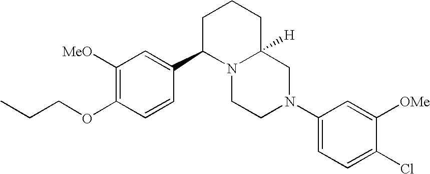 | (6R,9aS)-2-(4-chloro-3- methoxyphenyl)-6-(3-methoxy-4- propoxyphenyl)octahydro-2H- pyrido[1,2-a]pyrazine | 445 |
| 677 | 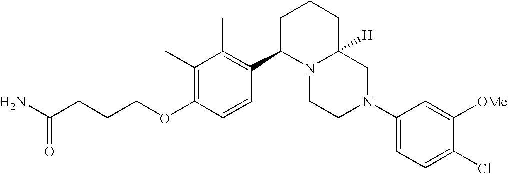 | 4-{4-[(6R,9aS)-2-(4-chloro-3- methoxyphenyl)octahydro-2H- pyrido[1,2-a]pyrazin-6-yl]-2,3- dimethylphenoxy}butanamide | 486 |
| 678 | 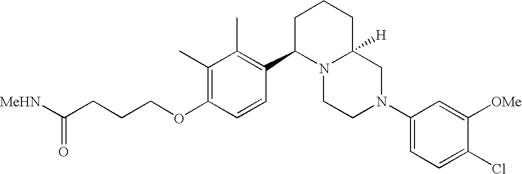 | 4-{4-[(6R,9aS)-2-(4-chloro-3- methoxyphenyl)octahydro-2H- pyrido[1,2-a]pyrazin-6-yl]-2,3- dimethylphenoxy}-N- methylbutanamide | 500 |
| 679 | 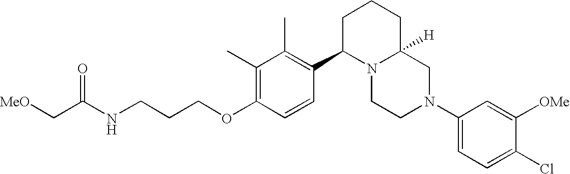 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-2- methoxyacetamide | 530 |
| 680 | 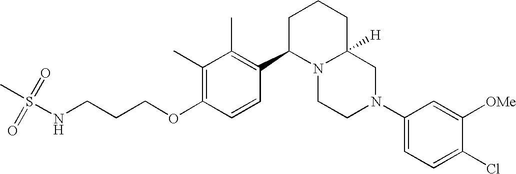 | N-(3-{4-[(6R,9aS)-2-(4-chloro-3- methoxyphenyl)octahydro-2H- pyrido[1,2-a]pyrazin-6-yl]-2,3- dimethylphenoxy}propyl)methane- sulfonamide | 536 |
| 681 | 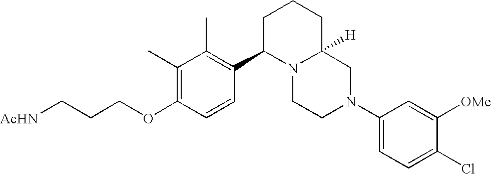 | N-(3-{4-[(6R,9aS)-2-(4-chloro-3- methoxyphenyl)octahydro-2H- pyrido[1,2-a]pyrazin-6-yl]-2,3- dimethylphenoxy}propyl)acetamide | 500 |
| 682 | 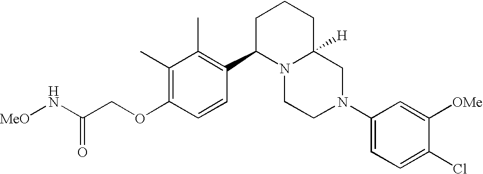 | 2-{4-[(6R,9aS)-2-(4-chloro-3- methoxyphenyl)octahydro-2H- pyrido[1,2-a]pyrazin-6-yl]-2,3- dimethylphenoxy}-N- methoxyacetamide | 488 |
| 683 | 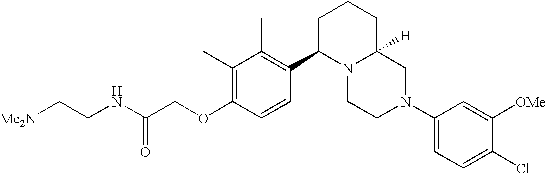 | 2-{4-[(6R,9aS)-2-(4-chloro-3- methoxyphenyl)octahydro-2H- pyrido[1,2-a]pyrazin-6-yl]-2,3- dimethylphenoxy}-N-[2- (dimethylamino)ethyl]acetamide | 529 |
| 684 | 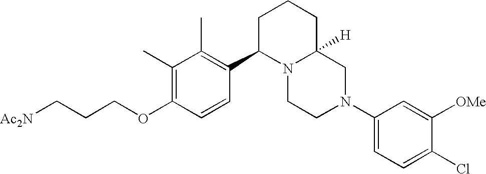 | N-Acetyl-N-(3-{4-[(6R,9aS)-2-(4- chloro-3-methoxy-phenyl)-octahydro- pyrido[1,2-a]pyrazin-6-yl]-2,3- dimethyl-phenoxy}-propyl)- acetamide | 542 |
| 685 | 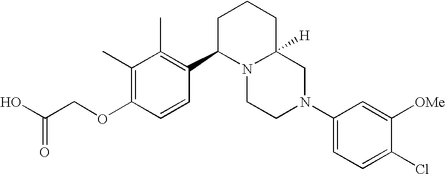 | {4-[(6R,9aS)-2-(4-chloro-3- methoxyphenyl)octahydro-2H- pyrido[1,2-a]pyrazin-6-yl]-2,3- dimethylphenoxy}acetic acid | 459 |
| 686 | 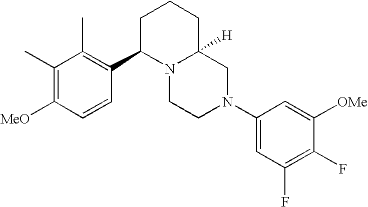 | (6R,9aS)-2-(3,4-difluoro-5- methoxyphenyl)-6-(4-methoxy-2,3- dimethylphenyl)octahydro-2H- pyrido[1,2-a]pyrazine | 417 |
| 687 | 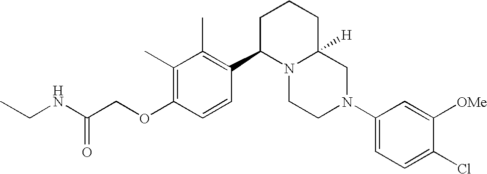 | 2-{4-[(6R,9aS)-2-(4-chloro-3- methoxyphenyl)octahydro-2H- dimethylphenoxy}-N-ethylacetamide | 486 |
| 688 | 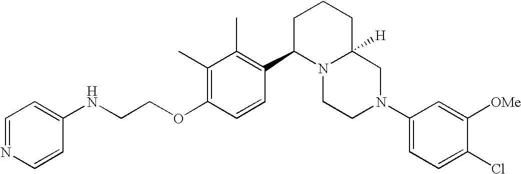 | N-(2-{4-[(6R,9aS)-2-(4-chloro-3- methoxyphenyl)octahydro-2H- pyrido[1,2-a]pyrazin-6-yl]-2,3- dimethylphenoxy}ethyl)pyridin-4- amine | 521 |
| 689 | 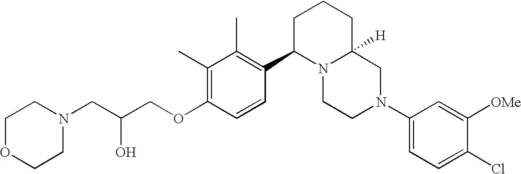 | 1-{4-[(6R,9aS)-2-(4-chloro-3- methoxyphenyl)octahydro-2H- pyrido[1,2-a]pyrazin-6-yl]-2,3- dimethylphenoxy}-3-morpholin-4- ylpropan-2-ol | 544 |
| 690 | 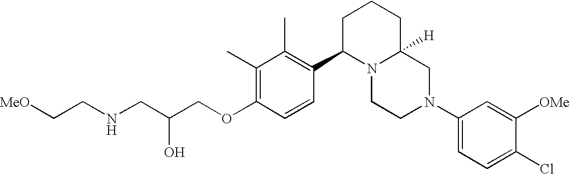 | 1-{4-[(6R,9aS)-2-(4-chloro-3- methoxyphenyl)octahydro-2H- pyrido[1,2-a]pyrazin-6-yl]-2,3- dimethylphenoxy}-3-[(2- methoxyethyl)amino]propan-2-ol | 532 |
| 691 | 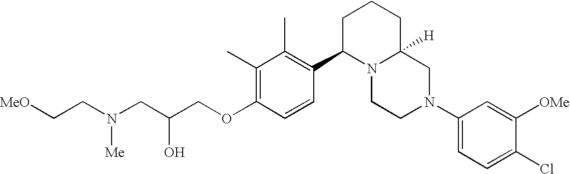 | 1-{4-[(6R,9aS)-2-(4-chloro-3- methoxyphenyl)octahydro-2H- pyrido[1,2-a]pyrazin-6-yl]-2,3- dimethylphenoxy}-3-[(2- methoxyethyl)(methyl)amino]propan- 2-ol | 546 |
| 692 | 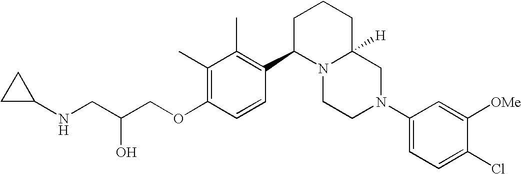 | 1-{4-[(6R,9aS)-2-(4-chloro-3- methoxyphenyl)octahydro-2H- pyrido[1,2-a]pyrazin-6-yl]-2,3- dimethylphenoxy}-3- (cyclopropylamino)propan-2-ol | 514 |
| 693 | 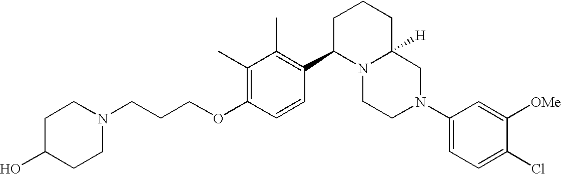 | 1-(3-{4-[(6R,9aS)-2-(4-chloro-3- methoxyphenyl)octahydro-2H- pyrido[1,2-a]pyrazin-6-yl]-2,3- dimethylphenoxy}propyl)piperidin-4- ol | 542 |
| 694 | 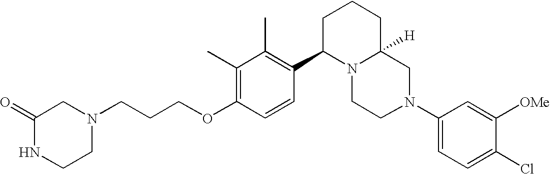 | 4-(3-{4-[(6R,9aS)-2-(4-chloro-3- methoxyphenyl)octahydro-2H- pyrido[1,2-a]pyrazin-6-yl]-2,3- dimethylphenoxy}propyl)piperazin-2- one | 541 |
| 695 | 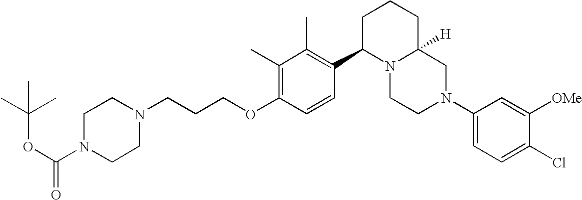 | tert-butyl 4-(3-{4-[(6R,9aS)-2-(4- chloro-3-methoxyphenyl)octahydro- 2H-pyrido[1,2-a]pyrazin-6-yl]-2,3- dimethylphenoxy}propyl)-piperazine- 1-carboxylate | 627 |
| 696 | 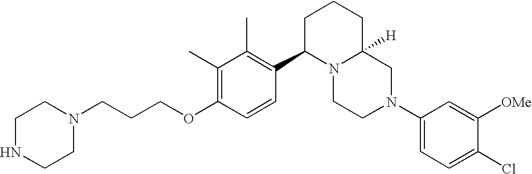 | (6R,9aS)-2-(4-chloro-3- methoxyphenyl)-6-[2,3-dimethyl-4- (3-piperazin-1- ylpropoxy)phenyl]octahydro-2H- pyrido[1,2-a]pyrazine | 527 |
| 697 | 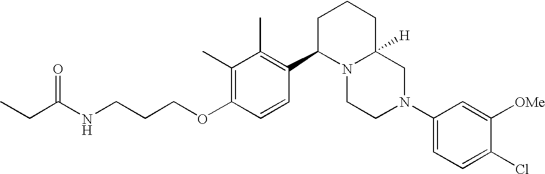 | N-(3-{4-[(6R,9aS)-2-(4-chloro-3- methoxyphenyl)octahydro-2H- pyrido[1,2-a]pyrazin-6-yl]-2,3- dimethylphenoxy}propyl)- propanamide | 514 |
| 698 | 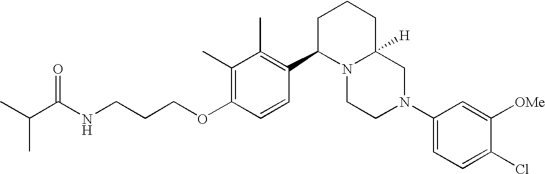 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-2- methylpropanamide | 528 |
| 699 | 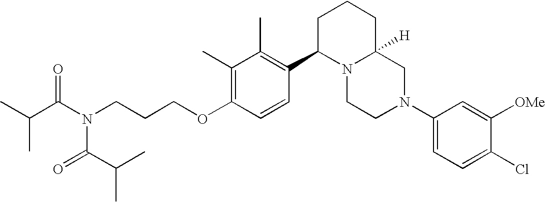 | N-(3-{4-[(6R,9aS)-2-(4-Chloro-3- methoxy-phenyl)-octahydro- pyrido[1,2-a]pyrazin-6-yl]-2,3- dimethyl-phenoxy}-propyl)-N- isobutyryl-isobutyramide | 598 |
| 700 | 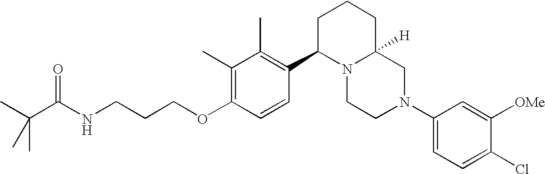 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-2,2- dimethylpropanamide | 542 |
| 701 | 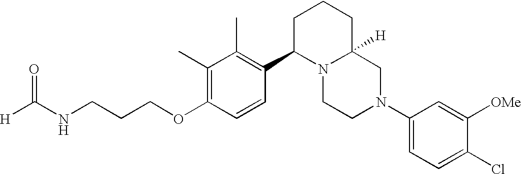 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-formamide | 486 |
| 702 | 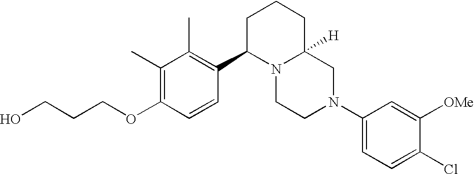 | 3-{4-[2-(4-chloro-3- methoxyphenyl)octahydro-2H- pyrido[1,2-a]pyrazin-6-yl]-2,3- dimethylphenoxy}propan-1-ol | 459 |
| 703 | 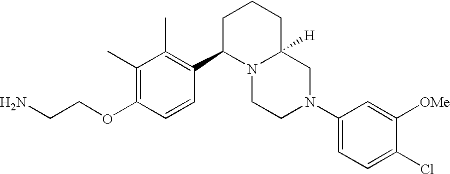 | 2-{4-[(6R,9aS)-2-(4-chloro-3- methoxyphenyl)octahydro-2H- pyrido[1,2-a]pyrazin-6-yl]-2,3- dimethylphenoxy}ethanamine | 444 |
| 704 | 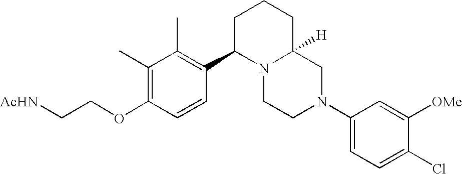 | N-(2-{4-[(6R,9aS)-2-(4-chloro-3- methoxyphenyl)octahydro-2H- pyrido[1,2-a]pyrazin-6-yl]-2,3- dimethylphenoxy}ethyl)acetamide | 486 |
| 705 | 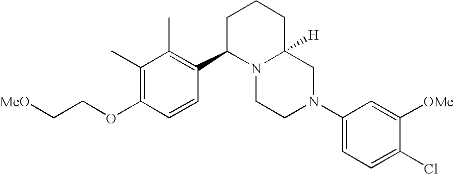 | (6R,9aS)-2-(4-fluoro-3-methoxy- phenyl)-6-[4-(2-methoxyethoxy)-2,3- dimethylphenyl]octahydro-2H- pyrido[1,2-a]pyrazine | 443 |
| 706 | 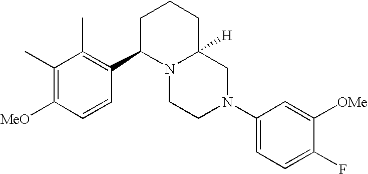 | (6R,9aS)-2-(4-fluoro-3- methoxyphenyl)-6-(4-methoxy-2,3- dimethylphenyl)octahydro-2H- pyrido[1,2-a]pyrazine | 399 |
| 707 | 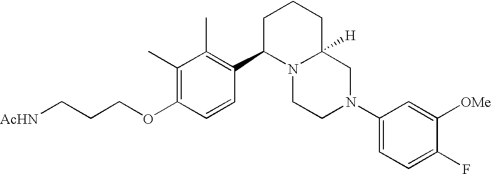 | N-(3-{4-[(6R,9aS)-2-(4-fluoro-3- methoxyphenyl)octahydro-2H- pyrido[1,2-a]pyrazin-6-yl]-2,3- dimethylphenoxy}propyl)acetamide | 484 |
| 708 | 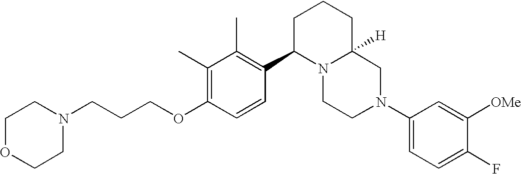 | (6R,9aS)-6-[2,3-dimethyl-4-(3- morpholin-4-ylpropoxy)phenyl]-2-(4- fluoro-3-methoxyphenyl)octa-hydro- 2H-pyrido[1,2-a]pyrazine | 511. 678 |
| 709 | 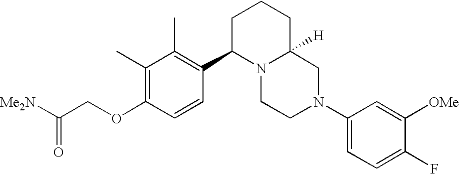 | 2-{4-[(6R,9aS)-2-(4-fluoro-3- methoxyphenyl)octahydro-2H- pyrido[1,2-a]pyrazin-6-yl]-2,3- dimethylphenoxy}-N,N- dimethylacetamide | 470 |
| 710 | 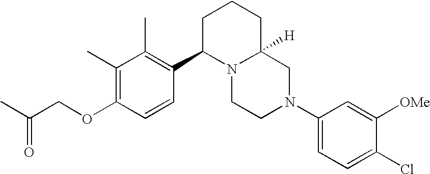 | 1-{4-[(6R,9aS)-2-(4-chloro-3- methoxyphenyl)octahydro-2H- pyrido[1,2-a]pyrazin-6-yl]-2,3- dimethylphenoxy}acetone | 457 |
| 711 | 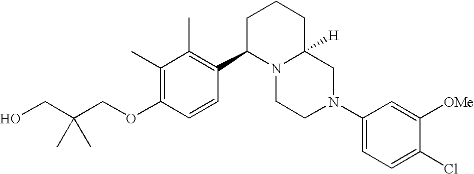 | 3-{4-[(6R,9aS)-2-(4-chloro-3- methoxyphenyl)octahydro-2H- pyrido[1,2-a]pyrazin-6-yl]-2,3- dimethylphenoxy}-2,2- dimethylpropan-1-ol | 487 |
| 712 | 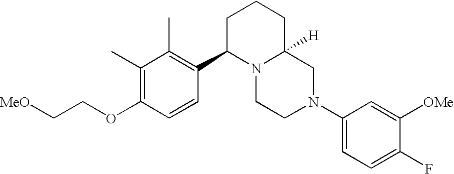 | (6R,9aS)-2-(4-fluoro-3- methoxyphenyl)-6-[4-(2- methoxyethoxy)-2,3- dimethylphenyl]octahydro-2H- pyrido[1,2-a]pyrazine | 443 |
| 713 | 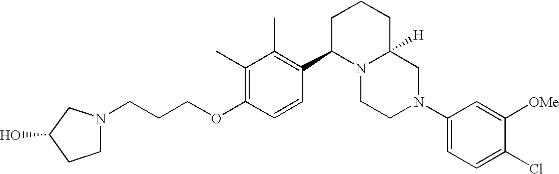 | (3S)-1-(3-{4-[(6R,9aS)-2-(4-chloro- 3-methoxyphenyl)octahydro-2H- pyrido[1,2-a]pyrazin-6-yl]-2,3- dimethylphenoxy}propyl)-pyrrolidin- 3-ol | 528 |
| 714 | 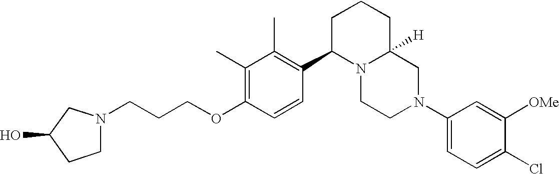 | (3R)-1-(3-{4-[(6R,9aS)-2-(4-chloro- 3-methoxyphenyl)octahydro-2H- pyrido[1,2-a]pyrazin-6-yl]-2,3- dimethylphenoxy}propyl)-pyrrolidin- 3-ol | 528 |
| 715 | 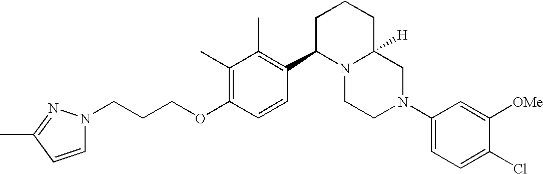 | (6R,9aS)-2-(4-chloro-3- methoxyphenyl)-6-{2,3-dimethyl-4- [3-(3-methyl-1H-pyrazol-1- yl)propoxy]phenyl}octahydro-2H- pyrido[1,2-a]pyrazine | 523 |
| 716 | 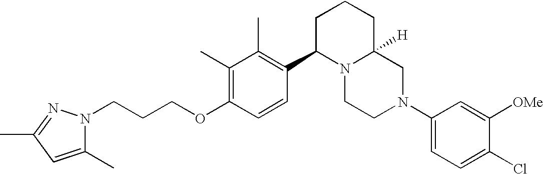 | (6R,9aS)-2-(4-chloro-3- methoxyphenyl)-6-{4-[3-(3,5- dimethyl-1H-pyrazol-1-yl)propoxy]- 2,3-dimethylphenyl}-octahydro-2H- pyrido[1,2-a]pyrazine | 537 |
| 717 | 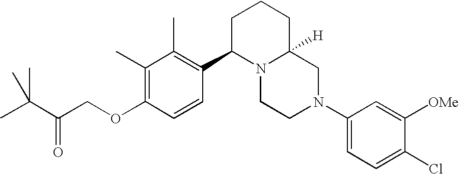 | 1-{4-[(6R,9aS)-2-(4-chloro-3- methoxyphenyl)octahydro-2H- pyrido[1,2-a]pyrazin-6-yl]-2,3- dimethylphenoxy}-3,3- dimethylbutan-2-one | 499 |
| 718 | 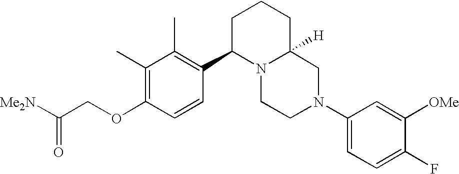 | 2-{4-[(6R,9aS)-2-(4-fluoro-3- methoxyphenyl)octahydro-2H- pyrido[1,2-a]pyrazin-6-yl]-2,3- dimethylphenoxy}-N,N- dimethylacetamide | 470 |
| 719 | 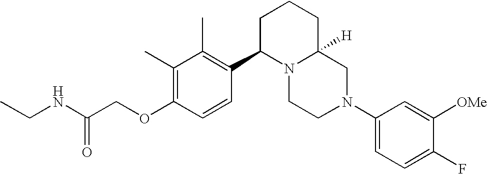 | N-ethyl-2-{4-[(6R,9aS)-2-(4-fluoro- 3-methoxyphenyl)octahydro-2H- pyrido[1,2-a]pyrazin-6-yl]-2,3- dimethylphenoxy}acetamide | 470 |
| 720 | 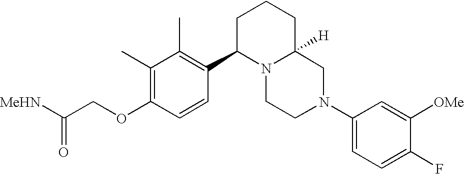 | 2-{4-[(6R,9aS)-2-(4-fluoro-3- methoxyphenyl)octahydro-2H- pyrido[1,2-a]pyrazin-6-yl]-2,3- dimethylphenoxy}-N- methylacetamide | 456 |
| 721 | 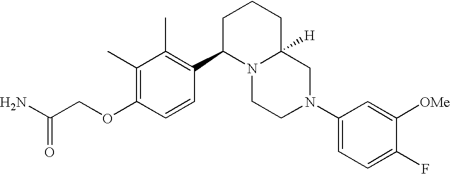 | 2-{4-[(6R,9aS)-2-(4-fluoro-3- methoxyphenyl)octahydro-2H- pyrido[1,2-a]pyrazin-6-yl]-2,3- dimethylphenoxy}acetamide | 442 |
| 722 | 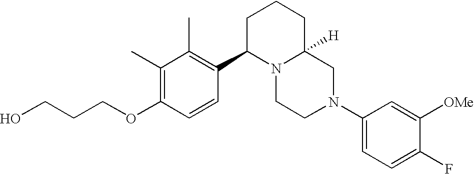 | 3-{4-[(6R,9aS)-2-(4-fluoro-3- methoxyphenyl)octahydro-2H- pyrido[1,2-a]pyrazin-6-yl]-2,3- dimethylphenoxy}propan-1-ol | 443 |
| 723 | 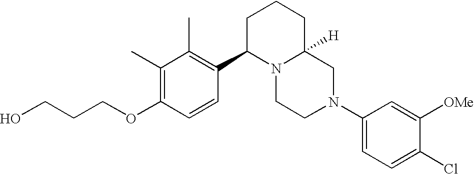 | 3-{4-[(6R,9aS)-2-(4-chloro-3- methoxyphenyl)octahydro-2H- pyrido[1,2-a]pyrazin-6-yl]-2,3- dimethylphenoxy}propan-1-ol | 459 |
| 724 | 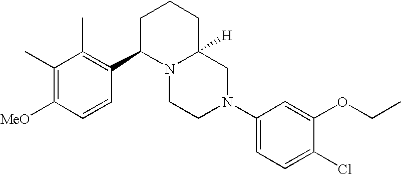 | (6R,9aS)-2-(4-chloro-3- ethoxyphenyl)-6-(4-methoxy-2,3- dimethylphenyl)octahydro-2H- pyrido[1,2-a]pyrazine | 429 |
| 725 | 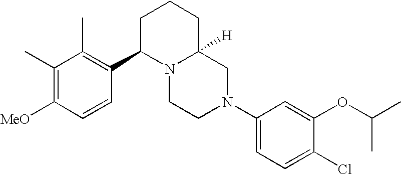 | (6R,9aS)-2-(4-chloro-3- isopropoxyphenyl)-6-(4-methoxy-2,3- dimethylphenyl)octahydro-2H- pyrido[1,2-a]pyrazine | 443 |
| 726 | 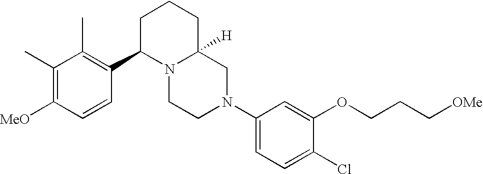 | (6R,9aS)-2-[4-chloro-3-(3- methoxypropoxy)phenyl]-6-(4- methoxy-2,3-dimethylphenyl)octa- hydro-2H-pyrido[1,2-a]pyrazine | 473 |
| 727 | 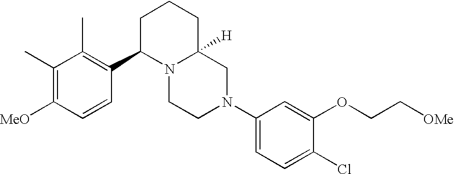 | (6R,9aS)-2-[4-chloro-3-(2- methoxyethoxy)phenyl]-6-(4- methoxy-2,3-dimethylphenyl)octa- hydro-2H-pyrido[1,2-a]pyrazine | 459 |
| 728 | 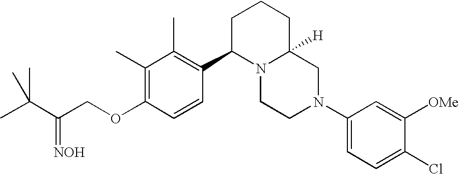 | (2Z)-1-{4-[(6R,9aS)-2-(4-chloro-3- methoxyphenyl)octahydro-2H- pyrido[1,2-a]pyrazin-6-yl]-2,3- dimethylphenoxy}-3,3- dimethylbutan-2-one oxime | 514 |
| 729 | 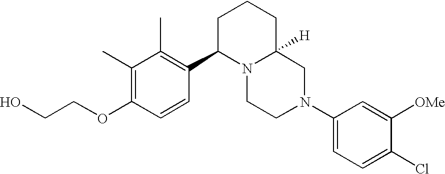 | 2-{4-[(6R,9aS)-2-(4-chloro-3- methoxyphenyl)octahydro-2H- pyrido[1,2-a]pyrazin-6-yl]-2,3- dimethylphenoxy}ethanol | 445 |
| 730 | 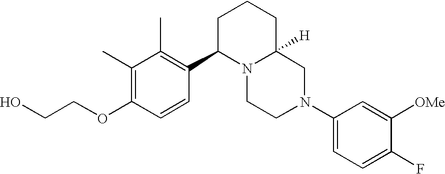 | 2-{4-[(6R,9aS)-2-(4-fluoro-3- methoxyphenyl)octahydro-2H- pyrido[1,2-a]pyrazin-6-yl]-2,3- dimethylphenoxy}ethanol | 429 |
| 731 | 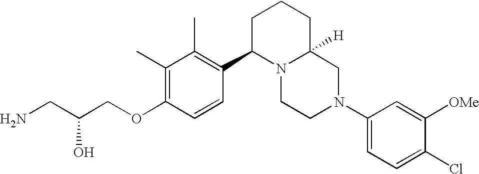 | (2R)-1-amino-3-{4-[(6R,9aS)-2-(4- chloro-3-methoxyphenyl)octahydro- 2H-pyrido[1,2-a]pyrazin-6-yl]-2,3- dimethylphenoxy}propan-2-ol | 474 |
| 732 | 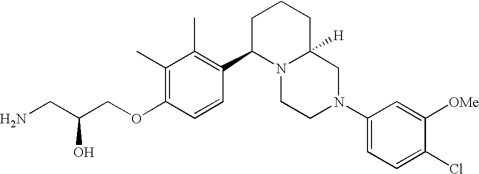 | (25)-1-amino-3-{4-[(6R,9aS)-2-(4- chloro-3-methoxyphenyl)octahydro- 2H-pyrido[1,2-a]pyrazin-6-yl]-2,3- dimethylphenoxy}propan-2-ol | 474 |
| 733 | 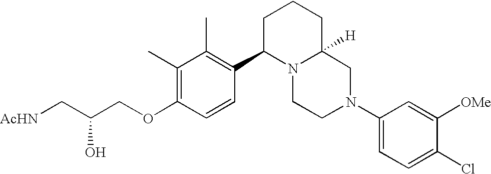 | N-((2R)-3-{4-[(6R,9aS)-2-(4-chloro- methoxyphenyl)octahydro-2H- pyrido[1,2-a]pyrazin-6-yl]-2,3- dimethylphenoxy}-2- hydroxypropyl)acetamide | 516 |
| 734 | 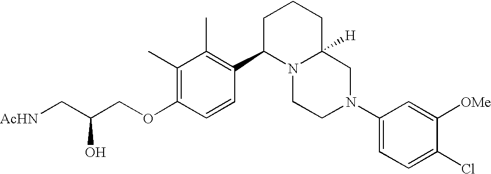 | N-((2S)-3-{4-[(6R,9aS)-2-(4-chloro- 3-methoxyphenyl)octahydro-2H- pyrido[1,2-a]pyrazin-6-yl]-2,3- dimethylphenoxy}-2- hydroxypropyl)acetamide | 516 |
| 735 | 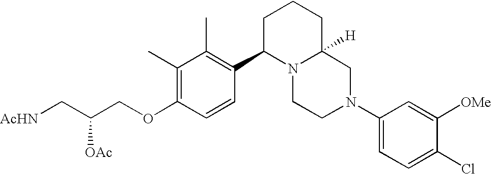 | (1R)-2-(acetylamino)-1-({4- [(6R,9aS)-2-(4-chloro-3- methoxyphenyl)octahydro-2H- pyrido[1,2-a]pyrazin-6-yl]-2,3- dimethylphenoxy}methyl)ethyl acetate | 558 |
| 736 | 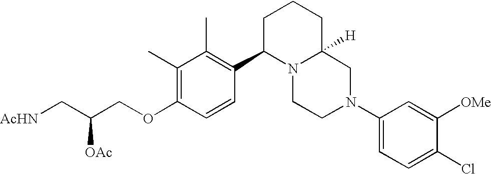 | (1S)-2-(acetylamino)-1-({4- [(6R,9aS)-2-(4-chloro-3- methoxyphenyl)octahydro-2H- pyrido[1,2-a]pyrazin-6-yl]-2,3- dimethylphenoxy}methyl)ethyl acetate | 558 |
| 737 | 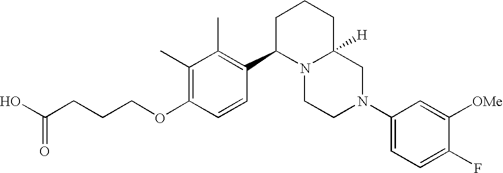 | 4-{4-[(6R,9aS)-2-(4-fluoro-3- methoxyphenyl)octahydro-2H- pyrido[1,2-a]pyrazin-6-yl]-2,3- dimethylphenoxy}butanoic acid | 471 |
| 738 | 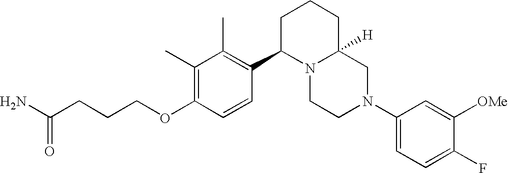 | 4-{4-[(6R,9aS)-2-(4-fluoro-3- methoxyphenyl)octahydro-2H- pyrido[1,2-a]pyrazin-6-yl]-2,3- dimethylphenoxy}butanamide | 470 |
| 739 | 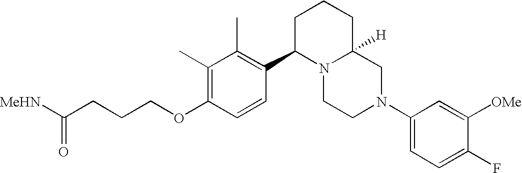 | 4-{4-[(6R,9aS)-2-(4-fluoro-3- methoxyphenyl)octahydro-2H- pyrido[1,2-a]pyrazin-6-yl]-2,3- dimethylphenoxy}-N- methylbutanamide | 484 |
| 740 | 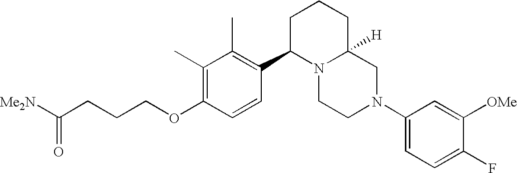 | 4-{4-[(6R,9aS)-2-(4-fluoro-3- methoxyphenyl)octahydro-2H- pyrido[1,2-a]pyrazin-6-yl]-2,3- dimethylphenoxy}-N,N- dimethylbutanamide | 498 |
| 741 | 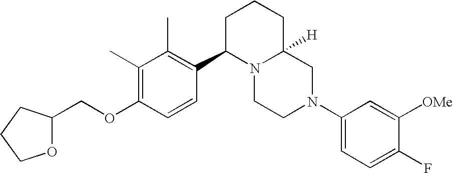 | (6R,9aS)-6-[2,3-dimethyl-4- (tetrahydrofuran-2- ylmethoxy)phenyl]-2-(4-fluoro-3- methoxyphenyl)octahydro-2H- pyrido[1,2-a]pyrazine | 469 |
| 742 | 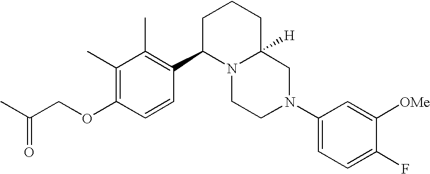 | 1-{4-[(6R,9aS)-2-(4-fluoro-3- methoxyphenyl)octahydro-2H- pyrido[1,2-a]pyrazin-6-yl]-2,3- dimethylphenoxy}acetone | 441 |
| 743 | 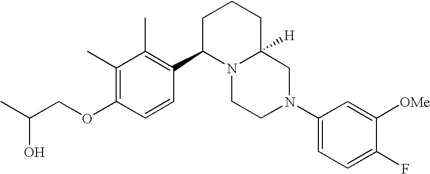 | 1-{4-[(6R,9aS)-2-(4-fluoro-3- methoxyphenyl)octahydro-2H- pyrido[1,2-a]pyrazin-6-yl]-2,3- dimethylphenoxy}propan-2-ol | 443 |
| 744 | 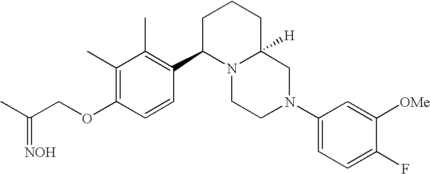 | (2E)-1-{4-[(6R,9aS)-2-(4-fluoro-3- methoxyphenyl)octahydro-2H- pyrido[1,2-a]pyrazin-6-yl]-2,3- dimethylphenoxy}acetone oxime | 456 |
| 745 | 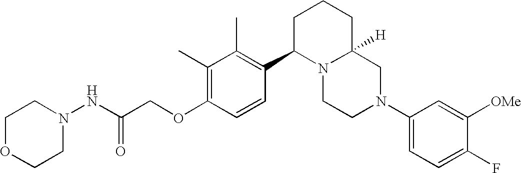 | 2-{4-[(6R,9aS)-2-(4-fluoro-3- methoxyphenyl)octahydro-2H- pyrido[1,2-a]pyrazin-6-yl]-2,3- dimethylphenoxy}-N-morpholin-4- ylacetamide | 527 |
| 746 | 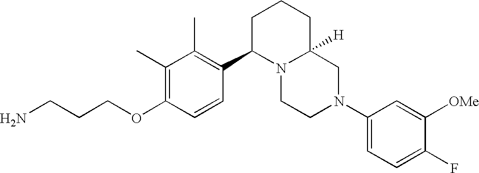 | 3-{4-[(6R,9aS)-2-(4-fluoro-3- methoxyphenyl)octahydro-2H- pyrido[1,2-a]pyrazin-6-yl]-2,3- dimethylphenoxy}propan-1-amine | 442 |
| 747 | 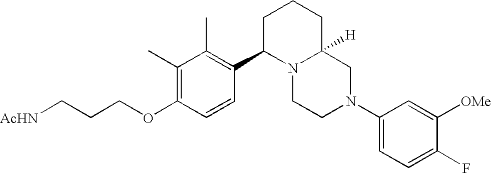 | N-(3-{4-[(6R,9aS)-2-(4-fluoro-3- methoxyphenyl)octahydro-2H- pyrido[1,2-a]pyrazin-6-yl]-2,3- dimethylphenoxy}propyl)acetamide | 484 |
| 748 | 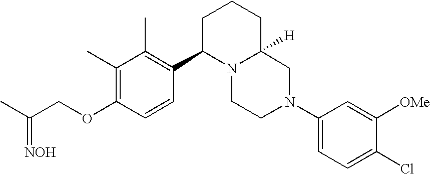 | (2E)-1-{4-{(6R,9aS)-2-(4-chloro-3- methoxyphenyl)octahydro-2H- pyrido[1,2-a]pyrazin-6-yl]-2,3- dimethylphenoxy}acetone oxime | 472 |
| 749 | 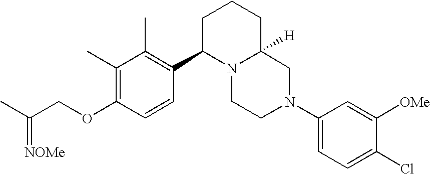 | (2E)-1-{4-[(6R,9aS)-2-(4-chloro-3- methoxyphenyl)octahydro-2H- pyrido[1,2-a]pyrazin-6-yl]-2,3- dimethylphenoxy}acetone O- methyloxime | 486 |
| 750 | 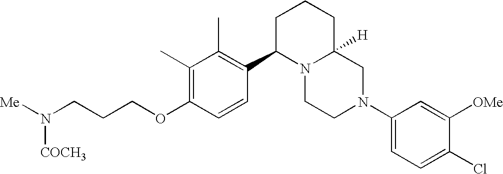 | N-(3-{4-[(6R,9aS)-2-(4-chloro-3- methoxyphenyl)octahydro-2H- pyrido[1,2-a]pyrazin-6-yl]-2,3- dimethylphenoxy}propyl)-N- methylacetamide | 514 |

TABLE IV

| | COMPOUND | NAME | MS |
|---|---|---|---|
| 751 | 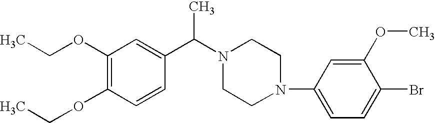 | 1-(4-bromo-3-methoxyphenyl)- 4-[1-(3,4- diethoxyphenyl)ethyl]piperazine | 464 |
| 752 | 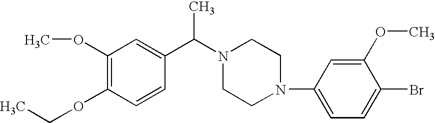 | 1-(4-bromo-3-methoxyphenyl)- 4-[1-(4-ethoxy-3- methoxyphenyl)ethyl]piperazine | 450 |
| 753 | 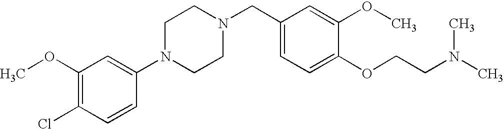 | 2-(4-{[4-(4-chloro-3- methoxyphenyl)piperazin-1- yl]methyl}-2-methoxyphenoxy)- N,N-dimethylethanamine | 434 |
| 754 | 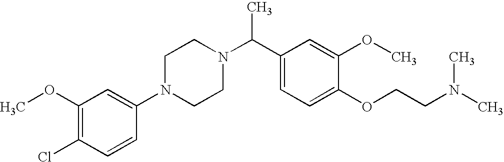 | 2-(4-{1-[4-(4-chloro-3- methoxyphenyl)piperazin-1- yl]ethyl}-2-methoxyphenoxy)-N,N- dimethylethanamine | 448 |
| 755 | 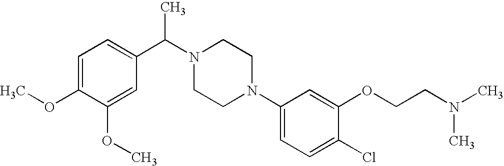 | 2-(2-chloro-5-{4-[1-(3,4- dimethoxyphenyl)ethyl]piperazin- 1-yl}phenoxy)-N,N- dimethylethanamine | 448 |
| 756 | 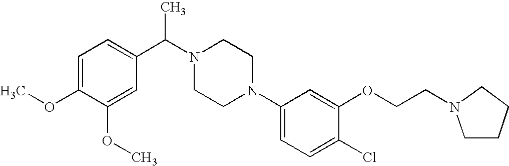 | 1-[4-chloro-3-(2-pyrrolidin-1- ylethoxy)phenyl]-4-[1-(3,4- dimethoxyphenyl)ethyl]piperazine | 474 |
| 757 | 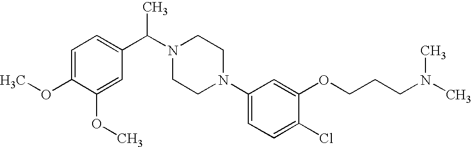 | 3-(2-chloro-5-{4-[1-(3,4- dimethoxyphenyl)ethyl]piperazin- 1-yl}phenoxy)-N,N- dimethylpropan-1-amine | 462 |
| 758 | 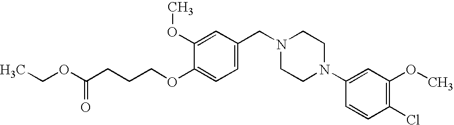 | ethyl 4-(4-{[4-(4-chloro-3- methoxyphenyl)piperazin-1- yl]methyl}-2- methoxyphenoxy)butanoate | 477 |
| 759 | 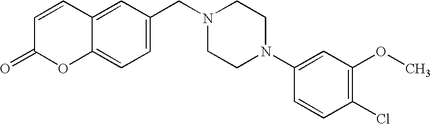 | 6-{[4-(4-chloro-3- methoxyphenyl)piperazin-1- yl]methyl}-2H-chromen-2-one | 385 |
| 760 | 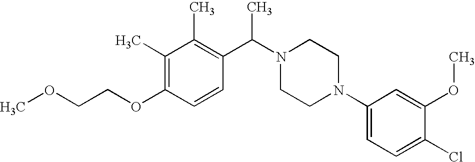 | 1-(4-chloro-3-methoxyphenyl)- 4-{1-[4-(2-methoxyethoxy)-2,3- dimethylphenyi]ethyl}piperazine | 433 |
| 761 | 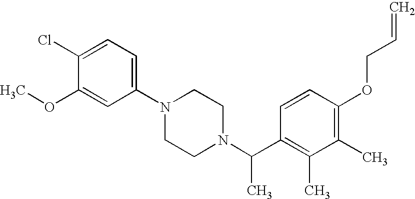 | 1-{1-[4-(allyloxy)-2,3- dimethylphenyl]ethyl}-4-(4- chloro-3-methoxyphenyl)- piperazine | 415 |
| 762 | 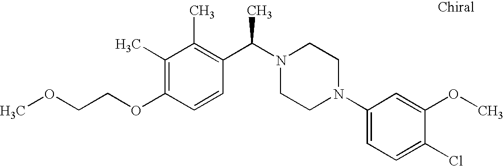 | 1-(4-chloro-3-methoxyphenyl)- 4-{(1R)-1-[4-(2- methoxyethoxy)-2,3- dimethylphenyl]ethyl}piperazine | 433 |
| 763 | 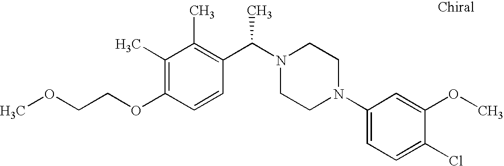 | 1-(4-chloro-3-methoxyphenyl)- 4-{(1S)-1-[4-(2- methoxyethoxy)-2,3- dimethylphenyl]ethyl}piperazine | 433 |
| 764 | 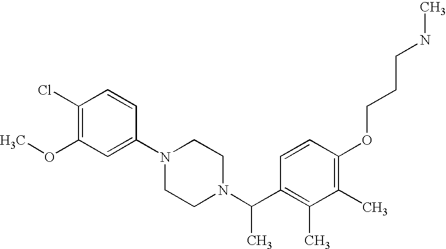 | 3-(4-{1-[4-(4-chloro-3- methoxyphenyl)piperazin-1- yl]ethyl)-2,3-dimethylphenoxy)- N-methylpropan-1-amine | 446 |
| 765 | 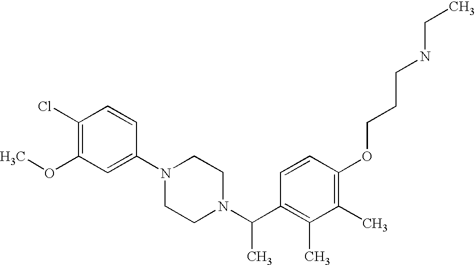 | 3-(4-{1-[4-(4-chloro-3- metboxyphenyl)piperazin-1- yl]ethyl}-2,3-dimethylphenoxy)- N-ethylpropan-1-amine | 460 |
| 766 | 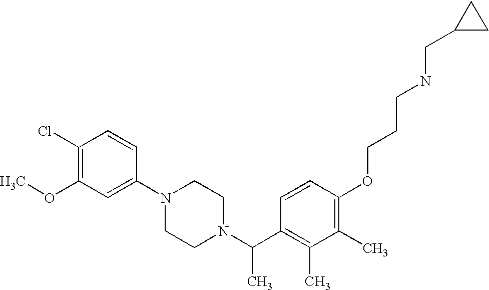 | 3-(4-{1-[4-(4-chloro-3- methoxyphenyl)piperazin-1- yl]ethyl}-2,3-dimethylphenoxy)- N-(cyclopropylmethyl)propan-1- amine | 486 |
| 767 | 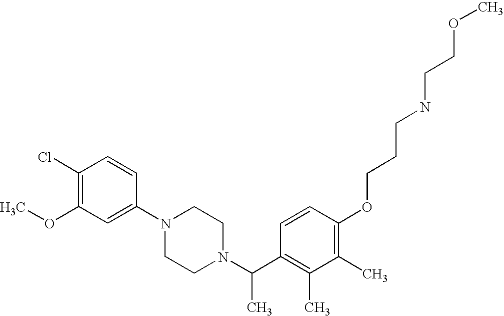 | 3-(4-{1-[4-(4-chloro-3- methoxyphenyl)piperazin-1- yl]ethyl}-2,3-dimethyiphenoxy)- N-(2-methoxyethyl)propan-1- amine | 490 |
| 768 | 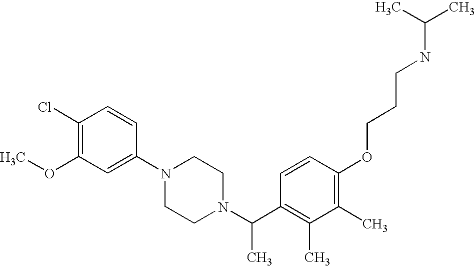 | 3-(4-{1-[4-(4-chloro-3- methoxyphenyl)piperazin-1- yl]ethyl}-2,3-dimethylphenoxy)- N-isopropylpropan-1-amine | 474 |
| 769 | 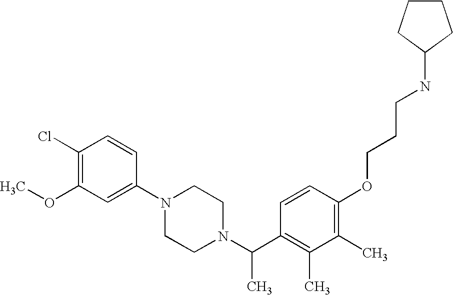 | N-[3-(4-{1-[4-(4-chloro-3- methoxyphenyl)piperazin-1- yl]ethyl}-2,3- dimethylphenoxy)propyl]cyclo pentanamine | 500 |
| 770 | 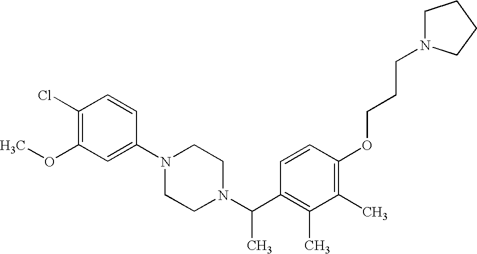 | 1-(4-chloro-3-methoxyphenyl)- 4-{1-[2,3-dimethyl-4-(3- pyrrolidin-1-ylpropoxy)phenyl]- ethyl}piperazine | 486 |
| 771 | 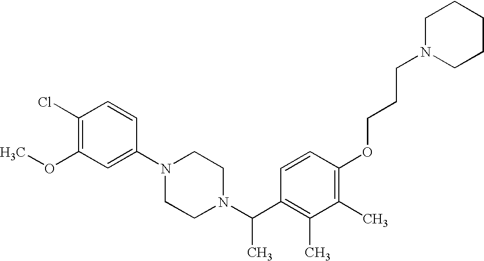 | 1-(4-chloro-3-methoxyphenyl)- 4-{1-[2,3-dimethyl-4-(3- piperidin-1-ylpropoxy)- phenyl]ethyl}piperazine | 500 |
| 772 | 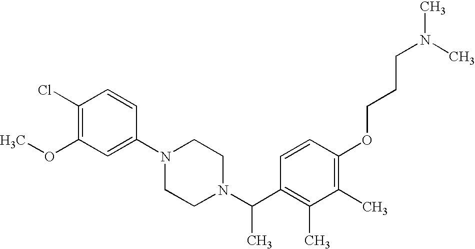 | 3-(4-{1-[4-(4-chloro-3- yl]ethyl}-2,3-dimethylphenoxy)- methoxyphenyl)piperazin-1- N,N-dimethylpropan-1-amine | 460 |
| 773 | 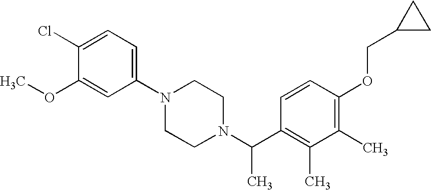 | 1-(4-chloro-3-methoxyphenyl)- 4-{1-[4-(cyclopropylmethoxy)- 2,3-dimethyiphenyl]- ethyl}piperazine | 429 |
| 774 | 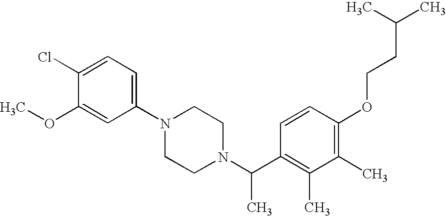 | 1-(4-chloro-3-methoxyphenyl)- 4-{1-[2,3-dimethyl-4-(3- methylbutoxy)phenyl]ethyl}piperazine | 445 |
| 775 | 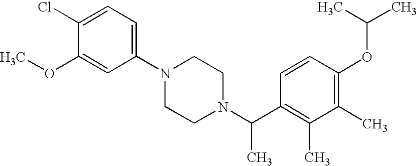 | 1-(4-chloro-3-methoxyphenyl)- 4-[1-(4-isopropoxy-2,3- dimethylphenyl)ethyl]piperazine | 417 |
| 776 | 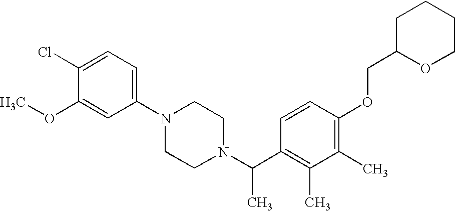 | 1-(4-chloro-3-methoxyphenyl)- (tetrahydro-2H-pyran-2-yl- methoxy)phenyl]ethyl}- piperazine | 473 |
| 777 | 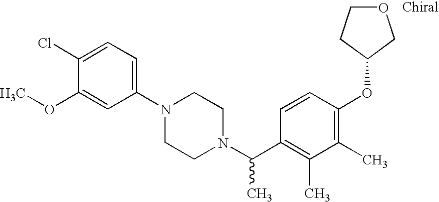 | 1-(4-cbloro-3-methoxyphenyl)- 4-(1-{2,3-dimethyl-4-[(3R)- tetrahydrofuran-3- yloxylphenyl}ethyl)piperazine | 445 |
| 778 | 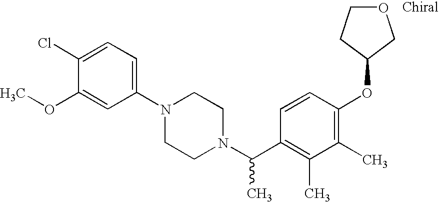 | 1-(4-chloro-3-methoxyphenyl)- 4-(1-{2,3-dimethyl-4-[(3S)- tetrahydrofuran-3-yloxy]phenyl}ethyl) piperazine | 445 |
| 779 | 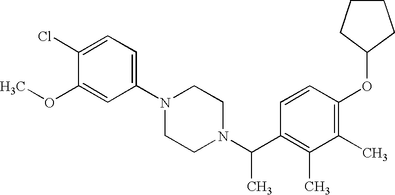 | 1-(4-chloro-3-methoxyphenyl)- 4-{1-[4-(cyclopentyloxy)-2,3- dimethylphenyl]ethyl}piperazine | 443 |
| 780 | 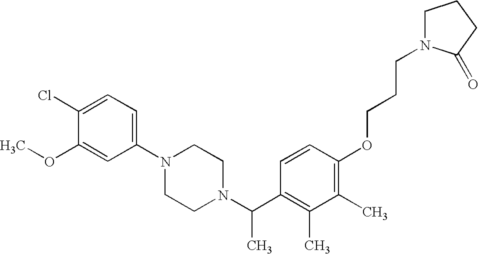 | 1-[3-(4-{1-[4-(4-chloro-3- methoxyphenyl)piperazin-1- yl]ethyl}-2,3- dimethylphenoxy)propyl]pyrrolidin- 2-one | 500 |
| 781 | 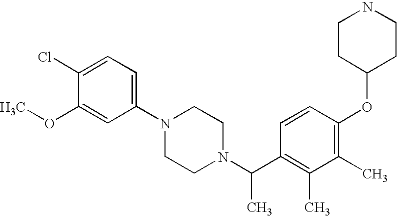 | 1-(4-chloro-3-methoxyphenyl)- 4-{1-[2,3-dimethyl-4-(piperidin- 4-yloxy)phenyllethyl}piperazine | 458 |
| 782 | 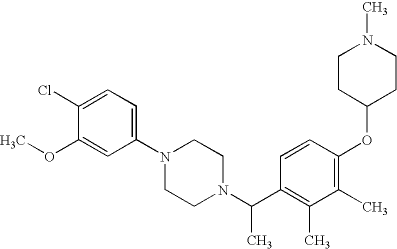 | 1-(4-chloro-3-methoxyphenyl)- 4-(1-{2,3-dimethyl-4-[(1- methylpiperidin-4- yl)oxy]phenyl}ethyl)piperazine | 472 |
| 783 | 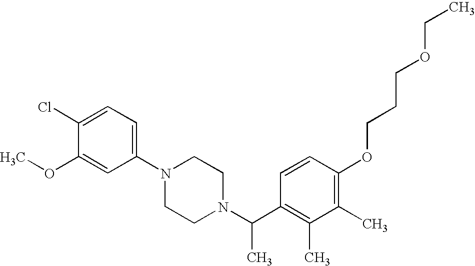 | 1-(4-chloro-3-methoxyphenyl)- 4-{1-[4-(3-ethoxypropoxy)-2,3- dimethylphenyl]ethyl}piperazine | 461 |
| 784 | 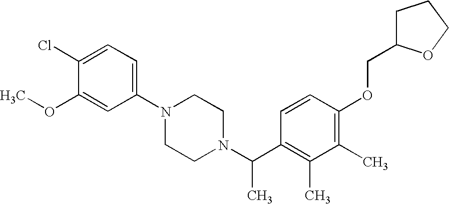 | 1-(4-chloro-3-methoxyphenyl)- 4-{1-[2,3-dimethyl-4- (tetrahydrofuran-2-yl- methoxy)phenyl]ethyl}- piperazine | 459 |
| 785 | 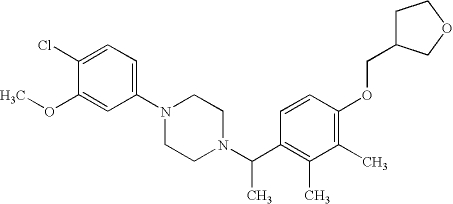 | 1-(4-chloro-3-methoxyphenyl)- 4-{1-[2,3-dimethyl-4- (tetrahydrofuran-3- ylmethoxy)phenyl]ethyl}piperazine | 459 |
| 786 | 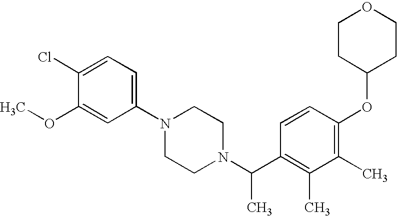 | 1-(4-chloro-3-methoxyphenyl)- 4-{1-[2,3-dimethyl-4- (tetrahydro-2H-pyran-4- yloxy)phenyl]ethyl}piperazine | 459 |
| 787 | 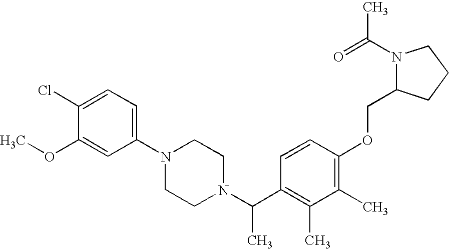 | 1-(1-{4-[(1-acetylpyrrolidin-2- yl)methoxy]-2,3- dimethylphenyl}ethyl)-4-(4- chloro-3-methoxyphenyl)- piperazine | 500 |
| 788 | 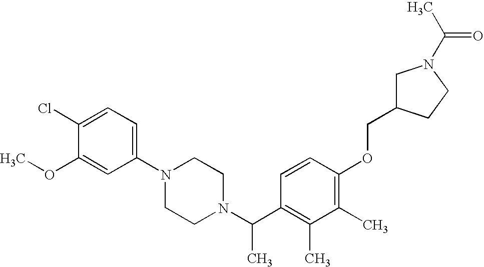 | 1-(1-{4-[(1-acetylpyrrolidin-3- yl)methoxy]-2,3- dimethylphenyl}ethyl)-4-(4- chloro-3-methoxyphenyl)- piperazine | 500 |
| 789 | 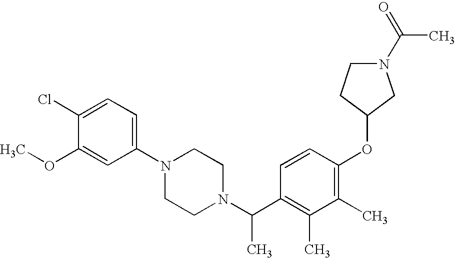 | 1-(1-{4-[(1-acetylpyrrolidin-3- yl)oxy]-2,3- dimethylphenyl}ethyl)-4-(4- chloro-3-methoxyphenyl)- piperazine | 486 |
| 790 | 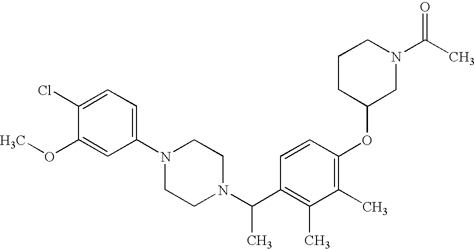 | 1-(1-{4-[(1-acetylpiperidin-3- yl)oxy]-2,3- dimethylphenyl}ethyl)-4-(4- chloro-3- methoxyphenyl)piperazine | 500 |
| 791 | 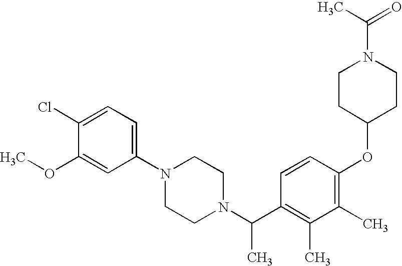 | 1-(1-{4-[(1-acetylpiperidin-4- yl)oxy]-2,3- dimethylphenyl}ethyl)-4-(4- chloro-3- methoxyphenyl)piperazine | 500 |
| 792 | 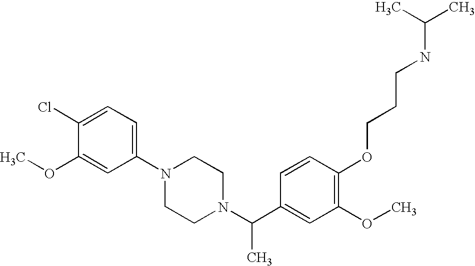 | 3-(4-{1-[4-(4-chloro-3- methoxyphenyl)piperazin-1- yl]ethyl}-2-methoxyphenoxy)- N-isopropylpropan-1-amine | 476 |
| 793 | 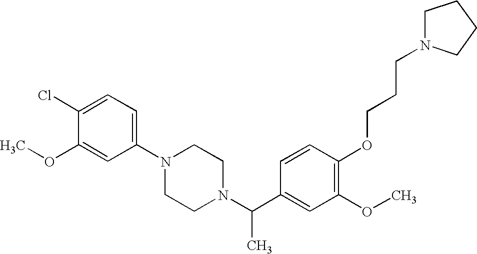 | 1-(4-chloro-3-methoxyphenyl)- 4-{1-[3-methoxy-4-(3- pyrrolidin-1- ylpropoxy)phenyl]ethyl}piperazine | 488 |
| 794 | 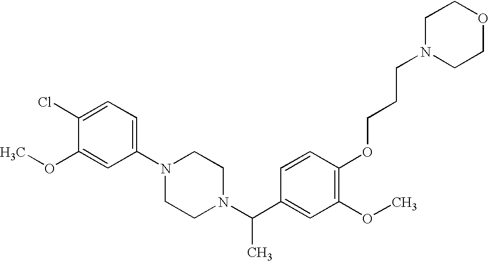 | 4-[3-(4-{1-[4-(4-chloro-3- methoxyphenyl)piperazin-1- yl]ethyl}-2- methoxyphenoxy)propyl]morpholine | 504 |
| 795 | 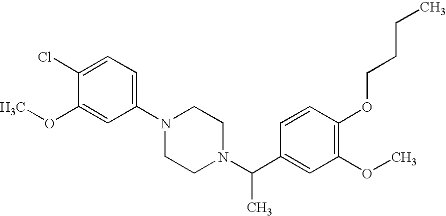 | 1-[1-(4-butoxy-3- methoxyphenyl)ethyl]-4-(4- chloro-3-methoxyphenyl)- piperazine | 433 |
| 796 | 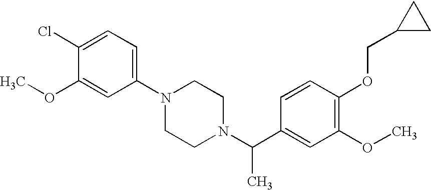 | 1-(4-chloro-3-methoxyphenyl)- 4-{1-[4-(cyclopropylmethoxy)- 3-methoxyphenyl]ethyl}- piperazine | 431 |
| 797 | 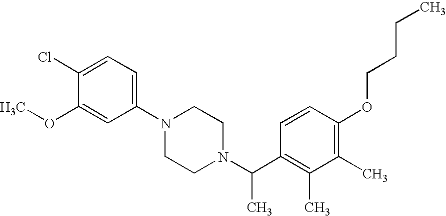 | 1-[1-(4-butoxy-2,3- dimethylphenyl)ethyl]-4-(4- chloro-3-methoxyphenyl)- piperazine | 432 |
| 798 | 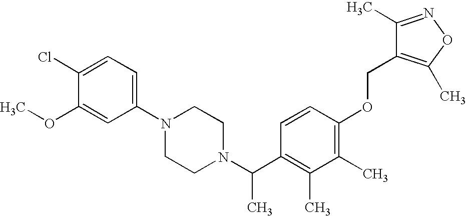 | 1-(4-chloro-3-methoxyphenyl)- 4-(1-{4-[(3,5-dimethylisoxazol- 4-yl)methoxy]-2,3- dimethylphenyl}ethyl)piperazine | 484 |
| 799 | 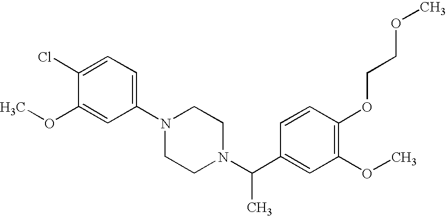 | 1-(4-chloro-3-methoxyphenyl)- 4-{1-[3-methoxy-4-(2- methoxyethoxy)phenyl]ethyl}piperazine | 435 |
| 800 | 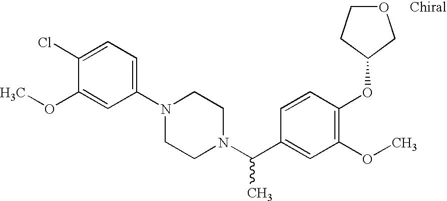 | 1-(4-chloro-3-methoxyphenyl)- 4-(1-{3-methoxy-4-[(3R)- tetrahydrofuran-3- yloxy]phenyl}ethyl)piperazine | 447 |
| 801 | 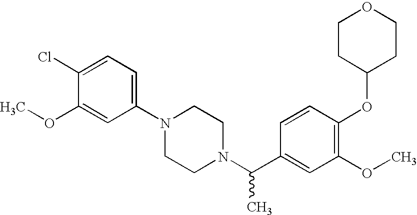 | 1-(4-chloro-3-methoxyphenyl)- 4-{1-[3-methoxy-4-(tetrahydro- 2H-pyran-4- yloxy)phenyl]ethyl}piperazine | 461 |
| 802 | 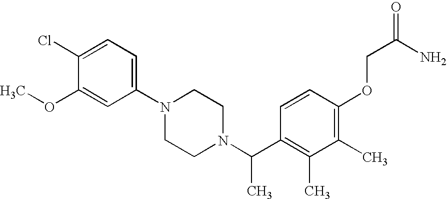 | 2-(4-{1-[4-(4-chloro-3- methoxypheny1)piperazin-1- yl]ethyl}-2,3- dimethylphenoxy)acetamide | 432 |
| 803 | 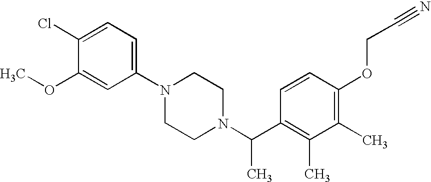 | (4-{1-[4-(4-chloro-3- methoxyphenyl)piperazin-1- yl]ethyl}-2,3- dimethylphenoxy)acetonitrile | 414 |
| 804 | 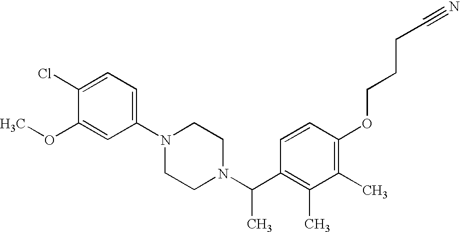 | 4-(4-{1-[4-(4-chloro-3- methoxyphenyl)piperazin-1- yl]ethyl}-2,3- dimethylphenoxy)butanenitrile | 442 |
| 805 | 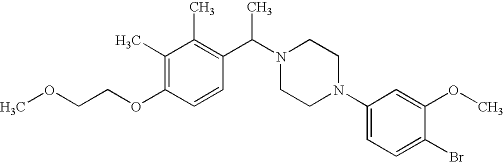 | 1-(4-bromo-3-methoxyphenyl)- 4-{1-[4-(2-methoxyethoxy)-2,3- dimethylphenyl]ethyl}piperazine | 477 |
| 806 | 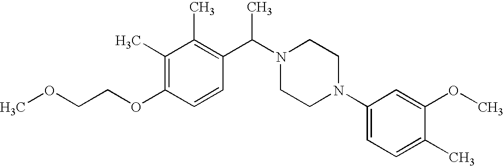 | 1-{1-[4-(2-methoxyethoxy)-2,3- dimethylphenyl]ethyl}-4-(3- methoxy-4- methylphenyl)piperazine | 413 |
| 807 | 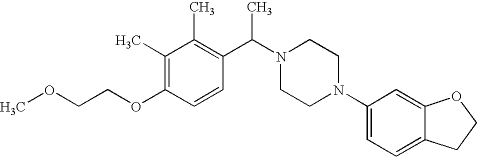 | 1-(2,3-dihydro-1-benzofuran-6- yl)-4-{1-[4-(2-methoxyethoxy)- 2,3-dimethylphenyl]ethyl}- piperazine | 411 |
| 808 | 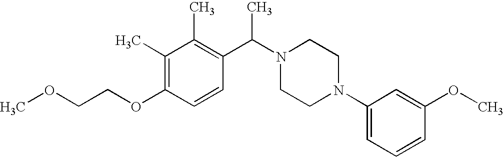 | 1-{1-[4-(2-methoxyethoxy)-2,3- dimethylphenyl]ethyl}-4-(3- methoxyphenyl)piperazine | 399 |
| 809 | 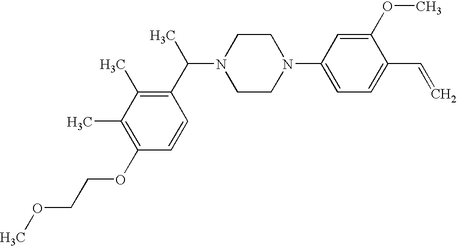 | 1-{1-[4-(2-methoxyethoxy)-2,3- dimethylphenyl]etbyl}-4-(3- methoxy-4-vinylphenyl)- piperazine | 425 |
| 810 | 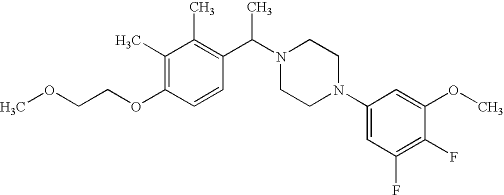 | 1-(3,4-difluoro-5- methoxyphenyl)-4-{1-[4-(2- methoxyethoxy)-2,3- dimethyiphenyl]ethyl}piperazine | 435 |
| 811 | 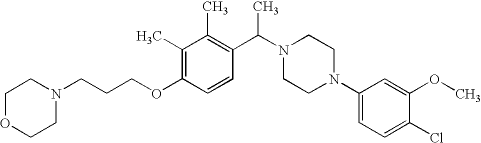 | 4-[3-(4-{1-[4-(4-chloro-3- methoxyphenyl)piperazin-1- yl]ethyl}-2,3-dimethylphenoxy) propyl]morpholine | 502 |
| 812 | 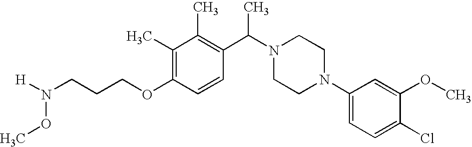 | 3-(4-{1-[4-(4-chloro-3- methoxyphenyl)piperazin-1- yl]ethyl}-2,3-dimethylphenoxy)- N-methoxypropan-1-amine | 462 |
| 813 | 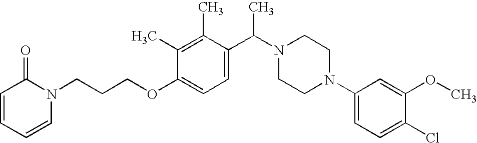 | 1-[3-(4-{1-[4-(4-chloro-3- methoxyphenyl)piperazin-1- yl]ethyl}-2,3- dimethylphenoxy)propyl]pyridin- 2(1H)-one | 510 |
| 814 | 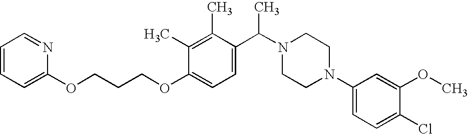 | 1-(4-chloro-3-methoxyphenyl)- 4-(1-{2,3-dimethyl-4-[3- (pyridin-2- yloxy)propoxy]phenyl}ethyl)piperazine | 510 |

TABLE V

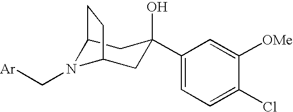

| Cpd. | Ar | NAME | MS |
|---|---|---|---|
| 815 | 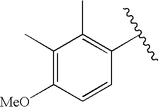 | 3-(4-Chloro-3-methoxyphenyl)-8-(4- methoxy-2,3-dimethylbenzyl)-8- azabicyclo[3.2.1]octan-3-ol | 416 |
| 816 | 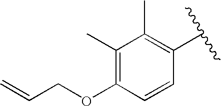 | 8-[4-(Allyloxy)-2,3-dimethylbenzyl]-3-(4- chloro-3-methoxyphenyl)-8- azabicyclo[3.2.1]octan-3-ol | 442 |
| 817 | 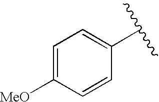 | 3-(4-Chloro-3-methoxyphenyl)-8-(4- methoxybenzyl)-8-azabicyclo[3.2.1]octan- 3-ol | 388 |
| 818 | 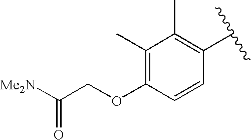 | 2-(4-{[3-(4-Chloro-3-methoxyphenyl)-3- hydroxy-8-azabicyclo[3.2.1]oct-8- yl]methyl}-2,3-dimethylphenoxy)-N,N- dimethylacetamide | 487 |
| 819 | 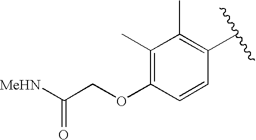 | 2-(4-{[3-(4-Chloro-3-methoxyphenyl)-3- hydroxy-8-azabicyclo[3.2.1]oct-8- yl]methyl}-2,3-dimethylphenoxy)-N- methylacetamide | 473 |
| 820 | 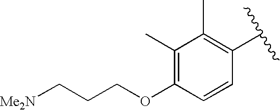 | 3-(4-Chloro-3-methoxyphenyl)-8-{4-[3- (dimethylamino)propoxy]-2,3- dimethylbenzyl}-8-azabicyclo[3.2.1]octan 3-ol | 487 |
| 821 | 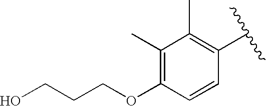 | 3-(4-Chloro-3-methoxyphenyl)-8-[4-(3- hydroxypropoxy)-2,3-dimethylbenzyl]-8- azabicyclo[3.2.1]octan-3-ol | 460 |
| 822 | 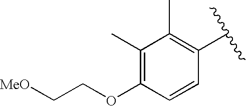 | 3-(4-Chloro-3-methoxyphenyl)-8-[4-(2- methoxyethoxy)-2,3-dimethylbenzyl]-8- azabicyclo[3.2.1]octan-3-ol | 460 |
| 823 | 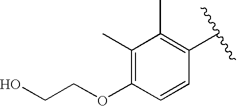 | 3-(4-Chloro-3-methoxyphenyl)-8-[4-(2- hydroxyethoxy)-2,3-dimethylbenzyl]-8- azabicyclo[3.2.1]octan-3-ol | 446 |

TABLE VI

| | COMPOUND | NAME | MS |
|---|---|---|---|
| 824 | 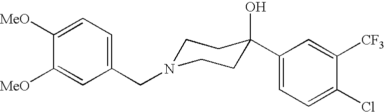 | 4-[4-Chloro-3- (trifluoromethyl)phenyl]-1-(3,4- dimethoxybenzyl)piperidin-4-ol | 430 |
| 825 | 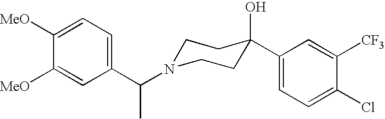 | 4-[4-Chloro-3-(trifluoromethyl)- phenyl]-1-[1-(3,4-dimethoxy- phenyl)ethyl]piperidin-4-ol | 444 |
| 826 | 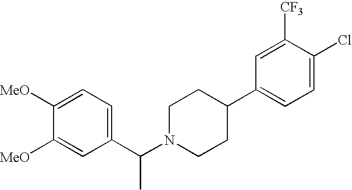 | 4-[4-Chloro-3- (trifluoromethyl)phenyl]-1-[1- (3,4- dimethoxyphenyl)ethyl]piperidine | 428 |
| 827 | 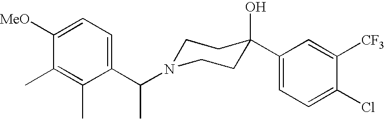 | 4-[4-Chloro-3- (trifluoromethyl)phenyl]-1-[1-(4- methoxy-2,3-dimethylphenyl)- ethyl]piperidin-4-ol | 442 |
| 828 | 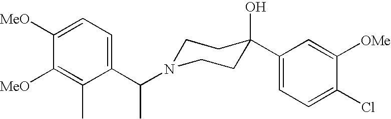 | 4-(4-Chloro-3-methoxyphenyl)-1- [1-(3,4-dimethoxyphenyl)ethyl]- piperidin-4-ol | 406 |
| 829 | 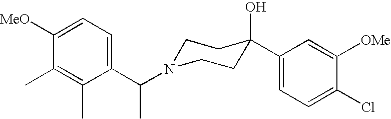 | 4-(4-Chloro-3-methoxyphenyl)-1- [1-(4-methoxy-2,3- dimethylphenyl)ethyl]piperidin-4- ol | 404 |
| 830 | 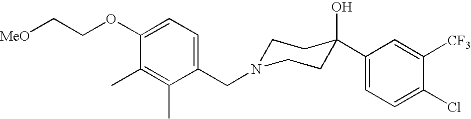 | 4-[4-Chloro-3- (trifluoromethyl)phenyl]-1-[4-(2- methoxyethoxy)-2,3- dimethylbenzyl]piperidin-4-ol | 472 |
| 831 | 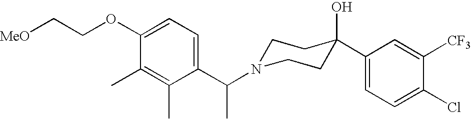 | 4-[4-Chloro-3- (trifluoromethyl)phenyl]-1-(1-[4- (2-methoxyethoxy)-2,3-dimethyl- phenyl]ethyl}piperidin-4-ol | 486 |
| 832 | 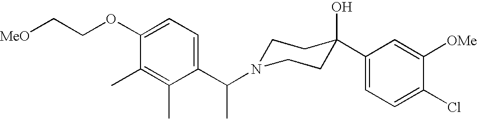 | 4-(4-Chloro-3-methoxyphenyl)-1- {1-[4-(2-methoxyethoxy)-2,3-di- methylphenyl]ethyl}piperidin-4-ol | 448 |
| 833 | 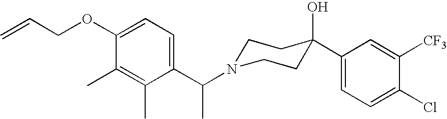 | 1-{1-[4-(Allyloxy)-2,3-dimethyl- phenyl]ethyl}-4-[4-chloro-3-(tri- fluoromethyl)phenyl]piperidin-4- ol | 468 |
| 834 | 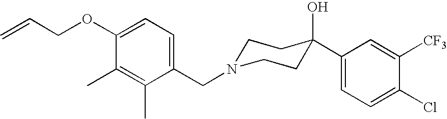 | 1-[4-(Allyloxy)-2,3-dimethyl- benzyl]-4-[4-chloro-3-(trifluoro- methyl)phenyl]piperidin-4-ol | 454 |
| 835 | 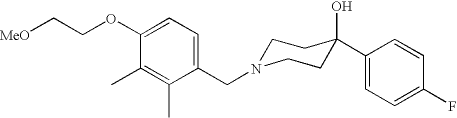 | 4-(4-Fluorophenyl)-1-[4-(2- methoxyethoxy)-2,3- dimethylbenzyl]piperidin-4-ol | 388 |
| 836 | 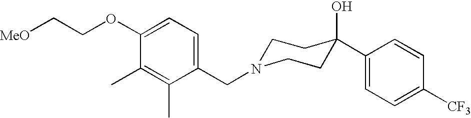 | 1-[4-(2-Methoxyethoxy)-2,3- dimethylbenzyl]-4-[4-(trifluoro- methyl)phenyl]piperidin-4-ol | 438 |
| 837 | 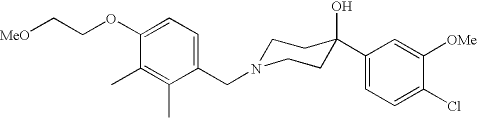 | 4-(4-Chloro-3-methoxy-phenyl)-1- [4-(2-methoxy-ethoxy)-2,3- dimetbyl-benzyl]-piperidin-4-ol | 434 |
| 838 | 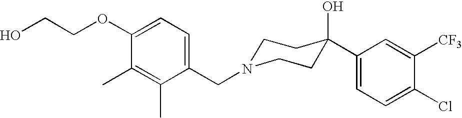 | 4-(4-Chloro-3-trifluoromethyl- phenyl)-1-[4-(2-hydroxy-ethoxy)- 2,3-dimethyl-benzyl]-piperidin-4-ol | 458 |
| 839 | 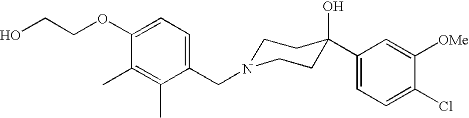 | 4-(4-Chloro-3-methoxy-phenyl)-1- [4-(2-hydroxy-ethoxy)-2,3- dimethyl-benzyl]-piperidin-4-ol | 420 |
| 840 | 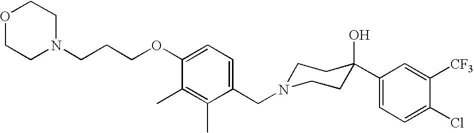 | 4-[4-Chloro-3- (trifluoromethyl)phenyl]-1-[2,3- dimethyl-4-(3-morpholin-4- ylpropoxy)benzyl]piperidin-4-ol | 541 |
| 841 | 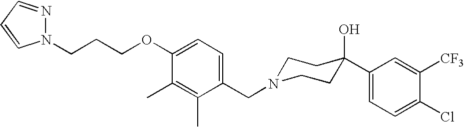 | 4-[4-Chloro-3- (trifluoromethyl)phenyl]-1-{2,3- dimethyl-4-[3-(1H-pyrazol-1- yl)propoxy]benzyl}piperidin-4-ol | 522 |
| 842 | 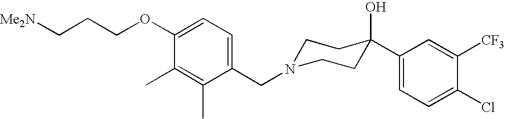 | 4-[4-Chloro-3- (trifluoromethyl)phenyl]-1-{4-[3- (dimethylamino)propoxy]-2,3- dimethylbenzyl}piperidin-4-ol | 499 |
| 843 | 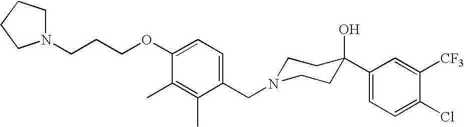 | 4-[4-Chloro-3- (trifluorornethyl)phenyl]-1-[2,3- dimethyl-4-(3-pyrrolidin-1- ylpropoxy)benzyl]piperidin-4-ol | 525 |
| 844 | 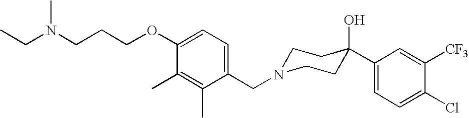 | 4-(4-Chloro-3-trifluoromethyl- phenyl)-1-{4-[3-(ethyl-methyl- amino)-propoxy]-2,3-dimethyl- benzyl}-piperidin-4-ol | 513 |
| 845 | 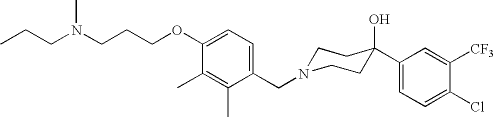 | 4-(4-Chloro-3-trifluoromethyl- phenyl)-1-{2,3-dimethyl-4-[3- (methyl-propyl-amino)-propoxy]- benzyl}-piperidin-4-ol | 527 |
| 846 | 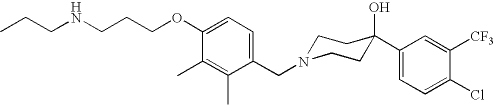 | 4-(4-Chloro-3-trifluoromethyl- phenyl)-1-[2,3-dimethyl-4-(3- propylamino-propoxy)-benzyl]- piperidin-4-ol | 513 |
| 847 | 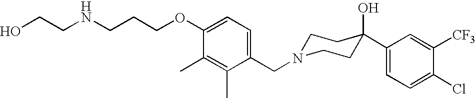 | 4-(4-Chloro-3-trifluoromethyl- phenyl)-1-{4-[3-(2-hydroxy- ethylamino)-propoxy]-2,3- dimethyl-benzyl}-piperidin-4-ol | 515 |
| 848 | 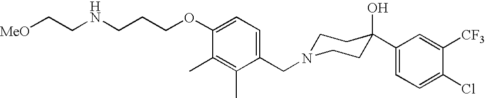 | 4-(4-Chloro-3-trifluoromethyl- phenyl)-1-{4-[3-(2-methoxy- ethylamino)-propoxy]-2,3- dimethyl-benzyl}-piperidin-4-ol | 529 |
| 849 | 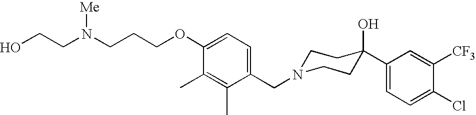 | 4-(4-Chloro-3-trifluoromethyl- phenyl)-1-(4-{3-[(2-hydroxy- ethyl)-methyl-amino]-propoxy}- 2,3-dimethyl-benzyl)-piperidin-4-ol | 529 |
| 850 | 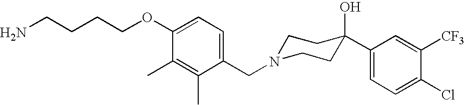 | 1-[4-(4-Amino-butoxy)-2,3- dimethyl-benzyl]-4-(4-chloro-3- trifluoromethyl-phenyl)-piperidin-4-ol | 484 |
| 851 | 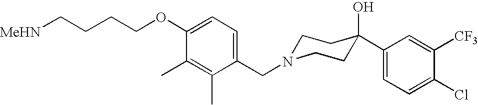 | 4-(4-Chloro-3-trifluoromethyl- phenyl)-1-[2,3 -dimethyl-4-(4- methylamino-butoxy)-benzyl]piperidin-4-ol | 499 |
| 852 | 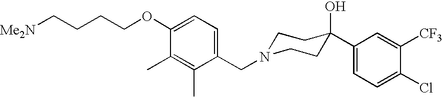 | 4-(4-Chloro-3-trifluoromethyl- phenyl)-1-[4-(4-dimethylamino- butoxy)-2,3-dimethyl-benzyl]- piperidin-4-ol | 513 |
| 853 | 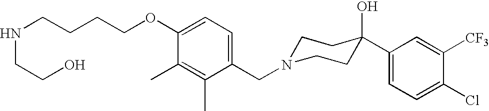 | 4-(4-Chloro-3-trifluoromethyl- phenyl)-1-{4-[4-(2-hydroxy- ethylamino)-butoxy]-2,3- dimethyl-benzyl}-piperidin-4-ol | 529 |
| 854 | 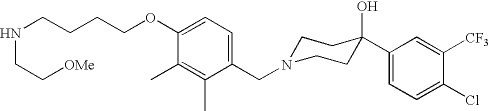 | 4-(4-Chloro-3-trifluoromethyl- phenyl)-1-{4-[4-(2-methoxy- ethylamino)-butoxy]-2,3- dimethyl-benzyl}-piperidin-4-ol | 543 |
| 855 | 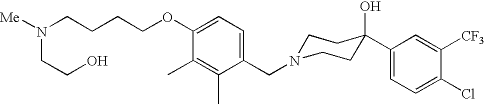 | 4-(4-Chloro-3-trifluoromethyl- phenyl)-1-(4-{4-[(2-hydroxy- ethyl)-methyl-amino]-butoxy}- 2,3-dimethyl-benzyl)-piperidin-4- ol | 543 |
| 856 | 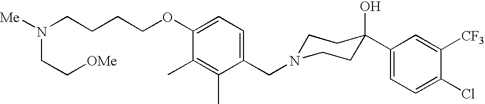 | 4-(4-Chloro-3-trifluoromethyl- phenyl)-1-(4-{4-[(2-methoxy- ethyl)-methyl-amino]-butoxy}- 2,3-dimethyl-benzyl)-piperidin-4- ol | 557 |
| 857 | 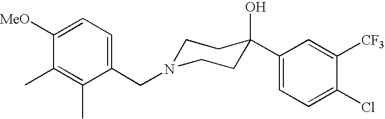 | 4-[4-Chloro-3-(trifluoromethyl)- 4-[4-Chloro-3-(trifluoromethyl)- dimethylbenzyl)piperidin-4-ol | 428 |
| 858 | 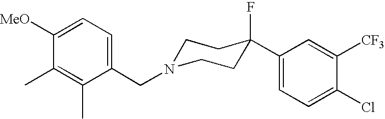 | 4-[4-Chloro-3-(trifluoromethyl)- phenyl]-4-fluoro-1-(4-methoxy- 2,3-dimethylbenzyl)piperidine | 430 |

TABLE VII

| | COMPOUND | NAME | MS |
|---|---|---|---|
| 859 | 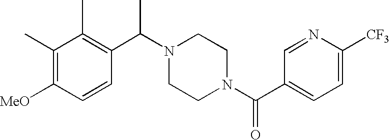 | (4-(1-(4-methoxy-2,3- dimethylphenyl)ethyl)piperazin-1- yl)(6-(trifluoromethyl) pyridin-3- yl)methanone | 422 |
| 860 | 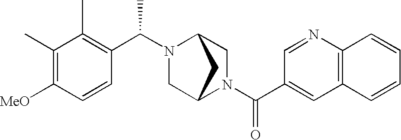 | ((1S,4S)-5-((S)-1-(4-methoxy-2,3- dimethylphenyl)ethyl)-2,5-diaza- bicyclo[2.2.1]heptan-2-yl)(quinolin- 3-yl)methanone | 416 |
| 861 | 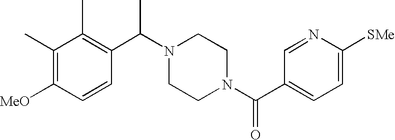 | (4-(1-(4-methoxy-2,3- dimethylphenyl)ethyl)piperazin-1- yl)(6-(methylthio) pyridin-3- yl)methanone | 400 |
| 862 | 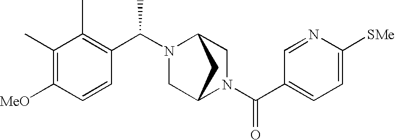 | ((1S,4S)-5-((R)-1-(4-methoxy-2,3- dimetbylphenyl)ethyl)-2,5-diaza- bicyclo[2.2.1]heptan-2-yl)(6- (methylthio)pyridin-3-yl)methanone | 412 |
| 863 | 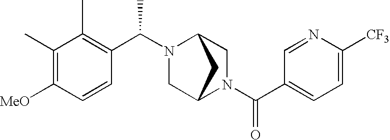 | ((1S,4S)-5-((S)-1-(4-methoxy-2,3- dimethylphenyl)ethyl)-2,5-diaza- bicyclo[2.2.1]heptan-2-yl)(6- (trifluoromethyl)pyridin-3- yl)methanone | 434 |
| 864 | 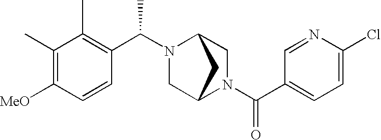 | ((1S,4S)-5-((S)-1-(4-methoxy-2,3- dimethylphenyl)ethyl)-2,5-diaza- bicyclo[2.2.1]heptan-2-yl)(6-chloro- pyridin-3-yl)methanone | 400 |
| 865 | 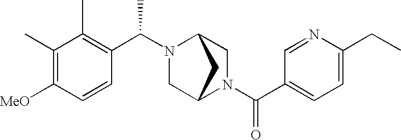 | ((1S,4S)-5-((S)-1-(4-methoxy-2,3- dimethylphenyl)ethyl)-2,5-diaza- bicyclo[2.2.1]heptan-2-yl)(6-ethyl- pyridin-3-yl)methanone | 394 |
| 866 | 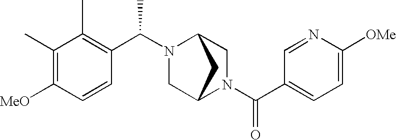 | ((1S,4S)-5-((S)-1-(4-methoxy-2,3- dimethylphenyl)ethyl)-2,5-diaza- bicyclo[2.2.1]heptan-2-yl)(6- methoxy-pyridin-3-yl)methanone | 396 |
| 867 | 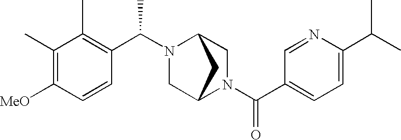 | ((1S,4S)-5-((S)-1-(4-methoxy-2,3- dimethylphenyl)ethyl)-2,5-diaza- bicyclo[2.2.1]heptan-2-yl)(6- isopropyl-pyridin-3-yl)methanone | 408 |
| 868 | 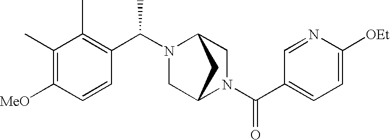 | ((1S,4S)-5-((S)-1-(4-methoxy-2,3- dimethylphenyl)ethyl)-2,5-diaza- bicyclo[2.2.1]heptan-2-yl)(6-ethoxy- pyridin-3-yl)methanone | 410 |
| 869 | 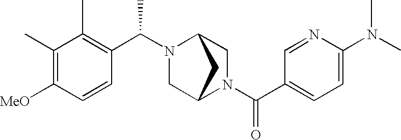 | (6-Dimethylamino-pyridin-3-yl)- {(1S,4S)-5-[(S)-1-(4-methoxy-2,3- dimethyl-phenyl)-ethyl]-2,5-diaza- bicyclo[2.2.1]hept-2-yl}-methanone | 409 |
| 870 | 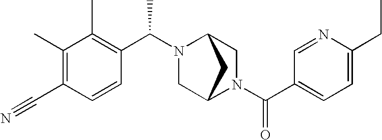 | 4-{(S)-1-[(1S,4S)-5-(6-Ethyl- pyridine-3-carbonyl)-2,5-diaza- bicyclo[2.2.1]hept-2-yl]-ethyl}-2,3- dimethyl-benzonitrile | 389 |
| 871 | 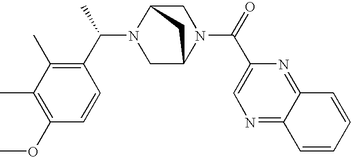 | {(1S,4S)-5-[(S)-1-(4-Methoxy-2,3 - dimethyl-phenyl)-ethyl]-2,5-diaza- bicyclo[2.2.1]hept-2-yl}-quinoxalin- 2-yl-methanone | 417 |

TABLE VIII

| | COMPOUND | NAME | MS |
|---|---|---|---|
| 872 | 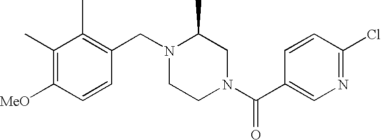 | (6-Chloro-pyridin-3-yl)-[(S)-4- (4-methoxy-2,3-dimethyl- benzyl)-3-methyl-piperazin-1- yl]-methanone | 388 |
| 873 | 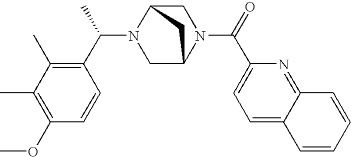 | {(1S,4S)-5-[(S)-1-(4-Methoxy- 2,3-dimethyl-phenyl)-ethyl]- 2,5-diaza-bicyclo[2.2.1]-hept- 2-yl}-quinolin-2-yl-methanone | 416 |
| 874 | 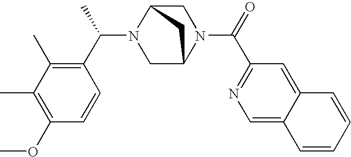 | Isoquinolin-3-yl-{(1S,4S)-5- [(S)-1-(4-methoxy-2,3- dimethylphenyl)-ethyl]-2,5- diaza-bicyclo-[2.2.1]hept-2- yl}-methanone | 416 |
| 875 | 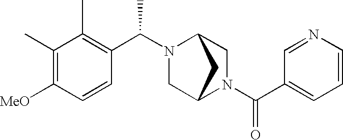 | {(1S,4S)-5-[(S)-1-(4-Methoxy- 2,3-dimethyl-phenyl)-ethyl]- 2,5-diaza-bicyclo[2.2.1]-hept- 2-yl}-pyridin-3-yl-methanone | 366 |
| 876 | 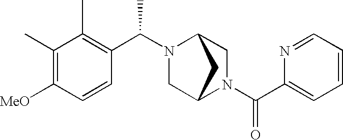 | {(1S,4S)-5-[(S)-1-(4-Methoxy- 2,3-dimethyl-phenyl)-ethyl]- 2,5-diaza-bicyclo-[2.2.1]hept- 2-yl}-pyridin-2-yl-methanone | 366 |
| 877 | 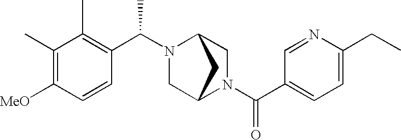 | (6-Ethyl-pyridin-3-yl)- {(1S,4S)-5-[(S)-1-(4-methoxy- 2,3-dimethyl-phenyl)-ethyl]- 2,5-diaza-bicyclo-[2.2.1]hept- 2-yl}-methanone | 394 |
| 878 | 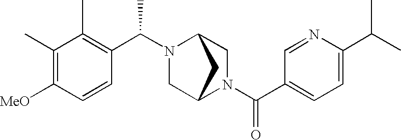 | (6-Isopropyl-pyridin-3-yl)- {(1S,4S)-5-[(S)-1-(4-methoxy- 2,3-dimethylphenyl)-ethyl]-2,5- diaza-bicyclo-[2.2.1]hept-2- yl}-methanone | 408 |
| 879 | 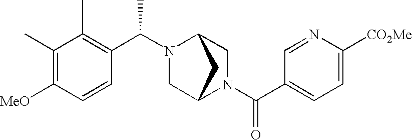 | 6-{(1S,4S)-5-[(S)-1-(4- Methoxy-2,3- dimethylphenyl)-ethyl]2,5- diaza-bicyclo-[2.2.1]heptane-2- carbonyl}-nicotinic acid methyl ester | 422 |
| 880 | 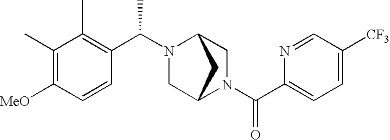 | {(1S,4S)-5-[(S)-1-(4-Methoxy- 2,3-dimethyl-phenyl)-ethyl]- 2,5-diaza-bicyclo-[2.2.1]hept- 2-yl}-(5-trifluoromethyl- pyridin-2-yl)-methanone | 414 |
| 881 | 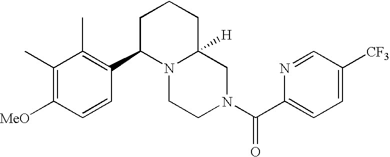 | [(6R,9aS)-6-(4-Methoxy-2,3- dimethylphenyl)-octahydro- pyrido[1,2-a]pyrazin-2-yl]-(5- trifluoromethyl-pyridin-2-yl)- methanone | 448 |
| 882 | 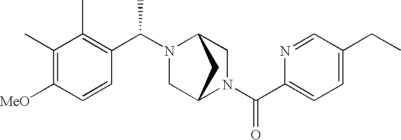 | (5-Ethyl-pyridin-2-yl)- {(1S,4S)-5-[(S)-1-(4-methoxy- 2,3-dimethyl-phenyl)-ethyl]- 2,5-diaza-bicyclo[2.2.1]-hept- 2-yl}-methanone | 394 |
| 883 | 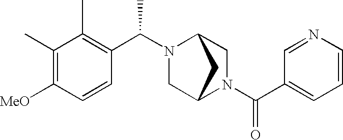 | {(1S,4S)-5-[(S)-1-(4-Methoxy- 2,3-dimethyl-phenyl)-ethyl]- 2,5-diaza-bicyclo[2.2.1]-hept- 2-yl}-pyridin-3-yl-methanone | 366 |
| 884 | 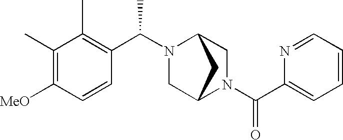 | {(1S,4S)-5-[(S)-1-(4-Methoxy- 2,3-dimethyl-phenyl)-ethyl]- 2,5-diaza-bicyclo-[2.2.1]-hept- 2-yl}-pyridin-2-yl-methanone | 366 |
| 885 | 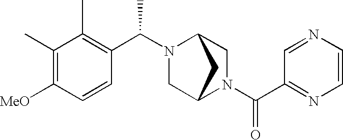 | {(1S,4S)-5-[(S)-1-(4-Methoxy- 2,3-dimethyl-phenyl)-ethyl]- 2,5-diaza-bicyclo-[2.2.1 ]hept- 2-yl}-pyrazin-2-yl-methanone | 367 |
| 886 | 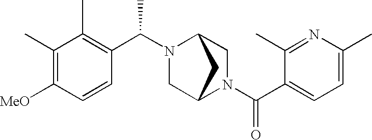 | (2,6-Dimethyl-pyridin-3-yl)- {(1S,4S)-5-[(S)-1-(4-methoxy- 2,3-dimetbylphenyl)-ethyl]-2,5- diazabicyclo[2.2.1]-hept-2-yl}- methanone | 394 |
| 887 | 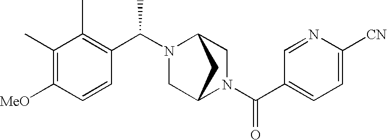 | 5-{(1S,4S)-5-[(S)-1-(4- Methoxy-2,3-dimethyl-phenyl)- ethyl]-2,5-diaza-bicyclo[2.2.1]- heptane-2-carbonyl}-pyridine- 2-carbonitrile | 391 |
| 888 | 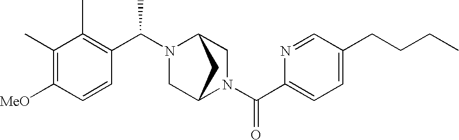 | (5-Butyl-pyridin-2-yl)- {(1S,4S)-5-[(S)-1-(4-methoxy- 2,3-dimethyl-phenyl)-ethyl]- 2,5-diaza-bicyclo[2.2.1]-hept- 2-yl}-methanone | 422 |
| 889 | 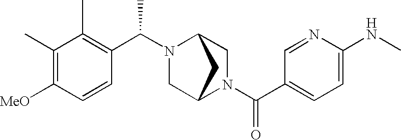 | {(1S,4S)-5-[(S)-1-(4-Methoxy- 2,3-dimethyl-phenyl)-ethyl]- 2,5-diaza-bicyclo[2.2.1]-hept- 2-yl}-(6-methylamino-pyridin- 3-yl)-methanone | 395 |
| 890 | 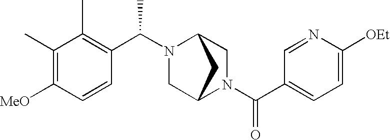 | (6-Ethoxy-pyridin-3-yl)- {(1S,4S)-5-[(S)-1-(4-methoxy- 2,3-dimethylphenyl)-ethyl]-2,5- diaza-bicyclo[2.2.1]hept-2-yl}-methanone | 410 |
| 891 | 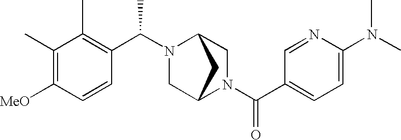 | (6-Dimethylamino-pyridin-3- yl)-{(1S,4S)-5-[(S)-1-(4- methoxy-2,3-dimethyl-phenyl)- ethyl]-2,5-diaza- bicyclo[2.2.1]hept-2-yl}- methanone | 409 |
| 892 | 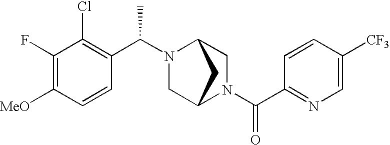 | {(1S,4S)-5-[(S)-1-(2-Chloro-3- fluoro-4-methoxy-phenyl)- bicylo[2.2.1]hept-2-yl}-(5- trifluoromethyl-pyridin-2-yl)- methanone | 458 |
| 893 | 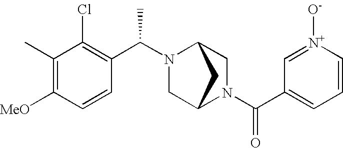 | {(1S,4S)-5-[(S)-1-(2-Chloro-4- methoxy-3-methyl-phenyl)- ethyl]-2,5-diaza-bicyclo- [2.2.1]hept-2-yl}-(1-oxy- pyridin-3-yl)-methanone | 402 |
| 894 | 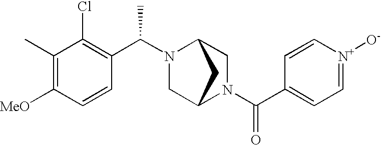 | {(1S,4S)-5-[(S)-1-(2-Chloro-4- methoxy-3-methyl-phenyl)- ethyl]-2,5-diaza-bicyclo- [2.2.1]hept-2-yl}-(1-oxy- pyridin-4-yl)-methanone | 402 |
| 895 | 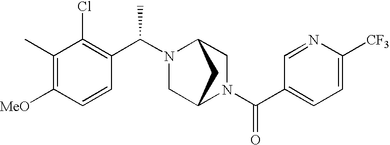 | {(1S,4S)-5-[(S)-1-(2-Chloro-4- methoxy-3-methyl-phenyl)- ethyl]-2,5-diaza-bicyclo- [2.2.1]hept-2-yl}-(6- trifluoromethyl-pyridin-3-yl)- methanone | 454 |
| 896 | 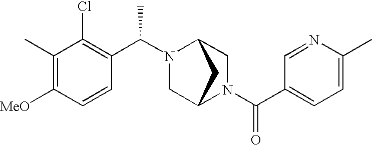 | {(1S,4S)-5-[(S)-1-(2-Chloro-4- methoxy-3-methyl-phenyl)- ethyl]-2,5-diaza-bicyclo- [2.2.1 ]hept-2-yl}-(6-methyl- pyridin-3-yl)-methanone | 400 |
| 897 | 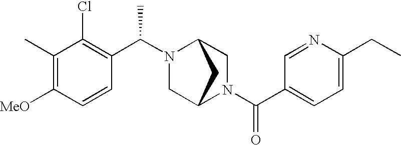 | {(1S,4S)-5-[(S)-1-(2-Chloro-4- methoxy-3-methyl-phenyl)- ethyl]-2,5-diaza-bicyclo- [2.2.1 ]hept-2-yl}-(6-ethyl- pyridin-3-yl)-methanone | 414 |
| 898 | 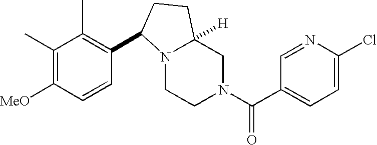 | (6-Chloro-pyridin-3-yl)-[6-(4- methoxy-2,3-dimethyl-phenyl)- hexahydro-pyrrolo[1,2- a]pyrazin-2-yl]-methanone | 400 |
| 899 | 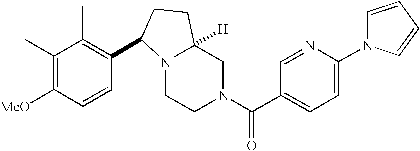 | [6-(4-Methoxy-2,3-dimethyl- phenyl)-hexahydro-pyrrolo[1,2- a]pyrazin-2-yl]-(6-pyrrol-1-yl- pyridin-3- yl)-methanone | 431 |
| 900 | 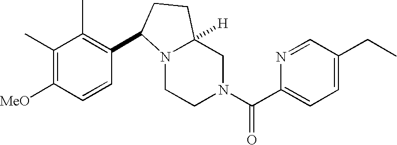 | (5-Ethyl-pyridin-2-yl)-[6-(4- methoxy-2,3-dimethyl-phenyl)- hexahydro-pyrrolo[1,2- a]pyrazin-2-yl]-methanone | 394 |
| 901 | 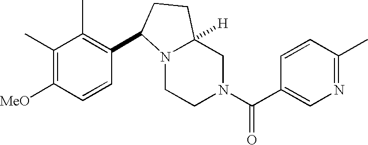 | [6-(4-Methoxy-2,3-dimethyl- phenyl)-hexahydro-pyrrolo[1,2- a]pyrazin-2-yl]-(6-methyl- pyridin-3-yl)-methanone | 380 |
| 902 | 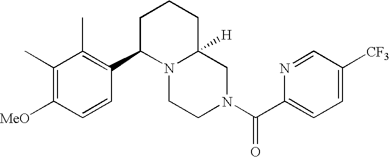 | [(6R,9aS)-6-(4-Methoxy-2,3- dimethyl- phenyl)-octahydro-pyrido[1,2- a]pyrazin-2-yl]-(5- trifluoromethyl-pyridin-2-yl)- methanone | 448 |
| 903 | 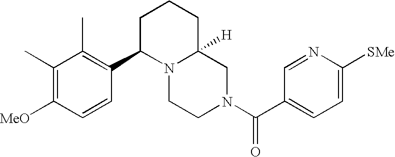 | [(6R,9aS)-6-(4-Methoxy-2,3- dimethyl- phenyl)-octahydro-pyrido[1,2- a]pyrazin-2-yl]-(6- methylsulfanyl-pyridin-3-yl)- methanone | 426 |
| 904 | 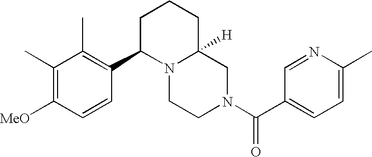 | [(6R,9aS)-6-(4-Methoxy-2,3- dimethyl- phenyl)-octahydro-pyrido[1,2- a]pyrazin-2-yl]-(6-methyl- pyridin-3-yl)-methanone | 394 |
| 905 | 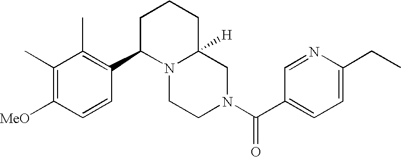 | (6-Ethyl-pyridin-3-yl)- [(6R,9aS)-6- (4-methoxy-2,3-dimethyl- phenyl)-octahydro-pyrido[1,2- a]pyrazin-2-yl]-methanone | 408 |
| 906 | 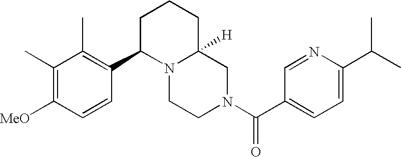 | (6-Isopropyl-pyridin-3-yl)- [(6R,9aS)-6-(4-methoxy-2,3- dimethyl-phenyl)-octahydro- pyrido[1,2-a]pyrazin-2-yl]- methanone | 422 |
| 907 | 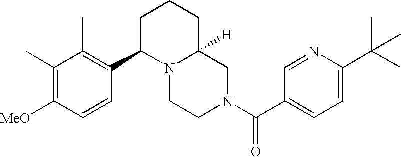 | (6-tert-Butyl-pyridin-3-yl)- [(6R,9aS)-6-(4-methoxy-2,3- dimethyl-phenyl)-octahydro- pyrido[1,2-a]pyrazin-2-yl]- methanone | 436 |
| 908 | 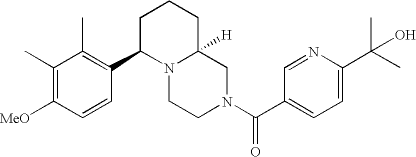 | [6-(1-Hydroxy-1-methyl-ethyl)- pyridin-3-yl]-[(6R,9aS)-6-(4- methoxy-2,3-dimethyl-phenyl)- octahydro-pyrido [1,2- a]pyrazin-2-yl]-methanone | 438 |
| 909 | 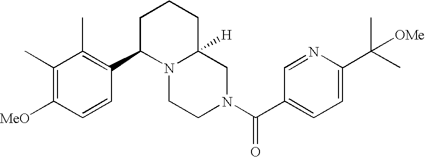 | [(6R,9aS)-6-(4-Methoxy-2,3- dimethyl-phenyl)-octahydro- pyrido[1,2-a]pyrazin-2-yl]-[6- (1-methoxy-1-methyl-ethyl)- pyridin-3-yl]-methanone | 452 |
| 910 | 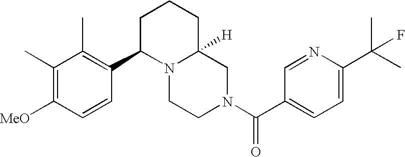 | [6-(1-Fluoro-1-methyl-ethyl)- pyridin-3-yl]-[(6R,9aS)-6-(4- methoxy-2,3-dimethyl-phenyl)- octahydro-pyrido-[1,2- a]pyrazin-2-yl]-methanone | 440 |
| 911 | 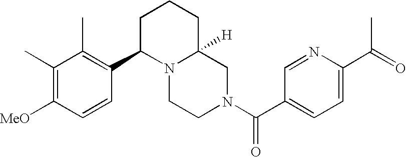 | 1-{5-[(6R,9aS)-6-(4-Methoxy- 2,3-dimethyl-phenyl)- octahydro-pyrido-[1,2- a]pyrazine-2-carbonyl]-pyridin- 2-yl}-ethanone | 422 |
| 912 | 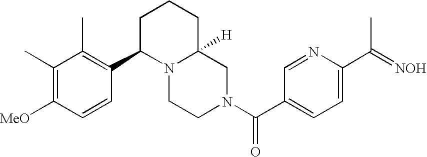 | 1-{5-[(6R,9aS)-6-(4-Methoxy- 2,3-dimethyl-phenyl)- octahydro-pyrido[1,2- a]pyrazine-2-carbonyl]-pyridin- 2-yl}-ethanone oxime | 437 |
| 913 | 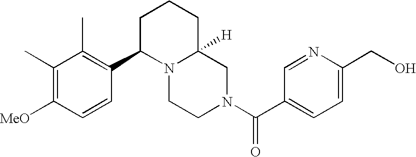 | (6-Hydroxymethyl-pyridin-3- yl)-[(6R,9aS)-6-(4-methoxy- 2,3-dimethyl-phenyl)- octahydro-pyrido[1,2-a]- pyrazin-2-yl]-methanone | 410 |
| 914 | 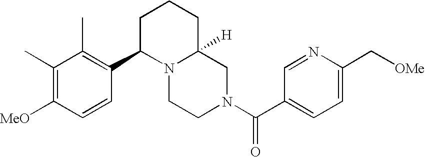 | [(6R,9aS)-6-(4-Methoxy-2,3- dimethyl-phenyl)-octahydro- pyrido[1,2-a]pyrazin-2-yl]-(6- methoxymethyl-pyridin-3-yl)- methanone | 424 |
| 915 | 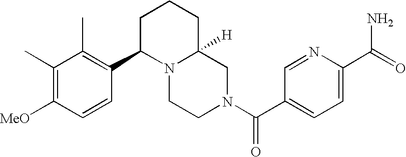 | 5-[(6R,9aS)-6-(4-Methoxy-2,3- dimethyl-phenyl)-octahydro- pyrido[1,2-a)pyrazine-2- carbonyl]-pyridine-2- carboxylic acid amide | 423 |
| 916 | 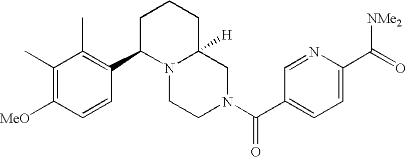 | 5-[(6R,9aS)-6-(4-Methoxy-2,3- dimethyl-phenyl)-octahydro- pyrido[1,2-a]pyrazine-2- carbonyl]-pyridine-2- carboxylic acid dimethylamide | 451 |
| 917 | 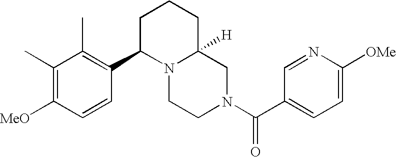 | [(6R,9aS)-6-(4-Methoxy-2,3- dimethyl-phenyl)-octahydro- pyrido[1,2-a]pyrazin-2-yl]-(6- methoxy-pyridin-3-yl)- methanone | 410 |
| 918 | 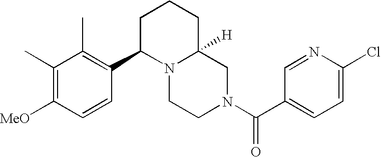 | (6-Chloro-pyridin-3-yl)- [(6R,9aS)-6-(4-methoxy-2,3- dimethyl-phenyl)-octahydro- pyrido[1,2-a]pyrazin-2-yl]- methanone | 414 |
| 919 | 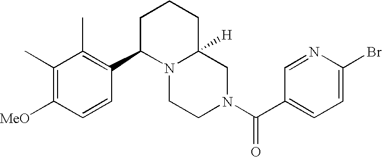 | (6-Bromo-pyridin-3-yl)- [(6R,9aS)-6-(4-methoxy-2,3- dimethyl-phenyl)-octahydro- pyrido[1,2-a]pyrazin-2-yl]- methanone | 458 |
| 920 | 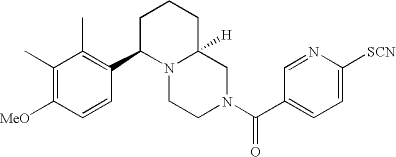 | [6-(4-Methoxy-2,3-dimethyl- phenyl)-octahydro-pyrido[1,2- a]pyrazin-2-yl]-(6-thiocyanato- pyridin-3-yl)-methanone | 437 |
| 921 | 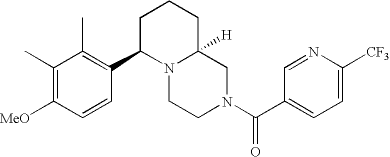 | [(6R,9aS)-6-(4-Methoxy-2,3- dimethylphenyl)-octahydro- pyrido[1,2-a]pyrazin-2-yl]-(6- trifluoromethyl-pyridin-3-yl)- methanone | 448 |
| 922 | 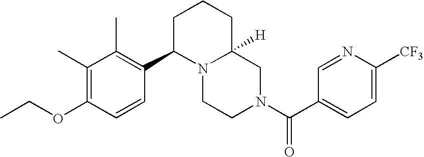 | [(6R,9aS)-6-(4-Ethoxy-2,3- dimethylphenyl)-octahydro- pyrido[1,2-a]pyrazin-2-yl]-(6- trifluoromethyl-pyridin-3-yl)- methanone | 462 |
| 923 | 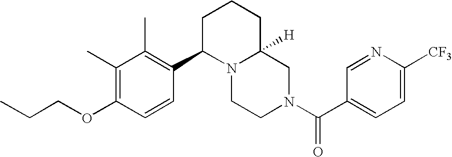 | [(6R,9aS)-6-(2,3-Dimethyl-4- propoxy-phenyl)-octahydro- pyrido[1,2-a]pyrazin-2-yl]-(6- trifluoromethyl-pyridin-3-yl)- methanone | 476 |
| 924 | 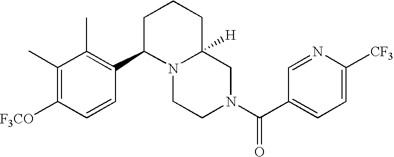 | [(6R,9aS)-6-(2,3 -Dimethyl-4- trifluoromethoxy-phenyl)- octahydro-pyrido[1,2-a]- pyrazin-2-yl]-(6-trifluoro- methyl-pyridin-3-yl)- methanone | 502 |
| 925 | 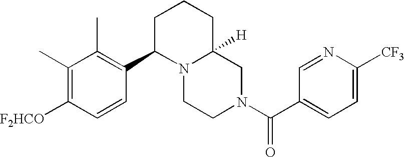 | [(6R,9aS)-6-(4-Difluoro- methoxy-2,3- dimethylphenyl)-octahydro- pyrido[1,2-a]pyrazin-2-yl]-(6- trifluoromethyl-pyridin-3-yl)- methanone | 484 |
| 926 | 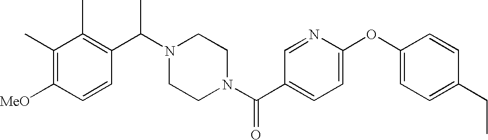 | [6-(4-Ethyl-phenoxy)-pyridin- 3-yl]-{4-[1-(4-methoxy-2,3- dimethyl-phenyl)-ethyl]- piperazin-1-yl}-methanone | 474 |
| 927 | 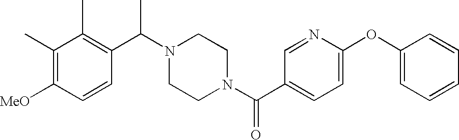 | {4-[1-(4-Methoxy-2,3- dimethylphenyl)-ethyl]piperazin-1-yl}-(6-phenoxy- pyridin-3-yl)-methanone | 446 |
| 928 | 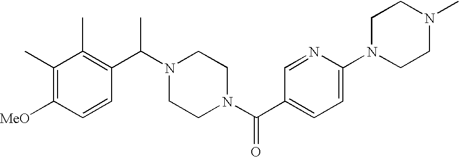 | {4-[1-(4-Methoxy-2,3- dimethyl-phenyl)- ethyl]-piperazin-1-yl}-[6-(4- methyl-piperazin-1-yl)-pyridin- 3-yl]-methanone | 452 |
| 929 | 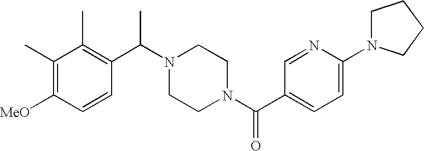 | {4-[l-(4-Methoxy-2,3- dimethyl-phenyl)-ethyl]- piperazin-1-yl}-(6-pyrrolidin-1- yl-pyridin-3-yl)-methanone | 423 |

TABLE LX

| | COMPOUND | NAME | MS |
|---|---|---|---|
| 930 | 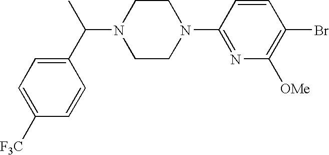 | 1-(5-Bromo-6-methoxy-pyridin-2- yl)-4-[1-(4-trifluoromethyl-phenyl)- ethyl]-piperazine | 444 |
| 931 | 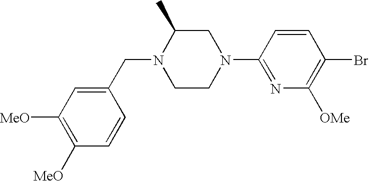 | (S)-4-(S-Bromo-6-methoxy-pyridin- 2-yl)-1-(3,4-dimethoxybenzyl)-2- methyl-piperazine | 436 |
| 932 | 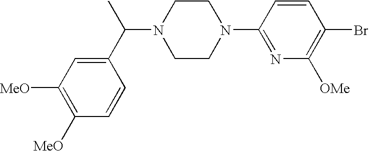 | 1-(5-Bromo-6-methoxy-pyridin-2- yl)-4-[1-(3,4-dimethoxy-phenyl)- ethyl]-piperazine | 436 |
| 933 | 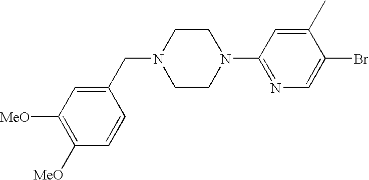 | 1-(S-Bromo-4-methyl-pyridin-2-yl)- 4-(3,4-dimethoxy-benzyl)-piperazine | 406 |
| 934 | 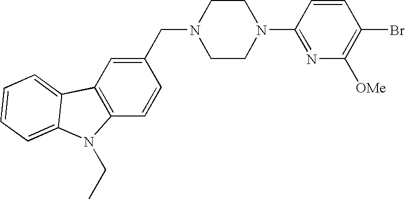 | 3-[4-(5-Bromo-6-methoxy-pyridin-2- yl)-piperazin-1-ylmethyl]-9-ethyl- 9H-carbazole | 479 |
| 935 | 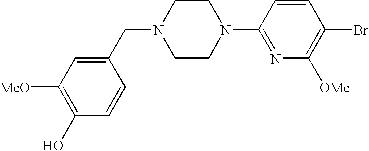 | 4-[4-(5-Bromo-6-methoxy-pyridin-2- yl)-piperazin-1-ylmethyl]-2- methoxy-phenol | 408 |
| 936 | 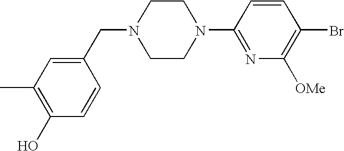 | 1-(5-Bromo-6-methoxy-pyridin-2- yl)-4-(4-methoxy-3-methyl-benzyl)- piperazine | 406 |
| 937 | 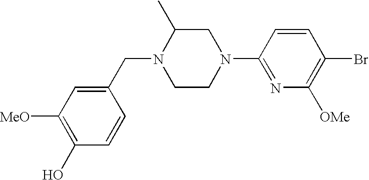 | 4-[4-(5-Bromo-6-methoxy-pyridin-2- yl)-2-methyl-piperazin-1-ylmethyl]- 2-methoxy-phenol | 436 |
| 938 | 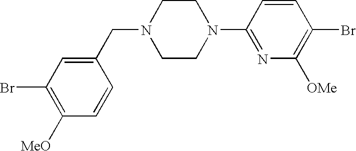 | 1-(3-Bromo-4-methoxy-benzyl)-4-(5- bromo-6-methoxy-pyridin-2-yl)- piperazine | 470 |
| 939 | 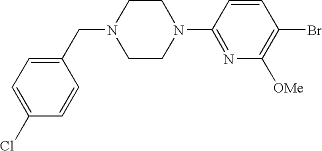 | 1-(5-Bromo-6-methoxy-pyridin-2- yl)-4-(4-chloro-benzyl)-piperazine | 396 |
| 940 | 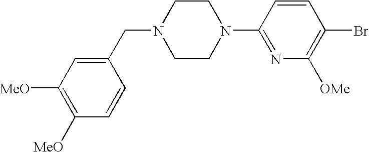 | 1-(5-Bromo-6-methoxy-pyridin-2- yl)-4-(3,4-dimethoxy-benzyl)- piperazine | 422 |
| 941 | 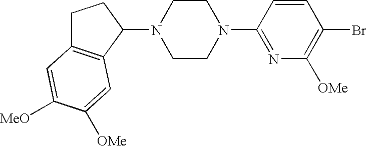 | 1-(5-Bromo-6-methoxy-pyridin-2- yl)-4-(5,6-dimethoxy-indan-1-yl)- piperazine | 448 |
| 942 | 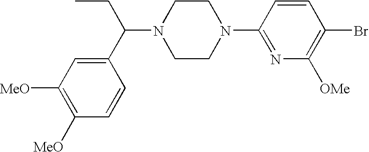 | 1-(5-Bromo-6-methoxy-pyridin-2- yl)-4-[1-(3,4-dimethoxy-phenyl)- propyl]-piperazine | 450 |
| 943 | 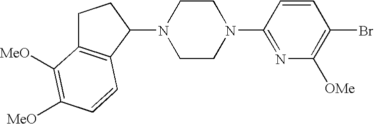 | 1-(5-Bromo-6-methoxy-pyridin-2- yl)-4-(4,5-dimethoxy-indan-1-yl)- piperazine | 448 |

### Example 32

### Purified Rat Striatum Cell Membranes

The MCH1R receptor source is a rat striatum homogenate. The rats are naïve Sprague Dawley or Wistar rats which are not food deprived overnight, and weigh roughly 250±25 grams. The striatum is rapidly/carefully dissected away from the cortex, mid-brain and hippocampus. The striatum is weighed, and homogenized in Prep buffer (50 mM Tris, pH 7.4, 10 mM MgCl₂, 2 mM EGTA: 23 mL per gram of striatum, typically 150 mg of tissue plus 3.5 mL of prep buffer), homogenizing for 30 seconds using a BRINKMAN POLYTRON at setting 5. The crude striatal homogenate is washed 2 times with Prep buffer and sampled for protein analysis between washes. Once the protein concentration has been determined, the final protein pellet is suspended in binding buffer at a protein density of 275 μg/200 μL binding buffer. The protein concentration of the resulting membrane preparation (hereinafter “rat striatal membranes”) is conveniently measured using a Bradford protein assay (Bio-Rad Laboratories, Hercules, Calif.).

### Example 33

### Radioligand Binding Assays

This Example illustrates a standard assay of Melanin Concentrating Hormone receptor binding that may be used to determine the binding affinity of compounds for the MCH receptor. ¹²⁵I-labeled S36057 (New England Nuclear Corp., Boston, Mass.), a stable analogue of MCH, is used as the radioligand.

Purified rat striatal membranes, prepared by the method given above, are resuspended by Dounce homogenization (tight pestle) in binding buffer (50 mM Tris pH. 7.4, 1.0 mM Mg Cl₂, 5 mM KCl, 1 mM CaCl₂, 120 mM NaCl, 1 mM bacitracin, 0.02 mg/mL Aprotinin & 0.1% BSA).

The optimal rat striatal homogenate input has been determined, via a protein linearity experiment, to be 275 μg/data point/250 μL. At 30 pM [¹²⁵I]-S36057, this amount of protein binds 10-15% of the input radioligand. At a [¹²⁵I-S36057 input of 30 pM (roughly ½ to ⅓ Kd) the specific binding signal is routinely 50%. Non specific binding is defined with 1 μM MCH. Displacement binding studies, designed to determine the IC₅₀/Kᵢ of exogenously added compounds, are run at 30 pM [¹²⁵I]-S36057. These displacement studies are routinely run to verify activity in the rat striatum homogenate MCH1R preparation. Upon mixing of all assay components (100 μL tissue, 100 μl assay buffer, 25 μL radiolabel, and 2.5 μL compound if required, 25 μL assay buffer or nonspecific if required), the reaction is mixed and incubated at RT for 2 h in a 96-well deepwell dish. The binding reaction is terminated by rapid filtration over a 1% PEI treated filter on a 96-well Tomtec harvester, followed by washing with 50 mM Tris, pH 7.4, 120 mM NaCl. For saturation binding analysis, rat striatal membranes (275 μg) are added to polypropylene tubes containing 25 pM-0.5 nM [¹²⁵I]S36057. Nonspecific binding is determined in the presence of 10 μM MCH (Tocris Cookson Inc., Ellisville, Mo., USA) and accounts for less than 10% of total binding. For evaluation of guanine nucleotide effects on receptor affinity, GTPγS is added to duplicate tubes at the final concentration of 50 μM.

For competition analysis, membranes (275 μg) are added to polypropylene tubes containing 0.03 nM [¹²⁵I]S36057. Non-radiolabeled displacers are added to separate assays at concentrations ranging from 10⁻¹⁰ M to 10⁻⁵ M to yield a final volume of 0.250 mL. Nonspecific binding is determined in the presence of 10 μM MCH and accounts for less than 30% of total binding. Following a 2-h incubation at room temperature, the reaction is terminated by rapid vacuum filtration. Samples are filtered over presoaked (0.3% non-fat dry milk for 2 h prior to use) GF/C WHATMAN filters and rinsed 2 times with 5 mL cold 50 mM Tris pH 7.4. Remaining bound radioactivity is quantified by gamma counting. Kᵢ and Hill coefficient (“nH”) are determined by fitting the Hill equation to the measured values with the aid of SIGMAPLOT software.

### Example 34

### Purified Recombinant CHO Cell Membranes Expressing Monkey MCH1R

Cynomolgus macaque hypothalamus MCH1R cDNA is prepared and cloned into PCDNA3.1 (INVITROGEN Corp., Carlsbad, Calif.) as described in PCT International Application publication number WO 03/059289, which published on Jul. 24, 2003. The resulting MCH1 expression vector is stably transfected into Chinese hamster ovary (CHO) cells (American Type Culture Collection, Manassas, Va.) via calcium precipitation. The disclosure of WO 03/059289 at page 51-52 directed to the preparation and storage of membrane pellets prepared from CHO cells stably transfected with the MCH1 vector is hereby incorporated by reference.

CHO mMCH1R cell pellets are resuspended in homogenization buffer (10 mM HEPES, 250 mM sucrose, 0.5 μg/mL leupeptin, 2 μg/mL Aprotinin, 200 μM PMSF, and 2.5 mM EDTA, pH 7.4) and homogenized using a BRINKMAN POLYTRON homogenizer (setting 5 for 30 seconds). The homogenate is centrifuged (536×g/10 min/4° C.) to pellet the nuclei. The supernatant containing isolated membranes is decanted to a clean centrifuge tube, centrifuged (48,000×g/30 min, 4° C.) and the resulting pellet resuspended in 30 mL homogenization buffer. This centrifugation and resuspension step is repeated twice. The final pellet is resuspended in ice cold Dulbecco's PBS containing 5 mM EDTA and stored in frozen aliquots at −80° C. until needed. The protein concentration of the resulting membrane preparation (hereinafter “P2 membranes”) is conveniently measured using a Bradford protein assay (Bio-Rad Laboratories, Hercules, Calif.).

### Example 35

### Agonist-Induced GTP Binding

Agonist-stimulated GTP gamma³⁵S binding (“GTP binding”) activity can be used to identify agonist and antagonist compounds and to differentiate neutral antagonist compounds from those that possess inverse agonist activity. This activity can also be used to detect partial agonism mediated by antagonist compounds. A compound being analyzed in this assay is referred to herein as a “test compound.”

Agonist-stimulated GTP binding on purified P2 membranes (prepared as described above) is assessed using MCH as agonist in order to ascertain the level of signal, and EC₅₀ value of MCH as measured by GTP binding.

P2 membranes from the CHO cells are resuspended by Dounce homogenization (tight pestle) in GTP binding assay buffer (50 mM Tris pH 7.4, 120 mM NaCl, 5 mM MgCl2, 2 mM EGTA, 0.1% BSA, 0.1 mM bacitracin, 100 KIU/mL aprotinin, 5 μM GDP, 10 μg/mL saponin) and added to reaction tubes at a concentration of 50 μg protein/reaction tube. After adding increasing doses of the agonist MCH at concentrations ranging from 10⁻¹² M to 10⁻⁶ M, reactions are initiated by the addition of 100 pM GTP gamma³⁵S. In competition experiments, non-radiolabeled test compounds (e.g., compounds provided herein) are added to separate assays at concentrations ranging from 10⁻¹⁰ M to 10⁻⁵ M along with 10 nM MCH to yield a final volume of 0.25 mL.

Neutral antagonists are those test compounds that reduce the MCH stimulated GTP binding activity towards, but not below, baseline (the level of GTP bound by membranes in this assay in the absence of added MCH or other agonist and in the further absence of any test compound).

An antagonist test compound that elevates GTP binding activity above baseline in the absence of added MCH in this GTP binding assay is characterized as having partial agonist activity. Preferred antagonist compounds described herein do not elevate GTP binding activity under such conditions more than 10% above baseline, preferably not more than 5% above baseline, and most preferably not more than 2% above baseline.

Following a 60-min incubation at room temperature, the reactions are terminated by vacuum filtration over GF/C filters (pre-soaked in wash buffer, 0.1% BSA) followed by washing with ice-cold wash buffer (50 mM Tris pH 7.4, 120 mM NaCl). The amount of G-alpha-bound (and thereby membrane-bound) GTP gamma³⁵S is determined by measuring the bound radioactivity, preferably by liquid scintillation spectrometry of the washed filters. Non-specific binding is determined using 10 mM GTP gamma³⁵S and typically represents less than 10% of total binding. Data is expressed as percent above basal (baseline). The results of these GTP binding experiments are analyzed using SIGMAPLOT software and IC₅₀ determined. The IC₅₀ is then used to generate Kᵢ as described by Cheng and Prusoff (1973) Biochem Pharmacol. 22(23):3099-108.

Preferred compounds are MCH1 receptor antagonists that do not possess significant (e.g., greater than 5%) agonist activity in any of the MCH mediated functional assays discussed herein. Specifically, this undesired agonist activity can be evaluated, for example, in the GTP binding assay described above, by measuring small molecule mediated GTP binding in the absence of the agonist, MCH. The preferred extent of MCH1R agonist activity exhibited by compounds of the invention is less than 10%, more preferably less than 5% and most preferably less than 2% of the response elicited by the agonist, MCH.

### Example 36

### Melanin Concentrating Hormone Receptor Binding Assay

This Example illustrates a standard assay of melanin concentrating hormone receptor binding that may be used to determine the binding affinity of compounds for the MCH receptor.

Cynomolgus macaque hypothalamus MCH1R cDNA is prepared and cloned into PCDNA3.1 (INVITROGEN Corp., Carlsbad, Calif.), and HEK293 cells (American Type Culture Collection, Manassas, Va.) are stably transfected with the MCH1 expression vector as described in PCT International Application publication number WO 03/059289, which published on Jul. 24, 2003. The disclosure of WO 03/059289 at page 52 directed to the preparation and storage of the transfected HEK293 cells is hereby incorporated by reference.

At the time of assay, pellets are thawed by addition of wash buffer (25 mM HEPES with 1.0 mM CaCl₂, 5.0 mM MgCl₂, 120 mM NaCl, pH 7.4) and homogenized for 30 seconds using a BRINKMAN POLYTRON, setting 5. Cells are centrifuged for 10 min at 48,000×g. The supernatant is discarded and the pellet is resuspended in fresh wash buffer, and homogenized again. An aliquot of this membrane homogenate is used to determine protein concentration via the Bradford method (BIO-RAD Protein Assay Kit, #500-0001, BIO-RAD, Hercules, Calif.). By this measure, a 1-liter culture of cells typically yields 50-75 mg of total membrane protein. The homogenate is centrifuged as before and resuspended to a protein concentration of 333 μg/mL in binding buffer (Wash buffer+0.1% BSA and 1.0 μM final phosphoramidon) for an assay volume of 50 μg membrane protein/150 μl binding buffer. Phosphoramidon was from SIGMA BIOCHEMICALS, St. Louis, Mo. (cat# R-7385).

Competition binding assays are performed at room temperature in Falcon 96 well round bottom polypropylene plates. Each assay well contains 150 μL of MCH receptor-containing membranes prepared as described above, 50 μL ¹²⁵I-Tyr MCH, 50 μL binding buffer, and 2 μL test compound in DMSO. ¹²⁵I-Tyr MCH (specific activity=2200 Ci/mmol) is purchased from NEN, Boston, Mass. (Cat # NEX 373) and is diluted in binding buffer to provide a final assay concentration of 30 pM.

Non-specific binding is defined as the binding measured in the presence of 1 μM unlabeled MCH. MCH is purchased from BACHEM U.S.A., King of Prussia, Pa. (cat # H-1482). Assay wells used to determine MCH binding contain 150 μL of MCH receptor containing membranes, 50 μL ¹²⁵I-Tyr MCH, 25 μL binding buffer and 25 μL binding buffer.

Assay plates are incubated for 1 h at room temperature. Membranes are harvested onto WALLAC™ glass fiber filters (PERKIN-ELMER, Gaithersburg, Md.) which were pre-soaked with 1.0% PEI (polyethyleneimine) for 2 h prior to use. Filters are allowed to dry overnight, and then counted in a WALLAC 1205 BETA PLATE counter after addition of WALLAC BETA SCINT™ scintillation fluid.

For saturation binding, the concentration of ¹²⁵I-Tyr MCH is varied from 7 to 1,000 pM. Typically, 11 concentration points are collected per saturation binding curve. Equilibrium binding parameters are determined by fitting the allosteric Hill equation to the measured values with the aid of the computer program FitP™ (BIOSOFT, Ferguson, Mo.). For preferred compounds, Kᵢ values are below 1 micromolar, preferably below 500 nanomolar, more preferably below 100 nanomolar.

### Example 37

### Calcium Mobilization Assay

This Example illustrates a representative functional assay for monitoring the response of cells expressing melanin concentrating hormone receptors to melanin concentrating hormone. This assay can also be used to determine if test compounds act as agonists or antagonists of melanin concentrating hormone receptors.

Chinese Hamster Ovary (CHO) cells (American Type Culture Collection; Manassas, Va.) are stably transfected with the MCH expression vector via calcium phosphate precipitation, and are grown to a density of 15,000 cells/well in FALCON™ black-walled, clear-bottomed 96-well plates (#3904, BECTON-DICKINSON, Franklin Lakes, N.J.) in Ham's F12 culture medium (MEDIATECH, Herndon, Va.) supplemented with 10% fetal bovine serum, 25 mM HEPES and 500 μg/mL (active) G418. Prior to running the assay, the culture medium is emptied from the 96 well plates. Fluo-3 calcium sensitive dye (Molecular Probes, Eugene, Oreg.) is added to each well (dye solution: 1 mg FLUO-3 AM, 440 μL DMSO and 440 mL 20% pluronic acid in DMSO, diluted 1:4, 50 μL diluted solution per well). Plates are covered with aluminum foil and incubated at 37° C. for 1-2 h. After the incubation, the dye is emptied from the plates, cells are washed once in 100 μL KRH buffer (0.05 mM KCl, 0.115 M NaCl, 9.6 mM NaH₂PO₄, 0.01 mM MgSO₄, 25 mM HEPES, pH 7.4) to remove excess dye; after washing, 80 μL KRH buffer is added to each well.

Fluorescence response is monitored upon the addition of either human MCH receptor or test compound by a FLIPR™ plate reader (Molecular Devices, Sunnyvale, Calif.) by excitation at 480 nm and emission at 530 nm.

In order to measure the ability of a test compound to antagonize the response of cells expressing MCH receptors to MCH, the EC₅₀ of MCH is first determined. An additional 20 μL of KRH buffer and 1 μL DMSO is added to each well of cells, prepared as described above. 100 μL human MCH in KRH buffer is automatically transferred by the FLIPR instrument to each well. An 8-point concentration response curve, with final MCH concentrations of 1 nM to 3 μM, is used to determine MCH EC₅₀.

Test compounds are dissolved in DMSO, diluted in 20 μL KRH buffer, and added to cells prepared as described above. The 96 well plates containing prepared cells and test compounds are incubated in the dark, at room temperature for 0.5-6 h. It is important that the incubation not continue beyond 6 h. Just prior to determining the fluorescence response, 100 μL human MCH diluted in KRH buffer to 2×EC₅₀ is automatically added by the FLIPR instrument to each well of the 96 well plate for a final sample volume of 200 μL and a final MCH concentration of EC₅₀. The final concentration of test compounds in the assay wells is between 1 nM and 5 μM. Typically, cells exposed to one EC₅₀ of MCH exhibit a fluorescence response of about 10,000 Relative Fluorescence Units. Cells incubated with antagonists of the MCH receptor exhibit a response that is significantly less than that of the control cells to the p≦0.05 level, as measured using a parametric test of statistical significance. Typically, antagonists of the MCH receptor decrease the fluorescence response by about 20%, preferably by about 50%, and most preferably by at least 80% as compared to matched controls. IC₅₀ values for MCHR antagonists are determined using SIGMAPLOT software (SPSS Inc., Chicago, Ill.) and standard techniques. The IC₅₀ is then used to generate Kᵢ as described by Cheng and Prusoff (1973) Biochem Pharmacol. 22(23):3099-108.

The ability of a compound to act as an agonist of the MCH receptor is determined by measuring the fluorescence response of cells expressing MCH receptors, using the methods described above, in the absence of MCH. Compounds that cause cells to exhibit fluorescence above background are MCH receptor agonists (background autofluorescence of the test compound may be assessed using standard methods). Compounds that induce no detectable increase in the basal activity of the MCH receptor have no detectable agonist activity and are preferred.

### Example 38

### MDCK Cytotoxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 μM, 100 μM or 200 μM. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/mL with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% O₂, 5% CO₂ for 2 h with constant shaking. After incubation, 50 μL of mammalian cell lysis solution (from the PACKARD (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 min.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 min. A white PACKARD sticker is attached to the bottom of each plate and samples are dark adapted by wrapping plates in foil and placing in the dark for 10 min. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

### Example 39

### Microsomal In Vitro Half-Life

This Example illustrates the evaluation of compound half-life values (t_{1/2} values) using a representative liver microsomal half-life assay.

Pooled human liver microsomes are obtained from XenoTech LLC (Kansas City, Kans.). Such liver microsomes may also be obtained from In Vitro Technologies (Baltimore, Md.) or Tissue Transformation Technologies (Edison, N.J.). Six test reactions are prepared, each containing 25 μL microsomes, 5 μL of a 100 μM solution of test compound, and 399 μL 0.1 M phosphate buffer (19 mL 0.1 M NaH₂PO₄, 81 mL 0.1 M Na₂HPO₄, adjusted to pH 7.4 with H₃PO₄). A seventh reaction is prepared as a positive control containing 25 μL microsomes, 399 μL 0.1 M phosphate buffer, and 5 μL of a 100 μM solution of a compound with known metabolic properties (e.g., DIAZEPAM or CLOZAPINE). Reactions are preincubated at 39° C. for 10 min.

Cofactor mixture is prepared by diluting 16.2 mg NADP and 45.4 mg glucose-6-phosphate in 4 mL 100 mM MgCl₂. Glucose-6-phosphate dehydrogenase solution is prepared by diluting 214.3 μL glucose-6-phosphate dehydrogenase suspension (Roche Molecular Biochemicals; Indianapolis, Ind.) into 1285.7 μL distilled water. 71 μL of starting reaction mixture (3 mL cofactor mixture; 1.2 mL glucose-6-phosphate dehydrogenase solution) is added to 5 of the 6 test reactions and to the positive control. 71 μL 100 mM MgCl₂ is added to the sixth test reaction, which is used as a negative control. At each time point (0, 1, 3, 5 and 10 min), 75 μL of each reaction mix is pipetted into a well of a 96-well deep-well plate containing 75 μL ice-cold acetonitrile. Samples are vortexed and centrifuged 10 min at 3500 rpm (Sorval T 6000D centrifuge, H1000B rotor). 75 μL of supernatant from each reaction is transferred to a well of a 96-well plate containing 150 μL of a 0.5 μM solution of a compound with a known LC/MS profile (internal standard) per well. LC/MS analysis of each sample is carried out and the amount of unmetabolized test compound is measured as AUC, compound concentration vs. time is plotted, and the t_{1/2} value of the test compound is extrapolated. Preferred compounds provided herein exhibit in vitro t_{1/2} values of greater than 10 min and less than 4 h, preferably between 30 min and 1 h, in human liver microsomes.

From the foregoing it will be appreciated that, although specific embodiments have been described herein for purposes of illustration, various modifications may be made without deviating from the spirit and scope of the invention.

1. (canceled)

2. A compound of the formula: 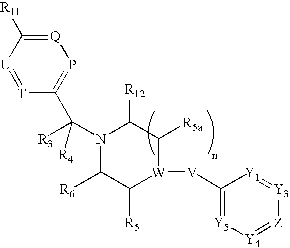
or a pharmaceutically acceptable salt thereof, wherein:
V is absent or —C═O)—;
W is N, CH or C—OH;
Y₁, Y₃, Y₄, and Y₅ are independently CR₁ or nitrogen;
Z is nitrogen or CR₂;
each R₁ is independently:
(i) hydrogen, halogen, hydroxy, nitro, cyano, amino, aminocarbonyl, C₁-C₆alkyl, C₂-C₆alkenyl, C₂-C₆alkynyl, C₁-C₆alkoxy, haloC₁-C₆alkyl, haloC₁-C₆alkoxy, hydroxyC₁-C₆alkyl, (C₁-C₄alkoxy)C₁-C₄alkyl, C₁-C₆alkylthio, aminoC₁-C₆alkyl, mono- or di-(C₁-C₆alkyl)aminoC₀-C₆alkyl, mono- or di-(C₁-C₆alkyl)aminocarbonyl, (C₃-C₇cycloalkyl)C₀-C₆alkyl, or (4- to 7-membered heterocycloalkyl)C₀-C₆alkyl; or
(ii) taken together with R₂ to form a fused 5- or 6-membered carbocycle or heterocycle, each of which is substituted with from 0 to 3 substituents independently chosen from halogen, hydroxy, nitro, cyano, amino, C₁-C₄alkyl, C₁-C₄alkoxy, haloC₁-C₄alkyl, and haloC₁-C₄alkoxy;
R₂ is halogen, hydroxy, nitro, cyano, amino, acetyl, aminocarbonyl, imino, C₁-C₆alkyl, C₂-C₆alkenyl, C₂-C₆alkynyl, C₂-C₆alkanoyl, C₂-C₆alkyloxime, C₁-C₆alkoxy, (C₁-C₆alkoxy)C₁-C₄alkyl, hydroxyC₁-C₆alkyl, C₁-C₆alkoxycarbonyl, mono- or di-C₁-C₆alkylaminocarbonyl, C₁-C₆alkylthio, C₁-C₆alkylsulfonyl, haloC₁-C₆alkyl, haloC₁-C₆alkoxy, aminoC₁-C₆alkyl, mono- or di-(C₁-C₆alkyl)aminoC₀-C₆alkyl, or (C₃-C₇cycloalkyl)C₀-C₆alkyl; or
R₂ is (4- to 7-membered heterocycloalkyl)C₀-C₆alkyl, phenylC₀-C₂alkyl, phenylC₀-C₂alkoxy, or (5- or 6-membered heteroaryl)C₀-C₂alkyl, each of which is substituted with from 0 to 3 substituents independently chosen from halogen, C₁-C₂alkoxy and C₁-C₂alkyl; or
R₂ is taken together with a R₁ to form a fused optionally substituted 5- or 6-membered carbocycle or heterocycle;
n is 1 or 2;
R₃ is:
(i) hydrogen, C₁-C₆alkyl, C₂-C₆alkenyl or haloC₁-C₆alkyl; or
(ii) taken together with one or both of R₆ and R₁₀ to form a fused carbocycle or heterocycle having one or two rings, wherein each ring contains from 5 to 8 ring members and 0, 1 or 2 heteroatoms independently chosen from N, O and S, which fused carbocycle or heterocycle is substituted with from 0 to 3 substituents independently chosen from halogen, oxo, C₁-C₂alkoxy, and C₁-C₂alkyl;
R₄ is hydrogen, C₁-C₆alkyl, or haloC₁-C₆alkyl;
R₅ is:
(i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino or aminoC₁-C₆alkyl; or
(ii) taken together with R₆ to form a fused C₅-C₈carbocycle or 5- to 8-membered heterocycle;
Each R₅ₐ is independently hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino, or aminoC₁-C₆alkyl;
R₆ is:
(i) taken together with R₃ to form a fused, optionally substituted heterocycle; or
(ii) taken together with R₅ to form a fused carbocycle or heterocycle;
P is N or CR₇;
Q is N or CR₈;
U is N or CR₉;
T is N or CR₁₀;
R₇ is:
(i) hydrogen, halogen, nitro, cyano, —COOH, or a group of the formula M-L-;
(ii) taken together with R₈ to form a fused C₅-C₈carbocycle or a fused 5- to 8-membered heterocycle; or
(iii) taken together with R₁₂ to form a fused 5- or 6-membered heterocycle that is substituted with from 0 to 3 substituents independently chosen from halogen, C₁-C₂alkyl, C₁-C₂alkoxy, and oxo;
R₈ is:
(i) hydrogen, halogen, nitro, cyano, —COOH, or a group of the formula M-L-; or
(ii) taken together with R₇ to form a fused C₅-C₈carbocycle or a fused 5- to 8-membered heterocycle;
R₉ is:
(i) hydrogen, halogen, nitro, cyano, —COOH, or a group of the formula M-L-; or
(ii) taken together with R₁₀ or R₁, to form a fused C₅-C₁₀carbocycle or a fused 5- to 10-membered heterocycle, each of which is substituted with from 0 to 3 substituents independently chosen from halogen, amino, nitro, cyano, hydroxy, oxo, acetyl, aminocarbonyl, imino, C₁-C₆alkyl, C₂-C₆alkenyl, C₂-C₆alkynyl, (C₃-C₇cycloalkyl)C₀-C₆alkyl, C₁-C₆alkoxy, C₁-C₆alkylthio, C₁-C₆alkylsulfonyl, (C₁-C₆alkoxy)C₁-C₄alkyl, (C₁-C₆alkoxy)C₁-C₆alkoxy, mono- and di-(C₁-C₆alkyl)aminoC₀-C₆alkyl, C₂-C₆alkanoyl, C₁-C₆alkoxycarbonyl, mono- or di-(C₁-C₆alkyl)aminocarbonyl, haloC₁-C₆alkyl, hydroxyC₁-C₆alkyl, aminoC₁-C₆alkyl, and haloC₁-C₆alkoxy;
R₁₀ is:
(i) hydrogen, halogen, nitro, cyano, —COOH, or a group of the formula M-L-; or
(ii) taken together with R₃ or R₉ to form a fused, optionally substituted carbocycle or heterocycle;
R₁₁ is:
(i) a group of the formula G-L-, wherein G is C₁-C₆alkyl, C₂-C₆alkenyl, C₂-C₆alkynyl, haloC₁-C₆alkyl, saturated C₃-C₁₀cycloalkyl, or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ, R_{b} and R_{c}; wherein
Rₐ is oxo, oxime, hydroxy, cyano, —COOH, —(C═O)NH₂, —NH(C═O)H, —SO₂NH₂, —(C═N)OH or imino;
R_{b} is C₁-C₆alkoxy, (C₁-C₆alkoxy)C₁-C₆alkoxy, mono- or di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkylsulfonylamino, C₁-C₆alkoxycarbonyl, C₂-C₆alkanoylamino, arylC₁-C₆alkanoylamino, heteroarylC₁-C₆alkanoylamino, mono- or di-(C₁-C₆alkyl)aminocarbonyl, or C₁-C₆alkyloxime, each of which is substituted with from 0 to 5 substituents independently chosen from halogen, amino, cyano, hydroxy, oxo, oxime, C₁-C₄alkyl, (C₁-C₄alkoxy)C₀-C₄alkyl, mono- and di-(C₁-C₄alkyl)amino, C₂-C₄alkanoyl, C₃-C₇cycloalkyl, C₁-C₄alkoxycarbonyl, haloC₁-C₂alkyl or haloC₁-C₂alkoxy; and
R_{c} is carbocycleC₀-C₆alkyl, heterocycleC₀-C₆alkyl, carbocycleC₀-C₆alkoxy, heterocycleC₀-C₆alkoxy, carbocycleC₀-C₆alkylamino, or heterocycleC₀-C₆alkylamino, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl, and haloC₁-C₆alkoxy;
(ii) C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl, and haloC₁-C₆alkoxy; or
(iii) taken together with R₉ to form a fused optionally substituted carbocycle or heterocycle;
R₁₂ is:
(i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino, or aminoC₁-C₆alkyl; or
(ii) taken together with R₇ to form a fused, optionally substituted heterocycle;
each L is independently a single covalent bond, N(R₁₃), O, S, C(═O), C(═O)O, OC(═O), SO, SO₂, SO₂N(R₁₃), N(R₁₃)SO₂, C(═O)N(R₁₃), or N(R₁₃)C(═O), wherein each R₁₃ is independently hydrogen, C₁-C₆alkyl, C₂-C₆alkenyl, C₂-C₆alkynyl, or haloC₁-C₆alkyl; and
each M is independently hydrogen, C₁-C₆alkyl, C₂-C₆alkenyl, C₂-C₆alkynyl, haloC₁-C₆alkyl, hydroxyC₁-C₆alkyl, aminoC₁-C₆alkyl, (C₁-C₆alkoxy)C₁-C₆alkyl, C₅-C₁₀cycloalkyl, or 5- to 10-membered heterocycloalkyl.

3. A compound of the formula: 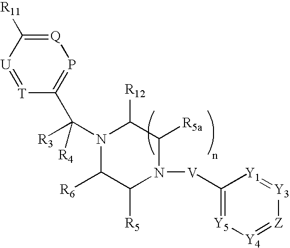
or a pharmaceutically acceptable salt thereof, wherein:
V is absent or —(C═O);
Y₁, Y₃, Y₄ and Y₅ are independently CR₁ or nitrogen;
Z is nitrogen or CR₂;
each R₁ is independently:
(i) hydrogen, halogen, hydroxy, nitro, cyano, amino, aminocarbonyl, C₁-C₆alkyl, C₂-C₆alkenyl, C₂-C₆alkynyl, C₁-C₆alkoxy, haloC₁-C₆alkyl, haloC₁-C₆alkoxy, hydroxyC₁-C₆alkyl, (C₁-C₄alkoxy)C₁-C₄alkyl, C₁-C₆alkylthio, aminoC₁-C₆alkyl, mono- or di-(C₁-C₆alkyl)aminoC₀-C₆alkyl, mono- or di-(C₁-C₆alkyl)aminocarbonyl, (C₃-C₇cycloalkyl)C₀-C₆alkyl, or (4- to 7-membered heterocycloalkyl)C₀-C₆alkyl; or
(ii) taken together with R₂ to form a fused 5- or 6-membered carbocycle or heterocycle, each of which is substituted with from 0 to 3 substituents independently chosen from halogen, hydroxy, nitro, cyano, amino, C₁-C₄alkyl, C₁-C₄alkoxy, haloC₁-C₄alkyl, and haloC₁-C₄alkoxy;
R₂ is halogen, hydroxy, nitro, cyano, amino, acetyl, aminocarbonyl, imino, C₁-C₆alkyl, C₂-C₆alkenyl, C₂-C₆alkynyl, C₂-C₆alkanoyl, C₂-C₆alkyloxime, C₁-C₆alkoxy, (C₁-C₆alkoxy)C₁-C₄alkyl, hydroxyC₁-C₆alkyl, C₁-C₆alkoxycarbonyl, mono- or di-C₁-C₆alkylaminocarbonyl, C₁-C₆alkylthio, C₁-C₆alkylsulfonyl, haloC₁-C₆alkyl, haloC₁-C₆alkoxy, aminoC₁-C₆alkyl, mono- or di-(C₁-C₆alkyl)aminoC₀-C₆alkyl, or (C₃-C₇cycloalkyl)C₀-C₆alkyl; or
R₂ is (4- to 7-membered heterocycloalkyl)C₀-C₆alkyl, phenylC₀-C₂alkyl, phenylC₀-C₂alkoxy, or (5- or 6-membered heteroaryl)C₀-C₂alkyl, each of which is substituted with from 0 to 3 substituents independently chosen from halogen, C₁-C₂alkoxy, and C₁-C₂alkyl; or
R₂ is taken together with a R₁ to form a fused optionally substituted 5- or 6-membered carbocycle or heterocycle;
n is 1 or 2;
R₃ is:
(i) hydrogen, C₁-C₆alkyl, C₂-C₆alkenyl, or haloC₁-C₆alkyl; or
(ii) taken together with one or both of R₆ and R₁₀ to form a fused carbocycle or heterocycle having one or two rings, wherein each ring contains from 5 to 8 ring members and 0, 1, or 2 heteroatoms independently chosen from N, Q and S, which fused carbocycle or heterocycle is substituted with from 0 to 3 substituents independently chosen from halogen, oxo, C₁-C₂alkoxy, and C₁-C₂alkyl;
R₄ is hydrogen, C₁-C₆alkyl, or haloC₁-C₆alkyl;
R₅ is:
(i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino, or aminoC₁-C₆alkyl; or
(ii) taken together with R₆ to form a fused C₅-C₈carbocycle or a fused 5- to 8-membered heterocycle;
each R₅ₛ is independently:
(i) hydrogen, halogen, hydroxy, nitro, cyano, amino, C₁-C₆alkyl, C₂-C₆alkenyl, C₂-C₆alkynyl, C—C₆alkoxy, haloC₁-C₆alkyl, haloC₁-C₆alkoxy, mono- or di-(C₁-C₆alkyl)amino, or aminoC₁-C₆alkyl; or
(ii) taken together with R₆ to form a methylene or ethylene bridge;
R₆ is:
(i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino, or aminoC₁-C₆alkyl;
(ii) taken together with R₃ to form a fused, optionally substituted heterocycle;
(iii) taken together with R₅ to form a fused carbocycle or heterocycle; or
(iv) taken together with R₅ₐ to form a methylene or ethylene bridge;
P is N or CR₇;
Q is N or CR₈;
U is N or CR₉;
T is N or CR₁₀;
R₇ is:
(i) hydrogen, halogen, nitro, cyano, —COOH, or a group of the formula M-L-;
(ii) taken together with R₈ to form a fused C₅-C₈carbocycle or a fused 5- to 8-membered heterocycle; or
(iii) taken together with R₁₂ to form a fused 5- or 6-membered heterocycle that is substituted with from 0 to 3 substituents independently chosen from halogen, C₁-C₂alkyl, C₁-C₂alkoxy, and oxo;
R₈ is:
(i) hydrogen, halogen, nitro, cyano, —COOH, or a group of the formula M-L-; or
(ii) taken together with R₇ to form a fused C₅-C₆carbocycle or a fused 5- to 6-membered heterocycle;
R₉ is:
(i) hydrogen, halogen, nitro, cyano, —COOH or a group of the formula M-L-; or
(ii) taken together with R₁₀ or R₁₁ to form a fused C₅-C₁₀carbocycle or a fused 5- to 10-membered heterocycle, each of which is substituted with from 0 to 3 substituents independently chosen from halogen, amino, nitro, cyano, hydroxy, oxo, acetyl, aminocarbonyl, imino, C₁-C₆alkyl, C₂-C₆alkenyl, C₂-C₆alkynyl, (C₃-C₇cycloalkyl)C₀-C₆alkyl, C₁-C₆alkoxy, C₁-C₆alkylthio, C₁-C₆alkylsulfonyl, (C₁-C₆alkoxy)C₁-C₄alkyl, (C₁-C₆alkoxy)C₁-C₆alkoxy, mono- and di-(C₁-C₆alkyl)aminoC₀-C₆alkyl, C₂-C₆alkanoyl, C₁-C₆alkoxycarbonyl, mono- or di-(C₁-C₆alkyl)aminocarbonyl, haloC₁-C₆alkyl, hydroxyC₁-C₆alkyl, aminoC₁-C₆alkyl, and haloC₁-C₆alkoxy;
R₁₀ is:
(i) hydrogen, halogen, nitro, cyano, —COOH or a group of the formula M-L-; or
(ii) taken together with R₃ or R₉ to form a fused, optionally substituted carbocycle or heterocycle;
R₁₁ is:
(i) a group of the formula G-L₁-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, and wherein G is further substituted with from 1 to 5 substituents independently chosen from Rₐ, R_{b} and R_{c};
(ii) a group of the formula G₁-O— wherein G₁ is C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G₁ is further substituted with from 1 to 5 substituents independently chosen from Rₐ, R_{b} and R_{c};
(iii) a group of the formula G₂-O— wherein G₂ is C₁-C₆alkyl that is substituted with from 0 to 3 amino groups, and wherein G₂ is further substituted with from 1 to 5 substituents independently chosen from Rₐ, R_{b} and R_{c}; such that Rb is not N-methyl, N-cyclopentylamino, and R_{c} is not (heterocycle)C₀-C₆alkyl;
(iv) C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy; or
(v) taken together with R₉ to form a fused optionally substituted carbocycle or heterocycle;
Rₐ is oxo, oxime, hydroxy, cyano, —COOH, —(C═O)NH₂, —NH(C═O)H, —SO₂NH₂, —(C═N)OH or imino;
R_{b} is C₁-C₆alkoxy, (C₁-C₆alkoxy)C₁-C₆alkoxy, mono- or di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkysulfonylamino, C₁-C₆alkoxycarbonyl, C₂-C₆alkanoylamino, arylC₁-C₆alkanoylamino, heteroarylC₁-C₆alkanoylamino, mono- or di-(C₁-C₆alkyl)aminocarbonyl or C₁-C₆alkyloxime, each of which is substituted with from 0 to 5 substituents independently chosen from halogen, amino, cyano, hydroxy, oxo, oxime, C₁-C₄alkyl, (C₁-C₄alkoxy)C₀-C₄alkyl, mono- and di-(C₁-C₄alkyl)amino, C₂-C₄alkanoyl, C₃-C₇cycloalkyl, C₁-C₄alkoxycarbonyl, haloC₁-C₂alkyl and haloC₁-C₂alkoxy;
R_{c} is carbocycleC₀-C₆alkyl, heterocycleC₀-C₆alkyl, carbocycleC₀-C₆alkoxy, heterocycleC₀-C₆alkoxy, carbocycleC₀-C₆alkylamino or heterocycleC₀-C₆alkylamino; each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy;
R₁₂ is:
(i) hydrogen, halogen, hydroxy, nitro, cyano, amino, C₁-C₆alkyl, C₂-C₆alkenyl, C₂-C₆alkynyl, C₁-C₆alkoxy, haloC₁-C₆alkyl, haloC₁-C₆alkoxy, mono- or di-(C₁-C₆alkyl)amino, or aminoC₁-C₆alkyl; or
(ii) taken together with R₇ to form a fused, optionally substituted heterocycle;
each L is independently a single covalent bond, N(R₁₃), O, S, C(═O), C(═O)O, OC(═O), SO, SO₂, SO₂N(R₁₃), N(R₁₃)SO₂, C(═O)N(R₁₃) or N(R₁₃)C(═O);
L₁ is independently a single covalent bond, N(R₁₃), C(═O), C(═O)O, OC(═O), SO₂, SO₂N(R₁₃), N(R₁₃)SO₂, C(═O)N(R₃) or N(R₁₃)C(═O);
each R₁₃ is independently hydrogen, C₁-C₆alkyl, C₂-C₆alkenyl, C₂-C₆alkynyl or haloC₁-C₆alkyl; and
each M is independently hydrogen, C₁-C₆alkyl, C₂-C₆alkenyl, C₂-C₆alkynyl, haloC₁-C₆alkyl, hydroxyC₁-C₆alkyl, aminoC₁-C₆alkyl, (C₁-C₆alkoxy)C₁-C₆alkyl, C₅-C₁₀cycloalkyl or 5- to 10-membered heterocycloalkyl.

4-5. (canceled)

6. A compound or salt of claim 3, wherein V is absent.

7. A compound or salt of claim 3, wherein V is —(C═O)—.

8. A compound or salt of claim 3, wherein n is 1.

9-13. (canceled)

14. A compound or salt of claim 3, wherein the compound has the formula: 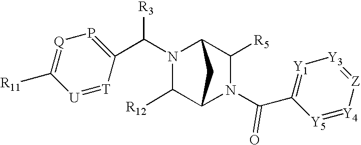
wherein:
R₃ is hydrogen, C₁-C₂alkyl, or haloC₁-C₂alkyl;
R₅ is hydrogen, C₁-C₂alkyl, or C₁-C₂alkoxy; and
R₁₂ is hydrogen, C₁-C₂alkyl, or C₁-C₂alkoxy.

15. A compound or salt of claim 2, wherein the compound has the formula: 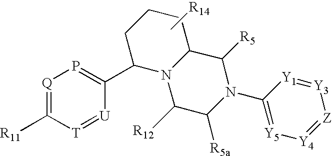
wherein:
R₅, R₅ₐ, and R₁₂ are each independently hydrogen, C₁-C₂alkyl, or C₁-C₂alkoxy; and
R₁₄ represents from 0 to 3 substituents independently chosen from halogen, C₁-C₂alkyl, C₁-C₂alkoxy, and oxo.

16. A compound or salt of claim 2, wherein the compound has the formula: 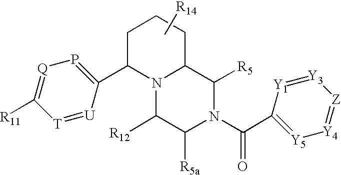
wherein:
R₅, R₅ₐ, and R₁₂ are each independently hydrogen, C₁-C₂alkyl, or C₁-C₂alkoxy; and
R₁₄ represents from 0 to 3 substituents independently chosen from halogen, C₁-C₂alkyl, C₁-C₂alkoxy, and oxo.

17-19. (canceled)

20. A compound or salt of claim 3, wherein Y₁, Y₄ and Y₅ are CH, Y₃ is CR₁, and Z is CR₂.

21. A compound or salt of claim 3, wherein Y₁ is nitrogen, Y₃, Y₄ and Y₅ are CR₁, and Z is CR₂.

22. A compound or salt of claim 3, wherein Y₁ and Y₄ are nitrogen, Y₃ and Y₅ are CR₁, and Z is CR₂.

23. A compound or salt of claim 3, wherein Y₄ is nitrogen, Y₁, Y₃, and Ys are CR₁, and Z is CR₂.

24-25. (canceled)

26. A compound or salt of claim 3, wherein each R₁ is independently hydrogen, halogen, C₁-C₂alkyl, C₁-C₂alkoxy, or trifluoromethyl.

27-28. (canceled)

29. A compound or salt of claim 3, wherein R₂ is halogen, C₁-C₄alkyl, C₁-C₄alkoxy, or trifluoromethyl.

30. A compound or salt of claim 29, wherein each R₁ is hydrogen and R₂ is trifluoromethyl.

31-33. (canceled)

34. A compound or salt of claim 3 wherein R₃ is methyl and R₄ is hydrogen.

35-40. (canceled)

41. A compound or salt of claim 3, wherein P is CR₇, Q is CR₈, U is CR₉, and T is CR₁₀.

42-44. (canceled)

45. A compound or salt of claim 3, wherein R₇, R₈, R₉, and R₁₀ are each independently hydrogen, halogen, C₁-C₂alkyl, C₁-C₂alkoxy, haloC₁-C₂alkyl, or haloC₁-C₂alkoxy.

46-48. (canceled)

49. A compound or salt of claim 45, wherein R₇ and R₈ are both methyl, and R₉ and R₁₀ are both hydrogen.

50. A compound or salt of claim 3, wherein R₁₁ is a group of the formula G-L-.

51. (canceled)

52. A compound or salt of claim 50, wherein G is C₁-C₆alkyl, C₂-C₆alkenyl, or C₂-C₆alkynyl, each of which is substituted with from 0 to 3 substituents independently chosen from halogen and amino, and wherein G is substituted with from 1 to 5 substituents independently chosen from Rₐ and R_{b}.

53-54. (canceled)

55. A compound or salt of claim 50, wherein:
G is substituted with at least one substituent chosen from Rₐ and R_{b}; and
R_{b} is C₁-C₆alkoxy, (C₁-C₆alkoxy)C₁-C₆alkoxy, mono- or di-(C₁-C₈alkyl)aminoC₀-C₆alkyl, C₂-C₆alkanoyl, C₁-C₆alkylsulfonyl, C₁-C₆alkylthio, C₁-C₆alkylaminosulfonyl, C₁-C₆alkysulfonylamino, C₁-C₆alkoxycarbonyl, C₂-C₆alkanoylamino, mono- or di-(C₁-C₆alkyl)aminocarbonyl, or C₁-C₆alkyloxime, each of which is substituted with from 0 to 5 substituents independently chosen from halogen, amino, cyano, hydroxy, oxo, oxime, C₁-C₄alkyl, (C₁-C₄alkoxy)C₀-C₄alkyl, mono- and di-(C₁-C₄alkyl)amino, C₂-C₄alkanoyl, C₃-C₇cycloalkyl, C₁-C₄alkoxycarbonyl, haloC₁-C₂alkyl and haloC₁-C₂alkoxy.

56. A compound or salt of claim 55, wherein:
G is C₁-C₆alkyl, substituted with from 0 to 3 substituents independently chosen from halogen and amino; wherein G is substituted with from 1 to 5 substituents independently chosen from:
(a) oxo, oxime, hydroxy, cyano, —(C═O)NH₂, —NH(C═O)H, or imino; and
(b) C₁-C₆alkoxy, mono- or di-(C₁-C₈alkyl)amino, C₁-C₆alkoxycarbonyl, or C₂-C₆alkanoylamino, each of which is substituted with from 0 to 5 substituents independently chosen from halogen, oxo, C₁-C₄alkoxy, mono- and di-C₁-C₄alkylamino, C₂-C₄alkanoyl, C₃-C₇cycloalkyl, haloC₁-C₂alkyl, and haloC₁-C₂alkoxy.

57-67. (canceled)

68. A compound or salt of claim 50, wherein R₁₁ is a group of the formula G-L-, where L is O.

69-83. (canceled)

84. A compound or salt of claim 3, wherein:
R₁₁ is a group of the formula G₂-O—; and
G₂ is C₁-C₆alkyl that is substituted with from 0 to 3 substituents independently chosen from halogen and amino, wherein G₂ is further substituted with from 1 to 5 substituents independently chosen from Rₐ, R_{b} and R_{c}, such that R_{b} is not N-methyl,N-cyclopentylamino, and R_{c} is not (heterocycle)C₀-C₆alkyl.

85. (canceled)

86. A compound or salt of claim 84, wherein G₂ is substituted with from 0 to 3 substituents independently chosen from halogen and amino, and wherein G₂ is substituted with from 1 to 5 substituents independently chosen from Rₐ and R_{b} wherein:
Rₐ is oxo, hydroxy, cyano, —(C═O)NH₂, —NH(C═O)H, or imino; and
R_{b} is C₁-C₆alkoxy, mono- and di-(C₁-C₈alkyl)amino, C₁-C₆alkoxycarbonyl, or C₂-C₆alkanoylamino, each of which is substituted with from 0 to 5 substituents independently chosen from halogen, oxo, C₁-C₄alkoxy, mono- and di-C₁-C₄alkylamino, C₂-C₄alkanoyl, C₃-C₇cycloalkyl, haloC₁-C₂alkyl and haloC₁-C₂alkoxy.

87-90. (canceled)

91. A compound of claim 3, wherein the compound satisfies the formula:

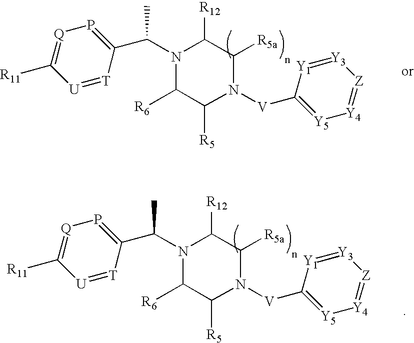

92. A compound or salt of claim 91, wherein the compound satisfies the formula:

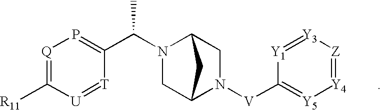

93. A compound or salt of claim 92, wherein the compound satisfies the formula: 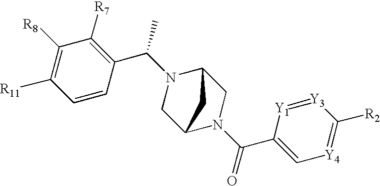
wherein:
each R₁ is hydrogen or methoxy;
R₂ is chloro, fluoro or trifluoromethyl;
R₇ and R₈ are independently hydrogen, halogen, hydroxy, nitro, cyano, —COOH or a group of the formula M-L-; and
R₁₁ is:
(i) a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is substituted with from 1 to 5 substituents independently chosen from Rₐ, R_{b} and R_{c}; or
(ii) C₅-C₁₀cycloalkenyl, phenyl, naphthyl, 5- to 10-membered heterocycloalkenyl or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl, and haloC₁-C₆alkoxy.

94. A compound or salt of claim 92, wherein:
R₇ and R₈ are independently hydrogen, halogen, C₁-C₂alkyl or haloC₁-C₂alkyl; and
R₁₁ is a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is substituted with from 1 to 5 substituents independently chosen from Rₐ and R_{b}.

95-97. (canceled)

98. A compound or salt of claim 3, wherein the compound satisfies the formula: 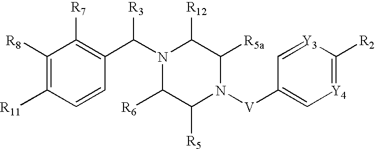
wherein:
each R₁ is hydrogen or methoxy;
R₂ is chloro, fluoro, or trifluoromethyl;
R₃ is:
(i) hydrogen or methyl; or
(ii) taken together with R₆ to form a fused 5- to 7-membered heterocycloalkyl that has 0 or 1 additional heteroatoms chosen from N, S and O, which fused 5- to 7-membered heterocycloalkyl is substituted with from 0 to 2 substituents independently chosen from halogen, oxo, C₁-C₂alkoxy and C₁-C₂alkyl;
R₅ is hydrogen, methyl or methoxy;
R₅ₐ is:
(i) hydrogen, methyl or methoxy;
(ii) taken together with R₃ to form a methylene or ethylene bridge;
R₆ is:
(i) hydrogen, methyl, or methoxy;
(ii) taken together with R₃ to form a fused, optionally substituted, 5- to 7-membered heterocycloalkyl; or
(iii) taken together with R₅ₐ to form a methylene or ethylene bridge;
R₇ and R₈ are independently hydrogen, halogen, hydroxy, nitro, cyano, —COOH or a group of the formula M-L-;
R₁₁ is:
(i) a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is substituted with from 1 to 5 substituents independently chosen from Rₐ, R_{b} and R_{c}; or
(ii) C₅-C₁₀cycloalkenyl, phenyl, naphthyl, 5- to 10-membered heterocycloalkenyl, or 5- to 10-membered heteroaryl, each of which is substituted with from 0 to 5 substituents independently chosen from halogen, amino, cyano, hydroxy, oxo, C₁-C₆alkyl, (C₁-C₆alkoxy)C₀-C₆alkoxy, mono- and di-(C₁-C₆alkyl)aminoC₀-C₆alkyl, C₂-C₄alkanoyl, (C₃-C₇cycloalkyl)C₀-C₆alkyl, C₁-C₄alkoxycarbonyl, haloC₁-C₆alkyl and haloC₁-C₆alkoxy; and
R₁₂ is hydrogen, methyl, or methoxy.

99. A compound or salt of claim 98, wherein the compound satisfies the formula: 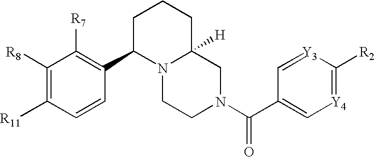
wherein:
R₇ and R₈ are independently hydrogen, halogen, C₁-C₂alkyl, or haloC₁-C₂alkyl; and
R₁₁ is a group of the formula G-L-, wherein G is C₁-C₆alkyl, C₂-C₆alkenyl, C₂-C₆alkynyl, haloC₁-C₆alkyl, saturated C₃-C₁₀cycloalkyl or saturated 3- to 10-membered heterocycloalkyl; each of which is substituted with from 0 to 3 substituents independently chosen from halogen, amino and C₁-C₆alkyl, wherein G is further substituted with from 1 to 5 substituents independently chosen from Rₐ and R_{b}.

100-103. (canceled)

104. A pharmaceutical composition, comprising a compound or salt of claim 3, in combination with at least one physiologically acceptable carrier or excipient.

105-115. (canceled)

116. A method for treating a disease or disorder associated with MCH receptor activation, comprising administering to a patient in need of such treatment a therapeutically effective amount of a compound of claim 3.

117. The method of claim 116, wherein the disease or disorder is an eating disorder, sexual disorder, diabetes, heart disease or stroke.

118. The method of claim 117, wherein the compound or salt is administered orally.

119. The method of claim 117, wherein the compound or salt is administered intranasally, intravenously or topically.

120. The method of claim 117, wherein the patient is a human.

121. The method of claim 117, wherein the patient is a dog or a cat.

122. A method for treating obesity, comprising administering to a patient in need of such treatment a therapeutically effective amount of a compound of claim 3.

123. The method of claim 122, wherein the compound or salt is administered orally.

124. The method of claim 122, wherein the patient is a human.

125. The method of claim 122, wherein the patient is a dog or a cat.

126-130. (canceled)

131. A packaged pharmaceutical preparation, comprising: (i) a pharmaceutical composition of claim 104 in a container; and (ii) instructions for using the composition to treat a patient suffering from a disorder associated with MCH receptor activation.

132-134. (canceled)

135. A compound or salt according to claim 3, wherein the compound is:
{(6R,9aS)-6-[4-(2-methoxy-ethoxy)-2,3-dimethylphenyl]-octahydro-pyrido[1,2-a]pyrazin-2-yl}-(6-trifluoromethyl-pyridin-3-yl)-methanone;
{(6R,9aS)-6-[4-(2-hydroxy-ethoxy)-2,3-dimethyl-phenyl]-octa-hydro-pyrido[1,2-a]pyrazin-2-yl}-(6-trifluoromethyl-pyridin-3-yl)-methanone;
{(6R,9aS)-6-[4-((S)-2-Hydroxy-propoxy)-2,3-dimethyl-phenyl]-octahydro-pyrido[1,2-a]pyrazin-2-yl}-(6-trifluoromethyl-pyridin-3-yl)-methanone;
{(6R,9aS)-6-[4-((R)-2-Hydroxy-propoxy)-2,3-dimethyl-phenyl]-octahydro-pyrido[1,2-a]pyrazin-2-yl}-(6-trifluoromethyl-pyridin-3-yl)-methanone;
1-{2,3-Dimethyl-4-[(6R,9aS)-2-(6-trifluoromethyl-pyridine-3-carbonyl)-octahydro-pyrido[1,2-a]pyrazin-6-yl]-phenoxy}-propan-2-one;
1-{2,3-Dimethyl-4-[(6R,9aS)-2-(6-trifluoromethyl-pyridine-3-carbonyl)-octahydro-pyrido[1,2-a]pyrazin-6-yl]-phenoxy}-propan-2-one oxime;
(6-Chloropyridin-3-yl)-((1S,4S)-5-{(S)-1-[4-(2-methoxy-ethoxy)-2,3-dimethylphenyl]-ethyl}-2,5-diazabicyclo[2.2.1]hept-2-yl)-methanone;
(6-Ethylpyridin-3-yl)-((1S,4S)-5-{(S)-1-[4-(2-methoxy-ethoxy)-2,3-dimethylphenyl]-ethyl}-2,5-diazabicyclo[2.2.1]hept-2-yl)-methanone;
[(6R,8aS)-6-(4-methoxy-2,3-dimethylphenyl)-hexahydro-pyrrolo[1,2-a]pyrazin-2-yl]-(6-trifluoromethyl-pyridin-3-yl)-methanone;
N-(3-{4-[(6R,9aS)-2-(4-chloro-3-methoxy-phenyl)-octahydro-pyrido[1,2-a]pyrazin-6-yl]-2,3-dimethylphenoxy}-propyl)-acetamide;
N-(3-{4-[(6R,9aS)-2-(4-fluoro-3-methoxy-phenyl)-octahydro-pyrido[1,2-a]pyrazin-6-yl]-2,3-dimethylphenoxy}-propyl)-acetamide;
4-(4-Chloro-3-trifluoromethyl-phenyl)-1-[4-(2-methoxy-ethoxy)-2,3-dimethyl-benzyl]-piperidin-4-ol;
2-[2,3-dimethyl-4-(1-{4-[4-(trifluoromethyl)benzoyl]piperazin-1-yl}ethyl)phenoxy]-N,N-dimethylethanamine;
1-{1-[4-(2-methoxyethoxy)-2,3-dimethylphenyl]ethyl}-4-[4-(trifluoromethyl)benzoyl]piperazine;
2-[2,3-dimethyl-4-((1R)-1-{4-[4-(trifluoromethyl)benzoyl]piperazin-1-yl}ethyl)phenoxy]-N,N-dimethylethanamine;
3-[2,3-dimethyl-4-(1-{4-[4-(trifluoromethyl)benzoyl]piperazin-1-yl}ethyl)phenoxy]-N,N-dimethylpropan-1-amine;
1-(4-chlorobenzoyl)-4-{1-[4-(2-methoxyethoxy)-2,3-dimethylphenyl]ethyl}piperazine;
1-(4-chlorobenzoyl)-4-{1-[4-(2-ethoxyethoxy)-2,3-dimethylphenyl]ethyl}piperazine;
ethyl (4-{1-[4-(4-chlorobenzoyl)piperazin-1-yl]ethyl}-2,3-dimethylphenoxy)acetate;
1-(4-{1-[4-(4-chlorobenzoyl)piperazin-1-yl]ethyl}-2,3-dimethylphenoxy)-2-methylpropan-2-ol;
1-(4-chlorobenzoyl)-4-{(1R)-1-[4-(2-methoxyethoxy)-2,3-dimethylphenyl]ethyl}piperazine;
3-(4-{1-[4-(4-chlorobenzoyl)piperazin-1-yl]ethyl}-2,3-dimethylphenoxy)-N,N-dimethylpropan-1-amine;
2-(4-{1-[4-(4-chlorobenzoyl)piperazin-1-yl]ethyl}-2,3-dimethylphenoxy)ethanol;
2-(4-{1-[4-(4-chlorobenzoyl)piperazin-1-yl]ethyl}-2,3-dimethylphenoxy)-N-methylethanamine;
2-(4-{1-[4-(4-chlorobenzoyl)piperazin-1-yl]ethyl}-2,3-dimethylphenoxy)ethanamine;
(1S,4S)-2-{(1R)-1-[4-(2-methoxyethoxy)-2,3-dimethylphenyl]ethyl}-5-[4-(trifluoromethyl)benzoyl]-2,5-diazabicyclo[2.2.1]heptane;
1-(4-chlorobenzoyl)-4-((1R)-1-{2,3-dimethyl-4-[2-(1-methylpyrrolidin-2-yl)ethoxy]phenyl}ethyl)piperazine;
2-[2,3-dimethyl-4-(1-{4-[4-(trifluoromethyl)benzoyl]piperazin-1-yl}ethyl)phenoxy]-N,N-dimethylethanamine;
3-[2,3-dimethyl-4-((1S)-1-{4-[4-(trifluoromethyl)benzoyl]-piperazin-1-yl}ethyl)phenoxy]-N,N-dimethylpropan-1-amine;
3-[2,3-dimethyl-4-((1R)-1-{4-[4-(trifluoromethyl)benzoyl]-piperazin-1-yl}ethyl)phenoxy]-N,N-dimethylpropan-1-amine;
1-(4-chlorobenzoyl)-4-(1-{2,3-dimethyl-4-[3-(methylthio)-propoxy]phenyl}ethyl)piperazine;
1-(4-chlorobenzoyl)-4-(1-{2,3-dimethyl-4-[3-(methylsulfonyl)-propoxy]phenyl}ethyl)piperazine;
3-(4-{1-[4-(4-chlorobenzoyl)piperazin-1-yl]ethyl}-2,3-dimethylphenoxy)-N-methylpropan-1-amine;
3-[2,3-dimethyl-4-((1S)-1-{(1S,4S)-5-[4-(trifluoromethyl)benzoyl]-2,5-diazabicyclo[2.2.1]hept-2-yl}ethyl)phenoxy]-N,N-dimethylpropan-1-amine;
(1R,4R)-2-(4-chlorobenzoyl)-5-{(1R)-1-[4-(2-methoxyethoxy)-2,3-dimethylphenyl]ethyl}-2,5-diazabicyclo[2.2.1]heptane;
3-[2,3-dimethyl-4-((S)-1-{(1S,4S)-5-[4-(trifluoromethyl)benzoyl]-2,5-diazabicyclo[2.2.1]hept-2-yl}ethyl)phenoxy]-N-methylpropan-1-amine;
3-[2,3-dimethyl-4-((1R)-1-{(1S,4S)-5-[4-(trifluoromethyl)benzoyl]-2,5-diazabicyclo[2.2.1]hept-2-yl}ethyl)phenoxy]-N,N-dimethylpropan-1-amine;
3-(4-{1-[4-(4-chloro-benzoyl)piperazin-1-yl]ethyl}-2,3-dimethylphenoxy)propan-1-ol;
(1R,4R)-2-{(1R)-1-[4-(3-methoxypropoxy)-2,3-dimethylphenyl]ethyl}-5-[4-(trifluoromethyl)benzoyl]-2,5-diazabicyclo[2.2.1]heptane;
(1R,4R)-2-{(1S)-1-[4-(3-methoxypropoxy)-2,3-dimethylphenyl]ethyl}-5-[4-(trifluoromethyl)benzoyl]-2,5-diazabicyclo[2.2.1]heptane;
(1R,4R)-2-(4-chlorobenzoyl)-5-{(1R)-1-[4-(3-methoxypropoxy)-2,3-dimethylphenyl]ethyl}-2,5-diazabicyclo[2.2.1]heptane;
1-(4-chlorobenzoyl)-4-{1-[4-(3-chloropropoxy)-2,3-dimethylphenyl]ethyl}piperazine
3-{4-[(6R,9aS)-2-(4-chlorobenzoyl)octahydro-2H-pyrido[1,2-a]pyrazin-6-yl]-2,3-dimethylphenoxy}-N,N-dimethylpropan-1-amine;
(1S,4S)-2-{(1S)-1-[4-(difluoromethoxy)-2,3-dimethylphenyl]ethyl}-5-[4-(trifluoromethyl)benzoyl]-2,5-diazabicyclo[2.2.1]heptane;
3-(4-{(1S)-1-[(1S,4S)-5-(4-chlorobenzoyl)-2,5-diazabicyclo[2.2.1]hept-2-yl]ethyl}-2,3-dimethylphenoxy)-N-methylpropan-1-amine;
N-[3-(4-{(1S)-1-[(1S,4S)-5-(4-chlorobenzoyl)-2,5-diazabicyclo[2.2.1]hept-2-yl]ethyl}-2,3-dimethylphenoxy)propyl]-N-methylacetamide;
3-[2,3-dimethyl-4-((1R)-1-{4-[4-(trifluoromethyl)benzoyl]piperazin-1-yl}ethyl)phenoxy]propan-1-amine;
2-[2,3-dimethyl-4-((1R)-1-{4-[4-(trifluoromethyl)benzoyl]piperazin-1-yl}ethyl)phenoxy]-2,2-difluoroethanol;
3-(4-{(1R)-1-[4-(4-chlorobenzoyl)piperazin-1-yl]ethyl}-2,3-dimethylphenoxy)-N-isopropylpropan-1-amine;
3-[2,3-dimethyl-4-((1S)-1-{(1S,4S)-5-[4-(trifluoromethyl)benzoyl]-2,5-diazabicyclo[2.2.1]hept-2-yl}ethyl)phenoxy]propan-1-amine;
3-(4-{(1R)-1-[4-(4-chlorobenzoyl)piperazin-1-yl]ethyl}-2,3-dimethylphenoxy)-N,N-dimethylpropan-1-amine;
4-[2,3-dimethyl-4-((1R)-1-{4-[4-(trifluoromethyl)benzoyl]piperazin-1-yl}ethyl)phenoxy]butanenitrile;
4-[2,3-dimethyl-4-((1S)-1-{(1S,4S)-5-[4-(trifluoromethyl)benzoyl]-2,5-diazabicyclo[2.2.1]hept-2-yl}ethyl)phenoxy]butanenitrile;
4-(4-{(1R)-1-[4-(4-chlorobenzoyl)piperazin-1-yl]ethyl}-2,3-dimethylphenoxy)-2-methylbutan-2-ol;
N-[3-(4-{(1R)-1-[4-(4-chlorobenzoyl)piperazin-1-yl]ethyl}-2,3-dimethylphenoxy)-propyl]cyclopentanamine;
4-(4-{(1R)-1-[4-(4-chlorobenzoyl)piperazin-1-yl]ethyl}-2,3-dimethylphenoxy)-2-methylbutan-2-amine;
1-(1-{2,3-dimethyl-4-[2-(methylthio)ethoxy]phenyl}ethyl)-4-[4-(trifluoromethyl)-benzoyl]piperazine;
1-(4-chlorobenzoyl)-4-{(1R)-1-[4-(3-chloropropoxy)-2,3-dimethylphenyl]ethyl}piperazine;
3-[2,3-dimethyl-4-((1R)-1-{4-[4-(trifluoromethyl)benzoyl]piperazin-1-yl}ethyl)phenoxy]-N-methylpropan-1-amine;
3-(4-{(1R)-1-[4-(4-chlorobenzoyl)piperazin-1-yl]ethyl}-2,3-dimethylphenoxy)-N-methylpropan-1-amine;
4-(4-{(1R)-1-[4-(4-chlorobenzoyl)piperazin-1-yl]ethyl}-2,3-dimethyl-phenoxy)butanenitrile;
1-(1-{2,3-dimethyl-4-[2-(methylsulfonyl)ethoxy]phenyl}ethyl)-4-[4-(trifluoromethyl)-benzoyl]piperazine;
4-[2,3-dimethyl-4-((1R)-1-{4-[4-(trifluoromethyl)benzoyl]piperazin-1-yl}ethyl)phenoxy]-2-methylbutan-2-amine;
2-(4-{(1R)-1-[4-(4-chlorobenzoyl)piperazin-1-yl]ethyl}-2,3-dimethyl-phenoxy)acetamide;
3-(4-{(1R)-1-[4-(4-chlorobenzoyl)piperazin-1-yl]ethyl}-2,3-dimethylphenoxy)-propan-1-amine;
4-[2,3-dimethyl-4-((1R)-1-{4-[4-(trifluoromethyl)benzoyl]piperazin-1-yl}ethyl)phenoxy]butan-1-amine;
N-{3-[2,3-dimethyl-4-((1R)-1-{4-[4-(trifluoromethyl)benzoyl]-piperazin-1-yl}ethyl)phenoxy]-1,1-dimethylpropyl}acetamide;
N-[3-(4-{(1R)-1-[4-(4-chlorobenzoyl)piperazin-1-yl]ethyl}-2,3-dimethyl-phenoxy)propyl]acetamide;
N-[3-(4-{(1R)-1-[4-(4-chlorobenzoyl)piperazin-1-yl]ethyl}-2,3-dimethylphenoxy)-1,1-dimethylpropyl]acetamide;
1-[4-(2-methoxyethoxy)-2,3-dimethylbenzyl]-4-[4-(trifluoromethyl)benzoyl]piperazine;
4-[2,3-dimethyl-4-((1S)-1-{(1S,4S)-5-[4-(trifluoromethyl)benzoyl]-2,5-diazabicyclo[2.2.1]hept-2-yl}ethyl)phenoxy]-N-methylbutan-1-amine;
4-[2,3-dimethyl-4-((1S)-1-{(1S,4S)-5-[4-(trifluoromethyl)benzoyl]-2,5-diazabicyclo[2.2.1]hept-2-yl}ethyl)phenoxy]-N,N-dimethylbutan-1-amine;
4-[2,3-dimethyl-4-((1S)-1-{(1S,4S)-5-[4-(trifluoromethyl)benzoyl]-2,5-diazabicyclo[2.2.1]hept-2-yl}ethyl)phenoxy]butan-1-amine;
3-[2,3-dimethyl-4-({4-[4-(trifluoromethyl)benzoyl]piperazin-1-yl}methyl)phenoxy]propan-1-amine;
2-[2,3-dimethyl-4-((1R)-1-{4-[4-(trifluoromethyl)benzoyl]piperazin-1-yl}ethyl)phenoxy]ethanamine;
2-[2,3-dimethyl-4-((1R)-1-{4-[4-(trifluoromethyl)benzoyl]piperazin-1-yl}ethyl)phenoxy]-N-methylethanamine;
2-[2,3-dimethyl-4-((1S)-1-{(1S,4S)-5-[4-(trifluoromethyl)benzoyl]-2,5-diazabicyclo[2.2.1]hept-2-yl}ethyl)phenoxy]ethanamine;
2-[2,3-dimethyl-4-((1S)-1-{(1S,4S)-5-[4-(trifluoromethyl)benzoyl]-2,5-diazabicyclo[2.2.1]hept-2-yl}ethyl)phenoxy]-N-methylethanamine;
2-[2,3-dimethyl-4-((1S)-1-{(1S,4S)-5-[4-(trifluoromethyl)benzoyl]-2,5-diazabicyclo[2.2.1]hept-2-yl}ethyl)phenoxy]-N,N-dimethylethanamine;
2-(2,3-dimethyl-4-{2-[4-(trifluoromethyl)benzoyl]octahydro-2H-pyrido[1,2-a]pyrazin-6-yl}phenoxy)-N,N-dimethylethanamine;
4-(2,3-dimethyl-4-{2-[4-(trifluoromethyl)benzoyl]octahydro-2H-pyrido[1,2-a]pyrazin-6-yl}phenoxy)-N,N-dimethylbutan-1-amine;
(2,3-dimethyl-4-{2-[4-(trifluoromethyl)benzoyl]octahydro-2H-pyrido[1,2-a]pyrazin-6-yl}phenoxy)acetonitrile;
6-[4-(2-methoxyethoxy)-2,3-dimethylphenyl]-2-[4-(trifluoromethyl)benzoyl]octahydro-2H-pyrido-1,2-a]pyrazine;
6-[4-(3-methoxypropoxy)-2,3-dimethylphenyl]-2-[4-(trifluoromethyl)benzoyl]octahydro-2H-pyrido[1,2-a]pyrazine;
5-(2,3-dimethyl-4-{(6R,9aS)-2-[4-(trifluoromethyl)benzoyl]octa-hydro-2H-pyrido[1,2-a]pyrazin-6-yl}phenoxy)pentanenitrile;
4-(2,3-dimethyl-4-{(6R,9aS)-2-[4-(trifluoromethyl)benzoyl]octahydro-2H-pyrido[1,2-a]pyrazin-6-yl}phenoxy)butanenitrile;
(2R)-1-[2,3-dimethyl-4-(1-{4-[4-(trifluoromethyl)benzoyl]piperazin-1-yl}ethyl)phenoxy]-3-[(2-hydroxyethyl)amino]propan-2-ol;
(3S)-4-[2,3-dimethyl-4-(1-{4-[4-(trifluoromethyl)benzoyl]piperazin-1-yl}ethyl)phenoxy]-3-hydroxybutanenitrile;
1-(2,3-dimethyl-4-{(6R,9aS)-2-[4-(trifluoromethyl)benzoyl]octa-hydro-2H-pyrido[1,2-a]pyrazin-6-yl}phenoxy)-2-methylpropan-2-ol;
2-[2,3-dimethyl-4-(1-{4-[4-(trifluoromethyl)benzoyl]piperazin-1-yl}ethyl)phenoxy]-N,N-dimethylacetamide;
2-(2,3-dimethyl-4-{(6R,9aS)-2-[4-(trifluoromethyl)benzoyl]octahydro-2H-pyrido[1,2-a]pyrazin-6-yl}phenoxy)-N-ethyl-N-methylacetamide;
(6R,9aS)-6-[4-(allyloxy)-2,3-dimethylphenyl]-2-[4-(trifluoro-methyl)benzoyl]octahydro-2H-pyrido[1,2-a]pyrazine;
3-(2,3-dimethyl-4-{(6R,9aS)-2-[4-(trifluoromethyl)benzoyl]octa-hydro-2H-pyrido[1,2-a]pyrazin-6-yl}phenoxy)propan-1-ol;
(1S,4S)-2-[(6-chloropyridin-3-yl)carbonyl]-5-{(1S)-1-[4-(2-methoxyethoxy)-2,3-dimethylphenyl]ethyl}-2,5-diazabicyclo[2.2.1]heptane;
(1S,4S)-2-{(1S)-1-[4-(2-methoxyethoxy)-2,3-dimethyl-phenyl]ethyl}-5-{[6-(trifluoro-methyl)pyridin-3-yl]carbonyl}-2,5-diazabicyclo[2.2. I]heptane;
(2S)-1-[(cyclopropylmethyl)-amino]-3-(2,3-dimethyl-4-{(6R,9aS)-2-[4-(trifluoromethyl)-benzoyl]octahydro-2H-pyrido[1,2-a]pyrazin-6-yl}phenoxy)propan-2-ol;
(2S)-1-(cyclopentylamino)-3-(2,3-dimethyl-4-{(6R,9aS)-2-[4-(trifluoromethyl)benzoyl]octahydro-2H-pyrido[1,2-a]pyrazin-6-yl}phenoxy)propan-2-ol;
(1S,4S)-2-[(5-ethylpyridin-2-yl)carbonyl]-5-{(1S)-1-[4-(2-methoxyethoxy)-2,3-dimethylphenyl]ethyl}-2,5-diazabicyclo[2.2.1]heptane;
2-[2,3-dimethyl-4-((1R)-1-{4-[4-(trifluoromethyl)benzoyl]piperazin-1-yl}ethyl)phenoxy]-N-ethylacetamide;
2-[2,3-dimethyl-4-((1R)-1-{4-[4-(trifluoromethyl)benzoyl]piperazin-1-yl}ethyl)phenoxy]acetamide;
(1S,4S)-2-{(1S)-1-[4-(2-methoxyethoxy)-2,3-dimethylphenyl]ethyl}-5-{[6-(methylthio)pyridin-3-yl]carbonyl}-2,5-diazabicyclo[2.2.1]heptane;
(1S,4S)-2-{(1S)-1-[4-(2-methoxyethoxy)-2,3-dimethylphenyl]ethyl}-5-[(6-methylpyridin-3-yl)carbonyl]-2,5-diazabicyclo[2.2.1]heptane;
(6R,9aS)-6-[4-(2-methoxyethoxy)-2,3-dimethylphenyl]-2-[4-(trifluoromethyl)benzoyl]octahydro-2H-pyrido[1,2-a]pyrazine;
2-[2,3-dimethyl-4-((1R)-1-{(1R,4R)-5-[4-(trifluoromethyl)benzoyl]-2,5-diazabicyclo[2.2.1]hept-2-yl}ethyl)phenoxy]-N-methylacetamide;
2-[2,3-dimethyl-4-((1R)-1-{(1R,4R)-5-[4-(trifluoromethyl)benzoyl]-2,5-diazabicyclo[2.2.1]hept-2-yl}ethyl)phenoxy]-N,N-dimethylacetamide;
(2S)-1-[2,3-dimethyl-4-((1R)-1-{4-[4-(trifluoromethyl)benzoyl]piperazin-1-yl}ethyl)phenoxy]-3-[(2-methoxyethyl)amino]propan-2-ol;
(2R)-1-amino-3-[2,3-dimethyl-4-(1-{4-[4-(trifluoromethyl)benzoyl]-piperazin-1-yl}ethyl)-phenoxy]propan-2-ol;
(3R)-4-(2,3-dimethyl-4-{(6R,9aS)-2-[4-(trifluoromethyl)benzoyl]octa-hydro-2H-pyrido[1,2-a]pyrazin-6-yl}phenoxy)-3-hydroxybutanenitrile;
(3S)-4-(2,3-dimethyl-4-{(6R,9aS)-2-[4-(trifluoromethyl)benzoyl]octa-hydro-2H-pyrido[1,2-a]pyrazin-6-yl}phenoxy)-3-hydroxybutanenitrile;
(2R)-1-(dimethylamino)-3-[2,3-dimethyl-4-(1-{4-[4-(trifluoromethyl)benzoyl]piperazin-1-yl}ethyl)phenoxy]propan-2-ol;
(1S,4S)-2-[(6-ethylpyridin-3-yl)carbonyl]-5-{(1S)-1-[4-(2-methoxyethoxy)-2,3-dimethylphenyl]ethyl}-2,5-diazabicyclo[2.2.1]heptane;
(1S,4S)-2-[(6-isobutylpyridin-3-yl)carbonyl]-5-{(1S)-1-[4-(2-methoxyethoxy)-2,3-dimethylphenyl]ethyl}-2,5-diazabicyclo[2.2.1]heptane;
2-[2,3-dimethyl-4-((1R)-1-{(1R,4R)-5-[4-(trifluoromethyl)benzoyl]-2,5-diazabicyclo[2.2.1]hept-2-yl}ethyl)phenoxy]acetamide;
{2,3-dimethyl-4-[(1S)-1-((1S,4S)-5-{[6-(trifluoromethyl)pyridin-3-yl]carbonyl}-2,5-diazabicyclo[2.2.1]hept-2-yl)ethyl]phenoxy}acetonitrile;
2-{2,3-dimethyl-4-[(1S)-1-((1S,4S)-5-{[6-(trifluoromethyl)pyridin-3-yl]carbonyl}-2,5-diazabicyclo[2.2.1]hept-2-yl)ethyl]phenoxy}acetamide;
(3S)-4-[2,3-dimethyl-4-((1S)-1-{(1S,4S)-5-[4-(trifluoromethyl)-benzoyl]-2,5-diazabicyclo-[2.2.1]hept-2-yl}ethyl)phenoxy]-3-hydroxybutanenitrile;
(2S)-1-amino-3-[2,3-dimethyl-4-((S)-1-{(1S,4S)-5-[4-(trifluoromethyl)benzoyl]-2,5-diazabicyclo[2.2.1]hept-2-yl}ethyl)phenoxy]propan-2-ol;
(2S)-1-[2,3-dimethyl-4-((1S)-1-{(1S,4S)-5-[4-(trifluoromethyl)-benzoyl]-2,5-diazabicyclo-[2.2.1]hept-2-yl}ethyl)phenoxy]-3-[(2-methoxyethyl)amino]propan-2-ol;
(3R)-4-[2,3-dimethyl-4-((1S)-1-{(1S,4S)-5-[4-(trifluoromethyl)-benzoyl]-2,5-diazabicyclo[2.2.1]-hept-2-yl}ethyl)phenoxy]-3-hydroxybutanenitrile;
(2R)-1-amino-3-[2,3-dimethyl-4-((1S)-1-{(1S,4S)-5-[4-(trifluoromethyl)benzoyl]-2,5-diazabicyclo[2.2.1]hept-2-yl}ethyl)phenoxy]propan-2-ol;
(2R)-1-[2,3-dimethyl-4-((1S)-1-{(1S,4S)-5-[4-(trifluoromethyl)-benzoyl]-2,5-diazabicyclo[2.2.1]-hept-2-yl}ethyl)phenoxy]-3-[(2-methoxyethyl)amino]propan-2-ol;
4-{2,3-dimethyl-4-[(1S)-1-((1S,4S)-5-{[6-(trifluoromethyl)pyridin-3-yl]carbonyl}-2,5-diazabicyclo-[2.2.1]hept-2-yl)ethyl]phenoxy}-butanenitrile;
2-(2,3-dimethyl-4-{(6R,9aS)-2-[4-(trifluoromethyl)benzoyl]octahydro-2H-pyrido[1,2-a]pyrazin-6-yl}phenoxy)acetamide;
2-(2,3-dimethyl-4-{(6R,9aS)-2-[4-(trifluoromethyl)benzoyl]octahydro-2H-pyrido[1,2-a]pyrazin-6-yl}phenoxy)-N-methylacetamide;
2-(2,3-dimethyl-4-{(6R,9aS)-2-[4-(trifluoromethyl)benzoyl]octahydro-2H-pyrido[1,2-a]pyrazin-6-yl}phenoxy)-N,N-dimethylacetamide;
2-{2,3-dimethyl-4-[(1S)-1-((1S,4S)-5-{[6-(trifluoromethyl)pyridin-3-yl]carbonyl}-2,5-diazabicyclo-[2.2.1]hept-2-yl)ethyl]phenoxy}-N-methylacetamide;
2-{2,3-dimethyl-4-[(1S)-1-((1S,4S)-5-{[6-(trifluoromethyl)pyridin-3-yl]carbonyl}-2,5-diazabicyclo-[2.2.1]hept-2-yl)ethyl]phenoxy}-N,N-dimethylacetamide;
N-(3-{2,3-dimethyl-4-[(1S)-1-((1S,4S)-5-{[6-(trifluoromethyl)-pyridin-3-yl]carbonyl}-2,5-diazabicyclo[2.2.1]hept-2-yl)ethyl]phenoxy}propyl)acetamide;
(6R,9aS)-6-[4-(2-methoxyethoxy)-2,3-dimethylphenyl]-2-{[6-(trifluoromethyl)pyridin-3-yl]carbonyl}octahydro-2H-pyrido[1,2-a]pyrazine;
(6R,9aS)-6-[4-(3-methoxypropoxy)-2,3-dimethylphenyl]-2-{[6-(trifluoromethyl)pyridin-3-yl]carbonyl}octahydro-2H-pyrido[1,2-a]pyrazine;
[2,3-dimethyl-4-((6R,9aS)-2-{[6-(trifluoromethyl)pyridin-3-yl]carbonyl}octahydro-2H-pyrido[1,2-a]pyrazin-6-yl)phenoxy]acetonitrile;
1-{2,3-dimethyl-4-[(1S)-1-((1S,4S)-5-{[6-(trifluoromethyl)pyridin-3-yl]carbonyl}-2,5-diazabicyclo-[2.2.1]hept-2-yl)ethyl]phenoxy}-2-methylpropan-2-ol;
2-{2,3-dimethyl-4-[(1S)-1-((1S,4S)-5-{[6-(trifluoromethyl)pyridin-3-yl]carbonyl}-2,5-diazabicyclo[2.2.1]hept-2-yl)ethyl]phenoxy}ethanamine;
N-(2-{2,3-dimethyl-4-[(1S)-1-((1S,4S)-5-{[6-(trifluoromethyl)-pyridin-3-yl]carbonyl}-2,5-diazabicyclo[2.2.1]hept-2-yl)ethyl]-phenoxy}ethyl)acetamide;
(2R)-1-amino-3-(2,3-dimethyl-4-{(6R,9aS)-2-[4-(trifluoromethyl)-benzoyl]octahydro-2H-pyrido[1,2-a]pyrazin-6-yl}phenoxy)propan-2-ol;
(2S)-3-{2,3-dimethyl-4-[(1S)-1-((1S,4S)-5-{[6-(trifluoromethyl)-pyridin-3-yl]carbonyl}-2,5-diazabicyclo[2.2.1]hept-2-yl)ethyl]-phenoxy}-2-methylpropan-1-ol;
(2R)-3-{2,3-dimethyl-4-[(1S)-1-((1S,4S)-5-{[6-(trifluoromethyl)-pyridin-3-yl]carbonyl}-2,5-diazabicyclo[2.2.1]hept-2-yl)ethyl]-phenoxy}-2-methylpropan-1-ol;
(1S,4S)-2-{(1S)-1-[4-(2-methoxyethoxy)-2,3-dimethyl-phenyl]ethyl}-5-{[5-(trifluoro-methyl)pyridin-2-yl]carbonyl}-2,5-diazabicyclo[2.2.1]heptane;
(1S,4S)-2-[4-(2-methoxyethoxy)-2,3-dimethylbenzyl]-5-[4-(trifluoromethyl)benzoyl]-2,5-diazabicyclo[2.2.1]heptane;
2-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]ethanol;
(6R,9aS)-6-[4-(2-methoxyethoxy)-2,3-dimethylphenyl]-2-{[5-(trifluoromethyl)pyridin-2-yl]carbonyl}octahydro-2H-pyrido[1,2-a]pyrazine;
(1S,4S)-2-[(2-ethyl-1,3-thiazol-4-yl)carbonyl]-5-{(1S)-1-[4-(2-methoxyethoxy)-2,3-dimethylphenyl]ethyl}-2,5-diazabicyclo[2.2.1]heptane;
(6R,9aS)-6-[4-(2-ethoxyethoxy)-2,3-dimethylphenyl]-2-{[6-(trifluoro-methyl)pyridin-3-yl]carbonyl}-octahydro-2H-pyrido[1,2-a]pyrazine;
5-[((1S,4S)-5-{(1S)-1-[4-(2-methoxyethoxy)-2,3-dimethylphenyl]ethyl}-2,5-diazabicyclo[2.2.1]hept-2-yl)carbonyl]-2,1,3-benzoxadiazole;
5-[((1S,4S)-5-{(1S)-1-[4-(2-methoxyethoxy)-2,3-dimethylphenyl]ethyl}-2,5-diazabicyclo[2.2.1]hept-2-yl)carbonyl]-2,1,3-benzothiadiazole;
(6R,9aS)-6-{4-[2-(2-methoxy-ethoxy)ethoxy]-2,3-dimethyl-phenyl}-2-{[6-(trifluoromethyl)-pyridin-3-yl]carbonyl}octahydro-2H-pyrido[1,2-a]pyrazine;
(1S,4S)-2-{(1S)-1-[4-(2-methoxyethoxy)-2,3-dimethylphenyl]ethyl}-5-[(5-methyl-2-thienyl)carbonyl]-2,5-diazabicyclo[2.2. I]heptane;
(1S,4S)-2-{(1S)-1-[4-(2-methoxyethoxy)-2,3-dimethylphenyl]ethyl}-5-(3-thienylcarbonyl)-2,5-diazabicyclo[2.2.1]heptane;
(1S,4S)-2-{(1S)-1-[4-(2-methoxyethoxy)-2,3-dimethylphenyl]ethyl}-5-[4-(1H-pyrazol-1-yl)benzoyl]-2,5-diazabicyclo[2.2.1]heptane;
(1S,4S)-2-{(1S)-1-[4-(2-methoxy-ethoxy)-2,3-dimethylphenyl]ethyl}-5-{[2-(trifluoromethyl)-1,3-thiazol-4-yl]carbonyl}-2,5-diazabicyclo[2.2.1]heptane;
(1S,4S)-2-[(2-chloro-1,3-thiazol-4-yl)carbonyl]-5-{(1S)-1-[4-(2-methoxyethoxy)-2,3-dimethylphenyl]ethyl}-2,5-diazabicyclo[2.2.1]heptane;
(1S,4S)-2-[(2-chloro-5-methyl-1,3-thiazol-4-yl)carbonyl]-5-{(1S)-1-[4-(2-methoxyethoxy)-2,3-dimethylphenyl]ethyl}-2,5-diazabicyclo[2.2.1]heptane;
3-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]propan-1-ol;
1-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]acetone;
N-[(2R)-3-(2,3-dimethyl-4-{(6R,9aS)-2-[4-(trifluoromethyl)-benzoyl]octahydro-2H-pyrido-[1,2-a]pyrazin-6-yl}phenoxy)-2-hydroxypropyl]acetamide;
3-[2,3-dimethyl-4-((6R,9aS)-2-{[6-(trifluoromethyl)pyridin-3-yl]carbonyl}octahydro-2H-pyrido[1,2-a]pyrazin-6-yl)phenoxy]propan-1-amine;
(2E)-1-[2,3-dimethyl-4-((6R,9aS)-2-{[6-(trifluoromethyl)pyridin-3-yl]carbonyl}octahydro-2H-pyrido[1,2-a]pyrazin-6-yl)phenoxy]acetone oxime;
N-{(2R)-3-[2,3-dimethyl-4-((1S)-1-{(1S,4S)-5-[4-(trifluoromethyl)-benzoyl]-2,5-diazabicyclo[2.2.1]-hept-2-yl}ethyl)phenoxy]-2-hydroxypropyl}acetamide;
2-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]acetamide;
2-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]-N-methylacetamide;
2-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]-N-ethylacetamide;
2-[2,3-dimethyl-4-((6R,9aS)-2-{[6-(trifluoromethyl)pyridin-3-yl]-carbonyl}octahydro-2H-pyrido[1,2-a]pyrazin-6-yl)phenoxy]-N,N-dimethylacetamide;
N-Acetyl-N-(3-{2,3-dimethyl-4-[2-(6-trifluoromethyl-pyridine-3-carbonyl)-octahydro-pyrido-[1,2-a]pyrazin-6-yl]-phenoxy}-propyl)-acetamide;
N-{3-[2,3-dimethyl-4-((6R,9aS)-2-{[6-(trifluoromethyl)pyridin-3-yl]-carbonyl}octahydro-2H-pyrido-[1,2-a]pyrazin-6-yl)-phenoxy]propyl}acetamide;
(6R,9aS)-6-[4-(methoxymethoxy)-2,3-dimethylphenyl]-2-{[6-(trifluoromethyl)pyridin-3-yl]-carbonyl}octahydro-2H-pyrido-[1,2-a]pyrazine;
(2R)-1-[2,3-dimethyl-4-((6R,9aS)-2-{[6-(trifluoromethyl)pyridin-3-yl]carbonyl}octahydro-2H-pyrido[1,2-a]pyrazin-6-yl)-phenoxy]propan-2-ol;
2-(4-{(6R,9aS)-2-[(5-chloro-2-thienyl)carbonyl]octahydro-2H-pyrido[1,2-a]pyrazin-6-yl}-2,3-dimethylphenoxy)ethanol;
1-[2,3-dimethyl-4-((6R,9aS)-2-{[6-(trifluoromethyl)pyridin-3-yl]carbonyl}octahydro-2H-pyrido[1,2-a]pyrazin-6-yl)phenoxy]-2-methylpropan-2-ol;
(6R,9aS)-6-[4-(difluoromethoxy)-2,3-dimethylphenyl]-2-{[6-(trifluoromethyl)pyridin-3-yl]-carbonyl}octahydro-2H-pyrido[1,2-a]pyrazine;
(2S)-1-[2,3-dimethyl-4-((6R,9aS)-2-{[6-(trifluoromethyl)pyridin-3-yl]carbonyl}octahydro-2H-pyrido[1,2-a]pyrazin-6-yl)phenoxy]propan-2-ol;
2-[2,3-dimethyl-4-((6R,9aS)-2-{[5-(trifluoromethyl)-2-thienyl]carbonyl}octahydro-2H-pyrido[1,2-a]pyrazin-6-yl)phenoxy]ethanol;
(2S)-1-[2,3-dimethyl-4-((6R,9aS)-2-{[6-(chloro)pyridin-3-yl]carbonyl}-octahydro-2H-pyrido[1,2-a]pyrazin-6-yl)phenoxy]propan-2-ol;
(2R)-1-[2,3-dimethyl-4-((6R,9aS)-2-{[6-(chloro)pyridin-3-yl]carbonyl}-octahydro-2H-pyrido[1,2-a]pyrazin-6-yl)phenoxy]propan-2-ol;
(6-chloropyridin-3-yl)((6R,9aS)-6-(4-(2-hydroxyethoxy)-2,3-dimethylphenyl)-hexahydro-1H-pyrido[1,2-a]pyrazin-2 (6H)-yl)methanone;
(6-Chloro-pyridin-3-yl)-{(6R,9aS)-6-[4-(2-hydroxy-2-methyl-propoxy)-2,3-dimethyl-phenyl]-octahydro-pyrido[1,2-a]pyrazin-2-yl}-methanone;
(6-chloropyridin-3-yl)((6R,9aS)-6-(4-(1,1-difluoro-2-hydroxyethoxy)-2,3-dimethylphenyl)-hexahydro-1H-pyrido[1,2-a]pyrazin-2 (6H)-yl)methanone;
{(6R,9aS)-6-[4-(2-Hydroxy-1-hydroxymethyl-ethoxy)-2,3-dimethylphenyl]-octahydro-pyrido-[1,2-a]pyrazin-2-yl}-(6-trifluoro-methyl-pyridin-3-yl)-methanone;
{(6R,9aS)-6-[4-(2-Hydroxy-1-hydroxymethyl-ethoxy)-2,3-dimethylphenyl]-octahydro-pyrido-[1,2-a]pyrazin-2-yl}-(6-chloro-pyridin-3-yl)-methanone;
3-[2,3-dimethyl-4-(1-{4-[4-(trifluoromethyl)benzoyl]piperazin-1-yl}ethyl)phenoxy]-N-propylpropan-1-amine;
N-{3-[2,3-dimethyl-4-(1-{4-[4-(trifluoromethyl)benzoyl]piperazin-1-yl}ethyl)phenoxy]propyl}-2-methylbutan-1-amine;
N-(cyclopropylmethyl)-3-[2,3-dimethyl-4-(1-{4-[4-(trifluoromethyl)benzoyl]piperazin-1-yl}ethyl)phenoxy]propan-1-amine;
N-{3-[2,3-dimethyl-4-(1-{4-[4-(trifluoromethyl)benzoyl]piperazin-1-yl}ethyl)phenoxy]propyl}-2-methylpropan-1-amine;
N-(cyclohexylmethyl)-3-[2,3-dimethyl-4-(1-{4-[4-(trifluoromethyl)benzoyl]piperazin-1-yl}ethyl)phenoxy]propan-1-amine;
N-{3-[2,3-dimethyl-4-(1-{4-[4-(trifluoromethyl)benzoyl]piperazin-1-yl}ethyl)phenoxy]propyl}-2,2-dimethylpropan-1-amine;
3-[2,3-dimethyl-4-(1-{4-[4-(trifluoromethyl)benzoyl]piperazin-1-yl}ethyl)phenoxy]-N-(2-ethoxyethyl)propan-1-amine;
3-[2,3-dimethyl-4-(1-{4-[4-(trifluoromethyl)benzoyl]piperazin-1-yl}ethyl)phenoxy]-N-(2-isopropoxyethyl)propan-1-amine;
N′-{3-[2,3-dimethyl-4-(1-{4-[4-(trifluoromethyl)benzoyl]piperazin-1-yl}ethyl)phenoxy]propyl}-N,N-dimethylethane-1,2-diamine;
N-{3-[2,3-dimethyl-4-(1-{4-[4-(trifluoromethyl)benzoyl]piperazin-1-yl}ethyl)phenoxy]propyl}-cyclopropanamine;
N-{3-[2,3-dimethyl-4-(1-{4-[4-(trifluoromethyl)benzoyl]piperazin-1-yl}ethyl)phenoxy]propyl}-cyclohexanamine;
N-{3-[2,3-dimethyl-4-(1-{4-[4-(trifluoromethyl)benzoyl]piperazin-1-yl}ethyl)phenoxy]propyl}cyclobutanamine;
N-{3-[2,3-dimethyl-4-(1-{4-[4-(trifluoromethyl)benzoyl]piperazin-1-yl}ethyl)phenoxy]propyl}cyclopentanamine;
N-{3-[2,3-dimethyl-4-(1-{4-[4-(trifluoromethyl)benzoyl]piperazin-1-yl}ethyl)phenoxy]propyl}-4-methylcyclohexanamine;
N-{3-[2,3-dimethyl-4-(1-{4-[4-(trifluoromethyl)benzoyl]piperazin-1-yl}ethyl)phenoxy]propyl}-2-methylcyclohexanamine;
3-[2,3-dimethyl-4-(1-{4-[4-(trifluoromethyl)benzoyl]piperazin-1-yl}ethyl)phenoxy]-N-isopropylpropan-1-amine;
N-{3-[2,3-dimethyl-4-(1-{4-[4-(trifluoromethyl)benzoyl]piperazin-1-yl}ethyl)phenoxy]propyl}butan-2-amine;
N-{3-[2,3-dimethyl-4-(1-{4-[4-(trifluoromethyl)benzoyl]piperazin-1-yl}ethyl)phenoxy]propyl}-3-methylbutan-2-amine;
3-[2,3-dimethyl-4-(1-{4-[4-(trifluoromethyl)benzoyl]piperazin-1-yl}ethyl)phenoxy]-N-(2-methoxy-1-methylethyl)propan-1-amine;
N-(tert-butyl)-3-[2,3-dimethyl-4-(1-{4-[4-(trifluoromethyl)benzoyl]-piperazin-1-yl}ethyl)phenoxy]-propan-1-amine;
N²-{3-[2,3-dimethyl-4-(1-{4-[4-(trifluoromethyl)benzoyl]piperazin-1-yl}ethyl)phenoxy]propyl}-N′,N′-dimethylpropane-1,2-diamine;
3-[2,3-dimethyl-4-(1-{4-[4-(trifluoromethyl)benzoyl]piperazin-1-yl}ethyl)phenoxy]-N,N-diethylpropan-1-amine;
3-[2,3-dimethyl-4-(1-{4-[4-(trifluoromethyl)benzoyl]piperazin-1-yl}ethyl)phenoxy]-N-methyl-N-propylpropan-1-amine;
N-{3-[2,3-dimethyl-4-(1-{4-[4-(trifluoromethyl)benzoyl]piperazin-1-yl}ethyl)phenoxy]propyl}-N-methylbutan-1-amine;
3-[2,3-dimethyl-4-(1-{4-[4-(trifluoromethyl)benzoyl]piperazin-1-yl}ethyl)phenoxy]-N-ethyl-N-isopropylpropan-1-amine;
3-[2,3-dimethyl-4-(1-{4-[4-(trifluoromethyl)benzoyl]piperazin-1-yl}ethyl)phenoxy]-N,N-dipropylpropan-1-amine;
3-[2,3-dimethyl-4-(1-{4-[4-(trifluoromethyl)benzoyl]piperazin-1-yl}ethyl)phenoxy]-N,N-dimethylpropan-1-amine;
3-[2,3-dimethyl-4-(1-{4-[4-(trifluoromethyl)benzoyl]piperazin-1-yl}ethyl)phenoxy]-N-isopropyl-N-methylpropan-1-amine;
N-{3-[2,3-dimethyl-4-(1-{4-[4-(trifluoromethyl)benzoyl]piperazin-1-yl}ethyl)phenoxy]propyl}-N-methylcyclohexanamine;
N-{3-[2,3-dimethyl-4-(1-{4-[4-(trifluoromethyl)benzoyl]piperazin-1-yl}ethyl)phenoxy]propyl}-N-ethylcyclohexanamine;
3-[2,3-dimethyl-4-(1-{4-[4-(trifluoromethyl)benzoyl]piperazin-1-yl}ethyl)phenoxy]-N,N-diisopropylpropan-1-amine;
N-{2-[2,3-dimethyl-4-(1-{4-[4-(trifluoromethyl)benzoyl]piperazin-1-yl}ethyl)phenoxy]ethyl}propan-1-amine;
N-{2-[2,3-dimethyl-4-(1-{4-[4-(trifluoromethyl)benzoyl]piperazin-1-yl}ethyl)phenoxy]ethyl}-2-methylbutan-1-amine;
N-(cyclopropylmethyl)-2-[2,3-dimethyl-4-(1-{4-[4-(trifluoromethyl)benzoyl]piperazin-1-yl}ethyl)phenoxy]ethanamine;
N-{2-[2,3-dimethyl-4-(1-{4-[4-(trifluoromethyl)benzoyl]piperazin-1-yl}ethyl)phenoxy]ethyl}-2-methylpropan-1-amine;
N-(cyclohexylmethyl)-2-[2,3-dimethyl-4-(1-{4-[4-(trifluoromethyl)benzoyl]piperazin-1-yl}ethyl)phenoxy]ethanamine;
N-{2-[2,3-dimethyl-4-(1-{4-[4-(trifluoromethyl)benzoyl]piperazin-1-yl}ethyl)phenoxy]ethyl}-2,2-dimethylpropan-1-amine;
2-[2,3-dimethyl-4-(1-{4-[4-(trifluoromethyl)benzoyl]piperazin-1-yl}ethyl)phenoxy]-N-(2-ethoxyethyl)ethanamine;
2-[2,3-dimethyl-4-(1-{4-[4-(trifluoromethyl)benzoyl]piperazin-1-yl}ethyl)phenoxy]-N-(2-isopropoxyethyl)ethanamine;
N′-{2-[2,3-dimethyl-4-(1-{4-[4-(trifluoromethyl)benzoyl]piperazin-1-yl}ethyl)phenoxy]ethyl}-N,N-dimethylethane-1,2-diamine;
N-{2-[2,3-dimethyl-4-(1-{4-[4-(trifluoromethyl)benzoyl]piperazin-1-yl}ethyl)phenoxy]ethyl}-cyclopropanamine;
N-{2-[2,3-dimethyl-4-(1-{4-[4-(trifluoromethyl)benzoyl]piperazin-1-yl}ethyl)phenoxy]ethyl}cyclohexanamine;
N-{2-[2,3-dimethyl-4-(1-{4-[4-(trifluoromethyl)benzoyl]piperazin-1-yl}ethyl)phenoxy]ethyl}cyclobutanamine;
N-{2-[2,3-dimethyl-4-(1-{4-[4-(trifluoromethyl)benzoyl]piperazin-1-yl}ethyl)phenoxy]ethyl}cyclopentanamine;
N-{2-[2,3-dimethyl-4-(1-{4-[4-(trifluoromethyl)benzoyl]piperazin-1-yl}ethyl)phenoxy]ethyl}-4-methylcyclohexanamine;
N-{2-[2,3-dimethyl-4-(1-{4-[4-(trifluoromethyl)benzoyl]piperazin-1-yl}ethyl)phenoxy]ethyl}-2-methylcyclohexanamine;
N-{2-[2,3-dimethyl-4-(1-{4-[4-(trifluoromethyl)benzoyl]piperazin-1-yl}ethyl)phenoxy]ethyl}propan-2-amine;
N-{2-[2,3-dimethyl-4-(1-{4-[4-(trifluoromethyl)benzoyl]piperazin-1-yl}ethyl)phenoxy]ethyl}butan-2-amine;
N-{2-[2,3-dimethyl-4-(1-{4-[4-(trifluoromethyl)benzoyl]piperazin-1-yl}ethyl)phenoxy]ethyl}-3-methylbutan-2-amine;
N-{2-[2,3-dimethyl-4-(1-{4-[4-(trifluoromethyl)benzoyl]piperazin-1-yl}ethyl)phenoxy]ethyl}-1-methoxypropan-2-amine;
N-{2-[2,3-dimethyl-4-(1-{4-[4-(trifluoromethyl)benzoyl]piperazin-1-yl}ethyl)phenoxy]ethyl}-2-methylpropan-2-amine;
N²-{2-[2,3-dimethyl-4-(1-{4-[4-(trifluoromethyl)benzoyl]piperazin-1-yl}ethyl)phenoxy]ethyl}-N¹,N¹-dimethylpropane-1,2-diamine;
2-[2,3-dimethyl-4-(1-{4-[4-(trifluoromethyl)benzoyl]piperazin-1-yl}ethyl)phenoxy]-N,N-diethylethanamine;
N-{2-[2,3-dimethyl-4-(1-{4-[4-(trifluoromethyl)benzoyl]piperazin-1-yl}ethyl)phenoxy]ethyl}-N-methylpropan-1-amine;
N-{2-[2,3-dimethyl-4-(1-{4-[4-(trifluoromethyl)benzoyl]piperazin-1-yl}ethyl)phenoxy]ethyl}-N-methylbutan-1-amine;
N-{2-[2,3-dimethyl-4-(1-{4-[4-(trifluoromethyl)benzoyl]piperazin-1-yl}ethyl)phenoxy]ethyl}-N-ethylpropan-2-amine;
N-{2-[2,3-dimethyl-4-(1-{4-[4-(trifluoromethyl)benzoyl]piperazin-1-yl}ethyl)phenoxy]ethyl}-N-propylpropan-1-amine;
2-[2,3-dimethyl-4-(1-{4-[4-(trifluoromethyl)benzoyl]piperazin-1-yl}ethyl)phenoxy]-N,N-dimethylethanamine;
N-{2-[2,3-dimethyl-4-(1-{4-[4-(trifluoromethyl)benzoyl]piperazin-1-yl}ethyl)phenoxy]ethyl}-N-methylpropan-2-amine;
N-{2-[2,3-dimethyl-4-(1-{4-[4-(trifluoromethyl)benzoyl]piperazin-1-yl}ethyl)phenoxy]ethyl}-N-methylcyclohexanamine;
N-{2-[2,3-dimethyl-4-(1-{4-[4-(trifluoromethyl)benzoyl]piperazin-1-yl}ethyl)phenoxy]ethyl}-N-ethylcyclohexanamine;
N-{2-[2,3-dimethyl-4-(1-{4-[4-(trifluoromethyl)benzoyl]piperazin-1-yl}ethyl)phenoxy]ethyl}-N-isopropylpropan-2-amine;
4-[2,3-dimethyl-4-(1-{4-[4-(trifluoromethyl)benzoyl]piperazin-1-yl}ethyl)phenoxy]-N-propylbutan-1-amine;
N-{4-[2,3-dimethyl-4-(1-{4-[4-(trifluoromethyl)benzoyl]piperazin-1-yl}ethyl)phenoxy]butyl}-2-methylbutan-1-amine;
N-(cyclopropylmethyl)-4-[2,3-dimethyl-4-(1-{4-[4-(trifluoromethyl)benzoyl]piperazin-1-yl}ethyl)phenoxy]butan-1-amine;
4-[2,3-dimethyl-4-(1-{4-[4-(trifluoromethyl)benzoyl]piperazin-1-yl}ethyl)phenoxy]-N-isobutylbutan-1-amine;
N-(cyclohexylmethyl)-4-[2,3-dimethyl-4-(1-{4-[4-(trifluoromethyl)benzoyl]piperazin-1-yl}ethyl)phenoxy]butan-1-amine;
N-(2,2-dimethylpropyl)-4-[2,3-dimethyl-4-(1-{4-[4-(trifluoromethyl)benzoyl]piperazin-1-yl}ethyl)phenoxy]butan-1-amine;
4-[2,3-dimethyl-4-(1-{4-[4-(trifluoromethyl)benzoyl]piperazin-1-yl}ethyl)phenoxy]-N-(2-ethoxyethyl)butan-1-amine;
4-[2,3-dimethyl-4-(1-{4-[4-(trifluoromethyl)benzoyl]piperazin-1-yl}ethyl)phenoxy]-N-(2-isopropoxyethyl)butan-1-amine;
N′-{4-[2,3-dimethyl-4-(1-{4-[4-(trifluoromethyl)benzoyl]piperazin-1-yl}ethyl)phenoxy]butyl}-N,N-dimethylethane-1,2-diamine;
N-{4-[2,3-dimethyl-4-(1-{4-[4-(trifluoromethyl)benzoyl]piperazin-1-yl}ethyl)phenoxy]butyl}cyclopropanamine;
N-{4-[2,3-dimethyl-4-(1-{4-[4-(trifluoromethyl)benzoyl]piperazin-1-yl}ethyl)phenoxy]butyl}cyclohexanamine;
N-{4-[2,3-dimethyl-4-(1-{4-[4-(trifluoromethyl)benzoyl]piperazin-1-yl}ethyl)phenoxy]butyl}cyclobutanamine;
N-{4-[2,3-dimethyl-4-(1-{4-[4-(trifluoromethyl)benzoyl]piperazin-1-yl}ethyl)phenoxy]butyl}cyclopentanamine;
N-{4-[2,3-dimethyl-4-(1-{4-[4-(trifluoromethyl)benzoyl]piperazin-1-yl}ethyl)phenoxy]butyl}-4-methylcyclohexanamine;
N-{4-[2,3-dimethyl-4-(1-{4-[4-(trifluoromethyl)benzoyl]piperazin-1-yl}ethyl)phenoxy]butyl}-2-methylcyclohexanamine;
4-[2,3-dimethyl-4-(1-{4-[4-(trifluoromethyl)benzoyl]piperazin-1-yl}ethyl)phenoxy]-N-isopropylbutan-1-amine;
N-(sec-butyl)-4-[2,3-dimethyl-4-(1-{4-[4-(trifluoromethyl)benzoyl]piperazin-1-yl}ethyl)phenoxy]butan-1-amine;
N-(1,2-dimethylpropyl)-4-[2,3-dimethyl-4-(1-{4-[4-(trifluoromethyl)benzoyl]piperazin-1-yl}ethyl)phenoxy]butan-1-amine;
4-[2,3-dimethyl-4-(1-{4-[4-(trifluoromethyl)benzoyl]piperazin-1-yl}ethyl)phenoxy]-N-(2-methoxy-1-methylethyl)butan-1-amine;
N-(tert-butyl)-4-[2,3-dimethyl-4-(1-{4-[4-(trifluoromethyl)benzoyl]piperazin-1-yl}ethyl)phenoxy]butan-1-amine;
N²-{4-[2,3-dimethyl-4-(1-{4-[4-(trifluoromethyl)benzoyl]piperazin-1-yl}ethyl)phenoxy]butyl}-N′,N′-dimethylpropane-1,2-diamine;
4-[2,3-dimethyl-4-(1-{4-[4-(trifluoromethyl)benzoyl]piperazin-1-yl}ethyl)phenoxy]-N,N-diethylbutan-1-amine;
4-[2,3-dimethyl-4-(1-{4-[4-(trifluoromethyl)benzoyl]piperazin-1-yl}ethyl)phenoxy]-N-methyl-N-propylbutan-1-amine;
N-butyl-4-[2,3-dimethyl-4-(1-{4-[4-(trifluoromethyl)benzoyl]piperazin-1-yl}ethyl)phenoxy]-N-methylbutan-1-amine;
4-[2,3-dimethyl-4-(1-{4-[4-(trifluoromethyl)benzoyl]piperazin-1-yl}ethyl)phenoxy]-N-ethyl-N-isopropylbutan-1-amine;
4-[2,3-dimethyl-4-(1-{4-[4-(trifluoromethyl)benzoyl]piperazin-1-yl}ethyl)phenoxy]-N,N-dipropylbutan-1-amine;
4-[2,3-dimethyl-4-(1-{4-[4-(trifluoromethyl)benzoyl]piperazin-1-yl}ethyl)phenoxy]-N,N-dimethylbutan-1-amine;
4-[2,3-dimethyl-4-(1-{4-[4-(trifluoromethyl)benzoyl]piperazin-1-yl}ethyl)phenoxy]-N-isopropyl-N-methylbutan-1-amine;
N-{4-[2,3-dimethyl-4-(1-{4-[4-(trifluoromethyl)benzoyl]piperazin-1-yl}ethyl)phenoxy]butyl}-N-methylcyclohexanamine;
N-{4-[2,3-dimethyl-4-(1-{4-[4-(trifluoromethyl)benzoyl]piperazin-1-yl}ethyl)phenoxy]butyl}-N-ethylcyclohexanamine;
4-[2,3-dimethyl-4-(1-{4-[4-(trifluoromethyl)benzoyl]piperazin-1-yl}ethyl)phenoxy]-N,N-diisopropylbutan-1-amine;
(6R,9aS)-2-(4-chloro-3-methoxyphenyl)-6-[4-(2-methoxyethoxy)-2,3-dimethylphenyl]octahydro-2H-pyrido[1,2-a]pyrazine;
3-{4-[(6R,9aS)-2-(4-chloro-3-methoxyphenyl)octahydro-2H-pyrido[1,2-a]pyrazin-6-yl]-2,3-dimethylphenoxy}-N-ethylpropan-1-amine;
3-{4-[(6R,9aS)-2-(4-chloro-3-methoxyphenyl)octahydro-2H-pyrido[1,2-a]pyrazin-6-yl]-2,3-dimethylphenoxy}-N,N-dimethylpropan-1-amine;
3-{4-[(6R,9aS)-2-(4-chloro-3-methoxyphenyl)octahydro-2H-pyrido[1,2-a]pyrazin-6-yl]-2,3-dimethylphenoxy}-N-isopropylpropan-1-amine;
(6R,9aS)-2-(4-chloro-3-methoxyphenyl)-6-[4-(3-methoxypropoxy)-2,3-dimethylphenyl]octahydro-2H-pyrido[1,2-a]pyrazine;
(6R,9aS)-2-(4-chloro-3-methoxyphenyl)-6-{4-[2-ethoxy-1-(ethoxymethyl)ethoxy]-2,3-dimethylphenyl}octahydro-2H-pyrido[1,2-a]pyrazine;
4-{4-[(6R,9aS)-2-(4-chloro-3-methoxyphenyl)octahydro-2H-pyrido[1,2-a]pyrazin-6-yl]-2,3-dimethylphenoxy}butanenitrile;
(6R,9aS)-2-(4-chloro-3-methoxyphenyl)-6-{2,3-dimethyl-4-[3-(methylsulfonyl)propoxy]-phenyl}octahydro-2H-pyrido-[1,2-a]pyrazine;
2-{4-[(6R,9aS)-2-(4-chloro-3-methoxyphenyl)octahydro-2H-pyrido[1,2-a]pyrazin-6-yl]-2,3-dimethylphenoxy}acetamide;
4-{4-[(6R,9aS)-2-(4-chloro-3-methoxyphenyl)octahydro-2H-pyrido[1,2-a]pyrazin-6-yl]-2-methoxyphenoxy}butanenitrile;
3-{4-[(6R,9aS)-2-(4-chloro-3-methoxyphenyl)octahydro-2H-pyrido[1,2-a]pyrazin-6-yl]-2-methoxyphenoxy}-N,N-dimethylpropan-1-amine;
3-{4-[(6R,9aS)-2-(4-chloro-3-methoxyphenyl)octahydro-2H-pyrido[1,2-a]pyrazin-6-yl]-2-methoxyphenoxy}-N-isopropylpropan-1-amine;
3-{4-[(6R,9aS)-2-(4-chloro-3-methoxyphenyl)octahydro-2H-pyrido[1,2-a]pyrazin-6-yl]-2-methoxyphenoxy}-N-methylpropan-1-amine;
(6R,9aS)-2-(4-chloro-3-methoxyphenyl)-6-[4-(2-methoxyethoxy)-2,3-dimethylphenyl]octahydro-8H-pyrido[1,2-a]pyrazin-8-one;
(6R,9aS)-2-(4-chloro-3-methoxyphenyl)-6-[3-methoxy-4-(2-methoxyethoxy)phenyl]octa-hydro-2H-pyrido[1,2-a]pyrazine;
3-{4-[(6R,9aS)-2-(4-chloro-3-methoxyphenyl)octahydro-2H-pyrido[1,2-a]pyrazin-6-yl]-2,3-dimethylphenoxy}-N,N-bis(2-methoxyethyl)propan-1-amine;
3-{4-[(6R,9aS)-2-(4-chloro-3-methoxyphenyl)octahydro-2H-pyrido[1,2-a]pyrazin-6-yl]-2,3-dimethylphenoxy}-N-(2-methoxyethyl)propan-1-amine;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-N-(2-methoxyethyl)acetamide;
methyl {4-[(6R,9aS)-2-(4-chloro-3-methoxyphenyl)octahydro-2H-pyrido[1,2-a]pyrazin-6-yl]-2,3-dimethylphenoxy}acetate;
1-{4-[(6R,9aS)-2-(4-chloro-3-methoxyphenyl)octahydro-2H-pyrido[1,2-a]pyrazin-6-yl]-2,3-dimethylphenoxy}-2-methylpropan-2-ol;
(6R,9aS)-2-(4-chloro-3-methoxyphenyl)-6-(4-isobutoxy-2,3-dimethylphenyl)octahydro-2H-pyrido[1,2-a]pyrazine;
2-{4-[(6R,9aS)-2-(4-chloro-3-methoxyphenyl)octahydro-2H-pyrido[1,2-a]pyrazin-6-yl]-2,3-dimethylphenoxy}-N-(2-methoxyethyl)acetamide;
(6R,9aS)-2-(4-chloro-3-methoxyphenyl)-6-{4-[2-(2-methoxyethoxy)ethoxy]-2,3-dimethylphenyl}octahydro-2H-pyrido[1,2-a]pyrazine;
2-{4-[(6R,9aS)-2-(4-chloro-3-methoxyphenyl)octahydro-2H-pyrido[1,2-a]pyrazin-6-yl]-2,3-dimethylphenoxy}-N-methylacetamide;
2-{4-[(6R,9aS)-2-(4-chloro-3-methoxyphenyl)octahydro-2H-pyrido[1,2-a]pyrazin-6-yl]-2,3-dimethylphenoxy}-N,N-dimethylacetamide;
{4-[(6R,9aS)-2-(4-chloro-3-methoxyphenyl)octahydro-2H-pyrido[1,2-a]pyrazin-6-yl]-2,3-dimethylphenoxy}acetonitrile;
2-{4-[(6R,9aS)-2-(4-chloro-3-methoxyphenyl)octahydro-2H-pyrido[1,2-a]pyrazin-6-yl]-2,3-dimethylphenoxy}ethanimidamide;
(6R,9aS)-2-(4-chloro-3-methoxyphenyl)-6-[4-(cyclopropylmethoxy)-2,3-dimethylphenyl]octahydro-2H-pyrido[1,2-a]pyrazine;
3-{4-[(6R,9aS)-2-(4-chloro-3-methoxyphenyl)octahydro-2H-pyrido[1,2-a]pyrazin-6-yl]-2,3-dimethylphenoxy}-N-methoxy-N-methylpropan-1-amine;
3-{4-[(6R,9aS)-2-(4-chloro-3-methoxyphenyl)octahydro-2H-pyrido[1,2-a]pyrazin-6-yl]-2,3-dimethylphenoxy}-N-methoxypropan-1-amine;
4-{4-[(6R,9aS)-2-(4-chloro-3-methoxyphenyl)octahydro-2H-pyrido[1,2-a]pyrazin-6-yl]-2,3-dimethylphenoxy}-N,N-dimethylbutanamide;
2-{4-[(6R,9aS)-2-(4-chloro-3-methoxyphenyl)octahydro-2H-pyrido[1,2-a]pyrazin-6-yl]-2,3-dimethylphenoxy}-N-ethyl-N-methylacetamide;
2-{4-[(6R,9aS)-2-(4-chloro-3-methoxyphenyl)octahydro-2H-pyrido[1,2-a]pyrazin-6-yl]-2,3-dimethylphenoxy}-N-methyl-N-propylacetamide;
N-butyl-2-{4-[(6R,9aS)-2-(4-chloro-3-methoxyphenyl)octahydro-2H-pyrido[1,2-a]pyrazin-6-yl]-2,3-dimethylphenoxy}-N-methylacetamide;
2-{4-[(6R,9aS)-2-(4-chloro-3-methoxyphenyl)octahydro-2H-pyrido[1,2-a]pyrazin-6-yl]-2,3-dimethylphenoxy}propanenitrile;
(6R,9aS)-2-(4-chloro-3-methoxyphenyl)-6-[4-(2-isopropoxyethoxy)-2,3-dimethylphenyl]octahydro-2H-pyrido[1,2-a]pyrazine;
(6R,9aS)-2-(4-chloro-3-methoxyphenyl)-6-(3-methoxy-4-propoxyphenyl)octahydro-2H-pyrido[1,2-a]pyrazine;
4-{4-[(6R,9aS)-2-(4-chloro-3-methoxyphenyl)octahydro-2H-pyrido[1,2-a]pyrazin-6-yl]-2,3-dimethylphenoxy}butanamide;
4-{4-[(6R,9aS)-2-(4-chloro-3-methoxyphenyl)octahydro-2H-pyrido[1,2-a]pyrazin-6-yl]-2,3-dimethylphenoxy}-N-methylbutanamide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-2-methoxyacetamide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)methane-sulfonamide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)acetamide;
2-{4-[(6R,9aS)-2-(4-chloro-3-methoxyphenyl)octahydro-2H-pyrido[1,2-a]pyrazin-6-yl]-2,3-dimethylphenoxy}-N-methoxyacetamide;
2-{4-[(6R,9aS)-2-(4-chloro-3-methoxyphenyl)octahydro-2H-pyrido[1,2-a]pyrazin-6-yl]-2,3-dimethylphenoxy}-N-[2-(dimethylamino)ethyl]acetamide;
{4-[(6R,9aS)-2-(4-chloro-3-methoxyphenyl)octahydro-2H-pyrido[1,2-a]pyrazin-6-yl]-2,3-dimethylphenoxy}acetic acid;
2-{4-[(6R,9aS)-2-(4-chloro-3-methoxyphenyl)octahydro-2H-pyrido[1,2-a]pyrazin-6-yl]-2,3-dimethylphenoxy}-N-ethylacetamide;
1-{4-[(6R,9aS)-2-(4-chloro-3-methoxyphenyl)octahydro-2H-pyrido[1,2-a]pyrazin-6-yl]-2,3-dimethylphenoxy}-3-[(2-methoxyethyl)amino]propan-2-ol;
1-{4-[(6R,9aS)-2-(4-chloro-3-methoxyphenyl)octahydro-2H-pyrido[1,2-a]pyrazin-6-yl]-2,3-dimethylphenoxy}-3-[(2-methoxyethyl)(methyl)amino]propan-2-ol;
1-{4-[(6R,9aS)-2-(4-chloro-3-methoxyphenyl)octahydro-2H-pyrido[1,2-a]pyrazin-6-yl]-2,3-dimethylphenoxy}-3-(cyclopropylamino)propan-2-ol;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-propanamide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-2-methylpropanamide;
N-(3-{4-[(6R,9aS)-2-(4-Chloro-3-methoxy-phenyl)-octahydro-pyrido[1,2-a]pyrazin-6-yl]-2,3-dimethylphenoxy}-propyl)-N-isobutyryl-isobutyramide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-2,2-dimethylpropanamide;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-formamide;
3-{4-[2-(4-chloro-3-methoxyphenyl)octahydro-2H-pyrido[1,2-a]pyrazin-6-yl]-2,3-dimethylphenoxy}propan-1-ol;
N-(2-{4-[(6R,9aS)-2-(4-chloro-3-methoxyphenyl)octahydro-2H-pyrido[1,2-a]pyrazin-6-yl]-2,3-dimethylphenoxy}ethyl)acetamide;
(6R,9aS)-2-(4-fluoro-3-methoxy-phenyl)-6-[4-(2-methoxyethoxy)-2,3-dimethylphenyl]octahydro-2H-pyrido[1,2-a]pyrazine;
N-(3-{4-[(6R,9aS)-2-(4-fluoro-3-methoxyphenyl)octahydro-2H-pyrido[1,2-a]pyrazin-6-yl]-2,3-dimethylphenoxy}propyl)acetamide;
2-{4-[(6R,9aS)-2-(4-fluoro-3-methoxyphenyl)octahydro-2H-pyrido[1,2-a]pyrazin-6-yl]-2,3-dimethylphenoxy}-N,N-dimethylacetamide;
1-{4-[(6R,9aS)-2-(4-chloro-3-methoxyphenyl)octahydro-2H-pyrido[1,2-a]pyrazin-6-yl]-2,3-dimethylphenoxy}acetone;
3-{4-[(6R,9aS)-2-(4-chloro-3-methoxyphenyl)octahydro-2H-pyrido[1,2-a]pyrazin-6-yl]-2,3-dimethylphenoxy}-2,2-dimethylpropan-1-ol;
(6R,9aS)-2-(4-fluoro-3-methoxyphenyl)-6-[4-(2-methoxyethoxy)-2,3-dimethylphenyl]octahydro-2H-pyrido[1,2-a]pyrazine;
1-{4-[(6R,9aS)-2-(4-chloro-3-methoxyphenyl)octahydro-2H-pyrido[1,2-a]pyrazin-6-yl]-2,3-dimethylphenoxy}-3,3-dimethylbutan-2-one;
2-{4-[(6R,9aS)-2-(4-fluoro-3-methoxyphenyl)octahydro-2H-pyrido[1,2-a]pyrazin-6-yl]-2,3-dimethylphenoxy}-N,N-dimethylacetamide;
N-ethyl-2-{4-[(6R,9aS)-2-(4-fluoro-3-methoxyphenyl)octahydro-2H-pyrido[1,2-a]pyrazin-6-yl]-2,3-dimethylphenoxy}acetamide;
2-{4-[(6R,9aS)-2-(4-fluoro-3-methoxyphenyl)octahydro-2H-pyrido[1,2-a]pyrazin-6-yl]-2,3-dimethylphenoxy}-N-methylacetamide;
2-{4-[(6R,9aS)-2-(4-fluoro-3-methoxyphenyl)octahydro-2H-pyrido[1,2-a]pyrazin-6-yl]-2,3-dimethylphenoxy}acetamide
3-{4-[(6R,9aS)-2-(4-fluoro-3-methoxyphenyl)octahydro-2H-pyrido[1,2-a]pyrazin-6-yl]-2,3-dimethylphenoxy}propan-1-ol;
3-{4-[(6R,9aS)-2-(4-chloro-3-methoxyphenyl)octahydro-2H-pyrido[1,2-a]pyrazin-6-yl]-2,3-dimethylphenoxy}propan-1-ol;
(2Z)-1-{4-[(6R,9aS)-2-(4-chloro-3-methoxyphenyl)octahydro-2H-pyrido[1,2-a]pyrazin-6-yl]-2,3-dimethylphenoxy}-3,3-dimethylbutan-2-one oxime;
2-{4-[(6R,9aS)-2-(4-chloro-3-methoxyphenyl)octahydro-2H-pyrido[1,2-a]pyrazin-6-yl]-2,3-dimethylphenoxy}ethanol;
2-{4-[(6R,9aS)-2-(4-fluoro-3-methoxyphenyl)octahydro-2H-pyrido[1,2-a]pyrazin-6-yl]-2,3-dimethylphenoxy}ethanol;
(2R)-1-amino-3-{4-[(6R,9aS)-2-(4-chloro-3-methoxyphenyl)octahydro-2H-pyrido[1,2-a]pyrazin-6-yl]-2,3-dimethylphenoxy}propan-2-ol;
(2S)-1-amino-3-{4-[(6R,9aS)-2-(4-chloro-3-methoxyphenyl)octahydro-2H-pyrido[1,2-a]pyrazin-6-yl]-2,3-dimethylphenoxy}propan-2-ol;
N-((2R)-3-{4-[(6R,9aS)-2-(4-chloro-3-methoxyphenyl)octahydro-2H-pyrido[1,2-a]pyrazin-6-yl]-2,3-dimethylphenoxy}-2-hydroxypropyl)acetamide;
N-((2S)-3-{4-[(6R,9aS)-2-(4-chloro-3-methoxyphenyl)octahydro-2H-pyrido[1,2-a]pyrazin-6-yl]-2,3-dimethylphenoxy}-2-hydroxypropyl)acetamide;
(1R)-2-(acetylamino)-1-({4-[(6R,9aS)-2-(4-chloro-3-methoxyphenyl)octahydro-2H-pyrido[1,2-a]pyrazin-6-yl]-2,3-dimethylphenoxy}methyl)ethyl acetate;
(1S)-2-(acetylamino)-1-({4-[(6R,9aS)-2-(4-chloro-3-methoxyphenyl)octahydro-2H-pyrido[1,2-a]pyrazin-6-yl]-2,3-dimethylphenoxy}methyl)ethyl acetate;
4-{4-[(6R,9aS)-2-(4-fluoro-3-methoxyphenyl)octahydro-2H-pyrido[1,2-a]pyrazin-6-yl]-2,3-dimethylphenoxy}butanoic acid;
4-{4-[(6R,9aS)-2-(4-fluoro-3-methoxyphenyl)octahydro-2H-pyrido[1,2-a]pyrazin-6-yl]-2,3-dimethylphenoxy}butanamide;
4-{4-[(6R,9aS)-2-(4-fluoro-3-methoxyphenyl)octahydro-2H-pyrido[1,2-a]pyrazin-6-yl]-2,3-dimethylphenoxy}-N-methylbutanamide;
4-{4-[(6R,9aS)-2-(4-fluoro-3-methoxyphenyl)octahydro-2H-pyrido[1,2-a]pyrazin-6-yl]-2,3-dimethylphenoxy}-N,N-dimethylbutanamide;
1-{4-[(6R,9aS)-2-(4-fluoro-3-methoxyphenyl)octahydro-2H-pyrido[1,2-a]pyrazin-6-yl]-2,3-dimethylphenoxy}acetone;
1-{4-[(6R,9aS)-2-(4-fluoro-3-methoxyphenyl)octahydro-2H-pyrido[1,2-a]pyrazin-6-yl]-2,3-dimethylphenoxy}propan-2-ol;
(2E)-1-{4-[(6R,9aS)-2-(4-fluoro-3-methoxyphenyl)octahydro-2H-pyrido[1,2-a]pyrazin-6-yl]-2,3-dimethylphenoxy}acetone oxime;
N-(3-{4-[(6R,9aS)-2-(4-fluoro-3-methoxyphenyl)octahydro-2H-pyrido[1,2-a]pyrazin-6-yl]-2,3-dimethylphenoxy}propyl)acetamide;
(2E)-1-{4-[(6R,9aS)-2-(4-chloro-3-methoxyphenyl)octahydro-2H-pyrido[1,2-a]pyrazin-6-yl]-2,3-dimethylphenoxy}acetone oxime;
(2E)-1-{4-[(6R,9aS)-2-(4-chloro-3-methoxyphenyl)octahydro-2H-pyrido[1,2-a]pyrazin-6-yl]-2,3-dimethylphenoxy}acetone O-methyloxime;
N-(3-{4-[(6R,9aS)-2-(4-chloro-3-methoxyphenyl)octahydro-2H-pyrido[1,2-a]pyrazin-6-yl]-2,3-dimethylphenoxy}propyl)-N-methylacetamide;
2-(4-{[4-(4-chloro-3-methoxyphenyl)piperazin-1-yl]methyl}-2-methoxyphenoxy)-N,N-dimethylethanamine;
2-(4-{1-[4-(4-chloro-3-methoxyphenyl)piperazin-1-yl]ethyl}-2-methoxyphenoxy)-N,N-dimethylethanamine;
2-(2-chloro-5-{4-[1-(3,4-dimethoxyphenyl)ethyl]piperazin-1-yl}phenoxy)-N,N-dimethylethanamine;
3-(2-chloro-5-{4-[1-(3,4-dimethoxyphenyl)ethyl]piperazin-1-yl}phenoxy)-N,N-dimethylpropan-1-amine;
ethyl 4-(4-{[4-(4-chloro-3-methoxyphenyl)piperazin-1-yl]methyl}-2-methoxyphenoxy)butanoate;
6-{[4-(4-chloro-3-methoxyphenyl)piperazin-1-yl]methyl}-2H-chromen-2-one;
1-(4-chloro-3-methoxyphenyl)-4-{1-[4-(2-methoxyethoxy)-2,3-dimethylphenyl]ethyl}piperazine;
1-(4-chloro-3-methoxyphenyl)-4-{(1R)-1-[4-(2-methoxyethoxy)-2,3-dimethylphenyl]ethyl}piperazine;
1-(4-chloro-3-methoxyphenyl)-4-{(1S)-1-[4-(2-methoxyethoxy)-2,3-dimethylphenyl]ethyl}piperazine;
3-(4-{1-[4-(4-chloro-3-methoxyphenyl)piperazin-1-yl]ethyl}-2,3-dimethylphenoxy)-N-methylpropan-1-amine;
3-(4-{1-[4-(4-chloro-3-methoxyphenyl)piperazin-1-yl]ethyl}-2,3-dimethylphenoxy)-N-ethylpropan-1-amine;
3-(4-{1-[4-(4-chloro-3-methoxyphenyl)piperazin-1-yl]ethyl}-2,3-dimethylphenoxy)-N-(cyclopropylmethyl)propan-1-amine;
3-(4-{1-[4-(4-chloro-3-methoxyphenyl)piperazin-1-yl]ethyl}-2,3-dimethylphenoxy)-N-(2-methoxyethyl)propan-1-amine;
3-(4-{1-[4-(4-chloro-3-methoxyphenyl)piperazin-1-yl]ethyl}-2,3-dimethylphenoxy)-N-isopropylpropan-1-amine;
N-[3-(4-{1-[4-(4-chloro-3-methoxyphenyl)piperazin-1-yl]ethyl}-2,3-dimethylphenoxy)propyl]cyclopentanamine;
3-(4-{1-[4-(4-chloro-3-methoxyphenyl)piperazin-1-yl]ethyl}-2,3-dimethylphenoxy)-N,N-dimethylpropan-1-amine;
1-(4-chloro-3-methoxyphenyl)-4-{1-[4-(cyclopropylmethoxy)-2,3-dimethylphenyl]ethyl)}piperazine;
1-(4-chloro-3-methoxyphenyl)-4-{1-[4-(3-ethoxypropoxy)-2,3-dimethylphenyl]ethyl}piperazine;
3-(4-{1-[4-(4-chloro-3-methoxyphenyl)piperazin-1-yl]ethyl}-2-methoxyphenoxy)-N-isopropylpropan-1-amine;
1-[1-(4-butoxy-3-methoxyphenyl)ethyl]-4-(4-chloro-3-methoxyphenyl)piperazine;
1-(4-chloro-3-methoxyphenyl)-4-{1-[4-(cyclopropylmethoxy)-3-methoxyphenyl]ethyl}piperazine;
1-[1-(4-butoxy-2,3-dimethylphenyl)ethyl]-4-(4-chloro-3-methoxyphenyl)piperazine;
1-(4-chloro-3-methoxyphenyl)-4-{1-[3-methoxy-4-(2-methoxyethoxy)phenyl]ethyl}piperazine;
2-(4-{1-[4-(4-chloro-3-methoxyphenyl)piperazin-1-yl]ethyl}-2,3-dimethylphenoxy)acetamide;
(4-{1-[4-(4-chloro-3-methoxyphenyl)piperazin-1-yl]ethyl}-2,3-dimethylphenoxy)acetonitrile;
4-(4-{1-[4-(4-chloro-3-methoxyphenyl)piperazin-1-yl]ethyl}-2,3-dimethylphenoxy)butanenitrile;
1-(4-bromo-3-methoxyphenyl)-4-{1-[4-(2-methoxyethoxy)-2,3-dimethylphenyl]ethyl}piperazine;
1-{1-[4-(2-methoxyethoxy)-2,3-dimethylphenyl]ethyl}4-(3-methoxy-4-methylphenyl)piperazine;
1-(2,3-dihydro-1-benzofuran-6-yl)-4-{1-[4-(2-methoxyethoxy)-2,3-dimethylphenyl]ethyl}piperazine;
1-{1-[4-(2-methoxyethoxy)-2,3-dimethylphenyl]ethyl}-4-(3-methoxyphenyl)piperazine;
1-{1-[4-(2-methoxyethoxy)-2,3-dimethylphenyl]ethyl}-4-(3-methoxy-4-vinylphenyl)piperazine;
1-(3,4-difluoro-5-methoxyphenyl)-4-{1-[4-(2-methoxyethoxy)-2,3-dimethylphenyl]ethyl}piperazine;
3-(4-{1-[4-(4-chloro-3-methoxyphenyl)piperazin-1-yl]ethyl}-2,3-dimethylphenoxy)-N-methoxypropan-1-amine;
2-(4-{[3-(4-Chloro-3-methoxyphenyl)-3-hydroxy-8-azabicyclo[3.2.1]oct-8-yl]methyl}-2,3-dimethylphenoxy)-N,N-dimethylacetamide;
2-(4-{[3-(4-Chloro-3-methoxyphenyl)-3-hydroxy-8-azabicyclo[3.2.1]oct-8-yl]methyl}-2,3-dimethylphenoxy)-N-methylacetamide;
3-(4-Chloro-3-methoxyphenyl)-8-{4-[3-(dimethylamino)propoxy]-2,3-dimethylbenzyl}-8-azabicyclo[3.2.1]octan-3-ol;
3-(4-Chloro-3-methoxyphenyl)-8-[4-(3-hydroxypropoxy)-2,3-dimethylbenzyl]-8-azabicyclo[3.2.1]octan-3-ol;
3-(4-Chloro-3-methoxyphenyl)-8-[4-(2-methoxyethoxy)-2,3-dimethylbenzyl]-8-azabicyclo[3.2.1]octan-3-ol; or
3-(4-Chloro-3-methoxyphenyl)-8-[4-(2-hydroxyethoxy)-2,3-dimethylbenzyl]-8-azabicyclo[3.2.1]octan-3-ol.
